SEQUENCE LISTING (3 sequences)

SEQ ID NO: 1 - protein, 6 aa, Artificial Sequence
  2-Furoyl-LIGRLO-NH2 peptide
  VARIANT 1: Leu = N-(2-Furoyl)-Leu
  MOD_RES 6: AMIDATION
  VARIANT 6: Xaa = Ornithine
LIGRLX

SEQ ID NO: 2 - protein, 7 aa, Artificial Sequence
  synthetic peptide comprising tethered ligand
RSLIGKV

SEQ ID NO: 3 - protein, 6 aa, Artificial Sequence
  tethered ligand
SLIGKV

(19) United States
(12) Patent Application Publication
(10) Pub. No.: US 2024/0066027 A1
(43) Pub. Date: Feb. 29, 2024

(54) NOVEL PAR-2 INHIBITORS

(71) Applicant: DOMAIN THERAPEUTICS, Illkirch-Graffenstaden (FR)

(72) Inventors: Stanislas MAYER, Illkirch-Graffenstaden (FR); Anne-Laure BLAYO, Illkirch-Graffenstaden (FR); Mickaël FER, Illkirch-Graffenstaden (FR); Anaïs BARRÉ, Illkirch-Graffenstaden (FR); Mathieu MICHAUT, Illkirch-Graffenstaden (FR); Gaël HOMMET, Illkirch-Graffenstaden (FR); Stephan SCHANN, Illkirch-Graffenstaden (FR); Baptiste RUGERI, Illkirch-Graffenstaden (FR)

(21) Appl. No.: 18/254,261

(22) PCT Filed: Dec. 3, 2021
(86) PCT No.: PCT/EP2021/084285

§ 371 Date: May 24, 2023

(30) Foreign Application Priority Data
Dec. 3, 2020 (EP) 20306495.1

(51) Int. Cl. A61K 31/496; C07D 471/04; C07D 519/00; A61K 31/5377; A61K 31/46; A61K 31/438; A61K 31/437; A61K 31/551; A61K 31/4545; A61K 31/553; A61K 31/5386; A61K 39/395

(52) U.S. Cl. CPC A61K 31/496; C07D 471/04; C07D 519/00; A61K 31/5377; A61K 31/46; A61K 31/438; A61K 31/437; A61K 31/551; A61K 31/4545; A61K 31/553; A61K 31/5386; A61K 39/3955

(57) ABSTRACT

The present invention provides novel compounds of formula (I) and pharmaceutical compositions containing these compounds. The compounds of formula (I) can act as PAR-2 inhibitors, which renders these compounds highly advantageous for use in therapy, particularly in the treatment or prevention of pain, an autoimmune disorder, an autoinflammatory disorder, an inflammatory disorder, a central nervous system disorder, spinal cord injury, a metabolic disorder, a gastrointestinal disorder, a cardiovascular disorder, a fibrotic disorder, a respiratory disorder, a skin disorder, an allergic disorder, or cancer.

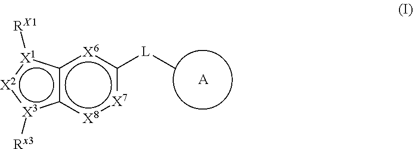

The present invention provides novel compounds of formula (I) and pharmaceutical compositions containing these compounds. The compounds of formula (I) can act as PAR-2 inhibitors, which renders these compounds highly advantageous for use in therapy, particularly in the treatment or prevention of pain, an autoimmune disorder, an autoinflammatory disorder, an inflammatory disorder, a central nervous system disorder, spinal cord injury, a metabolic disorder, a gastrointestinal disorder, a cardiovascular disorder, a fibrotic disorder, a respiratory disorder, a skin disorder, an allergic disorder, or cancer.

The Protease-Activated Receptors (PARs) Family

G Protein-Coupled Receptors (GPCRs) form the largest family of human membrane proteins (˜800 members) and are involved in many physiological processes. Compounds targeting GPCRs also represent approximately 27% of the global market for therapeutic drugs (Hauser et al., Nat. Rev. Drug Discov., 2017, 16(12):829-842). 2% of the human genome code for proteases (also called proteinases) which suggests their importance in the correct functioning of the body (Hollenberg et al., Br. J. Pharmacol., 2014, 171(5):1180-94). Indeed, it has been shown that certain soluble and membrane-bound proteinases can regulate cell function by cleaving GPCRs at the cell surface to activate or inactivate receptors such as the Protease-Activated Receptors (PARs). The PARs family is composed of four members (PAR-1, PAR-2, PAR-3 and PAR-4) and belongs to the class A GPCR-receptor sub-family (Marcfarlane et al., Pharmacological Reviews, 2001, 475(7357):519-23). They are expressed in widely diverse cells such as platelets, immune cells, endothelial cells, myocytes, astrocytes, neurons, epithelial cells and fibroblasts and involved in a large set of physiological and pathophysiological functions (Ossovskaya et al., Physiol. Rev., 2004, 84(2):579-621).

PAR-2: Mechanism of Action

Activation of PARs involves the cleavage of the extracellular N-terminal part of the receptor by proteases at a specific site. This unmasks an amino-acid sequence in the amino terminus that folds back to act as a “tethered ligand” (TL): it binds to a conserved region in the second extracellular loop of the cleaved receptor and triggers intra-cellular signalling (Ossovskaya et al., Physiol. Rev., 2004, 84(2):579-621; Hollenberg et al., Br. J. Pharmacol., 2014, 171(5):1180-94).

PAR-2 is activated by several host and pathogen-derived serine proteases such as trypsin, mast cell tryptase, kallikreins and members of the coagulation cascade TF-FVIIa and FVa-FXa. These proteases cleave at R³¹↓S³⁵LIGKV and unmask the tethered ligand SLIGKV in humans. Artificially, in vitro, synthetic peptides corresponding to the TL (SLIGKV) can activate the receptor without cleavage.

Activation of PAR-2 induces several signalling cascades involving a number of G proteins such as G_{q}, Gᵢ, and G_{12/13}. The pathway best described so far involves its interaction with G_{q} and the mobilization of intracellular calcium that influences the function of several cell types. After repeated activations, PAR2 is rapidly desensitized via its endocytosis by a β-arrestin-dependent mechanism and its targeting to the lysosomes (Ossovskaya et al., Physiol. Rev., 2004, 84(2):579-621).

PAR-2 in Physiological Conditions

PAR-2 has been shown to have a key function in multiple organs (Ossovskaya et al., Physiol. Rev., 2004, 84(2):579-621). PAR-2 is expressed in the brain within neurons and glial cells. It is also found in the periphery in spinal afferent neurons and nociceptive DRG neurons. PAR-2 signalling has been involved in the survival, sensitization of these cells and their signal transmission, thereby controlling neuronal damage, inflammation and pain.

PAR-2 is involved in the function of the cardiovascular system. Indeed, its activation can induce the relaxation or contraction of some vessels such as pulmonary arteries, coronary and intramyocardial arteries, therefore regulating the blood flow. It also controls inflammation and repair of the endothelium which influences vascular permeability.

PAR-2 expression has been detected within the gastrointestinal system in the small intestine, colon, liver, pancreas and stomach. Its activation has been involved in the regulation of ion transport from the intestinal mucosa, contraction of gastric longitudinal muscle, pancreatic, salivary and gastric secretions, excitation of myenteric neurons, intestinal barrier integrity, release of prostaglandins from enterocytes. PAR-2 therefore plays a key role in controlling fluid secretion, intestinal inflammation, and gastro-intestinal hyperalgesia.

PAR-2 is involved in airways function since it is expressed by epithelial and endothelial cells in the lungs. Its activation has been shown to regulate bronchodilation or bronchoconstriction (depending on the experimental system used), ion transport in the airway epithelium, proliferation and activation of airway smooth muscle cells and lung fibroblasts.

PAR-2 can thus regulate airway resistance, lung inflammation and lung fibrosis.

In the skin, PAR-2 expression has been detected in keratinocytes, microvasculature and immune cells. Its activation has been involved in skin pigmentation, skin inflammation, and wound healing.

Finally, PAR-2 expression has been detected in immune cells such as macrophages where it influences cell maturation and cytokine secretion, thereby regulating inflammation.

PAR-2 in Pathological Conditions

Since PAR-2 regulates numerous and diverse biological processes, it is not surprising that its dysfunction is involved in as many pathological conditions.

PAR-2 is expressed in the brain, dorsal root ganglia, spinal afferent neurons and nociceptive DRG neurons. Its activation by proteases such as the tryptase released by mast cells leads to calcium and cAMP signalling (Steinhoff et al., Nat Med, 2000, 6(2):151-8; Zhao et al., J Biol Chem., 2015, 290(22):13875-87). This promotes inflammation and hyperalgesia through the release of CGRP (calcitonine gene-related peptide) and SP (substance P) from spinal afferent neurons and the sensitization of Transient Receptors Potential Vanilloid (TRPV) TRPV1 and TRPV4 in sensory neurons (Vergnolle et al., Nat Med, 2001, 6(2):151-8; Steinhoff et al., Nat Med, 2000, 6(2):151-8; Amadesi et al., J Neurosci, 2004, 24(18):4300-12; Grant et al., J Physiol, 2007, 578 (Pt 3), 715-33; Jimenez Vargas et al., Proc Natl Acad Sci USA, 2018, 115(31):E7438-E7447). This is supported by the large amount ofin vivo data available in the literature demonstrating that inhibition of PAR-2 reduces inflammatory pain, neuropathic pain, cancer pain and treatment-induced pain in animal models (Bao et al., Expert Opin Ther Targets, 2014; 18(1):15-27; Chen et al., Neuroscience, 2011, 193, 440-51). PAR-2 is therefore clearly involved in the generation and the transmission of the pain signal, neurogenic inflammation and nociception.

The expression of PAR-2 and proteases is elevated in the spinal cord after a contusion-compression injury (Radulovic et al., Neurobiol Dis, 2015, 83, 75-89; Li et al, Physiol. Res., 2019, 68(2):305-316). Its activation can result in cAMP signalling in oligodendrocytes (Yoon et al., Glia, 2017, 65(12):2070-2086). Experiments in vitro and in vivo in rodents have shown that the inhibition of PAR-2 signalling during experimental spinal cord injury reduces inflammation, scar formation and mechanical and thermal hyperalgesia and improves remyelination of oligodendrocytes and locomotor recovery (Radulovic et al., Neurobiol Dis, 2015, 83, 75-89; Li et al, Physiol. Res., 2019, 68(2):305-316; Yoon et al., Glia, 2017, 65(12):2070-2086; Li et al, Physiol. Res., 2019, 68(2):305-316; Wei et al, Physiol. Res., 2016, 65(1):145-53). PAR-2 inhibitors can thus improve recovery from spinal cord injuries.

Disorders of the immune system are at the basis of numerous diseases. In all cases, the immune system attacks the normal constituents of the organism considering them as foreign. It becomes pathogenic and induces lesions on a specific organ (e.g., type 1 diabetes in the pancreas or multiple sclerosis in the brain) or systemically (e.g., rheumatoid arthritis or systemic lupus erythematosus, SLE).

Cytokines are small proteins involved in cell signalling that orchestrate the immune response. Their dysregulation is at the basis of the pathogenesis of autoinflammatory diseases. These conditions are characterized by immune activation, infiltration and abnormal cytokine production. They include conditions such as: rheumatologic inflammatory diseases, skin inflammatory diseases, lung inflammatory diseases, muscle inflammatory diseases, bowel inflammatory diseases, brain inflammatory diseases and autoimmune diseases.

While autoinflammatory diseases evolve chronically, some conditions can lead to an acute immune disorder. Indeed, a sudden excessive and uncontrolled release of pro-inflammatory cytokines, also called cytokine storm, has been observed in graft-versus-host disease, multiple sclerosis, pancreatitis, multiple organ dysfunction syndrome, viral diseases, bacterial infections, hemophagocytic lymphohistiocytosis, and sepsis (Gerlach H, F1000Res, 2016, 5, 2909; Tisoncik J R et al., Microbiol Mol Biol Rev, 2012, 76(1):16-32). In these conditions, a dysregulated immune response and subsequent hyperinflammation may lead to multiple organ failure that can be fatal.

Because PAR-2 influences the production of inflammatory cytokines and the function of diverse organs, numerous studies have demonstrated that it is a promising therapeutic target for various autoinflammatory diseases.

The expression of proteinases and PAR-2 is significantly increased in organs directly involved in autoinflammatory diseases such as the coronary arteries of atherosclerotic patients (Jones et al., Arterioscler Thromb Vasc Biol, 2018, 38(6):1271-1282), the skin of atopic dermatitis and psoriasis patients (Nattkemper et al., Journal of Investigative Dermatology, 2018, 138:1311-1317), the joints of rheumatoid arthritis and osteoarthritis patients (Tindell et al., Rheum Int, 2012, 32(10):3077-86), the colon of inflammatory bowel disease patients (Christerson et al., J Crohns Colitis, 2009, 3(1):15-24; Kim et al., Inflamm Bowel Dis., 2003, 9(4):224-9), the lungs of idiopathic pulmonary fibrosis patients (Bardou et al., Am J Respir Crit Care Med, 2016, 193(8):847-60), the liver of non-alcoholic steatohepatitis patients (Rana et al., Mol Metab, 2019, 29:99-113), the area of active demyelination in the brain of multiple sclerosis patients (Noorbakhsh et al., J Exp Med, 2006, 203(2):425-35).

There, PAR-2 activation leads to calcium signalling in several cells such as osteoblasts, fibroblasts, monocytes, keratinocytes (Abraham et al, Bone, 2000, 26(1):7-14; Lin et al., J. Cell. Mol. Med., 2015, 19(6):1346-56; Johansson et al., J leukoc Biol, 2005, 78(4):967-75; Joo et al., Bio Mol Ther, 2016, 24(5):529-535). This signalling is associated with cell maturation and/or migration, activation as well as the secretion of inflammatory cytokines such as IL-8, IL-6, TNFα and IL-1β in various cell types such as vascular smooth muscle cells, synovial cells, monocytes, keratinocytes, astrocytes, chondrocytes, adipocytes and fibroblasts (Demetz et al., Atherosclerosis, 2010, 212:466-471; Kelso et al., Arthritis Rheum, 2007, 56(3):765-71; Johansson et al., J Leukoc Biol, 2005, 78(4):967-75; Steven et al., Innate Immun, 2013, 19(6):663-72; Kim et al., Bio Mol Ther, 2012, 20(5):463-9; Radulovic et al., Neurobiol Dis, 2015, 83, 75-89; Lin et al., J. Cell. Mol. Med., 2015, 19(6):1346-56; Bagher et al., Cell Communi and Signal, 2018, 16(1), 59; Huang et al, Aging, 2019, 11(24):12532-12545; Bandeanlou et al., Nat. Med., 2011, 17:1490-1497). PAR-2 signalling also influences tissue remodelling through its role in the survival of key cells such as neurons and chondrocytes in central nervous system disorders and rheumatologic inflammatory diseases respectively (Afkhami-Goli et al., J Immunol, 2007, 179(8):5493-503; Huang et al., Aging, 2019, 11(24):12532-12545), as well as the secretion of growth factors (e.g. CTGF) and extracellular components (e.g. collagen) (Lin et al., Mol. Med., 2015, 21(1):576-83; Chung et al., J Biol Chem, 2013, 288(52):37319-31). It is important to note that other signalling pathways such as cyclic AMP in alveolar macrophages and Gi in hepatocytes seem important to regulate cytokine secretion and steatosis respectively (Rayees et al., Cell Rep, 2019, 27(3):793-805.e4; Rana et al., Mol Metab, 2019, 29, 99-113).

In vivo, it has clearly been shown that the inhibition of PAR-2 signaling, either pharmacologically or by genetic modification, significantly reduced the symptoms of atherosclerosis, idiopathic pulmonary fibrosis, atopic dermatitis, multiple sclerosis, arthritis, non-alcoholic steatohepatitis and inflammatory bowel disease in mouse models (Jones et al., Arterioscler Thromb Vasc Biol, 2018, 38(6):1271-1282; Borensztajn et al., Am J Pathol, 2010, 177(6):2753-64; Moniaga et al., Am J Pathol, 2013, 182: 841e851; Noorbakhsh et al., J Exp Med, 2006, 203(2):425-35, Ferrell et al., J Clin Invest, 2003, 111(1):35-41; Rana et al., Mol Metab, 2019, 29:99-113; Hyun et al., Gut, 2008, 57(9):1222-9). PAR-2 therefore plays a key role in the molecular and cellular mechanisms underlying the pathogenesis of autoinflammatory diseases.

PAR-2-dependent inflammation can also impair cellular metabolism and promote insulin resistance which then leads to the pathogenesis of diabetes, obesity and metabolic syndrome. Indeed, PAR-2 expression in adipocyte tissues has been correlated with the increasing BMI of volunteer people and the inhibition of PAR-2 signaling attenuates the symptoms of metabolic disorders in mice (Lim et al., FASEB Journal, 2013, 27(12):4757-4767; Badeanlou et al., Nat. Med., 2011, 17(11):1490-1497).

Many airborne allergens from house dust mite and cockroach allergens contain protease activity. This protease activity can activate PAR-2 expressed on human airway epithelial cells, endothelial cells as well as immune cells and induce calcium signalling. This ultimately leads to the release of inflammatory cytokines and angiogenic response at the basis of the pathogenesis of cockroach allergy and allergic asthma (Do et al., Allergy, 2016, 71(4):463-74; Asosingh et al., J Clin Invest, 2018, 128(7):3116-3128). In vivo, functional blockade of PAR-2 in the airways during allergen challenge improves allergen-induced inflammation and airway hyperresponsiveness in mice (Asaduzzaman et al., Clin Exp Allergy, 2015, 45(12):1844-55).

The expression of PAR-2 and proteases is also significantly increased in many cancer types such as cervical squamous cell carcinoma, endocervical adenocarcinoma, colon adenocarcinoma, esophageal carcinoma, glioblastoma multiforme, acute myeloid leukemia, lung adenocarcinoma, lung squamous cell carcinoma, ovarian serous cystadenocarcinoma, pancreatic adenocarcinoma, prostate adenocarcinoma, rectum adenocarcinoma, stomach adenocarcinoma, testicular germ cell tumors, uterine corpus endometrial carcinoma, uterine carcinosarcoma, hepatocellular carcinoma, and breast cancer, which can be associated to poor prognosis (Kaufmann et al., Carcinogenesis, 2009, 30(9):1487-96; Su et al., Oncogene, 2009, 28(34):3047-57; Arakaki et al., nt. J. Mol. Sci. 2018, 19, 1886). The activation of this receptor in cancer cells can lead to several signalling cascades such as calcium, β-arrestin and Gi signalling (Kaufmann et al., J Cancer Res Clin Oncol, 2011, 137(6):965-73; Wu et al, Mol Med Rep, 2014, 10(6):3021-6; Ge et al., J Biol Chem, 2004, 279(53):55419-24). This ultimately controls cancer cell migration, proliferation, survival, and expression of inflammatory cytokines (Jiang et al., J Pharmacol Exp Ther, 2018, 364(2):246-257; Darmoul et al., British J Cancer, 2001, 85(5):772-9; Quan et al., Oncol Res., 2019, 27(7):779-788). The expression of PAR-2 on other cells of the tumor microenvironment, such as immune cells, fibroblasts, endothelial cells and DRG neurons, can also control the immune response to cancer cells, fibrosis, as well as angiogenesis and cancer-induced pain (Mubbach et al., Mol cancer, 2016, 15(1):54; Uusitalo-Jarvinen et al., Arteriocler Thromb Vasc Biol, 2007, 27(6):1456-62; D'Andrea et al, Am J Pathol, 2001, 158(6):2031-41; Graf et al, Sci Immunol, 2019, 4(39):eaaw8405; Qian at al., OncolLett, 2018, 16(2):1513-20; Tu et al, J Neurosci, 2021, 41(1):193-210). In vivo, the inhibition of PAR-2 has been shown to be an efficient way of reducing tumor growth and increasing survival in mouse models of different cancers such as breast cancer, liver cancer and colon cancer (Versteeg et al., Cancer Res, 2008, 68(17):7219-27; Sun et al., World J Gastroenterol, 2018, 24(10):1120-1133; Quan et al., Oncol Res., 2019, 27(7):779-788). Importantly, inhibition of PAR2 or one of its ligands led to reduced infiltration of immune-supressive Tumor Associated Macrophages and regulatory T cells while increasing cytotoxic T cells in the tumor as well as increasing antigen presenting cells in the draining lymph nodes in several syngeneic mouse models; this unleashed the anti-tumoral immune response and increased the potency of immune-checkpoint inhibitors currently used in the clinic (Graf et al, Sci Immunol, 2019, 4(39):eaaw8405). PAR-2 therefore constitutes a promising therapeutic target in oncology and immune-oncology.

Considering the role of PAR-2 in several pathophysiological conditions, inhibitors of this receptor can have therapeutic applications in a wide variety of human diseases. This has drawn a great interest from pharmaceutical industry to develop such compounds. Various PAR-2 inhibitors have been proposed, for example, in: Yau et al., Expert Opin Ther Pat, 2016, 26(4):471-83; Jiang et al., J Pharmacol Exp Ther, 2018, 364(2):246-57; WO 2004/002418; WO 2005/030773; WO 2012/012843; WO 2012/026765; WO 2012/026766; WO 2012/101453; WO 2015/048245; WO 2016/154075; WO 2017/194716; WO 2017/197463; WO 2018/043461; WO 2018/057588; and WO 2019/199800. However, despite the efforts made in the past 10 years, no PAR-2 inhibitor has reached the market yet (Yau et al., Expert Opin Ther Pat., 2016, 26(4):471-83). There is therefore still an unmet need for novel and/or improved PAR-2 inhibitors with high potency, selectivity and bioavailability.

The present invention addresses this need and solves the problem of providing novel and highly potent PAR-2 inhibitors. In particular, it has surprisingly been found that the compounds of formula (I) as provided herein are potent inhibitors of PAR-2 signalling, which renders these compounds advantageous for use in therapy, including in particular in the treatment or prevention of pain, an autoimmune disorder, an autoinflammatory disorder, an inflammatory disorder, a central nervous system disorder, spinal cord injury, a metabolic disorder, a gastrointestinal disorder, a cardiovascular disorder, a fibrotic disorder, a respiratory disorder, a skin disorder, an allergic disorder, or cancer.

Accordingly, the present invention provides a compound of the following formula (I)

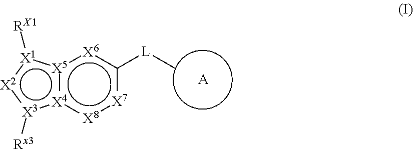

or a pharmaceutically acceptable salt or solvate thereof.

As depicted in formula (I), the bicyclic ring system containing the ring atoms X¹ to X⁸ is aromatic.

One, two, three or four of the ring atoms X¹ to X⁸ are nitrogen atoms, and all remaining ring atoms are carbon atoms.

Any among the ring atoms X², X⁶, X⁷ and X⁸ that is a carbon atom is optionally substituted with a group R^{X}.

Each R^{X} is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-NH—OH, —(C₀₋₃ alkylene)-N(C₁₋₅ alkyl)-OH, —(C₀₋₃ alkylene)-NH—O(C₁₋₅ alkyl), —(C₀₋₃ alkylene)-N(C₁₋₅ alkyl)-O(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-NH—COO(C₁₋₅ alkyl), —(C₀₋₃ alkylene)-N(C₁₋₅ alkyl)-COO(C₁₋₅ alkyl), —(C₀₋₃ alkylene)-O—CO—NH(C₁₋₅ alkyl), —(C₀₋₃ alkylene)-O—CO—N(C₁₋₅ alkyl)(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-SO—(C₁₋₅ alkyl), —(C₀₋₃ alkylene)-SO₂—(C₁₋₅ alkyl), —(C₀₋₃ alkylene)-carbocyclyl, —(C₀₋₃ alkylene)-heterocyclyl, and -L¹-R^{L1}, wherein the carbocyclyl group in said —(C₀₋₃ alkylene)-carbocyclyl and the heterocyclyl group in said —(C₀₋₃ alkylene)-heterocyclyl are each optionally substituted with one or more groups R^{Cyc}.

R^{X1} is selected from C₁₋₅ alkyl, C₂₋₅ alkenyl, C₂₋₅ alkynyl, —(C₀₋₅ alkylene)-carbocyclyl, and —(C₀₋₅ alkylene)-heterocyclyl, wherein said alkyl, said alkenyl, said alkynyl, the alkylene group in said —(C₀₋₅ alkylene)-carbocyclyl, and the alkylene group in said —(C₀₋₅ alkylene)-heterocyclyl are each optionally substituted with one or more groups R^{X11}, wherein one or more —CH₂— units comprised in said alkyl, said alkenyl, said alkynyl, in the alkylene group in said —(C₀₋₅ alkylene)-carbocyclyl, or in the alkylene group in said —(C₀₋₅ alkylene)-heterocyclyl are each optionally replaced by a group independently selected from —O—, —NH—, —N(C₁₋₅ alkyl)-, —CO—, —S—, —SO—, and —SO₂—, and further wherein the carbocyclyl group in said —(C₀₋₅ alkylene)-carbocyclyl and the heterocyclyl group in said —(C₀₋₅ alkylene)-heterocyclyl are each optionally substituted with one or more groups R^{Cyc}.

Each R^{X11} is independently selected from —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O—(C₁₋₅ haloalkyl), —CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO—(C₁₋₅ alkyl), —SO₂—(C_{1- 5} alkyl), carbocyclyl, heterocyclyl, and -L¹-R^{L1}, wherein said carbocyclyl and said heterocyclyl are each optionally substituted with one or more groups R^{Cyc}.

R^{X3} is -L^{X3}-carbocyclyl or -L^{X3}-heterocyclyl, wherein the carbocyclyl in said -L^{X3}-carbocyclyl or the heterocyclyl in said -L^{X3}-heterocyclyl is optionally substituted with one or more groups R^{X31}.

L^{X3} is independently selected from a bond, —C(R^{LX3})(R^{LX3})—, —O—, —S—, —SO—, —SO₂—, —CO—, and —N(R^{LX3})—, wherein each R^{LX3} is independently hydrogen or C₁₋₅ alkyl, and further wherein two groups R^{LX3} which are attached to the same carbon atom may also be mutually joined to form, together with the carbon atom that they are attached to, a cycloalkyl or a heterocycloalkyl.

Each R^{X31} is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-NH—OH, —(C₀₋₃ alkylene)-N(C₁₋₅ alkyl)-OH, —(C₀₋₃ alkylene)-NH—O(C₁₋₅ alkyl), —(C₀₋₃ alkylene)-N(C₁₋₅ alkyl)-O(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-NH—COO(C₁₋₅ alkyl), —(C₀₋₃ alkylene)-N(C₁₋₅ alkyl)-COO(C₁₋₅ alkyl), —(C₀₋₃ alkylene)-O—CO—NH(C₁₋₅ alkyl), —(C₀₋₃ alkylene)-O—CO—N(C₁₋₅ alkyl)(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-SO—(C₁₋₅ alkyl), —(C₀₋₃ alkylene)-SO₂—(C₁₋₅ alkyl), —(C₀₋₃ alkylene)-carbocyclyl, —(C₀₋₃ alkylene)-heterocyclyl, and -L¹-R^{L1}, wherein the carbocyclyl group in said —(C₀₋₃ alkylene)-carbocyclyl and the heterocyclyl group in said —(C₀₋₃ alkylene)-heterocyclyl are each optionally substituted with one or more groups R^{Cyc}.

L is selected from —CO—, —SO— and —SO₂—.

Ring A is a 5 to 14 membered heterocyclyl which is attached via a ring nitrogen atom to group L, wherein said heterocyclyl is optionally substituted with one or more groups R^{A}.

Each R^{A} is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-NH—OH, —(C₀₋₃ alkylene)-N(C₁₋₅ alkyl)-OH, —(C₀₋₃ alkylene)-NH—O(C₁₋₅ alkyl), —(C₀₋₃ alkylene)-N(C₁₋₅ alkyl)-O(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-NH—COO(C₁₋₅ alkyl), —(C₀₋₃ alkylene)-N(C₁₋₅ alkyl)-COO(C₁₋₅ alkyl), —(C₀₋₃ alkylene)-O—CO—NH(C₁₋₅ alkyl), —(C₀₋₃ alkylene)-O—CO—N(C₁₋₅ alkyl)(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-SO—(C₁₋₅ alkyl), —(C₀₋₃ alkylene)-SO₂—(C₁₋₅ alkyl), —(C₀₋₃ alkylene)-carbocyclyl, —(C₀₋₃ alkylene)-heterocyclyl, and -L¹-R^{L1}, wherein the carbocyclyl group in said —(C₀₋₃ alkylene)-carbocyclyl and the heterocyclyl group in said —(C₀₋₃ alkylene)-heterocyclyl are each optionally substituted with one or more groups R^{Cyc}; and further wherein any two groups R^{A}, which are attached to the same carbon ring atom of ring A, may also be mutually joined to form, together with the carbon ring atom that they are attached to, a cycloalkyl or a heterocycloalkyl, wherein said cycloalkyl or said heterocycloalkyl is optionally substituted with one or more groups R^{Cyc}.

Each R^{Cyc} is independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O—(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), —P(═O)(—OH)(—OH), —P(═O)(—OH)(—O—C₁₋₅ alkyl), —P(═O)(—O—C₁₋₅ alkyl)(-O—C₁₋₅ alkyl), —(C₀₋₃ alkylene)-cycloalkyl, —(C₀₋₃ alkylene)-heterocycloalkyl, and -L¹-R^{L1}.

Each L¹ is independently selected from a covalent bond, C₁₋₇ alkylene, C₂₋₇ alkenylene, and C₂₋₇ alkynylene, wherein said alkylene, said alkenylene and said alkynylene are each optionally substituted with one or more groups independently selected from halogen, C₁₋₅ haloalkyl, —O—(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl), and further wherein one or more —CH₂— units comprised in said alkylene, said alkenylene or said alkynylene are each optionally replaced by a group independently selected from —O—, —NH—, —N(C₁₋₅ alkyl)-, —CO—, —S—, —SO—, and —SO₂—.

Each R^{L1} is independently selected from —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O—(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C_{1- 5} alkyl), aryl, heteroaryl, cycloalkyl, and heterocycloalkyl, wherein said aryl, said heteroaryl, said cycloalkyl, and said heterocycloalkyl are each optionally substituted with one or more groups independently selected from C₁₋₅ alkyl, C₂₋₅ alkenyl, C₂₋₅ alkynyl, halogen, C₁₋₅ haloalkyl, —O—(C₁₋₅ haloalkyl), —CN, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO—(C₁₋₅ alkyl), and —SO₂—(C₁₋₅ alkyl).

In accordance with the present invention, the following conditions apply to the compounds of formula (I):
if X¹, X² and X⁷ are nitrogen atoms, X³, X⁴, X⁵, X⁶ and X⁸ are carbon atoms, and L is —CO—, then R^{X3} is aryl, -L^{X3}-cycloalkyl, or monocyclic heteroaryl, wherein said aryl, the cycloalkyl in said -L^{X3}-cycloalkyl, or said heteroaryl is optionally substituted with one or more groups R^{X31};
if X², X³, X⁶ and X⁸ are nitrogen atoms, X¹, X⁴, X⁵ and X⁷ are carbon atoms, L is —CO—, and ring A is a monocyclic heterocyclyl which is attached via a ring nitrogen atom to group L and which is optionally substituted with one or more groups R^{A}, then the carbon atom in position X⁷ is not substituted with C₁₋₅ alkyl, —OH, —O(C₁₋₅ alkyl), —NH₂, —NH(C₁₋₅ alkyl), —N(C₁₋₅ alkyl)(C₁₋₅ alkyl), or phenyl;
if X¹ and X² are nitrogen atoms, X³, X⁴ and X⁵ are carbon atoms, L is —CO—, and R^{X1} is pyrimidin-2-yl which is optionally substituted with one or more groups R^{Cyc}, then R^{X3} is not cycloalkyl;
if X³ is a nitrogen atom, X¹, X⁴ and X⁵ are carbon atoms, and R^{X1} is phenyl, pyridin-2-yl or pyrimidin-2-yl wherein said phenyl, said pyridin-2-yl or said pyrimidin-2-yl is optionally substituted with one or more groups R^{Cyc}, then R^{X3} is not cyclopropyl;
if X³ and X⁸ are nitrogen atoms, X¹, X², X⁴, X⁵, X⁶ and X⁷ are carbon atoms, L is —CO—, and ring A is 2,5-dimethyl-4-(4-fluorobenzyl)-piperazin-1-yl, then R^{X3} is not 4-methoxybenzyl;
if X¹ is a nitrogen atom, X², X³, X⁴, X⁵, X⁶, X⁷ and X⁸ are carbon atoms, and L is —CO—, then ring A is not 4-(C₁₋₅ alkyl)-piperazin-1-yl or 4-(C₃₋₇ cycloalkyl)-piperazin-1-yl;
if X¹ is a nitrogen atom, X³, X⁴, X⁵, X⁶, X⁷ and X⁸ are carbon atoms, and L is —CO—, then R^{X3} is not a 9-membered bicyclic heteroaryl which is optionally substituted with one or more groups R^{X31};
if X¹ is a nitrogen atom, X², X³, X⁴, X⁵, X⁶, X⁷ and X⁸ are carbon atoms, L is —CO—, and R^{X1} is —CH₃ or —SO₂—CH₃, then R^{X3} is not —CH₂-heterocycloalkyl wherein the heterocycloalkyl in said —CH₂-heterocycloalkyl is optionally substituted with one or more groups R^{X31};
if X¹ is a nitrogen atom, X², X³, X⁴, X⁵, X⁶, X⁷ and X⁸ are carbon atoms, L is —CO—, ring A is morpholin-4-yl, and R^{X1} is 4-carboxythiazol-2-yl, then R^{X3} is not 4-sulfamoylbenzyl;
if X³ is a nitrogen atom, X¹, X², X⁴, X⁵, X⁶, X⁷ and X⁸ are carbon atoms, L is —CO—, ring A is morpholin-4-yl, and R^{X1} is 4-sulfamoylbenzyl, then R^{X3} is not 4-carboxythiazol-2-yl;
if X³ is a nitrogen atom, X¹, X², X⁴, X⁵, X⁶, X⁷ and X⁸ are carbon atoms, and L is —CO—, then L^{X3} is not —C(R^{LX3})(R^{LX3})—;
if X² and X³ are nitrogen atoms, X¹, X⁴, X⁵, X⁶, X⁷ and X⁸ are carbon atoms, and L is —SO₂—, then L^{X3} is not —CH₂—;
if X², X³ and X⁸ are nitrogen atoms, X¹, X⁴, X⁵, X⁶ and X⁷ are carbon atoms, L is —CO—, R^{X1} is methyl, and R^{X3} is pyridin-2-yl, then ring A is not piperidin-1-yl.

The present invention also relates to a pharmaceutical composition comprising a compound of formula (I), or a pharmaceutically acceptable salt or solvate thereof, in combination with a pharmaceutically acceptable excipient. Accordingly, the invention relates to a compound of formula (I) or a pharmaceutically acceptable salt or solvate thereof, or a pharmaceutical composition comprising any of the aforementioned entities and a pharmaceutically acceptable excipient, for use as a medicament.

The invention further relates to a compound of formula (I) or a pharmaceutically acceptable salt or solvate thereof, or a pharmaceutical composition comprising any of the aforementioned entities and a pharmaceutically acceptable excipient, for use in the treatment or prevention of a PAR-2 mediated disease or disorder. Thus, the invention in particular provides a pharmaceutical composition comprising, as an active ingredient, a compound of formula (I) or a pharmaceutically acceptable salt or solvate thereof, together with a pharmaceutically acceptable excipient, for use in the treatment or prevention of a PAR-2 mediated disease or disorder.

Moreover, the present invention relates to the use of a compound of formula (I) or a pharmaceutically acceptable salt or solvate thereof in the preparation of a medicament for the treatment or prevention of a PAR-2 mediated disease or disorder.

The invention likewise relates to a method of treating or preventing a PAR-2 mediated disease or disorder, the method comprising administering a compound of formula (I) or a pharmaceutically acceptable salt or solvate thereof, or a pharmaceutical composition comprising any of the aforementioned entities in combination with a pharmaceutically acceptable excipient, to a subject (preferably a human) in need thereof. It will be understood that a therapeutically effective amount of the compound of formula (I) or the pharmaceutically acceptable salt or solvate thereof (or of the pharmaceutical composition) is to be administered in accordance with this method.

As explained above, the disease or disorder to be treated or prevented with a compound of formula (I) or a pharmaceutically acceptable salt or solvate thereof (or a corresponding pharmaceutical composition) in accordance with the present invention includes any PAR-2 mediated disease or disorder. It is preferred that the disease/disorder to be treated or prevented in accordance with the invention is pain (e.g., chronic pain), an autoimmune disorder, an autoinflammatory disorder, an inflammatory disorder (e.g., a rheumatologic inflammatory disorder, a skin inflammatory disorder, a lung inflammatory disorder, a muscle inflammatory disorder, a bowel inflammatory disorder, or a brain inflammatory disorder), a central nervous system disorder, spinal cord injury, a metabolic disorder, a gastrointestinal disorder, a cardiovascular disorder, a fibrotic disorder, a respiratory disorder, a skin disorder, an allergic disorder, or cancer. More preferably, the disease/disorder to be treated or prevented in accordance with the present invention is selected from neuropathic pain, inflammatory pain, cancer pain, post-operative incision pain, fracture pain, osteoporotic fracture pain, gout joint pain, chronic pain, spinal cord injury, atopic dermatitis, contact dermatitis, dry skin dermatitis, seborrhoeic dermatitis, arthritis, rheumatoid arthritis, osteoarthritis, psoriasis, psoriatic arthritis, multiple sclerosis, non-alcoholic steatohepatitis (NASH), obesity (e.g., diet-induced obesity), diabetes (e.g., type 1 diabetes or type 2 diabetes), adipose inflammation, pancreatitis, metabolic syndrome, PAR-2 associated metabolic dysfunction, periodontitis, gingivitis, inflammatory bowel disease, Crohn's disease, ulcerative colitis, peptic ulcer disease (e.g., gastric ulcer or duodenal ulcer), infectious enteritis, irritable bowel syndrome, atherosclerosis, asthma, interstitial lung disease, pulmonary fibrosis (e.g., idiopathic pulmonary fibrosis), rheumatoid arthritis-associated interstitial lung disease, liver fibrosis, cystic fibrosis, renal fibrosis, peritoneal fibrosis, pancreatic fibrosis, intestinal fibrosis, cardiac fibrosis, skin fibrosis, systemic lupus erythematosus (SLE), scleroderma, skin eczema, acne, rosacea, post-inflammatory hyperpigmentation, lichen planus, pruritus, polymyositis, vasculitis, Wegener's granulomatosis (or granulomatosis with polyangiitis), Netherton syndrome, dermatomyositis, uveitis, liver cirrhosis, Alzheimer's disease, Parkinson's disease, dust mite allergy (e.g., house dust mite allergy), cockroach allergy, allergic asthma, colorectal cancer, colon cancer (e.g., colon adenocarcinoma), gastric cancer (e.g., stomach adenocarcinoma), rectal cancer (e.g., rectum adenocarcinoma), liver cancer (e.g., hepatocellular carcinoma), breast cancer, pancreatic cancer (e.g., pancreatic adenocarcinoma or pancreatic ductal adenocarcinoma), cervical cancer (e.g., cervical squamous cell carcinoma or endocervical adenocarcinoma), prostate cancer (e.g., prostate adenocarcinoma), ovarian cancer (e.g., ovarian serous cystadenocarcinoma), endometrial cancer (e.g., uterine corpus endometrial carcinoma), uterine sarcoma (e.g., uterine carcinosarcoma), germ cell cancer (e.g., testicular germ cell cancer), esophageal cancer, leukemia (e.g., acute myeloid leukemia), lung cancer (e.g., lung adenocarcinoma or lung squamous cell carcinoma), adrenal gland cancer (e.g., adrenocortical carcinoma), bile duct cancer (e.g., cholangio carcinoma), bladder cancer (e.g., bladder urothelial carcinoma), head and neck cancer, kidney cancer (e.g., kidney chromophobe, kidney renal cell carcinoma, kidney renal clear cell carcinoma, or kidney renal papillary cell carcinoma), lymphoma (e.g., lymphoid neoplasm diffuse large B-cell lymphoma), mesothelioma, sarcoma, melanoma (e.g., skin cutaneous melanoma, or uveal melanoma), thyroid carcinoma, thymus cancer (e.g., thymoma), or glioblastoma.

Accordingly, the present invention particularly relates to a compound of formula (I) or a pharmaceutically acceptable salt or solvate thereof, or a pharmaceutical composition comprising any of the aforementioned entities and a pharmaceutically acceptable excipient, for use in the treatment or prevention of neuropathic pain, inflammatory pain, cancer pain, post-operative incision pain, fracture pain, osteoporotic fracture pain, gout joint pain, chronic pain, spinal cord injury, atopic dermatitis, contact dermatitis, dry skin dermatitis, seborrhoeic dermatitis, arthritis, rheumatoid arthritis, osteoarthritis, psoriasis, psoriatic arthritis, multiple sclerosis, non-alcoholic steatohepatitis (NASH), obesity (e.g., diet-induced obesity), diabetes, adipose inflammation, pancreatitis, metabolic syndrome, PAR-2 associated metabolic dysfunction, periodontitis, gingivitis, inflammatory bowel disease, Crohn's disease, ulcerative colitis, peptic ulcer disease (e.g., gastric ulcer or duodenal ulcer), infectious enteritis, irritable bowel syndrome, atherosclerosis, asthma, interstitial lung disease, pulmonary fibrosis (e.g., idiopathic pulmonary fibrosis), rheumatoid arthritis-associated interstitial lung disease, liver fibrosis, cystic fibrosis, renal fibrosis, peritoneal fibrosis, pancreatic fibrosis, intestinal fibrosis, cardiac fibrosis, skin fibrosis, systemic lupus erythematosus (SLE), scleroderma, skin eczema, acne, rosacea, post-inflammatory hyperpigmentation, lichen planus, pruritus, polymyositis, vasculitis, Wegener's granulomatosis (or granulomatosis with polyangiitis), Netherton syndrome, dermatomyositis, uveitis, liver cirrhosis, Alzheimer's disease, Parkinson's disease, dust mite allergy (e.g., house dust mite allergy), cockroach allergy, allergic asthma, or cancer (e.g., colorectal cancer, colon cancer (e.g., colon adenocarcinoma), gastric cancer (e.g., stomach adenocarcinoma), rectal cancer (e.g., rectum adenocarcinoma), liver cancer (e.g., hepatocellular carcinoma), breast cancer, pancreatic cancer (e.g., pancreatic adenocarcinoma or pancreatic ductal adenocarcinoma), cervical cancer (e.g., cervical squamous cell carcinoma or endocervical adenocarcinoma), prostate cancer (e.g., prostate adenocarcinoma), ovarian cancer (e.g., ovarian serous cystadenocarcinoma), endometrial cancer (e.g., uterine corpus endometrial carcinoma), uterine sarcoma (e.g., uterine carcinosarcoma), germ cell cancer (e.g., testicular germ cell cancer), esophageal cancer, leukemia (e.g., acute myeloid leukemia), lung cancer (e.g., lung adenocarcinoma or lung squamous cell carcinoma), adrenal gland cancer (e.g., adrenocortical carcinoma), bile duct cancer (e.g., cholangio carcinoma), bladder cancer (e.g., bladder urothelial carcinoma), head and neck cancer, kidney cancer (e.g., kidney chromophobe, kidney renal cell carcinoma, kidney renal clear cell carcinoma, or kidney renal papillary cell carcinoma), lymphoma (e.g., lymphoid neoplasm diffuse large B-cell lymphoma), mesothelioma, sarcoma, melanoma (e.g., skin cutaneous melanoma, or uveal melanoma), thyroid carcinoma, thymus cancer (e.g., thymoma), or glioblastoma.

The present invention also relates to a compound of formula (I) or a pharmaceutically acceptable salt or solvate thereof, wherein said compound is conjugated via a linker to a membrane anchor. The corresponding conjugate can be employed in place of the compound of formula (I) for any use or purpose described in the present specification, e.g., for use in the treatment of prevention of a PAR-2 mediated disease or disorder, including any of the diseases/disorders mentioned herein above. Such conjugates are advantageous in that they allow to tether the conjugated compound of formula (I) to a cell membrane in the proximity of PAR-2 and, thus, to facilitate its interaction with PAR-2.

The membrane anchor may be any moiety that is capable of inserting/partitioning into a lipid membrane (preferably a cell membrane), particularly a hydrophobic moiety or a lipid moiety; the conjugated compound of formula (I) is thereby “anchored” to the corresponding lipid membrane. For example, the membrane anchor may be a C₁₂₋₂₀ alkanoyl group (e.g., a hexadecanoyl group, —CO—(CH₂)₁₄—CH₃), cholesterol, cholestanol, a sphingolipid, or glycophosphatidylinositol (GPI). The membrane anchor may also be, e.g., a moiety of formula (II), (III), (VI), (VII), (VIII), (IX), (X), (XI), (XII), (XIII), (XIV) or (XV) as described and defined in WO 2017/197463, particularly on pages 10 to 15 of WO 2017/197463 which is incorporated herein by reference. The membrane anchor may further be, e.g., a raftophile A or A′, or a moiety of any one of the formulae 2, 200a to 200m, 3, 300a to 300g, 4a, 400aa to 400ap, 4b, 400ba, 5a, 500aa to 500ae, 5b, 500ba, 6, 600, 7, 700, 700a to 700c, 8a, 800a, 8b, 9, 900, 10, 1000, 11, 1100a, 1100b, 12, 1200a, 1200b, 13a, 1300aa to 1300ac, 13b, 1300b, 14a, 1400aa to 1400ae, 14b, 1400b, 14c, 15, 1500a, 16, 1600a, 18a, 1800a to 1800d, 18b, 19a, 1900a, 19b or 1900b, as described and defined in WO 2005/097199 which is incorporated herein by reference.

The linker is covalently bound to the membrane anchor and to the compound of formula (I) (or the pharmaceutically acceptable salt or solvate thereof). While the linker is not particularly limited, it preferably has a length of about 1 nm to about 50 nm, and/or it preferably provides a distance of at least 8 atoms between the compound of formula (I) and the membrane anchor. For example, the linker may comprise one or more polyethylene glycol (PEG) units, or may comprise a peptide sequence (which may be composed, e.g., of 2 to 200 amino acid residues). The linker may also be, e.g., a moiety of formula (IV), (XX), (XXI) or (XXII) as described and defined in WO 2017/197463, particularly on pages 15 to 18 of WO 2017/197463 which is incorporated herein by reference. The linker may further be, e.g., a linker B or B′, or a moiety of any one of the formulae 20, 2000, 2001, 21, 2100, 2101, 22, 23, 28 or 28a, as described and defined in WO 2005/097199 which is incorporated herein by reference. It will be understood that the linker may be attached to the membrane anchor via any suitable chemical linkage, e.g. via an amide linkage or via an ester linkage. Likewise, the linker may be attached to the compound of formula (I) (or the pharmaceutically acceptable salt or solvate thereof) via any suitable chemical linkage, e.g. via an amide linkage or via an ester linkage. While the linker may be attached at any position (or to any functional group) of the compound of formula (I) or the pharmaceutically acceptable salt or solvate thereof, it is preferred that the linker is attached to ring A or to a substituent R^{A} on ring A.

Moreover, the linker and the membrane anchor may together form, e.g., any one of the moieties described to be attached to a PAR-2 inhibitor in WO 2017/197463, or to a PAR-2 modulating compound in WO 2017/173347, or to a pharmacophore in WO 2005/097199. Suitable protocols for the preparation of corresponding linkers and membrane anchors are also described in these documents.

An example of a corresponding conjugate, wherein a compound of formula (I) is conjugated via a linker to a membrane anchor, is described in Example 227.

Accordingly, the invention provides a compound of formula (I) or a pharmaceutically acceptable salt or solvate thereof, wherein said compound is conjugated via a linker to a membrane anchor, wherein the membrane anchor is a C₁₂₋₂₀ alkanoyl group (e.g., a hexadecanoyl group, —CO—(CH₂)₁₄—CH₃). The invention particularly provides the compound N-(37-(4-(1-(tert-butyl)-3-(4-chloro-3-fluorophenyl)-1H-pyrrolo[2,3-b]pyridine-6-carbonyl)-3,3-dimethylpiperazin-1-yl)-3-methyl-4,17,30,37-tetraoxo-7,10,13,20,23,26-hexaoxa-3,16,29-triazaheptatriacontyl)-N-methylpalmitamide or a pharmaceutically acceptable salt or solvate thereof.

The present invention furthermore relates to the use of a compound of formula (I) or a pharmaceutically acceptable salt or solvate thereof as an inhibitor of protease-activated receptor 2 (PAR-2) in research, particularly as a research tool compound for inhibiting PAR-2. Accordingly, the invention refers to the in vitro use of a compound of formula (I) or a pharmaceutically acceptable salt or solvate thereof as a PAR-2 inhibitor and, in particular, to the in vitro use of a compound of formula (I) or a pharmaceutically acceptable salt or solvate thereof as a research tool compound acting as a PAR-2 inhibitor. The invention likewise relates to a method, particularly an in vitro method, of inhibiting PAR-2, the method comprising the application of a compound of formula (I) or a pharmaceutically acceptable salt or solvate thereof. The invention further relates to a method of inhibiting PAR-2, the method comprising applying a compound of formula (I) or a pharmaceutically acceptable salt or solvate thereof to a test sample (e.g., a biological sample) or a test animal (i.e., a non-human test animal). The invention also refers to a method, particularly an in vitro method, of inhibiting PAR-2 in a sample (e.g., a biological sample), the method comprising applying a compound of formula (I) or a pharmaceutically acceptable salt or solvate thereof to said sample. The present invention further provides a method of inhibiting PAR-2, the method comprising contacting a test sample (e.g., a biological sample) or a test animal (i.e., a non-human test animal) with a compound of formula (I) or a pharmaceutically acceptable salt or solvate thereof. The terms “sample”, “test sample” and “biological sample” include, without being limited thereto: a cell, a cell culture or a cellular or subcellular extract; biopsied material obtained from an animal (e.g., a human), or an extract thereof; or blood, serum, plasma, saliva, urine, feces, or any other body fluid, or an extract thereof. It is to be understood that the term “in vitro” is used in this specific context in the sense of “outside a living human or animal body”, which includes, in particular, experiments performed with cells, cellular or subcellular extracts, and/or biological molecules in an artificial environment such as an aqueous solution or a culture medium which may be provided, e.g., in a flask, a test tube, a Petri dish, a microtiter plate, etc.

The compounds of formula (I) as well as the pharmaceutically acceptable salts and solvates thereof will be described in more detail in the following.

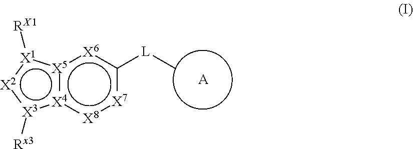

As depicted in formula (I), the bicyclic ring system containing the ring atoms X¹ to X⁸ is aromatic. In particular, each ring of this bicyclic ring system is aromatic.

One, two, three or four of the ring atoms X¹ to X⁸ are nitrogen atoms, and all remaining ring atoms (among X¹ to X⁸) are carbon atoms. Any among the ring atoms X², X⁶, X⁷ and X⁸ that is a carbon atom is optionally substituted with a group R^{X}.

It will be understood that the position of the nitrogen ring atom(s) within the bicyclic ring system containing X¹ to X⁸ is limited by the requirement that this bicyclic ring system is aromatic and by the requirement that a group R^{X1} is attached to the ring atom X¹ and a group R^{X3} is attached to the ring atom X³. Thus, for example, while X¹ and X³ may each be a nitrogen atom or a carbon atom, they cannot both be a nitrogen atom.

It is preferred that two, three or four of the ring atoms X¹ to X⁸ are nitrogen atoms, and all remaining ring atoms X¹ to X⁸ are carbon atoms. It is particularly preferred that two, three or four of the ring atoms X¹ to X⁸ are nitrogen atoms, wherein X⁶ is a nitrogen atom, wherein one, two or three ring atoms selected from X¹, X², X³, X⁴, X⁵, X⁷ and X⁸ are nitrogen atoms, and wherein all remaining ring atoms are carbon atoms. Accordingly, in a preferred embodiment, X¹ and X⁶ are nitrogen atoms, and all remaining ring atoms (i.e., X², X³, X⁴, X⁵, X⁷ and X⁸) are carbon atoms. In a further preferred embodiment, X¹, X² and X⁶ are nitrogen atoms, and all remaining ring atoms (i.e., X³, X⁴, X⁵, X⁷ and X⁸) are carbon atoms. In a further preferred embodiment, X², X³ and X⁶ are nitrogen atoms, X¹, X⁴ and X⁵ are carbon atoms, one of X⁷ and X⁸ is a carbon atom or a nitrogen atom, and the other one of X⁷ and X⁸ is a carbon atom. In a further preferred embodiment, X², X³ and X⁶ are nitrogen atoms, and all remaining ring atoms (i.e., X¹, X⁴, X⁵, X⁷ and X⁸) are carbon atoms. As explained above, any among the ring atoms X², X⁶, X⁷ and X⁸ that is a carbon atom is optionally substituted with a group R^{X}.

For example, the bicyclic ring system

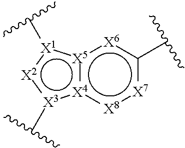

may be selected from any one of the following groups:

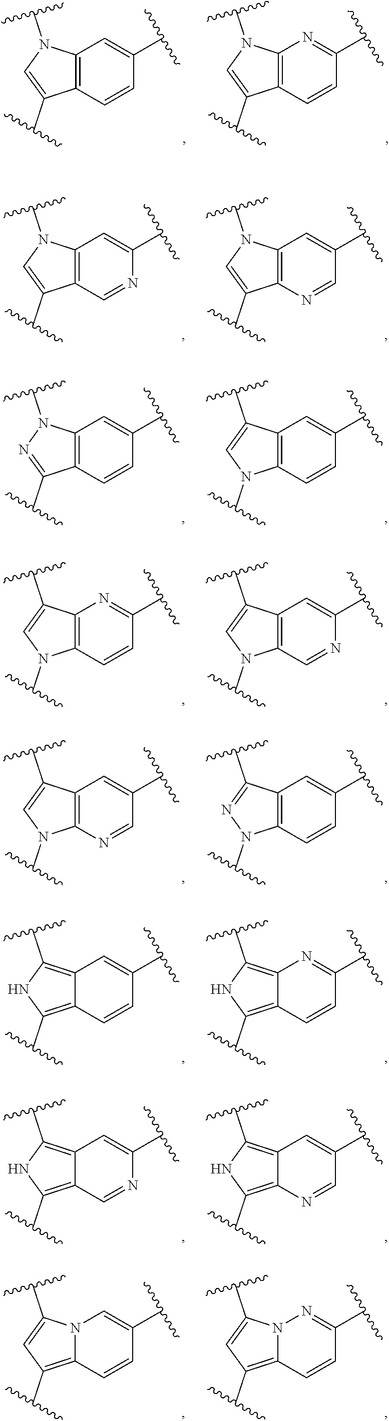

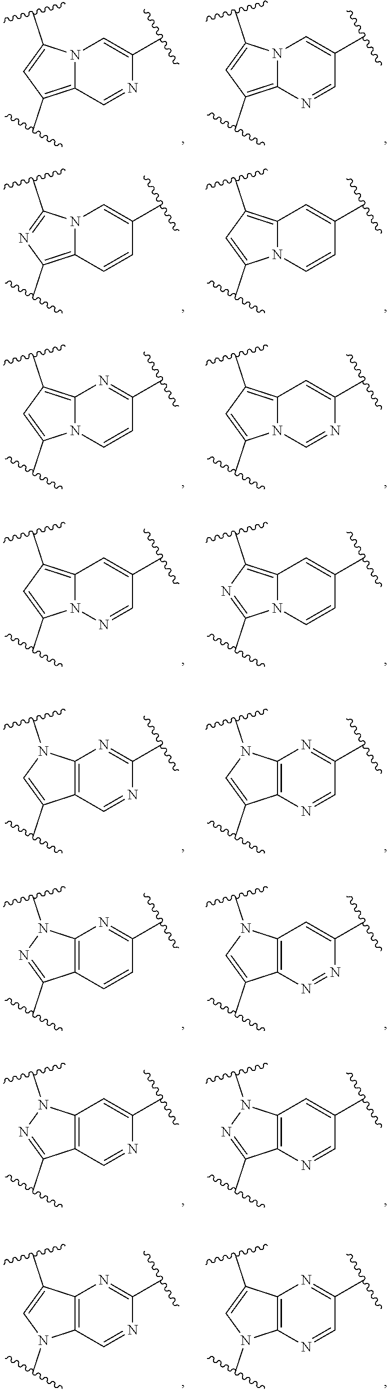

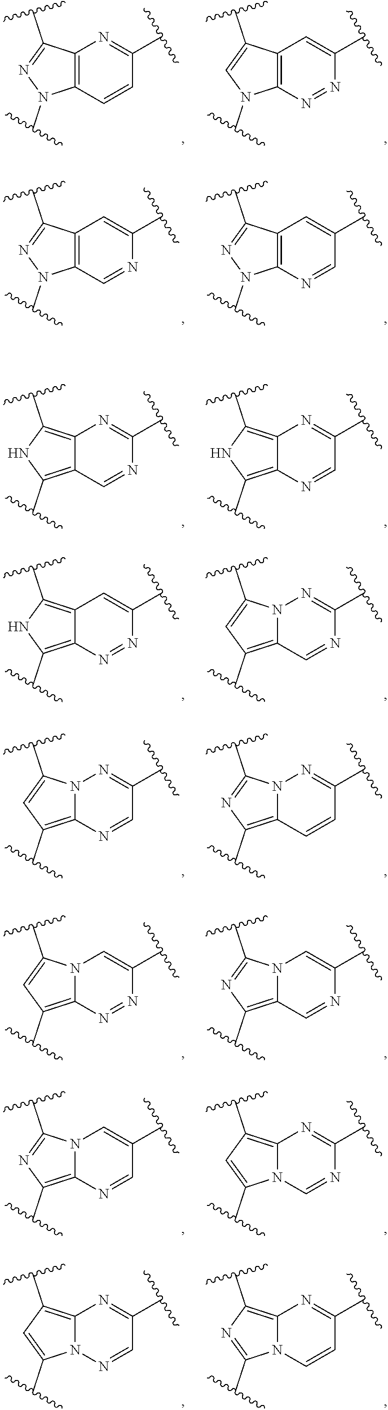

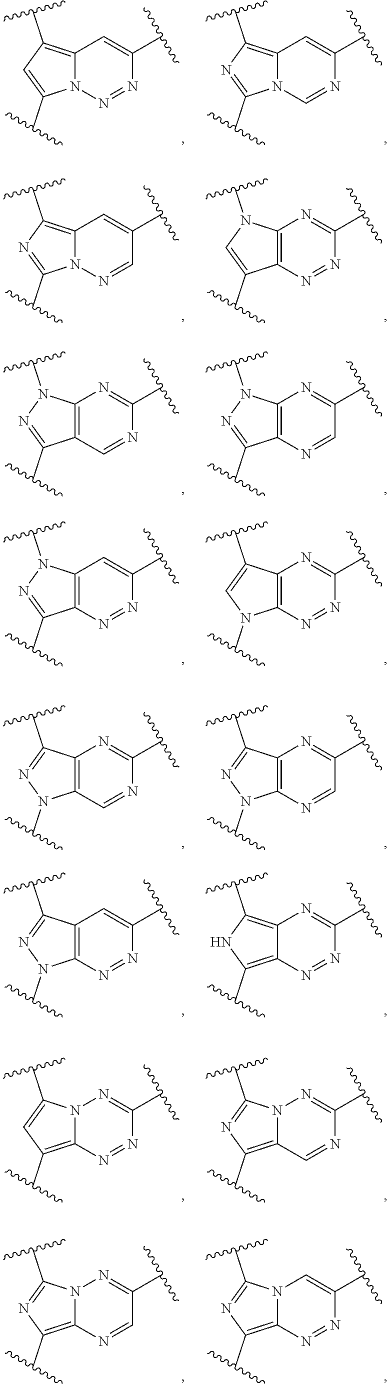

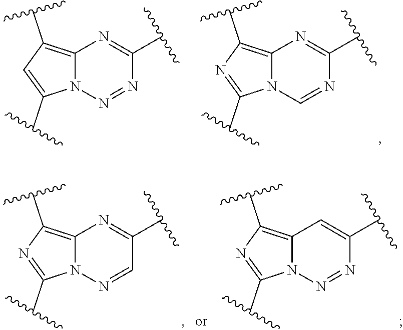 [SMILES: *c1nnn2c(*)cc(*)c2n1 ; *c1ncn2c(*)nc(*)c2n1 ; *c1cnn2c(*)nc(*)c2n1 ; *c1cc2c(*)nc(*)n2nn1]

wherein each of the above-depicted groups is optionally substituted with one or more groups R^{X}.

In particular, the bicyclic ring system

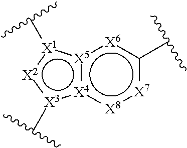

may be selected from any one of the following groups:

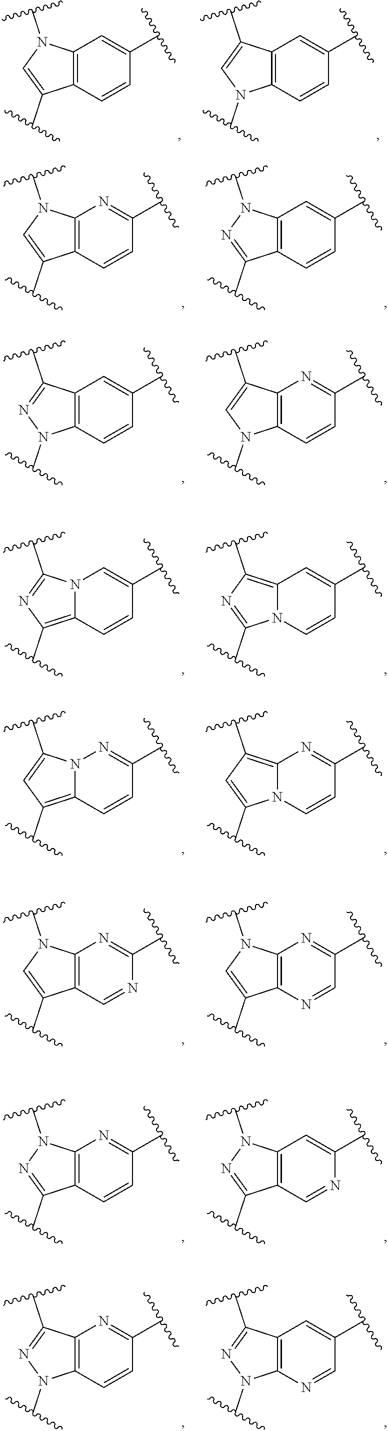

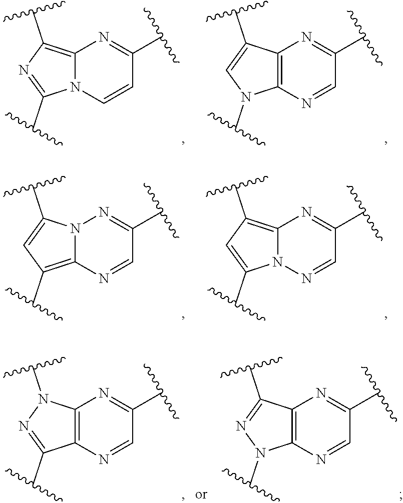 [SMILES: *c1ccn2c(*)nc(*)c2n1 ; *c1cnc2c(n1)c(*)cn2* ; *c1cnc2c(*)cc(*)n2n1 ; *c1cnn2c(*)cc(*)c2n1 ; *c1cnc2c(*)nn(*)c2n1 ; *c1cnc2c(n1)c(*)nn2*]

wherein each of the above-depicted groups is optionally substituted with one or more groups R^{X}.

More specifically, the bicyclic ring system

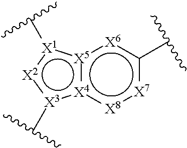

may be selected from any one of the following groups:

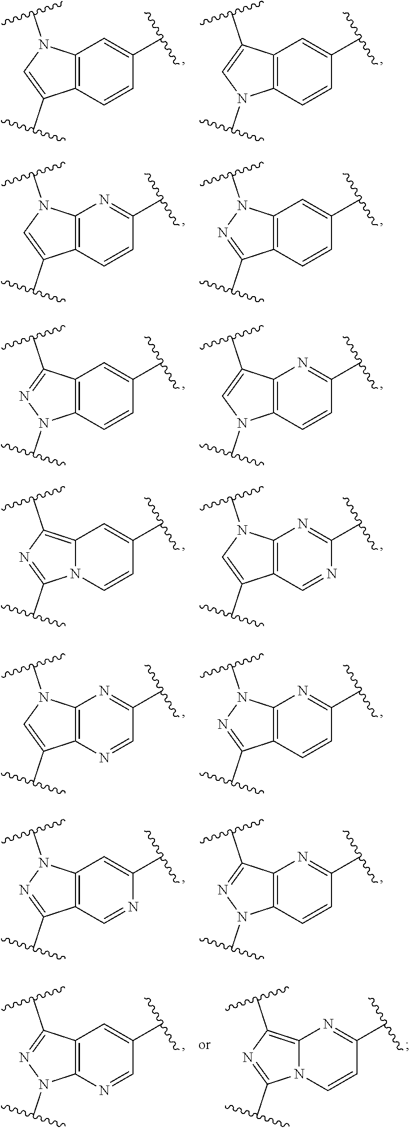

wherein each of the above-depicted groups is optionally substituted with one or more groups R^{X}.

As explained above, it is preferred that two, three or four of the ring atoms X¹ to X⁸ are remaining ring atoms X¹ to X⁸ are carbon atoms. Accordingly, the bicyclic ring system

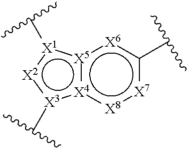

is preferably selected from any one of the following groups:

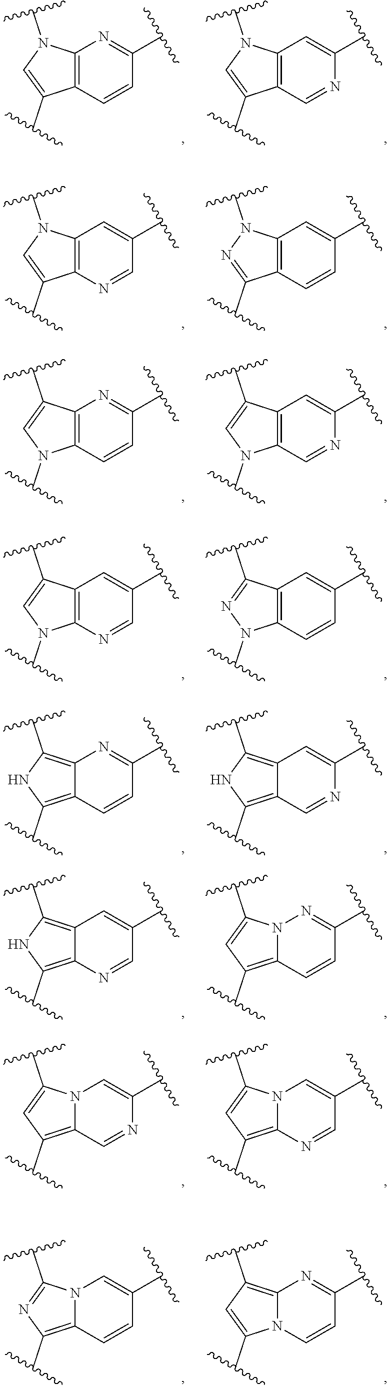

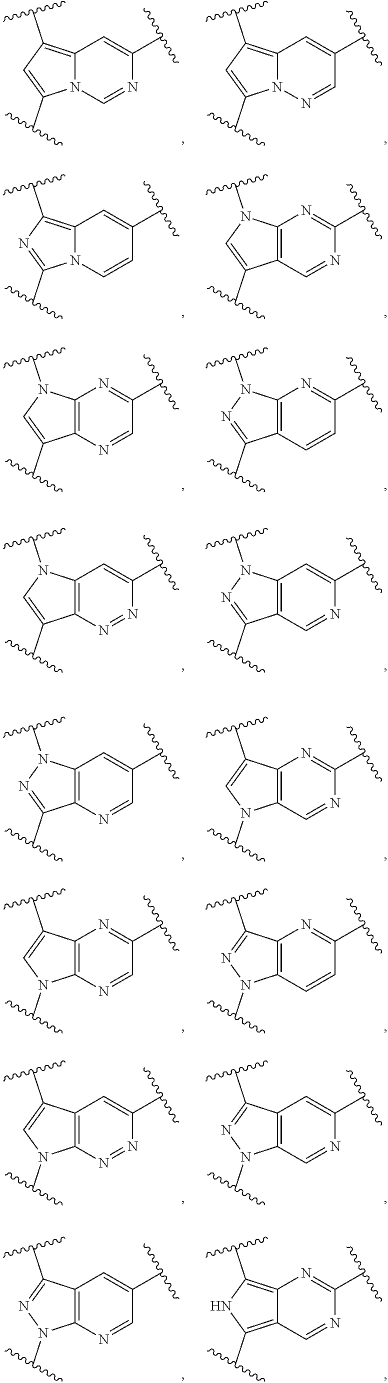

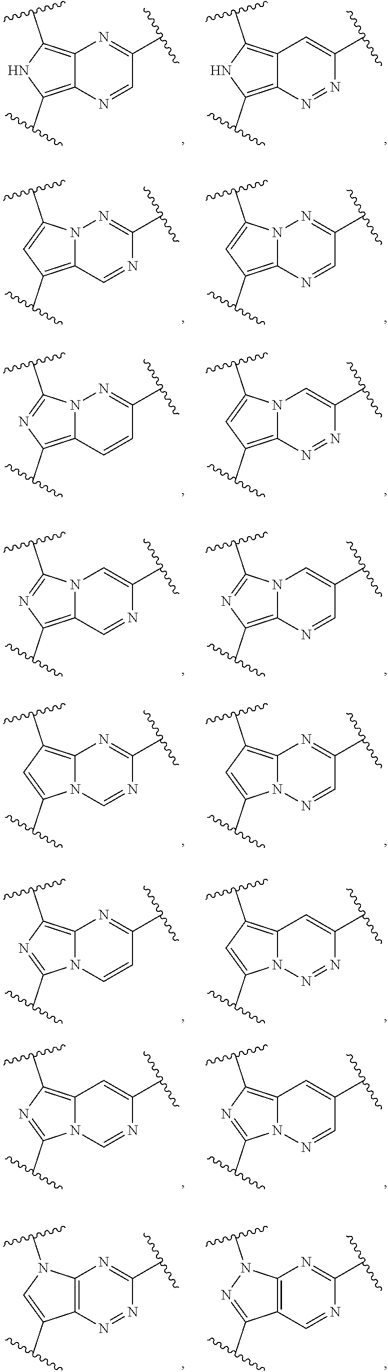

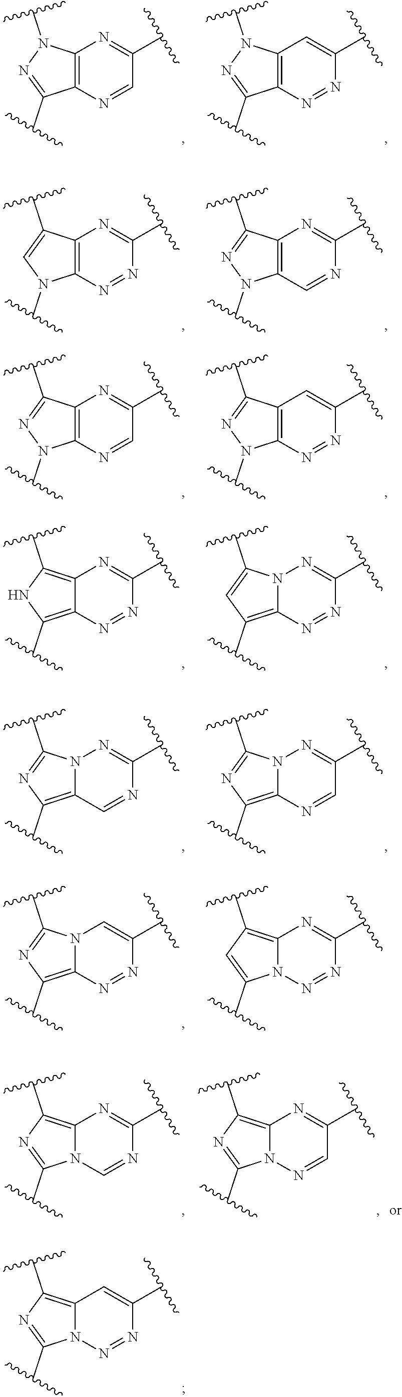

wherein each of the above-depicted groups is optionally substituted with one or more groups R^{X}.

As also explained above, it is particularly preferred that two, three or four of the ring atoms X¹ to X⁸ are nitrogen atoms, wherein X⁶ is a nitrogen atom, wherein one, two or three ring atoms selected from X¹, X², X³, X⁴, X⁵, X⁷ and X⁸ are nitrogen atoms, and wherein all remaining ring atoms are carbon atoms. Accordingly, the bicyclic ring system

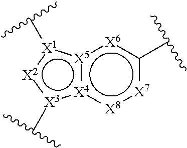

is more preferably selected from any one of the following groups:

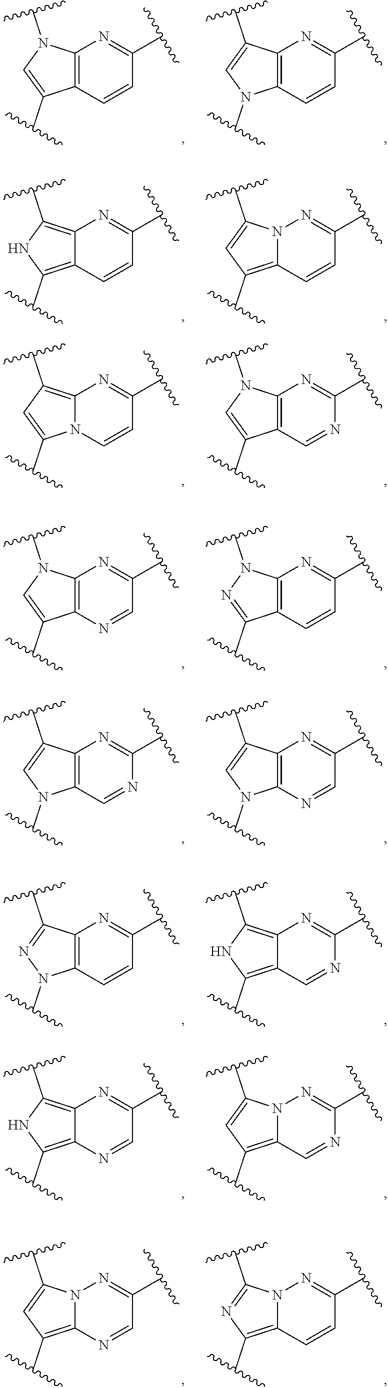

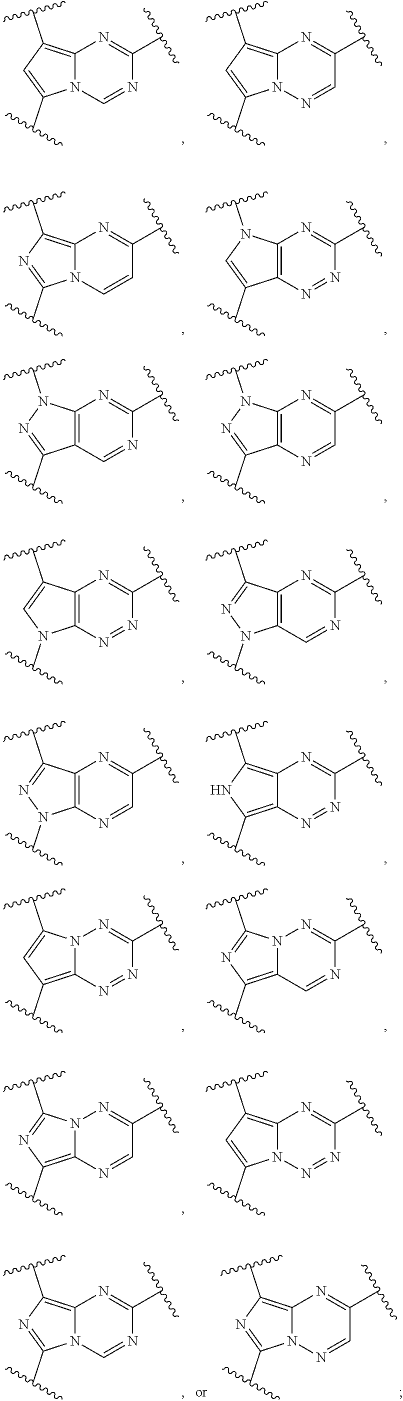

wherein each of the above-depicted groups is optionally substituted with one or more groups R^{X}.

Even more preferably, the bicyclic ring system

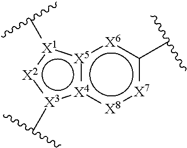

is selected from any one of the following groups:

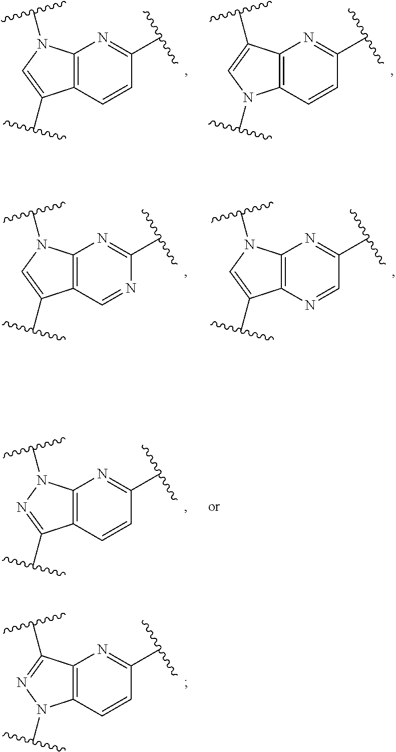 [SMILES: *c1ccc2c(*)cn(*)c2n1 ; *c1ccc2c(n1)c(*)cn2* ; *c1ncc2c(*)cn(*)c2n1 ; *c1cnc2c(*)cn(*)c2n1 ; *c1ccc2c(*)nn(*)c2n1 ; *c1ccc2c(n1)c(*)nn2*]

wherein each of the above-depicted groups is optionally substituted with one or more groups R^{X}.

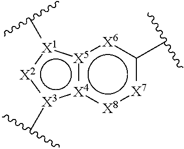

Yet even more preferably, the bicyclic ring system is selected from any one of the following groups:

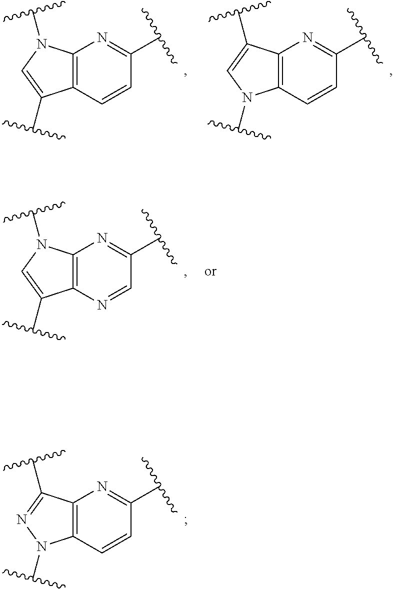 [SMILES: *c1ccc2c(*)cn(*)c2n1 ; *c1ccc2c(n1)c(*)cn2* ; *c1cnc2c(*)cn(*)c2n1 ; *c1ccc2c(n1)c(*)nn2*]

wherein each of the above-depicted groups is optionally substituted with one or more (e.g., one or two) groups R^{X}; preferably wherein each of the above-depicted groups is not substituted with any R^{X}.

It is particularly preferred that the bicyclic ring system

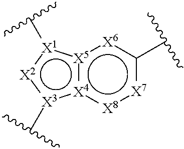

is a group

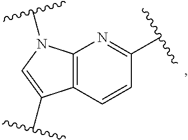 [SMILES: *c1ccc2c(*)cn(*)c2n1]

wherein said group is optionally substituted with one or more (e.g., one or two) groups R^{X}, preferably wherein said group is not substituted with any R^{X}.

As explained above, any among the ring atoms X², X⁶, X⁷ and X⁸ that is a carbon atom is optionally substituted with a group R^{X}. Conversely, if any of X², X⁶, X⁷ and X⁸ is a nitrogen atom, the corresponding nitrogen atom does not carry any optional substituent. Likewise, the ring atoms X⁴ and X⁵ do not carry any optional substituent. It will be understood that if any of X², X⁶, X⁷ and X⁸ is a carbon atom which is substituted with a group R^{X}, the group R^{X} replaces a hydrogen atom that would otherwise (i.e., in the absence of the optional substituent R^{X}) be attached to the respective carbon atom. It is preferred that the compound of formula (I) comprises zero, one or two groups R^{X}, more preferably zero or one group R^{X}, even more preferably zero (i.e., no) groups R^{X}. If the compound of formula (I) comprises one group R^{X}, the corresponding group R^{X} may be present, in particular, at a ring carbon atom in position X².

Each R^{X} is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-NH—OH, —(C₀₋₃ alkylene)-N(C₁₋₅ alkyl)-OH, —(C₀₋₃ alkylene)-NH—O(C₁₋₅ alkyl), —(C₀₋₃ alkylene)-N(C₁₋₅ alkyl)-O(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-NH—COO(C₁₋₅ alkyl), —(C₀₋₃ alkylene)-N(C₁₋₅ alkyl)-COO(C₁₋₅ alkyl), —(C₀₋₃ alkylene)-O—CO—NH(C₁₋₅ alkyl), —(C₀₋₃ alkylene)-O—CO—N(C₁₋₅ alkyl)(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-SO—(C₁₋₅ alkyl), —(C₀₋₃ alkylene)-SO₂—(C₁₋₅ alkyl), —(C₀₋₃ alkylene)-carbocyclyl, —(C₀₋₃ alkylene)-heterocyclyl, and -L¹-R^{L1}, wherein the carbocyclyl group in said —(C₀₋₃ alkylene)-carbocyclyl and the heterocyclyl group in said —(C₀₋₃ alkylene)-heterocyclyl are each optionally substituted with one or more (e.g., one, two or three) groups R^{Cyc}. As explained above, each R^{X} is attached to a carbon ring atom of the bicyclic ring system.

Preferably, each R^{X} is independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O—(C₁₋₅ haloalkyl), —CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO—(C₁₋₅ alkyl), —SO₂—(C₁₋₅ alkyl), —(C₀₋₃ alkylene)-carbocyclyl, —(C₀₋₃ alkylene)-heterocyclyl, and -L¹-R^{L1}, wherein the carbocyclyl group in said —(C₀₋₃ alkylene)-carbocyclyl and the heterocyclyl group in said —(C₀₋₃ alkylene)-heterocyclyl are each optionally substituted with one or more groups R^{Cyc}. More preferably, each R^{X} is independently selected from C₁₋₅ alkyl, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), —CN, —(C₀₋₃ alkylene)-cycloalkyl (e.g., cyclopropyl), and —(C₀₋₃ alkylene)-heterocycloalkyl, wherein the cycloalkyl group in said —(C₀₋₃ alkylene)-cycloalkyl and the heterocycloalkyl group in said —(C₀₋₃ alkylene)-heterocycloalkyl are each optionally substituted with one or more groups R^{Cyc}.

R^{X1} is selected from C₁₋₅ alkyl, C₂₋₅ alkenyl, C₂₋₅ alkynyl, —(C₀₋₅ alkylene)-carbocyclyl, and —(C₀₋₅ alkylene)-heterocyclyl, wherein said alkyl, said alkenyl, said alkynyl, the alkylene group in said —(C₀₋₅ alkylene)-carbocyclyl, and the alkylene group in said —(C₀₋₅ alkylene)-heterocyclyl are each optionally substituted with one or more (e.g., one, two, or three) groups R^{X11}, wherein one or more (e.g., one, two, or three) —CH₂— units comprised in said alkyl, said alkenyl, said alkynyl, in the alkylene group in said —(C₀₋₅ alkylene)-carbocyclyl, or in the alkylene group in said —(C₀₋₅ alkylene)-heterocyclyl are each optionally replaced by a group independently selected from —O—, —NH—, —N(C₁₋₅ alkyl)-, —CO—, —S—, —SO—, and —SO₂—, and further wherein the carbocyclyl group in said —(C₀₋₅ alkylene)-carbocyclyl and the heterocyclyl group in said —(C₀₋₅ alkylene)-heterocyclyl are each optionally substituted with one or more (e.g., one, two, or three) groups R^{Cyc} Preferably, R^{X1} is selected from C₁₋₅ alkyl, C₂₋₅ alkenyl, C₂₋₅ alkynyl, —(C₀₋₅ alkylene)-carbocyclyl, and —(C₀₋₅ alkylene)-heterocyclyl, wherein said alkyl, said alkenyl, said alkynyl, the alkylene group in said —(C₀₋₅ alkylene)-carbocyclyl, and the alkylene group in said —(C₀₋₅ alkylene)-heterocyclyl are each optionally substituted with one or more (e.g., one, two, or three) groups independently selected from —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), and —CN, wherein one or more (e.g., one, two, or three) —CH₂— units comprised in said alkyl, said alkenyl, said alkynyl, in the alkylene group in said —(C₀₋₅ alkylene)-carbocyclyl, or in the alkylene group in said —(C₀₋₅ alkylene)-heterocyclyl are each optionally replaced by a group independently selected from —O—, —NH—, —N(C₁₋₅ alkyl)-, —CO—, —S—, —SO—, and —SO₂—, and further wherein the carbocyclyl group in said —(C₀₋₅ alkylene)-carbocyclyl and the heterocyclyl group in said —(C₀₋₅ alkylene)-heterocyclyl are each optionally substituted with one or more (e.g., one, two, or three) groups R^{Cyc}. More preferably, R^{X1} is selected from C₁₋₅ alkyl, —(C₀₋₅ alkylene)-cycloalkyl, —(C₀₋₅ alkylene)-aryl, —(C₀₋₅ alkylene)-heterocycloalkyl, and —(C₀₋₅ alkylene)-heteroaryl, wherein said alkyl or the alkylene group in any of said —(C₀₋₅ alkylene)-cycloalkyl, said —(C₀₋₅ alkylene)-aryl, said —(C₀₋₅ alkylene)-heterocycloalkyl, or said —(C₀₋₅ alkylene)-heteroaryl is optionally substituted with one or more groups independently selected from —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), and —CN, wherein one or more —CH₂— units comprised in said alkyl or in the alkylene group in any of said —(C₀₋₅ alkylene)-cycloalkyl, said —(C₀₋₅ alkylene)-aryl, said —(C₀₋₅ alkylene)-heterocycloalkyl, or said —(C₀₋₅ alkylene)-heteroaryl are each optionally replaced by a group independently selected from —O—, —NH—, —N(C₁₋₅ alkyl)-, —CO—, —S—, —SO—, and —SO₂—, and further wherein the cycloalkyl group in said —(C₀₋₅ alkylene)-cycloalkyl, the aryl group in said —(C₀₋₅ alkylene)-aryl, the heterocycloalkyl group in said —(C₀₋₅ alkylene)-heterocycloalkyl, and the heteroaryl group in said —(C₀₋₅ alkylene)-heteroaryl are each optionally substituted with one or more groups R^{Cyc}. Even more preferably, R^{X1} is selected from C₁₋₅ alkyl, —(C₀₋₅ alkylene)-cycloalkyl, —(C₀₋₅ alkylene)-aryl (e.g., —(C₀₋₅ alkylene)-phenyl, such as —CH₂-phenyl), —(C₀₋₅ alkylene)-heterocycloalkyl, and —(C₀₋₅ alkylene)-heteroaryl, wherein said alkyl or the alkylene group in any of said —(C₀₋₅ alkylene)-cycloalkyl, said —(C₀₋₅ alkylene)-aryl, said —(C₀₋₅ alkylene)-heterocycloalkyl, or said —(C₀₋₅ alkylene)-heteroaryl is optionally substituted with one or more groups independently selected from —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), and —CN, and further wherein the cycloalkyl group in said —(C₀₋₅ alkylene)-cycloalkyl, the aryl group in said —(C₀₋₅ alkylene)-aryl, the heterocycloalkyl group in said —(C₀₋₅ alkylene)-heterocycloalkyl, and the heteroaryl group in said —(C₀₋₅ alkylene)-heteroaryl are each optionally substituted with one or more groups independently selected from C₁₋₅ alkyl, C₂₋₅ alkenyl, C₂₋₅ alkynyl, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), and —CN. Yet even more preferably, R^{X1} is C₁₋₅ alkyl, —(C₀₋₃ alkylene)-cycloalkyl (e.g., cyclopropyl, —CH₂-cyclopropyl, cyclobutyl, —CH₂-cyclobutyl, cyclopentyl, or —CH₂-cyclopentyl), —(C₀₋₃ alkylene)-heterocycloalkyl [e.g., oxetanyl (such as oxetan-2-yl or oxetan-3-yl), —CH₂-oxetanyl (such as oxetan-2-ylmethyl or oxetan-3-ylmethyl), tetrahydrofuranyl (such as tetrahydrofuran-3-yl), —CH₂— tetrahydrofuranyl (such as tetrahydrofuran-3-ylmethyl), tetrahydropyranyl (such as tetrahydropyran-4-yl), or —CH₂— tetrahydropyranyl (such as tetrahydropyran-4-ylmethyl)], or —(C₀₋₃ alkylene)-heteroaryl [e.g., —CH₂-oxazolyl (such as oxazol-2-ylmethyl, oxazol-4-ylmethyl, or oxazol-5-ylmethyl), —CH₂-pyridinyl (such as pyridin-2-ylmethyl, pyridin-3-ylmethyl, or pyridin-4-ylmethyl), —C(—CH₃)(—CH₃)-pyridinyl (such as —C(—CH₃)(—CH₃)-(pyridin-2-yl)), —CH₂-pyrimidinyl (such as pyrimidin-2-ylmethyl), —CH₂-pyrazinyl (such as pyrazin-2-ylmethyl)], wherein said alkyl is optionally substituted with one or more groups independently selected from —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), and —CN, and further wherein the cycloalkyl group in said —(C₀₋₃ alkylene)-cycloalkyl, the heterocycloalkyl group in said —(C₀₋₃ alkylene)-heterocycloalkyl and the heteroaryl in said —(C₀₋₃ alkylene)-heteroaryl are each optionally substituted with one or more groups independently selected from C₁₋₅ alkyl, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), and —CN. Thus, for example, R^{X1} may be C₁₋₅ alkyl which is optionally substituted with one or more groups independently selected from —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), and —CN; a corresponding preferred example of R^{X1} is C₁₋₅ alkyl (e.g., tert-butyl) substituted with one or two groups —O(C₁₋₅ alkyl), such as, e.g., —C(—CH₃)(—CH₃)—CH₂—O—CH₃, —C(—CH₃)(—CH₃)—CH₂—O—CH₂—CH₃, —CH(—CH₂—O—CH₃)(—CH₂—O—CH₃), —CH(—CH₃)—CH₂—O—CH₃, —CH₂CH₂—O—CH₃, or —CH₂CH₂—O—CH₂CH₃. Particularly preferred examples of R^{X1} include methyl, ethyl, isopropyl, iso-butyl, sec-butyl (e.g., (S)-sec-butyl or (R)-sec-butyl), tert-butyl, cyclopropylmethyl, 1-methylcyclobutyl, 3-(methoxymethyl)cyclobutylmethyl, 2,2,2-trifluoroethyl, —C(—CH₃)(—CH₃)—CH₂—O—CH₃, —C(—CH₃)(—CH₃)—CH₂—O—CH₂—CH₃, —CH(—CH₃)—CH₂—O—CH₃ (e.g., (S)—CH(—CH₃)—CH₂—O—CH₃ or (R)—CH(—CH₃)—CH₂—O—CH₃), —CH₂CH₂—O—CH₃, —CH(—CH₂—O—CH₃)(—CH₂—O—CH₃), —CH₂-(cyclobut-1,3-diyl)-CH₂—O—CH₃, pyridin-2- ylmethyl, 6-methoxypyridin-2-ylmethyl, or 1-methyl-1-(pyridin-2-yl)ethyl. Still more preferably, R^{X1} is C₁₋₅ alkyl (e.g., methyl, ethyl, isopropyl, iso-butyl, sec-butyl, or tert-butyl). An especially preferred example of R^{X1} is tert-butyl.

Each R^{X11} is independently selected from —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O—(C₁₋₅ haloalkyl), —CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO—(C₁₋₅ alkyl), —SO₂—(C_{1- 5} alkyl), carbocyclyl, heterocyclyl, and -L¹-R^{L1}, wherein said carbocyclyl and said heterocyclyl are each optionally substituted with one or more (e.g., one, two, or three) groups R^{Cyc}.

Preferably, each R^{X11} is independently selected from —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O—(C₁₋₅ haloalkyl), —CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO—(C₁₋₅ alkyl), and —SO₂—(C₁₋₅ alkyl). More preferably, each R^{X11} is independently selected from —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), and —CN.

R^{X3} is -L^{X3}-carbocyclyl or -L^{X3}-heterocyclyl, wherein the carbocyclyl in said -L^{X3}-carbocyclyl or the heterocyclyl in said -L^{X3}-heterocyclyl is optionally substituted with one or more (e.g., one, two, three, or four) groups R^{X31}.

For example, R^{X3} may be -L^{X3}-aryl, -L^{X3}-cycloalkyl, -L^{X3}-cycloalkenyl, -L^{X3}-heteroaryl, -L^{X3}-heterocycloalkyl, or -L^{X3}-heterocycloalkenyl, wherein the cyclic moiety in each of the aforementioned groups is optionally substituted with one or more groups R^{X31}. Preferably, R^{X3} is -L^{X3}-aryl, -L^{X3}-cycloalkyl or -L^{X3}-heteroaryl, wherein the aryl in said -L^{X3}-aryl, the cycloalkyl in said -L^{X3}-cycloalkyl or the heteroaryl in said -L^{X3}-heteroaryl is optionally substituted with one or more groups R^{X31}. In particular, it is preferred that R^{X3} is selected from -L^{X3}-phenyl, -L^{X3}-naphthyl (e.g., -L^{X3}-naphthalen-1-yl or -L^{X3}-naphthalen-2-yl), -L^{X3}-(C₃₋₇ cycloalkyl), -L^{X3}-(monocyclic 5- or 6-membered heteroaryl), or -L^{X3}-(bicyclic 9- or 10-membered heteroaryl), wherein the cyclic moiety in each of the aforementioned groups is optionally substituted with one or more groups R^{X31}. By way of example, if R^{X3} is -L^{X3}-heteroaryl [e.g., -L^{X3}-(monocyclic 5- or 6-membered heteroaryl) or -L^{X3}-(bicyclic 9- or 10-membered heteroaryl)] wherein the heteroaryl in said -L^{X3}-heteroaryl is optionally substituted with one or more groups R^{X31}, then the heteroaryl in said -L^{X3}-heteroaryl may be, e.g., selected from pyrrolyl (e.g., 1H-pyrrol-1-yl, 1H-pyrrol-2-yl, or 1H-pyrrol-3-yl), pyrazolyl (e.g., pyrazol-1-yl, pyrazol-3-yl, or pyrazol-4-yl), imidazolyl (e.g., imidazol-1-yl, imidazol-2-yl, or imidazol-4-yl), triazolyl (e.g., 1H-1,2,3-triazolyl, 2H-1,2,3-triazolyl, 1H-1,2,4-triazolyl, or 4H-1,2,4-triazolyl; such as, e.g., 1H-1,2,3-triazol-1-yl, 1H-1,2,3-triazol-4-yl, 1H-1,2,3-triazol-5-yl, 1H-1,2,4-triazol-1-yl, 1H-1,2,4-triazol-3-yl, or 1H-1,2,4-triazol-5-yl), furanyl (e.g., furan-2-yl or furan-3-yl), thiophenyl (e.g., thiophen-2-yl or thiophen-3-yl), oxazolyl (e.g., oxazol-2-yl, oxazol-4-yl, or oxazol-5-yl), isoxazolyl (e.g., isoxazol-3-yl, isoxazol-4-yl, or isoxazol-5-yl), thiazolyl, isothiazolyl, pyridinyl (e.g., pyridin-2-yl, pyridin-3-yl, or pyridin-4-yl), pyridazinyl, pyrimidinyl, pyrazinyl, 1H-indolyl, 2H-isoindolyl, indolizinyl (e.g., indolizin-1-yl or indolizin-2-yl), 1H-indazolyl, benzimidazolyl, benzofuranyl (e.g., benzofuran-2-yl, benzofuran-3-yl, benzofuran-4-yl, benzofuran-5-yl, benzofuran-6-yl, or benzofuran-7-yl), isobenzofuranyl, benzo[b]thiophenyl (e.g., benzo[b]thiophen-2-yl, benzo[b]thiophen-3-yl, benzo[b]thiophen-4-yl, benzo[b]thiophen-5-yl, benzo[b]thiophen-6-yl, or benzo[b]thiophen-7-yl), benzo[c]thiophenyl, quinolinyl, isoquinolinyl, quinoxalinyl, phthalazinyl, quinazolinyl, and cinnolinyl. If R^{X3} is -L^{X3}-cycloalkyl [e.g., -L^{X3}-(C₃₋₇ cycloalkyl)] wherein the cycloalkyl in said -L^{X3}-cycloalkyl is optionally substituted with one or more groups R^{X31}, then the cycloalkyl in said -L^{X3}-cycloalkyl may be, e.g., selected from cyclopropyl, cyclobutyl, cyclopentyl, cyclohexyl, or cycloheptyl. Even more preferably, R^{X3} is -L^{X3}-phenyl, wherein the phenyl in said -L^{X3}-phenyl is optionally substituted with one or more (e.g., one, two, or three) groups R^{X31}.

L^{X3} is independently selected from a bond, —C(R^{LX3})(R^{LX3})—, —O—, —S—, —SO—, —SO₂—, —CO—, and —N(R^{LX3})—, wherein each R^{LX3} is independently hydrogen or C₁₋₅ alkyl, and further wherein two groups R^{LX3} which are attached to the same carbon atom may also be mutually joined to form, together with the carbon atom that they are attached to, a cycloalkyl (e.g., a C₃₋₆ cycloalkyl) or a heterocycloalkyl (e.g., a 3 to 6-membered heterocycloalkyl).

Preferably, L^{X3} is independently selected from a bond, —CH₂—, —CH(C₁₋₅ alkyl)-, —C(C₁₋₅ alkyl)(C₁₋₅ alkyl)-, C₃₋₆ cycloalkyl-1,1-ene, —O—, —S—, —SO—, —SO₂—, —CO—, —NH—, and —N(C₁₋₅ alkyl)-. More preferably, L^{X3} is independently selected from a bond, —CH₂—, —CH(C₁₋₅ alkyl)-, —C(C₁₋₅ alkyl)(C₁₋₅ alkyl)-, and C₃₋₅ cycloalkyl-1,1-ene (e.g., cyclopropyl-1,1-ene). Even more preferably, L^{X3} is independently selected from a bond, —CH₂—, —CH(C₁₋₃ alkyl)-, and —C(C₁₋₃ alkyl)(C₁₋₃ alkyl)-. Yet even more preferably, L^{X3} is a bond.

In accordance with the above definition of L^{X3}, it is particularly preferred that R^{X3} is phenyl which is optionally substituted with one or more (e.g., one, two, or three) groups R^{X31}. If said phenyl is optionally substituted with one group R^{X31}, it is preferred that said group R^{X31} is attached in ortho or para position on the phenyl (preferably in para position), i.e., that R^{X3} is 3-R^{X31}-phenyl or 4-R^{X31}-phenyl (preferably 4-R^{X31}-phenyl). If said phenyl is optionally substituted with two groups R^{X31}, it is preferred that the two groups R^{X31} are attached in ortho and para position, i.e., that R^{X3} is 3-R^{X31}-4-R^{X31}-phenyl. If said phenyl is optionally substituted with three groups R^{X31}, it is preferred that two of the three groups R^{X31} are attached in ortho position and one group R^{X31} is attached in para position, i.e., that R^{X3} is 3-R^{X31}-4-R^{X31}-5-R^{X31}-phenyl. It is furthermore preferred that said phenyl is substituted with two or three (particularly with two) groups R^{X31}. Accordingly, it is particularly preferred that R^{X3} is 3-R^{X31}-4-R^{X31}-phenyl or 3-R^{X31}-4-R^{X31}-5-R^{X31}-phenyl, wherein each R^{X31} is independently selected from halogen (e.g., —F, —Cl, —Br, or —I), C₁₋₅ haloalkyl (e.g., —CF₃), and C₁₋₅ alkyl (e.g., —CH₃), even more preferably wherein each R^{X31} is independently selected from —F, —Cl, —CF₃, and —CH₃. Corresponding preferred examples of R^{X3} include 4-chloro-3-fluoro-phenyl, 3,4-dichloro-phenyl, 3,4-difluoro-phenyl, 3-fluoro-4-trifluoromethyl-phenyl, 3-fluoro-4-methyl-phenyl, or 3,4,5-trifluoro-phenyl. A particularly preferred example of R^{X3} is 4-chloro-3-fluoro-phenyl.

Each R^{X31} is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-NH—OH, —(C₀₋₃ alkylene)-N(C₁₋₅ alkyl)-OH, —(C₀₋₃ alkylene)-NH—O(C₁₋₅ alkyl), —(C₀₋₃ alkylene)-N(C₁₋₅ alkyl)-O(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-NH—COO(C₁₋₅ alkyl), —(C₀₋₃ alkylene)-N(C₁₋₅ alkyl)-COO(C₁₋₅ alkyl), —(C₀₋₃ alkylene)-O—CO—NH(C₁₋₅ alkyl), —(C₀₋₃ alkylene)-O—CO—N(C₁₋₅ alkyl)(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-SO—(C₁₋₅ alkyl), —(C₀₋₃ alkylene)-SO₂—(C₁₋₅ alkyl), —(C₀₋₃ alkylene)-carbocyclyl, —(C₀₋₃ alkylene)-heterocyclyl, and -L¹-R^{L1}, wherein the carbocyclyl group in said —(C₀₋₃ alkylene)-carbocyclyl and the heterocyclyl group in said —(C₀₋₃ alkylene)-heterocyclyl are each optionally substituted with one or more (e.g., one, two or three) groups R^{Cyc}.

Preferably, each R^{X31} is independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O—(C₁₋₅ haloalkyl), —CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO—(C₁₋₅ alkyl), —SO₂—(C₁₋₅ alkyl), —(C₀₋₃ alkylene)-carbocyclyl, —(C₀₋₃ alkylene)-heterocyclyl, and -L¹-R^{L1}, wherein the carbocyclyl group in said —(C₀₋₃ alkylene)-carbocyclyl and the heterocyclyl group in said —(C₀₋₃ alkylene)-heterocyclyl are each optionally substituted with one or more groups R^{Cyc}. More preferably, each R^{X31} is independently selected from C₁₋₅ alkyl, C₂₋₅ alkenyl, C₂₋₅ alkynyl, halogen, C₁₋₅ haloalkyl, —O—(C₁₋₅ haloalkyl), —CN, —(C₀₋₃ alkylene)-carbocyclyl, and —(C₀₋₃ alkylene)-heterocyclyl, wherein the carbocyclyl group in said —(C₀₋₃ alkylene)-carbocyclyl and the heterocyclyl group in said —(C₀₋₃ alkylene)-heterocyclyl are each optionally substituted with one or more groups independently selected from C₁₋₅ alkyl, C₂₋₅ alkenyl, C₂₋₅ alkynyl, halogen, C₁₋₅ haloalkyl, —O—(C₁₋₅ haloalkyl), and —CN. Even more preferably, each R^{X31} is independently selected from C₁₋₅ alkyl, halogen, C₁₋₅ haloalkyl, —O—(C₁₋₅ haloalkyl), and —CN. Yet even more preferably, each R^{X31} is independently selected from halogen (e.g., —F, —Cl, —Br, or —I), C₁₋₅ haloalkyl (e.g., —CF₃), and C₁₋₅ alkyl (e.g., methyl). Still more preferably, each R^{X31} is independently halogen (particularly —F or —Cl) or C₁₋₅ haloalkyl (particularly —CF₃).

The group L is selected from —CO—, —SO— and —SO₂—. Preferably, L is —CO—.

Ring A is a 5 to 14 membered heterocyclyl which is attached via a ring nitrogen atom to group L, wherein said heterocyclyl is optionally substituted with one or more (e.g., one, two, three, or four) groups R^{A}.

Preferably, ring A is a 5 to 14 membered heterocycloalkyl or a 5 to 14 membered heterocycloalkenyl, wherein said heterocycloalkyl or said heterocycloalkenyl is attached via a ring nitrogen atom to group L, and wherein said heterocycloalkyl or said heterocycloalkenyl is optionally substituted with one or more groups R^{A}. More preferably, ring A is a 5 to 14 membered heterocycloalkyl which is attached via a ring nitrogen atom to group L, wherein said heterocycloalkyl is optionally substituted with one or more groups R^{A}. Said heterocycloalkyl is preferably a 5 to 11 membered heterocycloalkyl containing one nitrogen ring atom (through which the heterocycloalkyl is attached to group L) and optionally containing one or more (e.g., one, two, or three) further ring heteroatoms independently selected from nitrogen, oxygen and sulfur, wherein all remaining ring atoms are carbon atoms, wherein any nitrogen ring atom (if present) and/or any sulfur ring atom (if present) is optionally oxidized, and wherein one or more carbon ring atoms are optionally oxidized (i.e., to form an oxo group). More preferably, said heterocycloalkyl is a 5 to 7 membered (even more preferably a 6-membered) monocyclic heterocycloalkyl containing one nitrogen ring atom (through which the heterocycloalkyl is attached to group L) and optionally containing one or two further ring heteroatoms independently selected from nitrogen, oxygen and sulfur, wherein all remaining ring atoms are carbon atoms, wherein any nitrogen ring atom (if present) and/or any sulfur ring atom (if present) is optionally oxidized, and wherein one or more carbon ring atoms are optionally oxidized. Moreover, the heterocycloalkyl may contain a lactam function, i.e. the heterocycloalkyl may contain a second nitrogen ring atom (in addition to the first nitrogen ring atom through which ring A is attached to group L) which is adjacent to an oxidized carbon ring atom (C═O). A corresponding preferred example of ring A is 3-oxopiperazin-1-yl which is optionally substituted with one or more (e.g., one, two, three, or four) groups R^{A}. A further preferred example of ring A is 4-(5-carboxypyridin-2-yl)piperazin-1-yl which is optionally substituted with one or more (e.g., one, two, three, or four) groups R^{A}; corresponding preferred examples of ring A include 2,2-dimethyl-4-(5-carboxy-4,6-dimethyl-pyridin-2-yl)piperazin-1-yl or 2,2-dimethyl-4-(5-carboxy-pyridin-2-yl)piperazin-1-yl, particularly 2,2-dimethyl-4-(5-carboxy-4,6-dimethyl-pyridin-2-yl)piperazin-1-yl.

Each R^{A} is independently selected from C₁₋₅ alkyl, C₂₋₅ alkenyl, C₂₋₅ alkynyl, —(CO—₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-NH—OH, —(C₀₋₃ alkylene)-N(C₁₋₅ alkyl)-OH, —(C₀₋₃ alkylene)-NH—O(C₁₋₅ alkyl), —(C₀₋₃ alkylene)-N(C₁₋₅ alkyl)-O(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-NH—COO(C₁₋₅ alkyl), —(C₀₋₃ alkylene)-N(C₁₋₅ alkyl)-COO(C₁₋₅ alkyl), —(C₀₋₃ alkylene)-O—CO—NH(C₁₋₅ alkyl), —(C₀₋₃ alkylene)-O—CO—N(C₁₋₅ alkyl)(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-SO—(C₁₋₅ alkyl), —(C₀₋₃ alkylene)-SO₂—(C₁₋₅ alkyl), —(C₀₋₃ alkylene)-carbocyclyl, —(C₀₋₃ alkylene)-heterocyclyl, and -L¹-R^{L1}, wherein the carbocyclyl group in said —(C₀₋₃ alkylene)-carbocyclyl and the heterocyclyl group in said —(C₀₋₃ alkylene)-heterocyclyl are each optionally substituted with one or more (e.g., one, two or three) groups R^{Cyc}; and further wherein any two groups R^{A}, which are attached to the same carbon ring atom of ring A, may also be mutually joined to form, together with the carbon ring atom that they are attached to, a cycloalkyl or a heterocycloalkyl, wherein said cycloalkyl or said heterocycloalkyl is optionally substituted with one or more (e.g., one, two or three) groups R^{Cyc}.

Preferably, each R^{A} is independently selected from C₁₋₅ alkyl, —(CO—₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-NH—COO(C₁₋₅ alkyl), —(C₀₋₃ alkylene)-N(C₁₋₅ alkyl)-COO(C₁₋₅ alkyl), —(C₀₋₃ alkylene)-O—CO—NH(C₁₋₅ alkyl), —(C₀₋₃ alkylene)-O—CO—N(C₁₋₅ alkyl)(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-aryl, —(C₀₋₃ alkylene)-cycloalkyl (e.g., cyclopropyl), —(C₀₋₃ alkylene)-heteroaryl (e.g., pyridinyl; such as pyridin-2-yl), and —(C₀₋₃ alkylene)-heterocycloalkyl, wherein the aryl group in said —(C₀₋₃ alkylene)-aryl, the cycloalkyl group in said —(C₀₋₃ alkylene)-cycloalkyl, the heteroaryl group in said —(C₀₋₃ alkylene)-heteroaryl, and the heterocycloalkyl group in said —(C₀₋₃ alkylene)-heterocycloalkyl are each optionally substituted with one or more groups R^{Cyc}; and further wherein any two groups R^{A}, which are attached to the same carbon ring atom of ring A, may also be mutually joined to form, together with the carbon ring atom that they are attached to, a cycloalkyl (e.g., a C₃₋₇ cycloalkyl, particularly cyclopropyl).

It is particularly preferred that at least two substituents R^{A} are present, which are attached to the same carbon ring atom of ring A, and which are each independently a C₁₋₅ alkyl group or which are mutually joined to form, together with the carbon ring atom that they are attached to, a C₃₋₇ cycloalkyl group. Accordingly, it is particularly preferred that ring A is a heterocycloalkyl (including any of the specific heterocycloalkyl groups described herein above) which is attached via a ring nitrogen atom to group L, wherein said heterocycloalkyl is either (i) substituted with two C₁₋₅ alkyl groups which are attached to the same ring carbon atom or is (ii) substituted with two substituents R^{A} which are attached to the same ring carbon atom and are mutually joined to form, together with the ring carbon atom that they are attached to, a C₃₋₇ cycloalkyl group (e.g. a cyclopropyl group), and wherein said heterocycloalkyl is optionally further substituted with one or more groups R^{A}. Even more preferably, ring A is a heterocycloalkyl (including any of the specific heterocycloalkyl groups described herein above) which is attached via a ring nitrogen atom to group L, wherein said heterocycloalkyl is substituted with two C₁₋₅ alkyl groups which are attached to the same ring carbon atom, and wherein said heterocycloalkyl is optionally further substituted with one or more groups R^{A} (e.g., with one group R^{A} which is 5-carboxy-4,6-dimethyl-pyridin-2-yl). The two C₁₋₅ alkyl groups that are attached to the same ring carbon atom may be the same or different, and are preferably selected independently from methyl, ethyl, propyl and butyl; more preferably, the two C₁₋₅ alkyl groups that are attached to the same ring carbon atom are each methyl. The C₃₋₇ cycloalkyl group (which is formed from the two mutually joined substituents R^{A}) is preferably selected from cyclopropyl, cyclobutyl, cyclopentyl and cyclohexyl; more preferably, the C₃₋₇ cycloalkyl group is a cyclopropyl group. The position, i.e. the specific carbon ring atom of ring A, at which the two C₁₋₅ alkyl groups or the two mutually joined substituents R^{A} (which together form a C₃₋₇ cycloalkyl group) are attached is not particularly limited. For example, the two C₁₋₅ alkyl groups or the two mutually joined substituents R^{A} (which together form a C₃₋₇ cycloalkyl group) may be attached to a carbon ring atom (of ring A) which is (i) directly adjacent to the nitrogen ring atom through which ring A is attached to group L, or is (ii) separated by one ring atom from said nitrogen ring atom (through which ring A is attached to group L), or is (iii) separated by two ring atoms from said nitrogen ring atom (through which ring A is attached to group L). Corresponding preferred examples of ring A include 2,2-dimethyl-piperazin-1-yl, 3,3-dimethyl-piperazin-1-yl, 2,2-dimethyl-piperazin-3-on-1-yl, spiro[piperazin-2,1′-cyclopropane]-1-yl, spiro[piperazin-3,1′-cyclopropane]-1-yl, 2,2-dimethyl-piperidin-1-yl, 3,3-dimethyl-piperidin-1-yl, 4,4-dimethyl-piperidin-1-yl, spiro[piperidin-2,1′-cyclopropane]-1-yl, spiro[piperidin-3,1′-cyclopropane]-1-yl, or spiro[piperidin-4,1′-cyclopropane]-1-yl, wherein the piperazinyl moiety, the piperazinonyl moiety or the piperidinyl moiety in each of the aforementioned groups is optionally further substituted with one or more (e.g., one or two) groups R^{A} (e.g., with one group R^{A} which is 5-carboxy-4,6-dimethyl-pyridin-2-yl). It is particularly preferred that the two C₁₋₅ alkyl groups or the two mutually joined substituents R^{A} (which together form a C₃₋₇ cycloalkyl group, preferably a cyclopropyl group) are attached to a carbon ring atom which is directly adjacent to the nitrogen ring atom through which ring A is attached to group L. A corresponding particularly preferred example of ring A is 2,2-dimethyl-piperazin-1-yl, wherein the piperazinyl group in said 2,2-dimethyl-piperazin-1-yl is optionally further substituted with one or more groups R^{A}; accordingly, ring A may be, e.g., 2,2-dimethyl-4-(5-carboxy-4,6-dimethyl-pyridin-2-yl)piperazin-1-yl.

In accordance with the above, it is particularly preferred that ring A is selected from any one of the following groups:

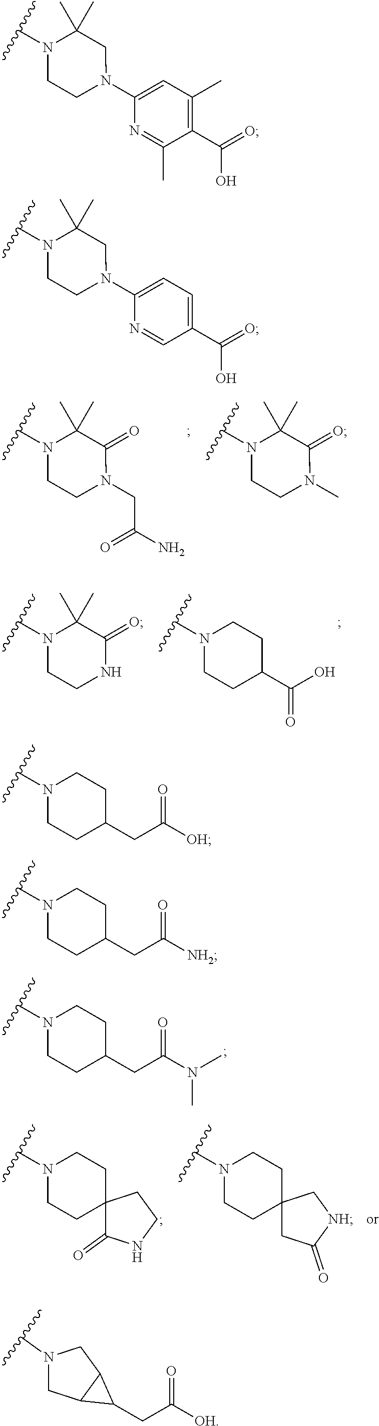

An especially preferred example of ring A is 2,2-dimethyl-4-(5-carboxy-4,6-dimethyl-pyridin-2-yl)piperazin-1-yl:

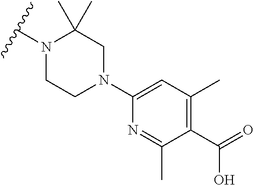 [SMILES: *N1CCN(c2cc(C)c(C(=O)O)c(C)n2)CC1(C)C]

Each R^{Cyc} is independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O—(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), —P(═O)(—OH)(—OH), —P(═O)(—OH)(—O—C₁₋₅ alkyl), —P(═O)(—O—C₁₋₅ alkyl)(-O—C₁₋₅ alkyl), —(C₀₋₃ alkylene)-cycloalkyl, —(C₀₋₃ alkylene)-heterocycloalkyl, and -L¹-R^{L1}.

Preferably, each R^{Cyc} is independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O—(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), —(C₀₋₃ alkylene)-cycloalkyl, —(C₀₋₃ alkylene)-heterocycloalkyl, and -L¹-R^{L1}.

Each L¹ is independently selected from a covalent bond, C₁₋₇ alkylene, C₂₋₇ alkenylene, and C₂₋₇ alkynylene, wherein said alkylene, said alkenylene and said alkynylene are each optionally substituted with one or more (e.g., one, two, or three) groups independently selected from halogen, C₁₋₅ haloalkyl, —O—(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl), and further wherein one or more (e.g., one, two, or three) —CH₂— units comprised in said alkylene, said alkenylene or said alkynylene are each optionally replaced by a group independently selected from —O—, —NH—, —N(C₁₋₅ alkyl)-, —CO—, —S—, —SO—, and —SO₂—.

Preferably, each L¹ is independently selected from a covalent bond, C₁₋₅ alkylene, C₂₋₅ alkenylene, and C₂₋₅ alkynylene, wherein said alkylene, said alkenylene and said alkynylene are each optionally substituted with one or more (e.g., one, two, or three) groups independently selected from halogen, C₁₋₅ haloalkyl, —O—(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl), and further wherein one or more (e.g., one, two, or three) —CH₂— units comprised in said alkylene, said alkenylene or said alkynylene are each optionally replaced by a group independently selected from —O—, —NH—, —N(C₁₋₅ alkyl)-, —CO—, —S—, —SO—, and —SO₂—.

Each R^{L1} is independently selected from —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O—(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C_{1- 5} alkyl), aryl, heteroaryl, cycloalkyl, and heterocycloalkyl, wherein said aryl, said heteroaryl, said cycloalkyl, and said heterocycloalkyl are each optionally substituted with one or more (e.g., one, two or three) groups independently selected from C₁₋₅ alkyl, C₂₋₅ alkenyl, C₂₋₅ alkynyl, halogen, C₁₋₅ haloalkyl, —O—(C₁₋₅ haloalkyl), —CN, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO—(C₁₋₅ alkyl), and —SO₂—(C₁₋₅ alkyl).

Preferably, each R^{L1} is independently selected from —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O—(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C_{1- 5} alkyl), aryl, heteroaryl, cycloalkyl, and heterocycloalkyl, wherein said aryl, said heteroaryl, said cycloalkyl, and said heterocycloalkyl are each optionally substituted with one or more (e.g., one, two or three) groups independently selected from C₁₋₅ alkyl, C₂₋₅ alkenyl, C₂₋₅ alkynyl, halogen, C₁₋₅ haloalkyl, —O—(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl).

In accordance with the present invention, all of the following conditions apply to the compounds of formula (I):
if X¹, X² and X⁷ are nitrogen atoms, X³, X⁴, X⁵, X⁶ and X⁸ are carbon atoms, and L is —CO—, then R^{X3} is aryl, -L^{X3}-cycloalkyl, or monocyclic heteroaryl, wherein said aryl, the cycloalkyl in said -L^{X3}-cycloalkyl, or said heteroaryl is optionally substituted with one or more groups R^{X31};
if X², X³, X⁶ and X⁸ are nitrogen atoms, X¹, X⁴, X⁵ and X⁷ are carbon atoms, L is —CO—, and ring A is a monocyclic heterocyclyl which is attached via a ring nitrogen atom to group L and which is optionally substituted with one or more groups R^{A}, then the carbon atom in position X⁷ is not substituted with C₁₋₅ alkyl, —OH, —O(C₁₋₅ alkyl), —NH₂, —NH(C₁₋₅ alkyl), —N(C₁₋₅ alkyl)(C₁₋₅ alkyl), or phenyl;
if X¹ and X² are nitrogen atoms, X³, X⁴ and X⁵ are carbon atoms, L is —CO—, and R^{X1} is pyrimidin-2-yl which is optionally substituted with one or more groups R^{Cyc}, then R^{X3} is not cycloalkyl;
if X³ is a nitrogen atom, X¹, X⁴ and X⁵ are carbon atoms, and R^{X1} is aryl or heteroaryl wherein said aryl or said heteroaryl is optionally substituted with one or more groups R^{Cyc}, then R^{X3} is not cyclopropyl;
if X³ and X⁸ are nitrogen atoms, X¹, X², X⁴, X⁵, X⁶ and X⁷ are carbon atoms, L is —CO—, and ring A is 2,5-dimethyl-4-(4-fluorobenzyl)-piperazin-1-yl, then R^{X3} is not 4-methoxybenzyl;
if X¹ is a nitrogen atom, X², X³, X⁴, X⁵, X⁶, X⁷ and X⁸ are carbon atoms, and L is —CO—, then ring A is not 4-(C₁₋₅ alkyl)-piperazin-1-yl or 4-(C₃₋₇ cycloalkyl)-piperazin-1-yl;
if X¹ is a nitrogen atom, X³, X⁴, X⁵, X⁶, X⁷ and X⁸ are carbon atoms, and L is —CO—, then R^{X3} is not a 9-membered bicyclic heteroaryl which is optionally substituted with one or more groups R^{X31};
if X¹ is a nitrogen atom, X², X³, X⁴, X⁵, X⁶, X⁷ and X⁸ are carbon atoms, L is —CO—, and R^{X1} is —CH₃ or —SO₂—CH₃, then R^{X3} is not —CH₂-heterocycloalkyl wherein the heterocycloalkyl in said —CH₂-heterocycloalkyl is optionally substituted with one or more groups R^{X31};
if X¹ is a nitrogen atom, X², X³, X⁴, X⁵, X⁶, X⁷ and X⁸ are carbon atoms, L is —CO—, ring A is morpholin-4-yl, and R^{X1} is 4-carboxythiazol-2-yl, then R^{X3} is not 4-sulfamoylbenzyl (i.e., R^{X3} is not 4-(H₂N—SO₂)-phenylmethyl);
if X³ is a nitrogen atom, X¹, X², X⁴, X⁵, X⁶, X⁷ and X⁸ are carbon atoms, L is —CO—, ring A is morpholin-4-yl, and R^{X1} is 4-sulfamoylbenzyl (i.e., 4-(H₂N—SO₂)-phenylmethyl), then R^{X3} is not 4-carboxythiazol-2-yl;
if X³ is a nitrogen atom, X¹, X², X⁴, X⁵, X⁶, X⁷ and X⁸ are carbon atoms, and L is —CO—, then L^{X3} is not —C(R^{LX3})(R^{LX3})—;
if X² and X³ are nitrogen atoms, X¹, X⁴, X⁵, X⁶, X⁷ and X⁸ are carbon atoms, and L is —SO₂—, then L^{X3} is not —CH₂—;
if X², X³ and X⁸ are nitrogen atoms, X¹, X⁴, X⁵, X⁶ and X⁷ are carbon atoms, L is —CO—, R^{X1} is methyl, and R^{X3} is pyridin-2-yl, then ring A is not piperidin-1-yl.

Moreover, it is preferred that the following condition also applies to the compounds of formula (I): if X³ is a nitrogen atom, X¹, X⁴ and X⁵ are carbon atoms, and R^{X1} is phenyl, pyridin-2-yl or pyrimidin-2-yl wherein said phenyl, said pyridin-2-yl or said pyrimidin-2-yl is optionally substituted with one or more groups R^{Cyc}, then R^{X3} is not cycloalkyl. More preferably, if X¹ or X³ is a nitrogen atom, X⁴ and X⁵ are carbon atoms, and R^{X1} is phenyl, pyridin-2-yl or pyrimidin-2-yl wherein said phenyl, said pyridin-2-yl or said pyrimidin-2-yl is optionally substituted with one or more groups R^{Cyc}, then R^{X3} is not cycloalkyl.

It is furthermore preferred that the following condition also applies to the compounds of formula (I): if X², X³, X⁶ and X⁸ are nitrogen atoms, X¹, X⁴, X⁵ and X⁷ are carbon atoms, L is —CO—, and ring A is a monocyclic heterocyclyl which is attached via a ring nitrogen atom to group L and which is optionally substituted with one or more groups R^{A}, then the carbon atom in position X⁷ is not substituted with any group R^{X} (i.e., X⁷ is a carbon atom which is unsubstituted).

Preferably, the following condition also applies to the compounds of formula (I): if X¹ is a nitrogen atom, X², X³, X⁴, X⁵, X⁶, X⁷ and X⁸ are carbon atoms, and L is —CO—, then R^{X3} is not pyrrolidinyl, piperidinyl, 1,2,3,6-tetrahydropyridinyl or morpholinyl, wherein said pyrrolidinyl, said piperidinyl, said 1,2,3,6-tetrahydropyridinyl or said morpholinyl is optionally substituted with one or more groups R^{X31}.

Preferably, the following condition also applies to the compounds of formula (I): if X¹ is a nitrogen atom, X², X³, X⁴, X⁵, X⁶, X⁷ and X⁸ are carbon atoms, L is —CO—, and R^{X1} is —CH₃ or —SO₂—CH₃, then L^{X3} is not —CH₂—. More preferably, if X¹ is a nitrogen atom, X², X³, X⁴, X⁵, X⁶, X⁷ and X⁸ are carbon atoms, L is —CO—, and R^{X1} is —CH₃ or —SO₂—CH₃, then L^{X3} is not —C(R^{LX3})(R^{LX3})—.

It is preferred that the following conditions also apply to the compounds of formula (I):
if X¹ is a nitrogen atom, X², X³, X⁴, X⁵, X⁷ and X⁸ are carbon atoms, and R^{X1} is a 5-membered heteroaryl wherein said 5-membered heteroaryl is optionally substituted with one or more groups R^{Cyc}, then R^{X3} is not -L^{X3}-phenyl wherein the phenyl in said -L^{X3}-phenyl is optionally substituted with one or more groups R^{X31};
if X³ is a nitrogen atom, X¹, X², X⁴, X⁵, X⁶ and X⁷ are carbon atoms, and R^{X3} is a 5-membered heteroaryl wherein said 5-membered heteroaryl is optionally substituted with one or more groups R^{X31}, then R^{X1} is not —(C₀₋₅ alkylene)-phenyl, wherein the alkylene group in said —(C₀₋₅ alkylene)-phenyl is optionally substituted with one or more groups R^{X11}, wherein one or more —CH₂— units comprised in in the alkylene group in said —(C₀₋₅ alkylene)-phenyl are each optionally replaced by a group independently selected from —O—, —NH—, —N(C₁₋₅ alkyl)-, —CO—, —S—, —SO—, and —SO₂—, and wherein the phenyl group in said —(C₀₋₅ alkylene)-phenyl is optionally substituted with one or more groups R^{Cyc}.

Preferably, the following condition also applies to the compounds of formula (I): if X², X³ and X⁸ are nitrogen atoms, and X¹, X⁴, X⁵, X⁶ and X⁷ are carbon atoms, then R^{X3} is -L^{X3}-carbocyclyl, wherein the carbocyclyl in said -L^{X3}-carbocyclyl is optionally substituted with one or more groups R^{X31}.

Preferably, the following condition also applies to the compounds of formula (I): if X² and X³ are nitrogen atoms, and X¹, X⁴, X⁵, X⁶, X⁷ and X⁸ are carbon atoms, then L is not —SO₂—.

Preferably, the following condition also applies to the compounds of formula (I): if L is —SO₂—, then L^{X3} is not —CH₂—.

Preferably, the following condition also applies to the compounds of formula (I): if X³ is a nitrogen atom, X¹, X², X⁴, X⁵, X⁶, X⁷ and X⁸ are carbon atoms, and L is —CO—, then R^{X1} is not methyl.

It is particularly preferred that the compound of formula (I) is any one of the specific compounds of formula (I) described in the examples section of this specification, including any one of Examples 1 to 226 described further below, either in non-salt form and/or non-solvated form, or as a pharmaceutically acceptable salt or solvate of the respective compound.

Accordingly, it is particularly preferred that the compound of formula (I) is selected from:
4-(1-(tert-butyl)-3-(4-chloro-3-fluorophenyl)-1H-pyrrolo[2,3-b]pyridine-6-carbonyl)-3,3-dimethylpiperazin-2-one;
4-(1-(tert-butyl)-3-(4-chloro-3-fluorophenyl)-1H-pyrrolo[2,3-b]pyridine-6-carbonyl)-1,3,3-trimethylpiperazin-2-one;
methyl 2-(4-(1-(tert-butyl)-3-(4-chloro-3-fluorophenyl)-1H-pyrrolo[2,3-b]pyridine-6-carbonyl)-3,3-dimethyl-2-oxopiperazin-1-yl)acetate;
2-(4-(1-(tert-butyl)-3-(4-chloro-3-fluorophenyl)-1H-pyrrolo[2,3-b]pyridine-6-carbonyl)-3,3-dimethyl-2-oxopiperazin-1-yl)acetic acid;
2-(4-(1-(tert-butyl)-3-(4-chloro-3-fluorophenyl)-1H-pyrrolo[2,3-b]pyridine-6-carbonyl)-3,3-dimethyl-2-oxopiperazin-1-yl)acetamide;
2-(4-(1-(tert-butyl)-3-(4-chloro-3-fluorophenyl)-1H-pyrrolo[2,3-b]pyridine-6-carbonyl)-3,3-dimethyl-2-oxopiperazin-1-yl)-N-methylacetamide;
2-(4-(1-(tert-butyl)-3-(4-chloro-3-fluorophenyl)-1H-pyrrolo[2,3-b]pyridine-6-carbonyl)-3,3-dimethyl-2-oxopiperazin-1-yl)-N,N-dimethylacetamide;
4-(1-(tert-butyl)-3-(4-chloro-3-fluorophenyl)-1H-pyrrolo[2,3-b]pyridine-6-carbonyl)-3,3-dimethyl-1-(2-morpholino-2-oxoethyl)piperazin-2-one;
(1-(tert-butyl)-3-(4-chloro-3-fluorophenyl)-1H-pyrrolo[2,3-b]pyridin-6-yl)(6-oxa-2-azaspiro[3.4]octan-2-yl)methanone; (1-(tert-butyl)-3-(4-chloro-3-fluorophenyl)-1H-pyrrolo[2,3-b]pyridin-6-yl)(2-oxa-6-azaspiro[3.3]heptan-6-yl)methanone;
(1-(tert-butyl)-3-(4-chloro-3-fluorophenyl)-1H-pyrrolo[2,3-b]pyridin-6-yl)((1R,3r,5S)-3-hydroxy-8-azabicyclo[3.2.1]octan-8-yl)methanone;
(1-(tert-butyl)-3-(4-chloro-3-fluorophenyl)-1H-pyrrolo[2,3-b]pyridin-6-yl)((1R,3s,5S)-3-hydroxy-8-azabicyclo[3.2.1]octan-8-yl)methanone;
8-(1-(tert-butyl)-3-(4-chloro-3-fluorophenyl)-1H-pyrrolo[2,3-b]pyridine-6-carbonyl)-2,8-diazaspiro[4.5]decan-1-one;
8-(1-(tert-butyl)-3-(4-chloro-3-fluorophenyl)-1H-pyrrolo[2,3-b]pyridine-6-carbonyl)-2,8-diazaspiro[4.5]decan-3-one;
(3aS,6aS)-1-(1-(tert-butyl)-3-(4-chloro-3-fluorophenyl)-1H-pyrrolo[2,3-b]pyridine-6-carbonyl)hexahydropyrrolo[3,4-b]pyrrol-6(1H)-one;
(1-(tert-butyl)-3-(4-chloro-3-fluorophenyl)-1H-pyrrolo[2,3-b]pyridin-6-yl)(2,2-dimethylpyrrolidin-1-yl)methanone;
4-(1-(tert-butyl)-3-(4-chloro-3-fluorophenyl)-1H-pyrrolo[2,3-b]pyridine-6-carbonyl)-1,4-diazepan-2-one;
1-(1-(tertbutyl)-3-(4-chloro-3-fluorophenyl)-1H-pyrrolo[2,3-b]pyridine-6-carbonyl)-1,4-diazepan-5-one;
((1R,5S)-3-oxa-8-azabicyclo[3.2.1]octan-8-yl)(1-(tertbutyl)-3-(4-chloro-3-fluorophenyl)-1H-pyrrolo[2,3-b]pyridin-6-yl)methanone;
4-(1-(tertbutyl)-3-(4-chloro-3-fluorophenyl)-1H-pyrrolo[2,3-b]pyridine-6-carbonyl)-1-methylpiperazin-2-one;
methyl 1-(1-(tert-butyl)-3-(4-chloro-3-fluorophenyl)-1H-pyrrolo[2,3-b]pyridine-6-carbonyl)-3,3-dimethylpiperidine-4-carboxylate;
1-(1-(tert-butyl)-3-(4-chloro-3-fluorophenyl)-1H-pyrrolo[2,3-b]pyridine-6-carbonyl)-3,3-dimethylpiperidine-4-carboxylic acid;
1-(1-(tert-butyl)-3-(4-chloro-3-fluorophenyl)-1H-pyrrolo[2,3-b]pyridine-6-carbonyl)-3,3-dimethylpiperidine-4-carboxamide;
8-(1-(tert-butyl)-3-(4-chloro-3-fluorophenyl)-1H-pyrrolo[2,3-b]pyridine-6-carbonyl)-1,3,8-triazaspiro[4.5]decane-2,4-dione;
methyl 6-(4-(1-(tert-butyl)-3-(4-chloro-3-fluorophenyl)-1H-pyrrolo[2,3-b]pyridine-6-carbonyl)-3,3-dimethylpiperazin-1-yl)-2,4-dimethylnicotinate;
6-(4-(1-(tert-butyl)-3-(4-chloro-3-fluorophenyl)-1H-pyrrolo[2,3-b]pyridine-6-carbonyl)-3,3-dimethylpiperazin-1-yl)-2,4-dimethylnicotinic acid;
4-(3-(4-chloro-3-fluorophenyl)-1-(4-methoxybenzyl)-1H-pyrrolo[2,3-b]pyridine-6-carbonyl)-3,3-dimethylpiperazin-2-one;
4-(3-(4-chloro-3-fluorophenyl)-1-(3-methoxycyclobutyl)-1H-pyrrolo[2,3-b]pyridine-6-carbonyl)-3,3-dimethylpiperazin-2-one;
4-(3-(4-chloro-3-fluorophenyl)-1-isobutyl-1H-pyrrolo[2,3-b]pyridine-6-carbonyl)-3,3-dimethylpiperazin-2-one;
4-(3-(4-chloro-3-fluorophenyl)-1-(tetrahydro-2H-pyran-4-yl)-1H-pyrrolo[2,3-b]pyridine-6-carbonyl)-3,3-dimethylpiperazin-2-one;
4-(3-(4-chloro-3-fluorophenyl)-1-(2,2,2-trifluoroethyl)-1H-pyrrolo[2,3-b]pyridine-6-carbonyl)-3,3-dimethylpiperazin-2-one;
(3-(4-chloro-3-fluorophenyl)-1-(2,2,2-trifluoroethyl)-1H-pyrrolo[2,3-b]pyridin-6-yl)(1,4-oxazepan-4-yl)methanone;
(8-oxa-3-azabicyclo[3.2.1]octan-3-yl)(3-(4-chloro-3-fluorophenyl)-1-(2,2,2-trifluoroethyl)-1H-pyrrolo[2,3-b]pyridin-6-yl)methanone;
4-(3-(4-chloro-3-fluorophenyl)-1-methyl-1H-pyrrolo[2,3-b]pyridine-6-carbonyl)-3,3-dimethylpiperazin-2-one;
4-(3-(4-chloro-3-fluorophenyl)-1-(cyclopropylmethyl)-1H-pyrrolo[2,3-b]pyridine-6-carbonyl)-3,3-dimethylpiperazin-2-one;
4-(1-isobutyl-3-(pyrimidin-5-yl)-1H-pyrrolo[2,3-b]pyridine-6-carbonyl)-3,3-dimethylpiperazin-2-one;
4-(1-isobutyl-3-(pyridin-4-yl)-1H-pyrrolo[2,3-b]pyridine-6-carbonyl)-3,3-dimethylpiperazin-2-one;
4-(3-(4-chloro-3-fluorophenyl)-1-isobutyl-2-methyl-1H-pyrrolo[2,3-b]pyridine-6-carbonyl)-3,3-dimethylpiperazin-2-one;
4-(3-(4-chloro-3-fluorophenyl)-1-(2-(2,2,2-trifluoroethoxy)ethyl)-1H-pyrrolo[2,3-b]pyridine-6-carbonyl)-3,3-dimethylpiperazin-2-one;
4-(1-(2-(1H-1,2,4-triazol-1-yl)ethyl)-3-(4-chloro-3-fluorophenyl)-1H-pyrrolo[2,3-b]pyridine-6-carbonyl)-3,3-dimethylpiperazin-2-one;
4-(3-(4-chloro-3-fluorophenyl)-1-(2-(2-oxopyrrolidin-1-yl)ethyl)-1H-pyrrolo[2,3-b]pyridine-6-carbonyl)-3,3-dimethylpiperazin-2-one;
4-(3-(4-chloro-3-fluorophenyl)-1-(2-ethoxyethyl)-1H-pyrrolo[2,3-b]pyridine-6-carbonyl)-3,3-dimethylpiperazin-2-one;
4-(1-isobutyl-3-(6-(trifluoromethyl)pyridin-3-yl)-1H-pyrrolo[2,3-b]pyridine-6-carbonyl)-3,3-dimethylpiperazin-2-one;
4-(1-isobutyl-3-(1-methyl-1H-pyrazol-4-yl)-1H-pyrrolo[2,3-b]pyridine-6-carbonyl)-3,3-dimethylpiperazin-2-one;
4-(3-(3,6-dihydro-2H-pyran-4-yl)-1-isobutyl-1H-pyrrolo[2,3-b]pyridine-6-carbonyl)-3,3-dimethylpiperazin-2-one;
4-(1-isobutyl-3-(tetrahydro-2H-pyran-4-yl)-1H-pyrrolo[2,3-b]pyridine-6-carbonyl)-3,3-dimethylpiperazin-2-one;
4-(3-(1,2,3,6-tetrahydropyridin-4-yl)-1-isobutyl-1H-pyrrolo[2,3-b]pyridine-6-carbonyl)-3,3-dimethylpiperazin-2-one;
4-(3-cyclohexyl-1-isobutyl-1H-pyrrolo[2,3-b]pyridine-6-carbonyl)-3,3-dimethylpiperazin-2-one;
4-(1-isobutyl-3-phenyl-pyrrolo[2,3-b]pyridine-6-carbonyl)-3,3-dimethyl-piperazin-2-one;
4-[3-(3-chlorophenyl)-1-isobutyl-pyrrolo[2,3-b]pyridine-6-carbonyl]-3,3-dimethyl-piperazin-2-one;
4-[1-isobutyl-3-(p-tolyl)pyrrolo[2,3-b]pyridine-6-carbonyl]-3,3-dimethyl-piperazin-2-one;
4-[1-isobutyl-3-(o-tolyl)pyrrolo[2,3-b]pyridine-6-carbonyl]-3,3-dimethyl-piperazin-2-one;
4-[3-(2-chlorophenyl)-1-isobutyl-pyrrolo[2,3-b]pyridine-6-carbonyl]-3,3-dimethyl-piperazin-2-one;
4-[3-(4-chlorophenyl)-1-isobutyl-pyrrolo[2,3-b]pyridine-6-carbonyl]-3,3-dimethyl-piperazin-2-one;
4-[1-isobutyl-3-[4-(trifluoromethyl)phenyl]pyrrolo[2,3-b]pyridine-6-carbonyl]-3,3-dimethyl-piperazin-2-one;
4-[1-isobutyl-3-[4-fluorophenyl]pyrrolo[2,3-b]pyridine-6-carbonyl]-3,3-dimethyl-piperazin-2-one;
4-[1-isobutyl-3-[3-(trifluoromethoxy)phenyl]pyrrolo[2,3-b]pyridine-6-carbonyl]-3,3-dimethyl-piperazin-2-one;
methyl 6-(4-(3-(tert-butyl)-1-(4-chloro-3-fluorophenyl)-1H-pyrazolo[4,3-b]pyridine-5-carbonyl)-3,3-dimethylpiperazin-1-yl)-2,4-dimethylnicotinate;
6-(4-(3-(tert-butyl)-1-(4-chloro-3-fluorophenyl)-1H-pyrazolo[4,3-b]pyridine-5-carbonyl)-3,3-dimethylpiperazin-1-yl)-2,4-dimethylnicotinic acid;
6-(4-(3-(tert-butyl)-1-(4-chloro-3-methoxyphenyl)-1H-pyrazolo[4,3-b]pyridine-5-carbonyl)-3,3-dimethylpiperazin-1-yl)-2,4-dimethylnicotinic acid;
4-(1-(2-(1H-pyrazol-1-yl)ethyl)-3-(4-chloro-3-fluorophenyl)-1H-pyrrolo[2,3-b]pyridine-6-carbonyl)-3,3-dimethylpiperazin-2-one;
4-(1-(4-chloro-3-fluorophenyl)-3-isobutyl-1H-pyrazolo[4,3-b]pyridine-5-carbonyl)-3,3-dimethylpiperazin-2-one;
4-(1-(4-chloro-3-fluorophenyl)-3-isobutyl-1H-pyrazolo[4,3-b]pyridine-5-carbonyl)-1-methylpiperazin-2-one;
methyl 6-(4-(1-(4-chloro-3-fluorophenyl)-3-isobutyl-1H-pyrazolo[4,3-b]pyridine-5-carbonyl)-3,3-dimethylpiperazin-1-yl)-2,4-dimethylnicotinate;
6-(4-(1-(4-chloro-3-fluorophenyl)-3-isobutyl-1H-pyrazolo[4,3-b]pyridine-5-carbonyl)-3,3-dimethylpiperazin-1-yl)-2,4-dimethylnicotinic acid;
4-(3-isobutyl-1-(6-(trifluoromethyl)pyridin-3-yl)-1H-pyrazolo[4,3-b]pyridine-5-carbonyl)-3,3-dimethylpiperazin-2-one;
4-(1-(3-chlorophenyl)-3-isobutyl-1H-pyrazolo[4,3-b]pyridine-5-carbonyl)-3,3-dimethylpiperazin-2-one;
methyl 1-(1-(4-chloro-3-fluorophenyl)-3-isobutyl-1H-pyrazolo[4,3-b]pyridine-5-carbonyl)-3,3-dimethylpiperidine-4-carboxylate;
1-(1-(4-chloro-3-fluorophenyl)-3-isobutyl-1H-pyrazolo[4,3-b]pyridine-5-carbonyl)-3,3-dimethylpiperidine-4-carboxylic acid;
8-(1-(4-chloro-3-fluorophenyl)-3-isobutyl-1H-pyrazolo[4,3-b]pyridine-5-carbonyl)-1,3,8-triazaspiro[4.5]decane-2,4-dione;
4-(1-(4-chloro-3-fluorophenyl)-3-methyl-1H-pyrazolo[4,3-b]pyridine-5-carbonyl)-3,3-dimethylpiperazin-2-one;
4-(1-(4-chloro-3-fluorophenyl)-3-(tetrahydro-2H-pyran-4-yl)-1H-pyrazolo[4,3-b]pyridine-5-carbonyl)-3,3-dimethylpiperazin-2-one;
4-(1-(2,4-dichlorophenyl)-3-isobutyl-1H-pyrazolo[4,3-b]pyridine-5-carbonyl)-3,3-dimethylpiperazin-2-one;
4-(1-(2-chlorophenyl)-3-isobutyl-1H-pyrazolo[4,3-b]pyridine-5-carbonyl)-3,3-dimethylpiperazin-2-one;
3-(tert-butyl)-1-(4-chloro-3-fluorophenyl)-1H-pyrazolo[4,3-b]pyridine-5-carbonyl)-3,3-dimethylpiperazin-2-one;
4-(1-(4-chloro-3-fluorophenyl)-3-cyclopentyl-1H-pyrazolo[4,3-b]pyridine-5-carbonyl)-3,3-dimethylpiperazin-2-one;
4-(1-(4-chloro-3-fluorophenyl)-3-cyclopropyl-1H-pyrazolo[4,3-b]pyridine-5-carbonyl)-3,3-dimethylpiperazin-2-one;
4-(1-(4-chloro-3-fluorobenzyl)-3-isobutyl-1H-pyrazolo[4,3-b]pyridine-5-carbonyl)-3,3-dimethylpiperazin-2-one;
4-(3-isobutyl-1-(4-(trifluoromethoxy)phenyl)-1H-pyrazolo[4,3-b]pyridine-5-carbonyl)-3,3-dimethylpiperazin-2-one;
4-(3-(tert-butyl)-1-(4-chloro-3-fluorophenyl)-1H-pyrazolo[3,4-b]pyridine-5-carbonyl)-3,3-dimethylpiperazin-2-one;
1-(4-chloro-3-fluorophenyl)-3-isobutyl-6-methyl-1H-pyrazolo[4,3-b]pyridine-5-carbonyl)-3,3-dimethylpiperazin-2-one;
4-(1-isobutyl-3-(piperidin-1-yl)-1H-pyrrolo[2,3-b]pyridine-6-carbonyl)-3,3-dimethylpiperazin-2-one;
4-(3-(4-chloro-3-fluorophenyl)-1-isobutyl-1H-indazole-6-carbonyl)-3,3-dimethylpiperazin-2-one;
4-(3-(2,4-dichlorophenyl)-1-isobutyl-1H-pyrrolo[2,3-b]pyridine-6-carbonyl)-3,3-dimethylpiperazin-2-one;
4-(1-(4-chlorophenyl)-3-isobutyl-1H-pyrazolo[4,3-b]pyridine-5-carbonyl)-3,3-dimethylpiperazin-2-one;
methyl 6-(4-(1-(tert-butyl)-3-(4-chloro-3-fluorophenyl)-1H-pyrazolo[3,4-b]pyridine-6-carbonyl)-3,3-dimethylpiperazin-1-yl)-2,4-dimethylnicotinate;
6-(4-(1-(tert-butyl)-3-(4-chloro-3-fluorophenyl)-1H-pyrazolo[3,4-b]pyridine-6-carbonyl)-3,3-dimethylpiperazin-1-yl)-2,4-dimethylnicotinic acid;
4-(1-(4-chloro-3-fluorophenyl)-3-isobutyl-1H-pyrazolo[4,3-b]pyridine-5-carbonyl)piperazin-2-one;
(3-(4-chloro-3-fluorophenyl)-1-(2,2,2-trifluoroethyl)-1H-pyrazolo[4,3-c]pyridin-6-yl)(1,4-oxazepan-4-yl)methanone;
(8-oxa-3-azabicyclo[3.2.1]octan-3-yl)(3-(4-chloro-3-fluorophenyl)-1-(2,2,2-trifluoroethyl)-1H-pyrazolo[4,3-c]pyridin-6-yl)methanone;
4-(1-isobutyl-3-(1-methyl-1,2,3,6-tetrahydropyridin-4-yl)-1H-pyrrolo[2,3-b]pyridine-6-carbonyl)-3,3-dimethylpiperazin-2-one;
4-(3-isobutyl-1-(6-methoxypyridin-3-yl)-1H-pyrazolo[4,3-b]pyridine-5-carbonyl)-3,3-dimethylpiperazin-2-one;
4-(3-isobutyl-1-(4-methoxyphenyl)-1H-pyrazolo[4,3-b]pyridine-5-carbonyl)-3,3-dimethylpiperazin-2-one;
8-(1-(4-chloro-3-fluorophenyl)-3-isobutyl-1H-pyrazolo[4,3-b]pyridine-5-carbonyl)-1-phenyl-1,3,8-triazaspiro[4.5]decan-4-one;
6-(4-(3-(4-chloro-3-fluorophenyl)-1-isobutyl-1H-pyrrolo[2,3-b]pyridine-6-carbonyl)-3,3-dimethylpiperazin-1-yl)-2,4-dimethylnicotinic acid;
4-(3-(4-chloro-3-fluorophenyl)-1-isopropyl-1H-pyrrolo[2,3-b]pyridine-6-carbonyl)-3,3-dimethylpiperazin-2-one;
6-(4-(3-(4-chloro-3-fluorophenyl)-1-isopropyl-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-(tetrahydro-2H-pyran-4-yl)-1H-pyrrolo[2,3-b]pyridine-6-carbonyl)-3,3-dimethylpiperazin-1-yl)-2,4-dimethylnicotinic acid;
6-(4-(1-(tert-butyl)-3-(4-chloro-3-fluorophenyl)-1H-pyrrolo[2,3-b]pyridine-6-carbonyl)piperazin-1-yl)-2,4-dimethylnicotinic acid;
6-((1-(1-(tert-butyl)-3-(4-chloro-3-fluorophenyl)-1H-pyrrolo[2,3-b]pyridine-6-carbonyl)piperidin-4-yl)amino)-2,4-dimethylnicotinic acid;
6-(6-(1-(tert-butyl)-3-(4-chloro-3-fluorophenyl)-1H-pyrrolo[2,3-b]pyridine-6-carbonyl)-3,6-diazabicyclo[3.2.0]heptan-3-yl)-2,4-dimethylnicotinic acid;
6-(4-(3-(4-chloro-3-fluorophenyl)-1-(1-hydroxy-2-methylpropan-2-yl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-(pyrazin-2-ylmethyl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-isobutyl-2-methyl-1H-pyrrolo[2,3-b]pyridine-6-carbonyl)-3,3-dimethylpiperazin-1-yl)-2,4-dimethylnicotinic acid;
4-(1-isobutyl-3-(6-methoxypyridin-3-yl)-1H-pyrrolo[2,3-b]pyridine-6-carbonyl)-3,3-dimethylpiperazin-2-one;
4-(6-(2,2-dimethyl-3-oxopiperazine-1-carbonyl)-1-isobutyl-1H-pyrrolo[2,3-b]pyridin-3-yl)benzonitrile; 4-(3-(3,4-dichlorophenyl)-1-isobutyl-1H-pyrrolo[2,3-b]pyridine-6-carbonyl)-3,3-dimethylpiperazin-2-one;
4-(3-(3,5-difluorophenyl)-1-isobutyl-1H-pyrrolo[2,3-b]pyridine-6-carbonyl)-3,3-dimethylpiperazin-2-one;
4-(3-(6-(dimethylamino)pyridin-3-yl)-1-isobutyl-1H-pyrrolo[2,3-b]pyridine-6-carbonyl)-3,3-dimethylpiperazin-2-one;
5-(6-(2,2-dimethyl-3-oxopiperazine-1-carbonyl)-1-isobutyl-1H-pyrrolo[2,3-b]pyridin-3-yl)picolinonitrile; 4-(1-isobutyl-3-(4-methoxyphenyl)-1H-pyrrolo[2,3-b]pyridine-6-carbonyl)-3,3-dimethylpiperazin-2-one;
4-(1-isobutyl-3-(2-(trifluoromethoxy)phenyl)-1H-pyrrolo[2,3-b]pyridine-6-carbonyl)-3,3-dimethylpiperazin-2-one;
4-(3-(2-fluorophenyl)-1-isobutyl-1H-pyrrolo[2,3-b]pyridine-6-carbonyl)-3,3-dimethylpiperazin-2-one;
4-(3-(3-fluorophenyl)-1-isobutyl-1H-pyrrolo[2,3-b]pyridine-6-carbonyl)-3,3-dimethylpiperazin-2-one;
4-(3-(4-chloro-2-fluorophenyl)-1-isobutyl-1H-pyrrolo[2,3-b]pyridine-6-carbonyl)-3,3-dimethylpiperazin-2-one;
4-(1-isobutyl-3-(4-(trifluoromethoxy)phenyl)-1H-pyrrolo[2,3-b]pyridine-6-carbonyl)-3,3-dimethylpiperazin-2-one;
3-(6-(2,2-dimethyl-3-oxopiperazine-1-carbonyl)-1-isobutyl-1H-pyrrolo[2,3-b]pyridin-3-yl)benzonitrile;
4-(1-isobutyl-3-(m-tolyl)-1H-pyrrolo[2,3-b]pyridine-6-carbonyl)-3,3-dimethylpiperazin-2-one;
6-(4-(3-(4-chloro-3-fluorophenyl)-1,2-dimethyl-1H-pyrrolo[2,3-b]pyridine-6-carbonyl)-3,3-dimethylpiperazin-1-yl)-2,4-dimethylnicotinic acid;
4-(3-(4-chloro-3-fluorophenyl)-1-(pyridin-4-ylmethyl)-1H-pyrrolo[2,3-b]pyridine-6-carbonyl)-3,3-dimethylpiperazin-2-one;
6-(4-(3-(4-chloro-3-fluorophenyl)-1-(1-methylcyclobutyl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-methyl-1H-pyrrolo[2,3-b]pyridine-6-carbonyl)-3,3-dimethylpiperazin-1-yl)-2,4-dimethylnicotinic acid;
6-(1-(1-(tert-butyl)-3-(4-chloro-3-fluorophenyl)-1H-pyrrolo[2,3-b]pyridine-6-carbonyl)piperidine-4-carboxamido)-2,4-dimethylnicotinic acid;
2-((1R,5S,6s)-3-(1-(tert-butyl)-3-(4-chloro-3-fluorophenyl)-1H-pyrrolo[2,3-b]pyridine-6-carbonyl)-3-azabicyclo[3.1.0]hexan-6-yl)acetic acid;
2-(1-(1-(tert-butyl)-3-(4-chloro-3-fluorophenyl)-1H-pyrrolo[2,3-b]pyridine-6-carbonyl)piperidin-4-yl)acetic acid;
6-(4-(3-(4-chloro-3-fluorophenyl)-1-(1-ethoxy-2-methylpropan-2-yl)-1H-pyrrolo[2,3-b]pyridine-6-carbonyl)-3,3-dimethylpiperazin-1-yl)-2,4-dimethylnicotinic acid;
6-(4-(1-(tert-butyl)-3-(4-chloro-3-fluorophenyl)-1H-pyrrolo[2,3-b]pyridine-6-carbonyl)-1,4-diazepan-1-yl)-2,4-dimethylnicotinic acid;
6-(7-(1-(tert-butyl)-3-(4-chloro-3-fluorophenyl)-1H-pyrrolo[2,3-b]pyridine-6-carbonyl)-2,7-diazaspiro[4.4]nonan-2-yl)-2,4-dimethylnicotinic acid;
6-(4-(3-(4-chloro-3-fluorophenyl)-1-ethyl-1H-pyrrolo[2,3-b]pyridine-6-carbonyl)-3,3-dimethylpiperazin-1-yl)-2,4-dimethylnicotinic acid;
(R)-6-(4-(1-(tert-butyl)-3-(4-chloro-3-fluorophenyl)-1H-pyrrolo[2,3-b]pyridine-6-carbonyl)-3-methylpiperazin-1-yl)-2,4-dimethylnicotinic acid;
6-(4-(3-(4-chloro-3-fluorophenyl)-1-(2-(pyridin-2-yl)propan-2-yl)-1H-pyrrolo[2,3-b]pyridine-6-carbonyl)-3,3-dimethylpiperazin-1-yl)-2,4-dimethylnicotinic acid;
6-(4-(1-(tert-butyl)-3-(4-chloro-3-fluorophenyl)-1H-pyrrolo[2,3-b]pyridine-6-carbonyl)-2-(methoxymethyl)piperazin-1-yl)-2,4-dimethylnicotinic acid;
6-(4-(1-(carboxymethyl)-3-(4-chloro-3-fluorophenyl)-1H-pyrrolo[2,3-b]pyridine-6-carbonyl)-3,3-dimethylpiperazin-1-yl)-2,4-dimethylnicotinic acid;
6-(4-(3-(3-chloro-4-fluorophenyl)-1-(2-methoxyethyl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-(pyridin-3-ylmethyl)-1H-pyrrolo[2,3-b]pyridine-6-carbonyl)-3,3-dimethylpiperazin-1-yl)-2,4-dimethylnicotinic acid;
(S)-6-(4-(3-(4-chloro-3-fluorophenyl)-1-(1-methoxypropan-2-yl)-1H-pyrrolo[2,3-b]pyridine-6-carbonyl)-3,3-dimethylpiperazin-1-yl)-2,4-dimethylnicotinic acid;
6-(4-(1-(tert-butyl)-3-(4-chloro-3-fluorophenyl)-1H-pyrrolo[2,3-b]pyridine-6-carbonyl)-3,3-dimethylpiperazin-1-yl)nicotinic acid;
6-(4-(1-(tert-butyl)-3-(4-chloro-3-fluorophenyl)-1H-pyrrolo[2,3-b]pyridine-6-carbonyl)-3,3-dimethylpiperazin-1-yl)nicotinamide;
6-(4-(1-(tert-butyl)-3-(4-chloro-3-fluorophenyl)-1H-pyrrolo[2,3-b]pyridine-6-carbonyl)-3,3-dimethylpiperazin-1-yl)-N,N-dimethylnicotinamide;
(S)-6-(4-(3-(4-chloro-3-fluorophenyl)-1-(1-methoxypropan-2-yl)-1H-pyrrolo[2,3-b]pyridine-6-carbonyl)-3,3-dimethylpiperazin-1-yl)-2,4-dimethylnicotinic acid;
6-(4-(3-(3,4-difluorophenyl)-1-isobutyl-1H-pyrrolo[2,3-b]pyridine-6-carbonyl)-3,3-dimethylpiperazin-1-yl)-2,4-dimethylnicotinic acid;
6-(4-(3-(3-fluoro-4-methylphenyl)-1-isobutyl-1H-pyrrolo[2,3-b]pyridine-6-carbonyl)-3,3-dimethylpiperazin-1-yl)-2,4-dimethylnicotinic acid;
6-(4-(1-isobutyl-3-(3,4,5-trifluorophenyl)-1H-pyrrolo[2,3-b]pyridine-6-carbonyl)-3,3-dimethylpiperazin-1-yl)-2,4-dimethylnicotinic acid;
6-(4-(3-(3-fluoro-4-(trifluoromethyl)phenyl)-1-isobutyl-1H-pyrrolo[2,3-b]pyridine-6-carbonyl)-3,3-dimethylpiperazin-1-yl)-2,4-dimethylnicotinic acid;
4-(3-(3,4-dimethoxyphenyl)-1-isobutyl-1H-pyrrolo[2,3-b]pyridine-6-carbonyl)-3,3-dimethylpiperazin-2-one;
6-(4-(1-(tert-butyl)-3-(4-chloro-3-fluorophenyl)-1H-pyrrolo[2,3-b]pyridine-6-carbonyl)-2-(methoxymethyl)piperazin-1-yl)-2,4-dimethylnicotinic acid;
6-((1-(1-(tert-butyl)-3-(4-chloro-3-fluorophenyl)-1H-pyrrolo[2,3-b]pyridine-6-carbonyl)piperidin-4-yl)(methyl)amino)-2,4-dimethylnicotinic acid;
6-((1S,4S)-5-(1-(tert-butyl)-3-(4-chloro-3-fluorophenyl)-1H-pyrrolo[2,3-b]pyridine-6-carbonyl)-2,5-diazabicyclo[2.2.1]heptan-2-yl)-2,4-dimethylnicotinic acid;
6-(4-(3-(4-chloro-3-fluorophenyl)-1-(pyridin-4-ylmethyl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-(pyridin-2-ylmethyl)-1H-pyrrolo[2,3-b]pyridine-6-carbonyl)-3,3-dimethylpiperazin-1-yl)-2,4-dimethylnicotinic acid;
4-(3-(4-chloro-3-fluorophenyl)-1-(1-methoxy-2-methylpropan-2-yl)-1H-pyrrolo[2,3-b]pyridine-6-carbonyl)-3,3-dimethylpiperazin-2-one;
6-(4-(3-(4-chloro-3-fluorophenyl)-1-(1-methoxy-2-methylpropan-2-yl)-1H-pyrrolo[2,3-b]pyridine-6-carbonyl)-3,3-dimethylpiperazin-1-yl)-2,4-dimethylnicotinic acid;
(R)-6-((1-(1-(tert-butyl)-3-(4-chloro-3-fluorophenyl)-1H-pyrrolo[2,3-b]pyridine-6-carbonyl)pyrrolidin-3-yl)amino)-2,4-dimethylnicotinic acid;
(S)-6-((1-(1-(tert-butyl)-3-(4-chloro-3-fluorophenyl)-1H-pyrrolo[2,3-b]pyridine-6-carbonyl)pyrrolidin-3-yl)amino)-2,4-dimethylnicotinic acid;
(R)-6-((1-(1-(tert-butyl)-3-(4-chloro-3-fluorophenyl)-1H-pyrrolo[2,3-b]pyridine-6-carbonyl)pyrrolidin-3-yl)(methyl)amino)-2,4-dimethylnicotinic acid;
(S)-6-((1-(1-(tert-butyl)-3-(4-chloro-3-fluorophenyl)-1H-pyrrolo[2,3-b]pyridine-6-carbonyl)pyrrolidin-3-yl)(methyl)amino)-2,4-dimethylnicotinic acid;
6-(4-(1-(tert-butyl)-3-(4-chloro-3-fluorophenyl)-1H-pyrrolo[2,3-b]pyridine-6-carbonyl)-2-(hydroxymethyl)piperazin-1-yl)-2,4-dimethylnicotinic acid;
6-(4-(3-(4-chloro-3-fluorophenyl)-1-(pyrimidin-2-ylmethyl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-((2,5-dimethyloxazol-4-yl)methyl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-(cyclopropylmethyl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-((6-methoxypyridin-2-yl)methyl)-1H-pyrrolo[2,3-b]pyridine-6-carbonyl)-3,3-dimethylpiperazin-1-yl)-2,4-dimethylnicotinic acid;
1-(1-(tert-butyl)-3-(4-chloro-3-fluorophenyl)-1H-pyrrolo[2,3-b]pyridine-6-carbonyl)piperidine-4-carboxylic acid;
2-(1-(1-(tert-butyl)-3-(4-chloro-3-fluorophenyl)-1H-pyrrolo[2,3-b]pyridine-6-carbonyl)piperidin-4-yl)acetamide;
2-(1-(1-(tert-butyl)-3-(4-chloro-3-fluorophenyl)-1H-pyrrolo[2,3-b]pyridine-6-carbonyl)piperidin-4-yl)-N,N-dimethylacetamide;
(R)-6-(4-(1-(sec-butyl)-3-(4-chloro-3-fluorophenyl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-(1,3-dimethoxypropan-2-yl)-1H-pyrrolo[2,3-b]pyridine-6-carbonyl)-3,3-dimethylpiperazin-1-yl)-2,4-dimethylnicotinic acid;
1′-(1-(tert-butyl)-3-(4-chloro-3-fluorophenyl)-1H-pyrrolo[2,3-b]pyridine-6-carbonyl)-4,6-dimethyl-1′,2′,3′,6′-tetrahydro-[2,4′-bipyridine]-5-carboxylic acid;
6-(1-(1-(tert-butyl)-3-(4-chloro-3-fluorophenyl)-1H-pyrrolo[2,3-b]pyridine-6-carbonyl)piperidin-4-yl)-2,4-dimethylnicotinic acid;
6-(4-(1-(tert-butyl)-3-(4-chloro-3-fluorophenyl)-1H-pyrrolo[2,3-b]pyridine-6-carbonyl)-3,3-dimethylpiperazine-1-carbonyl)nicotinic acid;
6-(4-(1-isobutyl-3-(6-(trifluoromethyl)pyridin-3-yl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-(cyclobutylmethyl)-1H-pyrrolo[2,3-b]pyridine-6-carbonyl)-3,3-dimethylpiperazin-1-yl)-2,4-dimethylnicotinic acid;
(S)-6-(4-(1-(sec-butyl)-3-(4-chloro-3-fluorophenyl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-(cyclobutylmethyl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-isobutyl-5-methyl-1H-pyrrolo[2,3-b]pyridine-6-carbonyl)-3,3-dimethylpiperazin-1-yl)-2,4-dimethylnicotinic acid;
6-(4-(5-(4-chloro-3-fluorophenyl)-7-isobutyl-7H-pyrrolo[2,3-d]pyrimidine-2-carbonyl)-3,3-dimethylpiperazin-1-yl)-2,4-dimethylnicotinic acid;
2-(2-(4-(1-(tert-butyl)-3-(4-chloro-3-fluorophenyl)-1H-pyrrolo[2,3-b]pyridine-6-carbonyl)-3,3-dimethylpiperazin-1-yl)pyridin-4-yl)-2-methylpropanenitrile;
2-(2-(4-(1-(tert-butyl)-3-(4-chloro-3-fluorophenyl)-1H-pyrrolo[2,3-b]pyridine-6-carbonyl)-3,3-dimethylpiperazin-1-yl)pyridin-4-yl)-2-methylpropanamide;
diethyl (6-(4-(1-(tert-butyl)-3-(4-chloro-3-fluorophenyl)-1H-pyrrolo[2,3-b]pyridine-6-carbonyl)-3,3-dimethylpiperazin-1-yl)pyridin-3-yl)phosphonate;
(S)-6-(4-(1-(tert-butyl)-3-(4-chloro-3-fluorophenyl)-1H-pyrrolo[2,3-b]pyridine-6-carbonyl)-3-methylpiperazin-1-yl)-2,4-dimethylnicotinic acid;
6-(4-(3-(4-chloro-3-fluorophenyl)-1-(2-hydroxy-2-methylpropyl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-((3-methyloxetan-3-yl)methyl)-1H-pyrrolo[2,3-b]pyridine-6-carbonyl)-3,3-dimethylpiperazin-1-yl)-2,4-dimethylnicotinic acid;
(S)-6-(4-(3-(4-chloro-3-fluorophenyl)-1-((tetrahydrofuran-3-yl)methyl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-((4-methyltetrahydro-2H-pyran-4-yl)methyl)-1H-pyrrolo[2,3-b]pyridine-6-carbonyl)-3,3-dimethylpiperazin-1-yl)-2,4-dimethylnicotinic acid;
(R)-6-(4-(3-(4-chloro-3-fluorophenyl)-1-((tetrahydrofuran-3-yl)methyl)-1H-pyrrolo[2,3-b]pyridine-6-carbonyl)-3,3-dimethylpiperazin-1-yl)-2,4-dimethylnicotinic acid;
6-(4-(3-(tert-butyl)-1-(4-chloro-3-fluorophenyl)-1H-pyrrolo[3,2-b]pyridine-5-carbonyl)-3,3-dimethylpiperazin-1-yl)-2,4-dimethylnicotinic acid;
6-(4-(3-(4-chloro-3-fluorophenyl)-1-isobutyl-5-methyl-1H-pyrrolo[2,3-b]pyridine-6-carbonyl)piperazin-1-yl)-2,4-dimethylnicotinic acid;
1-((1-(1-(tert-butyl)-3-(4-chloro-3-fluorophenyl)-1H-pyrrolo[2,3-b]pyridine-6-carbonyl)piperidin-4-yl)methyl)-1H-pyrazole-4-carboxylic acid;
6-(4-(3-(4-chloro-3-fluorophenyl)-1-isobutyl-4-methyl-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-isobutyl-4-methyl-1H-pyrrolo[2,3-b]pyridine-6-carbonyl)piperazin-1-yl)-2,4-dimethylnicotinic acid;
6-(4-(7-(4-chloro-3-fluorophenyl)-5-isobutyl-5H-pyrrolo[2,3-b]pyrazine-3-carbonyl)-3,3-dimethylpiperazin-1-yl)-2,4-dimethylnicotinic acid;
6-(4-(1-(tert-butyl)-3-(3,4-difluorophenyl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-((6-methylpyridin-2-yl)methyl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-((3-methylpyridin-2-yl)methyl)-1H-pyrrolo[2,3-b]pyridine-6-carbonyl)-3,3-dimethylpiperazin-1-yl)-2,4-dimethylnicotinic acid;
(6-(4-(1-(tert-butyl)-3-(4-chloro-3-fluorophenyl)-1H-pyrrolo[2,3-b]pyridine-6-carbonyl)-3,3-dimethylpiperazin-1-yl)pyridin-3-yl)phosphonic acid;
ethyl hydrogen (6-(4-(1-(tert-butyl)-3-(4-chloro-3-fluorophenyl)-1H-pyrrolo[2,3-b]pyridine-6-carbonyl)-3,3-dimethylpiperazin-1-yl)pyridin-3-yl)phosphonate;
8-(1-(tert-butyl)-3-(4-chloro-3-fluorophenyl)-1H-pyrrolo[2,3-b]pyridine-6-carbonyl)-3-methyl-1,3,8-triazaspiro[4.5]decane-2,4-dione;
8-(1-(tert-butyl)-3-(4-chloro-3-fluorophenyl)-1H-pyrrolo[2,3-b]pyridine-6-carbonyl)-1,3-dimethyl-1,3,8-triazaspiro[4.5]decane-2,4-dione;
6-((1-(1-(tert-butyl)-3-(4-chloro-3-fluorophenyl)-1H-pyrrolo[2,3-b]pyridine-6-carbonyl)-3-methylpiperidin-4-yl)amino)-2,4-dimethylnicotinic acid;
6-((1-(1-(tert-butyl)-3-(4-chloro-3-fluorophenyl)-1H-pyrrolo[2,3-b]pyridine-6-carbonyl)-3-methylpiperidin-4-yl)(methyl)amino)-2,4-dimethylnicotinic acid;
8-(1-(tert-butyl)-3-(4-chloro-3-fluorophenyl)-1H-pyrrolo[2,3-b]pyridine-6-carbonyl)-6,6-dimethyl-1,3,8-triazaspiro[4.5]decane-2,4-dione;
8-(1-(tert-butyl)-3-(4-chloro-3-fluorophenyl)-1H-pyrrolo[2,3-b]pyridine-6-carbonyl)-6,6-difluoro-1,3,8-triazaspiro[4.5]decane-2,4-dione;
8-(1-(tert-butyl)-3-(4-chloro-3-fluorophenyl)-1H-pyrrolo[2,3-b]pyridine-6-carbonyl)-1,8-diazaspiro[4.5]decan-2-one;
methyl 2-(1-(3-(4-chloro-3-fluorophenyl)-1-isobutyl-1H-indole-6-carbonyl)piperidin-4-yl)acetate;
2-(1-(3-(4-chloro-3-fluorophenyl)-1-isobutyl-1H-indole-6-carbonyl)piperidin-4-yl)acetic acid;
6-(4-(3-(4-chloro-3-fluorophenyl)-1-isobutyl-2-(trifluoromethyl)-1H-pyrrolo[2,3-b]pyridine-6-carbonyl)-3,3-dimethylpiperazin-1-yl)-2,4-dimethylnicotinic acid;
2-(1-(3-acetyl-1-(4-chloro-3-fluorophenyl)-1H-indole-5-carbonyl)piperidin-4-yl)acetic acid;
8-(1-(tert-butyl)-3-(4-chloro-3-fluorophenyl)-1H-pyrrolo[2,3-b]pyridine-6-carbonyl)-1-isopropyl-1,3,8-triazaspiro[4.5]decan-4-one;
5-(4-(1-(tert-butyl)-3-(4-chloro-3-fluorophenyl)-1H-pyrrolo[2,3-b]pyridine-6-carbonyl)piperazin-1-yl)pyridazin-3(2H)-one;
8-(1-(tert-butyl)-3-(4-chloro-3-fluorophenyl)-1H-pyrrolo[2,3-b]pyridine-6-carbonyl)-1-methyl-1,3,8-triazaspiro[4.5]decan-4-one;
4-(4-(1-(tert-butyl)-3-(4-chloro-3-fluorophenyl)-1H-pyrrolo[2,3-b]pyridine-6-carbonyl)-3,3-dimethylpiperazin-1-yl)-2,6-dimethylbenzoic acid;
(S)-6-(4-(3-(4-chloro-3-fluorophenyl)-1-((tetrahydrofuran-2-yl)methyl)-1H-pyrrolo[2,3-b]pyridine-6-carbonyl)-3,3-dimethylpiperazin-1-yl)-2,4-dimethylnicotinic acid;
(R)-6-(4-(3-(4-chloro-3-fluorophenyl)-1-((tetrahydrofuran-2-yl)methyl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-((5-methylpyridin-2-yl)methyl)-1H-pyrrolo[2,3-b]pyridine-6-carbonyl)-3,3-dimethylpiperazin-1-yl)-2,4-dimethylnicotinic acid;
(1-(tert-butyl)-3-(4-chloro-3-fluorophenyl)-1H-pyrrolo[2,3-b]pyridin-6-yl)(1,1-dioxidothiomorpholino)methanone;
8-(4-(1-(tert-butyl)-3-(4-chloro-3-fluorophenyl)-1H-pyrrolo[2,3-b]pyridine-6-carbonyl)-3,3-dimethylpiperazin-1-yl)-8-oxooctanoic acid;
ethyl 6-(4-(8-(tert-butyl)-6-(4-chlorophenyl)imidazo[1,5-a]pyrimidine-2-carbonyl)-3,3-dimethylpiperazin-1-yl)nicotinate;
6-(4-(8-(tert-butyl)-6-(4-chlorophenyl)imidazo[1,5-a]pyrimidine-2-carbonyl)-3,3-dimethylpiperazin-1-yl)nicotinic acid;
ethyl 6-(4-(3-(4-chlorophenyl)-1-isobutylimidazo[1,5-a]pyridine-7-carbonyl)-3,3-dimethylpiperazin-1-yl)nicotinate;
6-(4-(3-(4-chlorophenyl)-1-isobutylimidazo[1,5-a]pyridine-7-carbonyl)-3,3-dimethylpiperazin-1-yl)nicotinic acid;
6-(4-(1-(tert-butyl)-3-(3-fluoro-4-(trifluoromethyl)phenyl)-1H-pyrrolo[2,3-b]pyridine-6-carbonyl)-3,3-dimethylpiperazin-1-yl)-2,4-dimethylnicotinic acid;
4-(3-(tert-butyl)-1-(4-chloro-3-fluorophenyl)-1H-pyrrolo[3,2-b]pyridine-5-carbonyl)-3,3-dimethylpiperazin-2-one;
2-(1-(3-(tert-butyl)-1-(4-chloro-3-fluorophenyl)-1H-pyrrolo[3,2-b]pyridine-5-carbonyl)piperidin-4-yl)acetic acid;
4-(3-(benzo[d][1,3]dioxol-5-yl)-1-isobutyl-1H-pyrrolo[2,3-b]pyridine-6-carbonyl)-3,3-dimethylpiperazin-2-one;
4-(3-(benzofuran-2-yl)-1-isobutyl-1H-pyrrolo[2,3-b]pyridine-6-carbonyl)-3,3-dimethylpiperazin-2-one;
4-(3-(5-fluoro-1H-indol-2-yl)-1-isobutyl-1H-pyrrolo[2,3-b]pyridine-6-carbonyl)-3,3-dimethylpiperazin-2-one;
4-(1-isobutyl-3-(naphthalen-2-yl)-1H-pyrrolo[2,3-b]pyridine-6-carbonyl)-3,3-dimethylpiperazin-2-one;
4-(1-(4-chloro-3-fluorophenyl)-3-isobutyl-1H-indazole-5-carbonyl)-3,3-dimethylpiperazin-2-one;

or a pharmaceutically acceptable salt or solvate of any one of the above-mentioned compounds.

Moreover, it is even more preferred that the compound of formula (I) is selected from:
4-(1-(tert-butyl)-3-(4-chloro-3-fluorophenyl)-1H-pyrrolo[2,3-b]pyridine-6-carbonyl)-3,3-dimethylpiperazin-2-one;
4-(1-(tert-butyl)-3-(4-chloro-3-fluorophenyl)-1H-pyrrolo[2,3-b]pyridine-6-carbonyl)-1,3,3-trimethylpiperazin-2-one;
methyl 2-(4-(1-(tert-butyl)-3-(4-chloro-3-fluorophenyl)-1H-pyrrolo[2,3-b]pyridine-6-carbonyl)-3,3-dimethyl-2-oxopiperazin-1-yl)acetate;
2-(4-(1-(tert-butyl)-3-(4-chloro-3-fluorophenyl)-1H-pyrrolo[2,3-b]pyridine-6-carbonyl)-3,3-dimethyl-2-oxopiperazin-1-yl)acetic acid;
2-(4-(1-(tert-butyl)-3-(4-chloro-3-fluorophenyl)-1H-pyrrolo[2,3-b]pyridine-6-carbonyl)-3,3-dimethyl-2-oxopiperazin-1-yl)acetamide;
2-(4-(1-(tert-butyl)-3-(4-chloro-3-fluorophenyl)-1H-pyrrolo[2,3-b]pyridine-6-carbonyl)-3,3-dimethyl-2-oxopiperazin-1-yl)-N-methylacetamide;
2-(4-(1-(tert-butyl)-3-(4-chloro-3-fluorophenyl)-1H-pyrrolo[2,3-b]pyridine-6-carbonyl)-3,3-dimethyl-2-oxopiperazin-1-yl)-N,N-dimethylacetamide;
4-(1-(tert-butyl)-3-(4-chloro-3-fluorophenyl)-1H-pyrrolo[2,3-b]pyridine-6-carbonyl)-3,3-dimethyl-1-(2-morpholino-2-oxoethyl)piperazin-2-one;
(1-(tert-butyl)-3-(4-chloro-3-fluorophenyl)-1H-pyrrolo[2,3-b]pyridin-6-yl)((1R,3r,5S)-3-hydroxy-8-azabicyclo[3.2.1]octan-8-yl)methanone;
(1-(tert-butyl)-3-(4-chloro-3-fluorophenyl)-1H-pyrrolo[2,3-b]pyridin-6-yl)((1R,3s,5S)-3-hydroxy-8-azabicyclo[3.2.1]octan-8-yl)methanone;
8-(1-(tert-butyl)-3-(4-chloro-3-fluorophenyl)-1H-pyrrolo[2,3-b]pyridine-6-carbonyl)-2,8-diazaspiro[4.5]decan-1-one;
8-(1-(tert-butyl)-3-(4-chloro-3-fluorophenyl)-1H-pyrrolo[2,3-b]pyridine-6-carbonyl)-2,8-diazaspiro[4.5]decan-3-one;
(3aS,6aS)-1-(1-(tert-butyl)-3-(4-chloro-3-fluorophenyl)-1H-pyrrolo[2,3-b]pyridine-6-carbonyl)hexahydropyrrolo[3,4-b]pyrrol-6(1H)-one;
(1-(tert-butyl)-3-(4-chloro-3-fluorophenyl)-1H-pyrrolo[2,3-b]pyridin-6-yl)(2,2-dimethylpyrrolidin-1-yl)methanone;
4-(1-(tert-butyl)-3-(4-chloro-3-fluorophenyl)-1H-pyrrolo[2,3-b]pyridine-6-carbonyl)-1,4-diazepan-2-one;
1-(1-(tertbutyl)-3-(4-chloro-3-fluorophenyl)-1H-pyrrolo[2,3-b]pyridine-6-carbonyl)-1,4-diazepan-5-one;
((1R,5S)-3-oxa-8-azabicyclo[3.2.1]octan-8-yl)(1-(tertbutyl)-3-(4-chloro-3-fluorophenyl)-1H-pyrrolo[2,3-b]pyridin-6-yl)methanone;
4-(1-(tertbutyl)-3-(4-chloro-3-fluorophenyl)-1H-pyrrolo[2,3-b]pyridine-6-carbonyl)-1-methylpiperazin-2-one;
methyl 1-(1-(tert-butyl)-3-(4-chloro-3-fluorophenyl)-1H-pyrrolo[2,3-b]pyridine-6-carbonyl)-3,3-dimethylpiperidine-4-carboxylate;
1-(1-(tert-butyl)-3-(4-chloro-3-fluorophenyl)-1H-pyrrolo[2,3-b]pyridine-6-carbonyl)-3,3-dimethylpiperidine-4-carboxylic acid;
1-(1-(tert-butyl)-3-(4-chloro-3-fluorophenyl)-1H-pyrrolo[2,3-b]pyridine-6-carbonyl)-3,3-dimethylpiperidine-4-carboxamide;
8-(1-(tert-butyl)-3-(4-chloro-3-fluorophenyl)-1H-pyrrolo[2,3-b]pyridine-6-carbonyl)-1,3,8-triazaspiro[4.5]decane-2,4-dione;
methyl 6-(4-(1-(tert-butyl)-3-(4-chloro-3-fluorophenyl)-1H-pyrrolo[2,3-b]pyridine-6-carbonyl)-3,3-dimethylpiperazin-1-yl)-2,4-dimethylnicotinate;
6-(4-(1-(tert-butyl)-3-(4-chloro-3-fluorophenyl)-1H-pyrrolo[2,3-b]pyridine-6-carbonyl)-3,3-dimethylpiperazin-1-yl)-2,4-dimethylnicotinic acid;
4-(3-(4-chloro-3-fluorophenyl)-1-(4-methoxybenzyl)-1H-pyrrolo[2,3-b]pyridine-6-carbonyl)-3,3-dimethylpiperazin-2-one;
4-(3-(4-chloro-3-fluorophenyl)-1-(3-methoxycyclobutyl)-1H-pyrrolo[2,3-b]pyridine-6-carbonyl)-3,3-dimethylpiperazin-2-one;
4-(3-(4-chloro-3-fluorophenyl)-1-isobutyl-1H-pyrrolo[2,3-b]pyridine-6-carbonyl)-3,3-dimethylpiperazin-2-one;
4-(3-(4-chloro-3-fluorophenyl)-1-(tetrahydro-2H-pyran-4-yl)-1H-pyrrolo[2,3-b]pyridine-6-carbonyl)-3,3-dimethylpiperazin-2-one;
4-(3-(4-chloro-3-fluorophenyl)-1-(2,2,2-trifluoroethyl)-1H-pyrrolo[2,3-b]pyridine-6-carbonyl)-3,3-dimethylpiperazin-2-one;
(3-(4-chloro-3-fluorophenyl)-1-(2,2,2-trifluoroethyl)-1H-pyrrolo[2,3-b]pyridin-6-yl)(1,4-oxazepan-4-yl)methanone;
(8-oxa-3-azabicyclo[3.2.1]octan-3-yl)(3-(4-chloro-3-fluorophenyl)-1-(2,2,2-trifluoroethyl)-1H-pyrrolo[2,3-b]pyridin-6-yl)methanone;
4-(3-(4-chloro-3-fluorophenyl)-1-methyl-1H-pyrrolo[2,3-b]pyridine-6-carbonyl)-3,3-dimethylpiperazin-2-one;
4-(3-(4-chloro-3-fluorophenyl)-1-(cyclopropylmethyl)-1H-pyrrolo[2,3-b]pyridine-6-carbonyl)-3,3-dimethylpiperazin-2-one;
4-(3-(4-chloro-3-fluorophenyl)-1-isobutyl-2-methyl-1H-pyrrolo[2,3-b]pyridine-6-carbonyl)-3,3-dimethylpiperazin-2-one;
4-(3-(4-chloro-3-fluorophenyl)-1-(2-(2,2,2-trifluoroethoxy)ethyl)-1H-pyrrolo[2,3-b]pyridine-6-carbonyl)-3,3-dimethylpiperazin-2-one;
4-(1-(2-(1H-1,2,4-triazol-1-yl)ethyl)-3-(4-chloro-3-fluorophenyl)-1H-pyrrolo[2,3-b]pyridine-6-carbonyl)-3,3-dimethylpiperazin-2-one;
4-(3-(4-chloro-3-fluorophenyl)-1-(2-(2-oxopyrrolidin-1-yl)ethyl)-1H-pyrrolo[2,3-b]pyridine-6-carbonyl)-3,3-dimethylpiperazin-2-one;
4-(3-(4-chloro-3-fluorophenyl)-1-(2-ethoxyethyl)-1H-pyrrolo[2,3-b]pyridine-6-carbonyl)-3,3-dimethylpiperazin-2-one;
4-(1-isobutyl-3-(6-(trifluoromethyl)pyridin-3-yl)-1H-pyrrolo[2,3-b]pyridine-6-carbonyl)-3,3-dimethylpiperazin-2-one;
4-(1-isobutyl-3-(1-methyl-1H-pyrazol-4-yl)-1H-pyrrolo[2,3-b]pyridine-6-carbonyl)-3,3-dimethylpiperazin-2-one;
4-(3-(3,6-dihydro-2H-pyran-4-yl)-1-isobutyl-1H-pyrrolo[2,3-b]pyridine-6-carbonyl)-3,3-dimethylpiperazin-2-one;
4-(1-isobutyl-3-(tetrahydro-2H-pyran-4-yl)-1H-pyrrolo[2,3-b]pyridine-6-carbonyl)-3,3-dimethylpiperazin-2-one;
4-(3-(1,2,3,6-tetrahydropyridin-4-yl)-1-isobutyl-1H-pyrrolo[2,3-b]pyridine-6-carbonyl)-3,3-dimethylpiperazin-2-one;
4-(3-cyclohexyl-1-isobutyl-1H-pyrrolo[2,3-b]pyridine-6-carbonyl)-3,3-dimethylpiperazin-2-one;
4-(1-isobutyl-3-phenyl-pyrrolo[2,3-b]pyridine-6-carbonyl)-3,3-dimethyl-piperazin-2-one;
4-[3-(3-chlorophenyl)-1-isobutyl-pyrrolo[2,3-b]pyridine-6-carbonyl]-3,3-dimethyl-piperazin-2-one;
4-[1-isobutyl-3-(p-tolyl)pyrrolo[2,3-b]pyridine-6-carbonyl]-3,3-dimethyl-piperazin-2-one;
4-[1-isobutyl-3-(o-tolyl)pyrrolo[2,3-b]pyridine-6-carbonyl]-3,3-dimethyl-piperazin-2-one;
4-[3-(2-chlorophenyl)-1-isobutyl-pyrrolo[2,3-b]pyridine-6-carbonyl]-3,3-dimethyl-piperazin-2-one;
4-[3-(4-chlorophenyl)-1-isobutyl-pyrrolo[2,3-b]pyridine-6-carbonyl]-3,3-dimethyl-piperazin-2-one;
4-[1-isobutyl-3-[4-(trifluoromethyl)phenyl]pyrrolo[2,3-b]pyridine-6-carbonyl]-3,3-dimethyl-piperazin-2-one;
4-[1-isobutyl-3-[4-fluorophenyl]pyrrolo[2,3-b]pyridine-6-carbonyl]-3,3-dimethyl-piperazin-2-one;
4-[1-isobutyl-3-[3-(trifluoromethoxy)phenyl]pyrrolo[2,3-b]pyridine-6-carbonyl]-3,3-dimethyl-piperazin-2-one;
6-(4-(3-(tert-butyl)-1-(4-chloro-3-fluorophenyl)-1H-pyrazolo[4,3-b]pyridine-5-carbonyl)-3,3-dimethylpiperazin-1-yl)-2,4-dimethylnicotinic acid;
6-(4-(3-(tert-butyl)-1-(4-chloro-3-methoxyphenyl)-1H-pyrazolo[4,3-b]pyridine-5-carbonyl)-3,3-dimethylpiperazin-1-yl)-2,4-dimethylnicotinic acid;
4-(1-(2-(1H-pyrazol-1-yl)ethyl)-3-(4-chloro-3-fluorophenyl)-1H-pyrrolo[2,3-b]pyridine-6-carbonyl)-3,3-dimethylpiperazin-2-one;
4-(1-(4-chloro-3-fluorophenyl)-3-isobutyl-1H-pyrazolo[4,3-b]pyridine-5-carbonyl)-3,3-dimethylpiperazin-2-one;
methyl 6-(4-(1-(4-chloro-3-fluorophenyl)-3-isobutyl-1H-pyrazolo[4,3-b]pyridine-5-carbonyl)-3,3-dimethylpiperazin-1-yl)-2,4-dimethylnicotinate;
6-(4-(1-(4-chloro-3-fluorophenyl)-3-isobutyl-1H-pyrazolo[4,3-b]pyridine-5-carbonyl)-3,3-dimethylpiperazin-1-yl)-2,4-dimethylnicotinic acid;
4-(3-isobutyl-1-(6-(trifluoromethyl)pyridin-3-yl)-1H-pyrazolo[4,3-b]pyridine-5-carbonyl)-3,3-dimethylpiperazin-2-one;
4-(1-(3-chlorophenyl)-3-isobutyl-1H-pyrazolo[4,3-b]pyridine-5-carbonyl)-3,3-dimethylpiperazin-2-one;
methyl 1-(1-(4-chloro-3-fluorophenyl)-3-isobutyl-1H-pyrazolo[4,3-b]pyridine-5-carbonyl)-3,3-dimethylpiperidine-4-carboxylate;
4-(1-(4-chloro-3-fluorophenyl)-3-methyl-1H-pyrazolo[4,3-b]pyridine-5-carbonyl)-3,3-dimethylpiperazin-2-one;
4-(1-(4-chloro-3-fluorophenyl)-3-(tetrahydro-2H-pyran-4-yl)-1H-pyrazolo[4,3-b]pyridine-5-carbonyl)-3,3-dimethylpiperazin-2-one;
4-(1-(2,4-dichlorophenyl)-3-isobutyl-1H-pyrazolo[4,3-b]pyridine-5-carbonyl)-3,3-dimethylpiperazin-2-one;
4-(1-(2-chlorophenyl)-3-isobutyl-1H-pyrazolo[4,3-b]pyridine-5-carbonyl)-3,3-dimethylpiperazin-2-one;
3-(tert-butyl)-1-(4-chloro-3-fluorophenyl)-1H-pyrazolo[4,3-b]pyridine-5-carbonyl)-3,3-dimethylpiperazin-2-one;
4-(1-(4-chloro-3-fluorophenyl)-3-cyclopentyl-1H-pyrazolo[4,3-b]pyridine-5-carbonyl)-3,3-dimethylpiperazin-2-one;
4-(1-(4-chloro-3-fluorophenyl)-3-cyclopropyl-1H-pyrazolo[4,3-b]pyridine-5-carbonyl)-3,3-dimethylpiperazin-2-one;
4-(1-(4-chloro-3-fluorobenzyl)-3-isobutyl-1H-pyrazolo[4,3-b]pyridine-5-carbonyl)-3,3-dimethylpiperazin-2-one;
4-(3-isobutyl-1-(4-(trifluoromethoxy)phenyl)-1H-pyrazolo[4,3-b]pyridine-5-carbonyl)-3,3-dimethylpiperazin-2-one;
4-(3-(tert-butyl)-1-(4-chloro-3-fluorophenyl)-1H-pyrazolo[3,4-b]pyridine-5-carbonyl)-3,3-dimethylpiperazin-2-one;
1-(4-chloro-3-fluorophenyl)-3-isobutyl-6-methyl-1H-pyrazolo[4,3-b]pyridine-5-carbonyl)-3,3-dimethylpiperazin-2-one;
4-(1-isobutyl-3-(piperidin-1-yl)-1H-pyrrolo[2,3-b]pyridine-6-carbonyl)-3,3-dimethylpiperazin-2-one;
4-(3-(4-chloro-3-fluorophenyl)-1-isobutyl-1H-indazole-6-carbonyl)-3,3-dimethylpiperazin-2-one;
4-(3-(2,4-dichlorophenyl)-1-isobutyl-1H-pyrrolo[2,3-b]pyridine-6-carbonyl)-3,3-dimethylpiperazin-2-one;
4-(1-(4-chlorophenyl)-3-isobutyl-1H-pyrazolo[4,3-b]pyridine-5-carbonyl)-3,3-dimethylpiperazin-2-one;
methyl 6-(4-(1-(tert-butyl)-3-(4-chloro-3-fluorophenyl)-1H-pyrazolo[3,4-b]pyridine-6-carbonyl)-3,3-dimethylpiperazin-1-yl)-2,4-dimethylnicotinate;
6-(4-(1-(tert-butyl)-3-(4-chloro-3-fluorophenyl)-1H-pyrazolo[3,4-b]pyridine-6-carbonyl)-3,3-dimethylpiperazin-1-yl)-2,4-dimethylnicotinic acid;
4-(1-(4-chloro-3-fluorophenyl)-3-isobutyl-1H-pyrazolo[4,3-b]pyridine-5-carbonyl)piperazin-2-one;
(3-(4-chloro-3-fluorophenyl)-1-(2,2,2-trifluoroethyl)-1H-pyrazolo[4,3-c]pyridin-6-yl)(1,4-oxazepan-4-yl)methanone;
(8-oxa-3-azabicyclo[3.2.1]octan-3-yl)(3-(4-chloro-3-fluorophenyl)-1-(2,2,2-trifluoroethyl)-1H-pyrazolo[4,3-c]pyridin-6-yl)methanone;
4-(3-isobutyl-1-(6-methoxypyridin-3-yl)-1H-pyrazolo[4,3-b]pyridine-5-carbonyl)-3,3-dimethylpiperazin-2-one;
4-(3-isobutyl-1-(4-methoxyphenyl)-1H-pyrazolo[4,3-b]pyridine-5-carbonyl)-3,3-dimethylpiperazin-2-one;
8-(1-(4-chloro-3-fluorophenyl)-3-isobutyl-1H-pyrazolo[4,3-b]pyridine-5-carbonyl)-1-phenyl-1,3,8-triazaspiro[4.5]decan-4-one;
6-(4-(3-(4-chloro-3-fluorophenyl)-1-isobutyl-1H-pyrrolo[2,3-b]pyridine-6-carbonyl)-3,3-dimethylpiperazin-1-yl)-2,4-dimethylnicotinic acid;
4-(3-(4-chloro-3-fluorophenyl)-1-isopropyl-1H-pyrrolo[2,3-b]pyridine-6-carbonyl)-3,3-dimethylpiperazin-2-one;
6-(4-(3-(4-chloro-3-fluorophenyl)-1-isopropyl-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-(tetrahydro-2H-pyran-4-yl)-1H-pyrrolo[2,3-b]pyridine-6-carbonyl)-3,3-dimethylpiperazin-1-yl)-2,4-dimethylnicotinic acid;
6-(4-(1-(tert-butyl)-3-(4-chloro-3-fluorophenyl)-1H-pyrrolo[2,3-b]pyridine-6-carbonyl)piperazin-1-yl)-2,4-dimethylnicotinic acid;
6-((1-(1-(tert-butyl)-3-(4-chloro-3-fluorophenyl)-1H-pyrrolo[2,3-b]pyridine-6-carbonyl)piperidin-4-yl)amino)-2,4-dimethylnicotinic acid;
6-(6-(1-(tert-butyl)-3-(4-chloro-3-fluorophenyl)-1H-pyrrolo[2,3-b]pyridine-6-carbonyl)-3,6-diazabicyclo[3.2.0]heptan-3-yl)-2,4-dimethylnicotinic acid;
6-(4-(3-(4-chloro-3-fluorophenyl)-1-(1-hydroxy-2-methylpropan-2-yl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-(pyrazin-2-ylmethyl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-isobutyl-2-methyl-1H-pyrrolo[2,3-b]pyridine-6-carbonyl)-3,3-dimethylpiperazin-1-yl)-2,4-dimethylnicotinic acid;
4-(1-isobutyl-3-(6-methoxypyridin-3-yl)-1H-pyrrolo[2,3-b]pyridine-6-carbonyl)-3,3-dimethylpiperazin-2-one;
4-(6-(2,2-dimethyl-3-oxopiperazine-1-carbonyl)-1-isobutyl-1H-pyrrolo[2,3-b]pyridin-3-yl)benzonitrile; 4-(3-(3,4-dichlorophenyl)-1-isobutyl-1H-pyrrolo[2,3-b]pyridine-6-carbonyl)-3,3-dimethylpiperazin-2-one;
4-(3-(3,5-difluorophenyl)-1-isobutyl-1H-pyrrolo[2,3-b]pyridine-6-carbonyl)-3,3-dimethylpiperazin-2-one;
4-(3-(6-(dimethylamino)pyridin-3-yl)-1-isobutyl-1H-pyrrolo[2,3-b]pyridine-6-carbonyl)-3,3-dimethylpiperazin-2-one;
5-(6-(2,2-dimethyl-3-oxopiperazine-1-carbonyl)-1-isobutyl-1H-pyrrolo[2,3-b]pyridin-3-yl)picolinonitrile; 4-(1-isobutyl-3-(4-methoxyphenyl)-1H-pyrrolo[2,3-b]pyridine-6-carbonyl)-3,3-dimethylpiperazin-2-one;
4-(1-isobutyl-3-(2-(trifluoromethoxy)phenyl)-1H-pyrrolo[2,3-b]pyridine-6-carbonyl)-3,3-dimethylpiperazin-2-one;
4-(3-(2-fluorophenyl)-1-isobutyl-1H-pyrrolo[2,3-b]pyridine-6-carbonyl)-3,3-dimethylpiperazin-2-one;
4-(3-(3-fluorophenyl)-1-isobutyl-1H-pyrrolo[2,3-b]pyridine-6-carbonyl)-3,3-dimethylpiperazin-2-one;
4-(3-(4-chloro-2-fluorophenyl)-1-isobutyl-1H-pyrrolo[2,3-b]pyridine-6-carbonyl)-3,3-dimethylpiperazin-2-one;
4-(1-isobutyl-3-(4-(trifluoromethoxy)phenyl)-1H-pyrrolo[2,3-b]pyridine-6-carbonyl)-3,3-dimethylpiperazin-2-one;
3-(6-(2,2-dimethyl-3-oxopiperazine-1-carbonyl)-1-isobutyl-1H-pyrrolo[2,3-b]pyridin-3-yl)benzonitrile;
4-(1-isobutyl-3-(m-tolyl)-1H-pyrrolo[2,3-b]pyridine-6-carbonyl)-3,3-dimethylpiperazin-2-one;
6-(4-(3-(4-chloro-3-fluorophenyl)-1,2-dimethyl-1H-pyrrolo[2,3-b]pyridine-6-carbonyl)-3,3-dimethylpiperazin-1-yl)-2,4-dimethylnicotinic acid;
4-(3-(4-chloro-3-fluorophenyl)-1-(pyridin-4-ylmethyl)-1H-pyrrolo[2,3-b]pyridine-6-carbonyl)-3,3-dimethylpiperazin-2-one;
6-(4-(3-(4-chloro-3-fluorophenyl)-1-(1-methylcyclobutyl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-methyl-1H-pyrrolo[2,3-b]pyridine-6-carbonyl)-3,3-dimethylpiperazin-1-yl)-2,4-dimethylnicotinic acid;
6-(1-(1-(tert-butyl)-3-(4-chloro-3-fluorophenyl)-1H-pyrrolo[2,3-b]pyridine-6-carbonyl)piperidine-4-carboxamido)-2,4-dimethylnicotinic acid;
2-((1R,5S,6s)-3-(1-(tert-butyl)-3-(4-chloro-3-fluorophenyl)-1H-pyrrolo[2,3-b]pyridine-6-carbonyl)-3-azabicyclo[3.1.0]hexan-6-yl)acetic acid;
2-(1-(1-(tert-butyl)-3-(4-chloro-3-fluorophenyl)-1H-pyrrolo[2,3-b]pyridine-6-carbonyl)piperidin-4-yl)acetic acid;
6-(4-(3-(4-chloro-3-fluorophenyl)-1-(1-ethoxy-2-methylpropan-2-yl)-1H-pyrrolo[2,3-b]pyridine-6-carbonyl)-3,3-dimethylpiperazin-1-yl)-2,4-dimethylnicotinic acid;
6-(4-(1-(tert-butyl)-3-(4-chloro-3-fluorophenyl)-1H-pyrrolo[2,3-b]pyridine-6-carbonyl)-1,4-diazepan-1-yl)-2,4-dimethylnicotinic acid;
6-(4-(3-(4-chloro-3-fluorophenyl)-1-ethyl-1H-pyrrolo[2,3-b]pyridine-6-carbonyl)-3,3-dimethylpiperazin-1-yl)-2,4-dimethylnicotinic acid;
(R)-6-(4-(1-(tert-butyl)-3-(4-chloro-3-fluorophenyl)-1H-pyrrolo[2,3-b]pyridine-6-carbonyl)-3-methylpiperazin-1-yl)-2,4-dimethylnicotinic acid;
6-(4-(3-(4-chloro-3-fluorophenyl)-1-(2-(pyridin-2-yl)propan-2-yl)-1H-pyrrolo[2,3-b]pyridine-6-carbonyl)-3,3-dimethylpiperazin-1-yl)-2,4-dimethylnicotinic acid;
6-(4-(1-(tert-butyl)-3-(4-chloro-3-fluorophenyl)-1H-pyrrolo[2,3-b]pyridine-6-carbonyl)-2-(methoxymethyl)piperazin-1-yl)-2,4-dimethylnicotinic acid;
6-(4-(1-(carboxymethyl)-3-(4-chloro-3-fluorophenyl)-1H-pyrrolo[2,3-b]pyridine-6-carbonyl)-3,3-dimethylpiperazin-1-yl)-2,4-dimethylnicotinic acid;
6-(4-(3-(3-chloro-4-fluorophenyl)-1-(2-methoxyethyl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-(pyridin-3-ylmethyl)-1H-pyrrolo[2,3-b]pyridine-6-carbonyl)-3,3-dimethylpiperazin-1-yl)-2,4-dimethylnicotinic acid;
(S)-6-(4-(3-(4-chloro-3-fluorophenyl)-1-(1-methoxypropan-2-yl)-1H-pyrrolo[2,3-b]pyridine-6-carbonyl)-3,3-dimethylpiperazin-1-yl)-2,4-dimethylnicotinic acid;
6-(4-(1-(tert-butyl)-3-(4-chloro-3-fluorophenyl)-1H-pyrrolo[2,3-b]pyridine-6-carbonyl)-3,3-dimethylpiperazin-1-yl)nicotinic acid;
6-(4-(1-(tert-butyl)-3-(4-chloro-3-fluorophenyl)-1H-pyrrolo[2,3-b]pyridine-6-carbonyl)-3,3-dimethylpiperazin-1-yl)nicotinamide;
6-(4-(1-(tert-butyl)-3-(4-chloro-3-fluorophenyl)-1H-pyrrolo[2,3-b]pyridine-6-carbonyl)-3,3-dimethylpiperazin-1-yl)-N,N-dimethylnicotinamide;
(S)-6-(4-(3-(4-chloro-3-fluorophenyl)-1-(1-methoxypropan-2-yl)-1H-pyrrolo[2,3-b]pyridine-6-carbonyl)-3,3-dimethylpiperazin-1-yl)-2,4-dimethylnicotinic acid;
6-(4-(3-(3,4-difluorophenyl)-1-isobutyl-1H-pyrrolo[2,3-b]pyridine-6-carbonyl)-3,3-dimethylpiperazin-1-yl)-2,4-dimethylnicotinic acid;
6-(4-(3-(3-fluoro-4-methylphenyl)-1-isobutyl-1H-pyrrolo[2,3-b]pyridine-6-carbonyl)-3,3-dimethylpiperazin-1-yl)-2,4-dimethylnicotinic acid;
6-(4-(1-isobutyl-3-(3,4,5-trifluorophenyl)-1H-pyrrolo[2,3-b]pyridine-6-carbonyl)-3,3-dimethylpiperazin-1-yl)-2,4-dimethylnicotinic acid;
6-(4-(3-(3-fluoro-4-(trifluoromethyl)phenyl)-1-isobutyl-1H-pyrrolo[2,3-b]pyridine-6-carbonyl)-3,3-dimethylpiperazin-1-yl)-2,4-dimethylnicotinic acid;
4-(3-(3,4-dimethoxyphenyl)-1-isobutyl-1H-pyrrolo[2,3-b]pyridine-6-carbonyl)-3,3-dimethylpiperazin-2-one;
6-(4-(1-(tert-butyl)-3-(4-chloro-3-fluorophenyl)-1H-pyrrolo[2,3-b]pyridine-6-carbonyl)-2-(methoxymethyl)piperazin-1-yl)-2,4-dimethylnicotinic acid;
6-((1-(1-(tert-butyl)-3-(4-chloro-3-fluorophenyl)-1H-pyrrolo[2,3-b]pyridine-6-carbonyl)piperidin-4-yl)(methyl)amino)-2,4-dimethylnicotinic acid;
6-((1S,4S)-5-(1-(tert-butyl)-3-(4-chloro-3-fluorophenyl)-1H-pyrrolo[2,3-b]pyridine-6-carbonyl)-2,5-diazabicyclo[2.2.1]heptan-2-yl)-2,4-dimethylnicotinic acid;
6-(4-(3-(4-chloro-3-fluorophenyl)-1-(pyridin-4-ylmethyl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-(pyridin-2-ylmethyl)-1H-pyrrolo[2,3-b]pyridine-6-carbonyl)-3,3-dimethylpiperazin-1-yl)-2,4-dimethylnicotinic acid;
4-(3-(4-chloro-3-fluorophenyl)-1-(1-methoxy-2-methylpropan-2-yl)-1H-pyrrolo[2,3-b]pyridine-6-carbonyl)-3,3-dimethylpiperazin-2-one;
6-(4-(3-(4-chloro-3-fluorophenyl)-1-(1-methoxy-2-methylpropan-2-yl)-1H-pyrrolo[2,3-b]pyridine-6-carbonyl)-3,3-dimethylpiperazin-1-yl)-2,4-dimethylnicotinic acid;
(R)-6-((1-(1-(tert-butyl)-3-(4-chloro-3-fluorophenyl)-1H-pyrrolo[2,3-b]pyridine-6-carbonyl)pyrrolidin-3-yl)(methyl)amino)-2,4-dimethylnicotinic acid;
(S)-6-((1-(1-(tert-butyl)-3-(4-chloro-3-fluorophenyl)-1H-pyrrolo[2,3-b]pyridine-6-carbonyl)pyrrolidin-3-yl)(methyl)amino)-2,4-dimethylnicotinic acid;
6-(4-(1-(tert-butyl)-3-(4-chloro-3-fluorophenyl)-1H-pyrrolo[2,3-b]pyridine-6-carbonyl)-2-(hydroxymethyl)piperazin-1-yl)-2,4-dimethylnicotinic acid;
6-(4-(3-(4-chloro-3-fluorophenyl)-1-(pyrimidin-2-ylmethyl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-((2,5-dimethyloxazol-4-yl)methyl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-(cyclopropylmethyl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-((6-methoxypyridin-2-yl)methyl)-1H-pyrrolo[2,3-b]pyridine-6-carbonyl)-3,3-dimethylpiperazin-1-yl)-2,4-dimethylnicotinic acid;
1-(1-(tert-butyl)-3-(4-chloro-3-fluorophenyl)-1H-pyrrolo[2,3-b]pyridine-6-carbonyl)piperidine-4-carboxylic acid;
2-(1-(1-(tert-butyl)-3-(4-chloro-3-fluorophenyl)-1H-pyrrolo[2,3-b]pyridine-6-carbonyl)piperidin-4-yl)acetamide;
2-(1-(1-(tert-butyl)-3-(4-chloro-3-fluorophenyl)-1H-pyrrolo[2,3-b]pyridine-6-carbonyl)piperidin-4-yl)-N,N-dimethylacetamide;
(R)-6-(4-(1-(sec-butyl)-3-(4-chloro-3-fluorophenyl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-(1,3-dimethoxypropan-2-yl)-1H-pyrrolo[2,3-b]pyridine-6-carbonyl)-3,3-dimethylpiperazin-1-yl)-2,4-dimethylnicotinic acid;
1′-(1-(tert-butyl)-3-(4-chloro-3-fluorophenyl)-1H-pyrrolo[2,3-b]pyridine-6-carbonyl)-4,6-dimethyl-1′,2′,3′,6′-tetrahydro-[2,4′-bipyridine]-5-carboxylic acid;
6-(1-(1-(tert-butyl)-3-(4-chloro-3-fluorophenyl)-1H-pyrrolo[2,3-b]pyridine-6-carbonyl)piperidin-4-yl)-2,4-dimethylnicotinic acid;
6-(4-(1-(tert-butyl)-3-(4-chloro-3-fluorophenyl)-1H-pyrrolo[2,3-b]pyridine-6-carbonyl)-3,3-dimethylpiperazine-1-carbonyl)nicotinic acid;
6-(4-(1-isobutyl-3-(6-(trifluoromethyl)pyridin-3-yl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-(cyclobutylmethyl)-1H-pyrrolo[2,3-b]pyridine-6-carbonyl)-3,3-dimethylpiperazin-1-yl)-2,4-dimethylnicotinic acid;
(S)-6-(4-(1-(sec-butyl)-3-(4-chloro-3-fluorophenyl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-(cyclobutylmethyl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-isobutyl-5-methyl-1H-pyrrolo[2,3-b]pyridine-6-carbonyl)-3,3-dimethylpiperazin-1-yl)-2,4-dimethylnicotinic acid;
6-(4-(5-(4-chloro-3-fluorophenyl)-7-isobutyl-7H-pyrrolo[2,3-d]pyrimidine-2-carbonyl)-3,3-dimethyl piperazin-1-yl)-2,4-dimethylnicotinic acid;
2-(2-(4-(1-(tert-butyl)-3-(4-chloro-3-fluorophenyl)-1H-pyrrolo[2,3-b]pyridine-6-carbonyl)-3,3-dimethylpiperazin-1-yl)pyridin-4-yl)-2-methylpropanenitrile;
2-(2-(4-(1-(tert-butyl)-3-(4-chloro-3-fluorophenyl)-1H-pyrrolo[2,3-b]pyridine-6-carbonyl)-3,3-dimethylpiperazin-1-yl)pyridin-4-yl)-2-methylpropanamide;
diethyl (6-(4-(1-(tert-butyl)-3-(4-chloro-3-fluorophenyl)-1H-pyrrolo[2,3-b]pyridine-6-carbonyl)-3,3-dimethylpiperazin-1-yl)pyridin-3-yl)phosphonate;
(S)-6-(4-(1-(tert-butyl)-3-(4-chloro-3-fluorophenyl)-1H-pyrrolo[2,3-b]pyridine-6-carbonyl)-3-methylpiperazin-1-yl)-2,4-dimethylnicotinic acid;
6-(4-(3-(4-chloro-3-fluorophenyl)-1-(2-hydroxy-2-methylpropyl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-((3-methyloxetan-3-yl)methyl)-1H-pyrrolo[2,3-b]pyridine-6-carbonyl)-3,3-dimethylpiperazin-1-yl)-2,4-dimethylnicotinic acid;
(S)-6-(4-(3-(4-chloro-3-fluorophenyl)-1-((tetrahydrofuran-3-yl)methyl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-((4-methyltetrahydro-2H-pyran-4-yl)methyl)-1H-pyrrolo[2,3-b]pyridine-6-carbonyl)-3,3-dimethylpiperazin-1-yl)-2,4-dimethylnicotinic acid;
(R)-6-(4-(3-(4-chloro-3-fluorophenyl)-1-((tetrahydrofuran-3-yl)methyl)-1H-pyrrolo[2,3-b]pyridine-6-carbonyl)-3,3-dimethylpiperazin-1-yl)-2,4-dimethylnicotinic acid;
6-(4-(3-(tert-butyl)-1-(4-chloro-3-fluorophenyl)-1H-pyrrolo[3,2-b]pyridine-5-carbonyl)-3,3-dimethylpiperazin-1-yl)-2,4-dimethylnicotinic acid;
1-((1-(1-(tert-butyl)-3-(4-chloro-3-fluorophenyl)-1H-pyrrolo[2,3-b]pyridine-6-carbonyl)piperidin-4-yl)methyl)-1H-pyrazole-4-carboxylic acid;
6-(4-(3-(4-chloro-3-fluorophenyl)-1-isobutyl-4-methyl-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-isobutyl-4-methyl-1H-pyrrolo[2,3-b]pyridine-6-carbonyl)piperazin-1-yl)-2,4-dimethylnicotinic acid;
6-(4-(7-(4-chloro-3-fluorophenyl)-5-isobutyl-5H-pyrrolo[2,3-b]pyrazine-3-carbonyl)-3,3-dimethylpiperazin-1-yl)-2,4-dimethylnicotinic acid;
6-(4-(1-(tert-butyl)-3-(3,4-difluorophenyl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-((6-methylpyridin-2-yl)methyl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-((3-methylpyridin-2-yl)methyl)-1H-pyrrolo[2,3-b]pyridine-6-carbonyl)-3,3-dimethylpiperazin-1-yl)-2,4-dimethylnicotinic acid;
(6-(4-(1-(tert-butyl)-3-(4-chloro-3-fluorophenyl)-1H-pyrrolo[2,3-b]pyridine-6-carbonyl)-3,3-dimethylpiperazin-1-yl)pyridin-3-yl)phosphonic acid;
ethyl hydrogen (6-(4-(1-(tert-butyl)-3-(4-chloro-3-fluorophenyl)-1H-pyrrolo[2,3-b]pyridine-6-carbonyl)-3,3-dimethylpiperazin-1-yl)pyridin-3-yl)phosphonate;
8-(1-(tert-butyl)-3-(4-chloro-3-fluorophenyl)-1H-pyrrolo[2,3-b]pyridine-6-carbonyl)-3-methyl-1,3,8-triazaspiro[4.5]decane-2,4-dione;
8-(1-(tert-butyl)-3-(4-chloro-3-fluorophenyl)-1H-pyrrolo[2,3-b]pyridine-6-carbonyl)-1,3-dimethyl-1,3,8-triazaspiro[4.5]decane-2,4-dione;
6-((1-(1-(tert-butyl)-3-(4-chloro-3-fluorophenyl)-1H-pyrrolo[2,3-b]pyridine-6-carbonyl)-3-methylpiperidin-4-yl)amino)-2,4-dimethylnicotinic acid;
6-((1-(1-(tert-butyl)-3-(4-chloro-3-fluorophenyl)-1H-pyrrolo[2,3-b]pyridine-6-carbonyl)-3-methylpiperidin-4-yl)(methyl)amino)-2,4-dimethylnicotinic acid;
8-(1-(tert-butyl)-3-(4-chloro-3-fluorophenyl)-1H-pyrrolo[2,3-b]pyridine-6-carbonyl)-6,6-dimethyl-1,3,8-triazaspiro[4.5]decane-2,4-dione;
8-(1-(tert-butyl)-3-(4-chloro-3-fluorophenyl)-1H-pyrrolo[2,3-b]pyridine-6-carbonyl)-6,6-difluoro-1,3,8-triazaspiro[4.5]decane-2,4-dione;
8-(1-(tert-butyl)-3-(4-chloro-3-fluorophenyl)-1H-pyrrolo[2,3-b]pyridine-6-carbonyl)-1,8-diazaspiro[4.5]decan-2-one;
methyl 2-(1-(3-(4-chloro-3-fluorophenyl)-1-isobutyl-1H-indole-6-carbonyl)piperidin-4-yl)acetate;
2-(1-(3-(4-chloro-3-fluorophenyl)-1-isobutyl-1H-indole-6-carbonyl)piperidin-4-yl)acetic acid;
6-(4-(3-(4-chloro-3-fluorophenyl)-1-isobutyl-2-(trifluoromethyl)-1H-pyrrolo[2,3-b]pyridine-6-carbonyl)-3,3-dimethylpiperazin-1-yl)-2,4-dimethylnicotinic acid;
2-(1-(3-acetyl-1-(4-chloro-3-fluorophenyl)-1H-indole-5-carbonyl)piperidin-4-yl)acetic acid;
8-(1-(tert-butyl)-3-(4-chloro-3-fluorophenyl)-1H-pyrrolo[2,3-b]pyridine-6-carbonyl)-1-isopropyl-1,3,8-triazaspiro[4.5]decan-4-one;
5-(4-(1-(tert-butyl)-3-(4-chloro-3-fluorophenyl)-1H-pyrrolo[2,3-b]pyridine-6-carbonyl)piperazin-1-yl)pyridazin-3(2H)-one;
8-(1-(tert-butyl)-3-(4-chloro-3-fluorophenyl)-1H-pyrrolo[2,3-b]pyridine-6-carbonyl)-1-methyl-1,3,8-triazaspiro[4.5]decan-4-one;
4-(4-(1-(tert-butyl)-3-(4-chloro-3-fluorophenyl)-1H-pyrrolo[2,3-b]pyridine-6-carbonyl)-3,3-dimethylpiperazin-1-yl)-2,6-dimethylbenzoic acid;
(S)-6-(4-(3-(4-chloro-3-fluorophenyl)-1-((tetrahydrofuran-2-yl)methyl)-1H-pyrrolo[2,3-b]pyridine-6-carbonyl)-3,3-dimethylpiperazin-1-yl)-2,4-dimethylnicotinic acid;
(R)-6-(4-(3-(4-chloro-3-fluorophenyl)-1-((tetrahydrofuran-2-yl)methyl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-((5-methylpyridin-2-yl)methyl)-1H-pyrrolo[2,3-b]pyridine-6-carbonyl)-3,3-dimethylpiperazin-1-yl)-2,4-dimethylnicotinic acid;
(1-(tert-butyl)-3-(4-chloro-3-fluorophenyl)-1H-pyrrolo[2,3-b]pyridin-6-yl)(1,1-dioxidothiomorpholino)methanone;
8-(4-(1-(tert-butyl)-3-(4-chloro-3-fluorophenyl)-1H-pyrrolo[2,3-b]pyridine-6-carbonyl)-3,3-dimethylpiperazin-1-yl)-8-oxooctanoic acid;
ethyl 6-(4-(8-(tert-butyl)-6-(4-chlorophenyl)imidazo[1,5-a]pyrimidine-2-carbonyl)-3,3-dimethylpiperazin-1-yl)nicotinate;
6-(4-(8-(tert-butyl)-6-(4-chlorophenyl)imidazo[1,5-a]pyrimidine-2-carbonyl)-3,3-dimethylpiperazin-1-yl)nicotinic acid;
ethyl 6-(4-(3-(4-chlorophenyl)-1-isobutylimidazo[1,5-a]pyridine-7-carbonyl)-3,3-dimethylpiperazin-1-yl)nicotinate;
6-(4-(3-(4-chlorophenyl)-1-isobutylimidazo[1,5-a]pyridine-7-carbonyl)-3,3-dimethylpiperazin-1-yl)nicotinic acid;
6-(4-(1-(tert-butyl)-3-(3-fluoro-4-(trifluoromethyl)phenyl)-1H-pyrrolo[2,3-b]pyridine-6-carbonyl)-3,3-dimethylpiperazin-1-yl)-2,4-dimethylnicotinic acid;
4-(3-(tert-butyl)-1-(4-chloro-3-fluorophenyl)-1H-pyrrolo[3,2-b]pyridine-5-carbonyl)-3,3-dimethylpiperazin-2-one;
2-(1-(3-(tert-butyl)-1-(4-chloro-3-fluorophenyl)-1H-pyrrolo[3,2-b]pyridine-5-carbonyl)piperidin-4-yl)acetic acid;
4-(3-(benzo[d][1,3]dioxol-5-yl)-1-isobutyl-1H-pyrrolo[2,3-b]pyridine-6-carbonyl)-3,3-dimethylpiperazin-2-one;
4-(3-(benzofuran-2-yl)-1-isobutyl-1H-pyrrolo[2,3-b]pyridine-6-carbonyl)-3,3-dimethylpiperazin-2-one;
4-(3-(5-fluoro-1H-indol-2-yl)-1-isobutyl-1H-pyrrolo[2,3-b]pyridine-6-carbonyl)-3,3-dimethylpiperazin-2-one;
4-(1-isobutyl-3-(naphthalen-2-yl)-1H-pyrrolo[2,3-b]pyridine-6-carbonyl)-3,3-dimethylpiperazin-2-one;
4-(1-(4-chloro-3-fluorophenyl)-3-isobutyl-1H-indazole-5-carbonyl)-3,3-dimethylpiperazin-2-one;

or a pharmaceutically acceptable salt or solvate of any one of the above-mentioned compounds.

The present invention also relates to each of the intermediates described further below in the examples section of this specification, including any one of these intermediates in non-salt form and/or non-solvated form, or in the form of a salt or solvate (e.g., a pharmaceutically acceptable salt or solvate) of the respective compound. Such intermediates can be used, in particular, in the synthesis of the compounds of formula (I).

For a person skilled in the field of synthetic chemistry, various ways for the preparation of the compounds of general formula (I) and their pharmaceutically acceptable salts and solvates will be readily apparent. For example, the compounds of the invention can be prepared in accordance with, or in analogy to, the synthetic routes described in detail in the examples section. In particular, the compounds of formula (I) can be synthesized in accordance with the methods described in the following general schemes (general disconnections).

The desired compounds, which are referred to as compounds O in the following, can be prepared through various sequences including the one depicted in the following scheme:

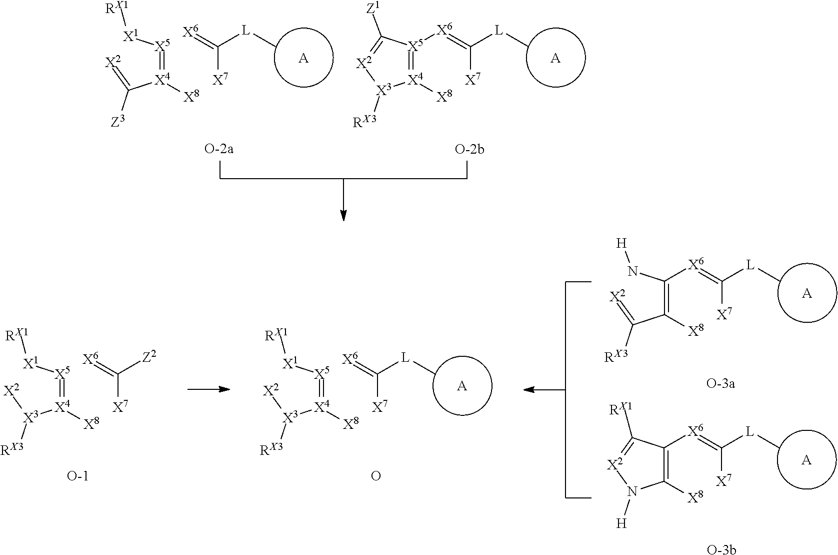

### General Compound O

General compound O can be obtained from a precursor O-1 according to the general disconnection 1:

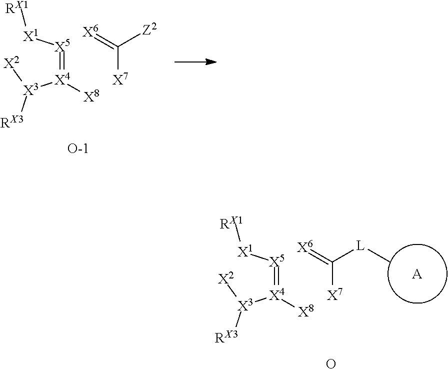

With Z² being a hydrogen atom (based on the work described in ARKIVOC 2013, 1, 154-174, ARKIVOC 2001, 1, 242-268 and Synthesis 2011, 20, 3209-3219):
By N-oxidation followed by a sequence leading to the carboxylic, sulfonic or sulfinic acid and subsequent amide, sulfonamide or sulfinamide synthesis with the appropriate amine.
By N-oxidation followed by a sequence leading to the conversion of Z¹ into a halogen or a pseudo-halogen.
By carbon-hydrogen bond activation with a metal leading to the conversion of Z¹ into a halogen, a pseudo-halogen, or an organometallic group.

With Z² being a hydroxy group, based on the work described in Org. Process Res. Dev. 2004, 8, 62-71 and Tet. Lett., 1992, 33, 1181-1184:
By a transformation or sequence known to the person skilled in the art of Z¹ into a halogen or pseudo-halogen, followed by a transformation or a sequence described below.

With Z² being a halogen or a pseudo-halogen, or an organometallic group:
By a direct metal catalyzed amino-carbonylation reaction, sulfinamide or sulfonamide synthesis with the appropriate amine (based on the work described in RSC Adv., 2014, 4, 10367-10389 and Synthesis, 2008, 311-312.)
By a transformation or a sequence known to the person skilled in the art leading to a carboxylic, sulfonic or sulfinic acid, followed by amide, sulfonamide or sulfinimide synthesis with the appropriate amine (based on the work described in J. Org. Chem., 2008, 73, 3967-3969)

General compound O can be obtained from a precursor O-2a or O-2b according to the general disconnection 2:

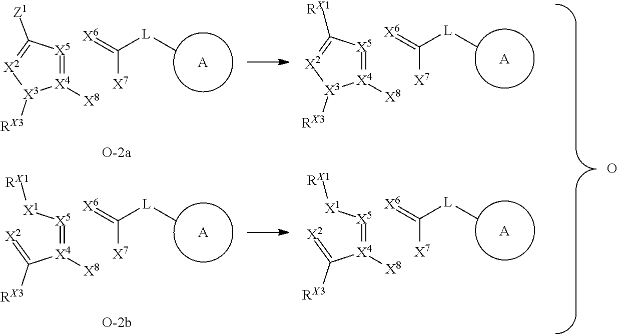

With Z¹/Z³ being a hydrogen atom (based on the work described in J. Org. Chem. 2020, 85, 2, 1009-102, and Angew. Chem. Int. Ed. 2017, 56, 15644-15648):
By carbon-hydrogen bond activation with a metal followed by a transformation or sequence known to the person skilled in the art leading to Z² being a halogen, pseudo-halogen or organometallic group followed by a carbon-carbon or carbon heteroatom coupling reaction with the appropriate coupling partner.

With Z¹/Z³ being a hydroxy group (based on the work described in Bioorg. Med. Chem. Lett. 1990, 9, 3217-3220):
By a transformation or sequence known to the person skilled in the art of Z² into a halogen, pseudo-halogen or organometallic group followed by a carbon-carbon or carbon heteroatom coupling reaction with the appropriate coupling partner.

With Z¹/Z³ being a halogen, pseudo-halogen, or organometallic group (based on the work described in Angew. Chem. Int. Ed. 2017, 56, 7242-7246 and Org. Process Res. Dev. 2019, 23, 8, 1725-1739):
By a carbon-carbon or carbon heteroatom coupling reaction with the appropriate coupling partner.

General compound O can be obtained from a precursor O-3a or O-3b according to the general disconnection 3:

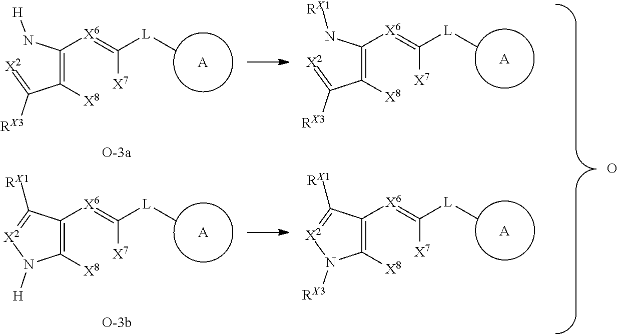

By a N-derivation known to the person skilled in the art, with the appropriate R^{X1} or R^{X3} fragment. (based on the works described in J. Org. Chem. 2004, 69, 6514, Org. Lett. 2000, 2, 1403-1406, J. Org. Chem. 2007, 72, 8943-8946 and J. Med. Chem. 2009, 52, 6527-6530)

General compound O can also be obtained from various precursors according to the general disconnection 4:

From O-4a or O-4b:

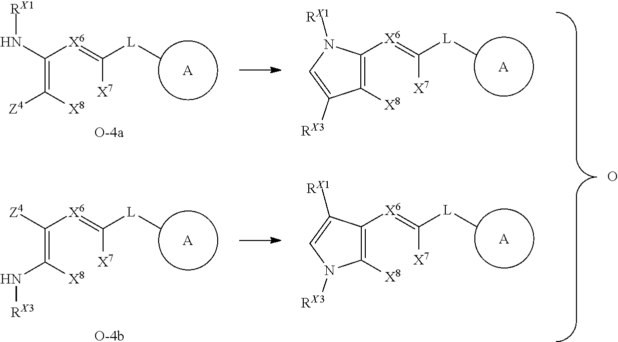

With Z⁴ being a halogen or a pseudo-halogen:
By a sequential condensation with an appropriate aldehyde then intramolecular coupling reaction (based on the works described in Tetrahedron, 2011, 67, 7195-7210).
By a sequential intermolecular coupling reaction with an alkene then intermolecular coupling reaction (based on the works described in Tetrahedron, 2011, 67, 7195-7210).

With Z⁴ being a hydrogen atom:
By simultaneous coupling and cyclisation with an appropriate alkyne (based on the work described in Chem. Commun., 2015, 51, 11202-11205)

From O-5a or O-5b:

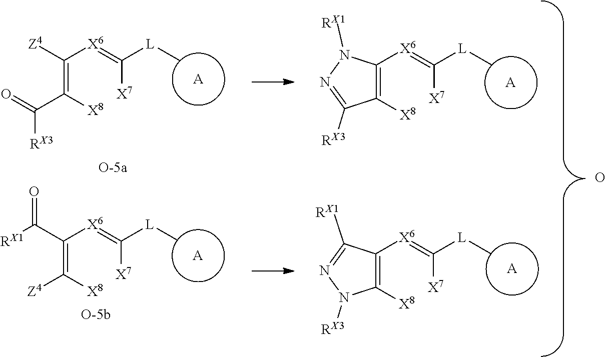

With Z⁴ being a halogen or pseudo-halogen:
By coupling with an appropriate amine followed by cyclisation (based on the work described in Org. Lett., 2010, 12, 4576-4570)
By condensation of an appropriate hydrazine derivative followed by cyclisation (based on the work described in Molecules 2018, 23, 674)
By coupling with an appropriate hydrazine derivative followed by cyclisation (based on the work described in Org. Lett. 2012, 14, 2552-2555)

With Z⁴ being a hydrogen atom:
By condensation of an appropriate hydrazine followed by oxidative cyclisation (based on the work described in Chem. Eur. J. 2014, 20, 3932-3938 and Molecules 2018, 23, 2783)

From O-6a or O-6b:

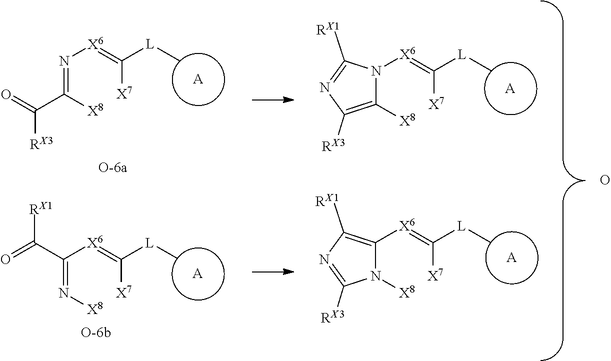

By sequential condensation with an appropriate amine and cyclisation (based on the work described in Org. Biomol. Chem., 2018, 16, 5653-5660).

From O-7a or O-7b:

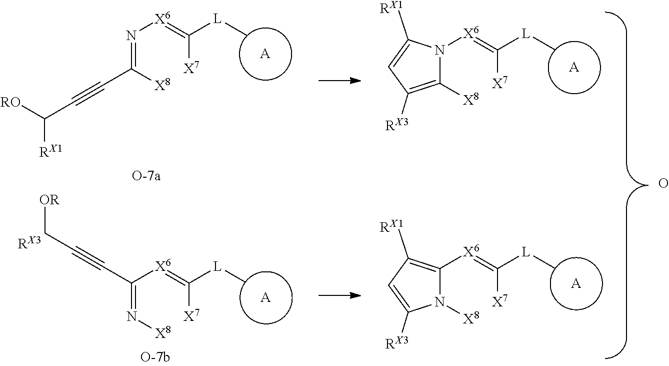

By addition of an appropriate organometallic and subsequent cyclisation (based on the work described in Org. Lett., 2008, 10, 2307-2310)

Some more specific compounds O can be obtained by particular pathways:

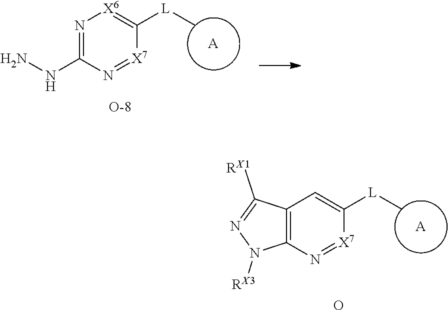

From O-8, by condensation with an appropriate ynone and subsequent Diels-Alder, Retro-Diels-Alder sequence (based on the work described in J. Am. Chem. Soc. 2019, 141, 15901-15909).

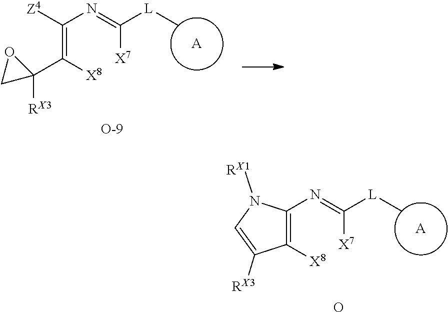

From O-9, with Z⁴ being a halogen or pseudo halogen, by reaction with an appropriate primary amine (based on the work described in J. Org. Chem., 2006, 71, 5538-5545)

### Intermediate O-1

The intermediate O-1 can be obtained from multiple precursors including:

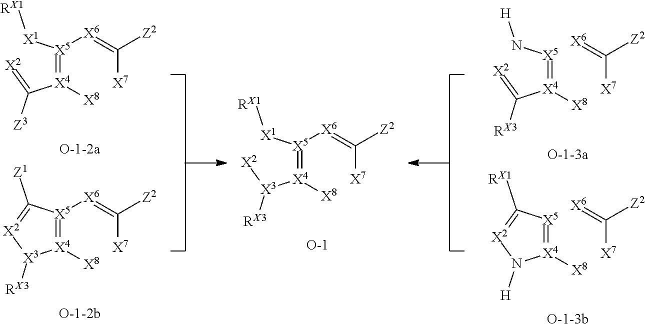

Through the above-mentioned general disconnection 2 from O-1-2a or O-1-2b.

Through the above-mentioned general disconnection 3 from O-1-3a or O-1-3b.

More specific examples of intermediate O-1 can be obtained following the above-mentioned general disconnections 4 from precursors O-1-4-a/b, O-1-5-a/b, O-1-6-a/b, O-1-7-a/b, O-1-8, or O-1-9:

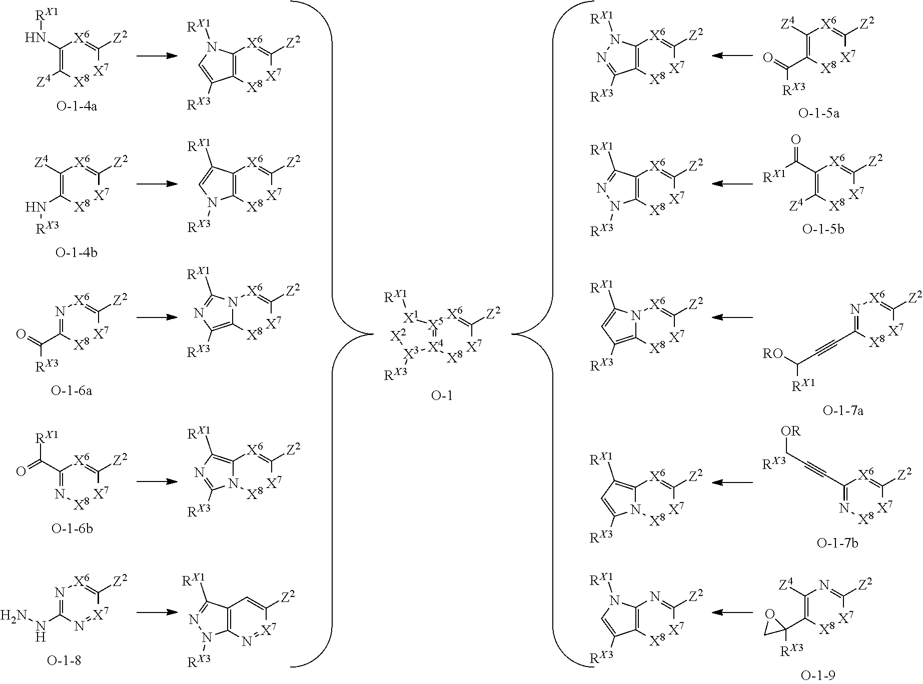

Additionally, some intermediates O-1 can be obtained by other specific pathways including the one described in the following general disconnection 5.

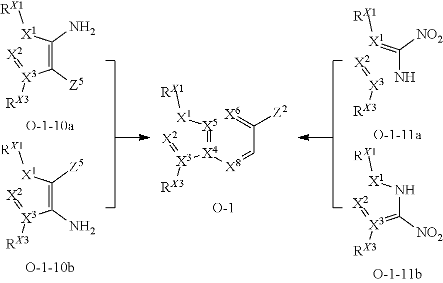

From O-1-10a/b with Z⁵ being a halogen or pseudo halogen:
By sequential coupling with an appropriate alkene and intramolecular cyclisation (based on the work described in Bull. Chem. Soc. J. 1991, 65, 340-344)

From O-1-11a/b:
By Michael addition on an appropriate alkyne, followed by reduction of the nitro group by methods known to the person skilled in the art and intramolecular cyclisation (based on the work described in New J. Chem., 2019, 43, 9961-9968).

Additionally, some other intermediates O-1 can be obtained by other specific pathways.

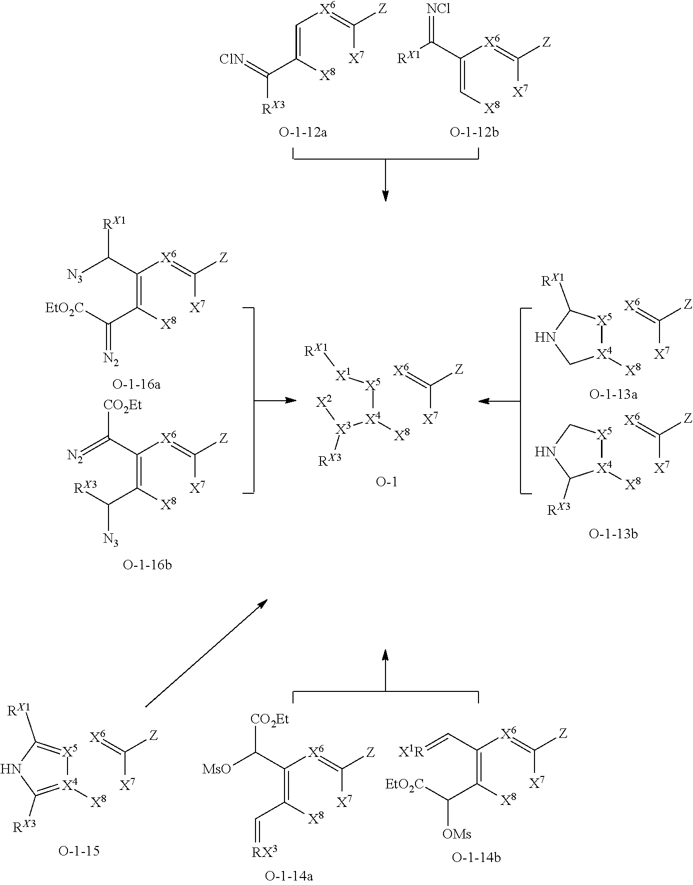

From O-1-12a/b:
By a directed C—H functionalization with an appropriate diazo-phosphono-acetate (based on the work described in Org. Lett. 2019, 17, 6860-6863).

From O-1-13a/b:
By a dehydrogenative coupling reaction, with an appropriate aryl-halide (based on the work described in Org. Lett. 2011, 5, 1238-1241)

From O-1-14a/b:
By sequential nucleophilic displacement with sodium azide, [3+2] cycloaddition and diazote extrusion (based on the work described in Org. Lett. 2009, 11, 729-732)

From O-1-15:
By N-alkylation as described in general disconnection 3

From O-1-16a/b:
By a metal catalyzed, intramolecular nucleophilic attack of azide on a carbene, followed by diazote extrusion (based on the work described in J. Org. Chem. 2019, 84, 5813-5820)

### Intermediate O-2

The intermediates O-2a/b can be obtained from multiple precursors including:

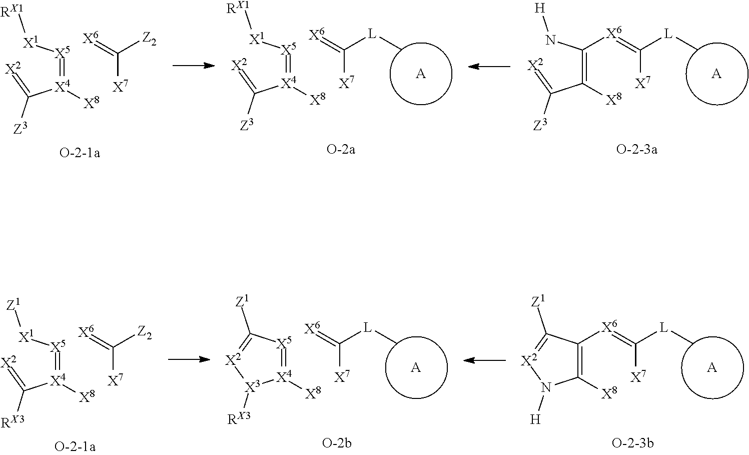

Through the above-mentioned general disconnection 1 from O-2-1a/b.

Through the above-mentioned general disconnection 3 from O-2-3a/b.

More specific examples of intermediate O-2 can be obtained following the above-mentioned general disconnections 4 from precursors O-2-4-a/b, O-2-5-a/b, O-2-6-a/b, O-2-7-a/b, O-2-8, or O-2-9:

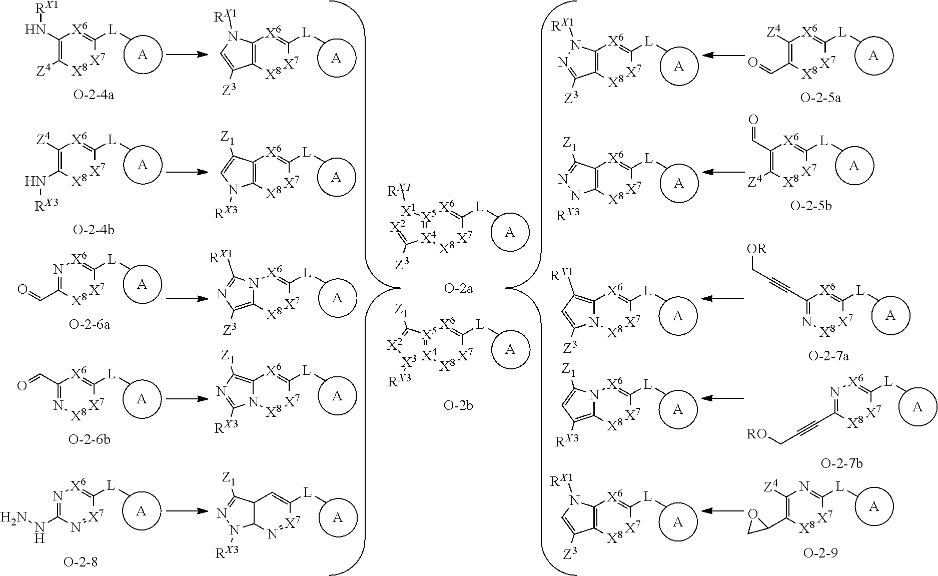

Additionally, some intermediates O-2 can be obtained by other specific pathways including the one described in the following general disconnection 6.

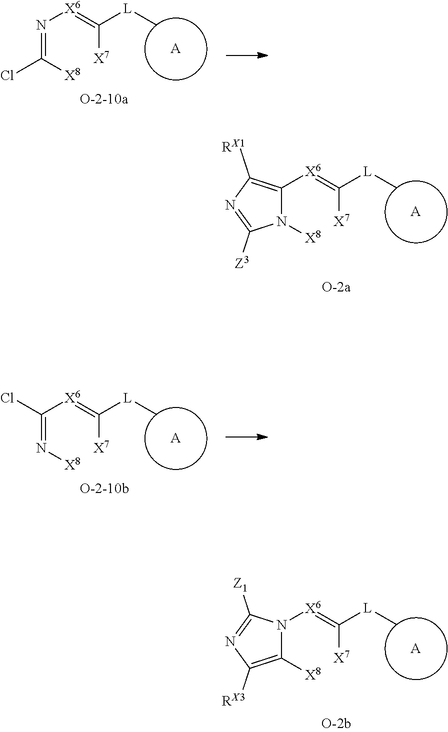

From O-2-10a/b:
By reaction with an appropriate isonitrile (based on the work described in Chem. Commun., 2016, 52, 2111-2113)

### Intermediate O-3

The intermediates O-3a/b can be obtained from multiple precursors including:

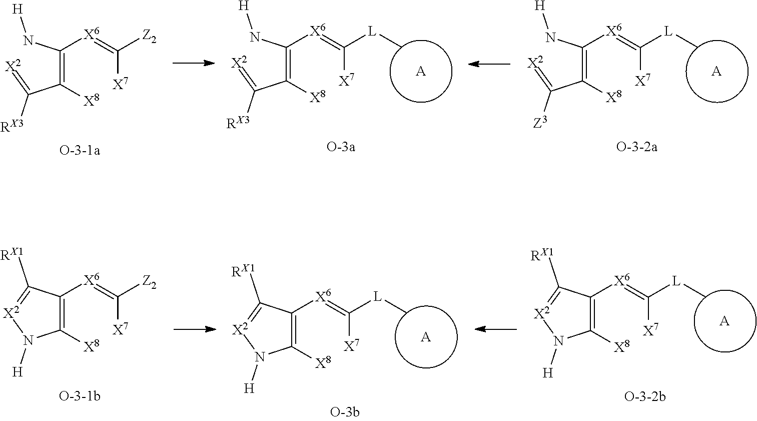

Through the above-mentioned general disconnection 1 from O-3-lalb.

Through the above-mentioned general disconnection 2 from O-3-2a/b.

More specific examples of intermediate O-3 can be obtained following the above-mentioned general disconnections 4 from precursors O-3-4-a/b, O-3-5-a/b, O-3-8, or O-3-9:

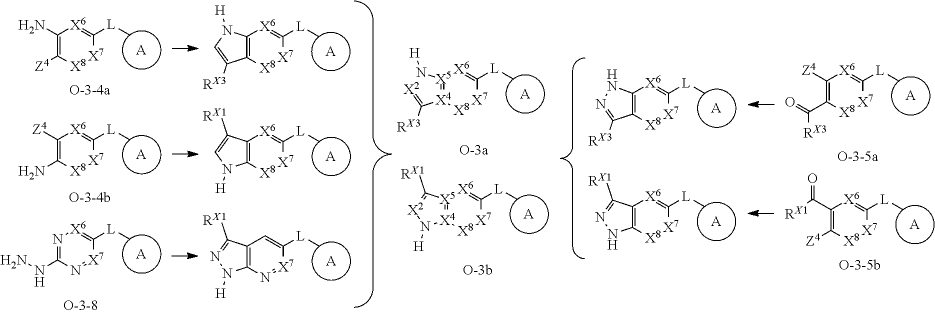

Precursors:

Precursors of O-1-X, O-2-X, O3-X, O-2a/b, O-4a/b, O-5a/b, O-6a/b, O-7a/b, O-8 or O-9 can be obtained through various synthetic pathways either precisely described in the corresponding reference from which their conversion into the corresponding O-1-X, O-2-X, O3-X, O-2a/b, O-4a/b, O-5a/b, O-6a/b, O-7a/b, O-8 or O-9 intermediate is described, or through the appropriate method mentioned in the general disconnections described above, or with a method known to the person skilled in the art.

The following definitions apply throughout the present specification and the claims, unless specifically indicated otherwise.

The term “hydrocarbon group” refers to a group consisting of carbon atoms and hydrogen atoms.

The term “alicyclic” is used in connection with cyclic groups and denotes that the corresponding cyclic group is non-aromatic.

As used herein, the term “alkyl” refers to a monovalent saturated acyclic (i.e., non-cyclic) hydrocarbon group which may be linear or branched. Accordingly, an “alkyl” group does not comprise any carbon-to-carbon double bond or any carbon-to-carbon triple bond. A “C₁₋₅ alkyl” denotes an alkyl group having 1 to 5 carbon atoms. Preferred exemplary alkyl groups are methyl, ethyl, propyl (e.g., n-propyl or isopropyl), or butyl (e.g., n-butyl, isobutyl, sec-butyl, or tert-butyl). Unless defined otherwise, the term “alkyl” preferably refers to C₁₋₄ alkyl, more preferably to methyl or ethyl, and even more preferably to methyl.

As used herein, the term “alkenyl” refers to a monovalent unsaturated acyclic hydrocarbon group which may be linear or branched and comprises one or more (e.g., one or two) carbon-to-carbon double bonds while it does not comprise any carbon-to-carbon triple bond. The term “C₂₋₅ alkenyl” denotes an alkenyl group having 2 to 5 carbon atoms.

Preferred exemplary alkenyl groups are ethenyl, propenyl (e.g., prop-1-en-1-yl, prop-1-en-2-yl, or prop-2-en-1-yl), butenyl, butadienyl (e.g., buta-1,3-dien-1-yl or buta-1,3-dien-2-yl), pentenyl, or pentadienyl (e.g., isoprenyl). Unless defined otherwise, the term “alkenyl” preferably refers to C₂₋₄ alkenyl.

As used herein, the term “alkynyl” refers to a monovalent unsaturated acyclic hydrocarbon group which may be linear or branched and comprises one or more (e.g., one or two) carbon-to-carbon triple bonds and optionally one or more (e.g., one or two) carbon-to-carbon double bonds. The term “C₂₋₅ alkynyl” denotes an alkynyl group having 2 to 5 carbon atoms. Preferred exemplary alkynyl groups are ethynyl, propynyl (e.g., propargyl), or butynyl. Unless defined otherwise, the term “alkynyl” preferably refers to C₂₋₄ alkynyl.

As used herein, the term “alkylene” refers to an alkanediyl group, i.e. a divalent saturated acyclic hydrocarbon group which may be linear or branched. A “C₁₋₅ alkylene” denotes an alkylene group having 1 to 5 carbon atoms, and the term “C₀₋₃ alkylene” indicates that a covalent bond (corresponding to the option “C₀ alkylene”) or a C₁₋₃ alkylene is present. Preferred exemplary alkylene groups are methylene (—CH₂—), ethylene (e.g., —CH₂—CH₂— or —CH(—CH₃)—), propylene (e.g., —CH₂—CH₂—CH₂—, —CH(—CH₂—CH₃)—, —CH₂—CH(—CH₃)—, or —CH(—CH₃)—CH₂—), or butylene (e.g., —CH₂—CH₂—CH₂—CH₂—). Unless defined otherwise, the term “alkylene” preferably refers to C₁₋₄ alkylene (including, in particular, linear C₁₋₄ alkylene), more preferably to methylene or ethylene, and even more preferably to methylene.

As used herein, the term “alkenylene” refers to an alkenediyl group, i.e. a divalent unsaturated acyclic hydrocarbon group which may be linear or branched and comprises one or more (e.g., one or two) carbon-to-carbon double bonds while it does not comprise any carbon-to-carbon triple bond. A “C₂₋₅ alkenylene” denotes an alkenylene group having 2 to 5 carbon atoms. Unless defined otherwise, the term “alkenylene” preferably refers to C₂₋₄ alkenylene (including, in particular, linear C₂₋₄ alkenylene).

As used herein, the term “alkynylene” refers to an alkynediyl group, i.e. a divalent unsaturated acyclic hydrocarbon group which may be linear or branched and comprises one or more (e.g., one or two) carbon-to-carbon triple bonds and optionally one or more (e.g., one or two) carbon-to-carbon double bonds. A “C₂₋₅ alkynylene” denotes an alkynylene group having 2 to 5 carbon atoms. Unless defined otherwise, the term “alkynylene” preferably refers to C₂₋₄ alkynylene (including, in particular, linear C₂₋₄ alkynylene).

As used herein, the term “carbocyclyl” refers to a hydrocarbon ring group, including monocyclic rings as well as bridged ring, spiro ring and/or fused ring systems (which may be composed, e.g., of two or three rings), wherein said ring group may be saturated, partially unsaturated (i.e., unsaturated but not aromatic) or aromatic. Unless defined otherwise, “carbocyclyl” preferably refers to aryl, cycloalkyl or cycloalkenyl.

As used herein, the term “heterocyclyl” refers to a ring group, including monocyclic rings as well as bridged ring, spiro ring and/or fused ring systems (which may be composed, e.g., of two or three rings), wherein said ring group comprises one or more (such as, e.g., one, two, three, or four) ring heteroatoms independently selected from 0, S and N, and the remaining ring atoms are carbon atoms, wherein one or more S ring atoms (if present) and/or one or more N ring atoms (if present) may optionally be oxidized, wherein one or more carbon ring atoms may optionally be oxidized (i.e., to form an oxo group), and further wherein said ring group may be saturated, partially unsaturated (i.e., unsaturated but not aromatic) or aromatic. For example, each heteroatom-containing ring comprised in said ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. Unless defined otherwise, “heterocyclyl” preferably refers to heteroaryl, heterocycloalkyl or heterocycloalkenyl.

As used herein, the term “aryl” refers to an aromatic hydrocarbon ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic). If the aryl is a bridged and/or fused ring system which contains, besides one or more aromatic rings, at least one non-aromatic ring (e.g., a saturated ring or an unsaturated alicyclic ring), then one or more carbon ring atoms in each non-aromatic ring may optionally be oxidized (i.e., to form an oxo group). “Aryl” may, e.g., refer to phenyl, naphthyl, dialinyl (i.e., 1,2-dihydronaphthyl), tetralinyl (i.e., 1,2,3,4-tetrahydronaphthyl), indanyl, indenyl (e.g., 1H-indenyl), anthracenyl, phenanthrenyl, 9H-fluorenyl, or azulenyl. Unless defined otherwise, an “aryl” preferably has 6 to 14 ring atoms, more preferably 6 to 10 ring atoms, even more preferably refers to phenyl or naphthyl, and most preferably refers to phenyl.

As used herein, the term “heteroaryl” refers to an aromatic ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic), wherein said aromatic ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). For example, each heteroatom-containing ring comprised in said aromatic ring group may contain one or two 0 atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heteroaryl” may, e.g., refer to thienyl (i.e., thiophenyl), benzo[b]thienyl, naphtho[2,3-b]thienyl, thianthrenyl, furyl (i.e., furanyl), benzofuranyl, isobenzofuranyl, chromanyl, chromenyl (e.g., 2H-1-benzopyranyl or 4H-1-benzopyranyl), isochromenyl (e.g., 1H-2-benzopyranyl), chromonyl, xanthenyl, phenoxathiinyl, pyrrolyl (e.g., 1H-pyrrolyl), imidazolyl, pyrazolyl, pyridyl (i.e., pyridinyl; e.g., 2-pyridyl, 3-pyridyl, or 4-pyridyl), pyrazinyl, pyrimidinyl, pyridazinyl, indolyl (e.g., 1H-indolyl), isoindolyl, indazolyl, indolizinyl, purinyl, quinolyl, isoquinolyl, phthalazinyl, naphthyridinyl, quinoxalinyl, cinnolinyl, pteridinyl, carbazolyl, β-carbolinyl, phenanthridinyl, acridinyl, perimidinyl, phenanthrolinyl (e.g., [1,10]phenanthrolinyl, [1,7]phenanthrolinyl, or [4,7]phenanthrolinyl), phenazinyl, thiazolyl, isothiazolyl, phenothiazinyl, oxazolyl, isoxazolyl, oxadiazolyl (e.g., 1,2,4-oxadiazolyl, 1,2,5-oxadiazolyl (i.e., furazanyl), or 1,3,4-oxadiazolyl), thiadiazolyl (e.g., 1,2,4-thiadiazolyl, 1,2,5-thiadiazolyl, or 1,3,4-thiadiazolyl), phenoxazinyl, pyrazolo[1,5-a]pyrimidinyl (e.g., pyrazolo[1,5-a]pyrimidin-3-yl), 1,2-benzoisoxazol-3-yl, benzothiazolyl, benzothiadiazolyl, benzoxazolyl, benzisoxazolyl, benzimidazolyl, benzo[b]thiophenyl (i.e., benzothienyl), triazolyl (e.g., 1H-1,2,3-triazolyl, 2H-1,2,3-triazolyl, 1H-1,2,4-triazolyl, or 4H-1,2,4-triazolyl), benzotriazolyl, 1H-tetrazolyl, 2H-tetrazolyl, triazinyl (e.g., 1,2,3-triazinyl, 1,2,4-triazinyl, or 1,3,5-triazinyl), furo[2,3-c]pyridinyl, dihydrofuropyridinyl (e.g., 2,3-dihydrofuro[2,3-c]pyridinyl or 1,3-dihydrofuro[3,4-c]pyridinyl), imidazopyridinyl (e.g., imidazo[1,2-a]pyridinyl or imidazo[3,2-a]pyridinyl), quinazolinyl, thienopyridinyl, tetrahydrothienopyridinyl (e.g., 4,5,6,7-tetrahydrothieno[3,2-c]pyridinyl), dibenzofuranyl, 1,3-benzodioxolyl, benzodioxanyl (e.g., 1,3-benzodioxanyl or 1,4-benzodioxanyl), or coumarinyl. Unless defined otherwise, the term “heteroaryl” preferably refers to a 5 to 14 membered (more preferably 5 to 10 membered) monocyclic ring or fused ring system comprising one or more (e.g., one, two, three or four) ring heteroatoms independently selected from 0, S and N, wherein one or more S ring atoms (if present) and/or one or more N ring atoms (if present) are optionally oxidized, and wherein one or more carbon ring atoms are optionally oxidized; even more preferably, a “heteroaryl” refers to a 5 or 6 membered monocyclic ring comprising one or more (e.g., one, two or three) ring heteroatoms independently selected from O, S and N, wherein one or more S ring atoms (if present) and/or one or more N ring atoms (if present) are optionally oxidized, and wherein one or more carbon ring atoms are optionally oxidized. Moreover, unless defined otherwise, particularly preferred examples of a “heteroaryl” include pyridinyl (e.g., 2-pyridyl, 3-pyridyl, or 4-pyridyl), imidazolyl, thiazolyl, 1H-tetrazolyl, 2H-tetrazolyl, thienyl (i.e., thiophenyl), or pyrimidinyl.

As used herein, the term “cycloalkyl” refers to a saturated hydrocarbon ring group, including monocyclic rings as well as bridged ring, spiro ring and/or fused ring systems (which may be composed, e.g., of two or three rings; such as, e.g., a fused ring system composed of two or three fused rings). “Cycloalkyl” may, e.g., refer to cyclopropyl, cyclobutyl, cyclopentyl, cyclohexyl, cycloheptyl, decalinyl (i.e., decahydronaphthyl), or adamantyl. Unless defined otherwise, “cycloalkyl” preferably refers to a C₃₋₁₁ cycloalkyl, and more preferably refers to a C₃₋₇ cycloalkyl. A particularly preferred “cycloalkyl” is a monocyclic saturated hydrocarbon ring having 3 to 7 ring members. Moreover, unless defined otherwise, particularly preferred examples of a “cycloalkyl” include cyclohexyl or cyclopropyl, particularly cyclohexyl.

As used herein, the term “heterocycloalkyl” refers to a saturated ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). For example, each heteroatom-containing ring comprised in said saturated ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heterocycloalkyl” may, e.g., refer to aziridinyl, azetidinyl, pyrrolidinyl, imidazolidinyl, pyrazolidinyl, piperidinyl, piperazinyl, azepanyl, diazepanyl (e.g., 1,4-diazepanyl), oxazolidinyl, isoxazolidinyl, thiazolidinyl, isothiazolidinyl, morpholinyl (e.g., morpholin-4-yl), thiomorpholinyl (e.g., thiomorpholin-4-yl), oxazepanyl, oxiranyl, oxetanyl, tetrahydrofuranyl, 1,3-dioxolanyl, tetrahydropyranyl, 1,4-dioxanyl, oxepanyl, thiiranyl, thietanyl, tetrahydrothiophenyl (i.e., thiolanyl), 1,3-dithiolanyl, thianyl, thiepanyl, decahydroquinolinyl, decahydroisoquinolinyl, or 2-oxa-5-aza-bicyclo[2.2.1]hept-5-yl. Unless defined otherwise, “heterocycloalkyl” preferably refers to a 3 to 11 membered saturated ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, and wherein one or more carbon ring atoms are optionally oxidized; more preferably, “heterocycloalkyl” refers to a 5 to 7 membered saturated monocyclic ring group containing one or more (e.g., one, two, or three) ring heteroatoms independently selected from O, S and N, wherein one or more S ring atoms (if present) and/or one or more N ring atoms (if present) are optionally oxidized, and wherein one or more carbon ring atoms are optionally oxidized. Moreover, unless defined otherwise, particularly preferred examples of a “heterocycloalkyl” include tetrahydropyranyl, piperidinyl, piperazinyl, morpholinyl, pyrrolidinyl, or tetrahydrofuranyl.

As used herein, the term “cycloalkenyl” refers to an unsaturated alicyclic (non-aromatic) hydrocarbon ring group, including monocyclic rings as well as bridged ring, spiro ring and/or fused ring systems (which may be composed, e.g., of two or three rings; such as, e.g., a fused ring system composed of two or three fused rings), wherein said hydrocarbon ring group comprises one or more (e.g., one or two) carbon-to-carbon double bonds and does not comprise any carbon-to-carbon triple bond. “Cycloalkenyl” may, e.g., refer to cyclopropenyl, cyclobutenyl, cyclopentenyl, cyclohexenyl, cyclohexadienyl, cycloheptenyl, or cycloheptadienyl. Unless defined otherwise, “cycloalkenyl” preferably refers to a C₃₋₁₁ cycloalkenyl, and more preferably refers to a C₃₋₇ cycloalkenyl. A particularly preferred “cycloalkenyl” is a monocyclic unsaturated alicyclic hydrocarbon ring having 3 to 7 ring members and containing one or more (e.g., one or two; preferably one) carbon-to-carbon double bonds.

As used herein, the term “heterocycloalkenyl” refers to an unsaturated alicyclic (non-aromatic) ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from 0, S and N, and the remaining ring atoms are carbon atoms, wherein one or more S ring atoms (if present) and/or one or more N ring atoms (if present) may optionally be oxidized, wherein one or more carbon ring atoms may optionally be oxidized (i.e., to form an oxo group), and further wherein said ring group comprises at least one double bond between adjacent ring atoms and does not comprise any triple bond between adjacent ring atoms. For example, each heteroatom-containing ring comprised in said unsaturated alicyclic ring group may contain one or two 0 atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heterocycloalkenyl” may, e.g., refer to imidazolinyl (e.g., 2-imidazolinyl (i.e., 4,5-dihydro-1H-imidazolyl), 3-imidazolinyl, or 4-imidazolinyl), tetrahydropyridinyl (e.g., 1,2,3,6-tetrahydropyridinyl), dihydropyridinyl (e.g., 1,2-dihydropyridinyl or 2,3-dihydropyridinyl), pyranyl (e.g., 2H-pyranyl or 4H-pyranyl), thiopyranyl (e.g., 2H-thiopyranyl or 4H-thiopyranyl), dihydropyranyl, dihydrofuranyl, dihydropyrazolyl, dihydropyrazinyl, dihydroisoindolyl, octahydroquinolinyl (e.g., 1,2,3,4,4a,5,6,7-octahydroquinolinyl), or octahydroisoquinolinyl (e.g., 1,2,3,4,5,6,7,8-octahydroisoquinolinyl). Unless defined otherwise, “heterocycloalkenyl” preferably refers to a 3 to 11 membered unsaturated alicyclic ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, wherein one or more carbon ring atoms are optionally oxidized, and wherein said ring group comprises at least one double bond between adjacent ring atoms and does not comprise any triple bond between adjacent ring atoms; more preferably, “heterocycloalkenyl” refers to a 5 to 7 membered monocyclic unsaturated non-aromatic ring group containing one or more (e.g., one, two, or three) ring heteroatoms independently selected from O, S and N, wherein one or more S ring atoms (if present) and/or one or more N ring atoms (if present) are optionally oxidized, wherein one or more carbon ring atoms are optionally oxidized, and wherein said ring group comprises at least one double bond between adjacent ring atoms and does not comprise any triple bond between adjacent ring atoms.

As used herein, the term “halogen” refers to fluoro (—F), chloro (—Cl), bromo (—Br), or iodo (—I).

As used herein, the term “haloalkyl” refers to an alkyl group substituted with one or more (preferably 1 to 6, more preferably 1 to 3) halogen atoms which are selected independently from fluoro, chloro, bromo and iodo, and are preferably all fluoro atoms. It will be understood that the maximum number of halogen atoms is limited by the number of available attachment sites and, thus, depends on the number of carbon atoms comprised in the alkyl moiety of the haloalkyl group. “Haloalkyl” may, e.g., refer to —CF₃, —CHF₂, —CH₂F, —CF₂—CH₃, —CH₂—CF₃, —CH₂—CHF₂, —CH₂—CF₂—CH₃, —CH₂—CF₂—CF₃, or —CH(CF₃)₂. A particularly preferred “haloalkyl” group is —CF₃.

The terms “bond” and “covalent bond” are used herein synonymously, unless explicitly indicated otherwise or contradicted by context.

As used herein, the terms “optional”, “optionally” and “may” denote that the indicated feature may be present but can also be absent. Whenever the term “optional”, “optionally” or “may” is used, the present invention specifically relates to both possibilities, i.e., that the corresponding feature is present or, alternatively, that the corresponding feature is absent. For example, the expression “X is optionally substituted with Y” (or “X may be substituted with Y”) means that X is either substituted with Y or is unsubstituted. Likewise, if a component of a composition is indicated to be “optional”, the invention specifically relates to both possibilities, i.e., that the corresponding component is present (contained in the composition) or that the corresponding component is absent from the composition.

Various groups are referred to as being “optionally substituted” in this specification. Generally, these groups may carry one or more substituents, such as, e.g., one, two, three or four substituents. It will be understood that the maximum number of substituents is limited by the number of attachment sites available on the substituted moiety. Unless defined otherwise, the “optionally substituted” groups referred to in this specification carry preferably not more than two substituents and may, in particular, carry only one substituent. Moreover, unless defined otherwise, it is preferred that the optional substituents are absent, i.e. that the corresponding groups are unsubstituted.

A skilled person will appreciate that the substituent groups comprised in the compounds of the present invention may be attached to the remainder of the respective compound via a number of different positions of the corresponding specific substituent group. Unless defined otherwise, the preferred attachment positions for the various specific substituent groups are as illustrated in the examples.

As used herein, unless explicitly indicated otherwise or contradicted by context, the terms “a”, “an” and “the” are used interchangeably with “one or more” and “at least one”. Thus, for example, a composition comprising “a” compound of formula (I) can be interpreted as referring to a composition comprising “one or more” compounds of formula (I).

It is to be understood that wherever numerical ranges are provided/disclosed herein, all values and subranges encompassed by the respective numerical range are meant to be encompassed within the scope of the invention. Accordingly, the present invention specifically and individually relates to each value that falls within a numerical range disclosed herein, as well as each subrange encompassed by a numerical range disclosed herein.

As used herein, the term “about” preferably refers to ±10% of the indicated numerical value, more preferably to ±5% of the indicated numerical value, and in particular to the exact numerical value indicated. If the term “about” is used in connection with the endpoints of a range, it preferably refers to the range from the lower endpoint −10% of its indicated numerical value to the upper endpoint+10% of its indicated numerical value, more preferably to the range from of the lower endpoint −5% to the upper endpoint+5%, and even more preferably to the range defined by the exact numerical values of the lower endpoint and the upper endpoint.

As used herein, the term “comprising” (or “comprise”, “comprises”, “contain”, “contains”, or “containing”), unless explicitly indicated otherwise or contradicted by context, has the meaning of “containing, inter alia”, i.e., “containing, among further optional elements, . . . ”. In addition thereto, this term also includes the narrower meanings of “consisting essentially of” and “consisting of”. For example, the term “A comprising B and C” has the meaning of “A containing, inter alia, B and C”, wherein A may contain further optional elements (e.g., “A containing B, C and D” would also be encompassed), but this term also includes the meaning of “A consisting essentially of B and C” and the meaning of “A consisting of B and C” (i.e., no other components than B and C are comprised in A).

The scope of the invention embraces all pharmaceutically acceptable salt forms of the compounds of formula (I) which may be formed, e.g., by protonation of an atom carrying an electron lone pair which is susceptible to protonation, such as an amino group, with an inorganic or organic acid, or as a salt of an acid group (such as a carboxylic acid group) with a physiologically acceptable cation. Exemplary base addition salts comprise, for example: alkali metal salts such as sodium or potassium salts; alkaline earth metal salts such as calcium or magnesium salts; zinc salts; ammonium salts; aliphatic amine salts such as trimethylamine, triethylamine, dicyclohexylamine, ethanolamine, diethanolamine, triethanolamine, procaine salts, meglumine salts, ethylenediamine salts, or choline salts; aralkyl amine salts such as N,N-dibenzylethylenediamine salts, benzathine salts, benethamine salts; heterocyclic aromatic amine salts such as pyridine salts, picoline salts, quinoline salts or isoquinoline salts; quaternary ammonium salts such as tetramethylammonium salts, tetraethylammonium salts, benzyltrimethylammonium salts, benzyltriethylammonium salts, benzyltributylammonium salts, methyltrioctylammonium salts or tetrabutylammonium salts; and basic amino acid salts such as arginine salts, lysine salts, or histidine salts. Exemplary acid addition salts comprise, for example: mineral acid salts such as hydrochloride, hydrobromide, hydroiodide, sulfate salts (such as, e.g., sulfate or hydrogensulfate salts), nitrate salts, phosphate salts (such as, e.g., phosphate, hydrogenphosphate, or dihydrogenphosphate salts), carbonate salts, hydrogencarbonate salts, perchlorate salts, borate salts, or thiocyanate salts; organic acid salts such as acetate, propionate, butyrate, pentanoate, hexanoate, heptanoate, octanoate, cyclopentanepropionate, decanoate, undecanoate, oleate, stearate, lactate, maleate, oxalate, fumarate, tartrate, malate, citrate, succinate, adipate, gluconate, glycolate, nicotinate, benzoate, salicylate, ascorbate, pamoate (embonate), camphorate, glucoheptanoate, or pivalate salts; sulfonate salts such as methanesulfonate (mesylate), ethanesulfonate (esylate), 2-hydroxyethanesulfonate (isethionate), benzenesulfonate (besylate), p-toluenesulfonate (tosylate), 2-naphthalenesulfonate (napsylate), 3-phenylsulfonate, or camphorsulfonate salts; glycerophosphate salts; and acidic amino acid salts such as aspartate or glutamate salts. Further pharmaceutically acceptable salts are described in the literature, e.g., in Stahl P H & Wermuth C G (eds.), “Handbook of Pharmaceutical Salts: Properties, Selection, and Use”, Wiley-VCH, 2002 and in the references cited therein. Preferred pharmaceutically acceptable salts of the compounds of formula (I) include a hydrochloride salt, a hydrobromide salt, a mesylate salt, a sulfate salt, a tartrate salt, a fumarate salt, an acetate salt, a citrate salt, and a phosphate salt. A particularly preferred pharmaceutically acceptable salt of the compound of formula (I) is a hydrochloride salt. Accordingly, it is preferred that the compound of formula (I), including any one of the specific compounds of formula (I) described herein, is in the form of a hydrochloride salt, a hydrobromide salt, a mesylate salt, a sulfate salt, a tartrate salt, a fumarate salt, an acetate salt, a citrate salt, or a phosphate salt, and it is particularly preferred that the compound of formula (I) is in the form of a hydrochloride salt.

The present invention also specifically relates to the compound of formula (I), including any one of the specific compounds of formula (I) described herein, in non-salt form.

Moreover, the scope of the invention embraces the compounds of formula (I) in any solvated form, including, e.g., solvates with water (i.e., as a hydrate) or solvates with organic solvents such as, e.g., methanol, ethanol, isopropanol, acetic acid, ethyl acetate, ethanolamine, DMSO, or acetonitrile. All physical forms, including any amorphous or crystalline forms (i.e., polymorphs), of the compounds of formula (I) are also encompassed within the scope of the invention. It is to be understood that such solvates and physical forms of pharmaceutically acceptable salts of the compounds of the formula (I) are likewise embraced by the invention.

Furthermore, the compounds of formula (I) may exist in the form of different isomers, in particular stereoisomers (including, e.g., geometric isomers (or cis/trans isomers), enantiomers and diastereomers) or tautomers (including, in particular, prototropic tautomers, such as keto/enol tautomers or thione/thiol tautomers). All such isomers of the compounds of formula (I) are contemplated as being part of the present invention, either in admixture or in pure or substantially pure form. As for stereoisomers, the invention embraces the isolated optical isomers of the compounds according to the invention as well as any mixtures thereof (including, in particular, racemic mixtures/racemates). The racemates can be resolved by physical methods, such as, e.g., fractional crystallization, separation or crystallization of diastereomeric derivatives, or separation by chiral column chromatography. The individual optical isomers can also be obtained from the racemates via salt formation with an optically active acid followed by crystallization. The present invention further encompasses any tautomers of the compounds of formula (I). It will be understood that some compounds may exhibit tautomerism. In such cases, the formulae provided herein expressly depict only one of the possible tautomeric forms. The formulae and chemical names as provided herein are intended to encompass any tautomeric form of the corresponding compound and not to be limited merely to the specific tautomeric form depicted by the drawing or identified by the name of the compound.

The scope of the invention also embraces compounds of formula (I), in which one or more atoms are replaced by a specific isotope of the corresponding atom. For example, the invention encompasses compounds of formula (I), in which one or more hydrogen atoms (or, e.g., all hydrogen atoms) are replaced by deuterium atoms (i.e., ²H; also referred to as “D”). Accordingly, the invention also embraces compounds of formula (I) which are enriched in deuterium. Naturally occurring hydrogen is an isotopic mixture comprising about 99.98 mol-% hydrogen-1 (¹H) and about 0.0156 mol-% deuterium (²H or D). The content of deuterium in one or more hydrogen positions in the compounds of formula (I) can be increased using deuteration techniques known in the art. For example, a compound of formula (I) or a reactant or precursor to be used in the synthesis of the compound of formula (I) can be subjected to an H/D exchange reaction using, e.g., heavy water (D₂O). Further suitable deuteration techniques are described in: Atzrodt J et al., Bioorg Med Chem, 20(18), 5658-5667, 2012; William J S et al., Journal of Labelled Compounds and Radiopharmaceuticals, 53(11-12), 635-644, 2010; Modvig A et al., J Org Chem, 79, 5861-5868, 2014. The content of deuterium can be determined, e.g., using mass spectrometry or NMR spectroscopy. Unless specifically indicated otherwise, it is preferred that the compound of formula (I) is not enriched in deuterium. Accordingly, the presence of naturally occurring hydrogen atoms or ¹H hydrogen atoms in the compounds of formula (I) is preferred.

The present invention also embraces compounds of formula (I), in which one or more atoms are replaced by a positron-emitting isotope of the corresponding atom, such as, e.g., ¹⁸F, ¹¹C, ¹³N, ¹⁵O, ⁷⁶Br, ⁷⁷Br, ¹²⁰I and/or ¹²⁴I. Such compounds can be used as tracers, trackers or imaging probes in positron emission tomography (PET). The invention thus includes (i) compounds of formula (I), in which one or more fluorine atoms (or, e.g., all fluorine atoms) are replaced by ¹⁸F atoms, (ii) compounds of formula (I), in which one or more carbon atoms (or, e.g., all carbon atoms) are replaced by ¹¹C atoms, (iii) compounds of formula (I), in which one or more nitrogen atoms (or, e.g., all nitrogen atoms) are replaced by ¹³N atoms, (iv) compounds of formula (I), in which one or more oxygen atoms (or, e.g., all oxygen atoms) are replaced by ¹⁵O atoms, (v) compounds of formula (I), in which one or more bromine atoms (or, e.g., all bromine atoms) are replaced by ⁷⁶Br atoms, (vi) compounds of formula (I), in which one or more bromine atoms (or, e.g., all bromine atoms) are replaced by ⁷⁷Br atoms, (vii) compounds of formula (I), in which one or more iodine atoms (or, e.g., all iodine atoms) are replaced by ¹²⁰I atoms, and (viii) compounds of formula (I), in which one or more iodine atoms (or, e.g., all iodine atoms) are replaced by ¹²⁴I atoms. In general, it is preferred that none of the atoms in the compounds of formula (I) are replaced by specific isotopes.

The compounds provided herein may be administered as compounds per se or may be formulated as medicaments. The medicaments/pharmaceutical compositions may optionally comprise one or more pharmaceutically acceptable excipients, such as carriers, diluents, fillers, disintegrants, lubricating agents, binders, colorants, pigments, stabilizers, preservatives, antioxidants, and/or solubility enhancers.

The pharmaceutical compositions may comprise one or more solubility enhancers, such as, e.g., poly(ethylene glycol), including poly(ethylene glycol) having a molecular weight in the range of about 200 to about 5,000 Da (e.g., PEG 200, PEG 300, PEG 400, or PEG 600), ethylene glycol, propylene glycol, glycerol, a non-ionic surfactant, tyloxapol, polysorbate 80, macrogol-15-hydroxystearate (e.g., Kolliphor® HS 15, CAS 70142-34-6), a phospholipid, lecithin, dimyristoyl phosphatidylcholine, dipalmitoyl phosphatidylcholine, distearoyl phosphatidylcholine, a cyclodextrin, α-cyclodextrin, β-cyclodextrin, γ-cyclodextrin, hydroxyethyl-β-cyclodextrin, hydroxypropyl-β-cyclodextrin, hydroxyethyl-γ-cyclodextrin, hydroxypropyl-γ-cyclodextrin, dihydroxypropyl-β-cyclodextrin, sulfobutylether-β-cyclodextrin, sulfobutylether-γ-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, a carboxyalkyl thioether, hydroxypropyl methylcellulose, hydroxypropylcellulose, polyvinylpyrrolidone, a vinyl acetate copolymer, vinyl pyrrolidone, sodium lauryl sulfate, dioctyl sodium sulfosuccinate, or any combination thereof.

The pharmaceutical compositions may also comprise one or more preservatives, particularly one or more antimicrobial preservatives, such as, e.g., benzyl alcohol, chlorobutanol, 2-ethoxyethanol, m-cresol, chlorocresol (e.g., 2-chloro-3-methyl-phenol or 4-chloro-3-methyl-phenol), benzalkonium chloride, benzethonium chloride, benzoic acid (or a pharmaceutically acceptable salt thereof), sorbic acid (or a pharmaceutically acceptable salt thereof), chlorhexidine, thimerosal, or any combination thereof.

The pharmaceutical compositions can be formulated by techniques known to the person skilled in the art, such as the techniques published in “Remington: The Science and Practice of Pharmacy”, Pharmaceutical Press, 22^{nd} edition. The pharmaceutical compositions can be formulated as dosage forms for oral, parenteral, such as intramuscular, intravenous, subcutaneous, intradermal, intraarterial, intracardial, rectal, nasal, topical, aerosol or vaginal administration. Dosage forms for oral administration include coated and uncoated tablets, soft gelatin capsules, hard gelatin capsules, lozenges, troches, solutions, emulsions, suspensions, syrups, elixirs, powders and granules for reconstitution, dispersible powders and granules, medicated gums, chewing tablets and effervescent tablets. Dosage forms for parenteral administration include solutions, emulsions, suspensions, dispersions and powders and granules for reconstitution. Emulsions are a preferred dosage form for parenteral administration. Dosage forms for rectal and vaginal administration include suppositories and ovula. Dosage forms for nasal administration can be administered via inhalation and insufflation, for example by a metered inhaler. Dosage forms for topical administration include creams, gels, ointments, salves, patches and transdermal delivery systems.

The compounds of formula (I) or the pharmaceutically acceptable salts or solvates thereof, or the above described pharmaceutical compositions comprising any of the aforementioned entities, may be administered to a subject by any convenient route of administration, whether systemically/peripherally or at the site of desired action, including but not limited to one or more of: oral (e.g., as a tablet, capsule, or as an ingestible solution), topical (e.g., transdermal, intranasal, ocular, buccal, and sublingual), parenteral (e.g., using injection techniques or infusion techniques, and including, for example, by injection, e.g., subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, or intrasternal by, e.g., implant of a depot, for example, subcutaneously or intramuscularly), pulmonary (e.g., by inhalation or insufflation therapy using, e.g., an aerosol, e.g., through mouth or nose), gastrointestinal, intrauterine, intraocular, subcutaneous, ophthalmic (including intravitreal or intracameral), rectal, or vaginal administration.

If said compounds or pharmaceutical compositions are administered parenterally, then examples of such administration include one or more of: intravenously, intraarterially, intraperitoneally, intrathecally, intraventricularly, intraurethrally, intrasternally, intracardially, intracranially, intramuscularly or subcutaneously administering the compounds or pharmaceutical compositions, and/or by using infusion techniques. For parenteral administration, the compounds are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

Said compounds or pharmaceutical compositions can also be administered orally in the form of tablets, capsules, ovules, elixirs, solutions or suspensions, which may contain flavoring or coloring agents, for immediate-, delayed-, modified-, sustained-, pulsed- or controlled-release applications.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included. Solid compositions of a similar type may also be employed as fillers in gelatin capsules. Preferred excipients in this regard include lactose, starch, a cellulose, or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

For oral administration, the compounds or pharmaceutical compositions are preferably administered by oral ingestion, particularly by swallowing. The compounds or pharmaceutical compositions can thus be administered to pass through the mouth into the gastrointestinal tract, which can also be referred to as “oral-gastrointestinal” administration.

Alternatively, said compounds or pharmaceutical compositions can be administered in the form of a suppository or pessary, or may be applied topically in the form of a gel, hydrogel, lotion, solution, cream, ointment or dusting powder. The compounds of the present invention may also be dermally or transdermally administered, for example, by the use of a skin patch.

Said compounds or pharmaceutical compositions may also be administered by sustained release systems. Suitable examples of sustained-release compositions include semi-permeable polymer matrices in the form of shaped articles, e.g., films, or microcapsules. Sustained-release matrices include, e.g., polylactides, copolymers of L-glutamic acid and gamma-ethyl-L-glutamate, poly(2-hydroxyethyl methacrylate), ethylene vinyl acetate, or poly-D-(−)-3-hydroxybutyric acid. Sustained-release pharmaceutical compositions also include liposomally entrapped compounds. The present invention thus also relates to liposomes containing a compound of the invention.

Said compounds or pharmaceutical compositions may also be administered by the pulmonary route, rectal routes, or the ocular route. For ophthalmic use, they can be formulated as micronized suspensions in isotonic, pH adjusted, sterile saline, or, preferably, as solutions in isotonic, pH adjusted, sterile saline, optionally in combination with a preservative such as a benzalkonium chloride. Alternatively, they may be formulated in an ointment such as petrolatum.

It is also envisaged to prepare dry powder formulations of the compounds of formula (I) for pulmonary administration, particularly inhalation. Such dry powders may be prepared by spray drying under conditions which result in a substantially amorphous glassy or a substantially crystalline bioactive powder. Accordingly, dry powders of the compounds of the present invention can be made according to an emulsification/spray drying process.

For topical application to the skin, said compounds or pharmaceutical compositions can be formulated as a suitable ointment containing the active compound suspended or dissolved in, for example, a mixture with one or more of the following: mineral oil, liquid petrolatum, white petrolatum, propylene glycol, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, 2-octyldodecanol, benzyl alcohol and water.

The present invention thus relates to the compounds or the pharmaceutical compositions provided herein, wherein the corresponding compound or pharmaceutical composition is to be administered by any one of: an oral route; topical route, including by transdermal, intranasal, ocular, buccal, or sublingual route; parenteral route using injection techniques or infusion techniques, including by subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, intrasternal, intraventricular, intraurethral, or intracranial route; pulmonary route, including by inhalation or insufflation therapy; gastrointestinal route; intrauterine route; intraocular route; subcutaneous route; ophthalmic route, including by intravitreal, or intracameral route; rectal route; or vaginal route. Preferred routes of administration are oral administration or parenteral administration. For each of the compounds or pharmaceutical compositions provided herein, it is particularly preferred that the respective compound or pharmaceutical composition is to be administered orally (particularly by oral ingestion).

Typically, a physician will determine the actual dosage which will be most suitable for an individual subject. The specific dose level and frequency of dosage for any particular individual subject may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual subject undergoing therapy. A proposed, yet non-limiting dose of the compounds according to the invention for oral administration to a human (of approximately 70 kg body weight) may be 0.05 to 2000 mg, preferably 0.1 mg to 1000 mg, of the active ingredient per unit dose. The unit dose may be administered, e.g., 1 to 3 times per day. The unit dose may also be administered 1 to 7 times per week, e.g., with not more than one administration per day. It will be appreciated that it may be necessary to make routine variations to the dosage depending on the age and weight of the patient/subject as well as the severity of the condition to be treated. The precise dose and also the route of administration will ultimately be at the discretion of the attendant physician or veterinarian.

The compound of formula (I) or the pharmaceutically acceptable salt or solvate thereof, or a pharmaceutical composition comprising any of the aforementioned entities, can be administered in monotherapy (e.g., without concomitantly administering any further therapeutic agents, or without concomitantly administering any further therapeutic agents against the same disease that is to be treated or prevented with the compound of formula (I)). However, the compound of formula (I) or the pharmaceutically acceptable salt or solvate thereof, or a pharmaceutical composition comprising any of the aforementioned entities, can also be administered in combination with one or more further therapeutic agents. If the compound of formula (I) is used in combination with a second therapeutic agent active against the same disease or condition, the dose of each compound may differ from that when the corresponding compound is used alone, in particular, a lower dose of each compound may be used. The combination of the compound of formula (I) with one or more further therapeutic agents may comprise the simultaneous/concomitant administration of the compound of formula (I) and the further therapeutic agent(s) (either in a single pharmaceutical formulation or in separate pharmaceutical formulations), or the sequential/separate administration of the compound of formula (I) and the further therapeutic agent(s). If administration is sequential, either the compound of formula (I) according to the invention or the one or more further therapeutic agents may be administered first. If administration is simultaneous, the one or more further therapeutic agents may be included in the same pharmaceutical formulation as the compound of formula (I), or they may be administered in two or more different (separate) pharmaceutical formulations.

For the treatment or prevention of cancer, the one or more further therapeutic agents to be administered in combination with a compound of the present invention are preferably anticancer drugs. The anticancer drug(s) to be administered in combination with a compound of formula (I) or a pharmaceutically acceptable salt or solvate thereof may, e.g., be selected from: a tumor angiogenesis inhibitor (e.g., a protease inhibitor, an epidermal growth factor receptor kinase inhibitor, or a vascular endothelial growth factor receptor kinase inhibitor); a cytotoxic drug (e.g., an antimetabolite, such as purine and pyrimidine analog antimetabolites); an antimitotic agent (e.g., a microtubule stabilizing drug or an antimitotic alkaloid); a platinum coordination complex; an anti-tumor antibiotic; an alkylating agent (e.g., a nitrogen mustard or a nitrosourea); an endocrine agent (e.g., an adrenocorticosteroid, an androgen, an anti-androgen, an estrogen, an anti-estrogen, an aromatase inhibitor, a gonadotropin-releasing hormone agonist, or a somatostatin analog); or a compound that targets an enzyme or receptor that is overexpressed and/or otherwise involved in a specific metabolic pathway that is deregulated (or misregulated) in the tumor cell (e.g., ATP and GTP phosphodiesterase inhibitors, histone deacetylase inhibitors, protein kinase inhibitors (such as serine, threonine and tyrosine kinase inhibitors, e.g., Abelson protein tyrosine kinase inhibitors) and the various growth factors, their receptors and corresponding kinase inhibitors (such as epidermal growth factor receptor kinase inhibitors, vascular endothelial growth factor receptor kinase inhibitors, fibroblast growth factor inhibitors, insulin-like growth factor receptor inhibitors and platelet-derived growth factor receptor kinase inhibitors)); methionine, aminopeptidase inhibitors, proteasome inhibitors, cyclooxygenase inhibitors (e.g., cyclooxygenase-1 or cyclooxygenase-2 inhibitors), topoisomerase inhibitors (e.g., topoisomerase I inhibitors or topoisomerase II inhibitors), poly ADP ribose polymerase inhibitors (PARP inhibitors), and epidermal growth factor receptor (EGFR) inhibitors/antagonists.

An alkylating agent which can be used as an anticancer drug in combination with a compound of the present invention may be, for example, a nitrogen mustard (such as cyclophosphamide, mechlorethamine (chlormethine), uramustine, melphalan, chlorambucil, ifosfamide, bendamustine, or trofosfamide), a nitrosourea (such as carmustine, streptozocin, fotemustine, lomustine, nimustine, prednimustine, ranimustine, or semustine), an alkyl sulfonate (such as busulfan, mannosulfan, or treosulfan), an aziridine (such as hexamethylmelamine (altretamine), triethylenemelamine, ThioTEPA (N,N′N′-triethylenethiophosphoramide), carboquone, or triaziquone), a hydrazine (such as procarbazine), a triazene (such as dacarbazine), or an imidazotetrazine (such as temozolomide).

A platinum coordination complex which can be used as an anticancer drug in combination with a compound of the present invention may be, for example, cisplatin, carboplatin, nedaplatin, oxaliplatin, satraplatin, or triplatin tetranitrate.

A cytotoxic drug which can be used as an anticancer drug in combination with a compound of the present invention may be, for example, an antimetabolite, including folic acid analogue antimetabolites (such as aminopterin, methotrexate, pemetrexed, or raltitrexed), purine analogue antimetabolites (such as cladribine, clofarabine, fludarabine, 6-mercaptopurine (including its prodrug form azathioprine), pentostatin, or 6-thioguanine), and pyrimidine analogue antimetabolites (such as cytarabine, decitabine, 5-fluorouracil (including its prodrug forms capecitabine and tegafur), floxuridine, gemcitabine, enocitabine, or sapacitabine).

An antimitotic agent which can be used as an anticancer drug in combination with a compound of the present invention may be, for example, a taxane (such as docetaxel, larotaxel, ortataxel, paclitaxel/taxol, tesetaxel, or nab-paclitaxel (e.g., Abraxane®)), a Vinca alkaloid (such as vinblastine, vincristine, vinflunine, vindesine, or vinorelbine), an epothilone (such as epothilone A, epothilone B, epothilone C, epothilone D, epothilone E, or epothilone F) or an epothilone B analogue (such as ixabepilone/azaepothilone B).

An anti-tumor antibiotic which can be used as an anticancer drug in combination with a compound of the present invention may be, for example, an anthracycline (such as aclarubicin, daunorubicin, doxorubicin, epirubicin, idarubicin, amrubicin, pirarubicin, valrubicin, or zorubicin), an anthracenedione (such as mitoxantrone, or pixantrone) or an anti-tumor antibiotic isolated from Streptomyces (such as actinomycin (including actinomycin D), bleomycin, mitomycin (including mitomycin C), or plicamycin).

A tyrosine kinase inhibitor which can be used as an anticancer drug in combination with a compound of the present invention may be, for example, axitinib, bosutinib, cediranib, dasatinib, erlotinib, gefitinib, imatinib, lapatinib, lestaurtinib, nilotinib, semaxanib, sorafenib, sunitinib, axitinib, nintedanib, ponatinib, vandetanib, or vemurafenib.

A topoisomerase inhibitor which can be used as an anticancer drug in combination with a compound of the present invention may be, for example, a topoisomerase I inhibitor (such as irinotecan, topotecan, camptothecin, belotecan, rubitecan, or lamellarin D) or a topoisomerase II inhibitor (such as amsacrine, etoposide, etoposide phosphate, teniposide, or doxorubicin).

A PARP inhibitor which can be used as an anticancer drug in combination with a compound of the present invention may be, for example, niraparib, olaparib, rucaparib, talazoparib, veliparib, pamiparib (BGB-290), BMN-673, CEP 9722, MK 4827, E7016, or 3-aminobenzamide.

An EGFR inhibitor/antagonist which can be used as an anticancer drug in combination with a compound of the present invention may be, for example, gefitinib, erlotinib, lapatinib, afatinib, neratinib, osimertinib, brigatinib, dacomitinib, vandetanib, pelitinib, canertinib, icotinib, poziotinib, ABT-414, AV-412, PD 153035, PKI-166, BMS-690514, CUDC-101, AP26113, XL647, cetuximab, panitumumab, zalutumumab, nimotuzumab, or matuzumab.

Further anticancer drugs may also be used in combination with a compound of the present invention. The anticancer drugs may comprise biological or chemical molecules, like TNF-related apoptosis-inducing ligand (TRAIL), tamoxifen, amsacrine, bexarotene, estramustine, irofulven, trabectedin, cetuximab, panitumumab, tositumomab, alemtuzumab, bevacizumab, edrecolomab, gemtuzumab, alvocidib, seliciclib, aminolevulinic acid, methyl aminolevulinate, efaproxiral, porfimer sodium, talaporfin, temoporfin, verteporfin, alitretinoin, tretinoin, anagrelide, arsenic trioxide, atrasentan, bortezomib, carmofur, celecoxib, demecolcine, elesclomol, elsamitrucin, etoglucid, lonidamine, lucanthone, masoprocol, mitobronitol, mitoguazone, mitotane, oblimersen, omacetaxine, sitimagene, ceradenovec, tegafur, testolactone, tiazofurine, tipifarnib, vorinostat, iniparib, or copanlisib.

Also biological drugs, like antibodies, antibody fragments, antibody constructs (for example, single-chain constructs), and/or modified antibodies (like CDR-grafted antibodies, humanized antibodies, “fully human” antibodies, etc.) directed against cancer or tumor markers/factors/cytokines involved in proliferative diseases can be employed in cotherapy approaches with the compounds of the invention. Examples of such biological molecules are anti-HER2 antibodies (e.g. trastuzumab, Herceptin®), anti-CD20 antibodies (e.g. Rituximab, Rituxan®, MabThera®, Reditux®), anti-CD19/CD3 constructs, and anti-TNF antibodies (see, e.g., Taylor P C, Curr Opin Pharmacol, 2003, 3(3):323-328). An anticancer drug which can be used in combination with a compound of the present invention may, in particular, be an immunooncology therapeutic (such as an antibody (e.g., a monoclonal antibody or a polyclonal antibody), an antibody fragment, an antibody construct (e.g., a single-chain construct), or a modified antibody (e.g., a CDR-grafted antibody, a humanized antibody, or a “fully human” antibody) targeting any one of CTLA-4, PD-1, PD-L1, TIGIT, TIM3, LAG3, OX⁴⁰, CSF1R, IDO, or CD40. Such immunooncology therapeutics include, e.g., an anti-CTLA-4 antibody (e.g., ipilimumab or tremelimumab), an anti-PD-1 antibody (e.g., nivolumab (BMS-936558), pembrolizumab (MK-3475), pidilizumab (CT-011), cemiplimab, dostarlimab, spartalizumab, camrelizumab, sintilimab, tislelizumab, toripalimab, zimberelimab, AMP-224, AMP-514 (or MEDI0680), JTX-4014, INCMGA00012 (or MGA012), or APE02058), an anti-PD-L1 antibody (e.g., atezolizumab, avelumab, durvalumab, KN035, CK-301, BMS-936559, MEDI4736, MPDL3280A (RG7446), MDX-1105, MEDI6469, or bintrafusp alfa), an anti-TIGIT antibody (e.g., tiragolumab, vibostolimab, domvanalimab, etigilimab, BMS-986207, EOS-448, COM902, ASP8374, SEA-TGT, BGB-A1217, IBI-939, or M6223), an anti-TIM3 antibody, an anti-LAG3 antibody (e.g., relatlimab (or BMS-986016), ieramilimab (or LAG525), encelimab (or TSR-033), tebotelimab (or MGD013), REGN3767 (or R3767), FS118, IMP701, or IMP731), an anti-OX⁴⁰ antibody (e.g., MEDI0562), an anti-CSF1R antibody (e.g., IMC—CS4 or RG7155), an anti-IDO antibody, or an anti-CD40 antibody (e.g., CP-870,893 or Chi Lob 7/4). Further immunooncology therapeutics are known in the art and are described, e.g., in: Kyi C et al., FEBS Lett, 2014, 588(2):368-76; Intlekofer A M et al., J Leukoc Biol, 2013, 94(1):25-39; Callahan M K et al., J Leukoc Biol, 2013, 94(1):41-53; Ngiow S F et al., Cancer Res, 2011, 71(21):6567-71; and Blattman J N et al., Science, 2004, 305(5681):200-5.

In particular, a compound of formula (I) or a pharmaceutically acceptable salt or solvate thereof, or a pharmaceutical composition comprising any of the aforementioned entities, may be administered in combination with an immune checkpoint inhibitor, preferably an antibody (or an antigen-binding fragment thereof, or an antibody construct) directed against CTLA-4, PD-1, PD-L1, TIGIT, or LAG3. Corresponding preferred examples include, but are not limited to, any one of the anti-CTLA-4 antibodies ipilimumab or tremelimumab, any one of the anti-PD-1 antibodies nivolumab, pembrolizumab, pidilizumab, cemiplimab, dostarlimab, spartalizumab, camrelizumab, sintilimab, tislelizumab, toripalimab, zimberelimab, AMP-224, AMP-514, JTX-4014, INCMGA00012, or APE02058, any one of the anti-PD-L1 antibodies atezolizumab, avelumab, durvalumab, KN035, CK-301, BMS-936559, MED14736, MPDL3280A, MDX-1105, MED16469 or bintrafusp alfa, any one of the anti-TIGIT antibodies tiragolumab, vibostolimab, domvanalimab, etigilimab, BMS-986207, EOS-448, COM902, ASP8374, SEA-TGT, BGB-A1217, IBI-939 or M6223, and/or any one of the anti-LAG3 antibodies relatlimab, ieramilimab, encelimab, tebotelimab, REGN3767, FS118, IMP701, or IMP731. The present invention thus relates to a compound of formula (I) or a pharmaceutically acceptable salt or solvates thereof, or a pharmaceutical composition comprising any of the aforementioned entities optionally in combination with a pharmaceutically acceptable excipient, for use in the treatment or prevention of cancer, wherein the compound or the pharmaceutical composition is to be administered in combination with one or more immune checkpoint inhibitors, wherein said one or more immune checkpoint inhibitors are preferably selected from anti-CTLA-4 antibodies, anti-PD-1 antibodies, anti-PD-L1 antibodies, anti-TIGIT antibodies, and/or anti-LAG3 antibodies (for example, said one or more immune checkpoint inhibitors may be selected from anti-CTLA-4 antibodies, anti-PD-1 antibodies and/or anti-PD-L1 antibodies, such as, e.g., ipilimumab, tremelimumab, nivolumab, pembrolizumab, cemiplimab, spartalizumab, camrelizumab, sintilimab, tislelizumab, AMP-224, AMP-514, atezolizumab, avelumab, durvalumab, KN035, or CK-301); more preferably, said one or more immune checkpoint inhibitors are selected from ipilimumab, tremelimumab, nivolumab, pembrolizumab, pidilizumab, cemiplimab, dostarlimab, spartalizumab, camrelizumab, sintilimab, tislelizumab, toripalimab, zimberelimab, AMP-224, AMP-514, JTX-4014, INCMGA00012, APE02058, atezolizumab, avelumab, durvalumab, KN035, CK-301, BMS-936559, MED14736, MPDL3280A, MDX-1105, MED16469, bintrafusp alfa, tiragolumab, vibostolimab, domvanalimab, etigilimab, BMS-986207, EOS-448, COM902, ASP8374, SEA-TGT, BGB-A1217, IBI-939, M6223, relatlimab, ieramilimab, encelimab, tebotelimab, REGN3767, FS118, IMP701, and IMP731.

The present invention thus particularly relates to a compound of formula (I) or a pharmaceutically acceptable salt or solvate thereof, or a pharmaceutical composition comprising any of the aforementioned entities optionally in combination with a pharmaceutically acceptable excipient, for use in the treatment or prevention of cancer, wherein the compound or the pharmaceutical composition is to be administered in combination with one or more anticancer drugs (including any one or more of the specific anticancer drugs described herein above).

The combinations referred to above may conveniently be presented for use in the form of a pharmaceutical formulation. The individual components of such combinations may be administered either sequentially or simultaneously/concomitantly in separate or combined pharmaceutical formulations by any convenient route. When administration is sequential, either the compound of the present invention (i.e., the compound of formula (I) or a pharmaceutically acceptable salt or solvate thereof) or the further therapeutic agent(s) may be administered first. When administration is simultaneous, the combination may be administered either in the same pharmaceutical composition or in different pharmaceutical compositions. When combined in the same formulation, it will be appreciated that the two or more compounds must be stable and compatible with each other and the other components of the formulation. When formulated separately, they may be provided in any convenient formulation and may be administered by any convenient route.

The subject or patient to be treated in accordance with the present invention may be an animal (e.g., a non-human animal). Preferably, the subject/patient is a mammal. More preferably, the subject/patient is a human (e.g., a male human or a female human) or a non-human mammal (such as, e.g., a guinea pig, a hamster, a rat, a mouse, a rabbit, a dog, a cat, a horse, a monkey, an ape, a marmoset, a baboon, a gorilla, a chimpanzee, an orangutan, a gibbon, a sheep, cattle, or a pig). Most preferably, the subject/patient to be treated in accordance with the invention is a human.

The term “treatment” of a disorder or disease, as used herein, is well known in the art. “Treatment” of a disorder or disease implies that a disorder or disease is suspected or has been diagnosed in a patient/subject. A patient/subject suspected of suffering from a disorder or disease typically shows specific clinical and/or pathological symptoms which a skilled person can easily attribute to a specific pathological condition (i.e., diagnose a disorder or disease).

The “treatment” of a disorder or disease may, for example, lead to a halt in the progression of the disorder or disease (e.g., no deterioration of symptoms) or a delay in the progression of the disorder or disease (in case the halt in progression is of a transient nature only). The “treatment” of a disorder or disease may also lead to a partial response (e.g., amelioration of symptoms) or complete response (e.g., disappearance of symptoms) of the subject/patient suffering from the disorder or disease. Accordingly, the “treatment” of a disorder or disease may also refer to an amelioration of the disorder or disease, which may, e.g., lead to a halt in the progression of the disorder or disease or a delay in the progression of the disorder or disease. Such a partial or complete response may be followed by a relapse. It is to be understood that a subject/patient may experience a broad range of responses to a treatment (such as the exemplary responses as described herein above). The treatment of a disorder or disease may, inter alia, comprise curative treatment (preferably leading to a complete response and eventually to healing of the disorder or disease) and palliative treatment (including symptomatic relief).

The term “prevention” of a disorder or disease, as used herein, is also well known in the art. For example, a patient/subject suspected of being prone to suffer from a disorder or disease may particularly benefit from a prevention of the disorder or disease. The subject/patient may have a susceptibility or predisposition for a disorder or disease, including but not limited to hereditary predisposition. Such a predisposition can be determined by standard methods or assays, using, e.g., genetic markers or phenotypic indicators. It is to be understood that a disorder or disease to be prevented in accordance with the present invention has not been diagnosed or cannot be diagnosed in the patient/subject (for example, the patient/subject does not show any clinical or pathological symptoms). Thus, the term “prevention” comprises the use of a compound of the present invention before any clinical and/or pathological symptoms are diagnosed or determined or can be diagnosed or determined by the attending physician.

It is to be understood that the present invention specifically relates to each and every combination of features and embodiments described herein, including any combination of general and/or preferred features/embodiments. In particular, the invention specifically relates to each combination of meanings (including general and/or preferred meanings) for the various groups and variables comprised in formula (I).

In this specification, a number of documents including patent applications and scientific literature are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

The reference in this specification to any prior publication (or information derived therefrom) is not and should not be taken as an acknowledgment or admission or any form of suggestion that the corresponding prior publication (or the information derived therefrom) forms part of the common general knowledge in the technical field to which the present specification relates.

The invention will now be described by reference to the following examples which are merely illustrative and are not to be construed as a limitation of the scope of the present invention.

The compounds of formula (I) described in the following examples section are defined by their chemical formulae and their corresponding chemical names. In case of conflict between any chemical formula and the corresponding chemical name indicated herein, the present invention relates to both the compound defined by the chemical formula and the compound defined by the chemical name, and particularly relates to the compound defined by the chemical formula.

### EXAMPLES

General Experimental Procedures

General Conditions

All reagents were commercial grade and used without further purification. Reactions were typically run using commercial anhydrous solvents under argon atmosphere.

Column chromatography was generally performed with a Biotage Isolera® Four apparatus using, unless stated otherwise, Biotage® KP-Sil cartridge pre-filled with 50 μm irregular silica gel. Alternatively, Interchim® PURIFLASH jumbo pack silica HP cartridges pre-filed with 50 μm silica gel (mentioned as Interchim® 50 μm), Interchim® PURIFLASH jumbo pack silica HP cartridges pre-filed with 15 μm silica gel (mentioned as Interchim® 15 μm), or Interchim® PURIFLASH jumbo pack silica SDT cartridges pre-filed with 20 μm silica gel (mentioned as Interchim® 20 μm), or Biotage Sfär® KP-Amino D cartridges pre-filed with 50 μm silica gel (mentioned as or Biotage® KPNH) could be used when necessary. Alternatively, column chromatography was performed with a Biotage Isolera Spektra One® using Silica gel 60® (40-63 μm, Merck) (mentioned as Merck 60®).

Thin layer chromatography was carried out using pre-coated silica gel F-254 plates or Biotage KP-NH TLC plates.

Releasing of free bases from the corresponding salts was carried out using Biotage ISOLUTE® SCX-2 cation exchange cartridges.

¹H-NMR spectra were recorded on a Bruker AV-300 spectrometer or on a Bruker AMX-400 spectrometer. Proton chemical shifts are listed relative to residual CD₃OD (3.31 ppm), DMSO (2.50 ppm) or D₂O (4.78 ppm). Splitting patterns are designated as s (singlet), d (doublet), dd (doublet-doublet), t (triplet), tt (triplet-triplet), td (triplet-doublet), q (quartet), quint (quintuplet), sex (sextuplet), sept (septuplet), m (multiplet), b (broad).

UPLC-MS analyses were recorded with an UPLC Waters Aquity platform with a photodiode array detector (210-400 nm) using an Acquity CSH C₁₈ 1.7 μm (2.1×30 mm) column. The mobile phase consisted in a gradient of water with 0.025% of TFA and acetonitrile with 0.025% of TFA. The flow rate was 0.8 mL per min. All analyses were performed at 55° C. The UPLC system was coupled with a Waters SQD2 platform. All mass spectra were full-scan experiments (mass range 100-800 amu) and were obtained using electrospray ionization.

HPLC-MS were recorded using an HPLC Waters platform with a 2767 sample manager, a 2525 pump, a photodiode array detector (200-400 nm). This HPLC system was coupled with a Waters Acquity QDa detector. Mass spectra were full-scan experiments (mass range 110-850 amu) and were obtained using electro spray ionization. For analytical samples, the selected column was a XSelect CSH C₁₈ 3.5 μm (2.1×30 mm) column. For preparative purifications, the selected column was, unless otherwise stated, an XSelect CSH prep C₁₈ 5 μm (19×100 mm) column. The mobile phase consisted in an appropriate gradient of water with 0.1% of formic acid and acetonitrile with 0.1% of formic acid. The flow rate was 1 mL/min in analytical mode, and in preparative mode 25 mL/min.

Alternatively, HPLC-MS were recorded using a Thermo LC/MS-Ultimate 3000-Ion Trap HCT Brucker. Mass spectra were performed on a Brucker Ion Trap and were obtained using electrospray ionization. For analytical samples, the selected column was a Nucleodur 3 μm 4.6×100 mm reverse-phase column. The mobile phase consisted in a linear gradient with a flow rate of 1.3 mL/min from 95% A and 5% B to 5% A and 95% B in 8.5 min (solvent A, H₂O with 0.1% formic acid; solvent B, acetonitrile with 0.1% formic acid). Preparative purifications were performed on a Gilson PLC 2020 apparatus using a column C8 Princeton SPHER. 60-10 μm, mentioned as Column B. The mobile phase consisted in a gradient of acetonitrile (5 to 100%) in water+0.1% formic acid with a flow rate of 30 mL/min.

After preparative HPLC, the fractions were combined, eventually partially concentrated under reduced pressure, then freeze dried from a water acetonitrile mixture.

All HPLC-MS were performed at room temperature.

Melting points were measured on a Barnstead Electrothermal 9100 or an Electrothermal 1002 and are not corrected.

Unless mentioned otherwise all compounds isolated by filtration or centrifugation were dried overnight in high vacuum at 50-70° C.

General Procedures

General Procedure (I): Ketone Addition on Halopyridine

Under inert atmosphere, to a solution of halopyridine (1.0 equiv) in THF (0.7 M) at −78° C. was added lithium diisopropylamide 1M in THF (1.2 equiv). The reaction was stirred at −78° C. for 30 min before addition of a solution of ketone (1.1 equiv) in THF (2 M). The reaction mixture was stirred at −78° C. for 1 hour. The reaction mixture was hydrolysed with NH₄Cl sat. aq. then extracted with EtOAc. The organic layer was washed with brine, dried over magnesium sulfate then concentrated to dryness. The crude was purified by flash chromatography to afford alcohol.

General Procedure (II): Dehydration

To a solution of alcohol (1.0 equiv) in acetic acid (0.4 M) was added concentrated sulfuric acid (6 equiv). The reaction was heated at 120° C. for 1 hour. The reaction mixture was cooled at 0° C., hydrolysed with water (same volume as acetic acid), basified with NaOH 15% until neutral pH then extracted with DCM. The combined organic layers were dried over magnesium sulfate and concentrated to dryness to afford alkene.

General Procedure (III): Alkene Epoxidation

To a solution of alkene (1.0 equiv) in dichloroethane (0.4 M) was added mCPBA<77% (1.5 equiv). The reaction was heated at 60° C. for 16 hours. The reaction mixture was cooled at 0° C. The white precipitate was removed by filtration then the filtrate was concentrated to dryness. The crude was purified by flash chromatography to afford epoxide.

A purification step was optionally performed in order to remove residual ketone from the previous step. To a solution of epoxide (1.0 equiv) in THF (0.6 M) at 0° C. was added sodium tetrahydroborate (0.5 equiv) then an equal volume of MeOH was added dropwise. The reaction was stirred at 25° C. for 5 min. The reaction mixture was hydrolysed with water then extracted with DCM. The organic layer was washed with brine, dried over magnesium sulfate then concentrated to dryness. The crude was purified by flash chromatography to afford purified epoxide.

General Procedure (IV): Heterocycle Formation

Under inert atmosphere, to a solution of epoxide (1.0 equiv) in DMA (0.2 M) or NMP (0.2M) was added the corresponding amine (2.0 to 3.0 equiv) and optionally DIPEA (2.0 to 3.0 equiv). The reaction mixture was subjected to microwave irradiation at 200° C. for 30 min, then hydrolysed with water and extracted with EtOAc. The organic layer was washed with brine, dried over magnesium sulfate then concentrated to dryness. The crude was purified by flash chromatography to afford heterocycle.

General Procedure (Va): Aryl Cyanation

Under inert atmosphere, to a solution of arylhalide (1.0 equiv) in DMA (0.1 M) was added Zinc(II) cyanide (1.5 equiv). The mixture was sparged with argon for 10 min before addition of Palladium tetrakis (0.1 equiv). The reaction mixture was subjected to microwave irradiation at 110° C. for 1 hour. The reaction mixture was filtered through a pad of Celite and washed with EtOAc. The organic layer was washed with NaHCO₃ sat. aq., brine, dried over magnesium sulfate and concentrated to dryness, if incomplete conversion was observed, the reaction was restarted using the same conditions, otherwise the crude was purified by flash chromatography to afford nitrile.

General Procedure (Vb): Aryl Cyanation

Under inert atmosphere, to a solution of arylhalide (1.0 equiv) in DMA (0.2 M) was added Zinc(II) cyanide (1.5 equiv). The mixture was sparged with argon for 10 min before addition of Pd(tBu₃P)₂ (0.1 equiv). The reaction mixture was subjected to microwave irradiation at 150° C. for 1 hour. The reaction mixture was filtered through a pad of Celite and washed with EtOAc. The organic layer was washed with NaHCO₃ sat. aq., brine, dried over magnesium sulfate and concentrated to dryness, if incomplete conversion was observed, the reaction was restarted using the same conditions, otherwise the crude was purified by flash chromatography to afford nitrile.

General Procedure (VI): Acid from Nitrile

To a solution of nitrile (1.0 equiv) in a 1:1:1 mixture of EtOH/Water/Dioxane (0.1 M) was added potassium hydroxide (10 equiv). The reaction mixture was heated at 100° C. for 4 days. The reaction mixture was acidified until pH=5-6 with HCl 1N aq. then extracted with EtOAc. The organic layer was washed with brine, dried over magnesium sulfate and concentrated to dryness to afford a crude which was dissolved in dioxane (0.2 M) and treated with an equal amount of HCl 1N aq., the reaction mixture was heated at 100° C. for 4 days then basified to pH=5-6 with NaOH 1N aq. and extracted with EtOAc. The organic layer was washed with brine, dried over magnesium sulfate and concentrated to dryness to afford acid which was used in the following step without further purification.

General Procedure (VII): Nitrile Hydration

To a solution of nitrile (1.0 equiv) in DMSO (0.2 M) was added potassium carbonate (1.0 equiv), and hydrogen peroxide aqueous 30% (1.5 equiv). The reaction was stirred at 25° C. for 16 hours. The reaction mixture was hydrolysed with water then extracted with EtOAc. The organic layer was washed with brine, dried over magnesium sulfate, and concentrated to dryness to afford corresponding amide which was used in the following step without further purification.

General Procedure (VIII): Methyl Ester from Amide

Under inert atmosphere, to a solution of amide (1.0 equiv) in MeOH (0.1 M) was added 1,1-dimethoxy-N,N-dimethylmethanamine (6 equiv). The reaction mixture was heated at 50 to 60° C. for 16 to 24 hours. The reaction mixture was concentrated to dryness. The crude was purified by flash chromatography to afford methyl ester.

General Procedure (IXa): Methyl Ester Hydrolysis

To a solution of methyl ester (1.0 equiv) in THF (0.1 M) was added lithium hydroxide 1M aq. (2.0 equiv). The reaction was stirred at 25° C. for 1 to 18 hours. The reaction mixture was acidified with HCl 1M aq. to pH=1 then extracted with DCM. The organic layer was dried over magnesium sulfate and concentrated to dryness to afford acid.

General Procedure (IXb): Methyl Ester Hydrolysis

To a solution of methyl ester (1.0 equiv) in THF (0.1 M) was added lithium hydroxide 1M aq. (2.0 equiv). The reaction was stirred at 25° C. for 1 to 18 hours, then concentrated under reduced pressure to afford Lithium carboxylate.

General Procedure (Xa): Peptidic Coupling

To a mixture of acid in DCM (0.1 M) was added ((1H-benzo[d][1,2,3]triazol-1-yl)oxy)tris(dimethylamino)phosphonium hexafluorophosphate(V) (1.3 equiv), N,N-diisopropylethylamine (3.0 to 4.0 equiv) and the amine (1.1 to 2.0 equiv). The reaction mixture was stirred at 25° C. for 3 to 18 hours. The reaction mixture was hydrolysed with NH₄Cl sat. aq. then extracted with DCM. The organic layer was washed with brine, dried over magnesium sulfate then concentrated to dryness to afford crude amide.

General Procedure (Xb): Peptidic Coupling

To a mixture of acid in THF (0.1 M) was added ((1H-benzo[d][1,2,3]triazol-1-yl)oxy)tris(dimethylamino)phosphonium hexafluorophosphate(V) (1.3 equiv), N,N-diisopropylethylamine (3.0 to 4.0 equiv) and the amine (1.0 to 2.0 equiv). The reaction mixture was stirred at 25° C. for 18 hours. The reaction was stirred at 25° C. for 18 hours. The reaction mixture was concentrated, diluted with EtOAc, washed with an aqueous saturated solution of NaHCO₃, and then extracted twice with EtOAc. The organic layer was dried over magnesium sulfate then concentrated to dryness to afford crude amide.

General Procedure (Xc): Peptidic Coupling

To a mixture of acid or carboxylate in DMF (0.1 M) was added ((1H-benzo[d][1,2,3]triazol-1-yl)oxy)tris(dimethylamino)phosphonium hexafluorophosphate(V) (1.3 equiv), N,N-diisopropylethylamine (3.0 to 4.0 equiv) and the amine (1.1 to 2.0 equiv). The reaction mixture was stirred at 25° C. for 3-18 hours. The reaction mixture was hydrolysed with NH₄Cl sat. aq. then extracted with EtOAc. The organic layer was washed with brine, dried over magnesium sulfate then concentrated to dryness to afford crude amide.

General Procedure (XIa): Methyl Ester Deprotection in Presence of tBuOK

To a solution of methyl ester (1.0 equiv) in tBuOH (0.1 M) was added Potassium tert-butoxide (4 equiv). The reaction mixture was heated at 80° C. for 16 hours. The reaction mixture was hydrolysed with NH₄Cl sat. aq. then extracted with DCM. The organic layer was washed with brine, dried over magnesium sulfate then concentrated to dryness to afford crude acid.

General Procedure (XIb): Methyl Ester Deprotection in Presence of tBuONa

To a solution of methyl ester (1.0 equiv) in tBuOH (0.1 M) was added Sodium tert-butoxide (10 equiv). The reaction mixture was heated at 60° C. for 18 hours. The reaction mixture was concentrated to dryness. The residue was solubilized in water, acidified with aqueous HCl 1M until pH=3, then extracted with EtOAc. The organic layer was washed with brine, dried over magnesium sulfate then concentrated to dryness to afford crude acid.

General Procedure (XIc): Methyl Ester Deprotection in Presence of Lil

To a solution of methyl ester (1.0 equiv) in Pyridine (0.1 M) was added Lithium Iodide (3-6 equiv). The reaction mixture was heated at 120° C. for 2 to 10 days. The reaction mixture was diluted with 10 volume of water and acidified with aqueous 1M HCl to pH=1, then extracted with EtOAc. The organic layer was washed with brine, dried over magnesium sulfate then concentrated to dryness to afford crude acid.

General Procedure (XII): N-tBu Cleavage

A suspension of N-tBu protected compound (1.0 equiv) in concentrated sulfuric acid (0.1 M) was stirred at 25° C. for 18 hours. The reaction mixture was hydrolysed with water then extracted with DCM. The organic layer was washed with brine, dried over magnesium sulfate then concentrated to dryness to afford N free compound which was used in the following step without further purification.

General Procedure (XIII): Nucleophilic Substitution

To a solution of nucleophile (1.0 equiv) in the appropriate solvent (0.1 M) at 0° C. was added sodium hydride (60% in mineral oil) (1.5 equiv). The reaction mixture was stirred at 0° C. for 5 minutes before addition of electrophile (1.2 equiv). The reaction mixture was stirred at 25° C. for 1 to 70 hours, or heated at an appropriate temperature for 1 to 20 hours, then hydrolysed with NH₄Cl sat. aq. and extracted with EtOAc. The organic layer was washed with brine, dried over magnesium sulfate then concentrated to dryness.

General Procedure (XIV): Heteroaryl Bromination

To a solution of heteroaryl (1.0 equiv) in DMA (0.1 M) was added N-bromosuccinimide (1.2 equiv). The reaction mixture was stirred at 25° C. for 3 hours. The reaction mixture was diluted with water (5 times the DMA volume) and the product was recovered by filtration.

General Procedure (XV): O-Silyl Deprotection

To a solution of alkoxysilane in THF (0.1 M) was added tetrabutylammonium fluoride 1M in THF (1.5 equiv). The reaction mixture was stirred at 25° C. for 2 hours. The reaction mixture was hydrolysed with NH₄Cl sat. aq. then extracted with EtOAc. The organic layer was washed with brine, dried over magnesium sulfate then concentrated to dryness. The crude was purified by flash chromatography to afford alcohol.

General Procedure (XVI): Mitsunobu Reaction

Under inert atmosphere, to a solution of alcohol (1.0 equiv) in THF (0.1 M) was added triphenylphosphine (1.5 equiv), DIAD (1.5 equiv) and corresponding nucleophile (1.5 equiv). The reaction mixture was stirred at 25° C. for 16 hours. The reaction mixture was hydrolysed then extracted with EtOAc. The organic layer was washed with brine, dried over magnesium sulfate then concentrated to dryness. The crude was purified by flash chromatography.

General Procedure (XVIIa): Suzuki Coupling

Under inert atmosphere, to a solution of halide or pseudo halide (1.0 equiv) and corresponding boronic derivatives (1.0 to 2.0 equiv) in DME (0.1 M) was added a solution of potassium carbonate 1.2M in water (3.0 equiv). The reaction mixture was sparged with Ar for 10 min before addition of Pd(dppf)Cl2 (0.1 equiv). The reaction mixture was heated at 80° C. for 2 to 20 hours. The mixture was filtered through a pad of Celite and the pad was washed with EtOAc. The organic layer was washed with water, brine and dried over magnesium sulfate then concentrated to dryness. The crude was purified by flash chromatography to afford cross-coupling product.

General Procedure (XVIIb): Suzuki Coupling

Under inert atmosphere, to a solution of halide or pseudo halide (1.0 equiv) and corresponding boronic derivatives (1.0 to 2.0 equiv) in DME (0.1 M) was added a solution of potassium carbonate 1.2M in water (3.0 equiv). The reaction mixture was sparged with Ar for 10 min before addition of Pd(dppf)Cl2 (0.1 equiv). The reaction mixture was heated at 100° C. for 1 hour. The mixture was filtered through a pad of Celite and the pad was washed with EtOAc. The organic layer was washed with water, brine and dried over magnesium sulfate then concentrated to dryness. The crude was purified by flash chromatography to afford cross-coupling product.

General Procedure (XVIIIa): Boc Deprotection

To a solution of Boc derivative in DCM (0.2 M) was added an equal volume of trifluoroacetic acid. The reaction mixture was stirred at 25° C. for 1 hour. The reaction mixture was concentrated to dryness. The residue was dissolved in HCl 1 M in MeOH (0.4 M). The solution was concentrated to dryness to obtain amine as a hydrochloride.

General Procedure (XVIIIb): Boc Deprotection

To a solution of Boc derivative in Et₂O (0.2M) was added an equal volume of HCl in Et₂O (2 M). The reaction mixture was stirred at 25° C. for 16 hours. The reaction mixture was stirred at 25° C. for 16 hours. The reaction mixture was concentrated to dryness and the crude was triturated in Et₂O (3×) to obtain amine as a HCl salt.

General Procedure (XVIIIc): Boc Deprotection

To a solution of Boc derivative in Dioxane (0.1M) was added HCl in Dioxane (4 M, 10 equiv). The reaction mixture was stirred at 25° C. for 16 hours. The reaction mixture was concentrated to dryness and the crude was triturated in Et₂O (3×) to obtain amine as a HCl salt.

General Procedure (XIXa): Heterocycle N-Oxidation

To a solution of heterocycle (1.0 equiv) in DME (0.4 M) was added 3-chlorobenzoperoxoic acid<77% (1.5 to 2.0 equiv) portionwise. The reaction mixture was stirred at 25° C. for 3 hours and concentrated to dryness. The residue was triturated in HCl aq. 2N (1.5 times the volume of DME), filtered, the filtrate was concentrated to dryness then co-evaporated twice with toluene to afford N-Oxide as a hydrochloride salt.

General Procedure (XIXb): Heterocycle N-Oxidation

To a solution of heterocycle (1.0 equiv) in DCM (0.02 M) was added 3-chlorobenzoperoxoic acid<77% (5.0 equiv). The reaction mixture was stirred at 50° C. for 90 min. The reaction mixture was washed with K₂CO₃ (aq. Sat.), the organic layer was washed with brine, dried over magnesium sulfate then concentrated to dryness. The crude residue was purified by flash chromatography to afford N-oxide.

General Procedure (XX): Nitrile Synthesis from N-Oxide

To a suspension of N-Oxide (1.0 equiv) in ACN (0.2 M) was added N,N-diisopropylethylamine (2.5 equiv) and (Trimethylsilyl)nitrile (6 equiv). The reaction mixture was subjected to microwave irradiation at 150° C. for 30 min. The reaction mixture was hydrolysed then extracted with EtOAc. The organic layer was washed with brine, dried over magnesium sulfate then concentrated to dryness to afford N-Oxide.

General Procedure (XXI): Buchwald Coupling

Under inert atmosphere, to a solution of halide or pseudohalide (1.0 equiv) and amine (1.0-1.1 equiv) in dioxane (0.1-0.2 M) was added Cesium Carbonate (3.0 equiv). The mixture was sparged with Argon for 10 min before addition of RuPhos PdG4 (0.1 equiv). The reaction mixture was heated at 100° C. for 16 hours. The reaction mixture was filtered through a pad of Celite, the pad was washed with EtOAc then the filtrate was concentrated to dryness. The crude residue was purified by flash chromatography to afford coupling product.

General Procedure (XXII): Nucleophilic Aromatic Substitution

To a solution of halide or pseudohalide (1.0 equiv) and nucleophile (1.5 equiv) in DMA (0.2 M) was added DIPEA (3.0 equiv). The reaction mixture was subjected to microwave irradiation at 150° C. for 30 minutes. The reaction mixture was diluted with water and extracted with EtOAc. The organic layer was washed with brine, dried over magnesium sulfate then concentrated to dryness. The crude was purified by flash chromatography.

General Procedure (XXIII): Alcohol Sulfonylation

Under inert atmosphere, to a solution of alcohol (1 equiv) in DCM (0.2 M) at 0° C. was added triethylamine (2 equiv) and sulfonylation reagent (1.2 to 2.0 equiv). The reaction mixture was stirred at 25° C. for 2 hours then hydrolysed and extracted with DCM. The organic layer was washed with brine, dried over magnesium sulfate and concentrated to dryness to obtain alkylating agent.

General Procedure (XXIVa): Heteroarylketone Synthesis

Under inert atmosphere, to a solution of heteroarylhalide in THF (0.2 M) at −78° C. was added a solution of ButylLithium 1.6 M in hexane (1.0 equiv). The reaction mixture was stirred at −78° C. for 30 minutes then nitrile was added and the reaction mixture was stirred at −78° C. for another 2 hours, then let warm up to 0° C. and quenched with methanolic HCl 1.25 M (1.3 equiv) and concentrated. The residue was taken up in a Methanol/HCl aq 1M solution 1:1 (0.1 M) and stirred for 3 hours at 25° C. The solution was partially concentrated under reduced pressure and extracted with DCM. The organic layer was washed with brine, dried over magnesium sulfate then concentrated to dryness. The crude was purified by flash chromatography to afford hetroarylketone.

General Procedure (XXIVb): Heteroarylketone Synthesis

Under inert atmosphere, to a solution of heteroarylhalide in THF (0.1 M) at −78° C. was added a solution of ButylLithium 2.5 M in hexane (3.0 equiv). The reaction mixture was stirred at −78° C. for 30 minutes then weinreb amide was added and the reaction mixture was stirred at −78° C. for another 2 hours, then let warm up to 25° C. over 18 hours and quenched with NH₄Cl (sat. aq.). The mixture was extracted with EtOAc, the organic layer was washed with brine, dried over magnesium sulfate then concentrated to dryness. The crude was purified by flash chromatography to afford hetroarylketone.

General Procedure (XXVa): Hydrazone Formation

Under inert atmosphere, to a solution of heteroarylketone in EtOH (0.5 M) was added H₂SO₄ 98% (0.75 equiv) and hydrazine (1.1 to 1.5 equiv). The reaction mixture was subjected to microwave irradiation at 120° C. for 30 min. The reaction mixture was concentrated to afford crude hydrazone.

General Procedure (XXVb): Hydrazone Formation

Under inert atmosphere, to a solution of heteroarylketone in THF (0.5 M) was added H₂SO₄ 98% (0.75 equiv) and hydrazine (1.1 to 1.5 equiv). The reaction mixture was subjected to microwave irradiation at 120° C. for 30 min. The reaction mixture was concentrated to afford crude hydrazone.

General Procedure (XXVI): Heteroaryl Formation Via Oxidative Cyclisation

To a solution of hydrazone in DMSO (0.15 M) was added TEMPO (1.1 equiv) and NaHCO₃ (1 equiv). The reaction mixture was heated at 150° C. for 16 hours then hydrolysed with sodium thiosulfate sat. aq. and extracted with EtOAc. The organic layer was washed with brine, dried over magnesium sulfate then concentrated to dryness. The crude was purified by flash chromatography to afford heteroaryl.

A purification step was optionally performed to remove residual ketone from the previous step. To a solution of heteroaryl (1.0 equiv) in DCM or THF (0.2 M) at 0° C. was added sodium tetrahydroborate (1.5 equiv) then an equal volume of MeOH was added dropwise. The reaction was stirred at 25° C. for 1 hour. The reaction mixture was hydrolysed with water then extracted with DCM. The organic layer was washed with brine, dried over magnesium sulfate then concentrated to dryness. The crude was purified by flash chromatography to afford purified heteroaryl.

General Procedure (XXVII): Heteroaryl Formation Via Aromatic Nuclophilic Substitution

A solution of hydrazone and K₂CO₃ (5 equiv) in NMP (0.1 M) was subjected to microwave irradiation at 150° C. for 45 min. The mixture was diluted with NH₄Cl (aq. Sat.) then extracted with EtOAc, the organic layer was washed with brine, dried over magnesium sulfate then concentrated to dryness. The crude was purified by flash chromatography to afford heteroaryl.

General Procedure (XXVIII): Chan-Lam Coupling

To a solution of N—H heterocycle in DCM (0.05 M) was added boronic acid (2.0 equiv), Cu(OAc)₂ (2.0 equiv), and pyridine (2.0 equiv). The reaction mixture was stirred at room temperature for 48 hours then concentrated. The crude residue was purified by flash chromatography to afford coupled product

General Procedure (XXIX): Aryl Carbonylation

In a bridged two-vial under inert atmosphere, in chamber 1, to a solution of halide or pseudo halide (1 equiv) in 1:1 1,4-Dioxane/MeOH mixture (0.1 M) was added triethylamine (2 equiv). In chamber 2, molybdenum hexacarbonyl (0.5 equiv) was suspended in 1,4-Dioxane (0.05 M). Both chambers were sparged with argon for 10 min. XantPhos Pd G4 (0.1 equiv) was added to chamber 1 and DBU (1.5 equiv) was added to chamber 2. The reaction was heated at 85° C. for 20 h. The mixture of chamber 1 was filtered through a pad of Celite which was washed with EtOAc. The organic layer was washed with NH4Cl sat., brine, dried over magnesium sulfate then concentrated to dryness. The crude residue was purified by flash chromatography to afford carbonylated product

General Procedure (XXX): Aryl Amino-Carbonylation

In a bridged two-vial under inert atmosphere, in chamber 1, to a solution of halide or pseudo halide (1 equiv) in 1,4-Dioxane (0.1 M) was added amine hydrochloride (2 equiv, unless otherwise stated) and triethylamine (4 equiv). In chamber 2, molybdenum hexacarbonyl (1.5 equiv) was suspended in 1,4-Dioxane (0.15 M). Both chambers were sparged with argon for 10 min. XantPhos Pd G4 (0.1 equiv) was added to chamber 1 and DBU (4.5 equiv) was added to chamber 2. The reaction was heated at 110° C. for 20 h. The mixture of chamber 1 was filtered through a pad of Celite which was washed with EtOAc. The organic layer was washed with NH4Cl sat., brine, dried over magnesium sulfate then concentrated to dryness. The crude residue was purified by flash chromatography to afford amino-carbonylated product

### Synthesis of Exemplary Compounds of the Invention

### Compound 1: 1-(4-chloro-3-fluorophenyl)-1-(2,6-dichloropyridin-3-yl)ethan-1-ol

Compound 1 was prepared according to general procedure (I) starting from 2,6-dichloropyridine (3.00 g) and 1-(4-chloro-3-fluorophenyl)ethan-1-one (3.85 g). The crude was purified by flash chromatography (CyHex 100% to CyHex/EtOAc 80:20) to obtain Compound 1 (7.13 g, n.d.) as a yellow oil. Compound 1 is contaminated with 1-(4-chloro-3-fluorophenyl)-1-(2,6-dichloropyridin-4-yl)ethan-1-ol (around 20%).

M/Z (M[³⁵Cl]₃+H)⁺: 320.0

### Compound 2: 2,6-dichloro-3-(1-(4-chloro-3-fluorophenyl)vinyl)pyridine

Compound 2 was obtained (5.98 g, n.d.) as a brown oil according to general procedure (II) starting from Compound 1 (6.51 g). Compound 2 is contaminated with 2,6-dichloro-4-(1-(4-chloro-3-fluorophenyl)vinyl)pyridine (around 20%).

M/Z (M[³⁵Cl₃]+H)⁺: 302.0

### Compound 3: 2,6-dichloro-4-(2-(4-chloro-3-fluorophenyl)oxiran-2-yl)pyridine

Compound 3 was prepared according to general procedure (III) starting from Compound 2 (5.98 g). Compound 3 (5.10 g, n.d.) was obtained after the optional purification step and purification by flash chromatography (Interchim® 50 μm, CyHex 100% to CyHex/EtOAc 90:10), as a colourless oil. Compound 3 is contaminated with 2,6-dichloro-3-(2-(4-chloro-3-fluorophenyl)oxiran-2-yl)pyridine (around 20%).

M/Z (M[³⁵Cl₃]+H)⁺: 318.0

### Compound 4: 1-(tert-butyl)-6-chloro-3-(4-chloro-3-fluorophenyl)-1H-pyrrolo[2,3-b]pyridine

Compound 4 was prepared according to general procedure (IV) starting from Compound 3 (2.00 g) and 2-methylpropan-2-amine (1.57 mL, 2.5 equiv) in DMA. The crude was purified by flash chromatography (Interchim® 50 μm, CyHex 100% to CyHex/EtOAc 95:05) to obtain Compound 4 (1.63 g, 77%) as a white solid.

M/Z (M[³⁵Cl₂]+H)⁺: 337.1

### Compound 5: 6-chloro-3-(4-chloro-3-fluorophenyl)-1-(4-methoxybenzyl)-1H-pyrrolo[2,3-b]pyridine

Compound 5 was prepared according to general procedure (IV) starting from Compound 3 (500 mg) and methoxybenzylamine (431 mg, 2.0 equiv) in DMA. The crude was purified by flash chromatography (Interchim® 50 μm, CyHex 100% to CyHex/EtOAc 90:10) to obtain Compound 5 (685 mg, n.d.) as a yellow oil.

M/Z (M[³⁵Cl₂]+H)⁺: 401.0

### Compound 6: 6-chloro-3-(4-chloro-3-fluorophenyl)-1-(3-methoxycyclobutyl)-1H-pyrrolo[2,3-b]pyridine

Compound 6 was prepared according to general procedure (IV) starting from Compound 3 (350 mg) and 3-methoxycyclobutan-1-amine hydrochloride (302 mg, 2.0 equiv) in DMA in presence of N,N-diisopropylethylamine (2.2 equiv). The crude was purified by flash chromatography (Interchim® 50 μm, CyHex 100% to CyHex/EtOAc 85:15) to obtain Compound 6 (90 mg, 22%) as a yellow oil.

M/Z (M[³⁵Cl₂]+H)⁺: 365.1

### Compound 7: 6-chloro-3-(4-chloro-3-fluorophenyl)-1-isobutyl-1H-pyrrolo[2,3-b]pyridine

Compound 7 was prepared according to general procedure (IV) starting from Compound 3 (400 mg) and isobutylamine (373 μL, 3.0 equiv) in NMP. The crude was purified by flash chromatography (Interchim®50 μm, CyHex 100% to CyHex/EtOAc 90:10) to obtain Compound 7 (297 mg, 70%) as a yellow oil.

M/Z (M[³⁵Cl₂]+H)⁺: 337.1

### Compound 8: 6-chloro-3-(4-chloro-3-fluorophenyl)-1-(tetrahydro-2H-pyran-4-yl)-1H-pyrrolo[2,3-b]pyridine

Compound 8 was prepared according to general procedure (IV) starting from Compound 3 (500 mg) and tetrahydro-2H-pyran-4-amin (318 mg, 2.0 equiv). The crude was purified by flash chromatography (Interchim® 50 μm, CyHex 100% to CyHex/EtOAc 90:10) to obtain Compound 8 (329 mg, 57%) as a yellow solid.

M/Z (M[³⁵Cl₂]+H)⁺: 365.1

### Compound 9: 6-chloro-3-(4-chloro-3-fluorophenyl)-1H-pyrrolo[2,3-b]pyridine

Compound 9 was obtained (330 mg, 99%) as a beige solid according to general procedure (XII) starting from Compound 3 (400 mg).

M/Z (M[³⁵Cl]+H)⁺: 281.0

### Compound 10: 6-chloro-3-(4-chloro-3-fluorophenyl)-1-(2,2,2-trifluoroethyl)-1H-pyrrolo[2,3-b]pyridine

Compound 10 was prepared according to general procedure (XIII) in DMA starting from Compound 9 (200 mg) and 2,2,2-Trifluoroethyl trifluoromethylsulfonate (215 mg, 3.0 equiv). The reaction mixture was stirred for 1 hour. The crude was purified by flash chromatography (Interchim® 50 μm, CyHex 100% to CyHex/EtOAc 90:10) to obtain Compound 10 (200 mg, 77%) as a yellow solid.

M/Z (M[³⁵Cl₂]+H)⁺: 363.0

### Compound 11: 6-chloro-3-(4-chloro-3-fluorophenyl)-1-methyl-1H-pyrrolo[2,3-b]pyridine

Compound 11 was prepared according to general procedure (XIII) in DMA starting from Compound 9 (75 mg) and iodomethane (49 mg, 3.0 equiv), the reaction mixture was stirred for 18 hours. The crude was purified by flash chromatography (Interchim® 50 μm, CyHex 100% to CyHex/EtOAc 90:10) to obtain Compound 11 (65 mg, 83%) as a white solid.

M/Z (M[³⁵Cl₂]+H)⁺: 295.0

### Compound 12: 6-chloro-3-(4-chloro-3-fluorophenyl)-1-(cyclopropylmethyl)-1H-pyrrolo[2,3-b]pyridine

Compound 12 was prepared according to general procedure (XIII) in DMA starting from Compound 9 (105 mg) and iodomethylcyclopropane (88 mg, 3.0 equiv). The reaction mixture was stirred for 1.5 hours. The crude was purified by flash chromatography (Interchim® 50 μm, CyHex 100% to CyHex/EtOAc 95:5) to obtain Compound 12 (98 mg, 78%) as a yellow solid.

M/Z (M[³⁵Cl₂]+H)⁺: 335.1

### Compound 13: 1-(tert-butyl)-3-(4-chloro-3-fluorophenyl)-1H-pyrrolo[2,3-b]pyridine-6-carbonitrile

Compound 13 was prepared according to general procedure (Va) starting from Compound 4 (1.63 g). The crude was purified by flash chromatography (CyHex 100% to CyHex/EtOAc 80:20) to obtain a white solid which was triturated in 20 mL of pentane to obtain Compound 13 (1.52 g, 96%) as a white solid.

M/Z (M[³⁵Cl]+H)⁺: 328.2

### Compound 14: 3-(4-chloro-3-fluorophenyl)-1-(4-methoxybenzyl)-1H-pyrrolo[2,3-b]pyridine-6-carbonitrile

Compound 14 was prepared according to general procedure (Va) starting from Compound 5 (385 mg). The crude was purified by flash chromatography (Interchim®50 μm, CyHex 100% to CyHex/EtOAc 85:15) to obtain Compound 14 (197 mg, 52%) as a yellow solid.

M/Z (M[³⁵Cl]+H)⁺: 392.1

### Compound 15: 3-(4-chloro-3-fluorophenyl)-1-(3-methoxycyclobutyl)-1H-pyrrolo[2,3-b]pyridine-6-carbonitrile

Compound 15 was prepared according to general procedure (Va) starting from Compound 6 (86 mg). The crude was purified by flash chromatography (Interchim® 50 μm, CyHex 100% to CyHex/EtOAc 80:20) to obtain Compound 15 (47 mg, 56%), as a yellow solid.

M/Z (M[³⁵Cl]+H)⁺: 356.2

### Compound 16: 3-(4-chloro-3-fluorophenyl)-1-isobutyl-1H-pyrrolo[2,3-b]pyridine-6-carbonitrile

Compound 16 was prepared according to general procedure (Va) starting from Compound 7 (290 mg). The crude residue was purified by flash chromatography (CyHex, 100% to CyHex/EtOAc 80:20) to obtain Compound 16 (205 mg, 73%) as a white solid.

M/Z (M[³⁵Cl]+H)⁺: 328.1

### Compound 17: 3-(4-chloro-3-fluorophenyl)-1-(tetrahydro-2H-pyran-4-yl)-1H-pyrrolo[2,3-b]pyridine-6-carbonitrile

Compound 17 was prepared according to general procedure (Va) starting from Compound 8 (325 mg). The crude residue was purified by flash chromatography (Interchim® 50 μm, CyHex 100% to CyHex/EtOAc 70:30) to obtain Compound 17 (226 mg, 71%) as a yellow solid.

M/Z (M[³⁵Cl]+H)⁺: 356.1

### Compound 18: 3-(4-chloro-3-fluorophenyl)-1-(2,2,2-trifluoroethyl)-1H-pyrrolo[2,3-b]pyridine-6-carbonitrile

Compound 18 was prepared according to general procedure (Va) starting from Compound 10 (195 mg). The crude was purified by flash chromatography (Interchim® 50 μm, CyHex 100% to CyHex/EtOAc 80:20) to obtain Compound 18 (175 mg, 92%) as a white solid.

M/Z (M[³⁵Cl]+H)⁺: 354.0

### Compound 19: 3-(4-chloro-3-fluorophenyl)-1-methyl-1H-pyrrolo[2,3-b]pyridine-6-carbonitrile

Compound 19 was prepared according to general procedure (Va) starting from Compound 11 (63 mg). The crude residue was purified by flash chromatography (Interchim® 50 μm, CyHex 100% to CyHex/EtOAc 85:15) to obtain Compound 19 (51 mg, 84%) as a white solid.

M/Z (M[³⁵Cl]+H)⁺: 286.0

### Compound 20: 3-(4-chloro-3-fluorophenyl)-1-(cyclopropylmethyl)-1H-pyrrolo[2,3-b]pyridine-6-carbonitrile

Compound 20 was prepared according to general procedure (Va) starting from Compound 12 (96 mg). The crude was purified by flash chromatography (Interchim®50 μm, CyHex 100% to CyHex/EtOAc 90:10) to obtain Compound 20 (75 mg, 80%) as a white solid.

M/Z (M[³⁵Cl]+H)⁺: 326.0

### Compound 21: 1H-pyrrolo[2,3-b]pyridine 7-oxide hydrochloride

Compound 21 was obtained (2.62 g, 91%) as a beige solid according to general procedure (XIXa) from 7-azaindole (2.00 g) and 3-chlorobenzoperoxoic acid<77% (6.80 g, 1.8 equiv) portionwise.

M/Z (M+H)⁺: 135.1

### Compound 22: 1H-pyrrolo[2,3-b]pyridine-6-carbonitrile

Compound 22 was prepared according to general procedure (XX) from Compound 21 (1.31 g). The crude was purified by flash chromatography (CyHex 100% to CyHex/EtOAc 85:25) to obtain Compound 22 (700 mg, 64%) as a beige solid.

M/Z (M+H)⁺: 144.0

### Compound 23: 3-bromo-1H-pyrrolo[2,3-b]pyridine-6-carbonitrile

Compound 23 was prepared according to general procedure (XIV) from Compound 22 (1.40 g) to afford Compound 23 (2.03 g, 93%) as a yellow solid.

M/Z (M[⁷⁹Br]+H)⁺: 221.7

### Compound 24: 3-bromo-1-isobutyl-1H-pyrrolo[2,3-b]pyridine-6-carbonitrile

Compound 24 was prepared according to general procedure (XIII) in DMA from Compound 23 (600 mg) and isobutyl iodide (373 μL, 1.2 equiv). The reaction mixture was stirred for 70 hours. The crude was purified by flash chromatography (CyHex 100% to CyHex/EtOAc 95:5) to obtain Compound 24 (640 mg, 85%) as a colorless oil.

M/Z (M[⁷⁹Br]+H)⁺: 278.0

### Compound 25: 1-isobutyl-3-(pyrimidin-5-yl)-1H-pyrrolo[2,3-b]pyridine-6-carbonitrile

Compound 25 was prepared according to general procedure (XVIIa) starting from Compound 24 (100 mg) and pyrimidin-5-yl boronic acid (67 mg, 1.5 equiv). The crude residue was purified by flash chromatography (Interchim® 50 μm, CyHex 100% to CyHex/EtOAc 30:70) to obtain Compound 25 (79 mg, 79%) as a beige solid.

M/Z (M+H)⁺: 278.2

### Compound 26: 1-isobutyl-3-(pyridin-4-yl)-1H-pyrrolo[2,3-b]pyridine-6-carbonitrile

Compound 26 was prepared according to general procedure (XVIIa) starting from Compound 24 (100 mg) and pyridine-4-yl boronic acid (66 mg, 1.5 equiv). The crude residue was purified by flash chromatography (Interchim® 50 μm, CyHex/EtOAc 60:40 to CyHex/EtOAc 10:90) to obtain Compound 26 (51 mg, 51%) as a brown solid.

M/Z (M+H)⁺: 277.1

### Compound 27: 2-methyl-1H-pyrrolo[2,3-b]pyridine 7-oxide hydrochloride

Compound 27 was obtained (400 mg, n.d.) as a red solid according to general procedure (XIXa) from 2-methyl-1H-pyrrolo[2,3-b]pyridine (200 mg) and 3-chlorobenzoperoxoic acid<77% (580 mg, 1.7 equiv) portionwise.

M/Z (M+H)⁺: 149.1

### Compound 28: 2-methyl-1H-pyrrolo[2,3-b]pyridine-6-carbonitrile

Compound 28 (160 mg, n.d.) as a brown solid was obtained according to general procedure (XX) from Compound 27 (185 mg) without any further purification.

M/Z (M+H)⁺: 158.0

### Compound 29: 3-bromo-2-methyl-1H-pyrrolo[2,3-b]pyridine-6-carbonitrile

Compound 29 was prepared according to general procedure (XIV) from Compound 28 (160 mg). The crude was purified by flash chromatography (Interchim® 50 μm, CyHex 100% to CyHex/EtOAc 80:20) to obtain Compound 29 (85 mg, 24%, yield over three steps) as a yellow solid.

M/Z (M[⁷⁹Br]+H)⁺: 236.0

### Compound 30: 3-bromo-1-isobutyl-2-methyl-1H-pyrrolo[2,3-b]pyridine-6-carbonitrile

Compound 30 was prepared according to general procedure (XIII) in DMA from Compound 29 (82 mg) and isobutyl iodide (52 μL, 1.2 equiv). The reaction mixture was stirred for 18 hours. The crude was purified by flash chromatography (Interchim® 50 μm, CyHex 100% to CyHex/EtOAc 90:10) to obtain Compound 30 (73 mg, 71%) as a beige solid.

M/Z (M[⁷⁹Br]+H)⁺: 292.0

### Compound 31: 3-(4-chloro-3-fluorophenyl)-1-isobutyl-2-methyl-1H-pyrrolo[2,3-b]pyridine-6-carbonitrile

Compound 31 was prepared according to general procedure (XVIIa) starting from Compound 30 (70 mg) and (4-chloro-3-fluorophenyl) boronic acid (63 mg, 1.5 equiv). The crude residue was purified by flash chromatography (Interchim® 50 μm, CyHex 100% to CyHex/EtOAc 95:5) to obtain Compound 31 (68 mg, 83%) as a beige solid.

M/Z (M[³⁵Cl]+H)⁺: 342.1

### Compound 32: 2-(2,2,2-trifluoroethoxy)ethyl methanesulfonate

Compound 32 was obtained (340 mg, n.d.) as a brown oil according to general procedure (XXIII) from 2-(2,2,2-trifluoroethoxy)ethan-1-ol (207 mg) and methanesulfonyl chloride (134 μL, 1.2 equiv).

¹H-NMR (DMSO-d₆, 400 MHz) δ: 3.24 (s, 3H, CH₃); 3.87 (m, 2H, O—CH₂); 4.13 (q, J 9.2 Hz, 2H, O—CH₂—CF₃); 4.35 (m, 2H, O—CH₂).

### Compound 33: 3-bromo-1-(2-(2,2,2-trifluoroethoxy)ethyl)-1H-pyrrolo[2,3-b]pyridine-6-carbonitrile

Compound 33 was prepared according to general procedure (XIII) in DMA from Compound 23 (160 mg) and Compound 32 (320 mg, 2 equiv). The reaction mixture was heated at 70° C. for 1 hour. The crude was purified by flash chromatography (Interchim® 50 μm, CyHex 100% to CyHex/EtOAc 90:10) to obtain Compound 33 (161 mg, 64%) as a colorless oil.

M/Z (M[⁷⁹Br]+H)⁺: 347.9

### Compound 34: 3-(4-chloro-3-fluorophenyl)-1-(2-(2,2,2-trifluoroethoxy)ethyl)-1H-pyrrolo[2,3-b]pyridine-6-carbonitrile

Compound 34 was prepared according to general procedure (XVIIa) starting from Compound 33 (155 mg) and (4-chloro-3-fluorophenyl) boronic acid (93 mg, 1.2 equiv). The crude residue was purified by flash chromatography (Interchim® 50 μm, CyHex 100% to CyHex/EtOAc 80:20) to obtain Compound 34 (121 mg, 68%) as a yellow solid.

M/Z (M[³⁵Cl]+H)⁺: 398.1

### Compound 35: 3-bromo-1-(2-((tert-butyldimethylsilyl)oxy)ethyl)-1H-pyrrolo[2,3-b]pyridine-6-carbonitrile

Compound 35 was prepared according to general procedure (XIII) in DMA from Compound 23 (200 mg) and (2-bromoethoxy)(tert-butyl)dimethylsilane (232 μL, 1.2 equiv). The reaction mixture was stirred for 66 hours. The crude was purified by flash chromatography (Interchim® 50 μm, CyHex 100% to CyHex/EtOAc 95:5) to obtain Compound 35 (252 mg, 74%) as a white solid.

M/Z (M[⁷⁹Br]+H)⁺: 380.1

### Compound 36: 3-bromo-1-(2-hydroxyethyl)-1H-pyrrolo[2,3-b]pyridine-6-carbonitrile

Compound 36 was prepared according to general procedure (XV) from Compound 35 (250 mg). The crude was purified by flash chromatography (Interchim® 50 μm, CyHex 100% to CyHex/EtOAc 50:50) to obtain Compound 36 (162 mg, 93%) as a white solid.

M/Z (M[⁷⁹Br]+H)⁺: 266.0

### Compound 37: 1-(2-(1H-1,2,4-triazol-1-yl)ethyl)-3-bromo-1H-pyrrolo[2,3-b]pyridine-6-carbonitrile

Compound 37 was prepared according to general procedure (XVI) from Compound 36 (150 mg) and 1,2,4-triazole (58 mg, 1.5 equiv). The crude was purified by flash chromatography (Interchim® 50 μm, CyHex/EtOAC 70:30 to EtOAc 100%) to obtain Compound 37 (185 mg, n.d.) as a white solid.

M/Z (M[⁷⁹Br]+H)⁺: 316.9

### Compound 38: 1-(2-(1H-1,2,4-triazol-1-yl)ethyl)-3-(4-chloro-3-fluorophenyl)-1H-pyrrolo[2,3-b]pyridine-6-carbonitrile

Compound 38 was prepared according to general procedure (XVIIa) starting from Compound 37 (185 mg) and (4-chloro-3-fluorophenyl) boronic acid (122 mg, 1.2 equiv). The crude residue was purified by flash chromatography (Interchim® 50 μm, CyHex/EtOAc 60:40 to EtoAc 100%) to obtain Compound 38 (152 mg, 71%) as an orange solid.

M/Z (M[³⁵Cl]+H)⁺: 367.0

### Compound 39: 2-(2-oxopyrrolidin-1-yl)ethyl methanesulfonate

Compound 39 was obtained (330 mg, n.d.) as a yellow oil according to general procedure (XXIII) from 1-(2-hydroxyethyl)pyrrolidin-2-one (192 mg) and methanesulfonyl chloride (138 μL, 1.2 equiv).

¹H-NMR (DMSO-d₆, 400 MHz) δ: 1.95 (m, 2H, N—CH₂—CH₂—CH₂); 2.22 (t, J 8.2 Hz, 2H, N—CH₂); 3.18 (s, 3H, CH₃); 3.40 (t, J 7.0 Hz, 2H, CH₂—C(O)); 3.50 (t, J 6.0 Hz, 2H, N—CH₂—CH₂—O); 4.28 (t, J 6.0 Hz, 2H, N—CH₂—CH₂—O).

### Compound 40: 3-bromo-1-(2-(2-oxopyrrolidin-1-yl)ethyl)-1H-pyrrolo[2,3-b]pyridine-6-carbonitrile

Compound 40 was prepared according to general procedure (XIII) in DMA from Compound 23 (165 mg) and Compound 39 (308 mg, 2 equiv). The reaction mixture was heated at 70° C. for 1 hour. The crude was purified by flash chromatography (Interchim® 50 μm, CyHex/EtOAc 50:50 to EtOAc 100%) to obtain Compound 40 (154 mg, 62%) as a beige solid.

M/Z (M[⁷⁹Br]+H)⁺: 333.0

### Compound 41: 3-(4-chloro-3-fluorophenyl)-1-(2-(2-oxopyrrolidin-1-yl)ethyl)-1H-pyrrolo[2,3-b]pyridine-6-carbonitrile

Compound 41 was prepared according to general procedure (XVIIa) starting from Compound 40 (150 mg) and (4-chloro-3-fluorophenyl) boronic acid (94 mg, 1.2 equiv). The crude residue was purified by flash chromatography (Interchim® 50 μm, CyHex/EtOAc 50:50 to EtOAc 100%) to obtain Compound 41 (106 mg, 62%) as a beige solid.

M/Z (M[³⁵Cl]+H)⁺: 383.1

### Compound 42: 3-bromo-1-(2-ethoxyethyl)-1H-pyrrolo[2,3-b]pyridine-6-carbonitrile

Compound 42 was prepared according to general procedure (XIII) in DMA from Compound 36 (150 mg) and iodoethane (55 μL, 1.2 equiv). The reaction mixture was stirred for 3 hours. The crude was purified by flash chromatography (Interchim® 50 μm, CyHex 100% to CyHex/EtOAc 80:20) to obtain Compound 42 (112 mg, 68%) as a white solid.

M/Z (M[⁷⁹Br]+H)⁺: 293.9

### Compound 43: 3-(4-chloro-3-fluorophenyl)-1-(2-ethoxyethyl)-1H-pyrrolo[2,3-b]pyridine-6-carbonitrile

Compound 43 was prepared according to general procedure (XVIIa) starting from Compound 42 (110 mg) and (4-chloro-3-fluorophenyl) boronic acid (78 mg, 1.2 equiv). The crude was purified by flash chromatography (Interchim® 50 μm, CyHex 100% to CyHex/EtOAc 80:20) to obtain Compound 43 (84 mg, 65%) as a beige solid.

M/Z (M[³⁵Cl]+H)⁺: 344.1

### Compound 44: 1-(6-chloropyridin-2-yl)-2,2-dimethylpropan-1-one

Compound 44 was prepared according to general procedure (XXIVa) starting from 2-bromo-6-chloropyridine (2.58 g) and pivalonitrile (1.8 mL, 1.2 equiv). The crude was purified by flash chromatography (CyHex 100% to CyHex/EtOAc 90:10) to obtain Compound 44 (2.05 g, n.d.) as a yellow oil.

M/Z ([³⁵Cl]M+H)⁺: 198.1

### Compound 45: 1:1 E/Z mixture of 2-chloro-6-(1-(2-(4-chloro-3-fluorophenyl)hydrazineylidene)-2,2-dimethylpropyl)pyridine

Compound 45 was prepared according to general procedure (XXVa) starting from Compound 44 (1.05 g) and 4-chloro-3-fluorophenyl)hydrazine (938 mg, 1.1 equiv). The crude was purified by flash chromatography (CyHex 100% to CyHex/EtOAc 93:7) to obtain Compound 45 (1.81 g, n.d.) as an orange oil.

M/Z ([³⁵Cl₂]+H)⁺: 340.2

### Compound 46: 3-(tert-butyl)-5-chloro-1-(4-chloro-3-fluorophenyl)-1H-pyrazolo[4,3-b]pyridine

Compound 46 was prepared according to general procedure (XXVI) starting from Compound 45 (600 mg). Compound 46 (380 mg, 63%) was obtained, after the optional purification step and purification by flash chromatography (100% to CyHex/EtOAc 95:05), as a yellow oil.

M/Z (M[³⁵Cl₂]+H)⁺: 338.1

### Compound 47: 3-(tert-butyl)-1-(4-chloro-3-fluorophenyl)-1H-pyrazolo[4,3-b]pyridine-5-carbonitrile

Compound 47 was prepared according to general procedure (Va) starting from Compound 46 (380 mg). The crude was purified by flash chromatography (Interchim®50 μm, CyHex 100% to CyHex/EtOAc 90:10) to obtain Compound 47 (273 mg, 74%) as a white solid.

M/Z (M[³⁵Cl]+H)⁺: 329.1

### Compound 48: 1-(tert-butyl)-3-(4-chloro-3-fluorophenyl)-1H-pyrrolo[2,3-b]pyridine-6-carboxamide

Compound 48 was obtained (1.59 g, 100%) as a yellow solid according to general procedure (VII) starting from Compound 13 (1.51 g).

M/Z (M[³⁵Cl]+H)⁺: 346.0

### Compound 49: 3-(4-chloro-3-fluorophenyl)-1-(4-methoxybenzyl)-1H-pyrrolo[2,3-b]pyridine-6-carboxamide

Compound 49 was obtained (131 mg, 93%), as a yellow solid according to general procedure (VII) starting from Compound 14 (135 mg).

M/Z (M[³⁵Cl]+H)⁺: 410.1

### Compound 50: 3-(4-chloro-3-fluorophenyl)-1-(3-methoxycyclobutyl)-1H-pyrrolo[2,3-b]pyridine-6-carboxamide

Compound 50 was obtained (47 mg, 99%) as a yellow solid according to general procedure (VII) starting from Compound 15 (45 mg).

M/Z (M[³⁵Cl]+H)⁺: 374.2

### Compound 51: 3-(4-chloro-3-fluorophenyl)-1-isobutyl-1H-pyrrolo[2,3-b]pyridine-6-carboxamide

Compound 51 was obtained (188 mg, 99%) as a yellow solid according to general procedure (VII) starting from Compound 16 (180 mg).

M/Z (M[³⁵Cl]+H)⁺: 346.0

### Compound 52: 3-(4-chloro-3-fluorophenyl)-1-(tetrahydro-2H-pyran-4-yl)-1H-pyrrolo[2,3-b]pyridine-6-carboxamide

Compound 52 was obtained (235 mg, 99%) as a yellow solid according to general procedure (VII) starting from Compound 17 (225 mg).

M/Z (M[³⁵Cl]+H)⁺: 374.1

### Compound 53: 3-(4-chloro-3-fluorophenyl)-1-(2,2,2-trifluoroethyl)-1H-pyrrolo[2,3-b]pyridine-6-carboxamide

Compound 53 was obtained (157 mg, 86%) as a yellow solid according to general procedure (VII) starting from Compound 18 (173 mg).

M/Z (M[³⁵Cl]+H)⁺: 372.0

### Compound 54: 3-(4-chloro-3-fluorophenyl)-1-methyl-1H-pyrrolo[2,3-b]pyridine-6-carboxamide

Compound 54 was obtained (51 mg, 98%) as a yellow solid according to general procedure (VII) starting from Compound 19 (49 mg).

M/Z (M[³⁵Cl]+H)⁺: 304.1

### Compound 55: 3-(4-chloro-3-fluorophenyl)-1-(cyclopropylmethyl)-1H-pyrrolo[2,3-b]pyridine-6-carboxamide

Compound 55 was obtained (76 mg, 99%) as a beige solid according to general procedure (VII) starting from Compound 20 (73 mg).

M/Z (M[³⁵Cl]+H)⁺: 344.1

### Compound 56: 1-isobutyl-3-(pyrimidin-5-yl)-1H-pyrrolo[2,3-b]pyridine-6-carboxamide

Compound 56 was obtained (80 mg, 99%) as a yellow solid according to general procedure (VII) starting from Compound 25 (76 mg).

M/Z (M+H)⁺: 296.1

### Compound 57: 1-isobutyl-3-(pyridin-4-yl)-1H-pyrrolo[2,3-b]pyridine-6-carboxamide

Compound 57 was obtained (43 mg, 82%) as a beige solid according to general procedure (VII) starting from Compound 26 (49 mg) to obtain Compound 57 (43 mg, 82%) as a beige solid.

M/Z (M+H)⁺: 295.1

### Compound 58: 3-(4-chloro-3-fluorophenyl)-1-isobutyl-2-methyl-1H-pyrrolo[2,3-b]pyridine-6-carboxamide

Compound 58 was obtained (61 mg, 89%) as a white solid according to general procedure (VII) starting from Compound 31 (65 mg).

M/Z (M[³⁵Cl]+H)⁺: 360.0

### Compound 59: 3-(4-chloro-3-fluorophenyl)-1-(2-(2,2,2-trifluoroethoxy)ethyl)-1H-pyrrolo[2,3-b]pyridine-6-carboxamide

Compound 59 was obtained (112 mg, 91%) as a beige solid according to general procedure (VII) starting from Compound 34 (118 mg).

M/Z (M[³⁵Cl]+H)⁺: 416.0

### Compound 60: 1-(2-(1H-1,2,4-triazol-1-yl)ethyl)-3-(4-chloro-3-fluorophenyl)-1H-pyrrolo[2,3-b]pyridine-6-carboxamide

Compound 60 was obtained (156 mg, 99%) as a brown solid according to general procedure (VII) starting from Compound 38 (150 mg).

M/Z (M[³⁵Cl]+H)⁺: 385.1

### Compound 61: 3-(4-chloro-3-fluorophenyl)-1-(2-(2-oxopyrrolidin-1-yl)ethyl)-1H-pyrrolo[2,3-b]pyridine-6-carboxamide

Compound 61 was obtained (92 mg, 85%) as a yellow solid according to general procedure (VII) starting from Compound 41 (103 mg).

M/Z (M[³⁵Cl]+H)⁺: 401.1

### Compound 62: 3-(4-chloro-3-fluorophenyl)-1-(2-ethoxyethyl)-1H-pyrrolo[2,3-b]pyridine-6-carboxamide

Compound 62 was obtained (85 mg, 98%) as a yellow solid according to general procedure (VII) starting from Compound 43 (82 mg).

M/Z (M[³⁵Cl]+H)⁺: 362.0

### Compound 63: methyl 1-(tert-butyl)-3-(4-chloro-3-fluorophenyl)-1H-pyrrolo[2,3-b]pyridine-6-carboxylate

Compound 63 was prepared according to general procedure (VIII) starting from Compound 48 (1.59 g). The crude was purified by flash chromatography (CyHex 100% to CyHex/EtOAc 90:10) to obtain Compound 63 (1.37 g, 83%) as a white solid.

M/Z (M[³⁵Cl]+H)⁺: 361.1

### Compound 64: methyl 3-(4-chloro-3-fluorophenyl)-1-(4-methoxybenzyl)-1H-pyrrolo[2,3-b]pyridine-6-carboxylate

Compound 64 was prepared according to general procedure (VIII) starting from Compound 49 (115 mg). The crude was purified by flash chromatography (Interchim® 50 μm, CyHex 100% to CyHex/EtOAc 80:20) to obtain Compound 64 (106 mg, 89%) as a beige solid.

M/Z (M[³⁵Cl]+H)⁺: 425.2

### Compound 65: methyl 3-(4-chloro-3-fluorophenyl)-1-(3-methoxycyclobutyl)-1H-pyrrolo[2,3-b]pyridine-6-carboxylate

Compound 65 was prepared according to general procedure (VIII) starting from Compound 50 (45 mg). The crude was purified by flash chromatography (Interchim® 50 μm, CyHex 100% to CyHex/EtOAc 60:40) to obtain Compound 65 (44 mg, 94%) as a yellow oil.

M/Z (M[³⁵Cl]+H)⁺: 389.2

### Compound 66: methyl 3-(4-chloro-3-fluorophenyl)-1-isobutyl-1H-pyrrolo[2,3-b]pyridine-6-carboxylate

Compound 66 was prepared according to general procedure (VIII) starting from Compound 51 (180 mg). The crude was purified by flash chromatography (Interchim® 50 μm, CyHex 100% to CyHex/EtOAc 80:20) to obtain Compound 66 (172 mg, 92%) as a yellow solid.

M/Z (M[³⁵Cl]+H)⁺: 361.0.

### Compound 67: methyl 3-(4-chloro-3-fluorophenyl)-1-(tetrahydro-2H-pyran-4-yl)-1H-pyrrolo[2,3-b]pyridine-6-carboxylate

Compound 67 was prepared according to general procedure (VIII) starting from Compound 52 (225 mg). The crude was purified by flash chromatography (Interchim® 50 μm, CyHex 100% to CyHex/EtOAc 60:40) to obtain Compound 67 (207 mg, 88%) as a beige solid.

M/Z (M[³⁵Cl]+H)⁺: 389.2

### Compound 68: methyl 3-(4-chloro-3-fluorophenyl)-1-(2,2,2-trifluoroethyl)-1H-pyrrolo[2,3-b]pyridine-6-carboxylate

Compound 68 was prepared according to general procedure (VIII) starting from Compound 53 (155 mg). The crude was purified by flash chromatography (Interchim® 50 μm, CyHex 100% to CyHex/EtOAc 80:20 to obtain Compound 68 (130 mg, 81%) as a white solid.

M/Z (M[³⁵Cl]+H)⁺: 387.0

### Compound 69: methyl 3-(4-chloro-3-fluorophenyl)-1-methyl-1H-pyrrolo[2,3-b]pyridine-6-carboxylate

Compound 69 was prepared according to general procedure (VIII) starting from Compound 54 (50 mg, 1.0 equiv). The crude was purified by flash chromatography (Interchim® 50 μm, CyHex 100% to CyHex/EtOAc 50:50) to obtain Compound 69 (44 mg, 84%) as a white solid.

M/Z (M[³⁵Cl]+H)⁺: 319.0

### Compound 70: methyl 3-(4-chloro-3-fluorophenyl)-1-(cyclopropylmethyl)-1H-pyrrolo[2,3-b]pyridine-6-carboxylate

Compound 70 was prepared according to general procedure (VIII) starting from Compound 55 (76 mg). The crude was purified by flash chromatography (Interchim® 50 μm, CyHex 100% to CyHex/EtOAc 80:20) to obtain Compound 70 (72 mg, 91%) as a beige solid.

M/Z (M[³⁵Cl]+H)⁺: 359.1

### Compound 71: methyl 1-isobutyl-3-(pyrimidin-5-yl)-1H-pyrrolo[2,3-b]pyridine-6-carboxylate

Compound 71 was prepared according to general procedure (VIII) starting from Compound 56 (80 mg). The crude was purified by flash chromatography (Interchim® 50 μm, CyHex 100% to CyHex/EtOAc 30:70) to obtain Compound 71 (63 mg, 75%) as a beige solid.

M/Z (M+H)⁺: 311.1

### Compound 72: methyl 1-isobutyl-3-(pyridin-4-yl)-1H-pyrrolo[2,3-b]pyridine-6-carboxylate

Compound 72 was prepared according to general procedure (VIII) starting from Compound 57 (43 mg). The crude was purified by flash chromatography (Interchim® 50 μm, CyHex 100% to CyHex/EtOAc 30:70) to obtain Compound 72 (29 mg, 64%) as a yellow solid.

M/Z (M+H)⁺: 310.0

### Compound 73: methyl 3-(4-chloro-3-fluorophenyl)-1-isobutyl-2-methyl-1H-pyrrolo[2,3-b]pyridine-6-carboxylate

Compound 73 was prepared according to general procedure (VIII) starting from Compound 57 (58 mg). The crude was purified by flash chromatography (Interchim® 50 μm, CyHex 100% to CyHex/EtOAc 80:20) to obtain Compound 73 (43 mg, 71%) as a beige solid.

M/Z (M[³⁵Cl]+H)⁺: 375.0

### Compound 74: methyl 3-(4-chloro-3-fluorophenyl)-1-(2-(2,2,2-trifluoroethoxy)ethyl)-1H-pyrrolo[2,3-b]pyridine-6-carboxylate

Compound 74 was prepared according to general procedure (VIII) starting from Compound 59 (110 mg). The crude was purified by flash chromatography (Interchim® 50 μm, CyHex 100% to CyHex/EtOAc 50:50) to obtain Compound 74 (107 mg, 94%) as a yellow solid.

M/Z (M[³⁵Cl]+H)⁺: 431.0

### Compound 75: 1-(2-(1H-1,2,4-triazol-1-yl)ethyl)-3-(4-chloro-3-fluorophenyl)-1H-pyrrolo[2,3-b]pyridine-6-carboxylate

Compound 75 was prepared according to general procedure (VIII) starting from Compound 60 (155 mg). The crude was purified by flash chromatography (Interchim® 50 μm, DCM 100% to DCM/MeOH 95:5) to obtain Compound 60 (70 mg, 43%) as a yellow solid.

M/Z (M[³⁵Cl]+H)⁺: 400.1

### Compound 76: methyl 3-(4-chloro-3-fluorophenyl)-1-(2-(2-oxopyrrolidin-1-yl)ethyl)-1H-pyrrolo[2,3-b]pyridine-6-carboxylate

Compound 76 was prepared according to general procedure (VIII) starting from Compound 61 (90 mg). The crude was purified by flash chromatography (Interchim® 50 μm, DCM 100% to DCM/MeOH 95:5) to obtain Compound 76 (76 mg, 81%) as a yellow solid.

M/Z (M[³⁵Cl]+H)⁺: 416.1

### Compound 77: methyl 3-(4-chloro-3-fluorophenyl)-1-(2-ethoxyethyl)-1H-pyrrolo[2,3-b]pyridine-6-carboxylate

Compound 77 was prepared according to general procedure (VIII) starting from Compound 62 (86 mg). The crude was purified by flash chromatography (Interchim® 50 μm, CyHex 100% to CyHex/EtOAc 50:50) to obtain Compound 77 (69 mg, 77%) as a yellow solid.

M/Z (M[³⁵Cl]+H)⁺: 377.0

### Compound 78: 1-(tert-butyl)-3-(4-chloro-3-fluorophenyl)-1H-pyrrolo[2,3-b]pyridine-6-carboxylic acid

Compound 78 was obtained (1.31 g, 99%) as a yellow solid according to general procedure (IXa) starting from Compound 63 (1.37 g).

M/Z (M[³⁵Cl]+H)⁺: 347.1

### Compound 79: 3-(4-chloro-3-fluorophenyl)-1-(4-methoxybenzyl)-1H-pyrrolo[2,3-b]pyridine-6-carboxylic acid

Compound 79 was obtained (90 mg, 99%) as a beige solid according to general procedure (IXa) starting from Compound 64 (94 mg).

M/Z (M[³⁵Cl]+H)⁺: 411.2

### Compound 80: 3-(4-chloro-3-fluorophenyl)-1-(3-methoxycyclobutyl)-1H-pyrrolo[2,3-b]pyridine-6-carboxylic acid

Compound 80 was obtained (33 mg, 82%) as a beige solid according to general procedure (IXa) starting from Compound 65 (42 mg).

M/Z (M[³⁵Cl]+H)⁺: 375.1

### Compound 81: 3-(4-chloro-3-fluorophenyl)-1-isobutyl-1H-pyrrolo[2,3-b]pyridine-6-carboxylic acid

Compound 81 was obtained (145 mg, 99%) as a yellow solid according to general procedure (IXa) starting from Compound 66 (152 mg).

M/Z (M[³⁵Cl]+H)⁺: 347.1

### Compound 82: 3-(4-chloro-3-fluorophenyl)-1-(tetrahydro-2H-pyran-4-yl)-1H-pyrrolo[2,3-b]pyridine-6-carboxylic acid

Compound 82 was obtained (176 mg, 99%) as a white solid according to general procedure (IXa) starting from Compound 67 (185 mg).

M/Z (M[³⁵Cl]+H)⁺: 375.1

### Compound 83: 3-(4-chloro-3-fluorophenyl)-1-(2,2,2-trifluoroethyl)-1H-pyrrolo[2,3-b]pyridine-6-carboxylic acid

Compound 83 was obtained (124 mg, 99%) as a white solid according to general procedure (IXa) starting from Compound 68 (130 mg).

M/Z (M[³⁵Cl]+H)⁺: 373.0

### Compound 84: 3-(4-chloro-3-fluorophenyl)-1-methyl-1H-pyrrolo[2,3-b]pyridine-6-carboxylic acid

Compound 84 was obtained (63 mg, 97%) as a yellow solid according to general procedure (IXa) starting from Compound 69 (68 mg).

M/Z (M[³⁵Cl]+H)⁺: 305.0

### Compound 85: 3-(4-chloro-3-fluorophenyl)-1-(cyclopropylmethyl)-1H-pyrrolo[2,3-b]pyridine-6-carboxylic acid

Compound 85 was obtained (66 mg, 98%) as a yellow solid according to general procedure (IXa) starting from Compound 70 (70 mg).

M/Z (M[³⁵Cl]+H)⁺: 345.1

### Compound 86: lithium 1-isobutyl-3-(pyrimidin-5-yl)-1H-pyrrolo[2,3-b]pyridine-6-carboxylate

Compound 86 was obtained (100 mg, n.d.) as a white solid according to general procedure (IXb) starting from Compound 71 (60 mg).

M/Z (M+H)⁺: 297.1

### Compound 87: lithium 1-isobutyl-3-(pyridin-4-yl)-1H-pyrrolo[2,3-b]pyridine-6-carboxylate

Compound 87 was obtained (60 mg, n.d.) as a white solid according to general procedure (IXb) starting from Compound 72 (26 mg).

M/Z (M+H)⁺: 296.1

### Compound 88: lithium 3-(4-chloro-3-fluorophenyl)-1-isobutyl-2-methyl-1H-pyrrolo[2,3-b]pyridine-6-carboxylate

Compound 88 was obtained (80 mg, n.d.) as a white solid according to general procedure (IXb) starting from Compound 73 (40 mg).

M/Z (M[³⁵Cl]+H)⁺: 361.0

### Compound 89: 3-(4-chloro-3-fluorophenyl)-1-(2-(2,2,2-trifluoroethoxy)ethyl)-1H-pyrrolo[2,3-b]pyridine-6-carboxylic acid

Compound 89 was obtained (87 mg, 86%) as a yellow solid according to general procedure (IXa) starting from Compound 74 (105 mg).

M/Z (M[³⁵Cl]+H)⁺: 417.0

### Compound 90: 1-(2-(1H-1,2,4-triazol-1-yl)ethyl)-3-(4-chloro-3-fluorophenyl)-1H-pyrrolo[2,3-b]pyridine-6-carboxylic acid

Compound 90 was obtained (40 mg, 64%) as a yellow solid according to general procedure (IXa) starting from Compound 75 (65 mg).

M/Z (M[³⁵Cl]+H)⁺: 386.1

### Compound 91: lithium 3-(4-chloro-3-fluorophenyl)-1-(2-(2-oxopyrrolidin-1-yl)ethyl)-1H-pyrrolo[2,3-b]pyridine-6-carboxylate

Compound 91 was obtained (85 mg, n.d.) as a beige solid according to general procedure (IXb) starting from Compound 76 (74 mg).

M/Z (M[³⁵Cl]+H)⁺: 402.0

### Compound 92: 3-(4-chloro-3-fluorophenyl)-1-(2-ethoxyethyl)-1H-pyrrolo[2,3-b]pyridine-6-carboxylic acid

Compound 92 was obtained (62 mg, 99%) as a yellow solid according to general procedure (IXa) starting from Compound 77 (65 mg).

M/Z (M[³⁵Cl]+H)⁺: 363.0

### Compound 93: 3-(tert-butyl)-1-(4-chloro-3-fluorophenyl)-1H-pyrazolo[4,3-b]pyridine-5-carboxylic acid

Compound 93 was obtained (260 mg, 98%) as a white solid according to general procedure (VI) starting from Compound 47 (250 mg).

M/Z (M[³⁵Cl]+H)⁺: 348.2

### Compound 94: tert-butyl 4-(5-(methoxycarbonyl)-4,6-dimethylpyridin-2-yl)-2,2-dimethylpiperazine-1-carboxylate

Compound 94 was prepared according to general procedure (XXI) starting from methyl 6-chloro-2,4-dimethylnicotinate (570 mg) and tert-butyl 2,2-dimethylpiperazine-1-carboxylate (612 mg). The crude residue was purified by flash chromatography (CyHex 100% to CyHex/EtOAc 50:50) to obtain Compound 94 (781 mg, 73%) as a beige solid.

M/Z (M+H)⁺: 377.3

### Compound 95: methyl 6-(3,3-dimethylpiperazin-1-yl)-2,4-dimethylnicotinate hydrochloride

Compound 95 was obtained (690 mg, n.d.) as a yellow solid according to general procedure (XVIIIa) starting from Compound 94 (775 mg).

M/Z (M+H)⁺: 278.2

### Example 1: 4-(1-(tert-butyl)-3-(4-chloro-3-fluorophenyl)-1H-pyrrolo[2,3-b]pyridine-6-carbonyl)-3,3-dimethylpiperazin-2-one

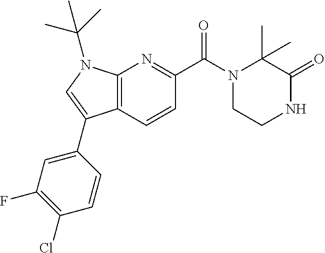 [SMILES: CC1(C)C(=O)NCCN1C(=O)c1ccc2c(-c3ccc(Cl)c(F)c3)cn(C(C)(C)C)c2n1]

Example 1 was prepared according to general procedure (Xa) starting from Compound 78 (500 mg) and 3,3-dimethylpiperazin-2-one (203 mg, 1.1 equiv), and using N,N-diisopropylethylamine (3.0 equiv). The crude was purified by preparative HPLC (40% to 80% ACN/H₂O), to obtain Example 1 (568 mg, 78%) as a white solid.

¹H-NMR (DMSO-d₆, 400 MHz) δ: 1.74 (s, 6H, 2 CH₃); 1.80 (s, 9H, 3 CH₃); 3.31 (m, 2H, CH₂); 3.57-3.59 (m, 2H, CH₂); 7.44 (d, J 8.3 Hz, 1H, Ar); 7.60-7.67 (m, 2H, Ar); 7.80-7.83 (dd, J 10.9, 1.8 Hz, 1H, Ar); 8.13 (m, 1H, Ar); 8.21 (s, 1H, Ar); 8.43 (d, J 8.3 Hz, 1H, Ar). 2H not observed.

M/Z (M[³⁵Cl]+H)⁺: 457.2

Mp: 68-80° C.

### Example 2: 4-(1-(tert-butyl)-3-(4-chloro-3-fluorophenyl)-1H-pyrrolo[2,3-b]pyridine-6-carbonyl)-1,3,3-trimethylpiperazin-2-one

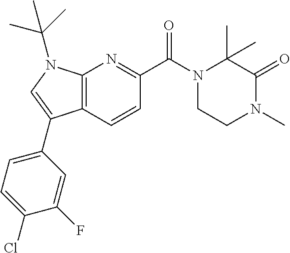 [SMILES: CN1CCN(C(=O)c2ccc3c(-c4ccc(Cl)c(F)c4)cn(C(C)(C)C)c3n2)C(C)(C)C1=O]

Example 2 was prepared according to general procedure (XIII) in DMA starting from Example 1 (75 mg) and iodomethane (28 mg, 1.2 equiv). The reaction mixture was stirred for 2 hours. The crude was triturated in ACN (4 mL), then in Et₂O (3*4 mL), the solid was finally taken up in a water ACN mixture and freeze-dried to obtain Example 2 (38 mg, 49%) as a white solid.

¹H-NMR (DMSO-d₆, 400 MHz) δ: 1.74 (s, 6H, 2 CH₃); 1.81 (s, 9H, 3 CH₃); 2.92 (s, 3H, N—CH₃); 3.46-3.48 (m, 2H, CH₂); 3.65-3.67 (m, 2H, CH₂); 7.45 (d, J 8.3 Hz, 1H, Ar); 7.60-7.67 (m, 2H, Ar); 7.80-7.83 (m, 1H, Ar); 8.22 (s, 1H, Ar); 8.44 (d, J 8.3 Hz, 1H, Ar).

M/Z (M[³⁵Cl]+H)⁺: 471.3

Mp: 244-245° C.

### Example 3: methyl 2-(4-(1-(tert-butyl)-3-(4-chloro-3-fluorophenyl)-1H-pyrrolo[2,3-b]pyridine-6-carbonyl)-3,3-dimethyl-2-oxopiperazin-1-yl)acetate

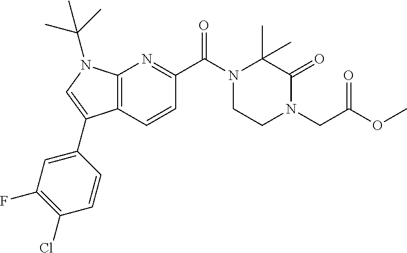 [SMILES: COC(=O)CN1CCN(C(=O)c2ccc3c(-c4ccc(Cl)c(F)c4)cn(C(C)(C)C)c3n2)C(C)(C)C1=O]

Example 3 was prepared according to general procedure (XIII) in DMA starting from Example 1 (408 mg) and methyl-2-bromoacetate (164 mg, 1.2 equiv). The reaction mixture was stirred for 20 hours. The crude was purified by flash chromatography (Interchim® 50 μm, CyHex 100% to CyHex/EtOAc 40:60) to obtain Example 3 (464 mg, 98%) as a yellow solid.

¹H-NMR (DMSO-d₆, 400 MHz) δ: 1.76 (s, 6H, 2 CH₃); 1.80 (s, 9H, 3 CH₃); 3.55-3.57 (m, 2H, CH₂); 3.67 (s, 3H, CH₃); 3.71-3.73 (m, 2H, CH₂); 4.19 (s, 2H, CH₂); 7.47 (d, J 8.3 Hz, 1H, Ar); 7.60-7.67 (m, 2H, Ar); 7.80-7.83 (m, 1H, Ar); 8.22 (s, 1H, Ar); 8.45 (d, J 8.3 Hz, 1H, Ar).

M/Z (M[³⁵Cl]+H)⁺: 529.2

Mp: 138-142° C.

### Example 4: 2-(4-(1-(tert-butyl)-3-(4-chloro-3-fluorophenyl)-1H-pyrrolo[2,3-b]pyridine-6-carbonyl)-3,3-dimethyl-2-oxopiperazin-1-yl)acetic acid

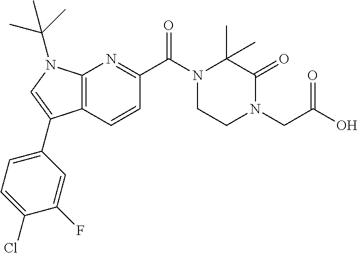 [SMILES: CC1(C)C(=O)N(CC(=O)O)CCN1C(=O)c1ccc2c(-c3ccc(Cl)c(F)c3)cn(C(C)(C)C)c2n1]

Example 4 was obtained (350 mg, 87%) as a white solid according to general procedure (IXa) starting from Example 3, (414 mg).

¹H-NMR (DMSO-d₆, 400 MHz) δ: 1.76 (s, 6H, 2 CH₃); 1.80 (s, 9H, 3 CH₃); 3.53-3.55 (m, 2H, CH₂); 3.70-3.72 (m, 2H, CH₂); 4.08 (s, 2H, CH₂); 7.46 (d, J 8.3 Hz, 1H, Ar); 7.60-7.67 (m, 2H, Ar); 7.80-7.83 (m, 1H, Ar); 8.22 (s, 1H, Ar); 8.44 (d, J 8.3 Hz, 1H, Ar); 12.74 (bs, 1H, COOH).

M/Z (M[³⁵Cl]+H)⁺: 515.2

Mp>250° C.

### Example 5: 2-(4-(1-(tert-butyl)-3-(4-chloro-3-fluorophenyl)-1H-pyrrolo[2,3-b]pyridine-6-carbonyl)-3,3-dimethyl-2-oxopiperazin-1-yl)acetamide

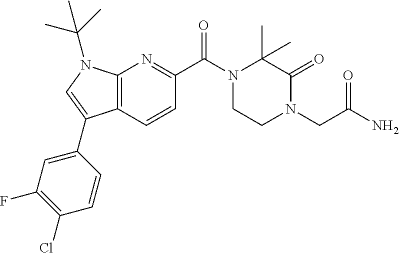 [SMILES: CC1(C)C(=O)N(CC(N)=O)CCN1C(=O)c1ccc2c(-c3ccc(Cl)c(F)c3)cn(C(C)(C)C)c2n1]

Example 5 was prepared according to general procedure (Xa) starting from Example 4 (75 mg) and ammonia in dioxane 0.5N (0.87 mL, 3 equiv), and using N,N-diisopropylethylamine (3.0 equiv). The crude was purified by preparative HPLC (40% to 80% ACN/H₂O), to obtain Example 5 (49 mg, 66%) as a white solid.

¹H-NMR (DMSO-d₆, 400 MHz) δ: 1.76 (s, 6H, 2 CH₃); 1.80 (s, 9H, 3 CH₃); 3.49-3.51 (m, 2H, CH₂); 3.70-3.71 (m, 2H, CH₂); 3.96 (s, 2H, CH₂); 7.02 (bs, 1H, NHₐH_{b}); 7.38 (bs, 1H, NHₐH_{b}); 7.45 (d, J 8.3 Hz, 1H, Ar); 7.60-7.67 (m, 2H, Ar); 7.80-7.83 (m, 1H, Ar); 8.22 (s, 1H, Ar); 8.44 (d, J 8.3 Hz, 1H, Ar).

M/Z (M[³⁵Cl]+H)⁺: 514.2.

Mp: 122-130° C.

### Example 6: 2-(4-(1-(tert-butyl)-3-(4-chloro-3-fluorophenyl)-1H-pyrrolo[2,3-b]pyridine-6-carbonyl)-3,3-dimethyl-2-oxopiperazin-1-yl)-N-methylacetamide

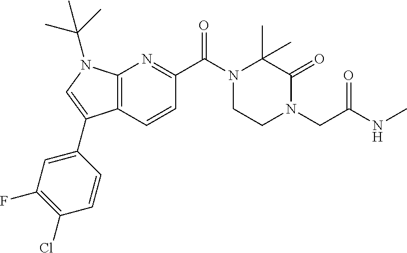 [SMILES: CNC(=O)CN1CCN(C(=O)c2ccc3c(-c4ccc(Cl)c(F)c4)cn(C(C)(C)C)c3n2)C(C)(C)C1=O]

Example 6 was prepared according to general procedure (Xa) starting from Example 4 (75 mg) and methylamine hydrochloride (11 mg, 1.1 equiv), and using N,N-diisopropylethylamine (4.0 equiv). The crude was purified by preparative HPLC (40% to 80% ACN/H₂O), to obtain Example 6 (54 mg, 70%) as a white solid.

¹H-NMR (DMSO-d₆, 400 MHz) δ: 1.76 (s, 6H, 2 CH₃); 1.80 (s, 9H, 3 CH₃); 2.60 (d, J 4.5 Hz, 2H, CH₂); 3.50-3.52 (m, 2H, CH₂); 3.70-3.72 (m, 2H, CH₂); 3.97 (s, 2H, CH₂); 7.45 (d, J 8.3 Hz, 1H, Ar); 7.60-7.67 (m, 2H, Ar); 7.80-7.86 (m, 2H, Ar+NH); 8.22 (s, 1H, Ar); 8.44 (d, J 8.3 Hz, 1H, Ar).

M/Z (M[³⁵Cl]+H)⁺: 528.3

Mp: 169-172° C.

### Example 7: 2-(4-(1-(tert-butyl)-3-(4-chloro-3-fluorophenyl)-1H-pyrrolo[2,3-b]pyridine-6-carbonyl)-3,3-dimethyl-2-oxopiperazin-1-yl)-N,N-dimethylacetamide

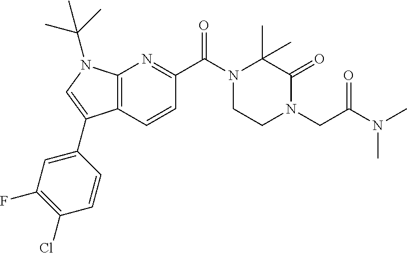 [SMILES: CN(C)C(=O)CN1CCN(C(=O)c2ccc3c(-c4ccc(Cl)c(F)c4)cn(C(C)(C)C)c3n2)C(C)(C)C1=O]

Example 7 was prepared according to general procedure (Xa) starting from Example 4 (75 mg) and dimethylamine hydrochloride (13 mg, 1.1 equiv), and using N,N-diisopropylethylamine (4.0 equiv). The crude was purified by preparative HPLC (40% to 80% ACN/H₂O), to obtain Example 7 (55 mg, 70%) as a white solid.

¹H-NMR (DMSO-d₆, 400 MHz) δ: 1.76 (s, 6H, 2 CH₃); 1.80 (s, 9H, 3 CH₃); 2.83 (s, 3H, N—CH₃); 2.95 (s, 3H, N—CH₃); 3.45-3.47 (m, 2H, CH₂); 3.70-3.73 (m, 2H, CH₂); 4.24 (s, 2H, CH₂); 7.45 (d, J 8.3 Hz, 1H, Ar); 7.60-7.67 (m, 2H, Ar); 7.80-7.83 (m, 1H, Ar); 8.22 (s, 1H, Ar); 8.44 (d, J 8.3 Hz, 1H, Ar).

M/Z (M[³⁵Cl]+H)⁺: 542.3

Mp: 96-103° C.

### Example 8: 4-(1-(tert-butyl)-3-(4-chloro-3-fluorophenyl)-1H-pyrrolo[2,3-b]pyridine-6-carbonyl)-3,3-dimethyl-1-(2-morpholino-2-oxoethyl)piperazin-2-one

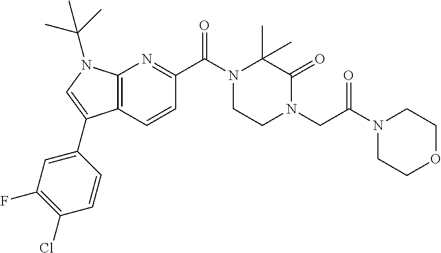 [SMILES: CC1(C)C(=O)N(CC(=O)N2CCOCC2)CCN1C(=O)c1ccc2c(-c3ccc(Cl)c(F)c3)cn(C(C)(C)C)c2n1]

Example 8 was prepared according to general procedure (Xa) starting from Example 4 (75 mg) and morpholine (14 mg, 1.1 equiv), and using N,N-diisopropylethylamine (3.0 equiv). The crude was purified by preparative HPLC (40% to 80% ACN/H₂O), to obtain Example 8 (63 mg, 74%) as a white solid.

¹H-NMR (DMSO-d₆, 400 MHz) δ: 1.76 (s, 6H, 2 CH₃); 1.80 (s, 9H, 3 CH₃); 3.41-3.48 (m, 6H, 3 CH₂); 3.56-3.59 (m, 4H, 2 CH₂); 3.71-3.73 (m, 2H, CH₂); 4.27 (s, 2H, CH₂); 7.46 (d, J 8.3 Hz, 1H, Ar); 7.60-7.67 (m, 2H, Ar); 7.80-7.83 (m, 1H, Ar); 8.22 (s, 1H, Ar); 8.44 (d, J 8.3 Hz, 1H, Ar).

M/Z (M[³⁵Cl]+H)⁺: 584.3

Mp: 110-118° C.

### Example 9: (1-(tert-butyl)-3-(4-chloro-3-fluorophenyl)-1H-pyrrolo[2,3-b]pyridin-6-yl)(6-oxa-2-azaspiro[3.4]octan-2-yl)methanone

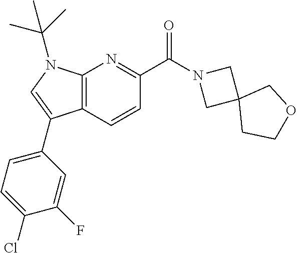 [SMILES: CC(C)(C)n1cc(-c2ccc(Cl)c(F)c2)c2ccc(C(=O)N3CC4(CCOC4)C3)nc21]

Example 9 was prepared according to general procedure (Xa) starting from Compound 78 (75 mg) and 6-oxa-2-azaspiro[3.4]octane hemioxalate (103 mg, 1.5 equiv), and using N,N-diisopropylethylamine (3.0 equiv). The crude was purified by flash chromatography (Interchim® 50 μm, CyHex/EtOAc 80:20 to CyHex/EtOAc 10:90). The residue was taken up in a water ACN mixture and freeze-dried to obtain Example 9 (90 mg, 94%) as a white solid.

¹H-NMR (DMSO-d₆, 400 MHz) δ: 1.82 (s, 9H, 3 CH₃); 2.19 (t, J 7.0 Hz, 2H, O—CH₂—CH₂); 3.74 (t, J 7.0 Hz, 2H, O—CH₂—CH₂); 3.80-3.88 (m, 2H, O—CH₂—C); 4.08-4.14 (m, 2H, N—CH₂); 4.69-4.74 (m, 2H, N—CH₂); 7.60-7.67 (m, 2H, Ar); 7.79-7.84 (m, 1H, Ar); 7.87 (d, J 8.3 Hz, 1H, Ar); 8.25 (s, 1H, Ar); 8.44 (d, J 8.3 Hz, 1H, Ar).

M/Z (M[³⁵Cl]+H)⁺: 442.2

Mp: 105-110° C.

### Example 10: (1-(tert-butyl)-3-(4-chloro-3-fluorophenyl)-1H-pyrrolo[2,3-b]pyridin-6-yl)(2-oxa-6-azaspiro[3.3]heptan-6-yl)methanone

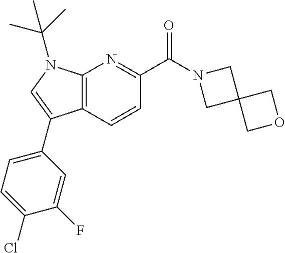 [SMILES: CC(C)(C)n1cc(-c2ccc(Cl)c(F)c2)c2ccc(C(=O)N3CC4(COC4)C3)nc21]

Example 10 was prepared according to general procedure (Xa) starting from Compound 78 (75 mg) and 2-Oxa-6-aza-spiro[3.3]heptane (26 mg, 1.2 equiv), and using N,N-diisopropylethylamine (3.0 equiv). The crude was purified by preparative HPLC (40% to 80% ACN/H₂O), to obtain Example 10 (70 mg, 76%) as a white solid.

¹H-NMR (DMSO-d₆, 400 MHz) δ: 1.85 (s, 9H, 3 CH₃); 4.29 (s, 2H, CH₂); 4.74 (s, 4H, 2 CH₂); 4.91 (s, 2H, CH₂); 7.60-7.67 (m, 2H, Ar); 7.79-7.84 (m, 1H, Ar); 7.87 (d, J 8.4 Hz, 1H, Ar); 8.26 (s, 1H, Ar); 8.44 (d, J 8.4 Hz, 1H, Ar).

M/Z (M[³⁵Cl]+H)⁺: 428.2

Mp: 95-100° C.

### Example 11: (1-(tert-butyl)-3-(4-chloro-3-fluorophenyl)-1H-pyrrolo[2,3-b]pyridin-6-yl)((1R,3r,5S)-3-hydroxy-8-azabicyclo[3.2.1]octan-8-yl)methanone

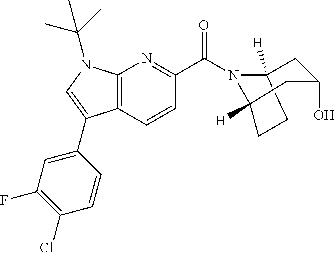 [SMILES: CC(C)(C)n1cc(-c2ccc(Cl)c(F)c2)c2ccc(C(=O)N3[C@@H]4CC[C@H]3CC(O)C4)nc21]

Example 11 was prepared according to general procedure (Xa) starting from Compound 78 (75 mg) and (1R,3r,5S)-8-azabicyclo[3.2.1]octan-3-ol (33 mg, 1.2 equiv), and using N,N-diisopropylethylamine (3.0 equiv). The crude was purified by flash chromatography (Interchim® 50 μm, CyHex 100% to CyHex/EtOAc 10:90). The residue was taken up in a water ACN mixture and freeze-dried to obtain Example 11 (86 mg, 87%) as a beige solid.

¹H-NMR (DMSO-d₆, 400 MHz) δ: 1.72-1.92 (m, 13H, 3 CH₃+2 CH₂); 2.03-2.10 (m, 1H, CHₐH_{b}); 2.13-2.20 (m, 1H, CHₐH_{b}); 2.22-2.30 (m, 2H, CH₂); 4.00-4.05 (m, 1H, CH); 4.64-4.66 (m, 1H, OH); 4.66-4.71 (m, 1H, CH); 4.74-4.79 (m, 1H, CH); 7.58 (d, J 8.3 Hz, 1H, Ar); 7.59-7.67 (m, 2H, Ar); 7.79-7.84 (m, 1H, Ar); 8.21 (s, 1H, Ar); 8.41 (d, J 8.3 Hz, 1H, Ar).

M/Z (M[³⁵Cl]+H)⁺: 456.3

Mp: 118-126° C.

### Example 12: (1-(tert-butyl)-3-(4-chloro-3-fluorophenyl)-1H-pyrrolo[2,3-b]pyridin-6-yl)((1R,3s,5S)-3-hydroxy-8-azabicyclo[3.2.1]octan-8-yl)methanone

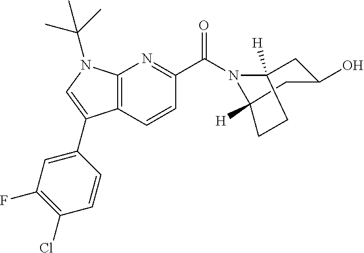 [SMILES: CC(C)(C)n1cc(-c2ccc(Cl)c(F)c2)c2ccc(C(=O)N3[C@@H]4CC[C@H]3CC(O)C4)nc21]

Example 12 was prepared according to general procedure (Xa) starting from Compound 78 (75 mg) and Exo-3-hydroxy-8-azabicyclo[3.2.1]octane hydrochloride (35 mg, 1.0 equiv), and using N,N-diisopropylethylamine (3.0 equiv). The crude was purified by flash chromatography (Interchim® 50 μm, CyHex 100% to EtOAc 100%). The residue was taken up in a water ACN mixture and freeze-dried to obtain Example 12 (79 mg, 80%) as a white solid.

¹H-NMR (DMSO-d₆, 400 MHz) δ: 1.52-1.61 (m, 1H, CHₐH_{b}); 1.64-1.77 (m, 3H, CHₐH_{b}+CH₂); 1.81 (s, 9H, 3 CH₃); 1.85-1.98 (m, 4H, 2 CH₂); 3.96-4.07 (m, 1H, CH—OH); 4.63 (d, J 5.8 Hz, 1H, CH—OH), 4.70-4.75 (m, 1H, CHₐH_{b}); 4.78-4.84 (m, 1H, CHₐH_{b}); 7.59 (d, J 8.4 Hz, 1H, Ar); 7.59-7.67 (m, 2H, Ar); 7.79-7.85 (m, 1H, Ar); 8.22 (s, 1H, Ar); 8.42 (d, J 8.4 Hz, 1H, Ar).

M/Z (M[³⁵Cl]+H)⁺: 456.3

Mp: 95-100° C.

### Example 13: 8-(1-(tert-butyl)-3-(4-chloro-3-fluorophenyl)-1H-pyrrolo[2,3-b]pyridine-6-carbonyl)-2,8-diazaspiro[4.5]decan-1-one

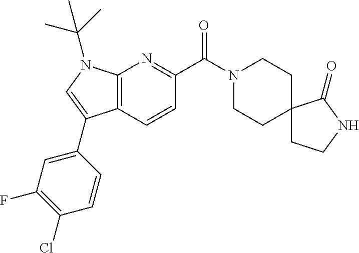 [SMILES: CC(C)(C)n1cc(-c2ccc(Cl)c(F)c2)c2ccc(C(=O)N3CCC4(CCNC4=O)CC3)nc21]

Example 13 was prepared according to general procedure (Xa) starting from Compound 78 (75 mg) and 2,8-diazaspiro[4.5]decan-1-one (37 mg, 1.1 equiv), and using N,N-diisopropylethylamine (3.0 equiv). The crude was triturated in ACN (15 mL), then in Et₂O (2*10 mL) to obtain Example 13 (50 mg, 48%) as a white solid.

¹H-NMR (DMSO-d₆, 400 MHz) δ: 1.36-1.39 (m, 1H, CHₐH_{b}); 1.49-1.52 (m, 1H, CHₐH_{b}); 1.68-1.76 (m, 2H, CH₂); 1.80 (s, 9H, 3 CH₃); 1.99-2.07 (m, 2H, CH₂); 3.09-3.27 (m, 4H, 2CH₂); 3.99-4.02 (m, 1H, CHₐH_{b}); 4.36-4.39 (m, 1H, CHₐH_{b}); 7.42 (d, J 8.3 Hz, 1H, Ar); 7.61-7.66 (m, 3H, Ar+CONH); 7.80-7.83 (m, 1H, Ar); 8.20 (s, 1H, Ar); 8.43 (d, J 8.3 Hz, 1H, Ar).

M/Z (M[³⁵Cl]+H)⁺: 483.2

Mp>250° C.

### Example 14: 8-(1-(tert-butyl)-3-(4-chloro-3-fluorophenyl)-1H-pyrrolo[2,3-b]pyridine-6-carbonyl)-2,8-diazaspiro[4.5]decan-3-one

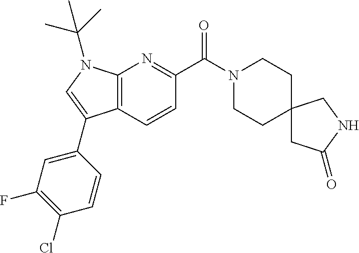 [SMILES: CC(C)(C)n1cc(-c2ccc(Cl)c(F)c2)c2ccc(C(=O)N3CCC4(CC3)CNC(=O)C4)nc21]

Example 14 was prepared according to general procedure (Xa) starting from Compound 78 (50 mg) and 2,8-diazaspiro[4.5]decan-3-one (37 mg, 1.1 equiv), and using N,N-diisopropylethylamine (3.0 equiv). The crude was triturated in ACN (15 mL), then in Et₂O (2*10 mL) and then H₂O (3*10 mL) to obtain Example 14 (63 mg, 60%) as a white solid.

¹H-NMR (DMSO-d₆, 400 MHz) δ: 1.59-1.65 (m, 4H, 2 CH₂); 1.80 (s, 9H, 3 CH₃); 2.09-2.19 (m, 2H, CH₂); 3.07-3.15 (m, 2H, CH₂); 3.49-3.65 (m, 3H, CH₂+CHₐH_{b}); 3.78-3.83 (m, 1H, CHₐH_{b}); 7.41 (d, J 8.3 Hz, 1H, Ar); 7.54 (s, 1H, CONH); 7.60-7.66 (m, 2H, Ar); 7.80-7.83 (m, 1H, Ar); 8.20 (s, 1H, Ar); 8.42 (d, J 8.3 Hz, 1H, Ar).

M/Z (M[³⁵Cl]+H)⁺: 483.3

Mp>250° C.

### Example 15: (3aS,6aS)-1-(1-(tert-butyl)-3-(4-chloro-3-fluorophenyl)-1H-pyrrolo[2,3-b]pyridine-6-carbonyl)hexahydropyrrolo[3,4-b]pyrrol-6(1H)-one

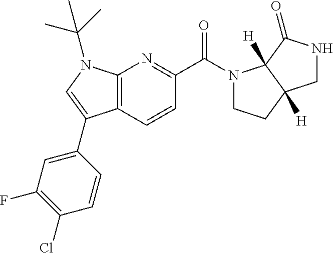 [SMILES: CC(C)(C)n1cc(-c2ccc(Cl)c(F)c2)c2ccc(C(=O)N3CC[C@H]4CNC(=O)[C@H]43)nc21]

Example 15 was prepared according to general procedure (Xa) starting from Compound 78 (75 mg) and (3aS,6aS)-hexahydropyrrolo[3,4-b]pyrrol-6(1H)-one (33 mg, 1.2 equiv), and using N,N-diisopropylethylamine (3.0 equiv). The crude was purified by preparative HPLC (30% to 70% ACN/H₂O), to obtain Example 15 (70 mg, 71%), as a white solid.

¹H-NMR (DMSO-d₆, 400 MHz) δ: 1.62-1.72 (m, 0.8H, one rotamer of CH₂); 1.78 (s, 5.6H, one rotamer of N—C—CH₃); 1.81 (s, 3.4H, one rotamer of N—C—CH₃); 2.12-2.20 (m, 0.4H, one rotamer of CH₂); 2.20-2.30 (m, 0.8H, one rotamer of CH₂); 2.93-3.01 (m, 0.4H, one rotamer of CH₂); 3.02-3.08 (m, 1H, CH); 3.13-3.23 (m, 0.7H, one rotamer of CH₂); 3.38-3.45 (m, 0.6H, one rotamer of CH₂); 3.47-3.53 (m, 0.4H, one rotamer of CH₂); 3.55-3.64 (m, 0.6H, one rotamer of CH₂); 3.65-3.75 (m, 0.6H, one rotamer of CH₂); 3.82-3.90 (m, 0.7H, one rotamer of CH₂); 5.09 (d, J 8.3 Hz, 0.4H, one rotamer of CH); 5.73 (d, J 8.3 Hz, 0.4H, one rotamer of CH); 7.58-7.72 (m, 3H, Ar); 7.75-7.85 (m, 2H, Ar); 8.19 (s, 0.6H, one rotamer of Ar); 8.24 (s, 0.4H, one rotamer of Ar); 8.40 (d, J 8.3 Hz, 0.6H, one rotamer of Ar); 8.44 (d, J 8.3 Hz, 0.4H, one rotamer of Ar).

M/Z (M[³⁵Cl]+H)⁺: 455.2

Mp: 127-135° C.

### Example 16: (1-(tert-butyl)-3-(4-chloro-3-fluorophenyl)-1H-pyrrolo[2,3-b]pyridin-6-yl)(2,2-dimethylpyrrolidin-1-yl)methanone

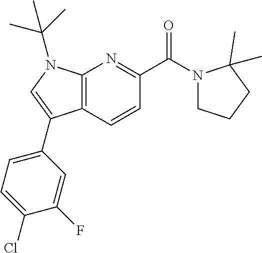 [SMILES: CC1(C)CCCN1C(=O)c1ccc2c(-c3ccc(Cl)c(F)c3)cn(C(C)(C)C)c2n1]

Example 16 was prepared according to general procedure (Xa) starting from Compound 78 (60 mg) and 2,2-dimethyl-Pyrrolidine (21 mg, 1.2 equiv), and using N,N-diisopropylethylamine (3.0 equiv). The crude was purified by preparative HPLC (60% to 100% ACN/H₂O) to obtain Example 16 (59 mg, 80%) as a white solid.

¹H-NMR (DMSO-d₆, 400 MHz) δ: 1.54 (s, 6H, 2N—C—CH₃); 1.80 (s, 9H, 3N—C—CH₃); 1.80-1.88 (m, 4H, 2 CH₂); 3.70 (t, J 6.4 Hz, 2H, N—CH₂); 7.49 (d, J 8.2 Hz, 1H, Ar); 7.59-7.67 (m, 2H, Ar); 7.78-7.83 (m, 1H, Ar); 8.19 (s, 1H, Ar); 8.40 (d, J 8.2 Hz, 1H, Ar).

M/Z (M[³⁵Cl]+H)⁺: 428.1

Mp: 65-69° C.

### Example 17: 4-(1-(tert-butyl)-3-(4-chloro-3-fluorophenyl)-1H-pyrrolo[2,3-b]pyridine-6-carbonyl)-1,4-diazepan-2-one

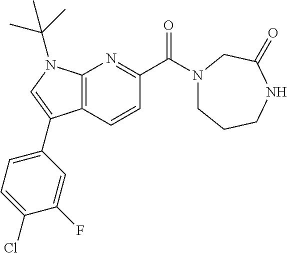 [SMILES: CC(C)(C)n1cc(-c2ccc(Cl)c(F)c2)c2ccc(C(=O)N3CCCNC(=O)C3)nc21]

Example 17 was prepared according to general procedure (Xa) starting from Compound 78 (60 mg) and hexahydro-2H-1,4-diazepin-2-one (24 mg, 1.2 equiv), and using N,N-diisopropylethylamine (3.0 equiv). The crude was purified by preparative HPLC (40% to 80% ACN/H₂O) to obtain Example 17 (39 mg, 51%), as a white solid.

¹H-NMR (DMSO-d₆, 80° C., 400 MHz) δ: 1.83 (s, 9H, 3 CH₃); 1.84-1.94 (m, 2H, CH₂); 3.24-3.30 (m, 2H, N—CH₂); 3.75-3.83 (m, 2H, N—CH₂); 4.33 (s, 2H, N—CH₂); 7.29 (m, 1H, NH); 7.41 (d, J 8.2 Hz, 1H, Ar); 7.56-7.63 (m, 2H, Ar); 7.70-7.75 (m, 1H, Ar); 8.12 (s, 1H, Ar); 8.37 (d, J 8.2 Hz, 1H, Ar).

M/Z (M[³⁵Cl]+H)⁺: 443.2

Mp: 118-123° C.

### Example 18: 1-(1-(tertbutyl)-3-(4-chloro-3-fluorophenyl)-1H-pyrrolo[2,3-b]pyridine-6-carbonyl)-1,4-diazepan-5-one

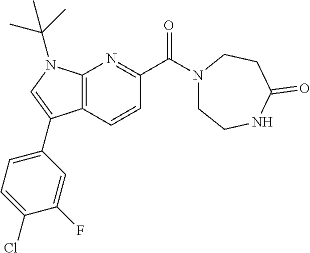 [SMILES: CC(C)(C)n1cc(-c2ccc(Cl)c(F)c2)c2ccc(C(=O)N3CCNC(=O)CC3)nc21]

Example 18 was prepared according to general procedure (Xa) starting from Compound 78 (60 mg) and 2,3,6,7-Tetrahydro-(1H)-1,4-diazepin-5(4H)-one (24 mg, 1.2 equiv), and using N,N-diisopropylethylamine (3.0 equiv). The crude was purified by flash chromatography (Interchim® 50 μm, DCM 100% to DCM/MeOH 96:4). The beige solid was triturated in 2 mL of ACN to obtain Example 18 (41 mg, 54%) as a white solid.

¹H-NMR (DMSO-d₆, 80° C., 400 MHz) δ: 1.83 (s, 9H, 3 CH₃); 2.56-2.62 (m, 2H, CH₂); 3.25-3.34 (m, 2H, N—CH₂); 3.70-3.88 (m, 4H, 2N—CH₂); 7.37 (bs, 1H, NH); 7.44 (d, J 8.2 Hz, 1H, Ar); 7.56-7.64 (m, 2H, Ar); 7.71-7.76 (m, 1H, Ar); 8.13 (s, 1H, Ar); 8.38 (d, J 8.2 Hz, 1H, Ar).

M/Z (M[³⁵Cl]+H)⁺: 443.2

Mp: 165-171° C.

### Example 19: ((1R,5S)-3-oxa-8-azabicyclo[3.2.1]octan-8-yl)(1-(tertbutyl)-3-(4-chloro-3-fluorophenyl)-1H-pyrrolo[2,3-b]pyridin-6-yl)methanone

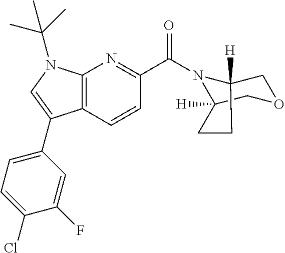 [SMILES: CC(C)(C)n1cc(-c2ccc(Cl)c(F)c2)c2ccc(C(=O)N3[C@@H]4CC[C@H]3COC4)nc21]

Example 19 was prepared according to general procedure (Xa) starting from Compound 78 (60 mg) and 3-Oxa-8-azabicyclo[3.2.1]octane Hydrochloride (31 mg, 1.2 equiv), and using N,N-diisopropylethylamine (4.0 equiv). The crude was purified by flash chromatography (Interchim® 50 μm, CyHex 100% to CyHex/EtOAc 20:80) to obtain Example 19 (42 mg, 55%) as a white solid.

¹H-NMR (DMSO-d₆, 400 MHz) δ: 1.80 (s, 9H, 3 CH₃); 1.88-2.00 (m, 4H, 2N—CH—CH₂); 3.62-3.67 (m, 1H, N—CH); 3.70 (s, 2H, CH₂); 3.80-3.86 (m, 1H, N—CH); 4.63-4.68 (m, 1H, N—CH); 4.73-4.78 (m, 1H, N—CH); 7.59-7.68 (m, 3H, Ar); 7.78-7.84 (m, 1H, Ar); 8.23 (s, 1H, Ar); 8.44 (d, J 8.2 Hz, 1H, Ar).

M/Z (M[³⁵Cl]+H)⁺: 442.2

Mp: 156-160° C.

### Example 20: 4-(1-(tertbutyl)-3-(4-chloro-3-fluorophenyl)-1H-pyrrolo[2,3-b]pyridine-6-carbonyl)-1-methylpiperazin-2-one

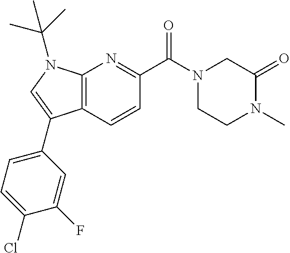 [SMILES: CN1CCN(C(=O)c2ccc3c(-c4ccc(Cl)c(F)c4)cn(C(C)(C)C)c3n2)CC1=O]

Example 20 was prepared according to general procedure (Xa) starting from Compound 78 (60 mg) and 1-Methyl-2-piperazinone (24 mg, 1.2 equiv), and using N,N-diisopropylethylamine (3.0 equiv). The crude was purified by flash chromatography (Interchim® 50 μm, DCM 100% to DCM/MeOH 97:3). The resulting beige solid was triturated in ACN (2 mL) to obtain Example 20 (39 mg, 51%) as a white solid.

¹H-NMR (DMSO-d₆, 80° C., 400 MHz) δ: 1.84 (s, 9H, 3 CH₃); 2.92 (s, 3H, N—CH₃); 3.46 (t, J 5.5 Hz, 2H, N—CH₂—CH₂—N); 3.97 (t, J 5.5 Hz, 2H, N—CH₂—CH₂—N); 4.30 (bs, 2H, N—CH₂—CO); 7.54 (d, J 8.2 Hz, 1H, Ar); 7.57-7.63 (m, 2H, Ar); 7.73-7.77 (m, 1H, Ar); 8.16 (s, 1H, Ar); 8.41 (d, J 8.2 Hz, 1H, Ar).

M/Z (M[³⁵Cl]+H)⁺: 443.1

Mp: 170-173° C.

### Example 21: methyl 1-(1-(tert-butyl)-3-(4-chloro-3-fluorophenyl)-1H-pyrrolo[2,3-b]pyridine-6-carbonyl)-3,3-dimethylpiperidine-4-carboxylate

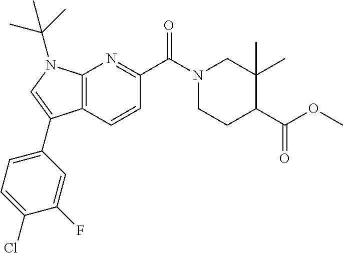 [SMILES: COC(=O)C1CCN(C(=O)c2ccc3c(-c4ccc(Cl)c(F)c4)cn(C(C)(C)C)c3n2)CC1(C)C]

Example 21 was prepared according to general procedure (Xa) starting from Compound 78 (170 mg) and methyl 3,3-dimethylpiperidine-4-carboxylate hydrochloride (122 mg, 1.2 equiv), and using N,N-diisopropylethylamine (4.0 equiv). The crude was purified by flash chromatography (Interchim® 50 μm, CyHex 100% to CyHex/EtOAc 80:20) to obtain Example 21 (191 mg, 78%) as a colorless waxy solid.

¹H-NMR (DMSO-d₆, 400 MHz) δ: 0.70 (s, 1H, one rotamer of CH₃); 0.80 (s, 1H, one rotamer of CH₃); 0.98 (s, 2H, one rotamer of CH₃); 1.07 (s, 2H, one rotamer of CH₃); 1.60-1.68 (m, 0.7H, one rotamer of N—CH₂—CH₂); 1.70-1.90 (m, 9.6H, 3 CH₃+one rotamer of NCH₂—CH₂); 1.95-2.06 (m, 0.7H, N—CH₂—CH₂); 2.54-2.58 (m, 0.7H, one rotamer of N—CH₂+one rotamer of CH); 2.78-2.85 (m, 0.7H, one rotamer of N—CH₂+one rotamer of CH); 2.95-3.05 (m, 0.6H, one rotamer of N—CH₂); 3.08-3.20 (m, 1H, one rotamer of N—CH₂); 3.60 (s, 1H, one rotamer of O—CH₃); 3.63 (s, 2H, one rotamer of O—CH₃); 3.63-3.68 (m, 0.4H, one rotamer of N—CH₂); 3.90-4.00 (m, 0.6H, one rotamer of N—CH₂); 4.10-4.17 (m, 0.6H, one rotamer of N—CH₂); 4.43-4.52 (m, 0.4H, one rotamer of N—CH₂); 7.39 (d, J 8.2 Hz, 0.4H, one rotamer of Ar); 7.41 (d, J 8.2 Hz, 0.6H, one rotamer of Ar); 7.59-7.68 (m, 2H, Ar); 7.78-7.84 (m, 1H, Ar); 8.19 (s, 0.4H, one rotamer of Ar); 8.20 (s, 0.6H, one rotamer of Ar); 8.41 (d, J 8.2 Hz, 0.4H, one rotamer of Ar); 8.43 (d, J 8.2 Hz, 0.6H, one rotamer of Ar).

M/Z (M[³⁵Cl]+H)⁺: 500.2

Mp: 157-160° C.

### Example 22: 1-(1-(tert-butyl)-3-(4-chloro-3-fluorophenyl)-1H-pyrrolo[2,3-b]pyridine-6-carbonyl)-3,3-dimethylpiperidine-4-carboxylic acid

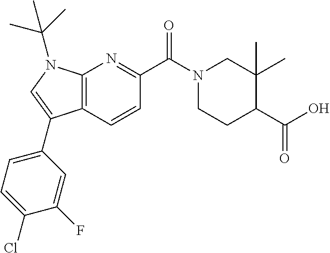 [SMILES: CC1(C)CN(C(=O)c2ccc3c(-c4ccc(Cl)c(F)c4)cn(C(C)(C)C)c3n2)CCC1C(=O)O]

Example 22 was prepared according to general procedure (IXa) starting from Example 21, (185 mg). The crude was purified by flash chromatography (Interchim® 50 μm, DCM 100% to DCM/MeOH 96:4) to obtain Example 22 (145 mg, 81%) as a white solid.

¹H-NMR (DMSO-d₆, 400 MHz) δ: 0.73 (s, 1H, one rotamer of CH₃); 0.83 (s, 1H, one rotamer of CH₃); 1.01 (s, 2H, one rotamer of CH₃); 1.10 (s, 2H, one rotamer of CH₃); 1.60-1.68 (m, 0.7H, one rotamer of N—CH₂—CH₂); 1.70-1.90 (m, 9.6H, 3 CH₃+one rotamer of N—CH₂—CH₂); 1.95-2.06 (m, 0.7H, N—CH₂—CH₂); 2.34-2.44 (m, 1H, CH); 2.75-2.81 (m, 0.6H, one rotamer of N—CH₂); 2.94-3.04 (m, 0.4H, one rotamer of N—CH₂); 3.06-3.16 (m, 1H, one rotamer of N—CH₂); 3.60-3.68 (m, 0.4H, one rotamer of N—CH₂); 3.90-4.00 (m, 0.6H, one rotamer of N—CH₂); 4.10-4.17 (m, 0.6H, one rotamer of N—CH₂); 4.43-4.52 (m, 0.4H, one rotamer of N—CH₂); 7.39 (d, J 8.2 Hz, 0.4H, one rotamer of Ar); 7.41 (d, J 8.2 Hz, 0.6H, one rotamer of Ar); 7.59-7.68 (m, 2H, Ar); 7.78-7.84 (m, 1H, Ar); 8.19 (s, 0.4H, one rotamer of Ar); 8.20 (s, 0.6H, one rotamer of Ar); 8.41 (d, J 8.2 Hz, 0.4H, one rotamer of Ar); 8.43 (d, J 8.2 Hz, 0.6H, one rotamer of Ar); 12.20 (s, 1H, COOH).

M/Z (M[³⁵Cl]+H)⁺: 486.2

Mp: 130-140° C.

### Example 23: 1-(1-(tert-butyl)-3-(4-chloro-3-fluorophenyl)-1H-pyrrolo[2,3-b]pyridine-6-carbonyl)-3,3-dimethylpiperidine-4-carboxamide

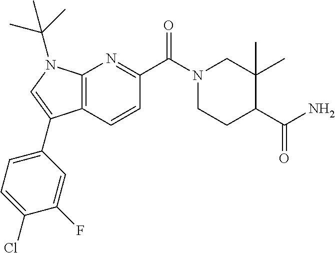 [SMILES: CC1(C)CN(C(=O)c2ccc3c(-c4ccc(Cl)c(F)c4)cn(C(C)(C)C)c3n2)CCC1C(N)=O]

Example 23 was prepared according to general procedure (Xa) starting from Example 22, (110 mg) and ammonia 0.5M in dioxane (1.36 mL, 3.0 equiv), and using N,N-diisopropylethylamine (3.0 equiv). The crude was purified by flash chromatography (Interchim® 50 μm, DCM 100% to DCM/MeOH 96:4) to obtain Example 23 (70 mg, 64%) as a white solid.

¹H-NMR (DMSO-d₆, 400 MHz) δ: 0.74 (s, 1H, one rotamer of CH₃); 0.78 (s, 1H, one rotamer of CH₃); 1.02 (s, 2H, one rotamer of CH₃); 1.05 (s, 2H, one rotamer of CH₃); 1.48-1.57 (m, 0.6H, one rotamer of N—CH₂—CH₂); 1.58-1.66 (m, 0.4H, one rotamer of N—CH₂—CH₂); 1.75-1.84 (m, 9H, 3 CH₃); 1.86-1.98 (m, 1H, one rotamer of N—CH₂—CH₂); 2.16-2.26 (m, 1H, CH); 2.78-2.85 (m, 0.6H, one rotamer of N—CH₂); 2.99-3.05 (m, 0.4H, one rotamer of N—CH₂); 3.08-3.20 (m, 1H, one rotamer of N—CH₂); 3.60-3.70 (m, 0.4H, one rotamer of N—CH₂); 3.90-4.00 (m, 0.6H, one rotamer of N—CH₂); 4.05-4.12 (m, 0.6H, one rotamer of NCH₂); 4.36-4.45 (m, 0.4H, one rotamer of N—CH₂); 6.78 (bs, 1H, NH); 7.20-7.28 (m, 1H, NH); 7.37 (d, J 8.2 Hz, 0.4H, one rotamer of Ar); 7.39 (d, J 8.2 Hz, 0.6H, one rotamer of Ar); 7.58-7.66 (m, 2H, Ar); 7.78-7.84 (m, 1H, Ar); 8.19 (s, 0.4H, one rotamer of Ar); 8.20 (s, 0.6H, one rotamer of Ar); 8.41 (d, J 8.2 Hz, 0.4H, one rotamer of Ar); 8.42 (d, J 8.2 Hz, 0.6H, one rotamer of Ar).

M/Z (M[³⁵Cl]+H)⁺: 485.2

Mp: 119-120° C.

### Example 24: 8-(1-(tert-butyl)-3-(4-chloro-3-fluorophenyl)-1H-pyrrolo[2,3-b]pyridine-6-carbonyl)-1,3,8-triazaspiro[4.5]decane-2,4-dione

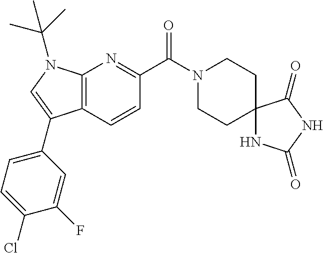 [SMILES: CC(C)(C)n1cc(-c2ccc(Cl)c(F)c2)c2ccc(C(=O)N3CCC4(CC3)NC(=O)NC4=O)nc21]

Example 24 was prepared according to general procedure (Xa) starting from Compound 78 (60 mg) and 2,4-Dioxo-1,3,8-triazaspiro[4.5]decane (41 mg, 1.2 equiv), and using N,N-diisopropylethylamine (3.0 equiv). The crude was purified by flash chromatography (Interchim® 50 μm, CyHex/EtOAc 30:70 to EtOAc 100%) to obtain Example 24 (27 mg, 27%) as a white solid.

¹H-NMR (DMSO-d₆, 400 MHz) δ: 1.56-1.65 (m, 1H, N—CH₂—CHₐH_{b}); 1.68-1.76 (m, 1H, N—CH₂—CHₐH_{b}); 1.80 (s, 9H, 3 CH₃); 1.83-1.95 (m, 2H, N—CH₂—CH₂); 3.31-3.38 (m, 1H, N—CHₐH_{b}); 3.44-3.54 (m, 1H, N—CHₐH_{b}); 3.94-4.03 (m, 1H, N—CHₐH_{b}); 4.31-4.39 (m, 1H, NCHₐH_{b}); 7.45 (d, J 8.2 Hz, 1H, Ar); 7.59-7.67 (m, 2H, Ar); 7.79-7.84 (m, 1H, Ar); 8.21 (s, 1H, Ar); 8.44 (d, J 8.2 Hz, 1H, Ar); 8.60 (bs, 1H, NH); 10.74 (bs, 1H, NH).

M/Z (M[³⁵Cl]+H)⁺: 498.2

Mp: 172-178° C.

### Example 25: methyl 6-(4-(1-(tert-butyl)-3-(4-chloro-3-fluorophenyl)-1H-pyrrolo[2,3-b]pyridine-6-carbonyl)-3,3-dimethylpiperazin-1-yl)-2,4-dimethylnicotinate

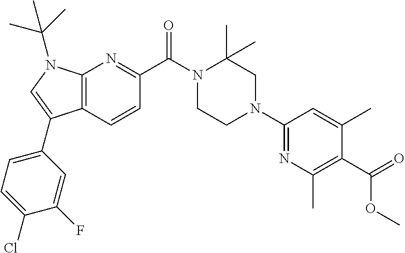 [SMILES: COC(=O)c1c(C)cc(N2CCN(C(=O)c3ccc4c(-c5ccc(Cl)c(F)c5)cn(C(C)(C)C)c4n3)C(C)(C)C2)nc1C]

Example 25 was prepared according to general procedure (Xa) starting from Compound 78 (400 mg) and Compound 95 (398 mg, 1.1 equiv), and using N,N-diisopropylethylamine (4.0 equiv). The crude was purified by flash chromatography (Interchim® 50 μm, CyHex 100% to CyHex/EtOAc 80:20) to obtain Example 25 (569 mg, 81%) as a beige solid.

M/Z (M[³⁵Cl]+H)⁺: 606.4

### Example 26: 6-(4-(1-(tert-butyl)-3-(4-chloro-3-fluorophenyl)-1H-pyrrolo[2,3-b]pyridine-6-carbonyl)-3,3-dimethylpiperazin-1-yl)-2,4-dimethylnicotinic acid

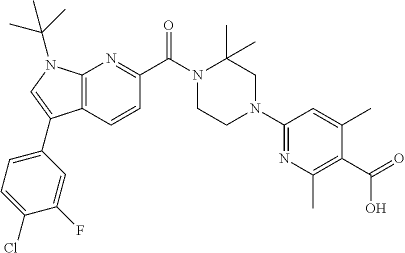 [SMILES: Cc1cc(N2CCN(C(=O)c3ccc4c(-c5ccc(Cl)c(F)c5)cn(C(C)(C)C)c4n3)C(C)(C)C2)nc(C)c1C(=O)O]

Example 26 was prepared according to general procedure (XI) starting from Example 25 (565 mg). The crude was purified by preparative HPLC (30% to 70% ACN/H₂O), to obtain Example 26 (280 mg, 51%) as a white solid.

¹H-NMR (DMSO-d₆, 400 MHz) δ: 1.55 (s, 6H, 2N—C—CH₃); 1.82 (s, 9H, 3N—C—CH₃); 2.27 (s, 3H, Pyr-CH₃); 2.38 (s, 3H, Pyr-CH₃); 3.52-3.58 (m, 2H, N—CH₂—CH₂—N); 3.82-3.88 (m, 2H, N—CH₂—CH₂—N); 3.92 (s, 2H, N—CH₂); 6.31 (s, 1H, Ar); 7.41 (d, J 8.2 Hz, 1H, Ar); 7.60-7.68 (m, 2H, Ar); 7.79-7.84 (m, 1H, Ar); 8.21 (s, 1H, Ar); 8.43 (d, J 8.2 Hz, 1H, Ar); COOH signal not observed.

M/Z (M[³⁵Cl]+H)⁺: 592.3

Mp: 147-156° C.

### Example 27: 4-(3-(4-chloro-3-fluorophenyl)-1-(4-methoxybenzyl)-1H-pyrrolo[2,3-b]pyridine-6-carbonyl)-3,3-dimethylpiperazin-2-one

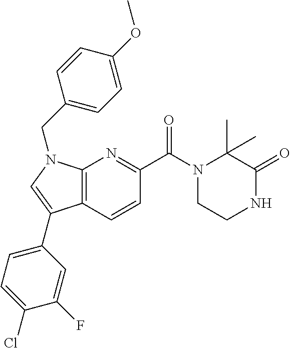 [SMILES: COc1ccc(Cn2cc(-c3ccc(Cl)c(F)c3)c3ccc(C(=O)N4CCNC(=O)C4(C)C)nc32)cc1]

Example 27 was prepared according to general procedure (Xa) starting from Compound 79 (80 mg) and 3,3-dimethylpiperazin-2-one (37 mg, 1.5 equiv), and using N,N-diisopropylethylamine (3.0 equiv). The crude was triturated in ACN (10 mL) then in Et₂O (10 mL) to obtain Example 27 (77 mg, 76%) as a white solid.

¹H-NMR (DMSO-d₆, 400 MHz) δ: 1.72 (s, 6H, 2 CH₃); 3.17-3.23 (m, 2H, N—CH₂—CH₂—N); 3.43-3.49 (m, 2H, N—CH₂—CH₂—N); 3.70 (s, 3HO—CH₃); 5.45 (s, 2H, N—CH₂-Ph); 6.85-6.90 (m, 2H, Ar); 7.21-7.26 (m, 2H, Ar); 7.46 (d, J 8.2 Hz, 1H, Ar); 7.59-7.66 (m, 2H, Ar); 7.73-7.78 (m, 1H, Ar); 8.04-8.09 (m, 1H, NH); 8.33 (s, 1H, Ar); 8.48 (d, J 8.2 Hz, 1H, Ar).

M/Z (M[³⁵Cl]+H)⁺: 521.2

Mp>250° C.

### Example 28: 4-(3-(4-chloro-3-fluorophenyl)-1-(3-methoxycyclobutyl)-1H-pyrrolo[2,3-b]pyridine-6-carbonyl)-3,3-dimethylpiperazin-2-one

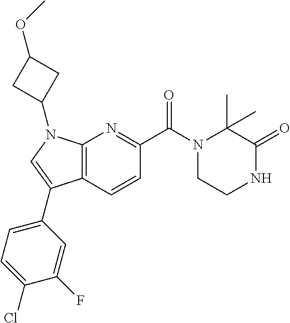 [SMILES: COC1CC(n2cc(-c3ccc(Cl)c(F)c3)c3ccc(C(=O)N4CCNC(=O)C4(C)C)nc32)C1]

Example 28 was prepared according to general procedure (Xa) starting from Compound 80 (31 mg) and 3,3-dimethylpiperazin-2-one (16 mg, 1.5 equiv), and using N,N-diisopropylethylamine (3.0 equiv). The crude was purified by preparative HPLC (30% to 70% ACN/H₂O) to obtain Example 258 (24 mg, 60%) as a white solid.

¹H-NMR (DMSO-d₆, 400 MHz) δ: 1.73 (s, 6H, 2N—C—CH₃); 2.40-2.44 (m, 2H, CH₂); 2.54-2.601 (m, 1.5H, one diastereoisomer of CH₂); 2.72-2.80 (m, 1H, one diastereoisomer of CH₂); 2.81-2.90 (m, 1.5H, one diastereoisomer of CH₂); 3.24 (s, 1.8H, one diastereoisomer of O—CH₃); 3.26 (s, 1.2H, one diastereoisomer of O—CH₃); 3.46-3.54 (m, 2H, CH₂); 3.83-3.92 (m, 0.8H, one diastereoisomer of CH); 4.20-4.26 (m 0.5H, one diastereoisomer of CH); 4.90-5.00 (m, 0.8H, one diastereoisomer of CH); 5.40-5.50 (m, 0.5H, one diastereoisomer of CH); 7.43 (d, J 8.2 Hz, 0.6H, one diastereoisomer of Ar); 7.44 (d, J 8.2 Hz, 0.4H, one diastereoisomer of Ar); 7.60-7.72 (m, 2H, Ar); 7.80-7.90 (m, 1H, Ar); 8.08-8.12 (m, 1H, NH); 8.46 (d, J 8.2 Hz, 1H, Ar); 8.50-8.53 (m, 1H, Ar).

M/Z (M[³⁵Cl]+H)⁺: 485.2

Mp: 92-98° C.

### Example 29: 4-(3-(4-chloro-3-fluorophenyl)-1-isobutyl-1H-pyrrolo[2,3-b]pyridine-6-carbonyl)-3,3-dimethylpiperazin-2-one

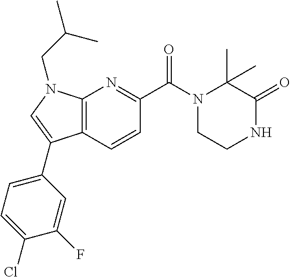 [SMILES: CC(C)Cn1cc(-c2ccc(Cl)c(F)c2)c2ccc(C(=O)N3CCNC(=O)C3(C)C)nc21]

Example 29 was prepared according to general procedure (Xa) starting from Compound 81 (65 mg) and 3,3-dimethylpiperazin-2-one (48 mg, 2 equiv), and using N,N-diisopropylethylamine (3.0 equiv). The crude was purified by flash chromatography (CyHex/EtOAC 70:30 to EtOAc 100%). The residue was taken up in a water ACN mixture and freeze-dried to obtain Example 29 (68 mg, 79%) as a white solid.

¹H-NMR (DMSO-d₆, 400 MHz) δ: 0.88 (d, J 6.7 Hz, 6H, 2 CH—CH₃); 1.72 (s, 6H, 2 CH₃); 2.17-2.28 (m, 1H, N—CH₂—CH); 3.30-3.38 (m, 2H, N—CH₂—CH₂—N); 3.52-3.58 (m, 2H, N—CH₂—CH₂—N); 4.12 (d, J 7.2 Hz, 2H, N—CH₂—CH); 7.46 (d, J 8.2 Hz, 1H, Ar); 7.60-7.67 (m, 2H, Ar); 7.74-7.80 (m, 1H, Ar); 8.07-8.12 (m, 1H, NH); 8.28 (s, 1H, Ar); 8.46 (d, J 8.2 Hz, 1H, Ar).

M/Z (M[³⁵Cl]+H)⁺: 457.2

Mp: 170-185° C.

### Example 30: 4-(3-(4-chloro-3-fluorophenyl)-1-(tetrahydro-2H-pyran-4-yl)-1H-pyrrolo[2,3-b]pyridine-6-carbonyl)-3,3-dimethylpiperazin-2-one

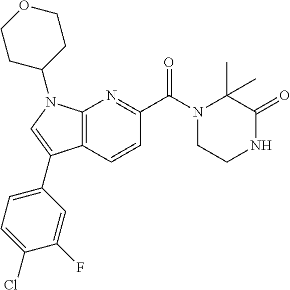 [SMILES: CC1(C)C(=O)NCCN1C(=O)c1ccc2c(-c3ccc(Cl)c(F)c3)cn(C3CCOCC3)c2n1]

Example 30 was prepared according to general procedure (Xa) starting from Compound 82 (75 mg) and 3,3-dimethylpiperazin-2-one (31 mg, 1.2 equiv), and using N,N-diisopropylethylamine (3.0 equiv). The crude was triturated in ACN (10 mL) then in Et₂O (10 mL) to obtain Example 30 (70 mg, 72%) as a beige solid.

¹H-NMR (DMSO-d₆, 400 MHz) δ: 1.73 (s, 6H, 2 CH₃); 1.91-1.98 (m, 2H, O—CH₂—CH₂); 2.10-2.22 (m, 2H, O—CH₂—CH₂); 3.30-3.36 (m, 2H, N—CH₂—CH₂—N); 3.52-3.62 (m, 4H, N—CH₂—CH₂—N+O—CH₂); 4.00-4.08 (m, 2H, O—CH₂); 4.91-5.01 (m, 1H, N—CH); 7.47 (d, J 8.2 Hz, 1H, Ar); 7.60-7.68 (m, 2H, Ar); 7.78-7.83 (m, 1H, Ar); 8.06-8.11 (m, 1H, NH); 8.43 (s, 1H, Ar); 8.46 (d, J 8.2 Hz, 1H, Ar).

M/Z (M[³⁵Cl]+H)⁺: 485.3

Mp: 220-224° C.

### Example 31: 4-(3-(4-chloro-3-fluorophenyl)-1-(2,2,2-trifluoroethyl)-1H-pyrrolo[2,3-b]pyridine-6-carbonyl)-3,3-dimethylpiperazin-2-one

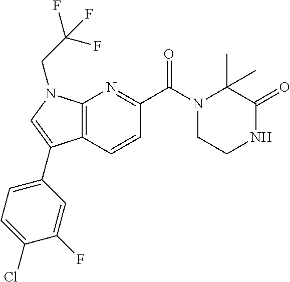 [SMILES: CC1(C)C(=O)NCCN1C(=O)c1ccc2c(-c3ccc(Cl)c(F)c3)cn(CC(F)(F)F)c2n1]

Example 31 was prepared according to general procedure (Xa) starting from Compound 83 (68 mg) and 3,3-dimethylpiperazin-2-one (47 mg, 2.0 equiv), and using N,N-diisopropylethylamine (3.0 equiv). The crude was purified by preparative HPLC (30% to 70% ACN/H₂O) to obtain Example 31 (65 mg, 74%) as a white solid.

¹H-NMR (DMSO-d₆, 400 MHz) δ: 1.72 (s, 6H, 2 CH₃); 3.30-3.34 (m, 2H, N—CH₂—CH₂—N); 3.47-3.53 (m, 2H, N—CH₂—CH₂—N); 5.23 (q, J 9.2 Hz, 2H, N—CH₂—CF₃); 7.54 (d, J 8.2 Hz, 1H, Ar); 7.62 (dd, J 8.5, 2.0 Hz, 1H, Ar); 7.65-7.70 (m, 1H, Ar); 7.79 (dd, J 11.0, 2.0 Hz, 1H, Ar); 8.07-8.11 (m, 1H, NH); 8.27 (s, 1H, Ar); 8.52 (d, J 8.2 Hz, 1H, Ar).

M/Z (M[³⁵Cl]+H)⁺: 483.1

Mp: 146-152° C.

### Example 32: (3-(4-chloro-3-fluorophenyl)-1-(2,2,2-trifluoroethyl)-1H-pyrrolo[2,3-b]pyridin-6-yl)(1,4-oxazepan-4-yl)methanone

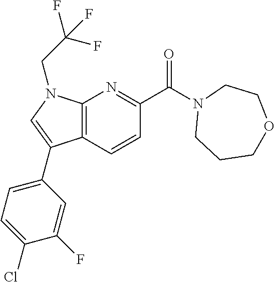 [SMILES: O=C(c1ccc2c(-c3ccc(Cl)c(F)c3)cn(CC(F)(F)F)c2n1)N1CCCOCC1]

Example 32 was prepared according to general procedure (Xa) starting from Compound 83 (60 mg) and homomorpholine (33 mg, 2 equiv), and using N,N-diisopropylethylamine (3.0 equiv). The crude was purified by flash chromatography (Interchim® 50 μm, CyHex 100% to CyHex/EtOAc 50:50). The residue was taken up in water ACN mixture and freeze-dried to obtain Example 32 (50 mg, 68%) as a white solid.

¹H-NMR (DMSO-d₆, 400 MHz) δ: 1.80-1.86 (m, 1H, CHₐH_{b}); 1.91-1.98 (m, 1H, CHₐH_{b}); 3.59-3.65 (m, 2H, CH₂); 3.70- 3.81 (m, 6H, 3 CH₂); 5.24 (q, J 9.2 Hz, 2H, N—CH₂—CF₃); 7.53 (d, J 8.2 Hz, 1H, Ar); 7.60-7.70 (m, 2H, Ar); 7.79 (m, 1H, Ar); 8.25 (s, 1H, Ar); 8.52 (d, J 8.2 Hz, 1H, Ar).

M/Z (M[³⁵Cl]+H)⁺: 456.1

Mp: 90-95° C.

### Example 33: (8-oxa-3-azabicyclo[3.2.1]octan-3-yl)(3-(4-chloro-3-fluorophenyl)-1-(2,2,2-trifluoroethyl)-1H-pyrrolo[2,3-b]pyridin-6-yl)methanone

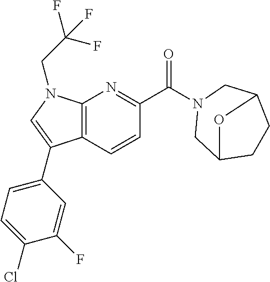 [SMILES: O=C(c1ccc2c(-c3ccc(Cl)c(F)c3)cn(CC(F)(F)F)c2n1)N1CC2CCC(C1)O2]

Example 33 was prepared according to general procedure (Xa) starting from Compound 83 (60 mg) and 8-oxa-3-azabicyclo[3.2.1]octane hydrochloride (48 mg, 2 equiv), and using N,N-diisopropylethylamine (4.0 equiv). The crude was purified by flash chromatography (Interchim® 50 μm, CyHex 100% to CyHex/EtOAc 35:65). The residue was taken up in water ACN mixture and freeze-dried to obtain Example 33 (50 mg, 66%) as a white solid.

¹H-NMR (DMSO-d₆, 400 MHz) δ: 1.72-2.10 (m, 4H, 2 CH₂); 3.02-3.10 (m, 1H, CHₐH_{b}); 3.30-3.36 (m, 1H, CHₐH_{b}); 3.58-3.66 (m, 1H, CHₐH_{b}); 4.14-4.23 (m, 2H, CH₂); 4.40-4.43 (m, 1H; CHₐH_{b}); 5.18-5.28 (m, 2H; N—CH₂—CF₃); 7.51 (d, J 8.2 Hz, 1H, Ar); 7.60-7.70 (m, 2H, Ar); 7.78 (m, 1H, Ar); 8.26 (s, 1H, Ar); 8.52 (d, J 8.2 Hz, 1H, Ar).

M/Z (M[³⁵Cl]+H)⁺: 468.1

Mp: 158-160° C.

### Example 34: 4-(3-(4-chloro-3-fluorophenyl)-1-methyl-1H-pyrrolo[2,3-b]pyridine-6-carbonyl)-3,3-dimethylpiperazin-2-one

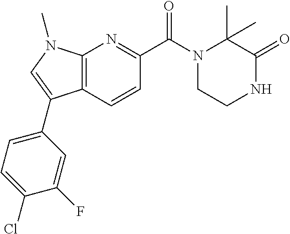 [SMILES: Cn1cc(-c2ccc(Cl)c(F)c2)c2ccc(C(=O)N3CCNC(=O)C3(C)C)nc21]

Example 34 was prepared according to general procedure (Xa) starting from Compound 84 (60 mg) and 3,3-dimethylpiperazin-2-one (50 mg, 2 equiv), and using N,N-diisopropylethylamine (3.0 equiv). The crude was purified by flash chromatography (Interchim® 50 μm, DCM 100% to DCM/MeOH 95:5). The residue was taken up in water ACN mixture and freeze-dried to obtain Example 34 (60 mg, 73%) as a white solid.

¹H-NMR (DMSO-d₆, 400 MHz) δ: 1.72 (s, 6H, 2 CH₃); 3.29-3.35 (m, 2H, N—CH₂—CH₂—N); 3.48-3.53 (m, 2H, N—CH₂—CH₂—N); 3.88 (s, 3H, N—CH₃); 7.43 (d, J 8.2 Hz, 1H, Ar); 7.59-7.66 (m, 2H, Ar); 7.72-7.76 (m, 1H, Ar); 8.07-8.12 (m, 1H, NH); 8.23 (s, 1H, Ar); 8.46 (d, J 8.2 Hz, 1H, Ar).

M/Z (M[³⁵Cl]+H)⁺: 415.2

Mp>250° C.

### Example 35: 4-(3-(4-chloro-3-fluorophenyl)-1-(cyclopropylmethyl)-1H-pyrrolo[2,3-b]pyridine-6-carbonyl)-3,3-dimethylpiperazin-2-one

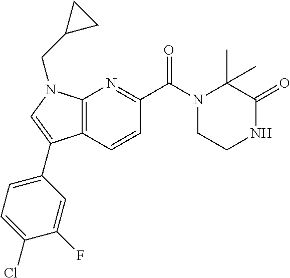 [SMILES: CC1(C)C(=O)NCCN1C(=O)c1ccc2c(-c3ccc(Cl)c(F)c3)cn(CC3CC3)c2n1]

Example 35 was prepared according to general procedure (Xa) starting from Compound 85 (65 mg) and 3,3-dimethylpiperazin-2-one (48 mg, 2 equiv), and using N,N-diisopropylethylamine (3.0 equiv). The crude was purified by flash chromatography (Interchim® 50 μm, CyHex/EtOAc 50:50 to CyHex/EtOAc 10:90) The residue was taken up in water ACN mixture and freeze-dried to obtain Example 35 (70 mg, 82%) as a white solid.

¹H-NMR (DMSO-d₆, 400 MHz) δ: 0.43-0.48 (m, 2H, N—CH₂—CH—CH₂); 0.50-0.57 (m, 2H, N—CH₂—CH—CH₂); 1.26-1.38 (m, 1H, N—CH₂—CH—CH₂); 1.73 (s, 6H, 2 CH₃); 3.28-3.35 (m, 2H, N—CH₂—CH₂—N); 3.48-3.54 (m, 2H, N—CH₂—CH₂—N); 4.17 (d, J 7.1 Hz, 2H, N—CH₂—CH—CH₂); 7.44 (d, J 8.2 Hz, 1H, Ar); 7.60-7.67 (m, 2H, Ar); 7.74-7.79 (m, 1H, Ar); 8.07-8.11 (m, 1H, NH); 8.34 (s, 1H, Ar); 8.46 (d, J 8.2 Hz, 1H, Ar).

M/Z (M[³⁵Cl]+H)⁺: 455.2

Mp: 153-159° C.

### Example 36: 4-(1-isobutyl-3-(pyrimidin-5-yl)-1H-pyrrolo[2,3-b]pyridine-6-carbonyl)-3,3-dimethylpiperazin-2-one

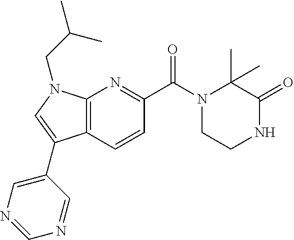 [SMILES: CC(C)Cn1cc(-c2cncnc2)c2ccc(C(=O)N3CCNC(=O)C3(C)C)nc21]

Example 36 was prepared according to general procedure (Xa) starting from Compound 86 (57 mg) and 3,3-dimethylpiperazin-2-one (49 mg, 2 equiv), and using N,N-diisopropylethylamine (3.0 equiv). The crude was purified by flash chromatography (Interchim® 50 μm, DCM 100% to DCM/MeOH 95:5) to obtain, after trituration in ACN (3 mL), Example 36 (49 mg, 63%) as a beige solid.

¹H-NMR (DMSO-d₆, 400 MHz) δ: 0.89 (d, J 6.7 Hz, 6H, 2N—CH₂—CH—CH₃); 1.73 (s, 6H, 2N—C—CH₃); 2.18-2.30 (m, 1H, N—CH₂—CH—CH₃); 3.30-3.36 (m, 2H, N—CH₂—CH₂—N); 3.52-3.58 (m, 2H, N—CH₂—CH₂—N); 4.15 (d, J 7.3 Hz, 2H, N—CH₂—CH—CH₃); 7.46 (d, J 8.2 Hz, 1H, Ar); 8.07-8.12 (m, 1H, NH); 8.40 (s, 1H, Ar); 8.54 (d, J 8.2 Hz, 1H, Ar); 9.09 (s, 1H, Ar); 9.20 (s, 2H, Ar).

M/Z (M+H)⁺: 407.2

Mp: 185-190° C.

### Example 37: 4-(1-isobutyl-3-(pyridin-4-yl)-1H-pyrrolo[2,3-b]pyridine-6-carbonyl)-3,3-dimethylpiperazin-2-one

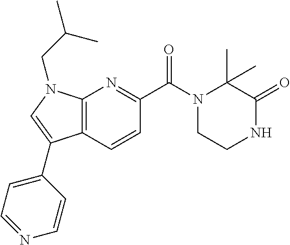 [SMILES: CC(C)Cn1cc(-c2ccncc2)c2ccc(C(=O)N3CCNC(=O)C3(C)C)nc21]

Example 37 was prepared according to general procedure (Xa) starting from Compound 87 (25 mg) and 3,3-dimethylpiperazin-2-one (22 mg, 2 equiv), and using N,N-diisopropylethylamine (3.0 equiv). The crude was purified by flash chromatography (Interchim® 50 μm, DCM 100% to DCM/MeOH 92:8), to obtain, after trituration in ACN (3 mL), Example 37 (14 mg, 41%) as a beige solid.

¹H-NMR (DMSO-d₆, 400 MHz) δ: 0.88 (d, J 6.7 Hz, 6H, 2N—CH₂—CH—CH₃); 1.72 (s, 6H, 2N—C—CH₃); 2.18-2.30 (m, 1H, N—CH₂—CH—CH₃); 3.30-3.36 (m, 2H, N—CH₂—CH₂—N); 3.52-3.58 (m, 2H, N—CH₂—CH₂—N); 4.14 (d, J 7.3 Hz, 2H, N—CH₂—CH—CH₃); 7.49 (d, J 8.2 Hz, 1H, Ar); 7.74-7.78 (m, 2H, Ar); 8.07-8.12 (m, 1H, NH); 8.45 (s, 1H, Ar); 8.54-8.60 (m, 3H, Ar).

M/Z (M+H)⁺: 406.3

Mp: 200-210° C.

### Example 38: 4-(3-(4-chloro-3-fluorophenyl)-1-isobutyl-2-methyl-1H-pyrrolo[2,3-b]pyridine-6-carbonyl)-3,3-dimethylpiperazin-2-one

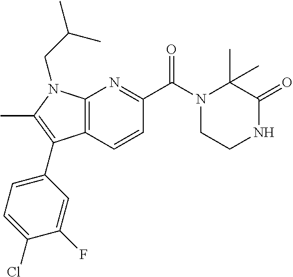 [SMILES: Cc1c(-c2ccc(Cl)c(F)c2)c2ccc(C(=O)N3CCNC(=O)C3(C)C)nc2n1CC(C)C]

Example 38 was prepared according to general procedure (Xa) starting from Compound 88 (40 mg) and 3,3-dimethylpiperazin-2-one (28 mg, 2 equiv), and using N,N-diisopropylethylamine (3.0 equiv). The crude was purified by flash chromatography (Interchim® 50 μm, CyHex 100% to CyHex/EtOAc 80:20) to obtain, after trituration in ACN (3 mL), Example 38 (27 mg, 52%) as a beige solid.

¹H-NMR (DMSO-d₆, 400 MHz) δ: 0.89 (d, J 6.7 Hz, 6H, 2N—CH₂—CH—CH₃); 1.72 (s, 6H, 2N—C—CH₃); 2.14-2.26 (m, 1H, N—CH₂—CH—CH₃); 2.57 (s, 3H, CH₃); 3.32-3.38 (m, 2H, N—CH₂—CH₂—N); 3.55-3.61 (m, 2H, N—CH₂—CH₂—N); 4.13 (d, J 7.6 Hz, 2H, N—CH₂—CH—CH₃); 7.37 (dd, J 8.2, 1.7 Hz, 1H, Ar); 7.41 (d, J 8.1 Hz, 1H, Ar); 7.53 (dd, J 10.6, 1.7 Hz, 1H, Ar); 7.68 (t, J 8.2 Hz, 1H, Ar); 8.03 (d, J 8.1 Hz, 1H, Ar); 8.07-8.10 (m, 1H, NH).

M/Z (M[³⁵Cl]+H)⁺: 471.2

Mp: 225-229° C.

### Example 39: 4-(3-(4-chloro-3-fluorophenyl)-1-(2-(2,2,2-trifluoroethoxy)ethyl)-1H-pyrrolo[2,3-b]pyridine-6-carbonyl)-3,3-dimethylpiperazin-2-one

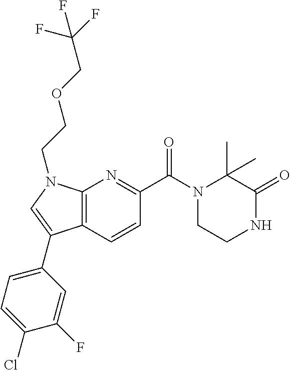 [SMILES: CC1(C)C(=O)NCCN1C(=O)c1ccc2c(-c3ccc(Cl)c(F)c3)cn(CCOCC(F)(F)F)c2n1]

Example 39 was prepared according to general procedure (Xa) starting from Compound 89 (85 mg) and 3,3-dimethylpiperazin-2-one (52 mg, 2 equiv), and using N,N-diisopropylethylamine (3.0 equiv). The crude was purified by flash chromatography (Interchim® 50 μm, DCM 100% to DCM/MeOH 95:5). The residue was taken up in water ACN mixture and freeze-dried to obtain Example 39 (70 mg, 65%) as a white solid.

¹H-NMR (DMSO-d₆, 400 MHz) δ: 1.72 (s, 6H, 2 CH₃); 3.30-3.34 (m, 2H, N—CH₂—CH₂—N); 3.49-3.55 (m, 2H, N—CH₂—CH₂—N); 4.03 (t, J 5.3 Hz, 2H, N—CH₂—CH₂—O); 4.10 (q, J 9.5 Hz, 2H, O—CH₂—CF₃); 4.52 (t, J 5.3 Hz, 2H, N—CH₂—CH₂-0); 7.46 (d, J 8.2 Hz, 1H, Ar); 7.60 (dd, J 8.3 1.9 Hz, 1H, Ar); 7.62-7.68 (m, 1H, Ar); 7.74 (dd, J 11.0 1.9 Hz, 1H, Ar); 8.08-8.13 (m, 1H, NH); 8.24 (s, 1H, Ar); 8.47 (d, J 8.2 Hz, 1H, Ar).

M/Z (M[³⁵Cl]+H)⁺: 527.2

Mp: 185-190° C.

### Example 40: 4-(1-(2-(1H-1,2,4-triazol-1-yl)ethyl)-3-(4-chloro-3-fluorophenyl)-1H-pyrrolo[2,3-b]pyridine-6-carbonyl)-3,3-dimethylpiperazin-2-one

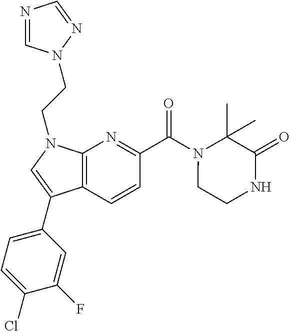 [SMILES: CC1(C)C(=O)NCCN1C(=O)c1ccc2c(-c3ccc(Cl)c(F)c3)cn(CCn3cncn3)c2n1]

Example 40 was prepared according to general procedure (Xa) starting from Compound 90 (35 mg) and 3,3-dimethylpiperazin-2-one (23 mg, 2 equiv), and using N,N-diisopropylethylamine (3.0 equiv). The crude was purified by flash chromatography (Interchim® 50 μm, DCM 100% to DCM/MeOH 92:8) to obtain Example 40 (20 mg, 44%) as a white solid.

¹H-NMR (DMSO-d₆, 400 MHz) δ: 1.73 (s, 6H, 2 CH₃); 3.33-3.38 (m, 2H, N—CH₂—CH₂—NH); 3.45-3.51 (m, 2H, N—CH₂—CH₂—NH); 4.69-4.78 (m, 4H, N—CH₂—CH₂—N); 7.44 (d, J 8.1 Hz, 1H, Ar); 7.55 (dd, J 8.2, 1.8 Hz, 1H, Ar); 7.64 (t, J 8.2 Hz, 1H, Ar); 7.69 (dd, J 11.0, 1.8 Hz, 1H, Ar); 7.87 (s, 1H, Ar); 8.00 (s, 1H, Ar); 8.10-8.15 (m, 1H, NH); 8.24 (s, 1H, Ar); 8.43 (d, J 8.1 Hz, 1H, Ar).

M/Z (M[³⁵Cl]+H)⁺: 496.2

Mp: 230-233° C.

### Example 41: 4-(3-(4-chloro-3-fluorophenyl)-1-(2-(2-oxopyrrolidin-1-yl)ethyl)-1H-pyrrolo[2,3-b]pyridine-6-carbonyl)-3,3-dimethylpiperazin-2-one

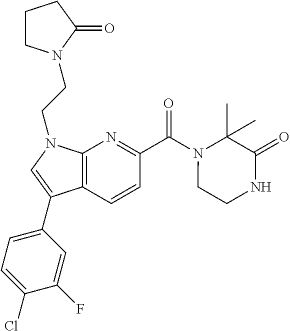 [SMILES: CC1(C)C(=O)NCCN1C(=O)c1ccc2c(-c3ccc(Cl)c(F)c3)cn(CCN3CCCC3=O)c2n1]

Example 41 was prepared according to general procedure (Xa) starting from Compound 91 (73 mg) and 3,3-dimethylpiperazin-2-one (46 mg, 2 equiv), and using N,N-diisopropylethylamine (3.0 equiv). The crude was purified by flash chromatography (Interchim® 50 μm, DCM 100% to DCM/MeOH 92:8) The residue was taken up in water ACN mixture and freeze-dried to obtain Example 41 (50 mg, 54%) as a white solid.

¹H-NMR (DMSO-d₆, 400 MHz) δ: 1.68-1.78 (m, 8H, 2 CH₃+N—CH₂—CH₂—CH₂—CO); 1.97 (t, J 7.9 Hz, 2H, N—CH₂—CH₂—CH₂—CO); 3.24 (t, J 6.9 Hz, 2H, N—CH₂—CH₂—CH₂—CO); 3.31-3.36 (m, 2H, N—CH₂—CH₂—N); 3.52-3.58 (m, 2H, N—CH₂—CH₂—N); 3.65 (t, J 5.7 Hz, 2H, N—CH₂—CH₂—N—CO); 4.44 (t, J 5.7 Hz, 2H, N—CH₂—CH₂—N—CO); 7.44 (d, J 8.1 Hz, 1H, Ar); 7.59-7.67 (m, 2H, Ar); 7.74 (dd, J 11.2 1.8 Hz, 1H, Ar); 8.08-8.13 (m, 1H, NH); 8.28 (s, 1H, Ar); 8.46 (d, J 8.1 Hz, 1H, Ar).

M/Z (M[³⁵Cl]+H)⁺: 512.2

Mp: 100-18° C.

### Example 42: 4-(3-(4-chloro-3-fluorophenyl)-1-(2-ethoxyethyl)-1H-pyrrolo[2,3-b]pyridine-6-carbonyl)-3,3-dimethylpiperazin-2-one

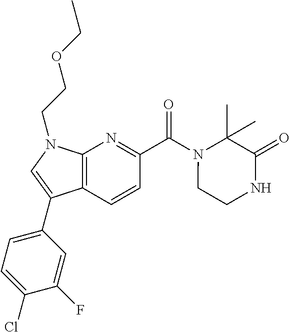 [SMILES: CCOCCn1cc(-c2ccc(Cl)c(F)c2)c2ccc(C(=O)N3CCNC(=O)C3(C)C)nc21]

Example 42 was prepared according to general procedure (Xa) starting from Compound 92 (61 mg) and 3,3-dimethylpiperazin-2-one (43 mg, 2 equiv), and using N,N-diisopropylethylamine (3.0 equiv). The crude was purified by flash chromatography (Interchim® 50 μm, DCM 100% to DCM/MeOH 96:4). The residue was taken up in water ACN mixture and freeze-dried to obtain Example 42 (40 mg, 50%) as a white solid.

¹H-NMR (DMSO-d₆, 400 MHz) δ: 1.03 (t, J 6.9 Hz, 3H, O—CH₂—CH₃); 1.72 (s, 6H, 2 CH₃); 3.32-3.35 (m, 2H, N—CH₂—CH₂—N); 3.44 (q, J 6.9 Hz, 2H, O—CH₂—CH₃); 3.49-3.54 (m, 2H, N—CH₂—CH₂—N); 3.79 (t, J 5.4 Hz, 2H, O—CH₂—CH₂—N); 4.46 (t, J 5.4 Hz, 2H, O—CH₂—CH₂—N); 7.45 (d, J 8.2 Hz, 1H, Ar); 7.58-7.67 (m, 2H, Ar); 7.73-7.77 (m, 1H, Ar); 8.06-8.11 (m, 1H, NH); 8.25 (s, 1H, Ar); 8.46 (d, J 8.2 Hz, 1H, Ar).

M/Z (M[³⁵Cl]+H)⁺: 473.2

Mp: 192-193° C.

### Compound 96: 3-bromo-1-isobutyl-1H-pyrrolo[2,3-b]pyridine-6-carboxamide

Compound 96 was obtained (654 mg, 97%) as a white solid according to general procedure (VII) starting from Compound 24 (635 mg).

M/Z (M[⁷⁹Br]+H)⁺: 296.0

### Compound 97: methyl 3-bromo-1-isobutyl-1H-pyrrolo[2,3-b]pyridine-6-carboxylate

Compound 97 was prepared according to general procedure (VIII) starting from Compound 96 (650 mg). The crude was purified by flash chromatography (CyHex 100% to CyHex/EtOAc 80:20) to obtain Compound 97 (680 mg, 100%) as a yellow oil.

M/Z (M[⁷⁹Br]+H)⁺: 311.0

### Compound 98: 3-bromo-1-isobutyl-1H-pyrrolo[2,3-b]pyridine-6-carboxylic acid

Compound 98 was obtained (645 mg, 100%) as a yellow oil according to general procedure (IXa) starting from Compound 97 (675 mg)

M/Z (M[⁷⁹Br]+H)⁺: 296.8

### Compound 99: 4-(3-bromo-1-isobutyl-1H-pyrrolo[2,3-b]pyridine-6-carbonyl)-3,3-dimethylpiperazin-2-one

Compound 99 was prepared according to general procedure (Xa) starting from Compound 98 (640 mg) and 3,3-dimethylpiperazin-2-one (552 mg, 2 equiv), and using N,N-diisopropylethylamine (3.0 equiv). The crude was purified by flash chromatography (CyHex 100% to CyHex/EtOAc 20:80). The resulting beige solid was triturated in 4 mL of ACN to obtain Compound 99 (683 mg, 78%) as a white solid.

M/Z (M[⁷⁹Br]+H)⁺: 407.1

### Example 43: 4-(1-isobutyl-3-(6-(trifluoromethyl)pyridin-3-yl)-1H-pyrrolo[2,3-b]pyridine-6-carbonyl)-3,3-dimethylpiperazin-2-one

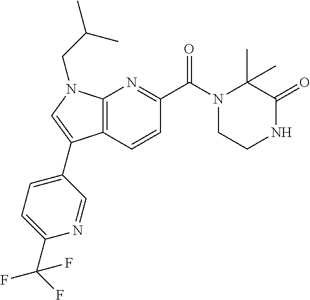 [SMILES: CC(C)Cn1cc(-c2ccc(C(F)(F)F)nc2)c2ccc(C(=O)N3CCNC(=O)C3(C)C)nc21]

Example 43 was prepared according to general procedure (XVIIa) starting from Compound 99 (90 mg) and 6-(trifluoromethyl)pyridin-3-yl boronic acid (51 mg, 1.2 equiv). The crude was purified by flash chromatography (Interchim® 50 μm, CyHex/EtOAc 50:50 to EtOAc 100%). The residue was taken up in water ACN mixture and freeze-dried to obtain Example 43 (62 mg, 59%) as a white solid.

¹H-NMR (DMSO-d₆, 400 MHz) δ: 0.89 (d, J 6.6 Hz, 6H, 2N—CH₂—CH—CH₃); 1.73 (s, 6H, 2N—C—CH₃); 2.20-2.30 (m, 1H, N—CH₂—CH—CH₃); 3.30-3.40 (m, 2H, N—CH₂—CH₂—N); 3.52-3.58 (m, 2H, N—CH₂—CH₂—N); 4.16 (d, J 7.3 Hz, 2H, N—CH₂—CH—CH₃); 7.50 (d, J 8.2 Hz, 1H, Ar); 7.94 (d, J 8.2 Hz, 1H, Ar); 8.10 (bs, 1H, NH); 8.40-8.45 (m, 1H, Ar); 8.47 (s, 1H, Ar); 8.55 (d, J 8.2 Hz, 1H, Ar); 9.16-9.20 (m, 1H, Ar).

M/Z (M+H)⁺: 474.2

Mp>250° C.

### Example 44: 4-(1-isobutyl-3-(1-methyl-1H-pyrazol-4-yl)-1H-pyrrolo[2,3-b]pyridine-6-carbonyl)-3,3-dimethylpiperazin-2-one

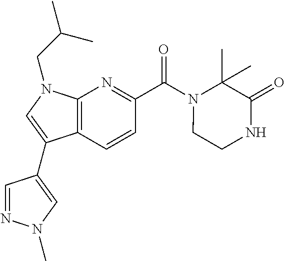 [SMILES: CC(C)Cn1cc(-c2cnn(C)c2)c2ccc(C(=O)N3CCNC(=O)C3(C)C)nc21]

Example 44 was prepared according to general procedure (XVIIa) starting from Compound 99 (90 mg) and 1-Methylpyrazole-4-boronic acid pinacol ester (55 mg, 1.2 equiv). The crude was purified by flash chromatography (Interchim® 50 μm, CyHex 100% to CyHex/EtOAc 50:50). The residue was taken up in water ACN mixture and freeze-dried to obtain Example 44 (26 mg, 29%) as a beige solid.

¹H-NMR (DMSO-d₆, 400 MHz) δ: 0.86 (d, J 6.7 Hz, 6H, 2 CH₂—CH—CH₃); 1.72 (s, 6H, 2N—C—CH₃); 2.12-2.22 (m, 1H, N—CH₂—CH—CH₃); 3.32-3.37 (m, 2H, N—CH₂—CH₂—N); 3.52-3.58 (m, 2H, N—CH₂—CH₂—N); 3.89 (s, 3H, N—CH₃); 4.07 (d, J 7.3 Hz, 2H, N—CH₂—CH—CH₃); 7.39 (d, J 8.2 Hz, 1H, Ar); 7.81-7.83 (m, 1H, Ar); 7.91 (s, 1H, 1H, Ar); 8.06-8.10 (m, 1H, NH); 8.16 (s, 1H, Ar); 8.31 (d, J 8.2 Hz, 1H, Ar).

M/Z (M+H)⁺: 409.3

Mp>250° C.

### Example 45: 4-(3-(3,6-dihydro-2H-pyran-4-yl)-1-isobutyl-1H-pyrrolo[2,3-b]pyridine-6-carbonyl)-3,3-dimethylpiperazin-2-one

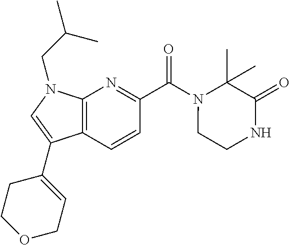 [SMILES: CC(C)Cn1cc(C2=CCOCC2)c2ccc(C(=O)N3CCNC(=O)C3(C)C)nc21]

Example 45 was prepared according to general procedure (XVIIa) starting from Compound 99 (125 mg) and 4-(4,4,5,5-Tetramethyl-1,3,2-dioxaborolan-2-yl)-3,6-dihydro-2Hpyran (77 mg, 1.2 equiv). The crude was purified by flash chromatography (Interchim® 50 μm, CyHex/EtOAc 50:50 to EtOAc 100%). The residue was taken up in water ACN mixture and freeze-dried to obtain Example 45 (76 mg, 60%) as a beige solid.

¹H-NMR (DMSO-d₆, 400 MHz) δ: 0.84 (d, J 7.0 Hz, 6H, 2 CH₂—CH—CH₃); 1.71 (s, 6H, 2N—C—CH₃); 2.12-2.23 (m, 1H, N—CH₂—CH); 2.50-2.53 (m, 2H, O—CH₂—CH₂); 3.32-3.37 (m, 2H, N—CH₂—CH₂—N); 3.52-3.58 (m, 2H, N—CH₂—CH₂—N); 3.85 (t, J 5.4 Hz, 2H, O—CH₂); 4.05 (d, J 7.0 Hz, 2H, N—CH₂—CH); 4.24-4.28 (m, 2H, O—CH₂); 6.26 (m, 1H, O—CH₂—CH); 7.38 (d, J 8.1 Hz, 1H, Ar); 7.78 (s, 1H, Ar); 8.05-8.09 (m, 1H, NH); 8.35 (d, J 8.1 Hz, 1H, Ar).

M/Z (M+H)⁺: 411.3

Mp: 225-230° C.

### Example 46: 4-(1-isobutyl-3-(tetrahydro-2H-pyran-4-yl)-1H-pyrrolo[2,3-b]pyridine-6-carbonyl)-3,3-dimethylpiperazin-2-one

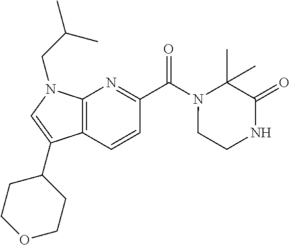 [SMILES: CC(C)Cn1cc(C2CCOCC2)c2ccc(C(=O)N3CCNC(=O)C3(C)C)nc21]

Example 46 was prepared by hydrogenation of Compound 100 (55 mg) in ethanol (1.3 mL) over Palladium on charcoal 10% wt (14 mg, 0.1 equiv) under one atmosphere of H₂ at 25° C. for 70 hours. The reaction mixture was filtered over a pad of Celite and the filtrate was concentrated. The residue was triturated in ACN (2 mL) then freeze-dried to obtain Example 46 (41 mg, 74%) as a white solid.

¹H-NMR (DMSO-d₆, 400 MHz) δ: 0.83 (d, J 7.0 Hz, 6H, 2 CH₂—CH—CH₃); 1.62-1.76 (m, 8H, 2N—C—CH₃+2 CH—CH₂—CH₂—O); 1.84-1.92 (m, 2H, CH—CH₂—CH₂—O); 2.10-2.20 (m, 1H, N—CH₂—CH—CH₃); 3.01-3.10 (m, 1H, O—CH₂—CH₂—CH); 3.28-3.4 (m, 2H, N—CH₂—CH₂—N); 3.45-3.55 (m, 4H, N—CH₂—CH₂—N+O—CH₂); 3.91-3.97 (m, 2H, O—CH₂); 4.01 (d, J 7.0 Hz, 2H, CH₂—CH—CH₃); 7.30 (d, J 8.1 Hz, 1H, Ar); 7.47 (s, 1H, Ar); 8.04-8.08 (m, 1H, NH); 8.12 (d, J 8.1 Hz, 1H, Ar).

M/Z (M+H)⁺: 413.3

Mp: >250° C.

### Compound 100: tert-butyl 4-(6-(2,2-dimethyl-3-oxopiperazine-1-carbonyl)-1-isobutyl-1H-pyrrolo[2,3-b]pyridin-3-yl)-3,6-dihydropyridine-1(2H)-carboxylate

Compound 100 was prepared according to general procedure (XVIIa) starting from Compound 99 (150 mg) and tert-butyl 4-(4,4,5,5-tetramethyl-1,3,2-dioxaborolan-2-yl)-3,6-dihydropyridine-1(2H)-carboxylate (137 mg, 1.2 equiv). The crude residue was purified by flash chromatography (Interchim® 50 μm, CyHex/EtOAc 50:50 to EtOAc 100%) to obtain Compound 100 (98 mg, 52%) as a yellow solid.

M/Z (M+H)⁺: 510.4

### Example 47: 4-(3-(1,2,3,6-tetrahydropyridin-4-yl)-1-isobutyl-1H-pyrrolo[2,3-b]pyridine-6-carbonyl)-3,3-dimethylpiperazin-2-one

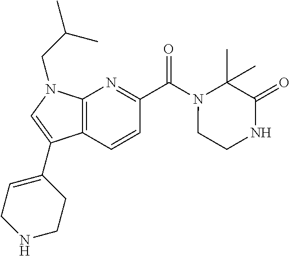 [SMILES: CC(C)Cn1cc(C2=CCNCC2)c2ccc(C(=O)N3CCNC(=O)C3(C)C)nc21]

Example 47 was obtained (30 mg, 39%) as a beige solid, according to general procedure (XVIIIb) starting from Compound 100 (95 mg).

¹H-NMR (DMSO-d₆, 400 MHz) δ: 0.84 (d, J 6.7 Hz, 6H, 2 CH₂—CH—CH₃); 1.71 (s, 6H, 2N—C—CH₃); 2.10-2.22 (m, 1H, N—CH₂—CH—CH₃); 2.48-2.54 (m, 2H, N—CH₂—CH₂—C); 3.04-3.10 (m, 2H, N—CH₂); 3.32-3.40 (m, 2H, N—CH₂—CH₂—N); 3.48-3.58 (m, 4H, N—CH₂+N—CH₂—CH₂—N); 4.05 (d, J 7.3 Hz, 2H, N—CH₂—CH—C); 6.24 (m, 1H, N—CH₂—CH); 7.38 (d, J 8.1 Hz, 1H, Ar); 7.77 (s, 1H, Ar); 8.05-8.10 (m, 1H, NH); 8.34 (d, J 8.1 Hz, 1H, Ar); NH signal not visible.

M/Z (M+H)⁺: 410.3

Mp: 140-145° C.

### Compound 101: 4-(3-(cyclohex-1-en-1-yl)-1-isobutyl-1H-pyrrolo[2,3-b]pyridine-6-carbonyl)-3,3-dimethylpiperazin-2-one

Compound 101 was prepared according to general procedure (XVIIa) starting from Compound 99 (125 mg) and 2-(cyclohex-1-en-1-yl)-4,4,5,5-tetramethyl-1,3,2-dioxaborolane (76 mg, 1.2 equiv). The crude residue was purified by flash chromatography (Interchim® 50 μm, CyHex/EtOAc 50:50 to EtOAc 100%) to obtain Compound 101 (80 mg, 64%) as a yellow solid.

M/Z (M+H)⁺: 409.3

### Example 48: 4-(3-cyclohexyl-1-isobutyl-1H-pyrrolo[2,3-b]pyridine-6-carbonyl)-3,3-dimethylpiperazin-2-one

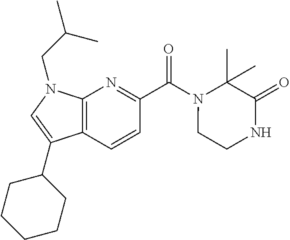 [SMILES: CC(C)Cn1cc(C2CCCCC2)c2ccc(C(=O)N3CCNC(=O)C3(C)C)nc21]

Example 48 was prepared by hydrogenation of Compound 101 (70 mg) in ethanol (1.7 mL) over Palladium on charcoal 10% wt (18 mg, 0.1 equiv) under one atmosphere of H₂ at 25° C. for 70 hours. The reaction mixture was filtered over a pad of Celite and the filtrate was concentrated. The crude was purified by flash chromatography (20 μm, DCM 100% to DCM/MeOH 85:15). The residual solid was triturated in DMSO (3 mL) then in water (3 mL) to obtain Example 48 (23 mg, 33%) as a white solid.

¹H-NMR (DMSO-d₆, 400 MHz) δ:

0.83 (d, J 6.7 Hz, 6H, 2 CH₂—CH—CH₃); 1.20-1.33 (m, 1H, CHₐH_{b}); 1.35-1.53 (m, 4H, 2 CH₂); 1.70 (s, 6H, 2 C—CH₃); 1.71-1.83 (m, 3H, CH₂+CHₐH_{b}); 1.95-2.02 (m, 2H, CH₂); 2.07-2.18 (m, 1H, N—CH₂—CH); 2.75-2.83 (m, 1H, CH₂—CH₂—CH); 3.31-3.35 (m, 2H, N—CH₂—CH₂—N); 3.48-3.55 (m, 2H, N—CH₂—CH₂—N); 4.00 (d, J 7.3 Hz, 2H, N—CH₂—CH); 7.29 (d, J 8.1 Hz, 1H, Ar); 7.40 (s, 1H, Ar); 8.04-8.08 (m, 1H, NH); 8.09 (d, J 8.1 Hz, 1H, Ar).

M/Z (M+H)⁺: 411.2

Mp: 226-229° C.

### Example 49: 4-(1-isobutyl-3-phenyl-pyrrolo[2,3-b]pyridine-6-carbonyl)-3,3-dimethyl-piperazin-2-one

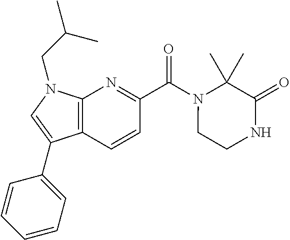 [SMILES: CC(C)Cn1cc(-c2ccccc2)c2ccc(C(=O)N3CCNC(=O)C3(C)C)nc21]

Example 49 was prepared according to general procedure (XVlIa) starting from Compound 99 (100 mg) and phenyl boronic acid (36 mg, 1.2 equiv). The crude residue was purified by flash chromatography (Merck 60®, CyHex 100% to CyHex/EtOAc 80:20) then by preparative HPLC (column B, 5% to 100% ACN/H₂O), to obtain Example 49 (40 mg, 41%) as a white solid.

¹H-NMR (DMSO-d₆, 300 MHz) δ: 0.88 (d, J 6.6 Hz, 6H, 2 CH₂—CH—CH₃), 1.72 (s, 6H, 2N—C—CH₃), 2.12-2.32 (m, 1H, N—CH₂—CH—CH₃), 3.32-3.39 (m, 2H, N—CH₂—CH₂—N), 3.50-3.61 (m, 2H, N—CH₂—CH₂—N), 4.11 (d, J 7.2 Hz, 2H, N—CH₂—CH—CH₃), 7.26 (t, J 7.3 Hz, 1H, Ar), 7.39-7.52 (m, 3H, Ar), 7.72 (d, J 7.3 Hz, 2H, Ar), 8.08-8.19 (m, 2H, Ar+NH), 8.41 (d, J 8.2 Hz, 1H, Ar).

M/Z (M+H)⁺: 405.2.

Mp: 175-178° C.

### Example 50: 4-[3-(3-chlorophenyl)-1-isobutyl-pyrrolo[2,3-b]pyridine-6-carbonyl]-3,3-dimethyl-piperazin-2-one

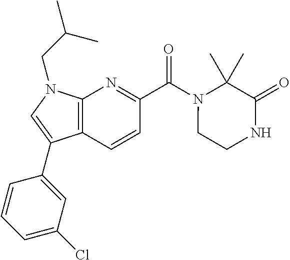 [SMILES: CC(C)Cn1cc(-c2cccc(Cl)c2)c2ccc(C(=O)N3CCNC(=O)C3(C)C)nc21]

Example 50 was prepared according to general procedure (XVIIa) starting from Compound 99 (100 mg) and 3-chlorophenyl boronic acid (46 mg, 1.2 equiv). The crude residue was purified by flash chromatography (Merck 60®, CyHex 100% to CyHex/EtOAc 70:30) then by preparative HPLC (column B, 5% to 100% ACN/H₂O), to obtain Example 50 (55 mg, 52%) as a white solid.

¹H-NMR (DMSO-d₆, 300 MHz) δ: 0.88 (d, J 6.6 Hz, 6H, 2 CH₂—CH—CH₃), 1.72 (s, 6H, 2N—C—CH₃), 2.13-2.28 (m, 1H, N—CH₂—CH—CH₃), 3.35-3.42 (m, 2H, N—CH₂—CH₂—N), 3.39-3.62 (m, 2H, N—CH₂—CH₂—N), 4.11 (d, J 7.2 Hz, 2H, N—CH₂—CH—CH₃), 7.32 (d, J 7.8 Hz, 1H, Ar), 7.41-7.54 (m, 2H, Ar), 7.73 (d, J 7.8 Hz, 1H, Ar), 7.76 (s, 1H, Ar), 8.12 (bs, 1H, NH), 8.27 (s, 1H, Ar), 8.43 (d, J 8.2 Hz, 1H, Ar).

M/Z (M[³⁵Cl]+H)⁺: 439.1.

Mp: 180-183° C.

### Example 51: 4-[1-isobutyl-3-(p-tolyl)pyrrolo[2,3-b]pyridine-6-carbonyl]-3,3-dimethyl-piperazin-2-one

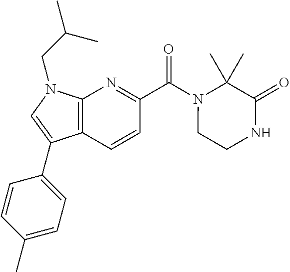 [SMILES: Cc1ccc(-c2cn(CC(C)C)c3nc(C(=O)N4CCNC(=O)C4(C)C)ccc23)cc1]

Example 51 was prepared according to general procedure (XVIIa) starting from Compound 99 (100 mg) and 4-tolyl boronic acid (40 mg, 1.2 equiv). The crude residue was purified by flash chromatography (Merck 60®, CyHex 100% to CyHex/EtOAc 70:30) then by preparative HPLC (column B, 5% to 100% ACN/H₂O), to obtain Example 51 (49 mg, 49%) as a white solid.

¹H-NMR (DMSO-d₆, 300 MHz) δ: 0.85 (d, J 6.6 Hz, 6H, 2 CH₂—CH—CH₃), 1.70 (s, 6H, 2N—C—CH₃), 2.11-2.25 (m, 1H, N—CH₂—CH—CH₃), 2.29 (s, 3H, CH₃), 3.48-3.59 (m, 2H, N—CH₂—CH₂—N), 4.11 (d, J 7.2 Hz, 2H, N—CH₂—CH—CH₃), 7.16-7.30 (m, 2H, Ar), 7.30-7.46 (m, 3H, Ar), 7.81 (s, 1H, Ar), 7.91 (d, J 8.1 Hz, 1H, Ar), 8.10 (bs, 1H, NH). N—CH₂—CH₂—N: 2 protons not observed.

M/Z (M+H)⁺: 419.2.

Mp: 207-212° C.

### Example 52: 4-[1-isobutyl-3-(o-tolyl)pyrrolo[2,3-b]pyridine-6-carbonyl]-3,3-dimethyl-piperazin-2-one

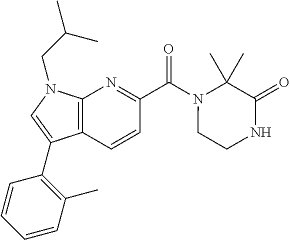 [SMILES: Cc1ccccc1-c1cn(CC(C)C)c2nc(C(=O)N3CCNC(=O)C3(C)C)ccc12]

Example 52 was prepared according to general procedure (XVIIa) starting from Compound 99 (100 mg) and 2-tolyl boronic acid (40 mg, 1.2 equiv). The crude residue was purified by flash chromatography (Merck 60®, CyHex 100% to CyHex/EtOAc 70:30) then by preparative HPLC (column B, 5% to 100% ACN/H₂O), to obtain Example 52 (46 mg, 46%) as a white solid.

¹H-NMR (DMSO-d₆, 300 MHz) δ: 0.85 (d, J 6.6 Hz, 6H, 2 CH₂—CH—CH₃), 1.69 (s, 6H, 2N—C—CH₃), 2.15-2.26 (m, 1H, N—CH₂—CH—CH₃), 2.32 (s, 3H, CH₃), 3.45-3.66 (m, 2H, N—CH₂—CH₂—N), 4.09 (d, J 7.2 Hz, 2H, N—CH₂—CH—CH₃), 7.24 (d, J 8.1 Hz, 2H, Ar), 7.40 (d, J 8.2 Hz, 1H, Ar), 7.59 (d, J 8.1 Hz, 2H, Ar), 8.04 (s, 1H, Ar), 8.09 (bs, 1H, NH), 8.35 (d, J 8.2 Hz, 1H, Ar). N—CH₂—CH₂—N: 2 protons not observed.

M/Z (M+H)⁺: 419.2.

Mp: 225-230° C.

### Example 53: 4-[3-(2-chlorophenyl)-1-isobutyl-pyrrolo[2,3-b]pyridine-6-carbonyl]-3,3-dimethyl-piperazin-2-one

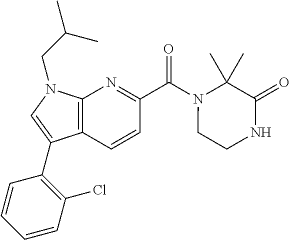 [SMILES: CC(C)Cn1cc(-c2ccccc2Cl)c2ccc(C(=O)N3CCNC(=O)C3(C)C)nc21]

Example 53 was prepared according to general procedure (XVIIa) starting from Compound 99 (100 mg) and 2-chlorophenyl boronic acid (46 mg, 1.2 equiv). The crude residue was purified by flash chromatography (Merck 60®, CyHex 100% to CyHex/EtOAc 90:10) then by preparative HPLC (column B, 5% to 100% ACN/H₂O), to obtain Example 53 (51 mg, 48%) as a white solid.

¹H-NMR (DMSO-d₆, 300 MHz) δ: 0.88 (d, J 6.6 Hz, 6H, 2 CH₂—CH—CH₃), 1.69 (s, 6H, 2N—C—CH₃), 2.12-2.30 (m, 1H, N—CH₂—CH—CH₃), 3.30-3.46 (m, 2H, N—CH₂—CH₂—N), 3.47-3.58 (m, 2H, N—CH₂—CH₂—N), 4.15 (d, J 7.2 Hz, 2H, N—CH₂—CH—CH₃), 7.29-7.52 (m, 3H, Ar), 7.59 (t, J 7.2 Hz, 2H, Ar), 7.91-8.08 (m, 2H, Ar+NH), 8.12 (bs, 1H, NH).

M/Z (M[³⁵Cl]+H)⁺: 439.2.

Mp: 189-192° C.

### Example 54: 4-[3-(4-chlorophenyl)-1-isobutyl-pyrrolo[2,3-b]pyridine-6-carbonyl]-3,3-dimethyl-piperazin-2-one

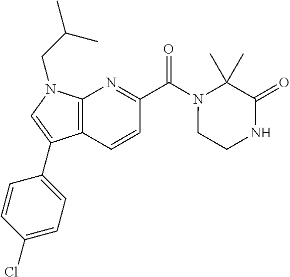 [SMILES: CC(C)Cn1cc(-c2ccc(Cl)cc2)c2ccc(C(=O)N3CCNC(=O)C3(C)C)nc21]

Example 54 was prepared according to general procedure (XVIIb) starting from Compound 99 (100 mg) and 4-chlorophenyl boronic acid (50 mg, 1.3 equiv). The crude residue was purified by flash chromatography (Merck 60®, CyHex 100% to EtOAc 100%) then triturated in ACN to obtain Example 54 (46 mg, 44%) as a white solid.

¹H-NMR (DMSO-d₆, 300 MHz) δ: 0.87 (d, J 6.6 Hz, 6H, 2 CH₂—CH—CH₃), 1.72 (s, 6H, 2N—C—CH₃), 2.15-2.31 (m, 1H, N—CH₂—CH—CH₃), 3.35-3.41 (m, 2H, N—CH₂—CH₂—N), 3.51-3.60 (m, 2H, N—CH₂—CH₂—N), 4.12 (d, J 7.3 Hz, 2H, N—CH₂—CH—CH₃), 7.45 (d, J 8.2 Hz, 1H, Ar), 7.47 (d, J 8.5 Hz, 2H, Ar), 7.76 (d, J 8.5 Hz, 2H, Ar), 8.12 (bs, 1H, NH), 8.19 (s, 1H, Ar), 8.41 (d, J 8.2 Hz, 1H, Ar).

M/Z (M[³⁵Cl]+H)⁺: 439.2.

Mp: 206-208° C.

### Example 55: 4-[1-isobutyl-3-[4-(trifluoromethyl)phenyl]pyrrolo[2,3-b]pyridine-6-carbonyl]-3,3-dimethyl-piperazin-2-one

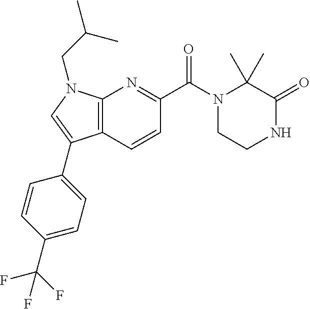 [SMILES: CC(C)Cn1cc(-c2ccc(C(F)(F)F)cc2)c2ccc(C(=O)N3CCNC(=O)C3(C)C)nc21]

Example 55 was prepared according to general procedure (XVIIb) starting from Compound 99 (100 mg) and 4-trifluoromethylphenyl boronic acid (90 mg, 2.0 equiv). The crude residue was purified by flash chromatography (Merck 60®, CyHex 100% to EtOAc 100%) then recrystallized from ACN to obtain Example 55 (49 mg, 43%) as a white solid.

¹H-NMR (DMSO-d₆, 300 MHz) δ: 0.88 (d, J 6.7 Hz, 6H, 2 CH₂—CH—CH₃), 1.72 (s, 6H, 2N—C—CH₃), 2.15-2.27 (m, 1H, N—CH₂—CH—CH₃), 3.33-3.39 (m, 2H, N—CH₂—CH₂—N), 3.50-3.60 (m, 2H, N—CH₂—CH₂—N), 4.14 (d, J 7.2 Hz, 2H, N—CH₂—CH—CH₃), 7.48 (d, J 8.1 Hz, 1H, Ar), 7.79 (d, J 8.4 Hz, 2H, Ar), 7.97 (d, J 8.1 Hz, 2H, Ar), 8.13 (bs, 1H, NH), 8.34 (s, 1H, Ar), 8.49 (d, J 8.2 Hz, 1H, Ar).

M/Z (M+H)⁺: 473.3.

Mp: 216-218° C.

### Example 56: 4-[1-isobutyl-3-[4-fluorophenyl]pyrrolo[2,3-b]pyridine-6-carbonyl]-3,3-dimethyl-piperazin-2-one

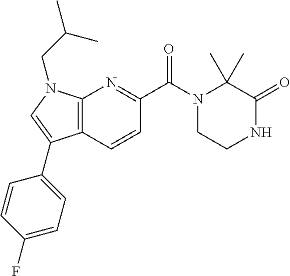 [SMILES: CC(C)Cn1cc(-c2ccc(F)cc2)c2ccc(C(=O)N3CCNC(=O)C3(C)C)nc21]

Example 56 was prepared according to general procedure (XVIIb) starting from Compound 99 (100 mg) and 4-fluorophenyl boronic acid (70 mg, 2.0 equiv). The crude residue was purified by flash chromatography (Merck 60®, CyHex 100% to EtOAc 100%) then recrystallized from ACN to obtain Example 56 (63 mg, 62%) as a white solid.

¹H-NMR (DMSO-d₆, 300 MHz) δ: 0.87 (d, J 6.6 Hz, 6H, 2 CH₂—CH—CH₃), 1.72 (s, 6H, 2N—C—CH₃), 2.12-2.38 (m, 1H, N—CH₂—CH—CH₃), 3.34-3.39 (m, 2H, N—CH₂—CH₂—N), 3.48-3.61 (m, 2H, N—CH₂—CH₂—N), 4.11 (d, J 7.3 Hz, 2H, N—CH₂—CH—CH₃), 7.29 (t, J 8.9 Hz, 2H, Ar), 7.44 (d, J 8.2 Hz, 2H, Ar), 7.70-7.81 (m, 2H, Ar), 8.10 (s, 1H, Ar), 8.11 (bs, 1H, NH), 8.38 (d, J 8.2 Hz, 1H, Ar).

M/Z (M+H)⁺: 423.3.

Mp: 192-194° C.

### Example 57: 4-[1-isobutyl-3-[3-(trifluoromethoxy)phenyl]pyrrolo[2,3-b]pyridine-6-carbonyl]-3,3-dimethyl-piperazin-2-one

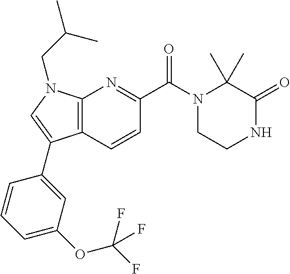 [SMILES: CC(C)Cn1cc(-c2cccc(OC(F)(F)F)c2)c2ccc(C(=O)N3CCNC(=O)C3(C)C)nc21]

Example 57 was prepared according to general procedure (XVIIb) starting from Compound 99 (100 mg) and 3-trifluoromethoxyphenyl boronic acid (100 mg, 2.0 equiv). The crude residue was purified by flash chromatography (Merck 60®, CyHex 100% to EtOAc 100%) then recrystallized from ACN to obtain Example 57 (49 mg, 42%) as a white solid.

¹H-NMR (CD₃OD, 300 MHz) δ: 0.93 (d, J 14.1 Hz, 6H, 2 CH₂—CH—CH₃), 1.87 (s, 6H, 2N—C—CH₃), 2.23-2.37 (m, 1H, N—CH₂—CH—CH₃), 3.44-3.52 (m, 2H, N—CH₂—CH₂—N), 3.65-3.72 (m, 2H, N—CH₂—CH₂—N), 4.19 (d, J 7.4 Hz, 2H, N—CH₂—CH—CH₃), 7.19 (dd, J 9.4, 1.1 Hz, 1H, Ar), 7.45-7.57 (m, 3H, Ar), 7.71 (d, J 8.2 Hz, 1H, Ar), 7.96 (s, 1H, Ar), 8.37 (d, J 8.2 Hz, 1H, Ar). N—H proton not observed.

M/Z (M+H)⁺: 489.3.

Mp: 202-204° C.

### Example 58: methyl 6-(4-(3-(tert-butyl)-1-(4-chloro-3-fluorophenyl)-1H-pyrazolo[4,3-b]pyridine-5-carbonyl)-3,3-dimethylpiperazin-1-yl)-2,4-dimethylnicotinate

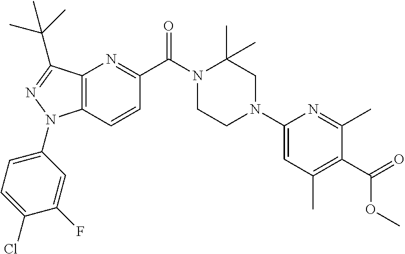 [SMILES: COC(=O)c1c(C)cc(N2CCN(C(=O)c3ccc4c(n3)c(C(C)(C)C)nn4-c3ccc(Cl)c(F)c3)C(C)(C)C2)nc1C]

Example 58 was prepared according to general procedure (Xa) starting from Compound 93 (130 mg) and Compound 95 (153 mg, 1.3 equiv), and using N,N-diisopropylethylamine (4.0 equiv). The crude was purified by flash chromatography (Interchim® 50 μm, CyHex 100% to CyHex/EtOAc 80:20) to obtain Example 58 (153 mg, 67%) as a yellow solid.

¹H-NMR (DMSO-d₆, 400 MHz) δ: 1.55 (s, 6H, 2N—C—CH₃); 1.60 (s, 9H, 3 CH₃); 2.25 (s, 3H, Pyr-CH₃); 2.36 (s, 3H, Pyr-CH₃); 3.55-3.62 (m, 2H, CH₂); 3.79 (s, 3H, O—CH₃); 3.83-3.88 (m, 2H, N—CH₂—CH₂—N); 3.93 (s, 2H, N—CH₂—CH₂—N); 6.35 (s, 1H, Ar); 7.71 (d, J 8.8 Hz, 1H, Ar); 7.72-7.75 (m, 1H, Ar); 7.81 (t, J 8.2 Hz, 1H, Ar); 7.91 (dd, J 10.5, 2.5 Hz, 1H, Ar); 8.48 (d, J 8.8 Hz, 1H, Ar).

M/Z (M[³⁵Cl]+H)⁺: 607.4

Mp: 152-158° C.

### Example 59: 6-(4-(3-(tert-butyl)-1-(4-chloro-3-fluorophenyl)-1H-pyrazolo[4,3-b]pyridine-5-carbonyl)-3,3-dimethylpiperazin-1-yl)-2,4-dimethylnicotinic acid

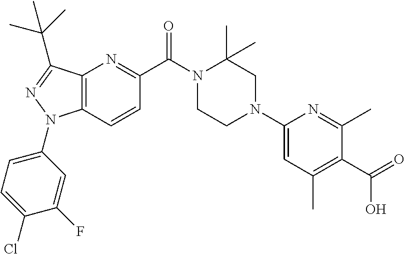 [SMILES: Cc1cc(N2CCN(C(=O)c3ccc4c(n3)c(C(C)(C)C)nn4-c3ccc(Cl)c(F)c3)C(C)(C)C2)nc(C)c1C(=O)O]

Example 59 was prepared according to general procedure (XI) starting from Example 58 (140 mg). The crude was purified by preparative HPLC (30% to 70% ACN/H₂O) to obtain Example 59 (40 mg, 29%) as a white solid.

¹H-NMR (DMSO-d₆, 400 MHz) δ: 1.55 (s, 6H, 2N—C—CH₃); 1.61 (s, 9H, 3 CH₃); 2.27 (s, 3H, Pyr-CH₃); 2.39 (s, 3H, Pyr-CH₃); 3.55-3.62 (m, 2H, CH₂); 3.83-3.88 (m, 2H, CH₂); 3.93 (s, 2H, CH₂); 6.32 (s, 1H, Ar); 7.71 (d, J 8.8 Hz, 1H, Ar); 7.72-7.75 (m, 1H, Ar); 7.81 (t, J 8.2 Hz, 1H, Ar); 7.91 (dd, J 10.5, 2.5 Hz, 1H, Ar); 8.48 (d, J 8.8 Hz, 1H, Ar); 12.64 (bs, 1H, COOH).

M/Z (M[³⁵Cl]+H)⁺: 593.3

Mp: 127-139° C.

### Example 60: 6-(4-(3-(tert-butyl)-1-(4-chloro-3-methoxyphenyl)-1H-pyrazolo[4,3-b]pyridine-5-carbonyl)-3,3-dimethylpiperazin-1-yl)-2,4-dimethylnicotinic acid

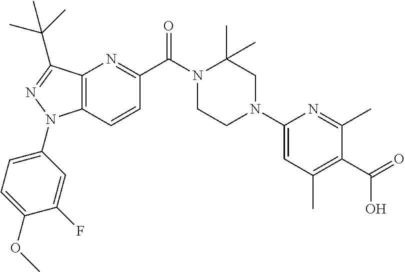 [SMILES: COc1ccc(-n2nc(C(C)(C)C)c3nc(C(=O)N4CCN(c5cc(C)c(C(=O)O)c(C)n5)CC4(C)C)ccc32)cc1F]

Example 60 was prepared according to general procedure (XI) starting from Example 58 (140 mg). The crude was purified by preparative HPLC (30% to 70% ACN/H₂O) to obtain Example 60 (30 mg, 21%) as a white solid.

¹H-NMR (DMSO-d₆, 400 MHz) δ: 1.55 (s, 6H, 2N—C—CH₃); 1.61 (s, 9H, 3 CH₃); 2.28 (s, 3H, Pyr-CH₃); 2.39 (s, 3H, Pyr-CH₃); 3.55-3.62 (m, 2H, CH₂); 3.83-3.88 (m, 2H, CH₂); 3.93 (s, 2H, CH₂); 3.99 (s, 3H, O—CH₃); 6.32 (s, 1H, Ar); 7.37 (dd, J 8.5, 2.4 Hz, 1H, Ar); 7.50 (d, J 2.4 Hz, 1H, Ar); 7.64 (d, J 8.5 Hz, 1H, Ar); 7.68 (d, J 8.8 Hz, 1H, Ar); 8.42 (d, J 8.8 Hz, 1H, Ar); 12.62 (bs, 1H, COOH).

M/Z (M+H)⁺: 605.3

Mp: 127-139° C.

### Compound 102: 2-(1H-pyrazol-1-yl)ethyl methanesulfonate

Compound 102 was obtained (370 mg, n.d.) as a yellow oil according to general procedure (XXIII) from 2-pyrazol-1-ylethanol (167 mg) and methanesulfonyl chloride (173 μL, 1.5 equiv).

¹H-NMR (DMSO-d₆, 400 MHz) δ: 3.04 (s, 3H, CH₃); 4.40-4.46 (m, 2H, O—CH₂); 4.50-4.56 (m, 2H, N—CH₂); 6.25-6.28 (m, 1H, Ar); 7.48-7.50 (m, 1H, Ar); 7.76-7.78 (m, 1H, Ar).

### Compound 103: 1-(2-(1H-pyrazol-1-yl)ethyl)-3-bromo-1H-pyrrolo[2,3-b]pyridine-6-carbonitrile

Compound 103 was prepared according to general procedure (XIII) in DMA from Compound 23 (165 mg) and Compound 102 (283 mg, 2 equiv). The reaction mixture was heated at 70° C. for 1 hour. The crude was purified by flash chromatography (Interchim® 50 μm, CyHex 100% to CyHex/EtOAc 60:40) to obtain Compound 103 (114 mg, 49%) as a colorless oil.

M/Z (M[⁷⁹Br]+H)⁺: 316.0

### Compound 104: 1-(2-(1H-pyrazol-1-yl)ethyl)-3-(4-chloro-3-fluorophenyl)-1H-pyrrolo[2,3-b]pyridine-6-carbonitrile

Compound 104 was prepared according to general procedure (XVIIa) starting from Compound 103 (114 mg) and (4-chloro-3-fluorophenyl) boronic acid (75 mg, 1.2 equiv). The crude residue was purified by flash chromatography (Interchim® 50 μm, CyHex 100% to CyHex/EtOAc 60:40) to obtain Compound 104 (73 mg, 55%) as a beige solid.

M/Z (M[³⁵Cl]+H)⁺: 366.1

### Compound 105: 1-(2-(1H-pyrazol-1-yl)ethyl)-3-(4-chloro-3-fluorophenyl)-1H-pyrrolo[2,3-b]pyridine-6-carboxamide

Compound 105 was obtained (71 mg, 97%) as a yellow solid according to general procedure (VII) starting from Compound 104 (70 mg).

M/Z (M[³⁵Cl]+H)⁺: 384.1

### Compound 106: methyl 1-(2-(1H-pyrazol-1-yl)ethyl)-3-(4-chloro-3-fluorophenyl)-1Hpyrrolo[2,3-b]pyridine-6-carboxylate

Compound 106 was prepared according to general procedure (VIII) starting from Compound 105 (70 mg). The crude was purified by flash chromatography (Interchim® 50 μm, DCM 100% to DCM/MeOH 95:5) to obtain Compound 106 (72 mg, 99%) as a yellow solid.

M/Z (M[³⁵Cl]+H)⁺: 399.1

### Compound 107: lithium 1-(2-(1H-pyrazol-1-yl)ethyl)-3-(4-chloro-3-fluorophenyl)-1H-pyrrolo[2,3-b]pyridine-6-carboxylate

Compound 107 was obtained (75 mg, n.d.) as a yellow solid according to general procedure (IXb) starting from Compound 106 (70 mg).

M/Z (M[³⁵Cl]+H)⁺: 385.0

### Example 61: 4-(1-(2-(1H-pyrazol-1-yl)ethyl)-3-(4-chloro-3-fluorophenyl)-1H-pyrrolo[2,3-b]pyridine-6-carbonyl)-3,3-dimethylpiperazin-2-one

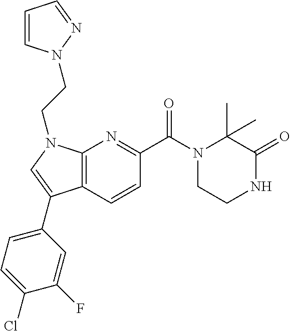 [SMILES: CC1(C)C(=O)NCCN1C(=O)c1ccc2c(-c3ccc(Cl)c(F)c3)cn(CCn3cccn3)c2n1]

Example 61 was prepared according to general procedure (Xa) starting from Compound 107 (70 mg) and 3,3-dimethylpiperazin-2-one (46 mg, 2 equiv), and using N,N-diisopropylethylamine (3.0 equiv). The crude was purified by flash chromatography (Interchim® 50 μm, DCM 100% to DCM/MeOH 92:8). The residue was triturated successively in ACN (2 mL) and Et₂O (2 mL) to obtain Example 61 (40 mg, 45%) as a white solid.

¹H-NMR (DMSO-d₆, 400 MHz) δ: 1.73 (s, 6H, 2 C—CH₃); 3.34-3.39 (m, 2H, N—CH₂—CH₂—N—CO); 3.48-3.53 (m, 2H, N—CH₂—CH₂—N—CO); 4.60-4.65 (m, 2H, N—CH₂); 4.70-4.76 (m, 2H, N—CH₂); 6.10 (t, J 2.0 Hz, 1H, Ar); 7.38-7.43 (m, 2H, Ar); 7.45 (d, J 8.2 Hz, 1H, Ar); 7.53 (dd, J 8.3, 1.8 Hz, 1H, Ar); 7.61-7.69 (m, 2H, Ar); 7.88 (s, 1H, Ar); 8.12 (bs, 1H, NH); 8.43 (d, J 8.2 Hz, 1H, Ar) M/Z (M[³⁵Cl]+H)⁺: 495.3

Mp: 210-216° C.

### Compound 108: 1-(3-fluoropyridin-2-yl)-3-methylbutan-1-one

Compound 108 was prepared according to general procedure (XXIVb) starting from 2-bromo-3-fluoropyridine (1.00 g) and N-methoxy-N,3-dimethylbutanamide (1.80 g, 2.0 equiv). The crude residue was purified by flash chromatography (Merck 60®, CyHex 100% to EtOAc 100%) to obtain Compound 108 (0.96 g, 93%) as a yellow oil.

M/Z (M+H)⁺: 182.2

### Compound 109: 2-(1-(2-(4-chloro-3-fluorophenyl)hydrazineylidene)-3-methylbutyl)-3-fluoropyridine

Compound 109 was prepared according to general procedure (XXVa) starting from Compound 108 (600 mg) and (4-chloro-3-fluorophenyl)hydrazine hydrochloride (798 mg, 1.5 equiv). The crude residue was purified by flash chromatography (Merck 60®, DCM 100% to DCM/(MeOH, NH₃ (7M)) 90:10) to obtain Compound 109 (1.4 g, n.d.) as a white solid.

M/Z (M[³⁵Cl]+H)⁺: 324.2

### Compound 110: 1-(4-chloro-3-fluorophenyl)-3-isobutyl-1H-pyrazolo[4,3-b]pyridine

Compound 110 was prepared according to general procedure (XXVII) starting from Compound 109 (350 mg). The crude residue was purified by flash chromatography (Merck 60®, CyHex 100% to CyHex/EtOAc 90:10) to obtain Compound 110 (200 mg, 60%) as a yellow oil.

M/Z (M[³⁵Cl]+H)⁺: 304.2

### Compound 111: 1-(4-chloro-3-fluorophenyl)-3-isobutyl-1H-pyrazolo[4,3-b]pyridine-4-oxide

Compound 111 was prepared according to general procedure (XIXb) starting from Compound 110 (600 mg). The crude residue was purified by flash chromatography (Merck 60®, DCM 100% to DCM/MEOH 90:10) to obtain Compound 111 (470 mg, 75%) as a yellow solid.

M/Z (M[³⁵Cl]+H)⁺: 320.2

### Compound 112: 1-(4-chloro-3-fluorophenyl)-3-isobutyl-1H-pyrazolo[4,3-b]pyridine-5-carbonitrile

Compound 112 was prepared according to general procedure (XX) starting from Compound 111 (600 mg). The crude residue was purified by flash chromatography (Merck 60®, CyHex 100% to CyHex/EtOAc 80:20) to obtain Compound 112 (370 mg, 71%) as a white solid.

M/Z (M[³⁵Cl]+H)⁺: 329.2

### Compound 113: 1-(4-chloro-3-fluorophenyl)-3-isobutyl-1H-pyrazolo[4,3-b]pyridine-5-carboxamide

Compound 113 was obtained (750 mg, n.d.) as a white solid according to general procedure (VII) starting from Compound 112 (530 mg).

M/Z (M[³⁵Cl]+H)⁺: 347.2

### Compound 114: methyl 1-(4-chloro-3-fluorophenyl)-3-isobutyl-1H-pyrazolo[4,3-b]pyridine-5-carboxylate

Compound 114 was prepared according to general procedure (VII) starting from Compound 113 (750 mg). The crude was purified by flash chromatography (Merck 60®, CyHex 100% to CyHex/EtOAc 80:20) to obtain Compound 114 (500 mg, 86%) as a white solid.

M/Z (M[³⁵Cl]+H)⁺: 362.2

### Compound 115: 1-(4-chloro-3-fluorophenyl)-3-isobutyl-1H-pyrazolo[4,3-b]pyridine-5-carboxylic acid

Compound 115 was obtained (480 mg, 99%) as a white solid according to general procedure (IXa) starting from Compound 114 (500 mg).

M/Z (M[³⁵Cl]+H)⁺: 348.2

### Example 62: 4-(1-(4-chloro-3-fluorophenyl)-3-isobutyl-1H-pyrazolo[4,3-b]pyridine-5-carbonyl)-3,3-dimethylpiperazin-2-one

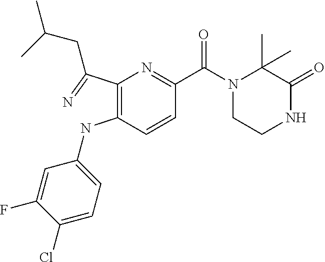 [SMILES: CC(C)CC(=N)c1nc(C(=O)N2CCNC(=O)C2(C)C)ccc1Nc1ccc(Cl)c(F)c1]

Example 62 was prepared according to general procedure (Xa) starting from Compound 115 (120 mg) and 3,3-dimethylpiperazin-2-one (89 mg, 2 equiv), and using N,N-diisopropylethylamine (3.0 equiv). The crude was purified by preparative HPLC (column B, 5% to 100% ACN/H₂O), to obtain Example 62 (110 mg, 71%) as a white solid.

¹H-NMR (DMSO-d₆, 300 MHz) δ: 0.98 (d, J 6.6 Hz, 6H, 2 CH—CH₃), 1.72 (s, 6H, 2 CH₃), 2.18-2.33 (m, 1H, CH—CH₃), 2.94 (d, J 7.0 Hz, 2H, CH₂—CH), 3.45-3.54 (m, 2H, N—CH₂), 7.69-7.84 (m, 3H, Ar), 7.91 (dd, J 10.5, 2.4 Hz, 1H, Ar), 8.13 (bs, 1H, NH), 8.50 (d, J 8.8 Hz, 1H, Ar), N—CH₂ signal not observed.

M/Z (M[³⁵Cl]+H)⁺: 458.3.

Mp: 215-218° C.

### Example 63: 4-(1-(4-chloro-3-fluorophenyl)-3-isobutyl-1H-pyrazolo[4,3-b]pyridine-5-carbonyl)-1-methylpiperazin-2-one

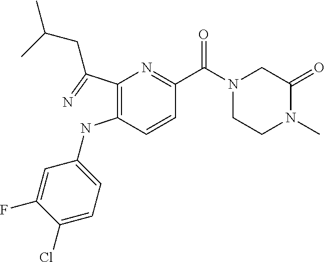 [SMILES: CC(C)CC(=N)c1nc(C(=O)N2CCN(C)C(=O)C2)ccc1Nc1ccc(Cl)c(F)c1]

Example 63 was prepared according to general procedure (Xa) starting from Compound 115 (60 mg) and 1-methylpiperazin-2-one (39 mg, 2 equiv), and using N,N-diisopropylethylamine (3.0 equiv). The crude was purified by preparative HPLC (column B, 5% to 100% ACN/H₂O), to obtain Example 63 (27 mg, 36%) as a white solid.

¹H-NMR (DMSO-d₆, 400 MHz) δ: 0.99 (d, J 6.6 Hz, 6H, 2 C—CH₃), 2.21-2.31 (m, 1H, CH—CH₃), 2.86-2.93 (m, 3H, N—CH₃), 2.96 (d, J 7.0 Hz, 2H, CH₂—CH), 3.42-3.50 (m, 2H, N—CH₂), 3.86-3.98 (m, 2H, N—CH₂), 4.23-4.32 (m, 2H, N—CH₂—CO), 7.71-7.75 (m, 1H, Ar), 7.78-7.85 (m, 2H, Ar), 7.92 (dd, J 10.4, 2.4 Hz, 1H, Ar), 8.52 (dd, J 8.8, 7.1 Hz, 1H, Ar).

M/Z (M[³⁵Cl]+H)⁺: 444.3.

Mp: 160-165° C.

### Example 64: methyl 6-(4-(1-(4-chloro-3-fluorophenyl)-3-isobutyl-1H-pyrazolo[4,3-b]pyridine-5-carbonyl)-3,3-dimethylpiperazin-1-yl)-2,4-dimethylnicotinate

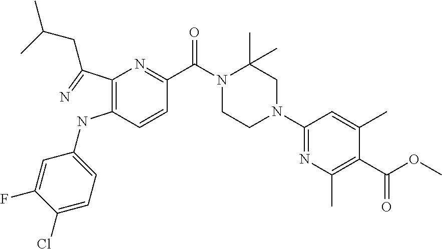 [SMILES: COC(=O)c1c(C)cc(N2CCN(C(=O)c3ccc(Nc4ccc(Cl)c(F)c4)c(C(=N)CC(C)C)n3)C(C)(C)C2)nc1C]

Example 64 was prepared according to general procedure (Xa) starting from Compound 115 (100 mg) and Compound 95 (160 mg, 2.0 equiv), and using N,N-diisopropylethylamine (3.0 equiv). The crude was purified by flash chromatography (Merck 60®, CyHex 100% to EtOAc 100%) to obtain Example 64 (156 mg, 90%) as a yellow oil.

¹H-NMR (DMSO-d₆, 400 MHz) δ: 1.05 (d, J 6.5 Hz, 6H, 2 CH—CH₃); 1.66 (s, 6H, 2 C—CH₃); 2.04 (s, 3H, Pyr-CH₃); 2.33 (s, 3H, Pyr-CH₃); 2.46-2.52 (m, 2H, N—CH₂—CH₂—N); 3.03 (d, J 7.1 Hz, 2H, CH—CH₂); 3.60-3.73 (m, 2H, N—CH₂—CH₂—N); 3.87 (s, 3H, O—CH₃); 3.97-4.05 (m, 2H, N—CH₂); 6.12 (bs, 1H, Ar); 7.45-7.51 (m, 1H, Ar); 7.52-7.61 (m, 2H, Ar); 7.82 (d, J 8.8 Hz, 1H, Ar); 8.09 (d, J 8.8 Hz, 1H, Ar); M/Z (M[³⁵Cl]+H)⁺: 507.5

### Example 65: 6-(4-(1-(4-chloro-3-fluorophenyl)-3-isobutyl-1H-pyrazolo[4,3-b]pyridine-5-carbonyl)-3,3-dimethylpiperazin-1-yl)-2,4-dimethylnicotinic acid

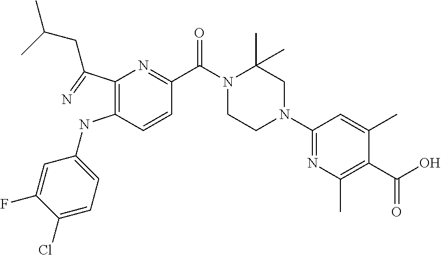 [SMILES: Cc1cc(N2CCN(C(=O)c3ccc(Nc4ccc(Cl)c(F)c4)c(C(=N)CC(C)C)n3)C(C)(C)C2)nc(C)c1C(=O)O]

Example 65 was prepared according to general procedure (XI) starting from Example 64 (150 mg). The crude was purified by flash chromatography (Merck 60®, DCM 100% to DCM/MeOH 80:20) then by preparative HPLC (column B, 5% to 100% ACN/H₂O), to obtain Example 65 (55 mg, 39%) as a white solid.

¹H-NMR (DMSO-d₆, 300 MHz) δ: 1.01 (d, J 6.6 Hz, 6H, 2 CH—CH₃); 1.54 (s, 6H, 2N—C—CH₃); 2.22-2.35 (m, 1H, CH—CH₃); 2.26 (s, 3H, CH₃); 2.38 (s, 3H, CH₃); 2.96 (d, J 7.0 Hz, 2H, CH₂—CH); 3.55 (bs, 2H, N—CH₂); 3.79 (bs, 2H, N—CH₂); 3.92 (s, 2H, N—CH₂); 6.32 (s, 1H, Ar); 7.68-7.84 (m, 3H, Ar); 7.91 (dd, J 10.5, 2.4 Hz, 1H, Ar); 8.50 (d, J 8.8 Hz, 1H, Ar); 12.70 (bs, 1H, COOH).

M/Z (M[³⁵Cl]+H)⁺: 593.5.

Mp: 215-220° C.

### Compound 116: 3-isobutyl-1-(6-(trifluoromethyl)pyridin-3-yl)-1H-pyrazolo[4,3-b]pyridine

Hydrazone was prepared according to general procedure (XXVa) starting from Compound 108 (460 mg) and 5-hydrazineyl-2-(trifluoromethyl)pyridine hydrochloride (540 mg, 1.2 equiv). The crude was directly cyclised according to general procedure (XXVII). The crude was purified by flash chromatography (Merck 60®, CyHex 100% to CyHex/EtOAc 80:20) to obtain Compound 116 (320 mg, 40%) as a yellow oil.

M/Z (M+H)⁺: 321.2

### Compound 117: 3-isobutyl-1-(6-(trifluoromethyl)pyridin-3-yl)-1H-pyrazolo[4,3-b]pyridine-4-oxide

Compound 117 was prepared according to general procedure (XIXb) starting from Compound 116 (320 mg). The crude residue was purified by flash chromatography (Merck 60®, CyHex 100% to EtOAc 100%) to obtain Compound 117 (300 mg, 91%) as a white solid.

M/Z (M+H)⁺: 337.2

### Compound 118: 3-isobutyl-1-(6-(trifluoromethyl)pyridin-3-yl)-1H-pyrazolo[4,3-b]pyridine-5-carbonitrile

Compound 118 was prepared according to general procedure (XX) starting from Compound 117 (400 mg). The crude residue was purified by flash chromatography (Merck 60®, CyHex 100% to CyHex/EtOAc 40:60) to obtain Compound 118 (200 mg, 65%) as a white solid.

M/Z (M+H)⁺: 346.2

### Compound 119: 3-isobutyl-1-(6-(trifluoromethyl)pyridin-3-yl)-1H-pyrazolo[4,3-b]pyridine-5-carboxamide

Compound 119 was obtained (200 mg, 93%) as a white solid according to general procedure (VII) starting from Compound 118 (200 mg).

M/Z (M+H)⁺: 364.3

### Compound 120: methyl 3-isobutyl-1-(6-(trifluoromethyl)pyridin-3-yl)-1H-pyrazolo[4,3-b]pyridine-5-carboxylate

Compound 120 was prepared according to general procedure (VIII) starting from Compound 119 (200 mg). The crude was purified by flash chromatography (Merck 60®, CyHex 100% to EtOAc 100%) to obtain Compound 120 (200 mg, 96%) as a white solid.

M/Z (M+H)⁺: 379.3

### Compound 121: 3-isobutyl-1-(6-(trifluoromethyl)pyridin-3-yl)-1H-pyrazolo[4,3-b]pyridine-5-carboxylic acid

Compound 121 was obtained (120 mg, 86%) as a white solid according to general procedure (IXa) starting from Compound 120 (200 mg).

M/Z (M+H)⁺: 365.2

### Example 66: 4-(3-isobutyl-1-(6-(trifluoromethyl)pyridin-3-yl)-1H-pyrazolo[4,3-b]pyridine-5-carbonyl)-3,3-dimethylpiperazin-2-one

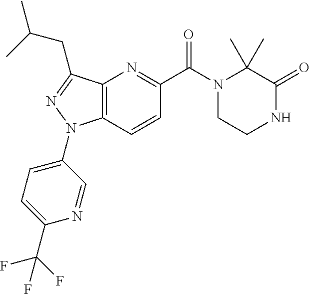 [SMILES: CC(C)Cc1nn(-c2ccc(C(F)(F)F)nc2)c2ccc(C(=O)N3CCNC(=O)C3(C)C)nc12]

Example 66 was prepared according to general procedure (Xa) starting from Compound 121 (70 mg) and 3,3-dimethylpiperazin-2-one (49 mg, 2 equiv), and using N,N-diisopropylethylamine (3.0 equiv). The crude was purified by flash chromatography (Merck 60®, CyHex 100% to EtOAc 100%), to obtain Example 66 (40 mg, 44%) as a white solid.

¹H-NMR (DMSO-d₆, 300 MHz) δ: 0.98 (d, J 6.7 Hz, 6H, 2 CH—CH₃); 1.72 (s, 6H, 2 C—CH₃); 2.23-2.27 (m, 1H, CH—CH₃); 2.97 (d, J 7.0 Hz, 2H, CH₂—CH); 3.51 (bs, 2H, N—CH₂); 7.78 (d, J 8.8 Hz, 1H, Ar); 8.11 (d, J 8.6 Hz, 1H, Ar); 8.13 (s, 1H, NH); 8.53 (dd, J 8.3, 2.2 Hz, 1H, Ar); 8.62 (d, J 8.8 Hz, 1H, Ar); 9.30 (d, J 2.5 Hz, 1H, Ar).

M/Z (M+H)⁺: 475.42.

Mp: 185° C.-190° C.

### Compound 122: 1-(3-chlorophenyl)-3-isobutyl-1H-pyrazolo[4,3-b]pyridine

Hydrazone was prepared according to general procedure (XXVa) starting from Compound 108 (300 mg) and (3-chlorophenyl)hydrazine hydrochloride (352 mg, 1.5 equiv). The crude was directly cyclised according to general procedure (XXVII). The crude was purified by flash chromatography (Merck 60®, CyHex 100% to CyHex/EtOAc 80:20) to obtain Compound 122 (250 mg, 53%) as a yellow oil.

M/Z (M[³⁵Cl]+H)⁺: 286.2

### Compound 123: 1-(3-chlorophenyl)-3-isobutyl-1H-pyrazolo[4,3-b]pyridine-4-oxide

Compound 123 was prepared according to general procedure (XIXb) starting from Compound 122 (250 mg). The crude residue was purified by flash chromatography (Merck 60®, DCM 100% to DCM/MeOH 80:20) to obtain Compound 123 (200 mg, 76%) as an off-white solid.

M/Z (M[³⁵Cl]+H)⁺: 302.3

### Compound 124: 1-(3-chlorophenyl)-3-isobutyl-1H-pyrazolo[4,3-b]pyridine-5-carbonitrile

Compound 124 was prepared according to general procedure (XX) starting from Compound 123 (200 mg). The crude residue was purified by flash chromatography (Merck 60®, CyHex 100% to CyHex/EtOAc 40:60) to obtain Compound 124 (190 mg, 93%) as a clear oil.

M/Z (M[³⁵Cl]+H)⁺: 311.3

### Compound 125: 1-(3-chlorophenyl)-3-isobutyl-1H-pyrazolo[4,3-b]pyridine-5-carboxamide

Compound 125 was obtained (200 mg, n.d.) as a white solid according to general procedure (VII) starting from Compound 124 (190 mg).

M/Z (M[³⁵Cl]+H)⁺: 329.3

### Compound 126: methyl 1-(3-chlorophenyl)-3-isobutyl-1H-pyrazolo[4,3-b]pyridine-5-carboxylate

Compound 126 was prepared according to general procedure (VIII) starting from Compound 125 (200 mg). The crude was purified by flash chromatography (Merck 60®, CyHex 100% to EtOAc 100%) to obtain Compound 125 (210 mg, n.d.) as a yellow oil.

M/Z (M[³⁵Cl]+H)⁺: 344.2

### Compound 127: 1-(3-chlorophenyl)-3-isobutyl-1H-pyrazolo[4,3-b]pyridine-5-carboxylic acid

Compound 127 was obtained (190 mg, n.d.) as a white solid according to general procedure (IXa) starting from Compound 126 (210 mg).

M/Z (M[³⁵Cl]+H)⁺: 330.2

### Example 67: 4-(1-(3-chlorophenyl)-3-isobutyl-1H-pyrazolo[4,3-b]pyridine-5-carbonyl)-3,3-dimethylpiperazin-2-one

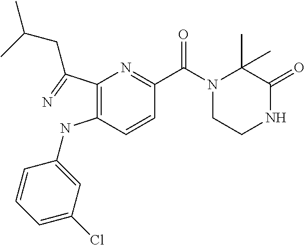 [SMILES: CC(C)CC(=N)c1nc(C(=O)N2CCNC(=O)C2(C)C)ccc1Nc1cccc(Cl)c1]

Example 67 was prepared according to general procedure (Xa) starting from Compound 127 (60 mg) and 3,3-dimethylpiperazin-2-one (47 mg, 2 equiv), and using N,N-diisopropylethylamine (3.0 equiv). The crude was purified by flash chromatography (Merck 60®, CyHex 100% to EtOAc 100%) then by preparative HPLC (column B, 5% to 100% ACN/H₂O), to obtain Example 67 (50 mg, 63%) as a white solid.

¹H-NMR (DMSO-d₆, 300 MHz) δ: 0.98 (d, J 6.6 Hz, 6H, 2 CH—CH₃); 1.72 (s, 6H, 2 CH₃); 2.18-2.34 (m, 1H, CH—CH₃); 2.94 (d, J 7.0 Hz, 2H, CH₂—CH); 3.52 (s, 2H, N—CH₂); 7.48 (d, J 8.0 Hz, 1H, Ar); 7.62 (t, J 8.0, 1H, Ar); 7.73 (d, J 8.8 Hz, 1H, Ar); 7.86 (s, 1H, Ar); 8.13 (s, 1H, NH); 8.45 (d, J 8.8 Hz, 1H, Ar); one N—CH₂ signal not observed.

M/Z (M[³⁵Cl]+H)⁺: 440.3.

Mp: 195° C.-200° C.

### Example 68: methyl 1-(1-(4-chloro-3-fluorophenyl)-3-isobutyl-1H-pyrazolo[4,3-b]pyridine-5-carbonyl)-3,3-dimethylpiperidine-4-carboxylate

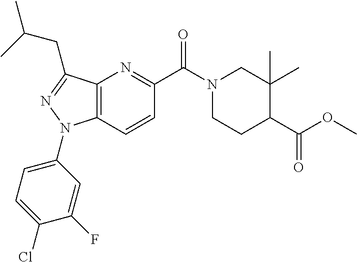 [SMILES: COC(=O)C1CCN(C(=O)c2ccc3c(n2)c(CC(C)C)nn3-c2ccc(Cl)c(F)c2)CC1(C)C]

Example 68 was prepared according to general procedure (Xa) starting from Compound 115 (45 mg) and 3,3-methyl 3,3-dimethylpiperidine-4-carboxylate (54 mg, 2 equiv), and using N,N-diisopropylethylamine (3.0 equiv). The crude was purified by flash chromatography (Merck 60®, CyHex 100% to EtOAc 100%), to obtain Example 68 (50 mg, 77%) as a clear oil.

M/Z (M[³⁵Cl]+H)⁺: 501.2

### Example 69: 1-(1-(4-chloro-3-fluorophenyl)-3-isobutyl-1H-pyrazolo[4,3-b]pyridine-5-carbonyl)-3,3-dimethylpiperidine-4-carboxylic acid

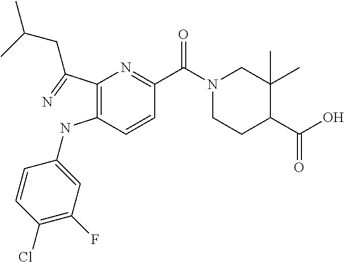 [SMILES: CC(C)CC(=N)c1nc(C(=O)N2CCC(C(=O)O)C(C)(C)C2)ccc1Nc1ccc(Cl)c(F)c1]

To a solution of Example 68 (50 mg) in THF (1 mL) was added lithium hydroxide 1M aq. (1.5 equiv). The reaction was stirred at 50° C. for 6 hours. The reaction mixture was concentrated. The crude was purified by preparative HPLC (column B, 5% to 100% ACN/H₂O), to obtain Example 69 (23 mg, 51%) as a white solid.

¹H-NMR (DMSO-d₆, 300 MHz) δ: 0.81-1.14 (m, 12H, 2 CH—CH₃+2 C—CH₃); 1.56-2.06 (m, 2H, N—CH₂—CH₂); 2.18-2.45 (m, 2H, CH—CH₃+CH—COOH); 2.75-3.18 (m, 4H, CH₂—CH+N—CH₂—C); 3.63 (d, J 12.9 Hz, 0.5H, one conformer of N—CH₂—CH₂); 3.81 (d, J 13.2 Hz, 0.5H, other conformer of N—CH₂—CH₂); 4.13 (d, J 12.6 Hz, 0.5H, one conformer of N—CH₂—CH₂); 4.47 (d, J 12.5 Hz, 0.5H, other conformer of N—CH₂—CH₂); 7.67-7.85 (m, 3H, Ar); 7.91 (dd, J 10.5, 2.4 Hz, 1H, Ar); 8.49 (dd, J 8.8, 2.2 Hz, 1H, Ar); 12.27 (bs, 1H, COOH).

M/Z (M[³⁵Cl]+H)⁺: 487.4.

Mp: 115-120° C.

### Example 70: 8-(1-(4-chloro-3-fluorophenyl)-3-isobutyl-1H-pyrazolo[4,3-b]pyridine-5-carbonyl)-1,3,8-triazaspiro[4.5]decane-2,4-dione

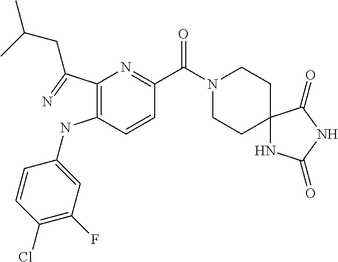 [SMILES: CC(C)CC(=N)c1nc(C(=O)N2CCC3(CC2)NC(=O)NC3=O)ccc1Nc1ccc(Cl)c(F)c1]

Example 70 was prepared according to general procedure (Xa) starting from Compound 115 (45 mg) and 1,3,8-triazaspiro[4.5]decane-2,4-dione (58 mg, 2 equiv), and using N,N-diisopropylethylamine (3.0 equiv). The crude was purified by preparative HPLC (column B, 5% to 100% ACN/H₂O), to obtain Example 70 (17 mg, 18%) as a white solid.

¹H-NMR (DMSO-d₆, 300 MHz) δ: 0.97 (d, J 6.6 Hz, 6H, 2 CH—CH₃); 1.61 (d, J 13.1 Hz, 1H, N—CH₂—CH₂); 1.72 (d, J 13.4 Hz, 1H, N—CH₂—CH₂); 1.83-2.02 (m, 2H, N—CH₂—CH₂); 2.20-2.32 (m, 1H, CH); 2.93 (d, J 6.2 Hz, 2H, CH₂—CH); 3.35-3.51 (m, 2H, N—CH₂); 3.85 (d, J 13.6 Hz, 1H, N—CH₂); 4.35 (d, J 13.2 Hz, 1H, N—CH₂); 7.69-7.85 (m, 3H, Ar); 7.92 (dd, J 10.5, 2.4 Hz, 1H, Ar); 8.51 (d, J 8.8 Hz, 1H, Ar); 8.63 (s, 1H, NH); 10.76 (s, 1H, NH).

M/Z (M[³⁵Cl]+H)⁺: 499.3.

Mp: 245-250° C.

### Compound 128: 1-(3-fluoropyridin-2-yl)ethan-1-one

Compound 128 was prepared according to general procedure (XXIVb) starting from 2-bromo-3-fluoropyridine (1.0 g) and N-methoxy-N-methylacetamide (1.29 g, 2.2 equiv). The crude residue was purified by flash chromatography (Merck 60®, CyHex 100% to CyHex/EtOAc 70:30) to obtain Compound 128 (400 mg, 51%) as a yellow oil.

M/Z (M+H)⁺: 140.1

### Compound 129: 1-(4-chloro-3-fluorophenyl)-3-methyl-1H-pyrazolo[4,3-b]pyridine

Hydrazone was prepared according to general procedure (XXVb) starting from Compound 128 (200 mg) and (4-chloro-3-fluorophenyl)hydrazine hydrochloride (347 mg, 1.5 equiv). The crude was directly cyclised according to general procedure (XXVII). The crude was purified by flash chromatography (Merck 60®, CyHex 100% to CyHex/EtOAc 80:20) to obtain Compound 129 (300 mg, 80%) as a yellow solid.

M/Z (M[³⁵Cl]+H)⁺: 262.1

### Compound 130: 1-(4-chloro-3-fluorophenyl)-3-methyl-1H-pyrazolo[4,3-b]pyridine-4-oxide

Compound 130 was prepared according to general procedure (XIXb) starting from Compound 129 (300 mg). The crude residue was purified by flash chromatography (Merck 60®, DCM 100% to DCM/MeOH 80:20) to obtain Compound 130 (200 mg, 63%) as a white solid.

M/Z (M[³⁵Cl]+H)⁺: 278.2

### Compound 131: 1-(4-chloro-3-fluorophenyl)-3-methyl-1H-pyrazolo[4,3-b]pyridine-5-carbonitrile

Compound 131 was prepared according to general procedure (XX) starting from Compound 130 (200 mg). The crude residue was purified by flash chromatography (Merck 60®, CyHex 100% to CyHex/EtOAc 80:20) to obtain Compound 131 (200 mg, 97%) as a beige solid.

M/Z (M[³⁵Cl]+H)⁺: 287.2

### Compound 132: 1-(4-chloro-3-fluorophenyl)-3-methyl-1H-pyrazolo[4,3-b]pyridine-5-carboxamide

Compound 132 was obtained (337 mg, n.d.) as a white solid according to general procedure (VII) starting from Compound 131 (200 mg).

M/Z (M[³⁵Cl]+H)⁺: 305.1

### Compound 133: methyl 1-(4-chloro-3-fluorophenyl)-3-methyl-1H-pyrazolo[4,3-b]pyridine-5-carboxylate

Compound 133 was prepared according to general procedure (VIII) starting from Compound 132 (337 mg). The crude was purified by flash chromatography (Merck 60®, CyHex 100% to CyHex/EtOAc 50:50) to obtain Compound 133 (170 mg, 76%) as a white solid.

M/Z (M[³⁵Cl]+H)⁺: 320.2

### Compound 134: 1-(4-chloro-3-fluorophenyl)-3-methyl-1H-pyrazolo[4,3-b]pyridine-5-carboxylic acid

Compound 134 was obtained (147 mg, 91%) as a white solid according to general procedure (IXa) starting from Compound 133 (170 mg).

M/Z (M[³⁵Cl]+H)⁺: 306.2

### Example 71: 4-(1-(4-chloro-3-fluorophenyl)-3-methyl-1H-pyrazolo[4,3-b]pyridine-5-carbonyl)-3,3-dimethylpiperazin-2-one

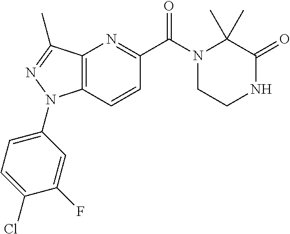 [SMILES: Cc1nn(-c2ccc(Cl)c(F)c2)c2ccc(C(=O)N3CCNC(=O)C3(C)C)nc12]

Example 71 was prepared according to general procedure (Xa) starting from Compound 134 (70 mg) and 3,3-dimethylpiperazin-2-one (59 mg, 2 equiv), and using N,N-diisopropylethylamine (3.0 equiv). The crude was purified by flash chromatography (Merck 60®, CyHex 100% to EtOAc 100%). The residue was triturated in Et₂O, the slurry was filtered to obtain Example 71 (52 mg, 55%) as a white solid.

¹H-NMR (DMSO-d₆, 300 MHz) δ: 1.71 (s, 6H, 2 C—CH₃); 2.62 (s, 3H, CH₃); 3.43-3.51 (m, 2H, N—CH₂); 7.72 (d, J 8.8 Hz, 2H, Ar); 7.76-7.82 (m, 1H, Ar); 7.90 (dd, J 10.5, 2.4 Hz, 1H, Ar); 8.12 (bs, 1H, NH); 8.48 (d, J 8.8 Hz, 1H, Ar). one N—CH₂ signal not observed.

M/Z (M[³⁵Cl]+H)⁺: 416.3.

Mp: 242-245° C.

### Compound 135: (3-fluoropyridin-2-yl)(tetrahydro-2H-pyran-4-yl)methanone

Compound 135 was prepared according to general procedure (XXIVb) starting from 2-bromo-3-fluoropyridine (0.51 g) and N-methoxy-N-methyltetrahydro-2H-pyran-4-carboxamide (1.0 g, 2.0 equiv). The crude residue was purified by flash chromatography (Merck 60®, EtOAc 100% to EtOAc/MeOH 80:20) to obtain Compound 135 (294 mg, 48%) as a yellow oil.

M/Z (M+H)⁺: 210.2

### Compound 136: 1-(4-chloro-3-fluorophenyl)-3-(tetrahydro-2H-pyran-4-yl)-1H-pyrazolo[4,3-b]pyridine

Hydrazone was prepared according to general procedure (XXVb) starting from Compound 135 (294 mg) and (4-chloro-3-fluorophenyl)hydrazine hydrochloride (331 mg, 1.2 equiv). The crude was directly cyclised according to general procedure (XXVII). The crude was purified by flash chromatography (Merck 60®, CyHex 100% to CyHex/EtOAc 80:20) to obtain Compound 136 (220 mg, 47%) as a yellow solid.

M/Z (M[³⁵Cl]+H)⁺: 332.1

### Compound 137: 1-(4-chloro-3-fluorophenyl)-3-(tetrahydro-2H-pyran-4-yl)-1H-pyrazolo[4,3-b]pyridine-4-oxide

Compound 137 was prepared according to general procedure (XIXb) starting from Compound 136 (220 mg). The crude residue was purified by flash chromatography (Merck 60®, EtOAc 100% to EtOAc/MeOH 80:20) to obtain Compound 137 (160 mg, 70%) as an off white solid.

M/Z (M[³⁵Cl]+H)⁺: 348.2

### Compound 138: 1-(4-chloro-3-fluorophenyl)-3-(tetrahydro-2H-pyran-4-yl)-1H-pyrazolo[4,3-b]pyridine-5-carbonitrile

Compound 138 was prepared according to general procedure (XX) starting from Compound 137 (160 mg). The crude residue was purified by flash chromatography (Merck 60®, CyHex 100% to CyHex/EtOAc 50:50) to obtain Compound 137 (90 mg, 55%) as a white solid.

M/Z (M[³⁵Cl]+H)⁺: 357.2

### Compound 139: 1-(4-chloro-3-fluorophenyl)-3-(tetrahydro-2H-pyran-4-yl)-1H-pyrazolo[4,3-b]pyridine-5-carboxamide

Compound 139 was obtained (90 mg, 95%) as a white solid according to general procedure (VII) starting from Compound 138 (90 mg).

M/Z (M[³⁵Cl]+H)⁺: 375.2

### Compound 140: methyl 1-(4-chloro-3-fluorophenyl)-3-(tetrahydro-2H-pyran-4-yl)-1H-pyrazolo[4,3-b]pyridine-5-carboxylate

Compound 140 was prepared according to general procedure (VIII) starting from Compound 139 (90 mg). The crude was purified by flash chromatography (Merck 60®, CyHex 100% to CyHex/EtOAc 50:50) to obtain Compound 140 (90 mg, 96%) as a white solid.

M/Z (M[³⁵Cl]+H)⁺: 390.3

### Compound 141: 1-(4-chloro-3-fluorophenyl)-3-(tetrahydro-2H-pyran-4-yl)-1H-pyrazolo[4,3-b]pyridine-5-carboxylic acid

Compound 141 was obtained (83 mg, 96%) as a white solid according to general procedure (IXa) starting from Compound 140 (90 mg).

M/Z (M[³⁵Cl]+H)⁺: 376.2

### Example 72: 4-(1-(4-chloro-3-fluorophenyl)-3-(tetrahydro-2H-pyran-4-yl)-1H-pyrazolo[4,3-b]pyridine-5-carbonyl)-3,3-dimethylpiperazin-2-one

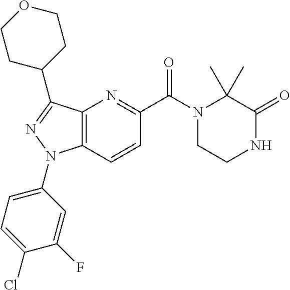 [SMILES: CC1(C)C(=O)NCCN1C(=O)c1ccc2c(n1)c(C1CCOCC1)nn2-c1ccc(Cl)c(F)c1]

Example 72 was prepared according to general procedure (Xa) starting from Compound 141 (80 mg) and 3,3-dimethylpiperazin-2-one (55 mg, 2 equiv), and using N,N-diisopropylethylamine (3.0 equiv). The crude was purified by flash chromatography (Merck 60®, EtOAc 100% to EtOAc/MeOH 90:10). The residue was triturated in Et₂O, the slurry was filtered to obtain Example 72 (38 mg, 37%) as a white solid.

¹H-NMR (DMSO-d₆, 300 MHz) δ: 1.72 (s, 6H, 2 C—CH₃); 2.02-2.13 (m, 4H, 2 O—CH₂—CH₂); 3.49-3.60 (m, 5H, 2 O—CH₂+CH); 3.96-4.01 (m, 2H, N—CH₂); 7.70-7.83 (m, 3H, Ar); 7.76-7.82 (m, 1H, Ar); 7.92 (dd, J 10.5, 2.4 Hz, 1H, Ar); 8.13 (bs, 1H, NH); 8.49 (d, J 8.9 Hz, 1H, Ar). one N—CH₂ signal not observed.

M/Z (M[³⁵Cl]+H)⁺: 486.4.

Mp: 286-290° C.

### Compound 142: 1-(2,4-dichlorophenyl)-3-isobutyl-1H-pyrazolo[4,3-b]pyridine

Hydrazone was prepared according to general procedure (XXVa) starting from Compound 108 (170 mg) and (2,4-dichlorophenyl)hydrazine hydrochloride (240 mg, 1.2 equiv). The crude was directly cyclised according to general procedure (XXVII). The crude was purified by flash chromatography (Merck 60®, CyHex 100% to CyHex/EtOAc 70:30) to obtain Compound 142 (200 mg, 42%) as a yellow oil.

M/Z (M[³⁵Cl₂]+H)⁺: 320.2

### Compound 143: 1-(2,4-dichlorophenyl)-3-isobutyl-1H-pyrazolo[4,3-b]pyridine-4-oxide

Compound 143 was prepared according to general procedure (XIXb) starting from Compound 142 (200 mg). The crude residue was purified by flash chromatography (Merck 60®, DCM 100% to DCM/MeOH 80:20) to obtain Compound 143 (120 mg, 58%) as a clear oil.

M/Z (M[³⁵Cl₂]+H)⁺: 336.3

### Compound 144: 1-(2,4-dichlorophenyl)-3-isobutyl-1H-pyrazolo[4,3-b]pyridine-5-carbonitrile

Compound 144 was prepared according to general procedure (XX) starting from Compound 143 (120 mg). The crude residue was purified by flash chromatography (Merck 60®, CyHex 100% to CyHex/EtOAc 70:30) to obtain Compound 144 (70 mg, 56%) as a clear oil.

M/Z (M[³⁵Cl₂]+H)⁺: 345.3

### Compound 145: 1-(2,4-dichlorophenyl)-3-isobutyl-1H-pyrazolo[4,3-b]pyridine-5-carboxamide

Compound 145 was obtained (105 mg, n.d.) as a white solid according to general procedure (VII) starting from Compound 144 (70 mg).

M/Z (M[³⁵Cl₂]+H)⁺: 363.2

### Compound 146: methyl 1-(2,4-dichlorophenyl)-3-isobutyl-1H-pyrazolo[4,3-b]pyridine-5-carboxylate

Compound 146 was prepared according to general procedure (VIII) starting from Compound 145 (100 mg). The crude was purified by flash chromatography (Merck 60®, CyHex 100% to CyHex/EtOAc 70:30) to obtain Compound 146 (75 mg, 97%) as a white solid.

M/Z (M[³⁵Cl₂]+H)⁺: 378.2

### Compound 147: 1-(2,4-dichlorophenyl)-3-isobutyl-1H-pyrazolo[4,3-b]pyridine-5-carboxylic acid

Compound 147 was obtained (75 mg, n.d.) as a white solid according to general procedure (IXa) starting from Compound 146 (75 mg).

M/Z (M[³⁵Cl₂]+H)⁺: 364.3

### Example 73: 4-(1-(2,4-dichlorophenyl)-3-isobutyl-1H-pyrazolo[4,3-b]pyridine-5-carbonyl)-3,3-dimethylpiperazin-2-one

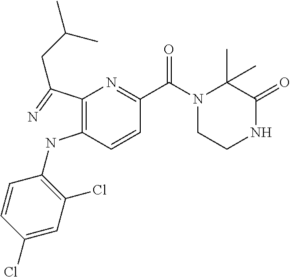 [SMILES: CC(C)CC(=N)c1nc(C(=O)N2CCNC(=O)C2(C)C)ccc1Nc1ccc(Cl)cc1Cl]

Example 73 was prepared according to general procedure (Xa) starting from Compound 147 (75 mg) and 3,3-dimethylpiperazin-2-one (51 mg, 2 equiv), and using N,N-diisopropylethylamine (3.0 equiv). The crude was purified by flash chromatography (Merck 60®, CyHex 100% to EtOAc 100%) The residue was taken up in water ACN mixture and freeze-dried to obtain Example 73 (66 mg, 70%) as a white solid.

¹H-NMR (DMSO-d₆, 300 MHz) δ: 0.97 (d, J 6.6 Hz, 6H, 2 CH—CH₃); 1.72 (s, 6H, 2 C—CH₃); 2.20-2.29 (m, 1H, CH—CH₃); 2.93 (d, J 7.0 Hz, 2H, CH₂—CH); 3.45-3.51 (m, 2H, N—CH₂); 7.65-7.73 (m, 3H, Ar); 7.90 (d, J 8.7 Hz, 1H, Ar); 7.99 (d, J 1.8 Hz, 1H, Ar); 8.10-8.13 (m, 1H, NH). one N—CH₂ signal not observed.

M/Z (M[³⁵Cl₂]+H)⁺: 474.3.

Mp: 205-210° C.

### Compound 148: 1-(2-chlorophenyl)-3-isobutyl-1H-pyrazolo[4,3-b]pyridine

Hydrazone was prepared according to general procedure (XXVa) starting from Compound 108 (400 mg) and (2-chlorophenyl)hydrazine hydrochloride (378 mg, 1.2 equiv). The crude was directly cyclised according to general procedure (XXVII). The crude was purified by flash chromatography (Merck 60®, CyHex 100% to CyHex/EtOAc 80:20) to obtain Compound 148 (420 mg, 67%) as a yellow oil.

M/Z (M[³⁵Cl]+H)⁺: 286.2

### Compound 149: 1-(2-chlorophenyl)-3-isobutyl-1H-pyrazolo[4,3-b]pyridine-4-oxide

Compound 149 was prepared according to general procedure (XIXb) starting from Compound 148 (420 mg). The crude residue was purified by flash chromatography (Merck 60®, DCM 100% to DCM/EtOAc 70:30) to obtain Compound 149 (320 mg, 72%) as a yellow solid.

M/Z (M[³⁵Cl]+H)⁺: 302.2

### Compound 150: 1-(2-chlorophenyl)-3-isobutyl-1H-pyrazolo[4,3-b]pyridine-5-carbonitrile

Compound 150 was prepared according to general procedure (XX) starting from Compound 149 (320 mg). The crude residue was purified by flash chromatography (Merck 60®, CyHex 100% to EtOAc 100%) to obtain Compound 150 (330 mg, 97%) as a clear oil.

M/Z (M[³⁵Cl]+H)⁺: 311.3

### Compound 151: 1-(2-chlorophenyl)-3-isobutyl-1H-pyrazolo[4,3-b]pyridine-5-carboxamide

Compound 151 was obtained (282 mg, n.d.) as a white solid according to general procedure (VII) starting from Compound 150 (266 mg).

M/Z (M[³⁵Cl]+H)⁺: 329.2

### Compound 152: methyl 1-(2-chlorophenyl)-3-isobutyl-1H-pyrazolo[4,3-b]pyridine-5-carboxylate

Compound 152 was prepared according to general procedure (VIII) starting from Compound 151 (282 mg). The crude was purified by flash chromatography (Merck 60®, CyHex 100% to CyHex/EtOAc 40:60) to obtain Compound 152 (230 mg, n.d.) as a clear oil.

M/Z (M[³⁵Cl]+H)⁺: 344.2

### Compound 153: 1-(2-chlorophenyl)-3-isobutyl-1H-pyrazolo[4,3-b]pyridine-5-carboxylic acid

Compound 153 was obtained (155 mg, 76%) as a clear oil according to general procedure (IXa) starting from Compound 152 (230 mg).

M/Z (M[³⁵Cl]+H)⁺: 330.2

### Example 74: 4-(1-(2-chlorophenyl)-3-isobutyl-1H-pyrazolo[4,3-b]pyridine-5-carbonyl)-3,3-dimethylpiperazin-2-one

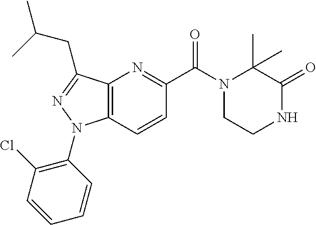 [SMILES: CC(C)Cc1nn(-c2ccccc2Cl)c2ccc(C(=O)N3CCNC(=O)C3(C)C)nc12]

Example 74 was prepared according to general procedure (Xa) starting from Compound 153 (70 mg) and 3,3-dimethylpiperazin-2-one (55 mg, 2 equiv), and using N,N-diisopropylethylamine (3.0 equiv). The crude was purified by flash chromatography (Merck 60®, CyHex 100% to EtOAc 100%) then the residue was triturated in Et₂O, to obtain Example 74 (50 mg, 54%) as a white solid.

¹H-NMR (DMSO-d₆, 300 MHz) δ: 0.97 (d, J 6.7 Hz, 6H, 2 CH—CH₃); 1.71 (s, 6H, 2 C—CH₃); 2.22-2.27 (m, 1H, CH—CH₃); 2.93 (d, J 7.1 Hz, 2H, CH₂—CH); 3.48-3.56 (m, 2H, N—CH₂); 7.58-7.68 (m, 4H, Ar); 7.76-7.80 (m, 1H, Ar); 7.83 (d, J 9.4 Hz, 1H, Ar); 8.11 (bs, 1H, NH). one N—CH₂ signal not observed.

M/Z (M[³⁵Cl]+H)⁺: 440.3.

Mp: 192° C.-196° C.

### Compound 154: 1-(3-fluoropyridin-2-yl)-2,2-dimethylpropan-1-ol

Under inert atmosphere, to a solution of 2-bromo-3-fluoropyridine (2.00 g) in THF (100 mL) at −78° C. was added a solution of ButylLithium 2.5 M in hexane (13.6 mL, 3.0 equiv). The reaction mixture was stirred at −78° C. for 1 hour then pivalaldehyde (1.23 mL, 1.0 equiv) was added and the reaction mixture was let warm up to 25° C. over 18 hours and quenched with NH₄Cl (sat. aq.) and stirred for 1 hour. The mixture was extracted with EtOAc, the organic layer was washed with brine, dried over magnesium sulfate then concentrated to dryness. The crude was purified by flash chromatography (Merck 60®, CyHex 100% to CyHex/EtOAc 70:30) to obtain Compound 154 (1.19 g, 53%) as a yellow oil.

M/Z (M+H)⁺: 184.0

### Compound 155: 1-(3-fluoropyridin-2-yl)-2,2-dimethylpropan-1-one

To a solution of Compound 154 (1.19 g) in DCM (500 mL) at 25° C. was added MnO₂ (5.64 g, 10.0 equiv). The reaction mixture was stirred for 18 hours then filtered over a pad of Celite. The filtrate was concentrated and was purified by flash chromatography (CyHex 100% to DCM 100%) to obtain Compound 155 (360 mg, 30%) as a clear oil.

M/Z (M+H)⁺: 184.0

### Compound 156: 3-(tert-butyl)-1-(4-chloro-3-fluorophenyl)-1H-pyrazolo[4,3-b]pyridine

Hydrazone was prepared according to general procedure (XXVa) starting from Compound 155 (360 mg) and (4-chloro-3-fluorophenyl)hydrazine hydrochloride (479 mg, 1.5 equiv). The crude was directly cyclised according to general procedure (XXVII). The crude was purified by flash chromatography (Merck 60®, CyHex 100% to CyHex/EtOAc 70:30) to obtain Compound 156 (220 mg, 11%) as a red oil.

M/Z (M[³⁵Cl]+H)⁺: 304.2

### Compound 157: 3-(tert-butyl)-1-(4-chloro-3-fluorophenyl)-1H-pyrazolo[4,3-b]pyridine-4-oxide

Compound 157 was prepared according to general procedure (XIXb) starting from Compound 156 (220 mg). The crude residue was purified by flash chromatography (Merck 60®, DCM 100% to EtOAc 100%) to obtain Compound 157 (90 mg, 35%) as a brown solid.

M/Z (M[³⁵Cl]+H)⁺: 320.2

### Compound 158: 3-(tert-butyl)-1-(4-chloro-3-fluorophenyl)-1H-pyrazolo[4,3-b]pyridine-5-carbonitrile

Compound 158 was prepared according to general procedure (XX) starting from Compound 157 (90 mg). The crude residue was purified by flash chromatography (Merck 60®, CyHex 100% to CyHex/EtOAc 50:50) to obtain Compound 158 (91 mg, 98%) as a white solid.

M/Z (M[³⁵Cl]+H)⁺: 329.2

### Compound 159: 3-(tert-butyl)-1-(4-chloro-3-fluorophenyl)-1H-pyrazolo[4,3-b]pyridine-5-carboxamide

Compound 159 was obtained (140 mg, n.d.) as a white solid according to general procedure (VII) starting from Compound 158 (86 mg).

M/Z (M[³⁵Cl]+H)⁺: 347.3

### Compound 160: methyl 3-(tert-butyl)-1-(4-chloro-3-fluorophenyl)-1H-pyrazolo[4,3-b]pyridine-5-carboxylate

Compound 160 was prepared according to general procedure (VIII) starting from Compound 159 (140 mg). The crude was purified by flash chromatography (Merck 60®, CyHex 100% to CyHex/EtOAc 70:30) to obtain Compound 160 (77 mg, 81%) as a white solid.

M/Z (M[³⁵Cl]+H)⁺: 362.2

### Compound 161: 3-(tert-butyl)-1-(4-chloro-3-fluorophenyl)-1H-pyrazolo[4,3-b]pyridine-5-carboxylic acid

Compound 161 was obtained (66 mg, 89%) as a white solid according to general procedure (IXa) starting from Compound 160 (77 mg).

M/Z (M[³⁵Cl]+H)⁺: 348.1

### Example 75: 3-(tert-butyl)-1-(4-chloro-3-fluorophenyl)-1H-pyrazolo[4,3-b]pyridine-5-carbonyl)-3,3-dimethylpiperazin-2-one

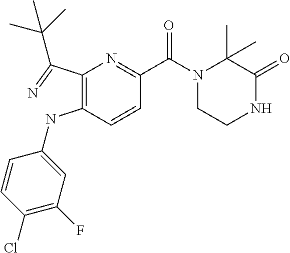 [SMILES: CC(C)(C)C(=N)c1nc(C(=O)N2CCNC(=O)C2(C)C)ccc1Nc1ccc(Cl)c(F)c1]

Example 75 was prepared according to general procedure (Xa) starting from Compound 161 (66 mg) and 3,3-dimethylpiperazin-2-one (48 mg, 2 equiv), and using N,N-diisopropylethylamine (3.0 equiv). The crude was purified by flash chromatography (Merck 60®, CyHex 100% to EtOAc 100%). The residue was triturated in Et2O, then the product was taken up in a water methanol mixture and freeze dried to obtain Example 75 (23 mg, 37%) as a white solid.

¹H-NMR (DMSO-d₆, 300 MHz) δ: 1.57 (s, 9H, C(CH₃)₃); 1.73 (s, 6H, 2 C—CH₃); 3.55-3.60 (m, 2H, N—CH₂); 7.71-7.83 (m, 3H, Ar); 7.91 (dd, J 2.4, 10.1 Hz, 1H, Ar); 8.10-8.16 (m, 1H, NH); 8.48 (d, J 8.9 Hz, 1H, Ar). One N—CH₂ signal not observed.

M/Z (M[³⁵Cl]+H)⁺: 458.3.

Mp: 236-238° C.

### Compound 162: cyclopentyl(3-fluoropyridin-2-yl)methanone

Compound 162 was prepared according to general procedure (XXIVb) starting from 2-bromo-3-fluoropyridine (1.40 g) and N-methoxy-N-methylcyclopentanecarboxamide (2.50 g, 2.0 equiv). The crude residue was purified by flash chromatography (Merck 60®, CyHex 100% to CyHex/EtOAc 80:20) to obtain Compound 162 (1.12 g, 73%) as a yellow oil.

M/Z (M+H)⁺: 182.2

### Compound 163: 1-(4-chloro-3-fluorophenyl)-3-cyclopentyl-1H-pyrazolo[4,3-b]pyridine

Hydrazone was prepared according to general procedure (XXVb) starting from Compound 162 (400 mg) and (4-chloro-3-fluorophenyl)hydrazine hydrochloride (400 mg, 1.2 equiv). The crude was directly cyclised according to general procedure (XXVII). The crude was purified by flash chromatography (Merck 60®, CyHex 100% to CyHex/EtOAc 80:20) to obtain Compound 163 (330 mg, 40%) as a white solid.

M/Z (M[³⁵Cl]+H)⁺: 316.2

### Compound 164: 1-(4-chloro-3-fluorophenyl)-3-cyclopentyl-1H-pyrazolo[4,3-b]pyridine-4-oxide

Compound 164 was prepared according to general procedure (XIXb) starting from Compound 163 (330 mg). The crude residue was purified by flash chromatography (Merck 60®, DCM 100% to DCM/MeOH 80:20) to obtain Compound 164 (150 mg, 43%) as an orange oil.

M/Z (M[³⁵Cl]+H)⁺: 332.3

### Compound 165: 1-(4-chloro-3-fluorophenyl)-3-cyclopentyl-1H-pyrazolo[4,3-b]pyridine-5-carbonitrile

Compound 165 was prepared according to general procedure (XX) starting from Compound 164 (150 mg). The crude residue was purified by flash chromatography (Merck 60®, CyHex 100% to EtOAc 100%) to obtain Compound 165 (100 mg, 62%) as a white solid.

M/Z (M[³⁵Cl]+H)⁺: 341.1

### Compound 166: 1-(4-chloro-3-fluorophenyl)-3-cyclopentyl-1H-pyrazolo[4,3-b]pyridine-5-carboxamide

Compound 166 was obtained (145 mg, n.d.) as a white solid according to general procedure (VII) starting from Compound 165 (100 mg).

M/Z (M[³⁵Cl]+H)⁺: 359.3

### Compound 167: methyl 1-(4-chloro-3-fluorophenyl)-3-cyclopentyl-1H-pyrazolo[4,3-b]pyridine-5-carboxylate

Compound 167 was prepared according to general procedure (VIII) starting from Compound 166 (145 mg). The crude was purified by flash chromatography (Merck 60®, CyHex 100% to CyHex/EtOAc 50:50) to obtain Compound 167 (83 mg, 75%) as a white solid.

M/Z (M[³⁵Cl]+H)⁺: 374.3

### Compound 168: 1-(4-chloro-3-fluorophenyl)-3-cyclopentyl-1H-pyrazolo[4,3-b]pyridine-5-carboxylic acid

Compound 168 was obtained (84 mg, n.d.) as a white solid according to general procedure (IXa) starting from Compound 167 (83 mg).

M/Z (M[³⁵Cl]+H)⁺: 360.1

### Example 76: 4-(1-(4-chloro-3-fluorophenyl)-3-cyclopentyl-1H-pyrazolo[4,3-b]pyridine-5-carbonyl)-3,3-dimethylpiperazin-2-one

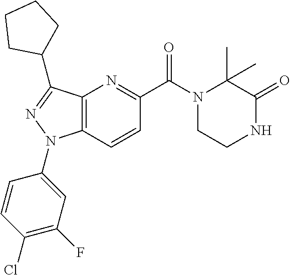 [SMILES: CC1(C)C(=O)NCCN1C(=O)c1ccc2c(n1)c(C1CCCC1)nn2-c1ccc(Cl)c(F)c1]

Example 76 was prepared according to general procedure (Xa) starting from Compound 168 (84 mg) and 3,3-dimethylpiperazin-2-one (60 mg, 2 equiv), and using N,N-diisopropylethylamine (3.0 equiv). The crude was purified by flash chromatography (Merck 60®, CyHex 100% to EtOAc 100%). The residue was triturated in ACN, then the product was taken up in a water ACN mixture and freeze dried to obtain Example 76 (64 mg, 59%) as a white solid.

¹H-NMR (DMSO-d₆, 300 MHz) δ: 1.68-1.90 (m, 10H, 2 C—CH₃+2 CH—CH₂—CH₂); 2.05-2.17 (m, 4H, 2 CH—CH₂—CH₂); 3.50-3.54 (m, 2H, N—CH₂); 3.57-3.65 (m, 1H, CH); 7.70-7.82 (3H, Ar); 7.90 (dd, J 2.4, 10.5 Hz, 1H, Ar); 8.10-8.13 (m, 1H, NH); 8.48 (d, J 8.8 Hz, 1H, Ar). One N—CH₂ signal not observed.

M/Z (M[³⁵Cl]+H)⁺: 470.2.

Mp: >250° C.

### Compound 169: cyclopropyl(3-fluoropyridin-2-yl)methanone

Compound 169 was prepared according to general procedure (XXIVb) starting from 2-bromo-3-fluoropyridine (1.70 g) and N-methoxy-N-methylcyclopropanecarboxamide (2.50 g, 2.0 equiv). The crude residue was purified by flash chromatography (Merck 60®, CyHex 100% to CyHex/EtOAc 80:20) to obtain Compound 169 (0.96 g, 60%) as a yellow oil.

M/Z (M+H)⁺: 166.0

### Compound 170: 1-(4-chloro-3-fluorophenyl)-3-cyclopropyl-1H-pyrazolo[4,3-b]pyridine

Hydrazone was prepared according to general procedure (XXVb) starting from Compound 169 (300 mg) and (4-chloro-3-fluorophenyl)hydrazine hydrochloride (350 mg, 1.2 equiv). The crude was directly cyclised according to general procedure (XXVII). The crude was purified by flash chromatography (Merck 60®, CyHex 100% to CyHex/EtOAc 80:20) to obtain Compound 170 (400 mg, 57%) as an orange solid.

M/Z (M[³⁵Cl]+H)⁺: 288.2

### Compound 171: 1-(4-chloro-3-fluorophenyl)-3-cyclopropyl-1H-pyrazolo[4,3-b]pyridine-4-oxide

Compound 171 was prepared according to general procedure (XIXb) starting from Compound 170 (400 mg). The crude residue was purified by flash chromatography (Merck 60®, DCM 100% to DCM/MeOH 80:20) to obtain Compound 171 (290 mg, 69%) as a beige solid.

M/Z (M[³⁵Cl]+H)⁺: 304.1

### Compound 172: 1-(4-chloro-3-fluorophenyl)-3-cyclopropyl-1H-pyrazolo[4,3-b]pyridine-5-carbonitrile

Compound 172 was prepared according to general procedure (XX) starting from Compound 171 (290 mg). The crude residue was purified by flash chromatography (Merck 60®, CyHex 100% to CyHex/EtOAc 80:20) to obtain Compound 172 (240 mg, 81%) as a white solid.

M/Z (M[³⁵Cl]+H)⁺: 313.3

### Compound 173: 1-(4-chloro-3-fluorophenyl)-3-cyclopropyl-1H-pyrazolo[4,3-b]pyridine-5-carboxamide

Compound 173 was obtained (230 mg, 91%) as a white solid according to general procedure (VII) starting from Compound 172 (240 mg).

M/Z (M[³⁵Cl]+H)⁺: 313.3

### Compound 174: methyl 1-(4-chloro-3-fluorophenyl)-3-cyclopropyl-1H-pyrazolo[4,3-b]pyridine-5-carboxylate

Compound 174 was prepared according to general procedure (VIII) starting from Compound 173 (230 mg). The crude was triturated in Et₂O and the solid was isolated by filtration to obtain Compound 174 (190 mg, 79%) as a white solid.

M/Z (M[³⁵Cl]+H)⁺: 346.2

### Compound 175: 1-(4-chloro-3-fluorophenyl)-3-cyclopropyl-1H-pyrazolo[4,3-b]pyridine-5-carboxylic acid

Compound 175 was obtained (165 mg, 91%) as a white solid according to general procedure (IXa) starting from Compound 174 (190 mg).

M/Z (M[³⁵Cl]+H)⁺: 332.2

### Example 77: 4-(1-(4-chloro-3-fluorophenyl)-3-cyclopropyl-1H-pyrazolo[4,3-b]pyridine-5-carbonyl)-3,3-dimethylpiperazin-2-one

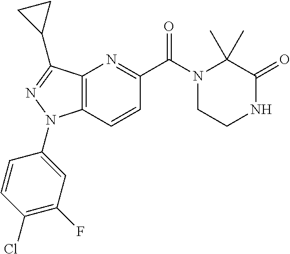 [SMILES: CC1(C)C(=O)NCCN1C(=O)c1ccc2c(n1)c(C1CC1)nn2-c1ccc(Cl)c(F)c1]

Example 77 was prepared according to general procedure (Xa) starting from Compound 175 (70 mg) and 3,3-dimethylpiperazin-2-one (54 mg, 2 equiv), and using N,N-diisopropylethylamine (3.0 equiv). The crude was purified by flash chromatography (Merck 60®, CyHex 100% to EtOAc 100%). The residue was triturated in Et₂O and the solid was isolated by filtration to obtain Example 77 (53 mg, 57%) as a white solid.

¹H-NMR (DMSO-d₆, 300 MHz) δ: 1.11-1.17 (m, 2H, c-Pr); 1.27-1.32 (m, 2H, c-Pr); 1.72 (s, 6H, 2 C—CH₃); 2.42-2.48 (m, 1H, CH c-Pr); 3.47-3.54 (m, 2H, N—CH₂); 7.76-7.81 (m, 3H, Ar); 7.88 (dd, J 10.5, 2.4 Hz, 1H, Ar); 8.13 (bs, 1H, NH); 8.46 (d, J 8.9 Hz, 1H, Ar). One N—CH₂ signal not observed.

M/Z (M[³⁵Cl]+H)⁺: 442.3

Mp: 228° C.-232° C.

### Compound 176: 3-isobutyl-1H-pyrazolo[4,3-b]pyridine

A suspension of Compound 108 (500 mg) in hydrazine hydrate (7 mL, 52 equiv) was subjected to microwave irradiation at 150° C. for 30 min. The reaction mixture was diluted with EtOAc and concentrated. The crude was purified by flash chromatography (Merck 60®, CyHex 100% to EtOAc 100%) to obtain Compound 176 (358 mg, 74%) as a white solid.

M/Z (M+H)⁺: 176.2

### Compound 177: 1-(4-chloro-3-fluorobenzyl)-3-isobutyl-1H-pyrazolo[4,3-b]pyridine

To a solution of Compound 176 (100 mg) in THF (6 mL) was added NaH 60% in mineral oil (46 mg, 2 equiv) at 0° C. The reaction mixture was stirred at 0° C. for 15 min then 4-(bromomethyl)-1-chloro-2-fluorobenzene (78 μL, 1 equiv) was added and the reaction mixture was stirred at 25° C. for 18 hours. The reaction mixture was quenched with water at 0° C. and the mixture was extracted with EtOAc, the organic layer was washed with brine, dried over magnesium sulfate then concentrated to dryness. The crude was purified by flash chromatography to afford hetroarylketone. The crude was purified by flash chromatography (Merck 60®, CyHex 100% to CyHex/EtOAc 50:50) to obtain Compound 177 (117 mg, 65%) as a white solid.

M/Z (M[³⁵Cl]+H)⁺: 318.2

### Compound 178: 1-(4-chloro-3-fluorobenzyl)-3-isobutyl-1H-pyrazolo[4,3-b]pyridine-4-oxide

Compound 178 was prepared according to general procedure (XIXb) starting from Compound 177 (117 mg). The crude residue Compound 178 (120 mg, n.d.) was obtained as a white solid.

M/Z (M[³⁵Cl]+H)⁺: 334.2

### Compound 179: 1-(4-chloro-3-fluorobenzyl)-3-isobutyl-1H-pyrazolo[4,3-b]pyridine-5-carbonitrile

Compound 179 was prepared according to general procedure (XX) starting from Compound 178 (120 mg). The crude residue was purified by flash chromatography (Merck 60®, CyHex 100% to CyHex/EtOAc 60:40) to obtain Compound 179 (102 mg, 83%) as a white solid.

M/Z (M[³⁵Cl]+H)⁺: 343.2

### Compound 180: 1-(4-chloro-3-fluorobenzyl)-3-isobutyl-1H-pyrazolo[4,3-b]pyridine-5-carboxamide

Compound 180 was obtained (100 mg, 93%) as a white solid according to general procedure (VII) starting from Compound 179 (102 mg).

M/Z (M[³⁵Cl]+H)⁺: 361.3

### Compound 181: methyl 1-(4-chloro-3-fluorobenzyl)-3-isobutyl-1H-pyrazolo[4,3-b]pyridine-5-carboxylate

Compound 181 was prepared according to general procedure (VIII) starting from Compound 180 (100 mg). The crude was purified by flash chromatography (Merck 60®, CyHex 100% to CyHex/EtOAc 50:50) to obtain Compound 181 (64 mg, 75%) as a white solid.

M/Z (M[³⁵Cl]+H)⁺: 376.2

### Compound 182: 1-(4-chloro-3-fluorobenzyl)-3-isobutyl-1H-pyrazolo[4,3-b]pyridine-5-carboxylic acid

Compound 182 was obtained (61 mg, 88%) as a white solid according to general procedure (IXa) starting from Compound 181 (64 mg).

M/Z (M[³⁵Cl]+H)⁺: 362.2

### Example 78: 4-(1-(4-chloro-3-fluorobenzyl)-3-isobutyl-1H-pyrazolo[4,3-b]pyridine-5-carbonyl)-3,3-dimethylpiperazin-2-one

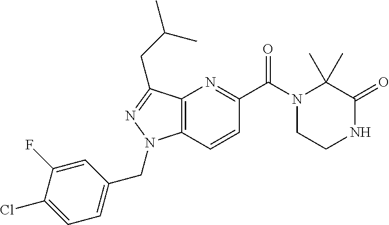 [SMILES: CC(C)Cc1nn(Cc2ccc(Cl)c(F)c2)c2ccc(C(=O)N3CCNC(=O)C3(C)C)nc12]

Example 78 was prepared according to general procedure (Xa) starting from Compound 182 (50 mg) and 3,3-dimethylpiperazin-2-one (60 mg, 2 equiv), and using N,N-diisopropylethylamine (3.0 equiv). The crude was purified by flash chromatography (Merck 60®, CyHex 100% to EtOAc 100%). The residue was recrystallized from a hot iPrOH/iPr₂O mixture to obtain Example 78 (25 mg, 38%) as a white solid.

¹H-NMR (DMSO-d₆, 300 MHz) δ: 0.90 (d, J 6.7 Hz, 6H, 2 CH—CH₃); 1.69 (s, 6H, 2 C—CH₃); 2.12-2.21 (m, 1H, CH—CH₃); 2.84 (d, J 7.1 Hz, 2H, CH₂—CH); 3.26-3.33 (m, 2H, N—CH₂); 3.42-3.50 (m, 2H, N—CH₂); 5.67 (s, 2H, Ar—CH₂); 7.03 (d, J 8.3 Hz, 1H, Ar); 7.29 (dd, J 10.2, 1.9 Hz, 1H, Ar); 7.53 (t, J 8.0 Hz, 1H, Ar); 7.62 (d, J 8.8 Hz, 1H, Ar); 8.08 (bs, 1H, NH); 8.26 (d, J 8.8 Hz, 1H, Ar).

M/Z (M[³⁵Cl]+H)⁺: 472.4.

Mp: 198-202° C.

### Compound 183: 3-isobutyl-1H-pyrazolo[4,3-b]pyridine 4-oxide

Compound 183 was prepared according to general procedure (XIXb) starting from Compound 177 (288 mg). The crude residue Compound 183 (239 mg, 76%) as a yellow oil.

M/Z (M+H)⁺: 192.1

### Compound 184: 3-isobutyl-1H-pyrazolo[4,3-b]pyridine-5-carbonitrile

Compound 184 was prepared according to general procedure (XX) starting from Compound 183 (230 mg). The crude residue was purified by flash chromatography (Merck 60®, CyHex 100% to CyHex/EtOAc 60:40) to obtain Compound 184 (204 mg, 85%) as a yellow oil.

M/Z (M+H)⁺: 201.1

### Compound 185: 3-isobutyl-1-(4-(trifluoromethoxy)phenyl)-1H-pyrazolo[4,3-b]pyridine-5-carbonitrile

Compound 185 was prepared according to general procedure (XXVIII) starting from Compound 184 (110 mg) and 4-trifluoro (4-(trifluoromethoxy)phenyl)boronic acid (226 mg, 2 equiv). The crude residue was purified by flash chromatography (Merck 60®, CyHex 100% to CyHex/EtOAc 70:30) to obtain Compound 185 (180 mg, n.d.) as a white solid.

M/Z (M+H)⁺: 361.2

### Compound 186: 3-isobutyl-1-(4-(trifluoromethoxy)phenyl)-1H-pyrazolo[4,3-b]pyridine-5-carboxamine

Compound 186 was obtained (157 mg, 77%) as a white solid according to general procedure (VII) starting from Compound 185 (180 mg).

M/Z (M+H)⁺: 379.2

### Compound 187: methyl 3-isobutyl-1-(4-(trifluoromethoxy)phenyl)-1H-pyrazolo[4,3-b]pyridine-5-carboxylate

Compound 187 was prepared according to general procedure (VIII) starting from Compound 186 (157 mg). The crude was purified by flash chromatography (Merck 60®, CyHex 100% to CyHex/EtOAc 70:30) to obtain Compound 187 (154 mg, 94%) as a white solid.

M/Z (M+H)⁺: 394.2

### Compound 188: 3-isobutyl-1-(4-(trifluoromethoxy)phenyl)-1H-pyrazolo[4,3-b]pyridine-5-carboxylic acid

Compound 188 was obtained (144 mg, 91%) as a white solid according to general procedure (IXa) starting from Compound 187 (154 mg).

M/Z (M+H)⁺: 380.2

### Example 79: 4-(3-isobutyl-1-(4-(trifluoromethoxy)phenyl)-1H-pyrazolo[4,3-b]pyridine-5-carbonyl)-3,3-dimethylpiperazin-2-one

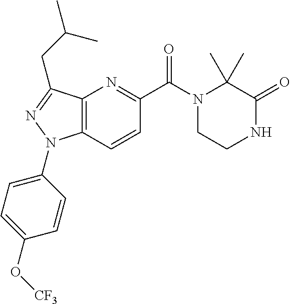 [SMILES: CC(C)Cc1nn(-c2ccc(OC(F)(F)F)cc2)c2ccc(C(=O)N3CCNC(=O)C3(C)C)nc12]

Example 79 was prepared according to general procedure (Xa) starting from Compound 188 (70 mg) and 3,3-dimethylpiperazin-2-one (47 mg, 2 equiv), and using N,N-diisopropylethylamine (3.0 equiv). The crude was purified by flash chromatography (Merck 60®, CyHex 100% to EtOAc 100%). The residue was triturated in Et₂O then was taken up in a water ACN mixture and freeze dried to obtain Example 79 (54 mg, 60%) as a white solid.

¹H-NMR (DMSO-d₆, 300 MHz) δ: 0.97 (d, J 6.7 Hz, 6H, CH—CH₃); 1.72 (s, 6H, 2 C—CH₃); 2.25-2.27 (m, 1H, CH—CH₃); 2.94 (d, J 7.0 Hz, 2H, CH₂—CH); 3.49-3.53 (m, 2H, N—CH₂); 7.59 (d, J 8.3 Hz, 2H, Ar); 7.72 (d, J 8.8 Hz, 1H, Ar); 7.93-7.96 (m, 2H, Ar); 8.12 (bs, 1H, NH); 8.43 (d, J 8.8 Hz, 1H, Ar). One N—CH₂ signal not observed.

M/Z (M+H)⁺: 490.3.

Mp: 185-187° C.

### Compound 189: ethyl 2-hydrazineylpyrimidine-5-carboxylate

To a solution of ethyl 2-chloropyrimidine-5-carboxylate (470 mg) in EtOH (5 mL) was added hydrazine hydrate (0.3 mL, 2.4 equiv). The reaction mixture was subjected to microwave irradiation at 50° C. for 10 min. The reaction mixture was diluted with water (10 mL) and extracted with DCM (3*5 mL), the organic layer was washed with brine, dried over magnesium sulfate then concentrated to dryness to obtain Compound 189 (440 mg, 96%) as a yellow solid.

M/Z (M+H)⁺: 182.9

### Compound 190: ethyl 3-(tert-butyl)-1H-pyrazolo[3,4-b]pyridine-5-carboxylate

To a solution of ethynyltrimethylsilane (0.5 mL) in THF (15 mL) was added butyllithium (1.6 M in hexanes, 2.2 mL, 1 equiv) at −78° C. The reaction mixture was stirred at −78° C. for 1 hour, then a solution of N-methoxy-N-methylpivalamide (510 mg, 1 equiv) in THF (2 mL) was added dropwise. The reaction mixture was let warm up at room temperature then was quenched with NH₄Cl (sat. aq., 10 mL) and extracted with DCM (3*10 mL). The organic layer was washed with brine, dried over magnesium sulfate then partially concentrated to obtain a solution of 4,4-dimethyl-1-(trimethylsilyl)pent-1-yn-3-one in THF (˜3 mL).

The solution was diluted with THF (12 mL) and Compound 189 (440 mg) was added followed by trifluoroacetic acid (10 μL, 0.1 equiv). The reaction mixture was heated at 50° C. for 1 hour, then concentrated under reduced pressure. The residue was taken up in THF (12 mL), pyridine (0.2 mL, 2 equiv) and trifluoroacetic anhydride (0.27 mL, 1.5 equiv) were added, and the reaction mixture was heated at 60° C. for 60 hours. The reaction mixture was concentrated to dryness and the crude was purified by flash chromatography (Interchim® 50 μm, CyHex 100% to CyHex/EtOAc 30:70) to obtain Compound 190 (220 mg, 25%) as a yellow oil.

M/Z (M+H)⁺: 248.1.

### Compound 191: ethyl 3-(tert-butyl)-1-(4-chloro-3-fluorophenyl)-1H-pyrazolo[3,4-b]pyridine-5-carboxylate

To a solution of Compound 190 (110 mg) in DCM (0.02 M) was added (4-chloro-3-fluorophenyl)boronic acid (357 mg, 4.6 equiv), Cu(OAc)₂ (472 mg, 4.6 equiv), and pyridine (0.18 mL, 5.0 equiv). The reaction mixture was stirred at room temperature for 20 hours then concentrated. The crude residue was purified by flash chromatography (Interchim® 50 μm, CyHex 100% to CyHex/EtOAc 80:20) to obtain Compound 191 (101 mg, 60%) as a white solid.

M/Z (M[³⁵Cl]+H)⁺: 376.2.

### Compound 192: lithium 3-(tert-butyl)-1-(4-chloro-3-fluorophenyl)-1H-pyrazolo[3,4-b]pyridine-5-carboxylate

Compound 192 was obtained (100 mg, n.d.) as a yellow solid according to general procedure (IXb) starting from Compound 191 (101 mg).

M/Z (M[³⁵Cl]+H)⁺: 348.1

### Example 80: 4-(3-(tert-butyl)-1-(4-chloro-3-fluorophenyl)-1H-pyrazolo[3,4-b]pyridine-5-carbonyl)-3,3-dimethylpiperazin-2-one

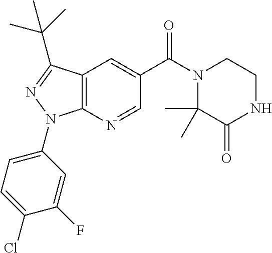 [SMILES: CC(C)(C)c1nn(-c2ccc(Cl)c(F)c2)c2ncc(C(=O)N3CCNC(=O)C3(C)C)cc12]

Example 80 was prepared according to general procedure (Xa) starting from Compound 192 (50 mg) and 3,3-dimethylpiperazin-2-one (22 mg, 1.2 equiv), and using N,N-diisopropylethylamine (3.0 equiv). The crude was purified by flash chromatography (Interchim® 50 μm, CyHex 100% to CyHex/EtOAc 75:25) to obtain Example 80 (10 mg, 15%) as a white solid.

¹H-NMR (DMSO-d₆, 400 MHz) δ: 1.54 (s, 9H, C(CH₃)₃); 1.75 (s, 6H, 2 C—CH₃); 3.26-3.29 (m, 2H, N—CH₂); 3.44-3.51 (m, 2H, N—CH₂); 7.80 (t, J 8.6 Hz, 1H, Ar); 8.11 (bs, 1H, NH); 8.23 (dd, J 8.6, 2.3 Hz, 1H, Ar); 8.40 (dd, J 11.2, 2.3 Hz, 1H, Ar); 8.64 (d, J 2.0 Hz, 1H, Ar), 8.73 (d, J 2.0 Hz, 1H, Ar).

M/Z (M[³⁵Cl]+H)⁺: 458.3.

Mp: >220° C.

### Compound 193: 1-(3-fluoro-5-methylpyridin-2-yl)-3-methylbutan-1-ol

Under inert atmosphere, to a solution of 2-bromo-3-fluoro-5-methylpyridine (1.04 g) in THF (40 mL) at −78° C. was added a solution of ButylLithium 2.5 M in hexane (6.56 mL, 3.0 equiv). The reaction mixture was stirred at −78° C. for 60 minutes then isovaleraldehyde (0.70 mL, 1.2 equiv) was added and the reaction mixture was let warm up at room temperature for 6 hours, and quenched with NH₄Cl (sat. aq.). The mixture was extracted with EtOAc, the organic layer was washed with brine, dried over magnesium sulfate then concentrated to dryness. The crude was purified by flash chromatography (Merck 60®, CyHex 100% to EtOAc 100%) to obtain Compound 193 (410 mg, 38%) as a clear oil.

M/Z (M+H)⁺: 198.1.

### Compound 194: 1-(3-fluoro-5-methylpyridin-2-yl)-3-methylbutan-1-one

To a solution of Compound 193 (170 mg) in DCM (20 mL) was added manganese dioxide (750 mg, 10.0 equiv). The reaction mixture was stirred at 25° C. for 18 hours. The reaction mixture was filtered over Celite and the filtrate was concentrated under reduced pressure to obtain Compound 194 (140 mg, 83%) as a clear oil.

M/Z (M+H)⁺: 196.1.

### Compound 195: 1-(4-chloro-3-fluorophenyl)-3-isobutyl-6-methyl-1H-pyrazolo[4,3-b]pyridine

Hydrazone was prepared according to general procedure (XXVb) starting from Compound 194 (310 mg) and (4-chloro-3-fluorophenyl)hydrazine hydrochloride (350 mg, 1.2 equiv). The crude was directly cyclised according to general procedure (XXVII). The crude was purified by flash chromatography (Merck 60®, CyHex 100% to CyHex/EtOAc 70:30) to obtain Compound 195 (350 mg, 69%) as a yellow oil.

M/Z (M[³⁵Cl]+H)⁺: 318.3

### Compound 196: 1-(4-chloro-3-fluorophenyl)-3-isobutyl-6-methyl-1H-pyrazolo[4,3-b]pyridine-4-oxide

Compound 196 was prepared according to general procedure (XIXb) starting from Compound 195 (350 mg). The crude residue was purified by flash chromatography (Merck 60®, DCM 100% to DCM/MeOH 80:20) to obtain Compound 196 (300 mg, 82%) as a white solid.

M/Z (M[³⁵Cl]+H)⁺: 334.2

### Compound 197: 1-(4-chloro-3-fluorophenyl)-3-isobutyl-6-methyl-1H-pyrazolo[4,3-b]pyridine-5-carbonitrile

Compound 197 was prepared according to general procedure (XX) starting from Compound 196 (300 mg). The crude residue was purified by flash chromatography (Merck 60®, CyHex 100% to CyHex/EtOAc 70:30) to obtain Compound 197 (250 mg, 79%) as a white solid.

M/Z (M[³⁵Cl]+H)⁺: 343.2

### Compound 198: 1-(4-chloro-3-fluorophenyl)-3-isobutyl-6-methyl-1H-pyrazolo[4,3-b]pyridine-5-carboxamide

Compound 198 was obtained (540 mg, n.d.) as a clear oil according to general procedure (VII) starting from Compound 197 (245 mg).

M/Z (M[³⁵Cl]+H)⁺: 361.3

### Compound 199: methyl 1-(4-chloro-3-fluorophenyl)-3-isobutyl-6-methyl-1H-pyrazolo[4,3-b]pyridine-5-carboxylate

Compound 199 was prepared according to general procedure (VIII) starting from Compound 198 (540 mg). The crude was triturated in Et₂O and the solid was isolated by filtration to obtain Compound 199 (300 mg, n.d.) as a clear oil.

M/Z (M[³⁵Cl]+H)⁺: 376.2

### Compound 200: 1-(4-chloro-3-fluorophenyl)-3-isobutyl-6-methyl-1H-pyrazolo[4,3-b]pyridine-5-carboxylic acid

Compound 200 was obtained (100 mg, 34%) as a white solid according to general procedure (IXa) starting from Compound 199 (300 mg).

M/Z (M[³⁵Cl]+H)⁺: 362.1

### Example 81: 1-(4-chloro-3-fluorophenyl)-3-isobutyl-6-methyl-1H-pyrazolo[4,3-b]pyridine-5-carbonyl)-3,3-dimethylpiperazin-2-one

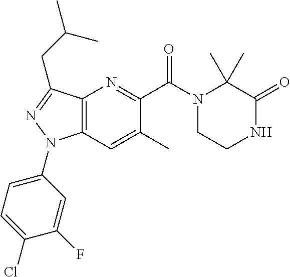 [SMILES: Cc1cc2c(nc1C(=O)N1CCNC(=O)C1(C)C)c(CC(C)C)nn2-c1ccc(Cl)c(F)c1]

Example 81 was prepared according to general procedure (Xa) starting from Compound 200 (50 mg) and 3,3-dimethylpiperazin-2-one (36 mg, 2 equiv), and using N,N-diisopropylethylamine (3.0 equiv). The crude was purified by flash chromatography (Merck 60®, CyHex 100% to EtOAc 100%) to obtain Example 81 (40 mg, 65%) as a white solid.

¹H-NMR (DMSO-d₆, 300 MHz) δ: 0.96 (d, J 7.1 Hz, 6H, CH—CH₃); 1.77 (s, 6H, 2 C(CH₃)₂); 2.15-2.31 (m, 1H, CH—CH₃); 2.43 (s, 3H, CH₃); 2.90 (d, J 6.4 Hz, 2H, CH₂—CH); 3.07-3.18 (m, 2H, N—CH₂); 3.18-3.25 (m, 2H, N—CH₂); 7.68-7.83 (m, 2H, Ar); 7.90 (dd, J 10.6, 2.8 Hz, 1H, Ar); 8.14 (bs, 1H, NH); 8.37 (s, 1H, Ar).

M/Z (M[³⁵Cl]+H)⁺: 472.3.

Mp: 213-215° C.

### Example 82: 4-(1-isobutyl-3-(piperidin-1-yl)-1H-pyrrolo[2,3-b]pyridine-6-carbonyl)-3,3-dimethylpiperazin-2-one

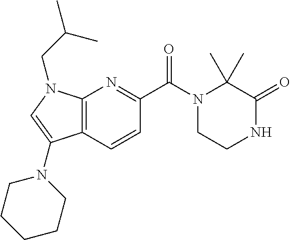 [SMILES: CC(C)Cn1cc(N2CCCCC2)c2ccc(C(=O)N3CCNC(=O)C3(C)C)nc21]

Under inert atmosphere, to a solution of Compound 99 (50 mg) in toluene (1.5 mL) was added a solution of lithium bis(trimethylsilyl)amide 1M in THF (0.37 mL, 3.0 equiv) and piperidine (24 μL, 2.0 equiv). The reaction mixture was sparged with Argon for 10 minutes before addition of Pd-PEPPSI-iPent (9.7 mg, 0.1 equiv) The reaction mixture was subjected to microwave irradiation at 60° C. for 30 minutes. The reaction mixture was hydrolysed with water (20 mL) and extracted with EtOAc (20 mL). The organic layer was washed with brine, dried over magnesium sulfate then concentrated to dryness. The crude was purified by flash chromatography (Merck 60®, CyHex/EtOAc 80:20 to EtOAc 100%) then by preparative HPLC (column B, 5% to 100% ACN/H₂O), to obtain Example 82 (23 mg, 46%) as a yellow solid.

¹H-NMR (DMSO-d₆, 300 MHz) δ: 0.81 (d, J 6.7 Hz, 6H, CH—CH₃); 1.48-1.58 (m, 2H, N—CH₂—CH₂—CH₂); 1.64-1.75 (m, 10H, 2N—CH₂—CH₂—CH₂+2 C—CH₃); 2.06-2.19 (m, 1H, CH—CH₃); 2.91-2.98 (m, 4H, 2N—CH₂—CH₂—CH₂); 3.48-3.54 (m, 2H, N—CH₂); 3.97 (d, J 7.3 Hz, 2H, CH₂—CH); 7.11 (s, 1H, Ar); 7.24 (d, J 8.1 Hz, 1H, Ar); 8.04 (d, J 8.1 Hz, 1H, Ar); 8.06-8.10 (bs, 1H, NH), one N—CH₂ signal not observed.

M/Z (M+H)⁺: 412.4.

Mp: 233-237° C.

### Compound 201: methyl 3-(4-chloro-3-fluorophenyl)-1-isobutyl-1H-indazole-6-carboxylate

Under inert atmosphere, to a solution of methyl 1H-indazole-6-carboxylate (1.00 g) in DMF (35 mL) was added NIS (1.28 g, 1.0 equiv) and sodium hydride (60% in mineral oil) (68.1 mg, 0.3 equiv) at 0° C. The reaction was stirred at 25° C. for 1 hour, then 1-iodo-2-methylpropane (1.4 mL, 2.2 equiv) was added at 0° C. followed by sodium hydride (60% in mineral oil) (272 mg, 1.2 equiv). The reaction mixture was stirred at 0° C. for 1 hour then at 25° C. for 3 hours. The reaction mixture was diluted with aqueous sodium carbonate (10 mL, 2 molar, 3.5 equiv) at 0° C. At room temperature (4-chloro-3-fluorophenyl)boronic acid (1.09 g, 1.1 equiv) was added and the reaction mixture was sparged with Argon for 10 min before addition of [1,1′-Bis(diphenylphosphino)ferrocene]palladium(II) dichloride (208 mg, 0.05 equiv). The reaction mixture was heated at 80° C. for 18 hours then another portion of (4-chloro-3-fluorophenyl)boronic acid (396 mg, 0.4 equiv) and [1,1′-Bis(diphenylphosphino)ferrocene]palladium(II) dichloride (104 mg, 0.025 equiv) were added and heating was continued for another 6 hours. The reaction mixture was hydrolyzed with NH4Cl (sat. aq., 100 mL) diluted with water (200 mL) then extracted with EtOAc (3×80 mL). The organic layer was dried over magnesium sulfate then concentrated to dryness. The crude was purified by flash chromatography (Interchim® 50 μm, CyHex 100% to CyHex/EtOAc 40:60) to obtain Compound 201 (0.93 g, 45%) as a white solid.

M/Z (M[³⁵Cl]+H)⁺: 361.2.

### Compound 202: 3-(4-chloro-3-fluorophenyl)-1-isobutyl-1H-indazole-6-carboxylic acid

Compound 202 was obtained (900 mg, n.d.) as a white solid according to general procedure (IXa) starting from Compound 201 (900 mg).

M/Z (M[³⁵Cl]+H)⁺: 347.2

### Example 83: 4-(3-(4-chloro-3-fluorophenyl)-1-isobutyl-1H-indazole-6-carbonyl)-3,3-dimethylpiperazin-2-one

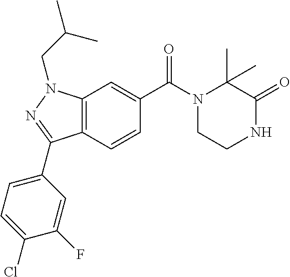 [SMILES: CC(C)Cn1nc(-c2ccc(Cl)c(F)c2)c2ccc(C(=O)N3CCNC(=O)C3(C)C)cc21]

Example 83 was prepared according to general procedure (Xa) starting from Compound 202 (80 mg) and 3,3-dimethylpiperazin-2-one (59 mg, 2 equiv), and using N,N-diisopropylethylamine (3.0 equiv). The crude was purified by flash chromatography (Merck 60®, CyHex 100% to EtOAc 100%) then by preparative HPLC (column B, 5% to 100% ACN/H₂O), to obtain Example 83 (46 mg, 44%) as a white solid.

¹H-NMR (DMSO-d₆, 300 MHz) δ: 0.89 (d, J 6.7 Hz, 6H, CH—CH₃); 1.74 (s, 6H, 2 C—CH₃); 2.19-2.33 (m, 1H, CH—CH₃); 3.19-3.28 (m, 2H, N—CH₂); 3.34-3.42 (m, 2H, N—CH₂); 4.35 (d, J 7.3 Hz, 2H, CH₂—CH); 7.26 (d, J 8.4 Hz, 1H, Ar); 7.67-7.76 (m, 1H, Ar); 7.82-7.96 (m, 3H, 2 Ar+NH); 8.09-8.19 (m, 2H, Ar) M/Z (M[³⁵Cl]+H)⁺: 457.3.

Mp: 202-206° C.

### Example 84: 4-(3-(2,4-dichlorophenyl)-1-isobutyl-1H-pyrrolo[2,3-b]pyridine-6-carbonyl)-3,3-dimethylpiperazin-2-one

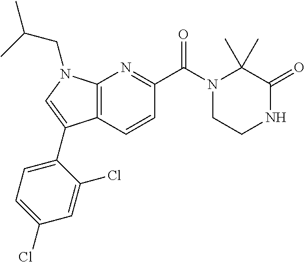 [SMILES: CC(C)Cn1cc(-c2ccc(Cl)cc2Cl)c2ccc(C(=O)N3CCNC(=O)C3(C)C)nc21]

Example 84 was prepared according to general procedure (XVIIb) starting from Compound 99 (100 mg) and 2,4-dichlorophenyl boronic acid (90 mg, 2.0 equiv). The crude residue was purified by flash chromatography (Merck 60®, CyHex 100% to EtOAc 100%) then recrystallized from iPr₂O/iPrOH (9:1) to obtain Example 84 (13 mg, 11%) as a white solid.

¹H-NMR (DMSO-d₆, 300 MHz) δ: 0.88 (d, J 6.6 Hz, 6H, 2 CH—CH₃); 1.72 (s, 6H, 2N—C—CH₃); 2.17-2.23 (m, 1H, CH—CH₃); 3.51-3.56 (m, 2H, N—CH₂); 4.15 (d, J 7.2 Hz, 2H, CH₂—CH); 7.41 (d, J 8.2 Hz, 1H, Ar); 7.51-7.54 (m, 2H, Ar); 7.61 (d, J 8.2 Hz, 1H, Ar); 7.77 (d, J 2.1 Hz, 1H, Ar); 8.01-8.04 (m, 2H, Ar); 8.12 (bs, 1H, NH). one N—CH₂ signal not observed.

M/Z (M[³⁵Cl₂]+H)⁺: 473.3.

Mp: 94-98° C.

### Compound 203: 1-(4-chlorophenyl)-3-isobutyl-1H-pyrazolo[4,3-b]pyridine

Hydrazone was prepared according to general procedure (XXVa) starting from Compound 108 (300 mg) and (4-chlorophenyl)hydrazine hydrochloride (353 mg, 1.5 equiv). The crude was directly cyclised according to general procedure (XXVII). The crude was purified by flash chromatography (Merck 60®, CyHex 100% to CyHex/EtOAc 80:20) to obtain Compound 203 (230 mg, 50%) as a yellow oil.

M/Z (M[³⁵Cl]+H)⁺: 286.2

### Compound 204: 1-(4-chlorophenyl)-3-isobutyl-1H-pyrazolo[4,3-b]pyridine 4-oxide

Compound 204 was prepared according to general procedure (XIXb) starting from Compound 203 (230 mg). The crude residue was purified by flash chromatography (Merck 60®, DCM 100% to DCM/MeOH 80:20) to obtain Compound 204 (130 mg, 54%) as a white solid.

M/Z (M[³⁵Cl]+H)⁺: 302.3

### Compound 205: 1-(4-chlorophenyl)-3-isobutyl-1H-pyrazolo[4,3-b]pyridine-5-carbonitrile

Compound 205 was prepared according to general procedure (XX) starting from Compound 204 (130 mg). The crude residue was purified by flash chromatography (Merck 60®, CyHex 100% to CyHex/EtOAc 80:20) to obtain Compound 205 (80 mg, 60%) as a clear oil.

M/Z (M[³⁵Cl]+H)⁺: 311.2

### Compound 206: 1-(4-chlorophenyl)-3-isobutyl-1H-pyrazolo[4,3-b]pyridine-5-carboxamide

Compound 206 was obtained (88 mg, n.d.) as a white solid according to general procedure (VII) starting from Compound 205 (80 mg).

M/Z (M[³⁵Cl]+H)⁺: 329.2

### Compound 207: methyl 1-(4-chlorophenyl)-3-isobutyl-1H-pyrazolo[4,3-b]pyridine-5-carboxylate

Compound 207 was prepared according to general procedure (VIII) starting from Compound 206 (88 mg). The crude was purified by flash chromatography (Merck 60®, CyHex 100% to EtOAc 100%) to obtain Compound 207 (75 mg, 85%) as a white solid.

M/Z (M[³⁵Cl]+H)⁺: 344.2

### Compound 208: 1-(4-chlorophenyl)-3-isobutyl-1H-pyrazolo[4,3-b]pyridine-5-carboxylic acid

Compound 208 was obtained (70 mg, 97%) as a white solid according to general procedure (IXa) starting from Compound 207 (75 mg).

M/Z (M[³⁵Cl]+H)⁺: 330.3

### Example 85: 4-(1-(4-chlorophenyl)-3-isobutyl-1H-pyrazolo[4,3-b]pyridine-5-carbonyl)-3,3-dimethylpiperazin-2-one

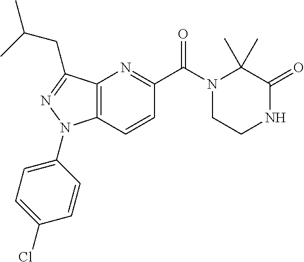 [SMILES: CC(C)Cc1nn(-c2ccc(Cl)cc2)c2ccc(C(=O)N3CCNC(=O)C3(C)C)nc12]

Example 85 was prepared according to general procedure (Xa) starting from Compound 208 (67 mg) and 3,3-dimethylpiperazin-2-one (31 mg, 1.2 equiv), and using N,N-diisopropylethylamine (3.0 equiv). The crude was purified by flash chromatography (Interchim® 50 μm, CyHex 100% to EtOAc 100%). The residue was triturated in ACN and the slurry was filtered to obtain Example 85 (52 mg, 58%) as a white solid.

¹H-NMR (DMSO-d₆, 400 MHz) δ: 0.98 (d, J 6.7 Hz, 6H, 2 CH—CH₃); 1.73 (s, 6H, 2 C—CH₃); 2.20-2.30 (m, 1H, CH—CH₃); 2.95 (d, J 7.1 Hz, 2H, CH₂—CH); 3.49-3.55 (m, 2H, N—CH₂); 7.65 (t, J 8.8 Hz, 2H, Ar); 7.73 (d, J 8.9 Hz, 1H, Ar); 7.85 (d, J 8.8 Hz, 1H, Ar); 8.10-8.13 (bs, 1H, NH), 8.41 (d, J 8.9 Hz, 1H, Ar), one N—CH₂ signal not observed.

M/Z (M[³⁵Cl]+H)⁺: 440.3.

Mp: 225-230° C.

### Compound 209: 3-(4-chloro-3-fluorophenyl)-3-oxopropanenitrile

Under inert atmosphere, to a solution of methyl 4-chloro-3-fluorobenzoate (500 mg, 1 equiv) in ACN (15 mL) was added sodium hydride (120 mg, 1.1 equiv) at 0° C. The reaction mixture was stirred for 15 min, then heated to reflux for 30 min. The reaction mixture was cooled to 50° C., then THF (10 mL) was added and the reaction mixture was heated to reflux overnight. The reaction mixture was cooled to room temperature, diluted with NH₄Cl (aq. sat. 30 mL) then extracted with DCM (3*20 mL). The organic layers were combined, dried with MgSO₄, filtered and concentrated under reduced pressure to obtain Compound 209 (340 mg, 62%) as an orange oil that solidifies upon standing.

¹H-NMR (DMSO-d₆, 400 MHz) δ: 4.05 (m, 2H, CH₂); 7.58 (d, J 8.8 Hz, 1H, Ar); 7.65 (dd, J 2.4, 9.0 Hz, Ar); 7.72 (dd, J 2.0, 9.0 Hz, 1H, Ar);

### Compound 210: 1-(tert-butyl)-3-(4-chloro-3-fluorophenyl)-1H-pyrazol-5-amine

To a solution of Compound 209 (993 mg, 1 equiv) in EtOH (17 mL) was added tert-butylhydrazine hydrochloride (626 mg, 1 equiv) and N-ethyl-N-isopropylpropan-2-amine (715 mg, 1.1 equiv). The reaction mixture was subjected to microwave irradiation at 150° C. for 1 hour. The reaction mixture was concentrated to dryness. The crude was purified by flash chromatography (CyHex 100% to DCM 100%) to obtain Compound 210 (1.14 g, 85%) as a yellow oil.

M/Z (M[³⁵Cl]+H)⁺: 268.2

### Compound 211: 4-bromo-1-(tert-butyl)-3-(4-chloro-3-fluorophenyl)-1H-pyrazol-5-amine

To a solution of Compound 210 (1.14 g, 1 equiv) in DCM (21 mL) was added N-bromosuccinimide (796 mg, 1.05 equiv). The reaction mixture was stirred at 0° C. for 30 min. The reaction mixture was hydrolysed with NaHCO₃ sat. (200 mL) then extracted with DCM (200 mL). The organic layer was washed with brine (200 mL), dried over magnesium sulfate then concentrated to dryness. The crude residue was purified by flash chromatography (CyHex 100% to CyHex/EtOAc 95:5) to obtain Compound 211 (1.32 g, 89%) as a purple solid.

M/Z (M[³⁵Cl⁸¹Br]+H)⁺: 348.0

### Compound 212: ethyl (E)-3-(5-amino-1-(tert-butyl)-3-(4-chloro-3-fluorophenyl)-1H-pyrazol-4-yl)acrylate

Under inert atmosphere, to a solution of Compound 211 (465 mg, 1 equiv) in triethylamine (13 mL) was added ethyl acrylate (1.34 g, 1.4 mL, 10 equiv). The reaction mixture was sparged with Argon for 10 min before addition of palladium tetrakis (155 mg, 0.10 equiv). The reaction mixture was heated at 120° C. for 2.5 hours then at 130° C. for 16 hours. The mixture was filtered through a pad of Celite washed with EtOAc (125 mL). The organic layer was washed with NH4Cl sat. (125 mL), brine (125 mL), dried over magnesium sulfate then concentrated to dryness. The crude was purified by flash chromatography (CyHex 100% to CyHex/EtOAc 80:20) to obtain Compound 212 (257 mg, 52%) as a beige solid.

M/Z (M[³⁵Cl]+H)⁺: 366.3

### Compound 213: 1-(tert-butyl)-3-(4-chloro-3-fluorophenyl)-1H-pyrazolo[3,4-b]pyridin-6-ol

Under inert atmosphere, to a solution of Compound 212 (257 mg, 1 equiv) in AcOH (7 mL) was added tributylphosphane (0.19 mL, 1.1 equiv). The reaction mixture was heated at 110° C. for 16 hours. The reaction mixture was hydrolyzed with NaHCO₃ sat. (100 mL) then extracted with EtOAc (100 mL). The organic layer was washed with brine (100 mL), dried over magnesium sulfate then concentrated to dryness. The crude was purified by flash chromatography (CyHex 100% to CyHex/EtOAc 80:20) to obtain Compound 213 (172 mg, 77%) as a beige solid.

M/Z (M[³⁵Cl]+H)⁺: 320.1

### Compound 214: 1-(tert-butyl)-3-(4-chloro-3-fluorophenyl)-6-fluoro-1H-pyrazolo[3,4-b]pyridine

Under inert atmosphere, to a solution of Compound 213 (127 mg, 1 equiv) in toluene (4 mL) was added PhenofluorMix (486 mg, 2 equiv). The reaction mixture was heated at 110° C. for 16 hours. The reaction mixture was diluted with EtOAc (30 mL), washed with water (30 mL), brine (30 mL), dried over magnesium sulfate then concentrated to dryness. The crude was purified by flash chromatography (CyHex 100% to CyHex/EtOAc 90:10) to obtain Compound 214 (94 mg, n.d.) as a yellow oil.

M/Z (M[³⁵Cl]+H)⁺: 322.1

### Compound 215: 1-(tert-butyl)-3-(4-chloro-3-fluorophenyl)-1H pyrazolo[3,4-b]pyridine-6-carbonitrile

To a solution of Compound 214 (94 mg, 1 equiv) in DMSO (2.9 mL) was added sodium cyanide (22 mg, 1.5 equiv). The reaction was subjected to microwave irradiation at 150° C. for 5 min. The reaction mixture was hydrolyzed with water (40 mL) then extracted with EtOAc (2*40 mL). The organic layer was washed with brine (40 mL), dried over magnesium sulfate then concentrated to dryness. The crude was purified by flash chromatography (CyHex 100% to CyHex/EtOAc 80:20) to obtain Compound 215 (78 mg, n.d.) as a brown oil.

M/Z (M[³⁵Cl]+H)⁺: 329.1

### Compound 216: 1-(tert-butyl)-3-(4-chloro-3-fluorophenyl)-1H-pyrazolo[3,4-b]pyridine-6-carboxamide

Compound 216 was obtained (72 mg, n.d.) as a yellow solid according to general procedure (VII) starting from Compound 215 (78 mg).

M/Z (M[³⁵Cl]+H)⁺: 347.1

### Compound 217: methyl 1-(tert-butyl)-3-(4-chloro-3-fluorophenyl)-1H-pyrazolo[3,4-b]pyridine-6-carboxylate

Compound 217 was prepared according to general procedure (VIII) starting from Compound 216 (72 mg). The crude was purified by flash chromatography (CyHex 100% to CyHex/EtOAc 80:20) to obtain Compound 217 (50 mg, n.d.) as a yellow oil.

M/Z (M[³⁵Cl]+H)⁺: 362.1

### Compound 218: 1-(tert-butyl)-3-(4-chloro-3-fluorophenyl)-1H-pyrazolo[3,4-b]pyridine-6-carboxylic acid

Compound 218 was obtained (51 mg, n.d.) as a yellow oil according to general procedure (IXa) starting from Compound 217 (50 mg).

M/Z (M[³⁵Cl]+H)⁺: 348.1

### Example 86: methyl 6-(4-(1-(tert-butyl)-3-(4-chloro-3-fluorophenyl)-1H-pyrazolo[3,4-b]pyridine-6-carbonyl)-3,3-dimethylpiperazin-1-yl)-2,4-dimethylnicotinate

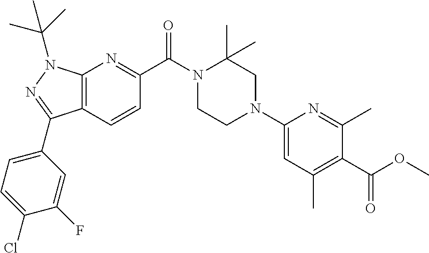 [SMILES: COC(=O)c1c(C)cc(N2CCN(C(=O)c3ccc4c(-c5ccc(Cl)c(F)c5)nn(C(C)(C)C)c4n3)C(C)(C)C2)nc1C]

Example 86 was prepared according to general procedure (Xa) starting from Compound 218 (48 mg) and Compound 95 (48 mg, 1.1 equiv), and using N,N-diisopropylethylamine (4.0 equiv). The crude was purified by flash chromatography (Interchim® 50 μm, CyHex 100% to CyHex/EtOAc 70:30) to obtain Example 86 (24 mg, 29%) as a yellow oil.

M/Z (M[³⁵Cl]+H)⁺: 607.4

### Example 87: 6-(4-(1-(tert-butyl)-3-(4-chloro-3-fluorophenyl)-1H-pyrazolo[3,4-b]pyridine-6-carbonyl)-3,3-dimethylpiperazin-1-yl)-2,4-dimethylnicotinic acid

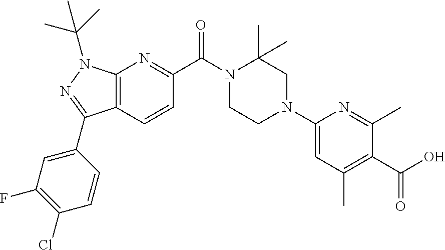 [SMILES: Cc1cc(N2CCN(C(=O)c3ccc4c(-c5ccc(Cl)c(F)c5)nn(C(C)(C)C)c4n3)C(C)(C)C2)nc(C)c1C(=O)O]

Example 87 was prepared according to general procedure (XI) starting from Example 86 (24 mg). The crude was purified by preparative HPLC (40% to 80% ACN/H₂O) to obtain Example 87 (16 mg, 68%) as a white solid.

¹H-NMR (DMSO-d₆, 400 MHz) δ: 1.56 (s, 6H, 2 CH₃); 1.85 (s, 9H, 3 CH₃); 2.28 (s, 3H, Pyr-CH₃); 2.39 (s, 3H, Pyr-CH₃); 3.53-3.56 (m, 2H, CH₂); 3.77-3.80 (m, 2H, CH₂); 3.94 (s, 2H, CH₂); 6.35 (bs, 1H, Ar); 7.47 (d, J 8.3 Hz, 1H, Ar); 7.74 (t, J 8.3 Hz, 1H, Ar); 7.90-7.93 (m, 1H, Ar); 7.98-8.01 (m, 1H, Ar); 8.75 (d, J 8.3 Hz, 1H, Ar).

M/Z (M[³⁵Cl]+H)⁺: 593.3

Mp: 140-147° C.

### Compound 219: (1,4-oxazepan-4-yl)(1H-pyrazolo[4,3-c]pyridin-6-yl)methanone

In a two chamber-reactor, a suspension of molybdenum hexacarbonyl (1.03 g, 0.5 equiv) in dioxane (17 mL) was prepared in the first chamber. A suspension of 6-bromo-1H-pyrazolo[4,3-c]pyridine (1.50 g), 1,4-oxazepane (1.50 g, 2 equiv), and triethylamine (2.1 mL, 2 equiv) in dioxane (25 mL) was prepared in the second chamber. The second chamber was sparged with Argon for 15 min, then XantPhos Pd G3 (0.14 g, 0.02 equiv) was added in chamber 2 and DBU (1.7 mL, 1.5 equiv) in chamber 1, and the two chamber reactor was heated at 85° C. for 18 hours. The mixture in chamber 2 was diluted in a MeOH/DCM mixture, basified with K₂CO₃ (3.15 g, 3 equiv) and concentrated over silica. The residue was purified by flash chromatography (Interchim® 50 μm, DCM 100% to DCM/MeOH 94:06) to obtain Compound 219 (1.22 g, 65%) as a yellow solid.

M/Z (M+H)⁺: 247.5

### Compound 220: (3-bromo-1H-pyrazolo[4,3-c]pyridin-6-yl)(1,4-oxazepan-4-yl)methanone

To a solution of Compound 219 (1.22 g) in DCM (25 mL) was added N-bromosuccinimide (0.92 g, 1.05 equiv). The reaction mixture was stirred at 25° C. for 2 hours. The reaction mixture was concentrated under reduced pressure and the residue was purified by flash chromatography (Interchim® 50 μm, DCM 100% to DCM/MeOH 90:10) to obtain Compound 220 (1.53 g, 95%) as a yellow solid.

M/Z (M[⁷⁹Br]+H)⁺: 325.4

### Compound 221: 2,2,2-trifluoroethyl 4-methylbenzenesulfonate

To a solution of 2-(2,2,2-trifluoroethoxy)ethan-1-ol (5 g) in DMF (100 mL) at 0° C. was added NaH 60% in mineral oil (2.60 g, 1.3 equiv). After 15 minutes, paratolylsulfonyl chloride (12.4 g, 1.3 equiv) was added and the mixture was let warm up at 25° C. for 1 hour. The mixture was hydrolysed in NaHCO₃ (sat. aq.) and extracted with EtOAc. The organic layers were combined, washed with brine, dried over MgSO₄, filtered, and concentrated under reduced pressure. The crude was purified by flash chromatography (Interchim® 50 μm, DCM 100% to DCM/MeOH 90:10) to obtain Compound 221 was obtained (12.8 g, n.d.) as a clear oil.

M/Z (M+H)⁺: 255.5

### Compound 222: (3-bromo-1-(2,2,2-trifluoroethyl)-1H-pyrazolo[4,3-c]pyridin-6-yl)(1,4-oxazepan-4-yl)methanone

Compound 222 was prepared according to general procedure (XIII) in DMA starting from Compound 220 (1.53 g) and Compound 221 (2.39 g, 2.0 equiv). The reaction mixture was subjected to microwave irradiation at 150° C. for 10 min. The crude was purified by flash chromatography (Interchim® 50 μm, DCM 100% to DCM/MeOH 95:05) to obtain Compound 222 (1.01 g, 49%) as a beige solid.

M/Z (M[⁷⁹Br]+H)⁺: 407.4

### Example 88: 4-(1-(4-chloro-3-fluorophenyl)-3-isobutyl-1H-pyrazolo[4,3-b]pyridine-5-carbonyl)piperazin-2-one

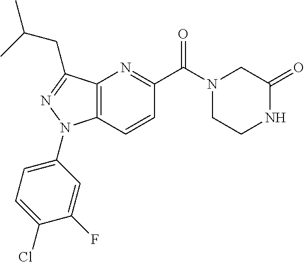 [SMILES: CC(C)Cc1nn(-c2ccc(Cl)c(F)c2)c2ccc(C(=O)N3CCNC(=O)C3)nc12]

Example 88 was prepared according to general procedure (Xa) starting from Compound 115 (60 mg) and piperazin-2-one (34 mg, 2 equiv), and using N,N-diisopropylethylamine (3.0 equiv). The crude was purified by preparative HPLC (column B, 5% to 100% ACN/H₂O), then taken up in DCM (0.5 mL) and precipitated in pentane (10 mL). The solid was recovered by filtration to obtain Example 88 (52 mg, 70%) as a white solid.

¹H-NMR (DMSO-d₆, 300 MHz) δ: 0.99 (d, J 6.6 Hz, 6H, 2 CH—CH₃), 2.21-2.32 (m, 1H, CH—CH₃), 2.96 (d, J 7.1 Hz, 2H, CH₂—CH), 3.32-3.36 (m, 2H, N—CH₂—CH₂—N), 3.79 (t, J 5.3 Hz, 1.2H, N—CH₂—CH₂—N one conformer), 3.88 (t, J 5.3 Hz, 0.8H, N—CH₂—CH₂—N, other conformer), 4.20 (s, 1.2H, N—CH₂—CO—NH, one conformer), 4.24 (s, 1.2H, N—CH₂—CO—NH, one conformer), 7.74 (dd, J 8.9, 2.3 Hz, 1H, Ar), 7.78-7.82 (m, 2H, Ar), 7.92 (dd, J 10.4, 2.3 Hz, 1H, Ar), 8.12 (bs, 0.4H, NH, other conformer), 8.14 (bs, 0.6H, NH, one conformer), 8.52 (d, J 8.9 Hz, 1H, Ar, other conformer), 8.53 (d, J 8.9 Hz, 1H, Ar, other conformer).

M/Z (M[³⁵Cl]+H)⁺: 430.2.

Mp: 185-190° C.

### Example 89: (3-(4-chloro-3-fluorophenyl)-1-(2,2,2-trifluoroethyl)-1H-pyrazolo[4,3-c]pyridin-6-yl)(1,4-oxazepan-4-yl)methanone

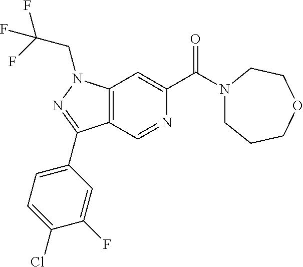 [SMILES: O=C(c1cc2c(cn1)c(-c1ccc(Cl)c(F)c1)nn2CC(F)(F)F)N1CCCOCC1]

Example 89 was prepared according to general procedure (XVIIb) starting from Compound 222 (75 mg) and (4-chloro-3-fluorophenyl)boronic acid (43 mg, 1.5 equiv). The crude residue was purified by flash chromatography (Interchim® 50 μm, DCM 100% to DCM/MeOH 95:05) then triturated in pentane. The recovered solid was freeze-dried to obtain Example 89 (73 mg, 71%) as a beige solid.

¹H-NMR (DMSO-d₆, 400 MHz) δ: 1.74-1.79 (m, 1H, CH—H); 1.91-1.97 (m, 1H, CH—H); 3.47-3.41 (m, 2H, CH₂); 3.63-3.66 (m, 1H, CH—H); 3.70-3.79 (m, 5H, CH—H+2 CH₂); 5.66 (q, J 9.0 Hz, 2H, CF₃—CH₂); 7.79 (t, J 8.1 Hz, 1H, Ar); 7.96-7.99 (m, 1H, Ar); 8.05-8.09 (m, 1H, Ar); 8.19 (m, 1H, Ar); 9.49-9.51 (m, 1H, Ar).

M/Z (M+H)⁺: 457.0.

Mp: 84-88° C.

### Compound 223: (8-oxa-3-azabicyclo[3.2.1]octan-3-yl)(1H-pyrazolo[4,3-c]pyridin-6-yl)methanone

In a two chamber-reactor, a suspension of molybdenum hexacarbonyl (340 mg, 0.5 equiv) in dioxane (8.4 mL) was prepared in the first chamber. A suspension of 6-bromo-1H-pyrazolo[4,3-c]pyridine (500 mg), 8-oxa-3-azabicyclo[3.2.1]octane hydrochloride (754 mg, 2 equiv), and triethylamine (1.05 mL, 2 equiv) in dioxane (8.4 mL) was prepared in the second chamber. The second chamber was sparged with Argon for 15 min, then XantPhos Pd G3 (47 mg, 0.02 equiv) was added in chamber 2 and DBU (0.56 mL, 1.5 equiv) in chamber 1, and the two chamber reactor was heated at 85° C. for 18 hours. The mixture in chamber 2 was diluted in a MeOH/DCM mixture, basified with K₂CO₃ (1.57 g, 4.5 equiv) and concentrated over silica. The residue was purified by flash chromatography (Interchim® 50 μm, DCM 100% to DCM/MeOH 94:06) to obtain Compound 223 (465 mg, 71%) as a yellow solid.

M/Z (M+H)⁺: 258.6

### Compound 224: (8-oxa-3-azabicyclo[3.2.1]octan-3-yl)(3-bromo-1H-pyrazolo[4,3-c]pyridin-6-yl)methanone

To a solution of Compound 223 (461 mg) in DCM (9 mL) was added N-bromosuccinimide (332 mg, 1.05 equiv). The reaction mixture was stirred at 25° C. for 2 hours. The reaction mixture was concentrated under reduced pressure and the residue was purified by flash chromatography (Interchim® 50 μm, DCM 100% to DCM/MeOH 90:10) to obtain Compound 224 (539 mg, 90%) as a white solid.

M/Z (M[⁷⁹Br]+H)⁺: 337.4

### Compound 225: (8-oxa-3-azabicyclo[3.2.1]octan-3-yl)(3-bromo-1-(2,2,2-trifluoroethyl)-1H-pyrazolo[4,3-c]pyridin-6-yl)methanone

Compound 225 was prepared according to general procedure (XIII) in DMA starting from Compound 224 (536 mg) and Compound 221 (808 mg, 2.0 equiv). The reaction mixture was subjected to microwave irradiation at 150° C. for 10 min. The crude was purified by flash chromatography (Interchim® 50 μm, DCM 100% to DCM/MeOH 95:05) to obtain Compound 225 (412 mg, 62%) as a beige solid.

M/Z (M[⁷⁹Br]+H)⁺: 419.5

### Example 90: (8-oxa-3-azabicyclo[3.2.1]octan-3-yl)(3-(4-chloro-3-fluorophenyl)-1-(2,2,2-trifluoroethyl)-1H-pyrazolo[4,3-c]pyridin-6-yl)methanone

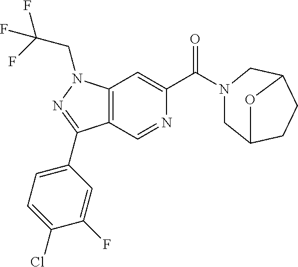 [SMILES: O=C(c1cc2c(cn1)c(-c1ccc(Cl)c(F)c1)nn2CC(F)(F)F)N1CC2CCC(C1)O2]

Example 90 was prepared according to general procedure (XVIIb) starting from Compound 225 (100 mg) and (4-chloro-3-fluorophenyl)boronic acid (62 mg, 1.5 equiv). The crude residue was purified by flash chromatography (Interchim® 50 μm, DCM 100% to DCM/MeOH 97:03) then triturated in pentane. The recovered solid was freeze-dried to obtain Example 90 (85 mg, 76%) as a beige solid.

¹H-NMR (DMSO-d₆, 400 MHz) δ: 1.73-1.91 (m, 4H, 2 CH₂); 3.05-3.08 (m, 1H, CH—H); 3.33-3.40 (m, 2H, CH₂); 4.21-4.24 (m, 2H, CH₂); 4.43-4.44 (m, 1H, CH—H); 5.62-5.70 (m, 2H, CH₂); 7.79 (t, J 8.1 Hz, 1H, Ar); 7.96-7.98 (m, 1H, Ar); 8.05-8.08 (m, 1H, Ar); 8.21 (s, 1H, Ar); 9.51 (m, 1H, Ar).

M/Z (M+H)⁺: 469.0.

Mp: 198-200° C.

### Example 91: 4-(1-isobutyl-3-(1-methyl-1,2,3,6-tetrahydropyridin-4-yl)-1H-pyrrolo[2,3-b]pyridine-6-carbonyl)-3,3-dimethylpiperazin-2-one hydrochloride

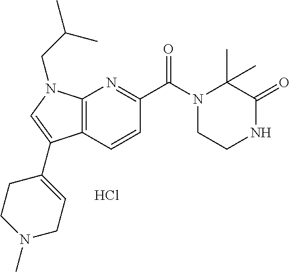 [SMILES: CC(C)Cn1cc(C2=CCN(C)CC2)c2ccc(C(=O)N3CCNC(=O)C3(C)C)nc21 ; Cl]

Example 91 was prepared according to general procedure (XVIIa) starting from Compound 99 (100 mg) and 1-methyl-1,2,3,6-tetrahydropyridine-4-boronic acid pinacol ester (110 mg, 2.0 equiv). The crude residue was purified by flash chromatography (Merck 60®, DCM 100% to DCM/MeOH 80:20) then the residue was taken up in DCM (5 mL), and HCl (4N in dioxane, 1 mL) was added. The solution was concentrated under reduced pressure and the residue taken up in ACN. The product was precipitated by addition in 10 mL Et₂O, and recovered by filtration to obtain Example 91 (29 mg, 26%) as a white solid.

¹H-NMR (DMSO-d₆, 300 MHz) δ: 0.84 (d, J 6.6 Hz, 6H, 2 CH₂—CH—CH₃), 1.71 (s, 6H, 2N—C—CH₃), 2.12-2.30 (m, 1H, N—CH₂—CH—CH₃), 2.73-2.88 (m, 5H), 3.45-3.70 (m, 3H), 3.70-4.05 (m, 2H), 4.06 (d, J 6.6 Hz, 2H, N—CH₂—CH—CH₃), 6.25 (bs, 1H, C═C—H), 7.42 (d, J 8.2 Hz, 1H, Ar), 7.92 (s, 1H, Ar), 8.13 (bs, 1H, NH), 8.39 (d, J 8.2 Hz, 1H, Ar), 10.34 (bs, 1H, HCl salt). 3 protons not observed.

M/Z (M+H)⁺: 424.2.

Compound 226: 3-isobutyl-1H-pyrazolo[4,3-b]pyridine 4-oxide Compound 226 was prepared according to general procedure (XIXb) starting from Compound 176 (2.20 g). The crude residue was purified by flash chromatography (CyHex 100% to CyHex/EtOAc 0:100 then DCM 100% to DCM/MeOH: 70/30) to obtain Compound 226 (1.27 g, 53%) as a yellow oil.

M/Z (M+H)⁺: 192.2

### Compound 227: 3-isobutyl-1H-pyrazolo[4,3-b]pyridine-5-carbonitrile

Compound 227 was prepared according to general procedure (XX) starting from Compound 226 (380 mg). The crude residue was purified by flash chromatography (DCM 100% to DCM/EtOAc 40:60) to obtain Compound 227 (332 mg, 83%) as a yellow solid.

M/Z (M+H)⁺: 201.2

### Compound 228: 3-isobutyl-1H-pyrazolo[4,3-b]pyridine-5-carboxamide

Compound 228 was obtained (1.16 g, n.d.), as a beige solid according to general procedure (VII) starting from Compound 227 (1.03 g).

M/Z (M+H)⁺: 219.2

### Compound 229: methyl 3-isobutyl-1H-pyrazolo[4,3-b]pyridine-5-carboxylate

Compound 229 was prepared according to general procedure (VIII) starting from Compound 228 (1.16 g). The residue was solubilized in EtOAc (100 mL), washed with water (100 mL), dried over magnesium sulfate and concentrated. The crude was purified by flash chromatography (CyHex 100% to CyHex/Et₂O 20:80) to obtain Compound 229 (570 mg, 46% over 2 steps) as a white solid.

M/Z (M+H)⁺: 234.2.

### Compound 230: methyl 3-isobutyl-1-(6-methoxypyridin-3-yl)-1H-pyrazolo[4,3-b]pyridine-5-carboxylate

Compound 230 was prepared according to general procedure (XXVIII) starting from Compound 229 (100 mg) and 2-Methoxy-5-pyridinylboronic acid. The crude was purified by flash chromatography (CyHex 100% to CyHex/EtOAc 50:50) to obtain Compound 230 (200 mg) as a yellow oil.

M/Z (M+H)⁺: 341.3.

### Compound 231: 3-isobutyl-1-(6-methoxypyridin-3-yl)-1H-pyrazolo[4,3-b]pyridine-5-carboxylic acid

Compound 231 was obtained (120 mg, 85% over 2 steps) as a white solid according to general procedure (IXa) starting from Compound 230 (0.43 mmol).

M/Z (M+H)⁺: 327.2

### Example 92: 4-(3-isobutyl-1-(6-methoxypyridin-3-yl)-1H-pyrazolo[4,3-b]pyridine-5-carbonyl)-3,3-dimethylpiperazin-2-one

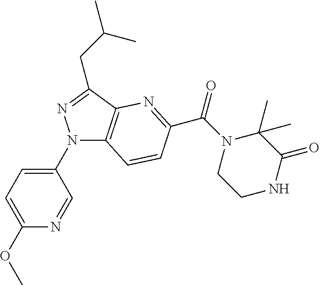 [SMILES: COc1ccc(-n2nc(CC(C)C)c3nc(C(=O)N4CCNC(=O)C4(C)C)ccc32)cn1]

Example 92 was prepared according to general procedure (Xa) starting from Compound 231 (60 mg) and 3,3-dimethylpiperazin-2-one (47 mg, 2.0 equiv), and using N,N-diisopropylethylamine (3.0 equiv). The crude was purified by flash chromatography (CyHex 100% to CyHex/EtOAc 0:100) to obtain Example 92 (52 mg, 65%) as a white solid.

¹H-NMR (CDCl₃, 300 MHz) δ: 1.04 (d, J 6.7 Hz, 6H, —CH—CH₂—(CH₃)₂); 1.93 (s, 6H, C(CH₃)₂); 2.27-2.36 (m, 1H, —CH—CH₂—(CH₃)₂); 3.02 (d, J 7.1 Hz, 2H, —CH—CH₂—(CH₃)₂); 3.63-3.67 (m, 2H, N—CH₂—CH₂—); 3.81-3.85 (m, 2H, N—CH₂—CH₂—); 4.03 (s, 3H, —OCH₃); 6.03 (bs, 1H, NH); 6.96 (d, J 8.8 Hz, 1H, Ar); 7.86 (d, J 8.8 Hz, 1H, Ar); 7.93 (dd, J 8.8, 2.7 Hz, 1H, Ar); 8.00 (d, J 8.8 Hz, 1H, Ar); 8.48 (d, J 2.7 Hz, 1H, Ar).

M/Z (M+H)⁺: 437.4

Mp: 189-192° C.

### Compound 232: methyl 3-isobutyl-1-(4-methoxyphenyl)-1H-pyrazolo[4,3-b]pyridine-5-carboxylate

Compound 232 was prepared according to general procedure (XXVIII) starting from Compound 229 (100 mg) and (4-methoxyphenyl)boronic acid. The crude was purified by flash chromatography (CyHex 100% to CyHex/EtOAc 50:50) to obtain Compound 232 (139 mg, 95%) as a white solid.

M/Z (M+H)⁺: 340.3.

### Compound 233: 3-isobutyl-1-(4-methoxyphenyl)-1H-pyrazolo[4,3-b]pyridine-5-carboxylic acid

Compound 233 was obtained (90 mg, 67%) as a yellow solid according to general procedure (IXa) starting from Compound 232 (139 mg).

M/Z (M+H)⁺: 326.2

### Example 93: 4-(3-isobutyl-1-(4-methoxyphenyl)-1H-pyrazolo[4,3-b]pyridine-5-carbonyl)-3,3-dimethylpiperazin-2-one

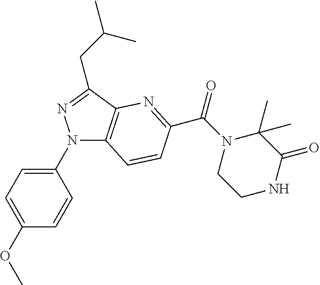 [SMILES: COc1ccc(-n2nc(CC(C)C)c3nc(C(=O)N4CCNC(=O)C4(C)C)ccc32)cc1]

Example 93 was prepared according to general procedure (Xa) starting from Compound 233 (78 mg) and 3,3-dimethylpiperazin-2-one (61 mg, 2.0 equiv), and using N,N-diisopropylethylamine (3.0 equiv). The crude was purified by flash chromatography (CyHex 100% to CyHex/EtOAc 0:100) to obtain Example 93 (68 mg, 65%) as a white solid.

¹H-NMR (CDCl₃, 300 MHz) δ: 1.04 (d, J 6.7 Hz, 6H, —CH—CH₂—(CH₃)₂); 1.93 (s, 6H, C(CH₃)₂); 2.28-2.37 (m, 1H, —CH—CH₂—(CH₃)₂); 3.02 (d, J 7.1 Hz, 2H, —CH—CH₂—(CH₃)₂); 3.64-3.68 (m, 2H, N—CH₂—CH₂—); 3.81-3.85 (m, 2H, N—CH₂—CH₂); 3.90 (s, 3H, —OCH₃); 6.05 (bs, 1H, NH); 7.08 (dd, J 6.8, 2.1 Hz, 2H, Ar); 7.59 (dd, J 6.8, 2.1 Hz, 2H, Ar); 7.83 (d, J 8.8 Hz, 1H, Ar); 8.01 (d, J 8.8 Hz, 1H, Ar).

M/Z (M+H)+436.4.

Mp: 182-185° C.

### Example 94: 8-(1-(4-chloro-3-fluorophenyl)-3-isobutyl-1H-pyrazolo[4,3-b]pyridine-5-carbonyl)-1-phenyl-1,3,8-triazaspiro[4.5]decan-4-one

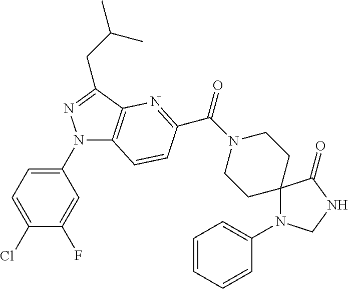 [SMILES: CC(C)Cc1nn(-c2ccc(Cl)c(F)c2)c2ccc(C(=O)N3CCC4(CC3)C(=O)NCN4c3ccccc3)nc12]

Example 94 was prepared according to general procedure (Xa) starting from Compound 115 (80 mg), 1-phenyl-1,3,8-triazaspiro[4.5]decan-4-one (110 mg, 2.0 equiv) and N,N-diisopropylethylamine (3.0 equiv). The crude residue was purified by flash chromatography (CyHex 100% to CyHex/EtOAc 0:100) then further purified by preparative HPLC (Column B, 5% to 100% ACN/H₂O), then freeze-dried with water to obtain Example 94 (113 mg, 88%) as a white solid.

¹H-NMR (DMSO-d₆, 300 MHz) δ: 0.76-0.84 (m, 6H, 2 CH₃); 1.63 (d, J 13.5 Hz, 1H, CHaHb-C—CHcHd); 1.80 (d, J 13.2 Hz, 1H, CHaHb-C—CHcHd); 2.13-2.23 (m, 1H, CH₂—CH—(CH₃)₂); 2.53-2.66 (m, 1H, CHaHb-C—CHcHd); 2.79-2.90 (m, 2H, CH₂—CH—(CH₃)₂); 2.90-3.05 (m, 1H, CHaHb-C—CHcHd); 3.46-3.58 (m, 1H, N—CHaHb); 3.75-3.77 (m, 2H, N—CH₂); 4.52-4.61 (m, 1H, N—CHaHb); 4.65 (s, 2H, N—CH₂—N); 6.78 (t, J 7.6 Hz, 1H, Ar); 6.89 (d, J 8.2 Hz, 2H, Ar); 7.25 (t, J 7.8 Hz, 2H, Ar); 7.70-7.83 (m, 3H, Ar); 7.92 (dd, J 10.5, 2.2 Hz, 1H, Ar); 8.53 (d, J 8.9 Hz, 1H, Ar); 8.86 (bs, 1H, NH).

M/Z (M[³⁵Cl]+H)⁺: 561.5.

Mp: 267-270° C.

### Compound 234: methyl 6-(4-(3-(4-chloro-3-fluorophenyl)-1-isobutyl-1H-pyrrolo[2,3-b]pyridine-6-carbonyl)-3,3-dimethylpiperazin-1-yl)-2,4-dimethylnicotinate

Compound 234 was prepared according to general procedure (Xb) starting from Compound 81 (49 mg), Compound 95 (53 mg) and N,N-diisopropylethylamine (4.0 equiv). The crude was purified by flash chromatography (CyHex 100% to CyHex/EtOAc 25:75) to obtain Compound 234 (80 mg) as a white solid.

M/Z (M[³⁵Cl]+H)⁺: 606.5

### Example 95: 6-(4-(3-(4-chloro-3-fluorophenyl)-1-isobutyl-1H-pyrrolo[2,3-b]pyridine-6-carbonyl)-3,3-dimethylpiperazin-1-yl)-2,4-dimethylnicotinic acid

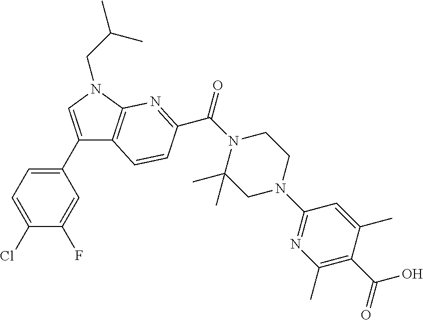 [SMILES: Cc1cc(N2CCN(C(=O)c3ccc4c(-c5ccc(Cl)c(F)c5)cn(CC(C)C)c4n3)C(C)(C)C2)nc(C)c1C(=O)O]

Example 95 was prepared according to general procedure (XIb) starting from Compound 234 (80 mg). The crude was purified by flash chromatography (DCM 100% to DCM/MeOH 80:20) and freeze-dried in MeOH/water to obtain Example 95 (36 mg, 78% over 2 steps) as a white solid.

¹H-NMR (DMSO-d₆, 300 MHz) δ: 0.92 (d, J 5.6, 6H, 2 CH₃); 1.53 (s, 6H, 2 CH₃); 2.25-2.36 (m, 7H, CH(CH₃)₂, 2 CH₃); 3.53-3.63 (m, 2H, CH₂); 3.78-3.85 (m, 2H, CH₂); 3.88-3.92 (m, 2H, CH₂); 4.11-4.17 (m, 2H, CH₂); 6.30 (s, 1H, Ar); 7.43 (d, J 8.4, 1H, Ar), 7.63-7.695 (m, 2H, Ar); 7.76-7.79 (m, 1H, Ar); 8.28 (s, 1H, Ar); 8.47 (d, J 8.4, 1H, Ar). 1 proton not observed.

M/Z(M[³⁵Cl]+H)⁺:592.4

### Compound 235: 6-chloro-3-(4-chloro-3-fluorophenyl)-1-isopropyl-1H-pyrrolo[2,3-b]pyridine

Compound 235 was prepared according to general procedure (IV) starting from Compound 3 (360 mg) and propan-2-amine (231 μL, 2.5 equiv) in DMA. The crude was purified by flash chromatography (Interchim® 50 μm, CyHex 100% to CyHex/Et₂O 90:10) to obtain Compound 235 (191 mg, 52%) as a white solid.

M/Z (M[³⁵Cl₂]+H)⁺: 323.2

### Compound 236: 3-(4-chloro-3-fluorophenyl)-1-isopropyl-1H-pyrrolo[2,3-b]pyridine-6-carbonitrile

Compound 236 was prepared according to general procedure (Va) starting from Compound 235 (190 mg). The crude was purified by flash chromatography (CyHex 100% to CyHex/Et₂O 80:20) to obtain Compound 236 (140 mg, 76%) as a white solid.

M/Z (M[³⁵Cl]+H)⁺: 314.1

### Compound 237: 3-(4-chloro-3-fluorophenyl)-1-isopropyl-1H-pyrrolo[2,3-b]pyridine-6-carboxamide

Compound 237 was prepared according to general procedure (VII) starting from Compound 236 (140 mg) and using potassium carbonate (2.0 equiv) and hydrogen peroxide aqueous 30% (3.0 equiv). The crude was purified by flash chromatography (CyHex 100% to CyHex/EtOAc 0:100) to obtain Compound 237 (128 mg, 87%) as a yellow solid.

M/Z (M[³⁵Cl]+H)⁺: 332.2.

### Compound 238: methyl 3-(4-chloro-3-fluorophenyl)-1-isopropyl-1H-pyrrolo[2,3-b]pyridine-6-carboxylate

Compound 238 was prepared according to general procedure (VIII) starting from Compound 237 (128 mg). The crude was purified by flash chromatography (CyHex 100% to CyHex/EtOAc 60:40) to obtain Compound 238 (108 mg, 80%) as a yellow solid.

M/Z (M[³⁵Cl]+H)⁺: 347.1

### Compound 239: 3-(4-chloro-3-fluorophenyl)-1-isopropyl-1H-pyrrolo[2,3-b]pyridine-6-carboxylic acid

Compound 239 was obtained (91 mg, 88%) as a yellow solid according to general procedure (IXa) starting from Compound 238 (108 mg).

M/Z (M[³⁵Cl]+H)⁺: 333.2

### Example 96: 4-(3-(4-chloro-3-fluorophenyl)-1-isopropyl-1H-pyrrolo[2,3-b]pyridine-6-carbonyl)-3,3-dimethylpiperazin-2-one

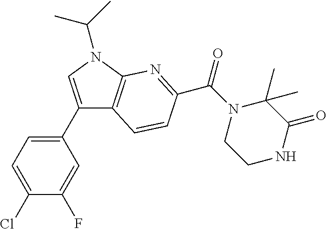 [SMILES: CC(C)n1cc(-c2ccc(Cl)c(F)c2)c2ccc(C(=O)N3CCNC(=O)C3(C)C)nc21]

Example 96 was prepared according to general procedure (Xa) starting from Compound 239 (40 mg) and 3,3-dimethylpiperazin-2-one (18 mg) and using N,N-diisopropylethylamine (3.0 equiv). The crude residue was purified by flash chromatography (CyHex 100% to CyHex/EtOAc 0:100), then triturated in Et2O to obtain Example 96 (25 mg, 47%) as a yellow solid.

¹H-NMR (DMSO-d₆+D₂O 300 MHz) δ: 1.50 (d, J 6.9 Hz, 6H, 2CH₃); 1.70 (s, 6H, 2CH₃); 3.29-3.33 (m, 2H, CH₂); 3.47-3.49 (m, 2H, CH₂); 5.03-5.12 (m, 1H, CH); 7.42 (d, J 8.1 Hz, 1H, Ar); 7.57-7.65 (m, 2H, Ar); 7.75 (d, J 10.8 Hz, 1H, Ar); 8.33 (s, 1H, Ar); 8.43 (d, J 8.1 Hz, 1H, Ar). 1 proton not observed.

M/Z(M[³⁵Cl]+H)⁺: 443.3.

Mp: 202-204° C.

### Compound 240: methyl 6-(4-(3-(4-chloro-3-fluorophenyl)-1-isopropyl-1H-pyrrolo[2,3-b]pyridine-6-carbonyl)-3,3-dimethylpiperazin-1-yl)-2,4-dimethylnicotinate

Compound 240 was prepared according to general procedure (Xa) starting from Compound 239 (42 mg) and Compound 95 (44 mg) and using N,N-diisopropylethylamine (4.0 equiv). The crude residue was purified by flash chromatography (CyHex 100% to CyHex/EtOAc 50:50) to obtain Compound 240 (64 mg, 86%) as a white solid.

M/Z (M[³⁵Cl]+H)⁺: 592.5.

### Example 97: 6-(4-(3-(4-chloro-3-fluorophenyl)-1-isopropyl-1H-pyrrolo[2,3-b]pyridine-6-carbonyl)-3,3-dimethylpiperazin-1-yl)-2,4-dimethyinicotinic acid

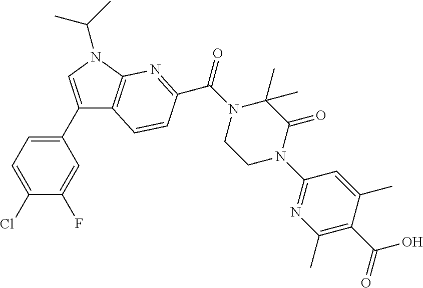 [SMILES: Cc1cc(N2CCN(C(=O)c3ccc4c(-c5ccc(Cl)c(F)c5)cn(C(C)C)c4n3)C(C)(C)C2=O)nc(C)c1C(=O)O]

Example 97 was prepared according to general procedure (XIb) starting from Compound 240 (64 mg). The crude was purified by flash chromatography (DCM 100% to DCM/MeOH 80:20) to obtain Example 97 (20 mg, 32%) as a yellow solid.

¹H-NMR (DMSO-d₆, 300 MHz) δ: 1.54 (s, 6H, (CH₃)₂); 1.54 (d, J 6.3 Hz, 6H, (CH₃)₂); 2.24 (s, 3H, CH₃); 2.36 (s, 3H, CH₃); 3.46-3.51 (m, 2H, CH₂); 3.75-3.80 (m, 2H, CH₂); 3.85-3.92 (m, 2H, CH₂); 5.11-5.16 (m, 1H, CH); 6.27 (s, 1H, Ar); 7.38 (d, J 8.2 Hz, 1H, Ar); 7.63-7.68 (m, 2H, Ar); 7.80 (d, J 8.2 Hz, 1H, Ar); 8.40 (s, 1H, Ar); 8.45 (d, J 8.2 Hz, 1H, Ar). 1 proton not observed.

M/Z (M[³⁵Cl]+H)⁺: 578.4.

### Compound 241: methyl 6-(4-(3-(4-chloro-3-fluorophenyl)-1-(tetrahydro-2H-pyran-4-yl)-1H-pyrrolo[2,3-b]pyridine-6-carbonyl)-3,3-dimethylpiperazin-1-yl)-2,4-dimethylnicotinate

Compound 241 was prepared according to general procedure (Xb) starting from Compound 82 (69 mg) and Compound 95 (69 mg) and using N,N-diisopropylethylamine (4.0 equiv). The crude residue was purified by flash chromatography (CyHex 100% to CyHex/EtOAc 0:100) to obtain Compound 241 (106 mg) as a white solid.

M/Z (M[³⁵Cl]+H)⁺: 634.5

### Example 98: 6-(4-(3-(4-chloro-3-fluorophenyl)-1-(tetrahydro-2H-pyran-4-yl)-1H-pyrrolo[2,3-b]pyridine-6-carbonyl)-3,3-dimethylpiperazin-1-1-2,4-dimethylnicotinic acid

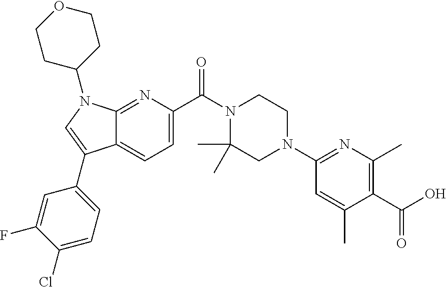 [SMILES: Cc1cc(N2CCN(C(=O)c3ccc4c(-c5ccc(Cl)c(F)c5)cn(C5CCOCC5)c4n3)C(C)(C)C2)nc(C)c1C(=O)O]

Example 98 was prepared according to general procedure (XIb) starting from Compound 241 (106 mg). The crude was purified by flash chromatography (DCM 100% to DCM/MeOH 90:10) and freeze-dried in EtOH/water to obtain Example 98 (60 mg, 63% over 2 steps) as a beige solid.

¹H-NMR (DMSO-d₆, 300 MHz) δ: 1.53 (s, 6H, 2 CH₃); 1.92-2.02 (m, 2H, CH₂); 2.21-2.25 (m, 5H, CH₂, CH₃); 2.37 (s, 3H, CH₃); 3.54-3.66 (m, 4H, 2 CH₂); 3.77-3.84 (m, 2H, CH₂); 3.88-3.93 (m, 2H, CH₂); 4.05-4.13 (m, 2H, CH₂); 4.93-5.05 (m, 1H, CH); 6.31 (s, 1H, Ar); 7.44 (d, J 8.1, 1H, Ar), 7.61-7.69 (m, 2H, Ar); 7.79-7.83 (m, 1H, Ar); 8.43-8.47 (m, 2H, Ar). 1 proton not observed.

M/Z(M[³⁵Cl]+H)⁺: 620.4

### Compound 242: tert-butyl 4-(5-(methoxycarbonyl)-4,6-dimethylpyridin-2-yl)piperazine-1-carboxylate

Compound 242 was prepared according to general procedure (XXI) starting from methyl 6-chloro-2,4-dimethylnicotinate (400 mg) and tert-butyl piperazine-1-carboxylate (411 mg). The crude residue was purified by flash chromatography (CyHex 100% to CyHex/EtOAc 75:25) to obtain Compound 242 (590 mg, 84%) as a white solid.

M/Z (M+H)⁺: 350.2

### Compound 243: methyl 2,4-dimethyl-6-(piperazin-1-yl)nicotinate dihydrochloride

Compound 243 was obtained (506 mg, 93%) as a white solid according to general procedure (XVIIIc) starting from Compound 242 (590 mg).

M/Z (M+H)⁺: 250.1

### Compound 244: methyl 6-(4-(1-(tert-butyl)-3-(4-chloro-3-fluorophenyl)-1H-pyrrolo[2,3-b]pyridine-6-carbonyl)piperazin-1-yl)-2,4-dimethylnicotinate

Compound 244 was prepared according to general procedure (Xa) starting from Compound 78 (60 mg) and Compound 243 (61 mg) and using N,N-diisopropylethylamine (5.0 equiv). The crude residue was purified by flash chromatography (CyHex 100% to CyHex/EtOAc 60:40) to obtain Compound 244 (90 mg, 90%) as a yellow solid. M/Z (M[³⁵Cl]+H)⁺: 578.4

### Example 99: 6-(4-(1-(tert-butyl)-3-(4-chloro-3-fluorophenyl)-1H-pyrrolo[2,3-b]pyridine-6-carbonyl)piperazin-1-yl)-2,4-dimethylnicotinic acid

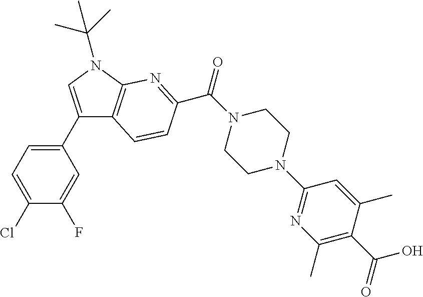 [SMILES: Cc1cc(N2CCN(C(=O)c3ccc4c(-c5ccc(Cl)c(F)c5)cn(C(C)(C)C)c4n3)CC2)nc(C)c1C(=O)O]

Example 99 was prepared according to general procedure (XIb) starting from Compound 244 (88 mg). The crude was purified by flash chromatography (DCM 100% to DCM/MeOH 80:20) to obtain Example 99 (36 mg, 42%) as a yellow solid.

¹H-NMR (DMSO-d₆, 300 MHz) δ: 1.81 (s, 9H, C(CH₃)₃); 2.23 (s, 3H, CH₃); 2.34 (s, 3H, CH₃), 3.55-3.63 (m, 2H, CH₂); 3.63-3.70 (m, 2H, CH₂); 3.70-3.75 (m, 2H, CH₂); 3.75-3.84 (m, 2H, CH₂); 6.54 (s, 1H, Ar), 7.49 (d, J 8.3 Hz, 1H, Ar); 7.65-7.70 (m, 2H, Ar); 7.83 (dd, J 11.1 Hz, 1.7 Hz, 1H, Ar); 8.23 (s, 1H, Ar); 8.45 (d, J 8.3 Hz, 1H, Ar). 1 proton not observed.

M/Z (M[³⁵Cl]+H)⁺: 564.5.

### Compound 245: methyl 6-((1-(tert-butoxycarbonyl)piperidin-4-yl)amino)-2,4-dimethylnicotinate

Compound 245 was prepared according to general procedure (XXI) starting from methyl 6-chloro-2,4-dimethylnicotinate (250 mg) and tert-butyl 4-aminopiperidine-1-carboxylate (276 mg). The crude residue was purified by flash chromatography (CyHex 100% to CyHex/EtOAc 50:50) to obtain Compound 245 (359 mg, 78%) as a white solid.

M/Z (M+H)⁺: 364.1

### Compound 246: methyl 2,4-dimethyl-6-(piperidin-4-ylamino)nicotinate hydrochloride

Compound 246 was obtained (250 mg, 84%) as a beige solid according to general procedure (XVIIIc) starting from Compound 245 (359 mg).

M/Z (M+H)⁺: 264.1

### Compound 247: methyl 6-((1-(1-(tert-butyl)-3-(4-chloro-3-fluorophenyl)-1H-pyrrolo[2,3-b]pyridine-6-carbonyl)piperidin-4-yl)amino)-2,4-dimethylnicotinate

Compound 247 was prepared according to general procedure (Xb) starting from Compound 78 (60 mg) and Compound 246 (52 mg) and using N,N-diisopropylethylamine (4.0 equiv). The crude residue was purified by flash chromatography (CyHex 100% to CyHex/EtOAc 0:100) to obtain Compound 247 (98 mg) as a white solid.

M/Z (M[³⁵Cl]+H)⁺: 592.5

### Example 100: 6-((1-(1-(tert-butyl)-3-(4-chloro-3-fluorophenyl)-1H-pyrrolo[2,3-b]pyridine-6-carbonyl)piperidin-4-yl)amino)-2,4-dimethylnicotinic acid

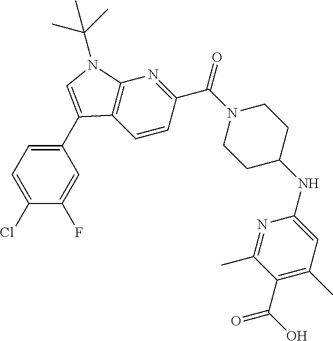 [SMILES: Cc1cc(NC2CCN(C(=O)c3ccc4c(-c5ccc(Cl)c(F)c5)cn(C(C)(C)C)c4n3)CC2)nc(C)c1C(=O)O]

Example 100 was prepared according to general procedure (XIb) starting from Compound 247 (98 mg). The crude was purified by flash chromatography (DCM 100% to DCM/MeOH 80:20) and freeze-dried in MeOH/water to obtain Example 100 (90 mg, 94% over 2 steps) as a white solid.

¹H-NMR (DMSO-d₆, 300 MHz) δ: 1.42-1.59 (m, 2H, 2 CH₂); 1.60 (s, 9H, C(CH₃)₃); 1.87-2.07 (m, 2H, CH₂); 2.18 (s, 3H, CH₃); 2.32 (s, 3H, CH₃); 3.06-3.30 (m, 2H, CH₂); 3.92-4.11 (m, 2H, CH₂); 4.37-4.47 (m, 1H, CH); 6.17 (s, 1H, Ar); 6.75 (d, J 7.8, 1H, nH); 7.42 (d, J 8.1, 1H, Ar); 7.59-7.67 (m, 2H, Ar); 7.81 (dd, J 11.7, 1.8 Hz, 1H, Ar); 8.20 (s, 1H, Ar); 8.43 (d, J 8.1 Hz, 1H, Ar), 12.46-12.60 (broad s, 1H, COOH).

M/Z(M[³⁵Cl]+H)⁺: 578.5

### Compound 248: tert-butyl 3-(5-(methoxycarbonyl)-4,6-dimethylpyridin-2-yl)-3,6-diazabicyclo[3.2.0]heptane-6-carboxylate

Compound 248 was prepared according to general procedure (XXI) starting from methyl 6-chloro-2,4-dimethylnicotinate (50 mg) and tert-butyl 3,6-diazabicyclo[3.2.0]heptane-6-carboxylate (50 mg). The crude residue was purified by flash chromatography (CyHex 100% to CyHex/EtOAc 75:25) to obtain Compound 248 (72 mg, 79%) as a white solid.

M/Z (M+H)⁺: 362.1

### Compound 249: methyl 6-(3,6-diazabicyclo[3.2.0]heptan-3-yl)-2,4-dimethylnicotinate hydrochloride

Compound 249 was obtained (70 mg) as a white solid according to general procedure (XVIIIc) starting from Compound 248 (72 mg).

M/Z (M+H)⁺: 262.1

### Compound 250: methyl 6-(6-(1-(tert-butyl)-3-(4-chloro-3-fluorophenyl)-1H-pyrrolo[2,3-b]pyridine-6-carbonyl)-3,6-diazabicyclo[3.2.0]heptan-3-yl)-2,4-dimethylnicotinate

Compound 250 was prepared according to general procedure (Xb) starting from Compound 78 (50 mg) and Compound 249 (59 mg) and using N,N-diisopropylethylamine (4.0 equiv). The crude residue was purified by flash chromatography (CyHex 100% to CyHex/EtOAc 0:100) to obtain Compound 250 (80 mg) as a beige solid. M/Z (M[³⁵Cl]+H)⁺: 590.3

### Example 101: 6-(6-(1-(tert-butyl)-3-(4-chloro-3-fluorophenyl)-1H-pyrrolo[2,3-b]pyridine-6-carbonyl)-3,6-diazabicyclo[3.2.0]heptan-3-yl)-2,4-dimethylnicotinic acid

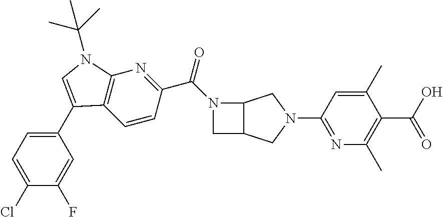 [SMILES: Cc1cc(N2CC3CN(C(=O)c4ccc5c(-c6ccc(Cl)c(F)c6)cn(C(C)(C)C)c5n4)C3C2)nc(C)c1C(=O)O]

Example 101 was prepared according to general procedure (XIb) starting from Compound 250 (94 mg). The crude was purified by flash chromatography (DCM 100% to DCM/MeOH 80:20) and freeze-dried in MeOH/water to obtain Example 101 (78 mg, 70% over 2 steps) as a yellow solid.

¹H-NMR (DMSO-d₆, 300 MHz) δ: 1.81 (s, 5.5H, one rotamer of C(CH₃)₃); 1.88 (s, 3.5H, other rotamer of C(CH₃)₃); 2.20 (s, 1.1H, other rotamer of CH₃); 2.24 (s, 1.9H, one rotamer of CH₃); 2.26 (s, 1.1H, other rotamer of CH₃); 2.36 (s, 1.9H, one rotamer of CH₃); 3.31-3.46 (m, 2H, CH₂); 3.75-3.80 (m, 0.4H, other rotamer of CH₂); 3.96-4.09 (m, 1.6H, one rotamer of CH₂); 4.21-4.42 (m, 2H, CH₂); 4.92-5.10 (m, 1.6H, one rotamer of CH and another rotamer of CH); 5.70-5.74 (m, 0.4H, another rotamer of CH); 6.22 (s, 0.4H, another rotamer of Ar); 6.33 (s, 0.6H, one rotamer of Ar); 7.56-7.67 (m, 2H, Ar); 7.74-7.81 (m, 1H, Ar); 7.86 (d, J 8.4, 0.4H, another rotamer of Ar); 7.90 (d, J 8.4, 0.6H, one rotamer of Ar); 8.19 (s, 0.6H, one rotamer of Ar); 8.23 (s, 0.4H, another rotamer of Ar); 8.40 (d, J 8.4, 0.6H, one rotamer of Ar); 8.44 (d, J 8.4, 0.4H, another rotamer of Ar). 1 proton not observed.

M/Z(M[³⁵Cl]+H)⁺: 576.4

### Compound 251: 2-(6-chloro-3-(4-chloro-3-fluorophenyl)-1H-pyrrolo[2,3-b]pyridin-1-yl)-2-methylpropan-1-ol

Compound 251 was prepared according to general procedure (IV) starting from Compound 3 (600 mg) and 2-amino-2-methylpropan-1-ol (90% in water, 466 mg, 2.5 equiv) in DMA. The crude was purified by flash chromatography (Interchim® 50 μm, CyHex 100% to CyHex/EtOAc 75:25), crystallized in Et₂O, to obtain Compound 251 (550 mg, 83%) as a white solid.

M/Z (M[³⁵Cl₂]+H)⁺: 353.1

### Compound 252: methyl 3-(4-chloro-3-fluorophenyl)-1-(1-hydroxy-2-methylpropan-2-yl)-1H-pyrrolo[2,3-b]pyridine-6-carboxylate

Compound 252 was prepared according to general procedure (XXIX) starting from Compound 251 (336 mg). The crude was purified by flash chromatography (Interchim® 20 μm, CyHex 100% to CyHex/EtOAc 60:40) to obtain Compound 252 (190 mg, 53%) as a yellow solid.

M/Z (M[³⁵Cl]+H)⁺: 377.7

### Compound 253: lithium 3-(4-chloro-3-fluorophenyl)-1-(1-hydroxy-2-methylpropan-2-yl)-1H-pyrrolo[2,3-b]pyridine-6-carboxylate

Compound 253 was obtained (200 mg) as a beige solid according to general procedure (IXb) starting from Compound 252 (155 mg).

M/Z (M[³⁵Cl]+H)⁺: 363.7

### Compound 254: methyl 6-(4-(3-(4-chloro-3-fluorophenyl)-1-(1-hydroxy-2-methylpropan-2-yl)-1H-pyrrolo[2,3-b]pyridine-6-carbonyl)-3,3-dimethylpiperazin-1-yl)-2,4-dimethylnicotinate

Compound 254 was prepared according to general procedure (Xc) starting from Compound 253 (152 mg), Compound 95 (142 mg) and N,N-diisopropylethylamine (4.0 equiv). The crude residue was purified by flash chromatography (Interchim® 50 μm, CyHex 100% to CyHex/EtOAc 70:30) to obtain Compound 254 (214 mg, 84% over 2 steps) as a yellow oil.

M/Z (M[³⁵Cl]+H)⁺: 622.3

### Example 102: 6-(4-(3-(4-chloro-3-fluorophenyl)-1-(1-hydroxy-2-methylpropan-2-yl)-1H-pyrrolo[2,3-b]pyridine-6-carbonyl)-3,3-dimethylpiperazin-1-yl)-2,4-dimethylnicotinic acid

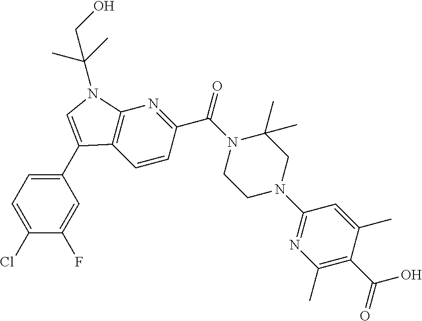 [SMILES: Cc1cc(N2CCN(C(=O)c3ccc4c(-c5ccc(Cl)c(F)c5)cn(C(C)(C)CO)c4n3)C(C)(C)C2)nc(C)c1C(=O)O]

Example 102 was prepared according to general procedure (XIb) starting from Compound 254 (94 mg). The crude was purified by preparative HPLC (30% to 70% ACN/H₂O), then freeze-dried with water to obtain Example 102 (38 mg, 56%) as a white solid.

¹H-NMR (DMSO-d₆, 300 MHz) δ: 1.54 (s, 6H, 2 CH₃); 1.75 (s, 6H, 2 CH₃); 2.27 (s, 3H, Ar—CH₃); 2.38 (s, 3H, Ar—CH₃); 3.52-3.56 (m, 2H, CH₂); 3.83-3.87 (m, 2H, CH₂); 3.92 (s, 2H, CH₂); 4.01-4.03 (m, 2H, HO—CH₂); 4.99-5.03 (m, 1H, OH); 6.33 (s, 1H, Ar); 7.40 (d, J 8.3 Hz, 1H, Ar); 7.59-7.67 (m, 2H, Ar); 7.79-7.83 (m, 1H, Ar); 8.17 (s, 1H, Ar); 8.43 (d, J 8.3 Hz, 1H, Ar). 1 proton not observed.

M/Z (M[³⁵Cl]+H)⁺: 608.2.

### Compound 255: 6-chloro-3-(4-chloro-3-fluorophenyl)-1-(pyrazin-2-ylmethyl)-1H-pyrrolo[2,3-b]pyridine

Compound 255 was prepared according to general procedure (IV) starting from Compound 3 (500 mg) and pyrazin-2-ylmethanamine (428 mg, 2.5 equiv) in DMA. The crude was purified by flash chromatography (Interchim® 50 μm, CyHex 100% to CyHex/EtOAc 50:50) to obtain Compound 255 (151 mg, 26%) as a white solid.

M/Z (M[³⁵Cl₂]+H)⁺: 373.1

### Compound 256: 3-(4-chloro-3-fluorophenyl)-1-(pyrazin-2-ylmethyl)-1H-pyrrolo[2,3-b]pyridine-6-carbonitrile

Compound 256 was prepared according to general procedure (Va) starting from Compound 255 (151 mg). The reaction was further subjected to microwave irradiation at 150° C. for 15 min. The crude was purified by flash chromatography (CyHex 100% to CyHex/EtOAc 50:50) to obtain Compound 256 (82 mg, 56%) as a yellow solid.

M/Z (M[³⁵Cl]+H)⁺: 364.7

### Compound 257:3-(4-chloro-3-fluorophenyl)-1-(pyrazin-2-ylmethyl)-1H-pyrrolo[2,3-b]pyridine-6-carboxamide

Compound 257 (62 mg, 72%) was prepared according to general procedure (VII) starting from Compound 256 (82 mg) and using potassium carbonate (2.0 equiv) and hydrogen peroxide aqueous 30% (3.0 equiv) to obtain a yellow solid.

M/Z (M[³⁵Cl]+H)⁺: 382.6.

### Compound 258: methyl 3-(4-chloro-3-fluorophenyl)-1-(pyrazin-2-ylmethyl)-1H-pyrrolo[2,3-b]pyridine-6-carboxylate

Compound 258 was obtained (57 mg, 88%) as a white solid according to general procedure (VIII) starting from Compound 257 (62 mg).

M/Z (M[³⁵Cl]+H)⁺: 397.7

### Compound 259: 3-(4-chloro-3-fluorophenyl)-1-(pyrazin-2-ylmethyl)-1H-pyrrolo[2,3-b]pyridine-6-carboxylic acid

To a solution of Compound 258 (57 mg, 1.0 equiv) in THF (1.4 mL) was added LiOH 1M in water (0.22 mL, 1.5 equiv). The reaction was stirred at 25° C. for 16 hour. The solvent was concentrated to dryness and the residue was solubilized in water (100 mL). The aqueous solution was washed with Et₂O (2×50 mL). 1M aqueous HCl was then added to until pH<3. The mixture was filtered, the solid was washed with water (2×10 mL) and dried to obtain Compound 259 (31 mg, 56%) as a white solid.

M/Z (M[³⁵Cl]+H)⁺: 383.7.

### Compound 260: methyl 6-(4-(3-(4-chloro-3-fluorophenyl)-1-(pyrazin-2-ylmethyl)-1H-pyrrolo[2,3-b]pyridine-6-carbonyl)-3,3-dimethylpiperazin-1-yl)-2,4-dimethylnicotinate

Compound 260 was prepared according to general procedure (Xa) starting from Compound 259 (30 mg) and Compound 95 (27 mg) and using N,N-diisopropylethylamine (4.0 equiv). The crude residue was purified by flash chromatography (CyHex 100% to CyHex/EtOAc 0:100) to obtain Compound 260 (50 mg, 99%) as a yellow oil.

M/Z (M[³⁵Cl]+H)⁺: 642.2.

### Example 103: 6-(4-(3-(4-chloro-3-fluorophenyl)-1-(pyrazin-2-ylmethyl)-1H-pyrrolo[2,3-b]pyridine-6-carbonyl)-3,3-dimethylpiperazin-1-yl)-2,4-dimethylnicotinic acid

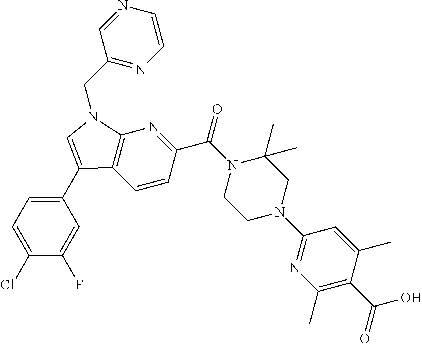 [SMILES: Cc1cc(N2CCN(C(=O)c3ccc4c(-c5ccc(Cl)c(F)c5)cn(Cc5cnccn5)c4n3)C(C)(C)C2)nc(C)c1C(=O)O]

Example 103 was prepared according to general procedure (XIb) starting from Compound 260 (50 mg). The crude was purified by preparative HPLC (30% to 70% ACN/H₂O), then freeze-dried with water to obtain Example 103 (1.0 mg, 2%) as a white solid.

¹H-NMR (DMSO-d₆, 300 MHz) δ: 1.48 (s, 6H, 2 CH₃); 2.29 (s, 3H, Ar—CH₃); 2.39 (s, 3H, Ar—CH₃); 3.26-3.28 (m, 2H, CH₂); 3.58-3.61 (m, 2H, CH₂); 3.86 (s, 2H, CH₂); 5.76 (s, 2H, N—CH₂); 6.26 (bs, 1H, Ar); 7.43 (d, J 8.2 Hz, 1H, Ar); 7.65-7.66 (m, 2H, Ar); 7.77-7.81 (m, 1H, Ar); 8.37 (bs, 1H, Ar); 8.50 (d, J 8.2 Hz, 1H, Ar); 8.55-8.59 (m, 2H, Ar); 8.72 (d, J 1.2 Hz, 1H, Ar); 12.68 (bs, 1H, COOH).

M/Z (M[³⁵Cl]+H)⁺: 628.2

### Compound 261: methyl 6-(4-(3-(4-chloro-3-fluorophenyl)-1-isobutyl-2-methyl-1H-pyrrolo[2,3-b]pyridine-6-carbonyl)-3,3-dimethylpiperazin-1-yl)-2,4-dimethylnicotinate

Compound 261 was prepared according to general procedure (Xc) starting from Compound 88 (30 mg), Compound 95 (55 mg) and N,N-diisopropylethylamine (4.0 equiv). The crude residue was purified by flash chromatography (CyHex 100% to CyHex/EtOAc 80:20) to obtain Compound 261 (47 mg, 91%) as a yellow oil.

M/Z (M[³⁵Cl]+H)⁺: 620.3

### Example 104: 6-(4-(3-(4-chloro-3-fluorophenyl)-1-isobutyl-2-methyl-1H-pyrrolo[2,3-b]pyridine-6-carbonyl)-3,3-dimethylpiperazin-1-yl)-2,4-dimethylnicotinic acid

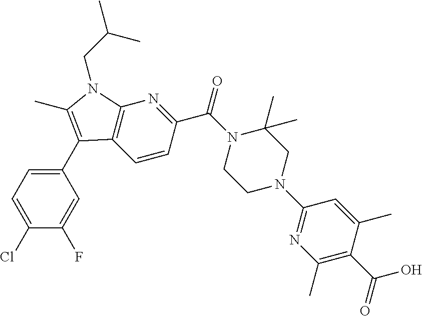 [SMILES: Cc1cc(N2CCN(C(=O)c3ccc4c(-c5ccc(Cl)c(F)c5)c(C)n(CC(C)C)c4n3)C(C)(C)C2)nc(C)c1C(=O)O]

Example 104 was prepared according to general procedure (XIb) starting from Compound 261 (47 mg). The crude was triturated in ACN (2*2 mL) then in Et₂O (2*2 mL) to obtain Example 104 (15 mg, 33%) as a white solid.

¹H-NMR (DMSO-d₆, 300 MHz) δ: 0.94 (d, J 6.6 Hz, 6H, 2 CH₃ of iBu); 1.53 (s, 6H, 2 CH₃); 2.19-2.24 (m, 1H, CH); 2.27 (s, 3H, Ar—CH₃); 2.39 (s, 3H, Ar—CH₃); 2.58 (s, 3H, Ar—CH₃); 3.59-3.63 (m, 2H, CH₂); 3.85-3.88 (m, 2H, CH₂); 3.91 (s, 2H, CH₂); 4.17 (d, J 7.6 Hz, 2H, CH₂); 6.36 (s, 1H, Ar); 7.36-7.39 (m, 2H, Ar); 7.52-7.56 (m, 1H, Ar); 7.69 (t, J 8.4 Hz, 1H, Ar); 8.04 (d, J 8.1 Hz, 1H, Ar). 1 proton not observed.

M/Z (M[³⁵Cl]+H)⁺: 606.3.

Mp: 127-140° C.

### Example 105: 4-(1-isobutyl-3-(6-methoxypyridin-3-yl)-1H-pyrrolo[2,3-b]pyridine-6-carbonyl)-3,3-dimethylpiperazin-2-one

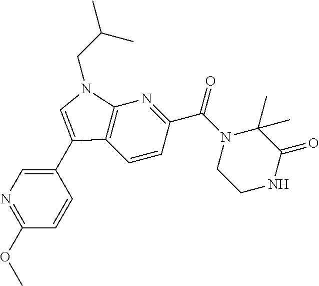 [SMILES: COc1ccc(-c2cn(CC(C)C)c3nc(C(=O)N4CCNC(=O)C4(C)C)ccc23)cn1]

Example 105 was prepared according to general procedure (XVIIa) starting from Compound 99 (100 mg) and 2-methoxy-5-(4,4,5,5-tetramethyl-1,3,2-dioxaborolan-2-yl)pyridine (69 mg, 1.2 equiv). The crude was purified by flash chromatography (Interchim® 20 μm, CyHex/EtOAc 50:50 to EtOAc 100%) and freeze-dried in ACN/water to obtain Example 105 (46 mg, 43%) as a white solid.

¹H-NMR (DMSO-d₆, 300 MHz) δ: 0.88 (d, J 6.6 Hz, 6H, CH(CH₃)₂); 1.72 (s, 6H, C(CH₃)₂); 2.15-2.25 (m, 1H, CH(CH₃)₂); 3.50-3.58 (m, 2H, NCH₂); 3.89 (s, 3H, OCH₃); 4.11 (d, J 7.2 Hz, 2H, CH₂CH), 6.93 (d, J 8.6 Hz, 1H, Ar), 7.42 (d, J 8.2 Hz, 1H, Ar), 8.05 (dd, J 8.6, 2.4 Hz, 1H, Ar), 8.10-8.15 (m, 2H, Ar+NH), 8.38 (d, J 8.2 Hz, 1H, Ar), 8.54 (d, J 2.4 Hz, 1H, Ar). 2 protons not observed.

M/Z (M+H)⁺: 436.3.

### Example 106: 4-(6-(2,2-dimethyl-3-oxopiperazine-1-carbonyl)-1-isobutyl-1H-pyrrolo[2,3-b]pyridin-3-yl)benzonitrile

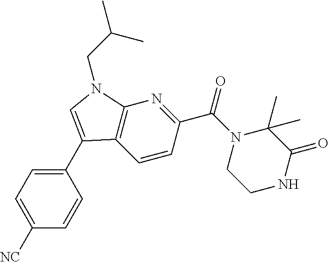 [SMILES: CC(C)Cn1cc(-c2ccc(C#N)cc2)c2ccc(C(=O)N3CCNC(=O)C3(C)C)nc21]

Example 106 was prepared according to general procedure (XVIIa) starting from Compound 99 (100 mg) and 4-(4,4,5,5-tetramethyl-1,3,2-dioxaborolan-2-yl)benzonitrile (68 mg, 1.2 equiv). The crude was purified by flash chromatography (Interchim® 20 μm, CyHex/EtOAc 50:50 to EtOAc 100%) and freeze-dried in ACN/water to obtain Example 106 (48 mg, 46%) as a white solid.

¹H-NMR (DMSO-d₆, 300 MHz) δ: 0.88 (d, J 6.5 Hz, 6H, CH(CH₃)₂); 1.72 (s, 6H, C(CH₃)₂); 2.16-2.26 (m, 1H, CH(CH₃)₂); 3.50-3.58 (m, 2H, NCH₂); 4.13 (d, J 7.2 Hz, 2H, CH₂CH), 7.49 (d, J 8.2 Hz, 1H, Ar), 7.88 (d, J 8.1 Hz, 2H, Ar), 7.96 (d, J 8.1 Hz, 2H, Ar), 8.12 (s, 1H, NH), 8.40 (s, 1H, Ar), 8.51 (d, J 8.2 Hz, 1H, Ar). 2 protons not observed.

M/Z (M+H)⁺: 430.3

### Example 107: 4-(3-(3,4-dichlorophenyl)-1-isobutyl-1H-pyrrolo[2,3-b]pyridine-6-carbonyl)-3,3-dimethylpiperazin-2-one

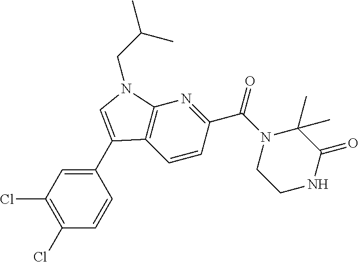 [SMILES: CC(C)Cn1cc(-c2ccc(Cl)c(Cl)c2)c2ccc(C(=O)N3CCNC(=O)C3(C)C)nc21]

Example 107 was prepared according to general procedure (XVIIa) starting from Compound 99 (100 mg) and (3,4-dichlorophenyl)boronic acid (56 mg, 1.2 equiv). The crude was purified by flash chromatography (Interchim® 20 μm, CyHex/EtOAc 50:50 to EtOAc 100%) then recrystallized from hot ACN (5 mL) to obtain Example 107 (37 mg, 32%) as a white solid.

¹H-NMR (DMSO-d₆, 300 MHz) δ: 0.88 (d, J 6.7 Hz, 6H, CH(CH₃)₂); 1.72 (s, 6H, C(CH₃)₂); 2.15-2.27 (m, 1H, CH(CH₃)₂); 3.50-3.58 (m, 2H, NCH₂); 4.11 (d, J 7.2 Hz, 2H, CH₂CH); 7.47 (d, J 8.2 Hz, 1H, Ar); 7.68 (d, J 8.4 Hz, 1H, Ar); 7.76 (dd, J 8.4, 2.1 Hz, 1H, Ar); 7.97 (d, J 2.1 Hz, 1H, Ar); 8.12 (s, 1H, NH); 8.32 (s, 1H, Ar); 8.44 (d, J 8.2 Hz, 1H, Ar). 2 protons not observed.

M/Z (M[³⁵Cl₂]+H)⁺: 473.3.

Mp: 215-220° C.

### Example 108: 4-(3-(3,5-difluorophenyl)-1-isobutyl-1H-pyrrolo[2,3-b]pyridine-6-carbonyl)-3,3-dimethylpiperazin-2-one

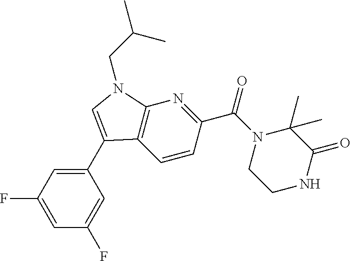 [SMILES: CC(C)Cn1cc(-c2cc(F)cc(F)c2)c2ccc(C(=O)N3CCNC(=O)C3(C)C)nc21]

Example 108 was prepared according to general procedure (XVIIa) starting from Compound 99 (100 mg) and 2-(3,5-difluorophenyl)-4,4,5,5-tetramethyl-1,3,2-dioxaborolane (71 mg, 1.2 equiv). The crude was purified by flash chromatography (Interchim® 20 μm, CyHex/EtOAc 50:50 to EtOAc 100%) then recrystallized from hot ACN (5 mL) to obtain Example 108 (31 mg, 28%) as a white solid.

¹H-NMR (DMSO-d₆, 300 MHz) δ: 0.88 (d, J 6.6 Hz, 6H, CH(CH₃)₂); 1.72 (s, 6H, C(CH₃)₂); 2.15-2.27 (m, 1H, CH(CH₃)₂); 3.50-3.58 (m, 2H, NCH₂); 4.11 (d, J 7.2 Hz, 2H, CH₂CH), 7.11 (tt, J 9.3 2.1 Hz, 1H, Ar), 7.43-7.56 (m, 3H, Ar), 8.12 (s, 1H, NH), 8.34 (s, 1H, Ar), 8.51 (d, J 8.2 Hz, 1H, Ar). 2 protons not observed.

M/Z (M+H)⁺: 441.3.

Mp: 165-170° C.

### Example 109: 4-(3-(6-(dimethylamino)pyridin-3-yl)-1-isobutyl-1H-pyrrolo[2,3-b]pyridine-6-carbonyl)-3,3-dimethylpiperazin-2-one

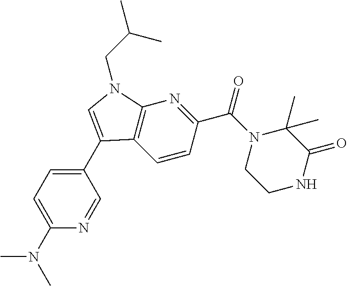 [SMILES: CC(C)Cn1cc(-c2ccc(N(C)C)nc2)c2ccc(C(=O)N3CCNC(=O)C3(C)C)nc21]

Example 109 was prepared according to general procedure (XVIIa) starting from Compound 99 (100 mg) and (6-(dimethylamino)pyridin-3-yl)boronic acid dihydrochloride (70 mg, 1.2 equiv) and potassium carbonate 1M in water (1.23 mL, 5.0 equiv). The crude was purified by flash chromatography (Interchim®20 μm, DCM 100% to DCM/MeOH 88:12) then recrystallized from hot ACN (5 mL) to obtain Example 109 (24 mg, 22%) as a white solid.

¹H-NMR (DMSO-d₆, 300 MHz) δ: 0.88 (d, J 6.6 Hz, 6H, CH(CH₃)₂); 1.72 (s, 6H, C(CH₃)₂); 2.15-2.25 (m, 1H, CH(CH₃)₂); 3.06 (s, 6H, N(CH₃)₂); 3.50-3.58 (m, 2H, NCH₂); 4.11 (d, J 7.2 Hz, 2H, CH₂CH); 6.75 (d, J 8.9 Hz, 1H, Ar); 7.39 (d, J 8.2 Hz, 1H, Ar); 7.84 (dd, J 8.9 2.4 Hz, 1H, Ar); 7.97 (s, 1H, Ar); 8.08-8.13 (m, 1H, NH), 8.32 (d, J 8.2 Hz, 1H, Ar), 8.47 (d, J 2.4 Hz, 1H, Ar). 2 protons not observed.

M/Z (M+H)⁺: 449.4

Mp: 205-210° C.

### Example 110: 5-(6-(2,2-dimethyl-3-oxopiperazine-1-carbonyl)-1-isobutyl-1H-pyrrolo[2,3-b]pyridin-3-yl)picolinonitrile

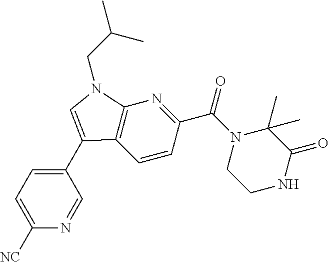 [SMILES: CC(C)Cn1cc(-c2ccc(C#N)nc2)c2ccc(C(=O)N3CCNC(=O)C3(C)C)nc21]

Example 110 was prepared according to general procedure (XVIIa) starting from Compound 99 (100 mg) and 5-(4,4,5,5-tetramethyl-1,3,2-dioxaborolan-2-yl)picolinonitrile (68 mg, 1.2 equiv). The crude was purified by flash chromatography (Interchim® 20 μm, CyHex/EtOAc 50:50 to EtOAc 100%) then recrystallized from hot ACN (5 mL) to obtain Example 110 (8.7 mg, 8%) as a white solid.

¹H-NMR (DMSO-d₆, 300 MHz) δ: 0.88 (d, J 6.6 Hz, 6H, CH(CH₃)₂); 1.72 (s, 6H, C(CH₃)₂); 2.15-2.27 (m, 1H, CH(CH₃)₂); 3.50-3.58 (m, 2H, NCH₂); 4.11 (d, J 7.2 Hz, 2H, CH₂CH); 7.50 (d, J 8.3 Hz, 1H, Ar); 8.07 (dd, J 8.2 0.5 Hz, 1H, Ar); 8.10-8.15 (m, 1H, NH); 8.40 (dd, J 8.2 2.3 Hz, 1H, Ar); 8.54 (s, 1H, Ar); 8.57 (d, J 8.3 Hz, 1H, Ar); 9.19 (dd, J 2.4 0.5 Hz, 1H, Ar). 2 protons not observed.

M/Z (M+H)⁺: 431.3.

Mp: 260-265° C.

### Example 111: 4-(1-isobutyl-3-(4-methoxyphenyl)-1H-pyrrolo[2,3-b]pyridine-6-carbonyl)-3,3-dimethylpiperazin-2-one

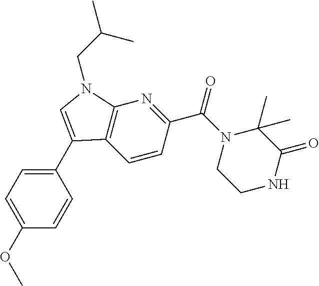 [SMILES: COc1ccc(-c2cn(CC(C)C)c3nc(C(=O)N4CCNC(=O)C4(C)C)ccc23)cc1]

Example 111 was prepared according to general procedure (XVIIa) starting from Compound 99 (100 mg) and 2-(4-methoxyphenyl)-4,4,5,5-tetramethyl-1,3,2-dioxaborolane (69 mg, 1.2 equiv). The crude was purified by flash chromatography (Interchim® 20 μm, CyHex/EtOAc 50:50 to EtOAc 100%) then recrystallized from hot ACN (5 mL) to obtain Example 111 (18 mg, 17%) as a white solid.

¹H-NMR (DMSO-d₆, 300 MHz) δ: 0.88 (d, J 6.6 Hz, 6H, CH(CH₃)₂); 1.72 (s, 6H, C(CH₃)₂); 2.15-2.25 (m, 1H, CH(CH₃)₂); 3.50-3.58 (m, 2H, NCH₂); 3.80 (s, 3H, OCH₃); 4.11 (d, J 7.2 Hz, 2H, CH₂CH); 7.02 (d, J 8.8 Hz, 2H, Ar); 7.42 (d, J 8.2 Hz, 1H, Ar); 7.64 (d, J 8.8 Hz, 2H, Ar); 8.00 (s, 1H, Ar); 8.08-8.16 (m, 1H, NH); 8.35 (d, J 8.2 Hz, 1H, Ar). 2 protons not observed.

M/Z (M+H)⁺: 435.3.

Mp: 195-200° C.

### Example 112: 4-(1-isobutyl-3-(2-(trifluoromethoxy)phenyl)-1H-pyrrolo[2,3-b]pyridine-6-carbonyl)-3,3-dimethylpiperazin-2-one

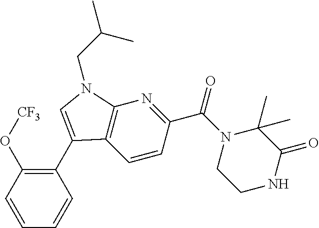 [SMILES: CC(C)Cn1cc(-c2ccccc2OC(F)(F)F)c2ccc(C(=O)N3CCNC(=O)C3(C)C)nc21]

Example 112 was prepared according to general procedure (XVIIa) starting from Compound 99 (100 mg) and (2-(trifluoromethoxy)phenyl)boronic acid (100 mg, 2.0 equiv). The crude was purified by flash chromatography (Interchim® 20 μm, CyHex 100% to CyHex/EtOAc 80:20) then recrystallized from hot ACN (5 mL) to obtain Example 112 (22 mg, 18%) as a white solid.

¹H-NMR (DMSO-d₆, 400 MHz) δ: 0.87 (d, J 6.7 Hz, 6H, CH(CH₃)₂); 1.72 (s, 6H, C(CH₃)₂); 2.17-2.25 (m, 1H, CH(CH₃)₂); 3.33-3.38 (m, 2H, NCH₂); 3.52-3.60 (m, 2H, NCH₂); 4.16 (d, J 7.3 Hz, 2H, CH₂CH), 7.44 (d, J 8.2 Hz, 1H, Ar), 7.46-7.54 (m, 3H, Ar), 7.71-7.78 (m, 1H, Ar), 7.98 (s, 1H, Ar), 8.07-8.10 (m, 1H, NH), 8.14 (d, J 8.2 Hz, 1H, Ar).

M/Z (M+H)⁺: 489.2

### Example 113: 4-(3-(2-fluorophenyl)-1-isobutyl-1H-pyrrolo[2,3-b]pyridine-6-carbonyl)-3,3-dimethylpiperazin-2-one

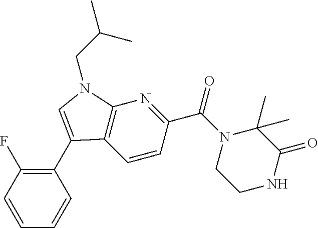 [SMILES: CC(C)Cn1cc(-c2ccccc2F)c2ccc(C(=O)N3CCNC(=O)C3(C)C)nc21]

Example 113 was prepared according to general procedure (XVIIa) starting from Compound 99 (100 mg) and (2-fluorophenyl)boronic acid (70 mg, 2.0 equiv). The crude was purified by flash chromatography (Interchim® 20 μm, CyHex 100% to CyHex/EtOAc 80:20) then recrystallized from hot ACN (5 mL) to obtain Example 113 (23 mg, 21%) as a white solid.

¹H-NMR (DMSO-d₆, 400 MHz) δ: 0.88 (d, J 6.7 Hz, 6H, CH(CH₃)₂); 1.72 (s, 6H, C(CH₃)₂); 2.16-2.29 (m, 1H, CH(CH₃)₂); 3.33-3.37 (m, 2H, NCH₂); 3.52-3.59 (m, 2H, NCH₂); 4.16 (d, J 7.3 Hz, 2H, CH₂CH), 7.28-7.40 (m, 3H, Ar), 7.44 (d, J 8.2 Hz, 1H, Ar), 7.74 (td, J 7.8, 1.4 Hz, 1H, Ar), 8.04 (d, J 1.4 Hz, 1H, Ar), 8.09 (bs, 1H, NH), 8.22 (dd, J 8.2, 1.8 Hz, 1H, Ar).

M/Z (M+H)⁺: 423.4

### Example 114: 4-(3-(3-fluorophenyl)-1-isobutyl-1H-pyrrolo[2,3-b]pyridine-6-carbonyl)-3,3-dimethylpiperazin-2-one

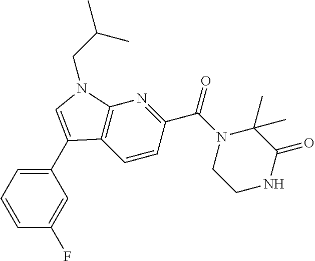 [SMILES: CC(C)Cn1cc(-c2cccc(F)c2)c2ccc(C(=O)N3CCNC(=O)C3(C)C)nc21]

Example 114 was prepared according to general procedure (XVIIa) starting from Compound 99 (100 mg) and (3-fluorophenyl)boronic acid (70 mg, 2.0 equiv). The crude was purified by flash chromatography (Interchim® 20 μm, CyHex 100% to CyHex/EtOAc 80:20) then recrystallized from hot ACN (5 mL) to obtain Example 114 (29 mg, 27%) as a white solid.

¹H-NMR (DMSO-d₆, 400 MHz) δ: 0.88 (d, J 6.7 Hz, 6H, CH(CH₃)₂); 1.72 (s, 6H, C(CH₃)₂); 2.18-2.29 (m, 1H, CH(CH₃)₂); 3.51-3.60 (m, 2H, NCH₂); 4.12 (d, J 7.2 Hz, 2H, CH₂CH), 7.09 (tdd, J 8.2, 2.4, 0.8 Hz, 1H, Ar), 7.45 (d, J 8.2 Hz, 1H, Ar), 7.49 (ddd, J 8.2, 7.7, 1.5 Hz, 1H, Ar), 7.54 (ddd, J 11.1, 2.4, 1.5 Hz, 1H, Ar), 7.60 (ddd, J 7.7, 1.2, 0.8 Hz, 1H, Ar), 8.09 (bs, 1H, NH), 8.23 (s, 1H, Ar), 8.45 (d, J 8.2 Hz, 1H, Ar). 2 protons not observed.

M/Z (M+H)⁺: 423.3

### Example 115: 4-(3-(4-chloro-2-fluorophenyl)-1-isobutyl-1H-pyrrolo[2,3-b]pyridine-6-carbonyl)-3,3-dimethylpiperazin-2-one

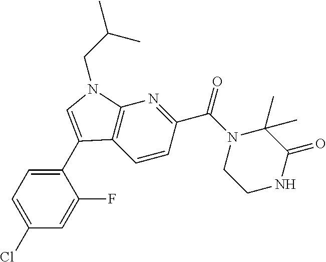 [SMILES: CC(C)Cn1cc(-c2ccc(Cl)cc2F)c2ccc(C(=O)N3CCNC(=O)C3(C)C)nc21]

Example 115 was prepared according to general procedure (XVIIa) starting from Compound 99 (100 mg) and (4-chloro-2-fluorophenyl)boronic acid (85 mg, 2 equiv). The crude was purified by flash chromatography (Interchim® 20 μm, CyHex 100% to CyHex/EtOAc 80:20) then recrystallized from hot ACN (5 mL) to obtain Example 115 (5.3 mg, 5%) as a white solid.

¹H-NMR (DMSO-d₆, 400 MHz) δ: 0.88 (d, J 6.7 Hz, 6H, CH(CH₃)₂); 1.72 (s, 6H, C(CH₃)₂); 2.16-2.26 (m, 1H, CH(CH₃)₂); 3.32-3.37 (m, 2H, NCH₂); 3.51-3.58 (m, 2H, NCH₂); 4.15 (d, J 7.2 Hz, 2H, CH₂CH), 7.39 (dd, J 8.4, 2.2 Hz, 1H, Ar), 7.45 (d, J 8.2 Hz, 1H, Ar), 7.57 (dd, J 10.7, 2.2 Hz, 1H, Ar), 7.77 (t, J 8.4 Hz, 1H, Ar), 8.07 (d, J 1.6 Hz, 1H, Ar), 8.09 (bs, 1H, NH), 8.22 (dd, J 8.2, 1.6 Hz, 1H, Ar).

M/Z (M[³⁵Cl]+H)⁺: 457.2

### Example 116: 4-(1-isobutyl-3-(4-(trifluoromethoxy)phenyl)-1H-pyrrolo[2,3-b]pyridine-6-carbonyl)-3,3-dimethylpiperazin-2-one

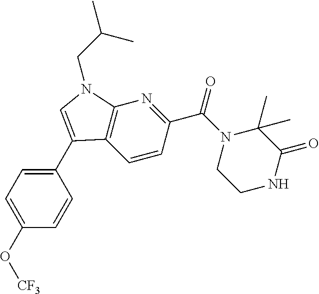 [SMILES: CC(C)Cn1cc(-c2ccc(OC(F)(F)F)cc2)c2ccc(C(=O)N3CCNC(=O)C3(C)C)nc21]

Example 116 was prepared according to general procedure (XVIIa) starting from Compound 99 (100 mg) and (4-(trifluoromethoxy)phenyl)boronic acid (100 mg, 2 equiv). The crude was purified by flash chromatography (Interchim® 20 μm, CyHex 100% to CyHex/EtOAc 80:20) then recrystallized from hot ACN (5 mL) to obtain Example 116 (19 mg, 15%) as a white solid.

¹H-NMR (DMSO-d₆, 400 MHz) δ: 0.88 (d, J 6.6 Hz, 6H, CH(CH₃)₂); 1.72 (s, 6H, C(CH₃)₂); 2.18-2.28 (m, 1H, CH(CH₃)₂); 3.33-3.37 (m, 2H, NCH₂); 3.52-3.61 (m, 2H, NCH₂); 4.13 (d, J 7.2 Hz, 2H, CH₂CH), 7.42-7.48 (m, 3H, Ar), 7.81-7.88 (m, 2H, Ar), 8.09 (bs, 1H, NH), 8.18 (s, 1H, Ar), 8.42 (d, J 8.2 Hz, 1H, Ar).

M/Z (M+H)⁺: 489.3

### Example 117: 3-(6-(2,2-dimethyl-3-oxopiperazine-1-carbonyl)-1-isobutyl-1H-pyrrolo[2,3-b]pyridin-3-yl)benzonitrile

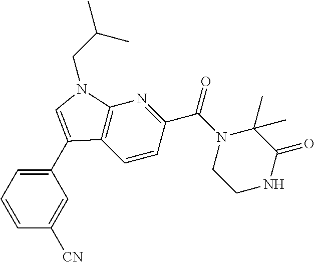 [SMILES: CC(C)Cn1cc(-c2cccc(C#N)c2)c2ccc(C(=O)N3CCNC(=O)C3(C)C)nc21]

Example 117 was prepared according to general procedure (XVIIa) starting from Compound 99 (100 mg) and (3-cyanophenyl)boronic acid (70 mg, 2 equiv). The crude was purified by flash chromatography (CyHex 100% to CyHex/EtOAc 80:20) then recrystallized from hot ACN (5 mL) to obtain Example 117 (43 mg, 41%) as a white solid.

¹H-NMR (DMSO-d₆, 300 MHz) δ: 0.88 (d, J 6.6 Hz, 6H, CH(CH₃)₂); 1.72 (s, 6H, C(CH₃)₂); 2.15-2.27 (m, 1H, CH(CH₃)₂); 3.50-3.58 (m, 2H, NCH₂); 4.12 (d, J 7.2 Hz, 2H, CH₂CH), 7.47 (d, J 8.2 Hz, 1H, Ar), 7.63-7.70 (m, 1H, Ar), 7.70-7.74 (m, 1H, Ar), 8.08-8.12 (m, 2H, Ar+NH), 8.19 (s, 1H, Ar), 8.33 (s, 1H, Ar), 8.52 (d, J 8.2 Hz, 1H, Ar). 2 protons not observed.

M/Z (M+H)⁺: 430.3.

### Example 118: 4-(1-isobutyl-3-(m-tolyl)-1H-pyrrolo[2,3-b]pyridine-6-carbonyl)-3,3-dimethylpiperazin-2-one

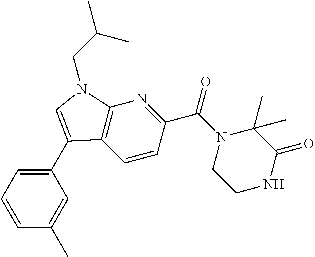 [SMILES: Cc1cccc(-c2cn(CC(C)C)c3nc(C(=O)N4CCNC(=O)C4(C)C)ccc23)c1]

Example 118 was prepared according to general procedure (XVIIa) starting from Compound 99 (100 mg) and (3-methylphenyl)boronic acid (65 mg, 2 equiv). The crude was purified by flash chromatography (CyHex 100% to CyHex/EtOAc 80:20) then recrystallized from hot ACN (5 mL) to obtain Example 118 (21 mg, 20%) as a beige solid.

¹H-NMR (DMSO-d₆, 400 MHz) δ: 0.88 (d, J 6.7 Hz, 6H, CH(CH₃)₂); 1.72 (s, 6H, C(CH₃)₂); 2.18-2.28 (m, 1H, CH(CH₃)₂); 2.38 (s, 3H, ArCH₃); 3.33-3.37 (m, 2H, NCH₂) 3.53-3.58 (m, 2H, NCH₂); 4.12 (d, J 7.3 Hz, 2H, CH₂CH), 7.09 (d, J 7.5 Hz, 1H, Ar), 7.34 (t, J 7.5 Hz, 1H, Ar), 7.44 (d, J 8.2 Hz, 1H, Ar), 7.51 (d, J 7.5 Hz, 1H, Ar), 7.54 (s, 1H, Ar), 8.05-8.11 (m, 2H, Ar+NH), 8.41 (d, J 8.2 Hz, 1H, Ar).

M/Z (M+H)⁺: 419.3

### Compound 262: 3-bromo-1,2-dimethyl-1H-pyrrolo[2,3-b]pyridine-6-carbonitrile

Compound 262 was prepared according to general procedure (XIII) in DMA from Compound 29 (300 mg) and iodomethane (198 mg, 1.1 equiv). The reaction mixture was stirred for 20 hours. The crude was purified by flash chromatography (CyHex 100% to CyHex/EtOAc 90:10) to obtain Compound 262 (94 mg, 30%) as a yellow solid.

M/Z (M[⁷⁹Br]+H)⁺: 252.1

### Compound 263: 3-(4-chloro-3-fluorophenyl)-1,2-dimethyl-1H-pyrrolo[2,3-b]pyridine-6-carbonitrile

Compound 263 was prepared according to general procedure (XVIIa) starting from Compound 262 (102 mg) and (4-chloro-3-fluorophenyl)boronic acid (107 mg, 1.5 equiv). The crude residue was purified by flash chromatography (Interchim® 50 μm, CyHex 100% to CyHex/EtOAc 80:20) to obtain Compound 263 (77 mg, 63%) as a yellow solid.

M/Z (M+H)⁺: 300.1

### Compound 264: 3-(4-chloro-3-fluorophenyl)-1,2-dimethyl-1H-pyrrolo[2,3-b]pyridine-6-carboxamide

Compound 264 was prepared according to general procedure (VII) starting from Compound 263 (77 mg) to obtain Compound 264 (83 mg) as a yellow solid.

M/Z (M[³⁵Cl]+H)⁺: 318.2.

### Compound 265: methyl 3-(4-chloro-3-fluorophenyl)-1,2-dimethyl-1H-pyrrolo[2,3-b]pyridine-6-carboxylate

Compound 265 was prepared according to general procedure (VIII) starting from Compound 264 (83 mg). The crude was purified by flash chromatography (CyHex 100% to CyHex/EtOAc 70:30) to obtain Compound 265 (54 mg, 62%) as a beige solid.

M/Z (M[³⁵Cl]+H)⁺: 333.1

### Compound 266: lithium 3-(4-chloro-3-fluorophenyl)-1,2-dimethyl-1H-pyrrolo[2,3-b]pyridine-6-carboxylate

Compound 266 was obtained (61 mg, n.d.) as a yellow solid according to general procedure (IXb) starting from Compound 265 (54 mg).

M/Z (M[³⁵Cl]+H)⁺: 333.2

### Compound 267: methyl 6-(4-(3-(4-chloro-3-fluorophenyl)-1,2-dimethyl-1H-pyrrolo[2,3-b]pyridine-6-carbonyl)-3,3-dimethylpiperazin-1-yl)-2,4-dimethylnicotinate

Compound 267 was prepared according to general procedure (Xc) starting from Compound 266 (52 mg), Compound 95 (55 mg) and N,N-diisopropylethylamine (4.0 equiv). The crude residue was purified by flash chromatography (CyHex 100% to CyHex/EtOAc 60:40) to obtain Compound 267 (54 mg, 58% over 2 steps) as a white solid.

M/Z (M[³⁵Cl]+H)⁺: 578.2

### Example 119: 6-(4-(3-(4-chloro-3-fluorophenyl)-1,2-dimethyl-1H-pyrrolo[2,3-b]pyridine-6-carbonyl)-3,3-dimethylpiperazin-1-yl)-2,4-dimethylnicotinic acid

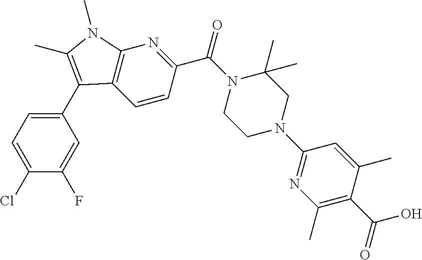 [SMILES: Cc1cc(N2CCN(C(=O)c3ccc4c(-c5ccc(Cl)c(F)c5)c(C)n(C)c4n3)C(C)(C)C2)nc(C)c1C(=O)O]

Example 119 was prepared according to general procedure (XIb) starting from Compound 267 (54 mg). The crude was triturated in MeOH (2*2 mL) and in Et₂O (2*2 mL) to obtain Example 119 (26 mg, 49%) as a white solid.

¹H-NMR (DMSO-d₆, 300 MHz) δ: 1.54 (s, 6H, 2 CH₃); 2.27 (s, 3H, Ar—CH₃); 2.38 (s, 3H, Ar—CH₃); 2.57 (s, 3H, Ar—CH₃); 3.51-3.56 (m, 2H, CH₂); 3.77-3.82 (m, 2H, CH₂); 3.84 (s, 3H, N—CH₃); 3.92 (s, 2H, CH₂); 6.35 (s, 1H, Ar); 7.31 (d, J 8.0 Hz, 1H, Ar); 7.34-7.37 (m, 1H, Ar); 7.51 (dd, J 10.7, 1.8 Hz, 1H, Ar); 7.69 (t, J 8.3 Hz, 1H, Ar); 8.04 (d, J 8.0 Hz, 1H, Ar); 12.66 (s, 1H, COOH).

M/Z (M[³⁵Cl]+H)⁺: 564.2.

Mp: 140-153° C.

### Compound 268: 3-bromo-1-(pyridin-4-ylmethyl)-1H-pyrrolo[2,3-b]pyridine-6-carbonitrile

To a solution of Compound 23 (300 mg, 1.0 equiv) and 4-(bromomethyl)pyridine hydrobromide (410 mg, 1.2 equiv) in THF (10 mL) was added sodium hydride (189 mg, 60% Wt, 3.5 equiv) at 0° C. The reaction mixture was stirred at 0° C. for 10 min then at 25° C. for 14 hours.

The reaction mixture was quenched with NH₄Cl sat. aq. (40 mL) then extracted with DCM (2*30 mL). The organic layer was washed with brine (20 mL), dried over magnesium sulfate then concentrated to dryness to obtain Compound 268 as a dark orange powder (530 mg).

M/Z (M[⁸¹Br]+H)⁺: 315.0.

### Compound 269: 3-bromo-1-(pyridin-4-ylmethyl)-1H-pyrrolo[2,3-b]pyridine-6-carboxamide

Compound 269 was prepared according to general procedure (VII) starting from Compound 268 (1.35 mmol) and using potassium carbonate (2.1 equiv) and hydrogen peroxide aqueous 30% (3.6 equiv). The crude was purified by flash chromatography (Merck 60®, CyHex/EtOAc 50:50 to EtOAc 100%, then DCM 100% to DCM/MeOH 80:20) to obtain Compound 269 (253 mg, 57% over 2 steps) as a white solid.

M/Z (M[⁸¹Br]+H)⁺: 333.1.

### Compound 270: methyl 3-bromo-1-(pyridin-4-ylmethyl)-1H-pyrrolo[2,3-b]pyridine-6-carboxylate

Compound 270 was prepared according to general procedure (VIII) starting from Compound 269 (253 mg). The crude was purified by flash chromatography (CyHex 100% to CyHex/EtOAc 0:100) to obtain Compound 270 (180 mg, 68%) as a white solid.

M/Z (M[⁸¹Br]+H)⁺: 348.1

### Compound 271: Lithium 3-bromo-1-(pyridin-4-ylmethyl)-1H-pyrrolo[2,3-b]pyridine-6-carboxylate

Compound 271 was prepared according to general procedure (IXb) starting from Compound 270 (180 mg) to obtain Compound 271 (220 mg) as a white solid.

M/Z (M[⁸¹Br]+H)⁺: 334.2.

### Compound 272: 4-(3-bromo-1-(pyridin-4-ylmethyl)-1H-pyrrolo[2,3-b]pyridine-6-carbonyl)-3,3-dimethylpiperazin-2-one

Compound 272 was prepared according to general procedure (Xb) starting from Compound 271 (260 μmol), 3,3-dimethylpiperazin-2-one (40 mg) and Et₃N (2.8 equiv). The crude residue was purified by flash chromatography (DCM 100% to DCM/MeOH 85:15) to obtain Compound 272 (140 mg) as a white solid. M/Z (M[⁷⁹Br]+H)⁺: 442.2

### Example 120: 4-(3-(4-chloro-3-fluorophenyl)-1-(pyridin-4-ylmethyl)-1H-pyrrolo[2,3-b]pyridine-6-carbonyl)-3,3-dimethylpiperazin-2-one

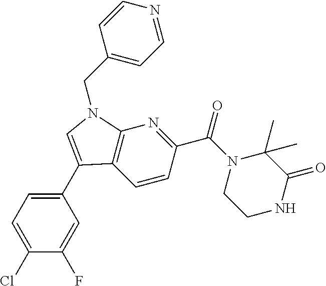 [SMILES: CC1(C)C(=O)NCCN1C(=O)c1ccc2c(-c3ccc(Cl)c(F)c3)cn(Cc3ccncc3)c2n1]

Example 120 was prepared according to general procedure (XVIIa) starting from Compound 272 (260 μmol) and (4-chloro-3-fluorophenyl)boronic acid (66 mg). The crude was purified by flash chromatography (Interchim® 50 μm, DCM to DCM/MeOH 80:20), then further purified by preparative HPLC (Column B, 5% to 100% ACN/H₂O), then freeze-dried with water to obtain Example 120 (35 mg, 27% over 3 steps) as a white solid.

¹H-NMR (DMSO-d₆, 300 MHz) δ: 1.67 (s, 6H, 2 CH₃); 1.92-2.99 (m, 2H, CH₂); 5.61 (s, 2H, CH₂); 7.12-7.14 (m, 2H, Ar); 7.48 (d, J 8.1 Hz, 1H, Ar); 7.61-7.69 (m, 2H, Ar), 7.79 (dd, J 1.2, 11.4 Hz, 1H, Ar); 7.99-8.05 (m, 1H, NH); 8.38 (s, 1H, Ar); 8.44 (d, J 5.7 Hz, 2H, Ar); 8.68 (d, J 8.1 Hz, 1H, Ar). 2 protons not observed.

M/Z (M[³⁵Cl]+H)⁺: 492.3

### Compound 273: 6-chloro-3-(4-chloro-3-fluorophenyl)-1-(1-methylcyclobutyl)-1H-pyrrolo[2,3-b]pyridine

Compound 273 was prepared according to general procedure (IV) starting from Compound 3 (500 mg) and 1-methylcyclobutan-1-amine (334 mg, 2.5 equiv) in DMA. The crude was purified by flash chromatography (Interchim® 50 μm, CyHex 100% to CyHex/Et₂O 90:10) to obtain Compound 273 (358 mg) as a yellow oil.

¹H-NMR (DMSO-d₆, 300 MHz) δ: 1.70 (s, 3H, CH₃); 1.84-2.10 (m, 2H, CH₂); 2.38-2.44 (m, 2H, CH₂); 2.69-2.80 (m, 2H, CH₂); 3.00 (s, 2H, CH₂); 7.23 (d, J 8.1 Hz, 1H Ar); 7.57-7.62 (m, 2H, Ar); 7.76-7.80 (m, 1H, Ar); 8.06 (s, 1H, Ar); 8.37 (d, J 8.1 Hz, 1H, Ar)

M/Z (M[³⁵Cl₂]+H)⁺: N.D.

### Compound 274: 3-(4-chloro-3-fluorophenyl)-1-(1-methylcyclobutyl)-1H-pyrrolo[2,3-b]pyridine-6-carbonitrile

Compound 274 was prepared according to general procedure (Va) starting from Compound 273 (358 mg). After 1 h at 110° C., Pd(PPh₃)₄ (118 mg, 0.1 equiv) was added and the reaction was further subjected to microwave irradiation at 150° C. for 30 min. The crude was purified by flash chromatography (CyHex 100% to CyHex/EtOAc 90:10) to obtain Compound 274 (180 mg, 36% over 2 steps) as a white solid.

M/Z (M[³⁵Cl]+H)⁺: 340.7

### Compound 275: 3-(4-chloro-3-fluorophenyl)-1-(1-methylcyclobutyl)-1H-pyrrolo[2,3-b]pyridine-6-carboxamide

To a solution of Compound 274 (180 mg) in DMSO (8 mL) was added K₂CO₃ (146 mg, 2.0 equiv) and H₂O₂ (162 μL, 30% Wt in H₂O, 3.0 equiv). The reaction was stirred at 25° C. for 18 hour. The reaction mixture was quenched with water (100 mL), the precipitate formed was filtered. The solid was washed with water and dried to obtain Compound 275 (175 mg) as a light-yellow solid.

M/Z (M[³⁵Cl]+H)⁺: 358.2.

### Compound 276: methyl 3-(4-chloro-3-fluorophenyl)-1-(1-methylcyclobutyl)-1H-pyrrolo[2,3-b]pyridine-6-carboxylate

Compound 276 was prepared according to general procedure (VIII) starting from Compound 275 (0.48 mmol). The crude was purified by flash chromatography (CyHex 100% to CyHex/EtOAc 70:30) to obtain Compound 276 (153 mg, 77% over 2 steps) as a yellow solid.

M/Z (M[³⁵Cl]+H)⁺: 373.2

### Compound 277: 3-(4-chloro-3-fluorophenyl)-1-(1-methylcyclobutyl)-1H-pyrrolo[2,3-b]pyridine-6-carboxylic acid

To a solution of Compound 276 (150 mg, 1.0 equiv) in mixture THF (20 mL)/water (10 mL) was added Lithium hydroxide monohydrate (51 mg, 3.0 equiv). The reaction was stirred at 25° C. for 48 hour. The solvent was concentrated to dryness and the residue was solubilized in water (100 mL). The aqueous solution was washed with Et₂O (2×50 mL). 1M aqueous HCl was then added to until pH<3. The precipitate was filtered and the solid was washed with water (2×10 mL) then dried to obtain Compound 277 (138 mg, 96%) as a light-yellow solid. M/Z (M[³⁵Cl]+H)⁺: 359.2.

### Compound 278: methyl 6-(4-(3-(4-chloro-3-fluorophenyl)-1-(1-methylcyclobutyl)-1H-pyrrolo[2,3-b]pyridine-6-carbonyl)-3,3-dimethylpiperazin-1-yl)-2,4-dimethylnicotinate

Compound 278 was prepared according to general procedure (Xa) starting from Compound 277 (60 mg) and Compound 95 (58 mg) and using N,N-diisopropylethylamine (4.0 equiv). The crude residue was purified by flash chromatography (CyHex 100% to CyHex/EtOAc 70:30) to obtain Compound 278 (92 mg, 89%) as a white solid.

M/Z (M[³⁵Cl]+H)⁺: 618.6

### Example 121: 6-(4-(3-(4-chloro-3-fluorophenyl)-1-(1-methylcyclobutyl)-1H-pyrrolo[2,3-b]pyridine-6-carbonyl)-3,3-dimethylpiperazin-1-yl)-2,4-dimethylnicotinic acid

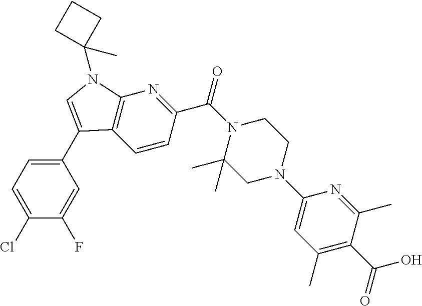 [SMILES: Cc1cc(N2CCN(C(=O)c3ccc4c(-c5ccc(Cl)c(F)c5)cn(C5(C)CCC5)c4n3)C(C)(C)C2)nc(C)c1C(=O)O]

Example 121 was prepared according to general procedure (XIb) starting from Compound 278 (80 mg). The crude residue was purified by flash chromatography (DCM 100% to DCM/MeOH 80:20), then freeze-dried with water to obtain Example 121 (61 mg, 78%) as a white solid.

¹H-NMR (DMSO-d₆, 300 MHz) δ: 1.51 (s, 6H, (CH₃)₂); 1.73 (s, 3H, CH₃); 1.89-1.96 (m, 1H, CH); 2.02-2.11 (m, 1H, CH); 2.23 (s, 3H, CH₃); 2.35 (s, 3H, CH₃); 2.71-2.79 (m, 2H, CH₂); 3.36-3.40 (m, 2H, CH₂); 3.50-3.55 (m, 2H, CH₂); 3.78-3.84 (m, 2H, CH₂); 3.88 (s, 2H, CH₂); 6.27 (s, 1H, Ar); 7.40 (d, J 8.4 Hz, 1H, Ar); 7.58-7.67 (m, 2H, Ar); 7.80 (d, J 10.2 Hz, 1H, Ar); 8.15 (s, 1H, Ar); 8.42 (d, J 8.4 Hz, 1H, Ar). 1 proton not observed.

M/Z (M[³⁵Cl]+H)⁺: 604.6.

### Compound 279: methyl 6-(4-(3-(4-chloro-3-fluorophenyl)-1-methyl-1H-pyrrolo[2,3-b]pyridine-6-carbonyl)-3,3-dimethylpiperazin-1-yl)-2,4-dimethylnicotinate

Compound 279 was prepared according to general procedure (Xa) starting from Compound 84 (60 mg) and Compound 78 (68 mg, 1.1 eq) and using N,N-diisopropylethylamine (4.0 equiv). The crude residue was purified by flash chromatography (CyHex 100% to EtOAc 100%) to obtain Compound 279 (92 mg, 83%) as a clear oil.

M/Z (M[³⁵Cl]+H)⁺: 564.4

### Example 122: 6-(4-(3-(4-chloro-3-fluorophenyl)-1-methyl-1H-pyrrolo[2,3-b]pyridine-6-carbonyl)-3,3-dimethylpiperazin-1-yl)-2,4-dimethylnicotinic acid hydrochloride

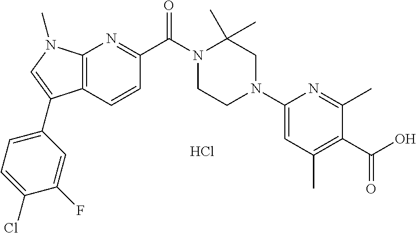 [SMILES: Cc1cc(N2CCN(C(=O)c3ccc4c(-c5ccc(Cl)c(F)c5)cn(C)c4n3)C(C)(C)C2)nc(C)c1C(=O)O ; Cl]

Example 122 was prepared according to general procedure (XIb) starting from Compound 279 (92 mg). The crude was purified by preparative HPLC (Column B, 5% to 100% ACN/H₂O), then freeze-dried with HCl in water to obtain Example 122 (75 mg, 78%) as a yellow solid.

¹H-NMR (DMSO-d₆, 300 MHz) δ:1.56 (s, 6H, (CH₃)₂); 2.31 (s, 3H, CH₃); 2.44 (s, 3H, CH₃); 3.80-3.83 (m, 4H, 2(CH₂)); 3.90 (s, 3H, CH₃); 3.90-3.94 (m, 2H, CH₂); 6.57 (bs, 1H, Ar); 7.38 (d, J 8.4 Hz, 1H, Ar); 7.56-7.66 (m, 2H, Ar); 7.72-7.77 (m, 1H, Ar); 8.22 (s, 1H, Ar); 8.46 (d, J 8.4 Hz, 1H, Ar). 2 protons not observed.

M/Z (M[³⁵Cl]+H)⁺: 550.4

### Compound 280: tert-butyl 4-carbamoylpiperidine-1-carboxylate

To a solution of 1-(tert-butoxycarbonyl)piperidine-4-carboxylic acid (600 mg, 2.62 mmol) in ammonia 0.5M in dioxane (26.2 mL, 5 equiv) was added ((1H-benzo[d][1,2,3]triazol-1-yl)oxy)tris(dimethylamino)phosphonium hexafluorophosphate(V) (1.50 g, 1.3 equiv). The reaction was stirred at 25° C. for 1 hour. The reaction mixture was quenched with NH4Cl sat. (40 mL) then extracted with EtOAc (2*40 mL). The organic layer was washed with brine (40 mL), dried over magnesium sulfate then concentrated to dryness. The crude was purified by flash chromatography (DCM 100% to DCM/MeOH 95:5) to obtain Compound 280 as a white solid (825 mg).

M/Z (M-tBu+H)⁺: 173.1.

### Compound 281: methyl 6-(1-(tert-butoxycarbonyl)piperidine-4-carboxamido)-2,4-dimethylnicotinate

Under inert atmosphere, to a solution of methyl 6-chloro-2,4-dimethylnicotinate (525 mg, 2.63 mmol) and Compound 280 (600 mg, 1.0 equiv) in dioxane (0.1 M) was added Cesium Carbonate (1.71 g, 2.0 equiv). The mixture was degassed for 10 min before addition of XantPhos PdG4 (253 mg, 0.1 Eq). The reaction mixture was heated at 90° C. for 18 hours. The reaction mixture was filtered through a pad of Celite, the pad was washed with EtOAc then the filtrate was concentrated to dryness. The crude residue was purified by flash chromatography (CyHex 100% to CyHex/EtOAc 60:40) to obtain Compound 281 as a clear oil (740 mg).

M/Z (M+H)⁺: 392.3

### Compound 282: methyl 2,4-dimethyl-6-(piperidine-4-carboxamido)nicotinate hydrochloride

A solution of Compound 281 (200 mg) in Et₂O (10 mL) was treated with HCl 2M in Et₂O (5.36 mL, 21 eq) for 20 hours. The reaction mixture was concentrated under reduced pressure then the residue was suspended in Et₂O (20 mL) and concentrated under reduced pressure to obtain Compound 282 (190 mg) as a green solid.

M/Z (M+H)⁺: 292.2

### Compound 283: methyl 6-(1-(1-(tert-butyl)-3-(4-chloro-3-fluorophenyl)-1H-pyrrolo[2,3-b]pyridine-6-carbonyl)piperidine-4-carboxamido)-2,4-dimethylnicotinate

Compound 283 was prepared according to general procedure (Xa) starting from Compound 78 (60 mg) and Compound 282 (62 mg, 1.1 equiv), and using N,N-diisopropylethylamine (4.0 equiv). The crude was purified by flash chromatography (Merck 60®, CyHex 100% to CyHex/EtOAc 55:45) to obtain Compound 283 (92 mg, 86%) as a white solid.

M/Z (M[³⁵Cl]+H)⁺: 620.6.

### Example 123: 6-(1-(1-(tert-butyl)-3-(4-chloro-3-fluorophenyl)-1H-pyrrolo[2,3-b]pyridine-6-carbonyl)piperidine-4-carboxamido)-2,4-dimethylnicotinic acid

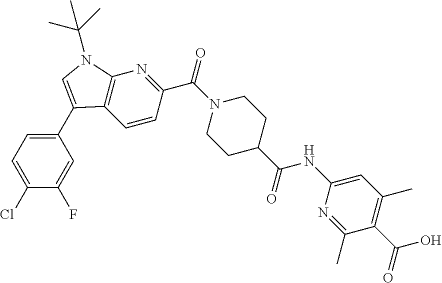 [SMILES: Cc1cc(NC(=O)C2CCN(C(=O)c3ccc4c(-c5ccc(Cl)c(F)c5)cn(C(C)(C)C)c4n3)CC2)nc(C)c1C(=O)O]

Example 123 was prepared according to general procedure (XIb) starting from Compound 283 (92 mg). The crude was purified by preparative HPLC (Column B, 40% ACN/H₂O), then freeze-dried with MeOH/H₂O to obtain Example 123 (33 mg, 37%) as a white solid.

¹H-NMR (DMSO-d₆, 300 MHz) δ: 1.52-1.74 (m, 2H, CH₂); 1.80 (s, 9H, C(CH₃)₃); 1.90-2.08 (m, 2H, CH₂); 2.28 (s, 3H, CH₃); 2.40 (s, 3H, CH₃); 2.78-2.98 (m, 2H, N—CHₐH_{b}+CH—CONH); 3.05-3.19 (m, 1H, N—CHₐH_{b}); 4.01-4.18 (m, 1H, N—CHₐH_{b}); 4.52-4.67 (m, 1H, N—CHₐH_{b}) 7.43 (d, J 8.3 Hz, 1H, Ar); 7.59-7.70 (m, 2H, 2 Ar); 7.82 (dd, J 11.1 Hz, 1.7 Hz, 1H, Ar); 7.85 (s, 1H, Ar); 8.21 (s, 1H, Ar); 8.43 (d, J 8.3 Hz, 1H, Ar); 10.54 (s, 1H, CO—NH). 3 protons not observed.

M/Z (M[³⁵Cl]+H)⁺: 606.5.

### Compound 284: methyl 2-((1R,5S,6s)-3-(1-(tert-butyl)-3-(4-chloro-3-fluorophenyl)-1H-pyrrolo[2,3-b]pyridine-6-carbonyl)-3-azabicyclo[3.1.0]hexan-6-yl)acetate

Compound 284 was prepared according to general procedure (Xa) starting from Compound 78 (70 mg) and methyl 2-((1R,5S,6s)-3-azabicyclo[3.1.0]hexan-6-yl)acetate hydrochloride (43 mg, 1.1 equiv), and using N,N-diisopropylethylamine (4.0 equiv). The crude was purified by flash chromatography (Merck 60®, CyHex 100% to CyHex/EtOAc 60:40) to obtain Compound 284 (105 mg) as a white solid.

M/Z (M[³⁵Cl]+H)⁺: 484.3

### Example 124: 2-((1R,5S,6s)-3-(1-(tert-butyl)-3-(4-chloro-3-fluorophenyl)-1H-pyrrolo[2,3-b]pyridine-6-carbonyl)-3-azabicyclo[3.1.0]hexan-6-yl)acetic acid

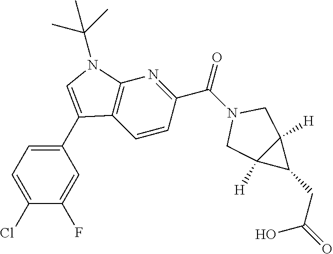 [SMILES: CC(C)(C)n1cc(-c2ccc(Cl)c(F)c2)c2ccc(C(=O)N3C[C@@H]4[C@@H](CC(=O)O)[C@@H]4C3)nc21]

Example 124 was prepared according to general procedure (IXa) starting from Compound 284 (98 mg). The residue was freeze-dried with MeOH/H₂O to obtain Example 124 (60 mg, 63% over 2 steps) as a white solid.

¹H-NMR (DMSO-d₆, 300 MHz) δ: 0.70-0.79 (m, 1H, CH); 1.46-1.58 (m, 2H, 2 CH); 1.81 (s, 9H, C(CH₃)₃); 2.15-2.30 (m, 2H, CH₂—COOH); 3.50 (dd, J 12.2 Hz, 3.8 Hz, 1H, N—CHₐH_{b}); 3.91 (s, 2H, N—CH₂); 4.01 (d, J 12.2 Hz, 1H, N—CHₐH_{b}); 7.57 (d, J 8.3 Hz, 1H, Ar); 7.62-7.67 (m, 2H, Ar); 7.82 (dd, J 11.1 Hz, 1.7 Hz, 1H, Ar); 8.23 (s, 1H, Ar); 8.41 (d, J 8.3 Hz, 1H, Ar); 12.06 (bs, 1H, COOH).

M/Z (M[³⁵Cl]+H)⁺: 470.2

### Compound 285: methyl 2-(1-(1-(tert-butyl)-3-(4-chloro-3-fluorophenyl)-1H-pyrrolo[2,3-b]pyridine-6-carbonyl)piperidin-4-yl)acetate

Compound 285 was prepared according to general procedure (Xa) starting from Compound 78 (150 mg) and methyl 2-(piperidin-4-yl)acetate (75 mg, 1.1 equiv), and using N,N-diisopropylethylamine (4.0 equiv). The crude was purified by flash chromatography (Merck 60®, CyHex 100% to CyHex/EtOAc 50:50) to obtain Compound 285 (153 mg, 73%) as a white solid.

M/Z (M[³⁵Cl]+H)⁺: 486.3

### Example 125: 2-(1-(1-(tert-butyl)-3-(4-chloro-3-fluorophenyl)-1H-pyrrolo[2,3-b]pyridine-6-carbonyl)piperidin-4-yl)acetic acid

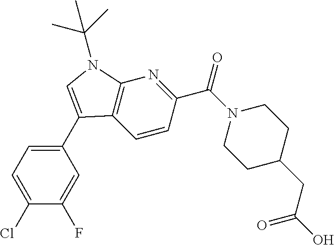 [SMILES: CC(C)(C)n1cc(-c2ccc(Cl)c(F)c2)c2ccc(C(=O)N3CCC(CC(=O)O)CC3)nc21]

Example 125 was prepared according to general procedure (IXa) starting from Compound 285 (98 mg). The residue was freeze-dried with MeOH/H₂O to obtain Example 125 (79 mg, 54%) as a white solid.

¹H-NMR (DMSO-d₆, 300 MHz) δ: 1.11-1.38 (m, 2H, CH₂); 1.58-1.74 (m, 1H, CH); 1.79 (s, 9H, C(CH₃)₃); 1.92-2.09 (m, 2H, CH₂); 2.14-2.27 (m, 2H, CH₂—COOH); 2.85 (t, J 12.2 Hz, 1H, N—CHₐH_{b}); 3.1 (t, J 12.2 Hz, 1H, N—CHₐH_{b}); 4.0 (d, J 12.2 Hz, 1H, N—CHₐH_{b}); 4.52 (d, J 12.2 Hz, 1H, N—CHₐH_{b}); 7.40 (d, J 8.3 Hz, 1H, Ar); 7.57-7.71 (m, 2H, 2 Ar); 7.81 (dd, J 11.1 Hz, 1.7 Hz, 1H, Ar); 8.19 (s, 1H, Ar); 8.41 (d, J 8.3 Hz, 1H, Ar). 1 proton not observed.

M/Z (M[³⁵Cl]+H)⁺: 472.2

### Compound 286: methyl 6-(4-(3-(4-chloro-3-fluorophenyl)-1-(1-ethoxy-2-methylpropan-2-yl)-1H-pyrrolo[2,3-b]pyridine-6-carbonyl)-3,3-dimethylpiperazin-1-yl)-2,4-dimethylnicotinate

Compound 286 was prepared according to general procedure (XIII) in DMA starting from Compound 254 (106 mg) and iodoethane (18 μL, 1.3 equiv). The crude was purified by flash chromatography (CyHex 100% to CyHex/EtOAc 70:30) to obtain Compound 286 (119 mg) as a yellow oil.

M/Z (M[³⁵Cl]+H)⁺: 650.3

### Example 126: 6-(4-(3-(4-chloro-3-fluorophenyl)-1-(1-ethoxy-2-methylpropan-2-yl)-1H-pyrrolo[2,3-b]pyridine-6-carbonyl)-3,3-dimethylpiperazin-1-yl)-2,4-dimethylnicotinic acid

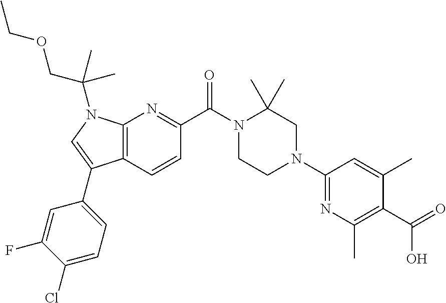 [SMILES: CCOCC(C)(C)n1cc(-c2ccc(Cl)c(F)c2)c2ccc(C(=O)N3CCN(c4cc(C)c(C(=O)O)c(C)n4)CC3(C)C)nc21]

Example 126 was prepared according to general procedure (XIb) starting from Compound 286 (119 mg). The crude was purified by preparative HPLC (40% to 80% ACN/H₂O), then freeze-dried with water to obtain Example 126 (64 mg, 55%) as a white solid.

¹H-NMR (DMSO-d₆, 300 MHz) δ: 0.98 (t, J 7.0 Hz, 3H, CH₂—CH₃); 1.56 (s, 6H, 2 CH₃); 1.78 (s, 6H, 2 CH₃); 2.30 (s, 3H, Ar—CH₃); 2.43 (s, 3H, Ar—CH₃); 3.56-3.62 (m, 2H, CH₂); 3.83-3.89 (m, 2H, CH₂); 3.95 (s, 2H, CH₂); 4.03 (m, 2H, EtO—CH₂); 6.50 (bs, 1H, Ar); 7.41 (d, J 8.3 Hz, 1H, Ar); 7.60-7.67 (m, 2H, Ar); 7.79-7.83 (m, 1H, Ar); 8.16 (s, 1H, Ar); 8.43 (d, J 8.3 Hz, 1H, Ar). 3 protons not observed.

M/Z (M[³⁵Cl]+H)⁺: 636.3

### Compound 287: tert-butyl 4-(5-(methoxycarbonyl)-4,6-dimethylpyridin-2-yl)-1,4-diazepane-1-carboxylate

Compound 287 was prepared according to general procedure (XXI) starting from methyl 6-chloro-2,4-dimethylnicotinate (300 mg, 1.50 mmol) and tert-butyl 1,4-diazepane-1-carboxylate (331 mg, 1.1 Eq). The crude was purified by flash chromatography (CyHex 100% to CyHex/EtOAc 67:33) to obtain Compound 287 as a yellow solid (425 mg).

M/Z (M+H)⁺: 363.4.

### Compound 288: methyl 6-(1,4-diazepan-1-yl)-2,4-dimethylnicotinate hydrochloride

A solution of Compound 287 (425 mg) in DCM (1.7 mL) was treated with HCl 2M in Et₂O (12.3 mL, 21 eq) for 4 hours. The reaction mixture was concentrated under reduced to obtain Compound 288 (340 mg) as a yellow solid.

M/Z (M+H)⁺: 264.2

### Compound 289: methyl 6-(4-(1-(tert-butyl)-3-(4-chloro-3-fluorophenyl)-1H-pyrrolo[2,3-b]pyridine-6-carbonyl)-1,4-diazepan-1-yl)-2,4-dimethylnicotinate

Compound 289 was prepared according to general procedure (Xa) starting from Compound 78 (60 mg) and Compound 288 (57 mg, 1.1 equiv), and using N,N-diisopropylethylamine (4.0 equiv). The crude was purified by flash chromatography (CyHex 100% to CyHex/EtOAc 80:20) to obtain Compound 289 (75 mg, 73%) as an off white solid.

M/Z (M[³⁵Cl]+H)⁺: 592.5.

### Example 127: 6-(4-(1-(tert-butyl)-3-(4-chloro-3-fluorophenyl)-1H-pyrrolo[2,3-b]pyridine-6-carbonyl)-1,4-diazepan-1-yl)-2,4-dimethylnicotinic acid

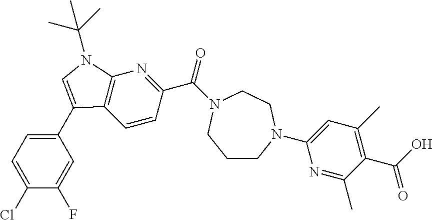 [SMILES: Cc1cc(N2CCCN(C(=O)c3ccc4c(-c5ccc(Cl)c(F)c5)cn(C(C)(C)C)c4n3)CC2)nc(C)c1C(=O)O]

Example 127 was prepared according to general procedure (XIb) starting from Compound 289 (73 mg). The crude was purified by flash chromatography (DCM 100% to DCM/MeOH 80:20) to obtain Example 127 (33 mg, 37%) as a white solid.

¹H-NMR (DMSO-d₆, 300 MHz) δ: 1.76 (s, 2.7H, one rotamer of C(CH₃)₃); 1.80 (s, 6.3H, other rotamer of C(CH₃)₃); 1.90-2.01 (m, 2H, CH₂); 2.04 (s, 2.1H, other rotamer of CH₃); 2.21 (s, 2.1H, other rotamer of CH₃); 2.24 (s, 0.9H, one rotamer of CH₃); 2.36 (s, 0.9H, one rotamer of CH₃); 3.56-3.64 (m, 2H, N—CH₂); 3.67-3.74 (m, 2H, N—CH₂); 3.75-3.82 (m, 2H, N—CH₂) 3.82-3.90 (m, 2H, N—CH₂); 6.34 (s, 0.7H, other rotamer of Ar); 6.42 (s, 0.3H, one rotamer of Ar); 6.75 (d, J 8.3 Hz, 0.7H, other rotamer of Ar); 7.32 (d, J 8.3 Hz, 0.3H, one rotamer of Ar); 7.58-7.71 (m, 2H, 2 Ar); 7.81 (d, J 11.1 Hz, 1H, Ar); 8.18 (s, 1H, Ar); 8.29 (d, J 8.3 Hz, 0.7H, other rotamer of Ar); 8.40 (d, J 8.3 Hz, 0.3H, one rotamer of Ar). 1 proton not observed.

M/Z (M[³⁵Cl]+H)⁺: 578.3.

### Compound 290: tert-butyl 7-(5-(methoxycarbonyl)-4,6-dimethylpyridin-2-yl)-2,7-diazaspiro[4.4]nonane-2-carboxylate

Compound 290 was prepared according to general procedure (XXI) starting from methyl 6-chloro-2,4-dimethylnicotinate (250 mg, 1.25 mmol) and tert-butyl 1,4-diazepane-1-carboxylate (312 mg, 1.1 Eq). The crude was purified by flash chromatography (CyHex 100% to CyHex/EtOAc 80:20) to obtain Compound 290 as a white solid (377 mg).

M/Z (M+H)⁺: 390.3

### Compound 291: methyl 2,4-dimethyl-6-(2,7-diazaspiro[4.4]nonan-2-yl)nicotinate hydrochloride

A solution of Compound 290 (377 mg) in DCM (10 mL) was treated with HCl 2M in Et₂O (10.2 mL, 21 eq) for 18 hours. The reaction mixture was concentrated under reduced to obtain Compound 291 (328 mg, 80% over 2 steps) as a white solid.

¹H-NMR (DMSO-d₆, 300 MHz) δ: 1.94-2.02 (m, 2H, N—CH₂—CH₂); 2.02-21 (m, 2H, N—CH₂—CH₂); 2.32 (s, 3H, CH₃); 2.49 (s, 3H, CH₃); 3.13-3.17 (m, 2H, N—CH₂—CH₂); 3.27-3.30 (m, 2H, N—CH₂); 3.84 (s, 3H, OCH₃); 6.67 (s, 1H, Ar); 9.61 (bs, 2H, NH₂Cl). Two N—CH₂ not observed.

### Compound 292: methyl 6-(7-(1-(tert-butyl)-3-(4-chloro-3-fluorophenyl)-1H-pyrrolo[2,3-b]pyridine-6-carbonyl)-2,7-diazaspiro[4.4]nonan-2-yl)-2,4-dimethylnicotinate

Compound 292 was prepared according to general procedure (Xa) starting from Compound 78 (60 mg) and Compound 291 (56 mg, 1.0 equiv), and using N,N-diisopropylethylamine (4.0 equiv). The crude was purified by flash chromatography (CyHex 100% to CyHex/EtOAc 70:30) to obtain Compound 292 (73 mg, 68%) as an off white solid.

M/Z (M[³⁵Cl]+H)⁺: 618.6.

### Example 128: 6-(7-(1-(tert-butyl)-3-(4-chloro-3-fluorophenyl)-1H-pyrrolo[2,3-b]pyridine-6-carbonyl)-2,7-diazaspiro[4.4]nonan-2-yl)-2,4-dimethylnicotinic acid

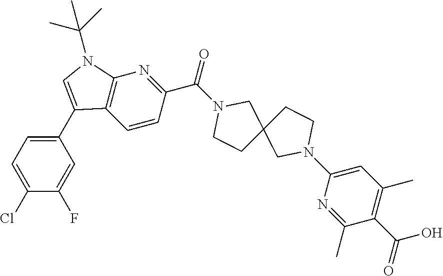 [SMILES: Cc1cc(N2CCC3(CCN(C(=O)c4ccc5c(-c6ccc(Cl)c(F)c6)cn(C(C)(C)C)c5n4)C3)C2)nc(C)c1C(=O)O]

Example 128 was prepared according to general procedure (XIb) starting from Compound 292 (70 mg). The crude was purified by flash chromatography (DCM 100% to DCM/MeOH 80:20) and freeze-dried from MeOH/water to obtain Example 128 (35 mg, 51%) as a white solid.

¹H-NMR (MeOD, 300 MHz) δ: 1.79 (s, 5.4H, one rotamer of C(CH₃)₃); 1.89 (s, 3.6H, other rotamer of C(CH₃)₃); 2.02-2.27 (m, 4H, 2 CH₂); 2.32 (s, 1.8H, one rotamer of CH₃); 2.37 (s, 1.2H, other rotamer of CH₃); 2.44 (s, 1.8H, one rotamer of CH₃); 2.48 (s, 1.2H, other rotamer of CH₃); 3.37-3.50 (m, 1H, one rotamer of N—CH₂); 3.50-3.58 (m, 1H, other rotamer of N—CH₂) 3.58-3.70 (m, 2H, N—CH₂); 3.70-3.84 (m, 1H, one rotamer of N—CH₂); 3.84-3.92 (m, 1H, other rotamer of N—CH₂); 3.92-3.99 (m, 0.6H, one rotamer of N—CH₂); 4.08-4.25 (m, 1.4H, other rotamer of N—CH₂); 6.31 (s, 0.6H, one rotamer of Ar); 6.40 (s, 0.4H, other rotamer of Ar); 7.44-7.61 (m, 3H, 3 Ar); 7.73 (d, J 8.3 Hz, 0.4H, one rotamer of Ar); 7.74 (d, J 8.3 Hz, 0.6H, other rotamer of Ar); 7.98 (s, 0.6H, one rotamer of Ar); 8.01 (s, 0.4H, other rotamer of Ar); 8.33 (d, J 8.3 Hz, 0.6H, one rotamer of Ar); 8.35 (d, J 8.3 Hz, 0.4H, other rotamer of Ar). 1 proton not observed.

M/Z (M[³⁵Cl]+H)⁺: 604.4.

### Compound 293: 3-bromo-1-ethyl-1H-pyrrolo[2,3-b]pyridine-6-carbonitrile

Compound 293 was prepared according to general procedure (XIII) in THE starting from Compound 23 (300 mg) and iodoethane (120 μL, 1.1 equiv). The crude was concentrated to obtain Compound 293 (374 mg) as an orange solid.

M/Z (M[⁷⁹Br]+H)⁺: 313.2

### Compound 294: 3-(4-chloro-3-fluorophenyl)-1-ethyl-1H-pyrrolo[2,3-b]pyridine-6-carbonitrile

Compound 294 was prepared according to general procedure (XVIIa) starting from Compound 293 (1.35 mmol) and (4-chloro-3-fluorophenyl)boronic acid (282 mg, 1.2 equiv). The crude was purified by flash chromatography (CyHex 100% to CyHex/EtOAc 80:20) to obtain Compound 294 (142 mg, 35% over 2 steps) as an off white solid.

M/Z (M[³⁵Cl]+H)⁺: 300.2

### Compound 295: 3-(4-chloro-3-fluorophenyl)-1-ethyl-1H-pyrrolo[2,3-b]pyridine-6-carboxamide

Compound 295 was prepared according to general procedure (VII) starting from Compound 294 (142 mg). The crude was purified by flash chromatography (CyHex 100% to EtOAc 100%) to obtain Compound 295 (148 mg, 98%) as an off white solid.

M/Z (M[³⁵Cl]+H)⁺: 318.2

### Compound 296: methyl 3-(4-chloro-3-fluorophenyl)-1-ethyl-1H-pyrrolo[2,3-b]pyridine-6-carboxylate

To a solution of Compound 295 (140 mg) in Methanol (20 mL) was added DMF-DMA (351 μL, 6 Eq). The reaction was heated at 60° C. for 18 hours. The reaction mixture was concentrated in vacuo, the residue was hydrolyzed with water (150 mL) then the solid formed was filtered to obtain Compound 296 (141 mg, 96%) as a yellow solid.

M/Z (M[³⁵Cl]+H)⁺: 333.2.

### Compound 297: 3-(4-chloro-3-fluorophenyl)-1-ethyl-1H-pyrrolo[2,3-b]pyridine-6-carboxylic acid

Compound 297 was obtained (120 mg, 89%) as a white solid according to general procedure (IXa) starting from Compound 296 (140 mg).

M/Z (M[³⁵Cl]+H)⁺: 319.2

### Compound 298: methyl 6-(4-(3-(4-chloro-3-fluorophenyl)-1-ethyl-1H-pyrrolo[2,3-b]pyridine-6-carbonyl)-3,3-dimethylpiperazin-1-yl)-2,4-dimethylnicotinate

Compound 298 was prepared according to general procedure (Xa) starting from Compound 297 (60 mg) and Compound 95 (65 mg, 1.1 equiv), and using N,N-diisopropylethylamine (4.0 equiv). The crude was purified by flash chromatography (CyHex 100% to CyHex/EtOAc 65:35) to obtain Compound 298 (90 mg, 83%) as a white solid.

M/Z (M[³⁵Cl]+H)⁺: 578.4.

### Example 129: 6-(4-(3-(4-chloro-3-fluorophenyl)-1-ethyl-1H-pyrrolo[2,3-b]pyridine-6-carbonyl)-3,3-dimethylpiperazin-1-yl)-2,4-dimethylnicotinic acid

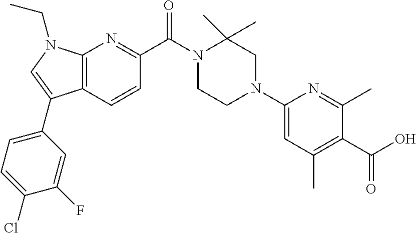 [SMILES: CCn1cc(-c2ccc(Cl)c(F)c2)c2ccc(C(=O)N3CCN(c4cc(C)c(C(=O)O)c(C)n4)CC3(C)C)nc21]

Example 129 was prepared according to general procedure (XIb) starting from Compound 298 (90 mg). The crude was purified by preparative HPLC (Column B, 5% to 100% ACN/H₂O), then freeze-dried with water and aqueous HCl (1M) to obtain Example 129 (25 mg, 27%) as a white solid.

¹H-NMR (DMSO-d₆, 300 MHz) δ: 1.47 (t, J 7.2 Hz, 3H, CH₃); 1.54 (s, 6H, (CH₃)₂); 2.27 (s, 3H, CH₃); 2.38 (s, 3H, CH₃); 3.52-3.55 (m, 2H, CH₂); 3.77-3.80 (m, 2H, CH₂); 3.91 (s, 2H, CH₂); 4.35 (q, J 7.2 Hz, 2H, CH₂); 6.34 (bs, 1H, Ar); 7.38 (d, J 8.4 Hz, 1H, Ar); 7.62-7.64 (m, 2H, Ar); 7.76 (dd, J 11.7, 1.5 Hz, 1H, Ar); 8.29 (s, 1H, Ar); 8.45 (d, J 8.4 Hz, 1H, Ar). 1 proton not observed.

M/Z (M[³⁵Cl]+H)⁺: 564.4.

### Compound 299: tert-butyl (R)-4-(5-(methoxycarbonyl)-4,6-dimethylpyridin-2-yl)-2-methylpiperazine-1-carboxylate

Compound 299 was prepared according to general procedure (XXI) starting from methyl 6-chloro-2,4-dimethylnicotinate (426 mg, 2.13 mmol) and tert-butyl (R)-2-methylpiperazine-1-carboxylate (470 mg, 1.1 Eq). The crude was purified by flash chromatography (CyHex 100% to CyHex/EtOAc 50:50) to obtain Compound 299 as a yellow gum (489 mg, 63%).

M/Z (M+H)⁺: 390.3

### Compound 300: methyl (R)-2,4-dimethyl-6-(3-methylpiperazin-1-yl)nicotinate hydrochloride

Compound 300 was prepared according to general procedure (XVIIIc) starting from Compound 299 (489 mg). The reaction mixture was concentrated under reduced to obtain Compound 300 (390 mg, 97%) as a beige solid.

M/Z (M+H)⁺: 264.2.

### Compound 301: methyl (R)-6-(4-(1-(tert-butyl)-3-(4-chloro-3-fluorophenyl)-1H-pyrrolo[2,3-b]pyridine-6-carbonyl)-3-methylpiperazin-1-yl)-2,4-dimethylnicotinate

Compound 301 was prepared according to general procedure (Xb) starting from Compound 78 (61 mg) and Compound 300 (70 mg, 1.0 equiv), and using N,N-diisopropylethylamine (4.0 equiv). The crude was purified by flash chromatography (CyHex 100% to EtOAc 100%) to obtain Compound 301 (114 mg, 97%) as an off white solid.

M/Z (M[³⁵Cl]+H)⁺: 592.4.

### Example 130: (R)-6-(4-(1-(tert-butyl)-3-(4-chloro-3-fluorophenyl)-1H-pyrrolo[2,3-b]pyridine-6-carbonyl)-3-methylpiperazin-1-yl)-2,4-dimethylnicotinic acid

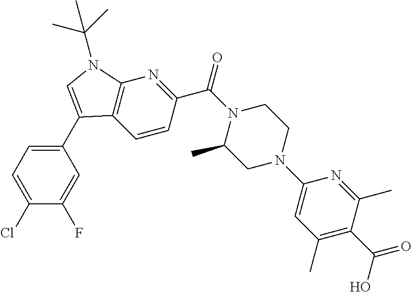 [SMILES: Cc1cc(N2CCN(C(=O)c3ccc4c(-c5ccc(Cl)c(F)c5)cn(C(C)(C)C)c4n3)[C@H](C)C2)nc(C)c1C(=O)O]

Example 130 was prepared according to general procedure (XIb) starting from Compound 301 (114 mg). The crude was purified by flash chromatography (DCM 100% to DCM/MeOH 80:20) and freeze-dried from MeOH/water to obtain Example 130 (77 mg, 69%) as a white solid.

¹H-NMR (DMSO-d₆, 300 MHz) δ: 1.25 (d, J 6.6 Hz, 3H, CH₃); 1.81 (s, 9H, C(CH₃)₃); 2.21 (s, 3H, CH₃); 2.32 (s, 3H, CH₃); 2.77-3.05 (m, 2H, CH₂); 3.94-4.86 (m, 5H, CH, 2*CH₂); 6.48 (s, 1H, Ar); 7.45 (d, 1H, J 8.1 Hz, Ar); 7.60-7.68 (m, 2H, Ar); 7.82 (dd, 1H, J 11.7, 1.8 Hz, Ar); 8.22 (s, 1H, Ar); 8.44 (d, 1H, J 8.1 Hz, Ar). 1 proton not observed.

M/Z (M[³⁵Cl]+H)⁺: 578.4

### Compound 302: 6-chloro-3-(4-chloro-3-fluorophenyl)-1-(2-(pyridin-2-yl)propan-2-yl)-1H-pyrrolo[2,3-b]pyridine

Compound 302 was prepared according to general procedure (IV) starting from Compound 3 (393 mg) and 2-(pyridin-2-yl)propan-2-amine (420 mg, 2.5 equiv). The crude was purified by flash chromatography (CyHex 100% to CyHex/EtOAc 90:10) to obtain Compound 302 (176 mg) as a yellow solid.

M/Z (M[³⁵Cl]+H)⁺: 400.1

### Compound 303: 3-(4-chloro-3-fluorophenyl)-1-(2-(pyridin-2-yl)propan-2-yl)-1H-pyrrolo[2,3-b]pyridine-6-carbonitrile

Compound 303 was prepared according to general procedure (Va) starting from Compound 302 (175 mg). The reaction mixture was filtered and restarted 3 times. The crude was purified by flash chromatography (CyHex 100% to CyHex/EtOAc 70:30) to obtain Compound 303 (61 mg, 13% over two steps) as a yellow solid.

M/Z (M[³⁵Cl]+H)⁺: 391.8

### Compound 304: 3-(4-chloro-3-fluorophenyl)-1-(2-(pyridin-2-yl)propan-2-yl)-1H-pyrrolo[2,3-b]pyridine-6-carboxamide

Compound 304 was obtained (50 mg, 80%), as a yellow solid according to general procedure (VII) starting from Compound 303 (60 mg).

M/Z (M[³⁵Cl]+H)⁺: 409.8

### Compound 305: methyl 3-(4-chloro-3-fluorophenyl)-1-(2-(pyridin-2-yl)propan-2-yl)-1H-pyrrolo[2,3-b]pyridine-6-carboxylate

Compound 305 was prepared according to general procedure (VIII) starting from Compound 304 (50 mg). The residue was solubilized in EtOAc (100 mL), washed with water (100 mL), dried over magnesium sulfate and concentrated. The crude was purified by flash chromatography (Interchim® 20 μm, CyHex 100% to CyHex/EtOAc 80:20) to obtain Compound 305 (23 mg, 44%) as a white solid.

M/Z (M[³⁵Cl]+H)⁺: 424.8.

### Compound 306: Lithium 3-(4-chloro-3-fluorophenyl)-1-(2-(pyridin-2-yl)propan-2-yl)-1H-pyrrolo[2,3-b]pyridine-6-carboxylate

Compound 306 was prepared according to general procedure (IXb) starting from Compound 305 (23 mg) to obtain Compound 306 (22 mg, 99%) as a yellow solid.

M/Z (M[³⁵Cl]+H)⁺: 410.8

### Compound 307: methyl 6-(4-(3-(4-chloro-3-fluorophenyl)-1-(2-(pyridin-2-yl)propan-2-yl)-1H-pyrrolo[2,3-b]pyridine-6-carbonyl)-3,3-dimethylpiperazin-1-yl)-2,4-dimethylnicotinate

Compound 307 was prepared according to general procedure (Xa) starting from Compound 306 (61 mg) and Compound 95 (19 mg, 1.1 equiv), and using N,N-diisopropylethylamine (4.0 equiv). The crude was purified by flash chromatography (CyHex 100% to CyHex/EtOAc 20:80) to obtain Compound 307 (27 mg, 75%) as a yellow oil.

M/Z (M[³⁵Cl]+H)⁺: 669.3

### Example 131: 6-(4-(3-(4-chloro-3-fluorophenyl)-1-(2-(pyridin-2-yl)propan-2-yl)-1H-pyrrolo[2,3-b]pyridine-6-carbonyl)-3,3-dimethylpiperazin-1-yl)-2,4-dimethylnicotinic acid

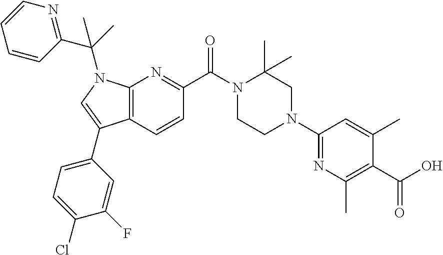 [SMILES: Cc1cc(N2CCN(C(=O)c3ccc4c(-c5ccc(Cl)c(F)c5)cn(C(C)(C)c5ccccn5)c4n3)C(C)(C)C2)nc(C)c1C(=O)O]

Example 131 was prepared according to general procedure (XIb) starting from Compound 307 (27 mg). The crude was purified by preparative HPLC (30% to 70% ACN/H₂O), then freeze-dried with water to obtain Example 131 (8 mg, 31%) as a white solid.

¹H-NMR (DMSO-d₆, 300 MHz) δ: 1.38 (s, 6H, 2 CH₃); 2.12 (s, 6H, 2 CH₃); 2.32 (s, 3H, Ar—CH₃); 2.41 (s, 3H, Ar—CH₃); 3.06-3.11 (m, 2H, CH₂); 3.22-3.26 (m, 2H, CH₂); 3.78 (s, 2H, CH₂); 6.33 (bs, 1H, Ar); 6.94 (d, J 7.9 Hz, 1H, Ar); 7.24 (ddd, J 7.3, 5.0, 0.8 Hz, 1H, Ar); 7.38 (d, J 8.4 Hz, 1H, Ar); 7.62-7.68 (m, 2H, Ar); 7.73 (dd, J 8.4, 1.8 Hz, 1H, Ar); 7.90 (ddd, J 11.2, 1.8 Hz, 1H, Ar); 8.45 (d, J 8.4 Hz, 1H, Ar); 8.47-8.49 (m, 1H, Ar); 8.50 (bs, 1H, Ar). 1 proton not observed.

M/Z (M[³⁵Cl]+H)⁺: 655.3.

### Compound 308: tert-butyl 3-(hydroxymethyl)-4-(5-(methoxycarbonyl)-4,6-dimethylpyridin-2-yl)piperazine-1-carboxylate

Compound 308 was prepared according to general procedure (XXI) starting from methyl 6-chloro-2,4-dimethylnicotinate (500 mg, 2.50 mmol) and tert-butyl 3-(hydroxymethyl)piperazine-1-carboxylate (596 mg, 1.1 equiv). The crude was purified by flash chromatography (CyHex 100% to CyHex/EtOAc 50:50) to obtain Compound 308 as a yellow gum (474 mg, 50%).

M/Z (M+H)⁺: 380.3

### Compound 309: tert-butyl 4-(5-(methoxycarbonyl)-4,6-dimethylpyridin-2-yl)-3-(methoxymethyl)piperazine-1-carboxylate

Compound 309 was prepared according to general procedure (XIII) in THE starting from Compound 308 (150 mg) and iodomethane (42 μL, 1.7 equiv). The crude was purified by flash chromatography (CyHex 100% to CyHex/EtOAc 50:50) to obtain Compound 309 (155 mg, 99%) as an off white solid.

M/Z (M+H)⁺: 394.3

### Compound 310: methyl 6-(2-(methoxymethyl)piperazin-1-yl)-2,4-dimethylnicotinate hydrochloride

Compound 310 was prepared according to general procedure (XVIIIc) starting from Compound 309 (155 mg). The reaction mixture was concentrated under reduced to obtain Compound 310 (125 mg, 96%) as a beige solid.

M/Z (M+H)⁺: 294.2.

### Compound 311: methyl (R)-6-(4-(1-(tert-butyl)-3-(4-chloro-3-fluorophenyl)-1H-pyrrolo[2,3-b]pyridine-6-carbonyl)-3-methylpiperazin-1-yl)-2,4-dimethylnicotinate

Compound 311 was prepared according to general procedure (Xb) starting from Compound 78 (67 mg) and Compound 310 (70 mg, 1.0 equiv), and using N,N-diisopropylethylamine (4.0 equiv). The crude was purified by flash chromatography (CyHex 100% to CyHex/EtOAc 50:50) to obtain Compound 311 (119 mg, 92%) as an off white solid.

M/Z (M[³⁵Cl]+H)⁺: 622.5

### Example 132: 6-(4-(1-(tert-butyl)-3-(4-chloro-3-fluorophenyl)-1H-pyrrolo[2,3-b]pyridine-6-carbonyl)-2-(methoxymethyl)piperazin-1-yl)-2,4-dimethyinicotinic acid

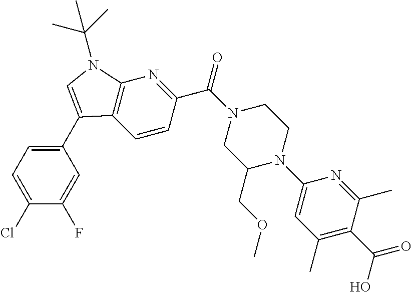 [SMILES: COCC1CN(C(=O)c2ccc3c(-c4ccc(Cl)c(F)c4)cn(C(C)(C)C)c3n2)CCN1c1cc(C)c(C(=O)O)c(C)n1]

Example 132 was prepared according to general procedure (XIb) starting from Compound 311 (119 mg). The crude was purified by flash chromatography (DCM 100% to DCM/MeOH 80:20) and freeze-dried from MeOH/water to obtain Example 132 (70 mg, 60%) as a white solid.

¹H-NMR (DMSO-d₆, 300 MHz) δ: 1.82 (s, 9H, C(CH₃)₃); 2.23 (s, 3H, CH₃); 2.34 (s, 3H, CH₃); 2.97 (s, 2H, CH₂); 3.13-3.22 (m, 2H, CH₂); 3.27 (s, 3H, CH₃); 3.51-3.68 (m, 1H, CH); 4.07-4.23 (m, 2H, CH₂); 4.34-4.53 (m, 1H, CH₂); 4.57-4.85 (m, 1H, CH₂); 6.44-6.51 (broad s, 1H, Ar); 7.48 (d, 1H, J 8.1 Hz, Ar); 7.60-7.68 (m, 2H, Ar); 7.83 (dd, 1H, J 10.5, 1.5 Hz, Ar); 8.23 (s 1H, Ar); 8.45 (d, 1H, J 8.1 Hz, Ar). 1 proton not observed.

(M[³⁵Cl]+H)⁺: 608.4.

### Compound 312: methyl 6-(4-(3-(4-chloro-3-fluorophenyl)-1-(2,2,2-trifluoroethyl)-1H-pyrrolo[2,3-b]pyridine-6-carbonyl)-3,3-dimethylpiperazin-1-yl)-2,4-dimethylnicotinate

Compound 312 was prepared according to general procedure (Xc) starting from Compound 83 (70 mg) and Compound 95 (65 mg, 1.1 equiv), and using N,N-diisopropylethylamine (4.0 equiv). The crude was purified by flash chromatography (CyHex 100% to CyHex/EtOAc 80:20) to obtain Compound 312 (83 mg, 70%) as a clear oil.

M/Z (M[³⁵Cl]+H)⁺: 632.2

### Example 133: 6-(4-(1-(carboxymethyl)-3-(4-chloro-3-fluorophenyl)-1H-pyrrolo[2,3-b]pyridine-6-carbonyl)-3,3-dimethylpiperazin-1-yl)-2,4-dimethylnicotinic acid

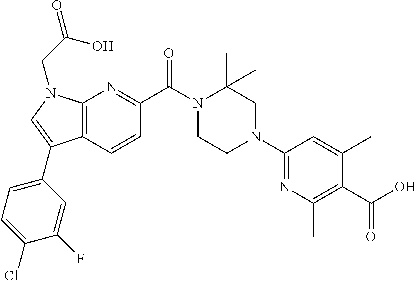 [SMILES: Cc1cc(N2CCN(C(=O)c3ccc4c(-c5ccc(Cl)c(F)c5)cn(CC(=O)O)c4n3)C(C)(C)C2)nc(C)c1C(=O)O]

Example 133 was prepared according to general procedure (XIb) starting from Compound 312 (83 mg). The crude was purified by preparative HPLC (20% to 60% ACN/H₂O), then freeze-dried with water to obtain Example 133 (20 mg, 26%) as a white solid.

¹H-NMR (DMSO-d₆, 300 MHz) δ: 1.53 (s, 6H, 2 CH₃); 2.28 (s, 3H, Ar—CH₃); 2.39 (s, 3H, Ar—CH₃); 3.50-3.54 (m, 2H, CH₂); 3.75-3.78 (m, 2H, CH₂); 3.93 (s, 2H, CH₂); 5.09 (s, 2H, HOOC—CH₂); 6.34 (s, 1H, Ar); 7.44 (d, J 8.2 Hz, 1H, Ar); 7.59-7.68 (m, 2H, Ar); 7.73-7.77 (m, 1H, Ar); 8.22 (s, 1H, Ar); 8.48 (d, J 8.2 Hz, 1H, Ar). 2 protons not observed.

M/Z (M[³⁵Cl]+H)⁺: 593.9.

### Compound 313: 6-chloro-3-(4-chloro-3-fluorophenyl)-1-(2-methoxyethyl)-1H-pyrrolo[2,3-b]pyridine

Compound 313 was prepared according to general procedure (IV) starting from Compound 3 (450 mg) and 2-methoxyethan-1-amine (265 mg, 2.5 equiv). The crude was purified by flash chromatography (CyHex 100% to CyHex/EtOAc 90:10) to obtain Compound 313 (258 mg, 48%) as a yellow oil.

M/Z (M[³⁵Cl]+H)⁺: 339.1

### Compound 314: 3-(4-chloro-4-fluorophenyl)-1-(2-methoxyethyl)-1H-pyrrolo[2,3-b]pyridine-6-carbonitrile

Compound 314 was prepared according to general procedure (Va) starting from Compound 313 (258 mg). The crude was purified by flash chromatography (CyHex 100% to CyHex/EtOAc 80:20) to obtain Compound 314 (198 mg, 79%) as a yellow solid.

M/Z (M[³⁵Cl]+H)⁺: 330.1

### Compound 315: 3-(4-chloro-3-fluorophenyl)-1-(2-methoxyethyl)-1H-pyrrolo[2,3-b]pyridine-6-carboxamide

Compound 315 was obtained (238 mg), as a yellow wax according to general procedure (VII) starting from Compound 314 (198 mg).

M/Z (M[³⁵Cl]+H)⁺: 348.2

### Compound 316: methyl 3-(4-chloro-3-fluorophenyl)-1-(2-methoxyethyl)-1H-pyrrolo[2,3-b]pyridine-6-carboxylate

Compound 316 was prepared according to general procedure (VIII) starting from Compound 315 (600 μmol). The crude was purified by flash chromatography (CyHex 100% to CyHex/EtOAc 50:50) to obtain Compound 316 (188 mg, 86% over two steps) as an off white solid.

M/Z (M[³⁵Cl]+H)⁺: 363.1.

### Compound 317: 3-(4-chloro-3-fluorophenyl)-1-(2-methoxyethyl)-1H-pyrrolo[2,3-b]pyridine-6-carboxylic acid

Compound 317 was prepared according to general procedure (IXa) starting from Compound 316 (188 mg) to obtain Compound 317 (180 mg, 99%) as a yellow solid.

M/Z (M[³⁵Cl]+H)⁺: 349.1

### Compound 318: methyl 6-(4-(3-(4-chloro-3-fluorophenyl)-1-(2-methoxyethyl)-1H-pyrrolo[2,3-b]pyridine-6-carbonyl)-3,3-dimethylpiperazin-1-yl)-2,4-dimethylnicotinate

Compound 318 was prepared according to general procedure (Xa) starting from Compound 317 (180 mg) and Compound 95 (179 mg, 1.1 equiv), and using N,N-diisopropylethylamine (4.0 equiv). The crude was purified by flash chromatography (CyHex 100% to CyHex/EtOAc 70:30) to obtain Compound 318 (274 mg, 87%) as a white solid.

M/Z (M[³⁵Cl]+H)⁺: 608.4

### Example 134: 6-(4-(3-(4-chloro-3-fluorophenyl)-1-(2-methoxyethyl)-1H-pyrrolo[2,3-b]pyridine-6-carbonyl)-3,3-dimethylpiperazin-1-yl)-2,4-dimethyinicotinic acid

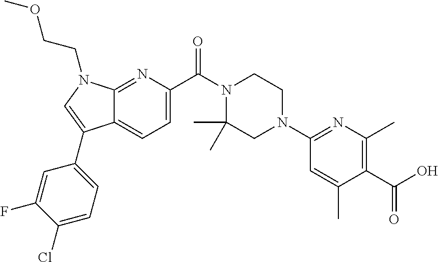 [SMILES: COCCn1cc(-c2ccc(Cl)c(F)c2)c2ccc(C(=O)N3CCN(c4cc(C)c(C(=O)O)c(C)n4)CC3(C)C)nc21]

Example 134 was prepared according to general procedure (XIb) starting from Compound 318 (274 mg). The crude was purified by flash chromatography (DCM 100% to DCM/MeOH 80:20) then freeze-dried with EtOH/water to obtain Example 134 (207 mg, 77%) as a beige solid.

¹H-NMR (DMSO-d₆, 300 MHz) δ: 1.54 (s, 6H, 2*CH₃); 2.26 (s, 3H, CH₃); 2.38 (s, 3H, CH₃); 3.72 (s, 3H, OCH₃); 3.55 (t, J 5.1 Hz, 2H, CH₂); 3.77-3.80 (m, 4H, 2*CH₂); 3.92 (s, 2H, CH₂N); 4.49 (t, J 5.1 Hz, 2H, CH₂); 6.32 (s, 1H, Ar); 7.40 (d, 1H, J 8.4 Hz, Ar); 7.60-7.67 (m, 2H, Ar); 7.76 (dd, 1H, J 11.7, 1.8 Hz, Ar); 8.24 (s, 1H, Ar); 8.46 (d, 1H, J 8.4 Hz, Ar). 1 proton not observed.

M/Z (M[³⁵Cl]+H)⁺: 594.5.

### Compound 319: 6-chloro-3-(4-chloro-3-fluorophenyl)-1-(pyridin-3-ylmethyl)-1H-pyrrolo[2,3-b]pyridine

Compound 319 was prepared according to general procedure (IV) starting from Compound 3 (450 mg) and pyridin-3-ylmethanamine (382 mg, 2.5 equiv). The crude was purified by flash chromatography (CyHex 100% to CyHex/EtOAc 70:30) to obtain Compound 319 (310 mg, 53%) as a yellow solid.

M/Z (M[³⁵Cl]+H)⁺: 372.2

### Compound 320: 3-(4-chloro-3-fluorophenyl)-1-(pyridin-3-ylmethyl)-1H-pyrrolo[2,3-b]pyridine-6-carbonitrile

Compound 320 was prepared according to general procedure (Va) starting from Compound 319 (310 mg). The crude was purified by flash chromatography (CyHex 100% to CyHex/EtOAc 50:50) to obtain Compound 320 (260 mg) as a yellow solid.

M/Z (M[³⁵Cl]+H)⁺: 363.1.

### Compound 321: 3-(4-chloro-3-fluorophenyl)-1-(pyridin-3-ylmethyl)-1H-pyrrolo[2,3-b]pyridine-6-carboxamide

Compound 321 was prepared according to general procedure (VII) starting from Compound 320 (260 mg). The crude was purified by flash chromatography (CyHex 100% to EtOAc 100% then to EtOAc/MeOH 90:10) to obtain Compound 321 (310 mg) as a yellow solid.

M/Z (M[³⁵Cl]+H)⁺: 381.2

### Compound 322: methyl 3-(4-chloro-3-fluorophenyl)-1-(pyridin-3-ylmethyl)-1H-pyrrolo[2,3-b]pyridine-6-carboxylate

Compound 322 was prepared according to general procedure (VIII) starting from Compound 321 (250 mg). DMA (5 mL), DMF-DMA (870 μL, 10 eq) and sodium methoxide (106 mg, 3 eq) were added, and the reaction mixture was heated at 75° C. for another 18 hours, then concentrated under reduced pressure. The residue taken up in water (150 mL) and extracted with EtOAc (2*100 mL), dried over magnesium sulfate and concentrated. The crude was purified by flash chromatography (CyHex 100% to EtOAc 100%) to obtain Compound 322 (130 mg) as a white solid.

M/Z (M[³⁵Cl]+H)⁺: 396.2

### Compound 323: 3-(4-chloro-3-fluorophenyl)-1-(pyridin-3-ylmethyl)-1H-pyrrolo[2,3-b]pyridine-6-carboxylic acid

Compound 323 was prepared according to general procedure (IXa) starting from Compound 322 (23 mg) to obtain Compound 323 (60 mg, 22% over three steps) as a yellow solid.

M/Z (M[³⁵Cl]+H)⁺: 382.2

### Compound 324: methyl 6-(4-(3-(4-chloro-3-fluorophenyl)-1-(pyridin-3-ylmethyl)-1H-pyrrolo[2,3-b]pyridine-6-carbonyl)-3,3-dimethylpiperazin-1-yl)-2,4-dimethylnicotinate

Compound 324 was prepared according to general procedure (Xa) starting from Compound 323 (58 mg) and Compound 95 (52 mg, 1.1 equiv), and using N,N-diisopropylethylamine (4.0 equiv). The crude was purified by flash chromatography (CyHex 100% to EtOAc 100%) to obtain Compound 324 (94 mg) as a yellow oil.

M/Z (M[³⁵Cl]+H)⁺: 641.4

### Example 135: 6-(4-(3-(4-chloro-3-fluorophenyl)-1-(pyridin-3-ylmethyl)-1H-pyrrolo[2,3-b]pyridine-6-carbonyl)-3,3-dimethylpiperazin-1-yl)-2,4-dimethylnicotinic acid hydrochloride

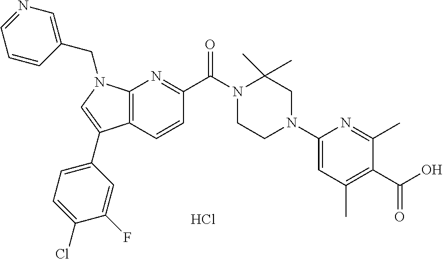 [SMILES: Cc1cc(N2CCN(C(=O)c3ccc4c(-c5ccc(Cl)c(F)c5)cn(Cc5cccnc5)c4n3)C(C)(C)C2)nc(C)c1C(=O)O ; Cl]

Example 135 was prepared according to general procedure (XIb) starting from Compound 324 (90 mg). The crude was purified by preparative HPLC (Column B, 5% to 100% ACN/H₂O), then freeze-dried with water to obtain Example 135 (43 mg, 46%) as a white solid.

¹H-NMR (DMSO-d₆ 300 MHz) δ: 1.53 (s, 6H, (CH₃)₂); 2.30 (s, 3H, CH₃); 2.42 (s, 3H, CH₃); 3.40-3.46 (m, 2H, CH₂); 3.69-3.73 (m, 2H, CH₂); 3.93 (s, 2H, CH₂); 5.68 (s, 2H, CH₂); 6.46 (bs, 1H, Ar); 7.44 (d, J 8.4 Hz, 1H, Ar); 7.61-7.68 (m, 3H, Ar); 7.77 (d, J 9.9 Hz, 1H, Ar); 8.00-8.07 (m, 1H, Ar); 8.40 (s, 1H, Ar); 8.50 (d, J 8.4 Hz, 1H, Ar); 8.65-8.69 (m, 1H, Ar); 8.80 (bs, 1H, Ar). 2 protons not observed.

M/Z (M[³⁵Cl]+H)⁺: 627.3

### Compound 325: (S)-6-chloro-3-(4-chloro-3-fluorophenyl)-1-(1-methoxypropan-2-yl)-1H-pyrrolo[2,3-b]pyridine

Compound 325 was prepared according to general procedure (IV) starting from Compound 3 (450 mg) and (S)-1-methoxypropan-2-amine (315 mg, 2.5 equiv). The crude was purified by flash chromatography (CyHex 100% to CyHex/EtOAc 90:10) to obtain Compound 325 (285 mg, 51%) as a yellow oil.

M/Z (M[³⁵Cl]+H)⁺: 353.7

### Compound 326: (S)-3-(4-chloro-3-fluorophenyl)-1-(1-methoxypropan-2-yl)-1H-pyrrolo[2,3-b]pyridine-6-carbonitrile

Compound 326 was prepared according to general procedure (Va) starting from Compound 325 (285 mg). The crude was purified by flash chromatography (CyHex 100% to CyHex/EtOAc 90:10) to obtain Compound 326 (180 mg, 65%) as a yellow solid.

M/Z (M[³⁵Cl]+H)⁺: 344.6

### Compound 327: (S)-3-(4-chloro-3-fluorophenyl)-1-(1-methoxypropan-2-yl)-1H-pyrrolo[2,3-b]pyridine-6-carboxamide

Compound 327 was obtained (178 mg, 94%), as a yellow solid according to general procedure (VII) starting from Compound 326 (180 mg).

M/Z (M[³⁵Cl]+H)⁺: 362.6

### Compound 328: methyl (S)-3-(4-chloro-3-fluorophenyl)-1-(1-methoxypropan-2-yl)-1H-pyrrolo[2,3-b]pyridine-6-carboxylate

Compound 328 prepared according to general procedure (VIII) starting from Compound 327 (178 mg). The crude was purified by flash chromatography (CyHex 100% to CyHex/EtOAc 80:20) to obtain Compound 328 (172 mg, 93%) as a yellow oil.

M/Z (M[³⁵Cl]+H)⁺: 377.8

### Compound 329: (S)-3-(4-chloro-3-fluorophenyl)-1-(1-methoxypropan-2-yl)-1H-pyrrolo[2,3-b]pyridine-6-carboxylic acid

Compound 329 was prepared according to general procedure (IXa) starting from Compound 328 (172 mg) to obtain Compound 329 (157 mg, 95%) as a beige solid.

M/Z (M[³⁵Cl]+H)⁺: 363.7

### Compound 330: methyl (S)-6-(4-(3-(4-chloro-3-fluorophenyl)-1-(1-methoxypropan-2-yl)-1H-pyrrolo[2,3-b]pyridine-6-carbonyl)-3,3-dimethylpiperazin-1-yl)-2,4-dimethylnicotinate

Compound 330 was prepared according to general procedure (Xc) starting from Compound 329 (70 mg) and Compound 95 (67 mg, 1.1 equiv), and using N,N-diisopropylethylamine (4.0 equiv). The crude was purified by flash chromatography (Interchim® 20 μm, CyHex 100% to CyHex/EtOAc 70:30) to obtain Compound 330 (125 mg) as a clear oil.

M/Z (M[³⁵Cl]+H)⁺: 622.4

### Example 136: (S)-6-(4-(3-(4-chloro-3-fluorophenyl)-1-(1-methoxypropan-2-yl)-1H-pyrrolo[2,3-b]pyridine-6-carbonyl)-3,3-dimethylpiperazin-1-yl)-2,4-dimethylnicotinic acid

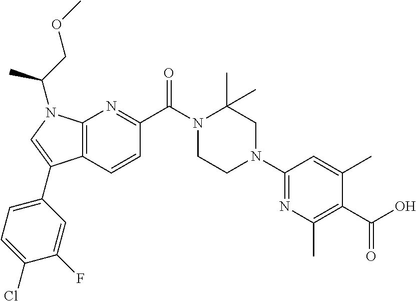 [SMILES: COC[C@H](C)n1cc(-c2ccc(Cl)c(F)c2)c2ccc(C(=O)N3CCN(c4cc(C)c(C(=O)O)c(C)n4)CC3(C)C)nc21]

Example 136 was prepared according to general procedure (XIb) starting from Compound 330 (125 mg). The crude was triturated in ACN (2*2 mL), in Et2O (2 mL), then dissolved in DCM and concentrated under reduced pressure. The residue was freeze-dried twice with EtOH/water (1:9, 150 mL) to obtain Example 136 (50 mg, 43%) as a yellow solid.

¹H-NMR (DMSO-d₆, 300 MHz) δ: 1.52-1.54 (m, 9H, 2 CH₃+(S) CH₃); 2.27 (s, 3H, Ar—CH₃); 2.38 (s, 3H, Ar—CH₃); 3.24 (m, 3H, O—CH₃); 3.49-3.58 (m, 2H, CH₂); 3.65-3.70 (m, 1H, CHₐ); 3.76-3.86 (m, 3H, CH_{b}+CH₂); 3.93 (s, 2H, CH₂); 5.15-5.27 (m, 1H, CH—CH₃); 6.33 (s, 1H, Ar); 7.40 (d, J 8.2 Hz, 1H, Ar); 7.61-7.68 (m, 2H, Ar); 7.78-7.82 (m, 1H, Ar); 8.37 (s, 1H, Ar); 8.45 (d, J 8.2 Hz, 1H, Ar). 1 proton not observed.

M/Z (M[³⁵Cl]+H)⁺: 608.4.

Mp: 203-207° C.

### Compound 331: tert-butyl 4-(5-(ethoxycarbonyl)pyridin-2-yl)-2,2-dimethylpiperazine-1-carboxylate

Compound 331 was prepared according to general procedure (XXI) starting from ethyl 6-chloronicotinate (500 mg) and tert-butyl 2,2-dimethylpiperazine-1-carboxylate (693 mg). The crude residue was purified by flash chromatography (CyHex 100% to CyHex/EtOAc 30:70) to obtain Compound 331 (578 mg, 59%) as a white solid.

M/Z (M+H)⁺: 364.3

### Compound 332: ethyl 6-(3,3-dimethylpiperazin-1-yl)nicotinate hydrochloride

Compound 332 was obtained (613 mg, n.d.) as a yellow solid according to general procedure (XVIIIa) starting from Compound 331 (578 mg).

M/Z (M+H)⁺: 264.2

### Compound 333: methyl 6-(4-(1-(tert-butyl)-3-(4-chloro-3-fluorophenyl)-1H-pyrrolo[2,3-b]pyridine-6-carbonyl)-3,3-dimethylpiperazin-1-yl)nicotinate

Compound 333 was prepared according to general procedure (Xc) starting from Compound 78 (240 mg) and Compound 332 (229 mg, 1.1 equiv), and using N,N-diisopropylethylamine (4.0 equiv). The crude was purified by flash chromatography (CyHex 100% to CyHex/EtOAc 80:20) to obtain Compound 333 (376 mg, 92%) as a yellow oil.

M/Z (M[³⁵Cl]+H)⁺: 592.3

### Example 137: 6-(4-(1-(tert-butyl)-3-(4-chloro-3-fluorophenyl)-1H-pyrrolo[2,3-b]pyridine-6-carbonyl)-3,3-dimethylpiperazin-1-yl)nicotinic acid

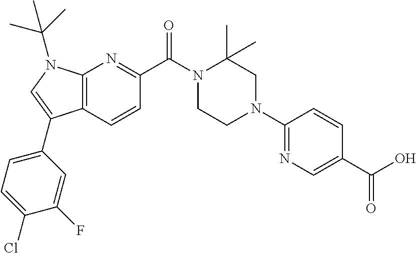 [SMILES: CC1(C)CN(c2ccc(C(=O)O)cn2)CCN1C(=O)c1ccc2c(-c3ccc(Cl)c(F)c3)cn(C(C)(C)C)c2n1]

Example 137 was prepared according to general procedure (IXa) starting from Compound 333 (376 mg) to obtain Example 137 (320 mg, 89%) as a yellow solid.

¹H-NMR (DMSO-d₆, 300 MHz) δ: 1.56 (s, 6H, 2 CH₃); 1.82 (s, 9H, tBu); 3.56-3.64 (m, 2H, CH₂); 3.87-3.94 (m, 2H, CH₂); 4.02 (s, 2H, CH₂); 6.66 (d, J 9.0 Hz, 1H, Ar); 7.41 (d, J 8.3 Hz, 1H, Ar); 7.60-7.68 (m, 2H, Ar); 7.80-7.84 (m, 1H, Ar); 7.97 (dd, J 9.1, 2.3 Hz, 1H, Ar); 8.21 (s, 1H, Ar); 8.43 (d, J 8.3 Hz, 1H, Ar); 8.64 (d, J 2.3 Hz, 1H, Ar). 1 proton not observed.

M/Z (M[³⁵Cl]+H)⁺: 564.4.

### Example 138: 6-(4-(1-(tert-butyl)-3-(4-chloro-3-fluorophenyl)-1H-pyrrolo[2,3-b]pyridine-6-carbonyl)-3,3-dimethylpiperazin-1-yl)nicotinamide

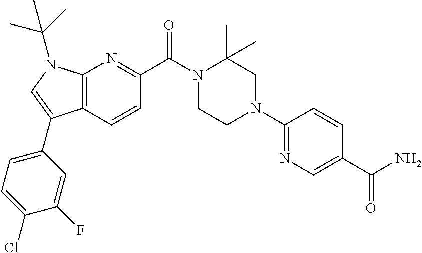 [SMILES: CC1(C)CN(c2ccc(C(N)=O)cn2)CCN1C(=O)c1ccc2c(-c3ccc(Cl)c(F)c3)cn(C(C)(C)C)c2n1]

To a solution of Example 137 (70 mg) in ammonia 0.5M in dioxane (1.2 mL, 0.5 molar, 5 Eq) was added ((1H-benzo[d][1,2,3]triazol-1-yl)oxy)tris(dimethylamino)phosphonium hexafluorophosphate(V) (71 mg, 1.3 Eq). The reaction was stirred at 25° C. for 2 hour. The reaction mixture was quenched with NH4Cl sat. (40 mL) then extracted with EtOAc (2*40 mL). The organic layer was dried over MgSO₄ then concentrated to dryness. The crude was purified by preparative HPLC (40% to 80% ACN/H₂O), freeze-dried with water, then freeze-dried with water+HCl 1N in water (5 eq) (action repeated 2 times) to obtain Example 138 (18 mg, 24%) as a white solid.

¹H-NMR (DMSO-d₆, 300 MHz) δ: 1.59 (s, 6H, 2 CH₃); 1.82 (s, 9H, tBu); 3.67-3.70 (m, 2H, CH₂); 3.93-3.97 (m, 2H, CH₂); 4.08 (s, 2H, CH₂); 6.99-7.09 (m, 1H, Ar); 7.32-7.40 (m, 1H, NHₐH_{b}); 7.42 (d, J 8.3 Hz, 1H, Ar); 7.59-7.68 (m, 2H, Ar); 7.80-7.84 (m, 1H, Ar); 7.97-8.12 (m, 1H, NHₐH_{b}); 8.20-8.24 (m, 2H, Ar); 8.44 (d, J 8.3 Hz, 1H, Ar); 8.57-8.58 (m, 1H, Ar)

M/Z (M[³⁵Cl]+H)⁺: 563.4.

### Example 139: 6-(4-(1-(tert-butyl)-3-(4-chloro-3-fluorophenyl)-1H-pyrrolo[2,3-b]pyridine-6-carbonyl)-3,3-dimethylpiperazin-1-yl)-N,N-dimethylnicotinamide

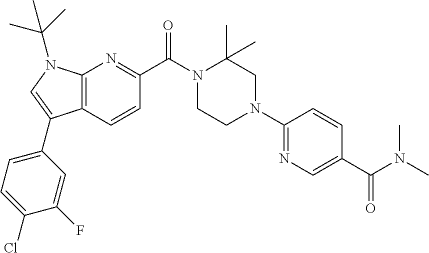 [SMILES: CN(C)C(=O)c1ccc(N2CCN(C(=O)c3ccc4c(-c5ccc(Cl)c(F)c5)cn(C(C)(C)C)c4n3)C(C)(C)C2)nc1]

Example 139 was prepared according to general procedure (Xa) starting from Example 137 (70 mg) and dimethylamine in THF (2M, 1.1 eq) and using N,N-diisopropylethylamine (4.0 equiv). The crude was purified by preparative HPLC (40% to 80% ACN/H₂O), freeze-dried with water, then freeze-dried with water+HCl 1N in water (5 eq) (action repeated 2 times) to obtain Example 139 (26 mg, 33%) as a white solid.

¹H-NMR (DMSO-d₆, 300 MHz) δ: 1.59 (s, 6H, 2 CH₃); 1.82 (s, 9H, tBu); 2.99 (s, 6H, N—(CH₃)₂); 3.60-3.66 (m, 2H, CH₂); 3.90-3.96 (m, 2H, CH₂); 4.01 (s, 2H, CH₂); 6.88-6.91 (m, 1H, Ar); 7.42 (d, J 8.3 Hz, 1H, Ar); 7.60-7.69 (m, 2H, Ar); 7.78-7.85 (m, 2H, Ar); 8.18-8.22 (m, 2H, Ar); 8.44 (d, J 8.3 Hz, 1H, Ar).

M/Z (M[³⁵Cl]+H)⁺: 591.4

### Compound 334: (R)-6-chloro-3-(4-chloro-3-fluorophenyl)-1-(1-methoxypropan-2-yl)-1H-pyrrolo[2,3-b]pyridine

Compound 334 was prepared according to general procedure (IV) starting from Compound 3 (500 mg) and (R)-1-methoxypropan-2-amine (350 mg, 2.5 equiv). The crude was purified by flash chromatography (CyHex 100% to CyHex/EtOAc 90:10) to obtain Compound 334 (320 mg, 58%) as a yellow oil.

M/Z (M[³⁵Cl]+H)⁺: 353.7

### Compound 335: (R)-3-(4-chloro-3-fluorophenyl)-1-(1-methoxypropan-2-yl)-1H-pyrrolo[2,3-b]pyridine-6-carbonitrile

Compound 335 was prepared according to general procedure (Va) starting from Compound 334 (320 mg). The crude was purified by flash chromatography (CyHex 100% to CyHex/EtOAc 90:10) to obtain Compound 335 (165 mg, 53%) as a yellow solid.

M/Z (M[³⁵Cl]+H)⁺: 344.7

### Compound 336: (R)-3-(4-chloro-3-fluorophenyl)-1-(1-methoxypropan-2-yl)-1H-pyrrolo[2,3-b]pyridine-6-carboxamide

Compound 336 was obtained (144 mg, 83%), as a yellow solid according to general procedure (VII) starting from Compound 335 (165 mg).

M/Z (M[³⁵Cl]+H)⁺: 362.7

### Compound 337: methyl (R)-3-(4-chloro-3-fluorophenyl)-1-(1-methoxypropan-2-yl)-1H-pyrrolo[2,3-b]pyridine-6-carboxylate

Compound 337 prepared according to general procedure (VIII) starting from Compound 336 (144 mg). The crude was purified by flash chromatography (CyHex 100% to CyHex/EtOAc 80:20) to obtain Compound 337 (139 mg, 93%) as a yellow oil.

M/Z (M[³⁵Cl]+H)⁺: 377.7

### Compound 338: (R)-3-(4-chloro-3-fluorophenyl)-1-(1-methoxypropan-2-yl)-1H-pyrrolo[2,3-b]pyridine-6-carboxylic acid

Compound 338 was prepared according to general procedure (IXa) starting from Compound 337 (140 mg) to obtain Compound 338 (122 mg, 91%) as a yellow solid.

M/Z (M[³⁵Cl]+H)⁺: 363.7

### Compound 339: methyl (R)-6-(4-(3-(4-chloro-3-fluorophenyl)-1-(1-methoxypropan-2-yl)-1H-pyrrolo[2,3-b]pyridine-6-carbonyl)-3,3-dimethylpiperazin-1-yl)-2,4-dimethylnicotinate

Compound 339 was prepared according to general procedure (Xc) starting from Compound 338 (122 mg) and Compound 95 (116 mg, 1.1 equiv), and using N,N-diisopropylethylamine (4.0 equiv). The crude was purified by flash chromatography (CyHex 100% to CyHex/EtOAc 70:30) to obtain Compound 339 (228 mg) as a yellow oil.

M/Z (M[³⁵Cl]+H)⁺: 622.5

### Example 140: (R)-6-(4-(3-(4-chloro-3-fluorophenyl)-1-(1-methoxypropan-2-yl)-1H-pyrrolo[2,3-b]pyridine-6-carbonyl)-3,3-dimethylpiperazin-1-yl)-2,4-dimethyinicotinic acid

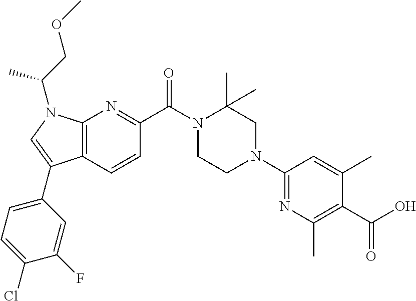 [SMILES: COC[C@@H](C)n1cc(-c2ccc(Cl)c(F)c2)c2ccc(C(=O)N3CCN(c4cc(C)c(C(=O)O)c(C)n4)CC3(C)C)nc21]

Example 140 was prepared according to general procedure (XIb) starting from Compound 339 (336 μmol). The crude was triturated in ACN (2*2 mL), in Et₂O (2 mL), then dissolved in DCM and concentrated under reduced pressure. The residue was freeze-dried twice with EtOH/water (1:9, 150 mL) to obtain Example 140 (97 mg, 47% over two steps) as a white solid.

¹H-NMR (DMSO-d₆, 300 MHz) δ: 1.52-1.54 (m, 9H, 2 CH₃+(R) CH₃); 2.27 (s, 3H, Ar—CH₃); 2.39 (s, 3H, Ar—CH₃); 3.24 (m, 3H, O—CH₃); 3.49-3.58 (m, 2H, CH₂); 3.65-3.70 (m, 1H, CHₐ); 3.77-3.87 (m, 3H, CH_{b}+CH₂); 3.92-3.97 (m, 2H, CH₂); 5.15-5.27 (m, 1H, CH—CH₃); 6.33 (s, 1H, Ar); 7.40 (d, J 8.2 Hz, 1H, Ar); 7.61-7.68 (m, 2H, Ar); 7.78-7.82 (m, 1H, Ar); 8.37 (s, 1H, Ar); 8.45 (d, J 8.2 Hz, 1H, Ar); 12.67 (s, 1H, COOH).

M/Z (M[³⁵Cl]+H)⁺: 608.2.

### Compound 340: methyl 6-(4-(3-bromo-1-isobutyl-1H-pyrrolo[2,3-b]pyridine-6-carbonyl)-3,3-dimethylpiperazin-1-yl)-2,4-dimethylnicotinate

Compound 340 was prepared according to general procedure (Xa) starting from Compound 98 (0.92 g) and Compound 95 (1.07 g, 1.1 equiv), and using N,N-diisopropylethylamine (4.0 equiv). The crude was purified by flash chromatography (CyHex 100% to CyHex/EtOAc 50:50) to obtain Compound 340 (1.60 g, 93%) as an off white solid.

M/Z (M[⁷⁹Br]+H)⁺: 558.2

### Compound 341: methyl 6-(4-(3-(3,4-difluorophenyl)-1-isobutyl-1H-pyrrolo[2,3-b]pyridine-6-carbonyl)-3,3-dimethylpiperazin-1-yl)-2,4-dimethylnicotinate

Compound 341 was prepared according to general procedure (XVIIa) starting from Compound 340 (200 mg) and (3,4-difluorophenyl)boronic acid (68 mg, 1.2 equiv). The crude was purified by flash chromatography (CyHex 100% to CyHex/EtOAc 80:20) then by preparative HPLC (Column B, 5% to 100% ACN/H₂O) and freeze-dried with water, to obtain Compound 341 (115 mg, 54%) as a white solid.

M/Z (M+H)⁺: 590.5

### Example 141: 6-(4-(3-(3,4-difluorophenyl)-1-isobutyl-1H-pyrrolo[2,3-b]pyridine-6-carbonyl)-3,3-dimethylpiperazin-1-yl)-2,4-dimethylnicotinic acid

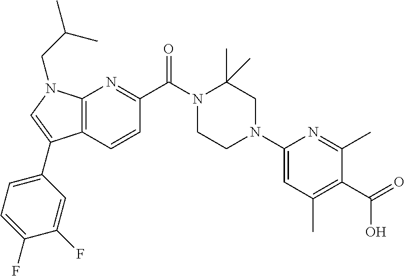 [SMILES: Cc1cc(N2CCN(C(=O)c3ccc4c(-c5ccc(F)c(F)c5)cn(CC(C)C)c4n3)C(C)(C)C2)nc(C)c1C(=O)O]

Example 141 was prepared according to general procedure (XIb) starting from Compound 341 (115 mg). The crude was purified by flash chromatography (DCM 100% to DCM/MeOH 80:20) and freeze-dried with EtOH/water to obtain Example 141 (76 mg, 68%) as a white solid.

¹H-NMR (DMSO-d₆, 300 MHz) δ:0.92 (d, J 6.6 Hz, 6H, 2*CH₃); 1.53 (s, 6H, 2*CH₃); 2.21-2.29 (m, 4H, CH, CH₃); 2.37 (s, 3H, CH₃); 3.54-3.61 (m, 2H, CH₂); 3.78-3.84 (m, 2H, CH₂); 3.90 (s, 2H, CH₂); 4.14 (d, 2H, J 7.5 Hz, CH₂); 6.31 (s, 1H, Ar); 7.42 (d, 1H, J 8.4 Hz, Ar); 7.46-7.55 (m, 1H, Ar); 7.56-7.61 (m 1H, Ar); 7.74-7.81 (m 1H, Ar); 8.19 (s, 1H, Ar); 8.43 (d, 1H, J 8.4 Hz, Ar). 1 proton not observed.

M/Z (M+H)⁺: 576.4.

### Compound 342: methyl 6-(4-(3-(3-fluoro-4-methylphenyl)-1-isobutyl-1H-pyrrolo[2,3-b]pyridine-6-carbonyl)-3,3-dimethylpiperazin-1-yl)-2,4-dimethylnicotinate

Compound 342 was prepared according to general procedure (XVIIa) starting from Compound 340 (200 mg) and (3-fluoro-4-methylphenyl)boronic acid (66 mg, 1.2 equiv). The crude was purified by flash chromatography (CyHex 100% to CyHex/EtOAc 55:45) then by flash chromatography (Interchim® 20 μm, CyHex 100% to CyHex/EtOAc 65:35) to obtain Compound 342 (92 mg, 44%) as a white solid.

M/Z (M+H)⁺: 586.5

### Example 142: 6-(4-(3-(3-fluoro-4-methylphenyl)-1-isobutyl-1H-pyrrolo[2,3-b]pyridine-6-carbonyl)-3,3-dimethylpiperazin-1-yl)-2,4-dimethylnicotinic acid

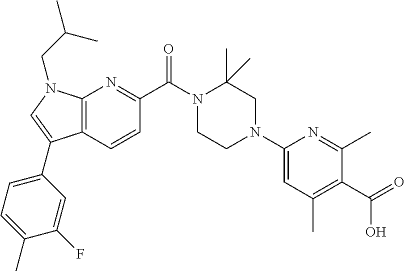 [SMILES: Cc1ccc(-c2cn(CC(C)C)c3nc(C(=O)N4CCN(c5cc(C)c(C(=O)O)c(C)n5)CC4(C)C)ccc23)cc1F]

Example 142 was prepared according to general procedure (XIb) starting from Compound 342 (92 mg). The crude was purified by flash chromatography (DCM 100% to DCM/MeOH 80:20) and freeze-dried with EtOH/water to obtain Example 142 (70 mg, 78%) as a beige solid.

¹H-NMR (DMSO-d₆, 300 MHz) δ:0.91 (d, J 6.6 Hz, 6H, 2*CH₃); 1.53 (s, 6H, 2*CH₃); 2.21-2.29 (m, 7H, CH, 2*CH₃); 2.36 (s, 3H, CH₃); 3.54-3.62 (m, 2H, CH₂); 3.76-3.84 (m, 2H, CH₂); 3.90 (s, 2H, CH₂); 4.14 (d, 2H, J 7.2, CH₂); 6.29 (s, 1H, Ar); 7.32-7.42 (d, 2H, Ar); 7.47-7.51 (m, 2H, Ar); 8.17 (s, 1H, Ar); 8.42 (d, 1H, J 8.1 Hz, Ar). 1 proton not observed.

M/Z (M+H)⁺: 572.5.

### Compound 343: methyl 6-(4-(1-isobutyl-3-(3,4,5-trifluorophenyl)-1H-pyrrolo[2,3-b]pyridine-6-carbonyl)-3,3-dimethylpiperazin-1-yl)-2,4-dimethylnicotinate

Compound 343 was prepared according to general procedure (XVIIa) starting from Compound 340 (200 mg) and (3,4,5-trifluorophenyl)boronic acid (95 mg, 1.5 equiv). The crude was purified by flash chromatography (CyHex 100% to CyHex/EtOAc 60:40) to obtain Compound 343 (175 mg, 80%) as a white solid.

M/Z (M+H)⁺: 608.4

### Example 143: 6-(4-(1-isobutyl-3-(3,4,5-trifluorophenyl)-1H-pyrrolo[2,3-b]pyridine-6-carbonyl)-3,3-dimethylpiperazin-1-yl)-2,4-dimethylnicotinic acid

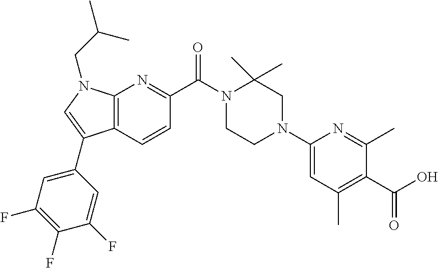 [SMILES: Cc1cc(N2CCN(C(=O)c3ccc4c(-c5cc(F)c(F)c(F)c5)cn(CC(C)C)c4n3)C(C)(C)C2)nc(C)c1C(=O)O]

Example 143 was prepared according to general procedure (XIb) starting from Compound 343 (175 mg). The crude was purified by flash chromatography (DCM 100% to DCM/MeOH 80:20), triturated in Et₂O and freeze-dried with EtOH/water to obtain Example 143 (30 mg, 18%) as a white solid.

¹H-NMR (DMSO-d₆, 300 MHz) δ: 0.91 (d, J 7.0 Hz, 6H, (CH₃)₂); 1.53 (s, 6H, (CH₃)₂); 2.24-2.27 (m, 4H, CH₃, CH); 2.37 (s, 3H, CH₃); 3.55-3.59 (m, 2H, CH₂); 3.79-3.82 (m, 2H, CH₂); 3.91 (s, 2H, CH₂); 4.13 (d, J 7.0 Hz, 2H, CH₂); 6.33 (s, 1H, Ar); 7.42 (d, J 8.2 Hz, 1H, Ar); 7.65-7.70 (m, 2H, Ar); 8.28 (s, 1H, Ar); 8.50 (d, J 8.2 Hz, 1H, Ar); 12.58 (bs, 1H, COOH).

M/Z (M+H)⁺: 594.5

### Compound 344: methyl 6-(4-(3-(3-fluoro-4-(trifluoromethyl)phenyl)-1-isobutyl-1H-pyrrolo[2,3-b]pyridine-6-carbonyl)-3,3-dimethylpiperazin-1-yl)-2,4-dimethylnicotinate

Compound 344 was prepared according to general procedure (XVIIa) starting from Compound 340 (200 mg) and 2-(3-fluoro-4-(trifluoromethyl)phenyl)-4,4,5,5-tetramethyl-1,3,2-dioxaborolane (125 mg, 1.2 equiv). The crude was purified by flash chromatography (Interchim® 20 μm, CyHex 100% to CyHex/EtOAc 60:40) to obtain Compound 344 (127 mg, 55%) as a white solid.

M/Z (M+H)⁺: 640.5

### Example 144: 6-(4-(3-(3-fluoro-4-(trifluoromethyl)phenyl)-1-isobutyl-1H-pyrrolo[2,3-b]pyridine-6-carbonyl)-3,3-dimethylpiperazin-1-yl)-2,4-dimethylnicotinic acid

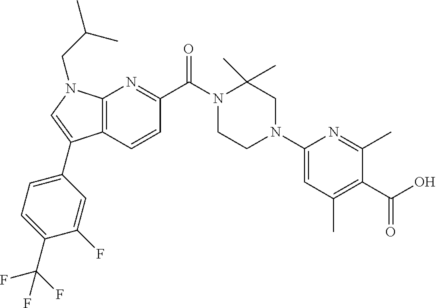 [SMILES: Cc1cc(N2CCN(C(=O)c3ccc4c(-c5ccc(C(F)(F)F)c(F)c5)cn(CC(C)C)c4n3)C(C)(C)C2)nc(C)c1C(=O)O]

Example 144 was prepared according to general procedure (XIb) starting from Compound 344 (53 mg). The crude was purified by flash chromatography (Merck 60®, CyHex 100% to EtOAc 100% to EtOAc/MeOH 80:20), triturated in Et₂O and freeze-dried with EtOH/water to obtain Example 144 (20 mg, 39%) as a white solid.

¹H-NMR (DMSO-d₆, 300 MHz) δ: 0.92 (d, J 6.8 Hz, 6H, (CH₃)₂); 1.52 (s, 6H, (CH₃)₂); 2.18 (s, 3H, CH₃); 2.24-2.30 (m, 4H, CH, CH₃); 3.50-3.53 (m, 2H, CH₂); 3.76-3.78 (m, 2H, CH₂); 3.85 (s, 2H, CH₂); 4.15 (d, J 6.8 Hz, 2H, CH₂); 6.15 (s, 1H, Ar); 7.44 (d, J 8.4 Hz, 1H, Ar); 7.77-7.80 (m, 2H, Ar); 7.85 (d, J 12.9 Hz, 1H, Ar); 8.42 (s, 1H, Ar); 8.53 (d, J 8.4 Hz, 1H, Ar). 1 proton not observed.

M/Z (M+H)⁺: 626.5

### Example 145: 4-(3-(3,4-dimethoxyphenyl)-1-isobutyl-1H-pyrrolo[2,3-b]pyridine-6-carbonyl)-3,3-dimethylpiperazin-2-one

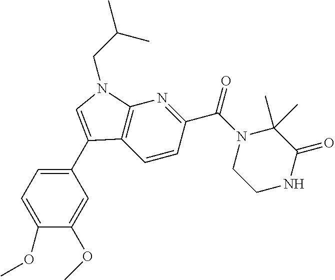 [SMILES: COc1ccc(-c2cn(CC(C)C)c3nc(C(=O)N4CCNC(=O)C4(C)C)ccc23)cc1OC]

Example 145 was prepared according to general procedure (XVIIa) starting from Compound 99 (100 mg) and 2-(3,4-dimethoxyphenyl)-4,4,5,5-tetramethyl-1,3,2-dioxaborolane (78 mg, 1.2 equiv). The crude was purified by flash chromatography (Interchim® 20 μm, CyHex/EtOAc 50:50 to EtOAc 100%) then recrystallized from hot ACN (5 mL) to obtain Example 145 (25 mg, 22%) as a white solid.

¹H-NMR (DMSO-d₆, 300 MHz) δ: 0.87 (d, J 6.9 Hz, 6H, CH(CH₃)₂); 1.72 (s, 6H, C(CH₃)₂); 2.16-2.28 (m, 1H, CH(CH₃)₂); 3.50-3.58 (m, 2H, N—CH₂); 3.79 (s, 3H, O—CH₃); 3.86 (s, 3H, O—CH₃); 4.10 (d, J 7.2 Hz, 2H, CH₂CH); 7.03 (d, J 8.1 Hz, 1H, Ar); 7.20-7.26 (m, 2H, 2 Ar); 7.42 (d, J 8.1 Hz, 1H, Ar); 8.05 (s, 1H, Ar); 8.11 (s, 1H, NH); 8.37 (d, J 8.1 Hz, 1H, Ar). 2 protons not observed.

M/Z (M+H)⁺: 465.3

### Compound 345: tert-butyl 3-(5-(methoxycarbonyl)-4,6-dimethylpyridin-2-yl)-3,8-diazabicyclo[3.2.1]octane-8-carboxylate

Compound 345 was prepared according to general procedure (XXI) starting from methyl 6-chloro-2,4-dimethylnicotinate (137 mg) and tert-butyl 3,8-diazabicyclo[3.2.1]octane-8-carboxylate (160 mg, 1.1 Eq). The crude was purified by flash chromatography (CyHex 100% to CyHex/EtOAc 50:50) to obtain Compound 345 as a white solid (181 mg).

M/Z (M+H)⁺: 376.3

### Compound 346: methyl 6-(3,8-diazabicyclo[3.2.1]octan-3-yl)-2,4-dimethylnicotinate hydrochloride

Compound 346 was prepared according to general procedure (XVIIIb) starting from Compound 345 (180 mg). The reaction mixture was concentrated under reduced to obtain Compound 346 (130 mg, 61% over two steps) as a yellow solid.

M/Z (M+H)⁺: 276.2

### Compound 347: methyl 6-((1R,5S)-8-(1-(tert-butyl)-3-(4-chloro-3-fluorophenyl)-1H-pyrrolo[2,3-b]pyridine-6-carbonyl)-3,8-diazabicyclo[3.2.1]octan-3-yl)-2,4-dimethylnicotinate

Compound 347 was prepared according to general procedure (Xb) starting from Compound 78 (60 mg) and Compound 346 (54 mg, 1.0 equiv), and using N,N-diisopropylethylamine (4.0 equiv). The crude was purified by flash chromatography (CyHex 100% to CyHex/EtOAc 70:30) to obtain Compound 347 (78 mg, 75%) as a yellow solid.

M/Z (M[³⁵Cl]+H)⁺: 604.5

### Example 146: 6-(4-(1-(tert-butyl)-3-(4-chloro-3-fluorophenyl)-1H-pyrrolo[2,3-b]pyridine-6-carbonyl)-2-(methoxymethyl)piperazin-1-yl)-2,4-dimethylnicotinic acid

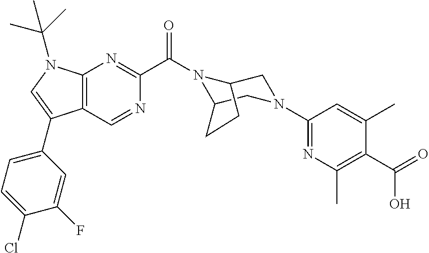 [SMILES: Cc1cc(N2CC3CCC(C2)N3C(=O)c2ncc3c(-c4ccc(Cl)c(F)c4)cn(C(C)(C)C)c3n2)nc(C)c1C(=O)O]

Example 146 was prepared according to general procedure (XIb) starting from Compound 347 (78 mg). The crude was purified by flash chromatography (DCM 100% to DCM/MeOH 85:15) and freeze-dried from MeOH/water to obtain Example 146 (51 mg, 67%) as an off white solid.

¹H-NMR (DMSO-d₆, 300 MHz) δ: 1.67-1.78 (m, 2H, CHaHb-CHaHb); 1.82 (s, 9H, C(CH₃)₃); 1.87-1.99 (m, 2H, CHaHb-CHaHb); 2.25 (s, 3H, CH₃); 2.36 (s, 3H, CH₃); 3.04-3.23 (m, 2H, 2N—CHaHb); 4.15-4.26 (m, 2H, 2N—CHaHb); 4.85-4.92 (m, 1H, N—CH); 4.97-5.06 (m, 1H, N—CH); 6.50 (s, 1H, Ar); 7.57-7.72 (m, 3H, 3 Ar); 7.79-7.85 (m, 1H, Ar); 8.24 (s, 1H, Ar); 8.45 (d, J 8.3 Hz, 1H, Ar). 1 protons not observed.

M/Z (M[³⁵Cl]+H)⁺: 590.5

### Compound 348: methyl 6-((1-(tert-butoxycarbonyl)piperidin-4-yl)(methyl)amino)-2,4-dimethylnicotinate

Compound 348 was prepared according to general procedure (XXI) starting from methyl 6-chloro-2,4-dimethylnicotinate (311 mg) and tert-butyl 4-(methylamino)piperidine-1-carboxylate (400 mg, 1.1 Eq). The crude was purified by flash chromatography (CyHex 100% to CyHex/EtOAc 75:25) to obtain Compound 348 as a white solid (345 mg).

M/Z (M+H)⁺: 378.2

### Compound 349: methyl 2,4-dimethyl-6-(methyl(piperidin-4-yl)amino)nicotinate hydrochloride

Compound 349 was prepared according to general procedure (XVIIIc) starting from Compound 348 (343 mg). The reaction mixture was concentrated under reduced pressure and triturated in Et₂O (3*15 mL) to obtain Compound 349 (260 mg) as a white solid.

M/Z (M+H)⁺: 278.2

### Compound 350: methyl 6-((1-(1-(tert-butyl)-3-(4-chloro-3-fluorophenyl)-1H-pyrrolo[2,3-b]pyridine-6-carbonyl)piperidin-4-yl)(methyl)amino)-2,4-dimethylnicotinate

Compound 350 was prepared according to general procedure (Xa) starting from Compound 78 (60 mg) and Compound 349 (65 mg, 1.2 equiv), and using N,N-diisopropylethylamine (4.0 equiv). The crude was purified by flash chromatography (CyHex 100% to CyHex/EtOAc 65:35) to obtain Compound 350 (88 mg, 88%) as a yellow solid.

M/Z (M[³⁵Cl]+H)⁺: 606.5

### Example 147: 6-((1-(1-(tert-butyl)-3-(4-chloro-3-fluorophenyl)-1H-pyrrolo[2,3-b]pyridine-6-carbonyl)piperidin-4-yl)(methyl)amino)-2,4-dimethyinicotinic acid

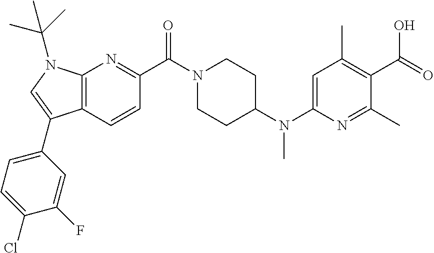 [SMILES: Cc1cc(N(C)C2CCN(C(=O)c3ccc4c(-c5ccc(Cl)c(F)c5)cn(C(C)(C)C)c4n3)CC2)nc(C)c1C(=O)O]

Example 147 was prepared according to general procedure (XIb) starting from Compound 350 (85 mg). The crude was purified by flash chromatography (DCM 100% to DCM/MeOH 80:20) and freeze-dried from MeOH/water to obtain Example 147 (41 mg, 49%) as a white solid.

¹H-NMR (DMSO-d₆, 300 MHz) δ: 1.50-1.63 (s, 1H, one rotamer of CH₂); 1.69-1.77 (m, 2H, CH₂); 1.81 (s, 9H, C(CH₃)₃); 1.87-2.01 (m, 1H, other rotamer of CH₂); 2.25 (s, 3H, CH₃); 2.36 (s, 3H, CH₃); 2.86 (s, 3H, N—CH₃); 2.90-3.01 (m, 1H, one rotamer of CH₂); 3.16-3.27 (m, 1H, other rotamer of CH₂); 4.06-4.18 (m, 1H, one rotamer of CH₂); 4.64-4.76 (m, 1H, other rotamer of CH₂); 4.76-4.87 (m, 1H, N—CH); 6.38 (s, 1H, Ar), 7.46 (d, J 8.3 Hz, 1H, Ar); 7.57-7.70 (m, 2H, Ar); 7.82 (dd, J 11.1 Hz, 1.7 Hz, 1H, Ar); 8.20 (s, 1H, Ar); 8.44 (d, J 8.3 Hz, 1H, Ar). 1 proton not observed.

M/Z (M[³⁵Cl]+H)⁺: 592.4.

### Compound 351: tert-butyl (1S,4S)-5-(5-(methoxycarbonyl)-4,6-dimethylpyridin-2-yl)-2,5-diazabicyclo[2.2.1]heptane-2-carboxylate

Compound 351 was prepared according to general procedure (XXI) starting from methyl 6-chloro-2,4-dimethylnicotinate (211 mg) and tert-butyl methyl(2-(methylamino)ethyl)carbamate (230 mg, 1.1 Eq). The crude was purified by flash chromatography (CyHex 100% to CyHex/EtOAc 50:50) to obtain Compound 351 as a white solid (320 mg, 84%).

M/Z (M+H)⁺: 362.3

### Compound 352: methyl 6-((1S,4S)-2,5-diazabicyclo[2.2.1]heptan-2-yl)-2,4-dimethylnicotinate hydrochloride

A solution of Compound 352 in DCM (15 mL) was treated with HCl in dioxane (4M) (8 mLl). The reaction mixture was stirred at room temperature for 2 hours, then concentrated under reduced pressure. The residue was taken up in DCM (5 mL) precipitated in Et2O (40 mL), and the slurry was concentrated under reduced pressure to obtain Compound 352 (233 mg, 80%) as an off white solid.

M/Z (M+H)⁺: 262.1

### Compound 353: methyl 6-((1S,4S)-5-(1-(tert-butyl)-3-(4-chloro-3-fluorophenyl)-1H-pyrrolo[2,3-b]pyridine-6-carbonyl)-2,5-diazabicyclo[2.2.1]heptan-2-yl)-2,4-dimethylnicotinate

Compound 353 was prepared according to general procedure (Xa) starting from Compound 78 (60 mg) and Compound 352 (57 mg, 1.1 equiv), and using N,N-diisopropylethylamine (4.0 equiv). The crude was purified by flash chromatography (CyHex 100% to CyHex/EtOAc 70:30) to obtain Compound 353 (91 mg, 89%) as a yellow solid.

M/Z (M[³⁵Cl]+H)⁺: 590.4

### Example 148: 6-((1S,4S)-5-(1-(tert-butyl)-3-(4-chloro-3-fluorophenyl)-1H-pyrrolo[2,3-b]pyridine-6-carbonyl)-2,5-diazabicyclo[2.2.1]heptan-2-yl)-2,4-dimethyinicotinic acid

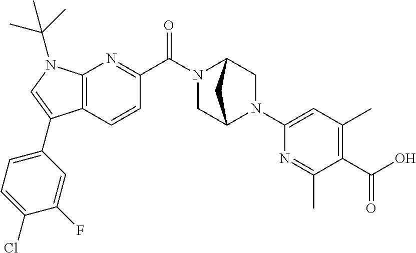 [SMILES: Cc1cc(N2C[C@@H]3C[C@H]2CN3C(=O)c2ccc3c(-c4ccc(Cl)c(F)c4)cn(C(C)(C)C)c3n2)nc(C)c1C(=O)O]

Example 148 was prepared according to general procedure (XIb) starting from Compound 353 (88 mg). The crude was purified by flash chromatography (DCM 100% to DCM/MeOH 80:20) and freeze-dried from MeOH/water to obtain Example 148 (47 mg, 55%) as a white solid.

¹H-NMR (DMSO-d₆, 300 MHz) δ: 1.71 (s, 3.6H, one rotamer of C(CH₃)₃); 1.86 (s, 5.4H, other rotamer of C(CH₃)₃); 2.02 (m, 2H, CH₂); 2.19 (s, 1.2H, one rotamer of CH₃); 2.25 (s, 1.8H, other rotamer of CH₃); 2.31 (s, 1.2H, one rotamer of CH₃); 2.36 (s, 1.8H, other rotamer of CH₃); 3.40-3.53 (m, 0.8H, one rotamer of N—CH₂); 3.57-3.70 (m, 1.2H, other rotamer of N—CH₂); 3.70-3.85 (m, 1.2H, one rotamer of N—CH₂) 3.99 (s, 0.8H, other rotamer of N—CH₂); 4.91 (m, 1H, N—CH); 5.03 (m, 0.4H, one rotamer of N—CH); 5.19 (m, 0.6H, other rotamer N—CH); 6.25 (m, 0.4H, one rotamer of Ar); 6.28 (s, 0.6H, other rotamer of Ar); 7.59-7.76 (m, 3H, 3 Ar); 7.79 (dd, J 11.1 Hz, 1.7 Hz, 0.4H, one rotamer of Ar); 7.83 (dd, J 11.1 Hz, 1.7 Hz, 0.6H, other rotamer of Ar); 8.20 (s, 0.4H, one rotamer of Ar); 8.26 (s, 0.6H, other rotamer of Ar); 8.38 (d, J 8.3 Hz, 0.4H, one rotamer of Ar), 8.46 (d, J 8.3 Hz, 0.6H, other rotamer of Ar). 1 proton not observed.

M/Z (M[³⁵Cl]+H)⁺: 576.4.

### Compound 354: methyl 6-(4-(3-bromo-1-(pyridin-4-ylmethyl)-1H-pyrrolo[2,3-b]pyridine-6-carbonyl)-3,3-dimethylpiperazin-1-yl)-2,4-dimethylnicotinate

Compound 354 was prepared according to general procedure (Xc) starting from Compound 271 (260 μmol), Compound 95 (98 mg) and Et₃N (2.8 equiv). The crude residue was purified by flash chromatography (CyHex 100% to EtOAc 100%) to obtain Compound 354 (130 mg) as a yellow solid. M/Z (M[⁷⁹Br]+H)⁺: 591.4

### Compound 355: methyl 6-(4-(3-(4-chloro-3-fluorophenyl)-1-(pyridin-4-ylmethyl)-1H-pyrrolo[2,3-b]pyridine-6-carbonyl)-3,3-dimethylpiperazin-1-yl)-2,4-dimethylnicotinate

Compound 355 was prepared according to general procedure (XVIIa) starting from Compound 354 (130 mg) and (4-chloro-3-fluorophenyl)boronic acid (46 mg). The crude was purified by flash chromatography (Interchim® 50 μm, CyHex/EtOAc 50:50 to EtOAc 100%), then further purified by preparative HPLC (Column B, 20% to 100% ACN/H₂O), then freeze-dried with water to obtain Compound 355 (40 mg, 27% over 3 steps) as a white solid.

M/Z (M[³⁵Cl]+H)⁺: 641.3

### Example 149: 6-(4-(3-(4-chloro-3-fluorophenyl)-1-(pyridin-4-ylmethyl)-1H-pyrrolo[2,3-b]pyridine-6-carbonyl)-3,3-dimethylpiperazin-1-yl)-2,4-dimethylnicotinic acid

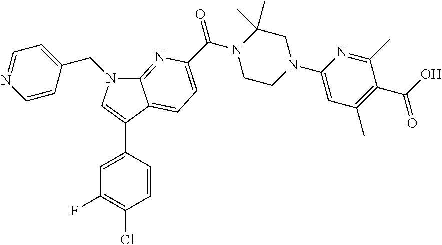 [SMILES: Cc1cc(N2CCN(C(=O)c3ccc4c(-c5ccc(Cl)c(F)c5)cn(Cc5ccncc5)c4n3)C(C)(C)C2)nc(C)c1C(=O)O]

Example 149 was prepared according to general procedure (XIb) starting from Compound 355 (40 mg). The crude was purified by preparative HPLC (Column B, 5% to 100% ACN/H₂O) and freeze-dried from MeOH/water to obtain Example 149 (21 mg, 53%) as a white solid.

¹H-NMR (DMSO-d₆, 300 MHz) δ: 1.48 (s, 6H, 2 CH₃); 2.29 (s, 3H, CH₃); 2.38 (s, 3H, CH₃); 3.19-3.21 (m, 2H, N—CH₂—CH₂); 3.57-3.60 (m, 2H, N—CH₂—CH₂); 3.86 (s, 2H, N—CH₂); 5.63 (s, 2H, Ar—CH₂); 6.18 (s, 1H, Ar); 7.17 (d, J 5.7 Hz, 2H, Ar); 7.44 (d, J 8.1 Hz, 1H, Ar); 7.61-7.72 (m, 2H, Ar); 7.79 (d, J 10.2 Hz, 1H, Ar); 8.38 (s, 1H, Ar); 8.48-8.5 (m, 3H, Ar); 1 proton not observed.

M/Z (M[³⁵Cl]+H)⁺: 627.3

### Compound 356: 3-bromo-1-(pyridin-2-ylmethyl)-1H-pyrrolo[2,3-b]pyridine-6-carbonitrile

To a solution of Compound 23 (300 mg, 1.0 equiv) and 2-(bromomethyl)pyridine hydrobromide (410 mg, 1.2 equiv) in THF (10 mL) was added sodium hydride (189 mg, 60% Wt, 3.5 equiv) at 0° C. The reaction mixture was stirred at 0° C. for 10 min then at 25° C. for 24 hours.

The reaction mixture was quenched with NH₄Cl sat. aq. (40 mL) then extracted with DCM (2*30 mL). The organic layer was washed with brine (20 mL), dried over magnesium sulfate then concentrated to dryness to obtain Compound 356 as a dark orange powder (443 mg).

M/Z (M[⁷⁹Br]+H)⁺: 313.2.

### Compound 357: 3-bromo-1-(pyridin-2-ylmethyl)-1H-pyrrolo[2,3-b]pyridine-6-carboxamide

Compound 357 was prepared according to general procedure (VII) starting from Compound 356 (1.34 mmol) and using potassium carbonate (2.0 equiv) and hydrogen peroxide aqueous 30% (3.0 equiv). The reaction mixture was diluted in water (100 mL) and the precipitate was filtered, and washed with water (20 mL) to obtain Compound 357 (311 mg, 70% over 2 steps) as a yellow solid.

M/Z (M[⁸¹Br]+H)⁺: 333.2.

### Compound 358: methyl 3-bromo-1-(pyridin-2-ylmethyl)-1H-pyrrolo[2,3-b]pyridine-6-carboxylate

Compound 358 was prepared according to general procedure (VIII) starting from Compound 357 (310 mg). The crude was purified by flash chromatography (CyHex 100% to EtOAc 100%) to obtain Compound 358 (140 mg, 43%) as a white solid.

M/Z (M[⁸¹Br]+H)⁺: 348.1

### Compound 359: Lithium 3-bromo-1-(pyridin-2-ylmethyl)-1H-pyrrolo[2,3-b]pyridine-6-carboxylate

To a solution of Compound 358 (140 mg) in a THF (2.0 mL)/Water (2.0 mL) mixture was added Lithium hydroxide monohydrate (43.6 mg, 2.0 equiv). The reaction was stirred at 25° C. for 18 hours. The reaction mixture was concentrated in vacuo, taken up in DCM (10 mL), dried over magnesium sulfate then concentrated to dryness to obtain Compound 359 (183 mg) as a white solid.

M/Z (M[⁸¹Br]+H)⁺: 334.1.

### Compound 360: methyl 6-(4-(3-bromo-1-(pyridin-2-ylmethyl)-1H-pyrrolo[2,3-b]pyridine-6-carbonyl)-3,3-dimethylpiperazin-1-yl)-2,4-dimethylnicotinate

Compound 360 was prepared according to general procedure (Xc) starting from Compound 359 (404 μmol), Compound 95 (98 mg) and Et₃N (5.3 equiv). The crude residue was purified by flash chromatography (CyHex 100% to CyHex/EtOAc 20:80) to obtain Compound 360 (180 mg) as a white solid. M/Z (M[⁸¹Br]+H)⁺: 593.3

### Compound 361: methyl 6-(4-(3-(4-chloro-3-fluorophenyl)-1-(pyridin-2-ylmethyl)-1H-pyrrolo[2,3-b]pyridine-6-carbonyl)-3,3-dimethylpiperazin-1-yl)-2,4-dimethylnicotinate

Compound 361 was prepared according to general procedure (XVIIa) starting from Compound 360 (404 μmol) and (4-chloro-3-fluorophenyl)boronic acid (85 mg). The crude was purified by flash chromatography (Interchim® 50 μm, CyHex 100% to CyHex/EtOAc 50:50) to obtain Compound 361 (110 mg, 43% over 3 steps) as a white solid.

M/Z (M[³⁵Cl]+H)⁺: 641.5

### Example 150: 6-(4-(3-(4-chloro-3-fluorophenyl)-1-(pyridin-2-ylmethyl)-1H-pyrrolo[2,3-b]pyridine-6-carbonyl)-3,3-dimethylpiperazin-1-yl)-2,4-dimethylnicotinic acid

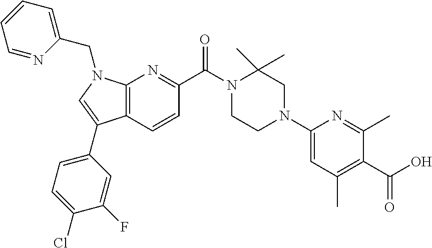 [SMILES: Cc1cc(N2CCN(C(=O)c3ccc4c(-c5ccc(Cl)c(F)c5)cn(Cc5ccccn5)c4n3)C(C)(C)C2)nc(C)c1C(=O)O]

Example 150 was prepared according to general procedure (XIb) starting from Compound 361 (110 mg). The crude was purified by preparative HPLC (Column B, 5% to 100% ACN/H₂O) and freeze-dried from HCl 1M (0.12 mL) and water (100 mL) to obtain Example 150 (44 mg, 39%) as a white solid.

¹H-NMR (DMSO-d₆ 300 MHz) δ: 1.50 (s, 6H, (CH₃)₂); 2.32 (s, 3H, CH₃); 2.44 (s, 3H, CH₃); 3.33-3.37 (m, 2H, CH₂); 3.63-3.67 (m, 2H, CH₂); 3.89 (s, 2H, CH₂); 5.70 (s, 2H, CH₂); 6.43 (bs, 1H, Ar); 7.18 (d, J 8.2 Hz, 1H, Ar); 7.34-7.38 (m, 1H, Ar); 7.43 (d, J 8.2 Hz, 1H, Ar); 7.63-7.66 (m, 2H, Ar); 7.77-7.85 (m, 2H, Ar); 8.37 (s, 1H, Ar); 8.51 (d, J 8.2 Hz, 1H, Ar); 8.54-8.56 (m, 1H, Ar). 1 proton not observed.

M/Z (M[³⁵Cl]+H)⁺: 627.2.

### Compound 362: 6-chloro-3-(4-chloro-3-fluorophenyl)-1-(1-methoxy-2-methylpropan-2-yl)-1H-pyrrolo[2,3-b]pyridine

Compound 362 was prepared according to general procedure (XIII) in THE from Compound 251 (150 mg) and iodomethane (35 μL, 1.3 equiv). The reaction mixture was stirred for 48 hours. The crude was purified by flash chromatography (CyHex 100% to CyHex/EtOAc 75:25) to obtain Compound 362 (130 mg, 83%) as a yellow solid.

M/Z (M[³⁵Cl]+H)⁺: 367.3

### Compound 363: 3-(4-chloro-3-fluorophenyl)-1-(1-methoxy-2-methylpropan-2-yl)-1H-pyrrolo[2,3-b]pyridine-6-carbonitrile

Compound 363 was prepared according to general procedure (Va) starting from Compound 362 (270 mg). The crude was purified by flash chromatography (CyHex 100% to CyHex/EtOAc 70:30) to obtain Compound 363 (220 mg, 84%) as an off white solid.

M/Z (M[³⁵Cl]+H)⁺: 358.2

### Compound 364: methyl 3-(4-chloro-3-fluorophenyl)-1-(1-methoxy-2-methylpropan-2-yl)-1H-pyrrolo[2,3-b]pyridine-6-carboxylate

Compound 364 was obtained (213 mg, 92%), as a yellow solid according to general procedure (VII) starting from Compound 363 (220 mg).

M/Z (M[³⁵Cl]+H)⁺: 376.2

### Compound 365: 3-(4-chloro-3-fluorophenyl)-1-(1-methoxy-2-methylpropan-2-yl)-1H-pyrrolo[2,3-b]pyridine-6-carboxylic acid

Compound 365 prepared according to general procedure (VIII) starting from Compound 364 (144 mg). The crude was purified by flash chromatography (CyHex 100% to CyHex/EtOAc 60:40) to obtain Compound 365 (187 mg, 86%) as a yellow oil.

M/Z (M[³⁵Cl]+H)⁺: 391.2

### Compound 366: 3-(4-chloro-3-fluorophenyl)-1-(1-methoxy-2-methylpropan-2-yl)-1H-pyrrolo[2,3-b]pyridine-6-carboxylic acid

Compound 366 was prepared according to general procedure (IXa) starting from Compound 365 (187 mg) to obtain Compound 366 (148 mg, 82%) as a white solid.

M/Z (M[³⁵Cl]+H)⁺: 377.2

### Example 151: 4-(3-(4-chloro-3-fluorophenyl)-1-(1-methoxy-2-methylpropan-2-yl)-1H-pyrrolo[2,3-b]pyridine-6-carbonyl)-3,3-dimethylpiperazin-2-one

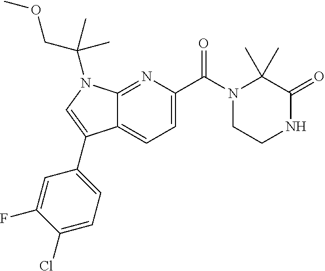 [SMILES: COCC(C)(C)n1cc(-c2ccc(Cl)c(F)c2)c2ccc(C(=O)N3CCNC(=O)C3(C)C)nc21]

Example 151 was prepared according to general procedure (Xa) starting from Compound 366 (48 mg) and 3,3-dimethylpiperazin-2-one (20 mg, 1.2 equiv), and using N,N-diisopropylethylamine (3.0 equiv). The crude was purified by flash chromatography (CyHex 100% to EtOAc 100%) triturated in Et2O, then freeze dried from water/methanol to obtain Example 151 (28 mg, 45%) as an off white solid.

¹H-NMR (DMSO-d₆, 300 MHz) δ: 1.73 (s, 6H, (CH₃)₂); 1.75 (s, 6H, (CH₃)₂); 3.15 (s, 3H, CH₃); 3.54-3.57 (m, 2H, CH₂); 3.95 (s, 2H, CH₂); 7.43 (d, J 8.4 Hz, 1H, Ar); 7.58-7.65 (m, 2H, Ar); 7.78-7.82 (m, 1H, Ar); 8.14 (s, 2H, Ar); 8.42 (d, J 8.4 Hz, 1H, Ar). 2 protons not observed.

M/Z (M[³⁵Cl]+H)⁺: 487.2.

### Compound 367: methyl 6-(4-(3-(4-chloro-3-fluorophenyl)-1-(1-methoxy-2-methylpropan-2-yl)-1H-pyrrolo[2,3-b]pyridine-6-carbonyl)-3,3-dimethylpiperazin-1-yl)-2,4-dimethylnicotinate

Compound 367 was prepared according to general procedure (Xa) starting from Compound 365 (40 mg) and Compound 95 (37 mg, 1.1 equiv), and using N,N-diisopropylethylamine (4.0 equiv). The crude was purified by flash chromatography (CyHex 100% to CyHex/EtOAc 60:40) to obtain Compound 367 (60 mg, 89%) as a white solid.

M/Z (M[³⁵Cl]+H)⁺: 636.6

### Example 152: 6-(4-(3-(4-chloro-3-fluorophenyl)-1-(1-methoxy-2-methylpropan-2-yl)-1H-pyrrolo[2,3-b]pyridine-6-carbonyl)-3,3-dimethylpiperazin-1-yl)-2,4-dimethylnicotinic acid

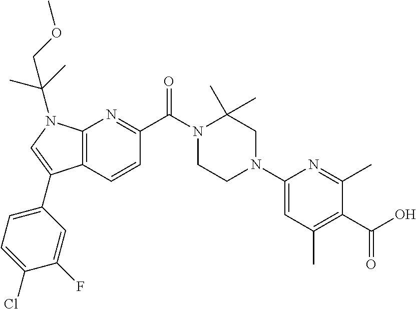 [SMILES: COCC(C)(C)n1cc(-c2ccc(Cl)c(F)c2)c2ccc(C(=O)N3CCN(c4cc(C)c(C(=O)O)c(C)n4)CC3(C)C)nc21]

Example 152 was prepared according to general procedure (XIb) starting from Compound 367 (60 mg). The crude was purified by flash chromatography (DCM 100% to DCM/MeOH 80:20) and freeze dried to obtain Example 152 (35 mg, 60%) as an off-white solid.

¹H-NMR (DMSO-d₆, 300 MHz) δ: 1.54 (s, 6H, (CH₃)₂); 1.77 (s, 6H, (CH₃)₂); 2.25 (s, 3H, CH₃); 2.36 (s, 3H, CH₃); 3.17 (s, 3H, CH₃); 3.50-3.55 (m, 2H, CH₂); 3.79-3.82 (m, 2H, CH₂); 3.91 (s, 2H, CH₂); 3.98 (s, 2H, CH₂); 6.29 (s, 1H, Ar); 7.39 (d, J 8.2 Hz, 1H, Ar); 7.57-7.66 (m, 2H, Ar); 7.78-7.82 (d, J 11.4 Hz, 1H, Ar); 8.14 (s, 1H, Ar); 8.42 (d, J 8.2 Hz, 1H, Ar). 1 proton not observed.

M/Z (M[³⁵Cl]+H)⁺: 622.5.

### Compound 368: methyl (R)-6-((1-(tert-butoxycarbonyl)pyrrolidin-3-yl)amino)-2,4-dimethylnicotinate

Compound 368 was prepared according to general procedure (XXI) starting from methyl 6-chloro-2,4-dimethylnicotinate (500 mg) and tert-butyl (R)-3-aminopyrrolidine-1-carboxylate (513 mg, 1.1 Eq). The crude was purified by flash chromatography (CyHex 100% to CyHex/EtOAc 67:33) to obtain Compound 368 as a yellow solid (650 mg, 74%).

M/Z (M+H)⁺: 350.2

### Compound 369: methyl (R)-2,4-dimethyl-6-(pyrrolidin-3-ylamino)nicotinate hydrochloride

Compound 369 was prepared according to general procedure (XVIIIb) starting from Compound 368 (180 mg). The reaction mixture was concentrated under reduced pressure and triturated in Et₂O (20 mL) to obtain Compound 369 (144 mg, 98%) as a yellow solid.

M/Z (M+H)⁺: 250.2

### Compound 370: methyl (R)-6-((1-(1-(tert-butyl)-3-(4-chloro-3-fluorophenyl)-1H-pyrrolo[2,3-b]pyridine-6-carbonyl)pyrrolidin-3-yl)amino)-2,4-dimethylnicotinate

Compound 370 was prepared according to general procedure (Xa) starting from Compound 78 (70 mg) and Compound 369 (63 mg, 1.1 equiv), and using N,N-diisopropylethylamine (4.0 equiv). The crude was purified by flash chromatography (CyHex 100% to CyHex/EtOAc 50:50) to obtain Compound 370 (86 mg, 74%) as a yellow solid.

M/Z (M[³⁵Cl]+H)⁺: 578.6

### Example 153: (R)-6-((1-(1-(tert-butyl)-3-(4-chloro-3-fluorophenyl)-1H-pyrrolo[2,3-b]pyridine-6-carbonyl)pyrrolidin-3-yl)amino)-2,4-dimethylnicotinic acid

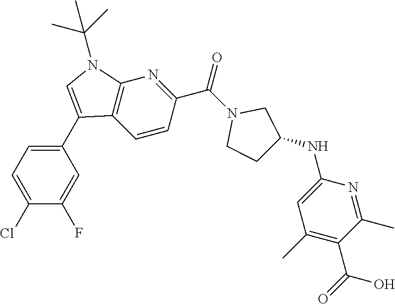 [SMILES: Cc1cc(N[C@@H]2CCN(C(=O)c3ccc4c(-c5ccc(Cl)c(F)c5)cn(C(C)(C)C)c4n3)C2)nc(C)c1C(=O)O]

Example 153 was prepared according to general procedure (XIb) starting from Compound 370 (83 mg). The crude was purified by flash chromatography (DCM 100% to DCM/MeOH 80:20) and freeze-dried from MeOH/water to obtain Example 153 (45 mg, 56%) as a white solid.

¹H-NMR (DMSO-d₆, 300 MHz) δ: 1.75 (s, 5.4H, one rotamer of C(CH₃)₃); 1.82 (s, 3.6H, other rotamer of C(CH₃)₃); 1.91-2.01 (m, 1H, one rotamer of CH₂); 2.12 (s, 1.8H, one rotamer of CH₃); 2.19 (s, 1.2H, other rotamer of CH₃); 2.19-2.22 (m, 1H, other rotamer of CH₂); 2.22 (s, 1.8H, one rotamer of CH₃); 2.34 (s, 1.2H, other rotamer of CH₃); 3.44-3.51 (m, 0.4H, one rotamer of N—CH₂); 3.61-3.71 (m, 0.7H, other rotamer of N—CH₂); 3.77-3.87 (m, 1.1H, one rotamer of N—CH₂); 3.88-3.94 (m, 0.4H, one rotamer of N—CH₂); 3.94-4.08 (m, 1H, other rotamer of N—CH₂); 4.16-4.27 (m, 0.6H, other rotamer of N—CH₂); 4.35-4.47 (m, 1H, N—CH); 6.08 (s, 0.6H, one rotamer of Ar); 6.16 (s, 0.4H, other rotamer of Ar); 6.60-6.70 (m, 0.6H, one rotamer of NH); 6.70-6.79 (m, 0.4H, other rotamer of NH); 7.57-7.67 (m, 1.9H, 2 Ar); 7.69 (d, J 8.3 Hz, 0.6H, one rotamer of Ar); 7.71 (d, J 8.3 Hz, 0.4H, other rotamer of Ar); 7.80 (dd, J 11.1 Hz, 1.7 Hz, 0.6H, one rotamer of Ar); 7.82 (dd, J 11.1 Hz, 1.7 Hz, 0.4H, other rotamer of Ar); 8.20 (s, 0.6H, one rotamer of Ar); 8.23 (s, 0.4H, other rotamer of Ar); 8.40 (d, J 8.3 Hz, 0.6H, one rotamer of Ar); 8.44 (d, J 8.3 Hz, 0.4H, other rotamer of Ar). 1 proton not observed.

M/Z (M[³⁵Cl]+H)⁺: 564.4.

### Compound 371: methyl (S)-6-((1-(tert-butoxycarbonyl)pyrrolidin-3-yl)amino)-2,4-dimethylnicotinate

Compound 371 was prepared according to general procedure (XXI) starting from methyl 6-chloro-2,4-dimethylnicotinate (500 mg) and tert-butyl (S)-3-aminopyrrolidine-1-carboxylate (513 mg, 1.1 Eq). The crude was purified by flash chromatography (CyHex 100% to CyHex/EtOAc 75:25) to obtain Compound 371 as a yellow solid (670 mg, 77%).

M/Z (M+H)⁺: 350.2

### Compound 372: methyl (S)-2,4-dimethyl-6-(pyrrolidin-3-ylamino)nicotinate hydrochloride

Compound 372 was prepared according to general procedure (XVIIIb) starting from Compound 371 (180 mg). The reaction mixture was concentrated under reduced pressure and triturated in Et₂O (20 mL) to obtain Compound 372 (138 mg, 94%) as a yellow solid.

M/Z (M+H)⁺: 250.2

### Compound 373: methyl (S)-6-((1-(1-(tert-butyl)-3-(4-chloro-3-fluorophenyl)-1H-pyrrolo[2,3-b]pyridine-6-carbonyl)pyrrolidin-3-yl)amino)-2,4-dimethylnicotinate

Compound 373 was prepared according to general procedure (Xa) starting from Compound 78 (70 mg) and Compound 372 (63 mg, 1.1 equiv), and using N,N-diisopropylethylamine (4.0 equiv). The crude was purified by flash chromatography (CyHex 100% to CyHex/EtOAc 45:55) to obtain Compound 373 (68 mg, 68%) as a yellow solid.

M/Z (M[³⁵Cl]+H)⁺: 578.4

### Example 154: (S)-6-((1-(1-(tert-butyl)-3-(4-chloro-3-fluorophenyl)-1H-pyrrolo[2,3-b]pyridine-6-carbonyl)pyrrolidin-3-yl)amino)-2,4-dimethylnicotinic acid

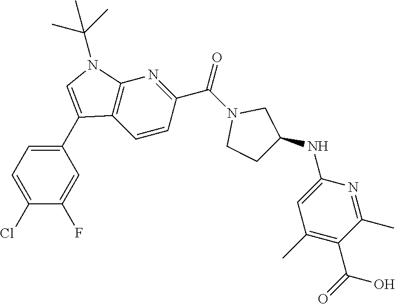 [SMILES: Cc1cc(N[C@H]2CCN(C(=O)c3ccc4c(-c5ccc(Cl)c(F)c5)cn(C(C)(C)C)c4n3)C2)nc(C)c1C(=O)O]

Example 154 was prepared according to general procedure (XIb) starting from Compound 373 (65 mg). The crude was purified by flash chromatography (DCM 100% to DCM/MeOH 80:20) and freeze-dried from MeOH/water to obtain Example 154 (35 mg, 55%) as a white solid.

¹H-NMR (DMSO-d₆, 300 MHz) δ: 1.75 (s, 5.4H, one rotamer of C(CH₃)₃); 1.82 (s, 3.6H, other rotamer of C(CH₃)₃); 1.89-2.02 (m, 1H, one rotamer of CH₂); 2.12 (s, 1.8H, one rotamer of CH₃); 2.18 (s, 1.2H, other rotamer of CH₃); 2.18-2.22 (m, 1H, other rotamer of CH₂); 2.22 (s, 1.8H, one rotamer of CH₃); 2.34 (s, 1.2H, other rotamer of CH₃); 3.44-3.51 (m, 0.4H, one rotamer of N—CH₂); 3.62-3.71 (m, 0.7H, other rotamer of N—CH₂); 3.77-3.86 (m, 1.1H, one rotamer of N—CH₂); 3.86-3.92 (m, 0.4H, one rotamer of N—CH₂); 3.92-4.04 (m, 1H, other rotamer of N—CH₂); 4.14-4.27 (m, 0.7H, other rotamer of N—CH₂); 4.33-4.46 (m, 1H, N—CH); 6.09 (s, 0.6H, one rotamer of Ar); 6.17 (s, 0.4H, other rotamer of Ar); 6.62-6.72 (m, 0.6H, one rotamer of NH); 6.72-6.79 (m, 0.4H, other rotamer of NH); 7.58-7.67 (m, 1.9H, 2 Ar); 7.69 (d, J 8.3 Hz, 0.6H, one rotamer of Ar); 7.71 (d, J 8.3 Hz, 0.4H, other rotamer of Ar); 7.80 (dd, J 11.1 Hz, 1.7 Hz, 0.6H, one rotamer of Ar); 7.82 (dd, J 11.1 Hz, 1.7 Hz, 0.4H, other rotamer of Ar); 8.20 (s, 0.6H, one rotamer of Ar); 8.23 (s, 0.4H, other rotamer of Ar); 8.40 (d, J 8.3 Hz, 0.6H, one rotamer of Ar); 8.44 (d, J 8.3 Hz, 0.4H, other rotamer of Ar). 1 proton not observed.

M/Z (M[³⁵Cl]+H)⁺: 564.5.

### Compound 374: methyl (R)-6-((1-(tert-butoxycarbonyl)pyrrolidin-3-yl)(methyl)amino)-2,4-dimethylnicotinate

Compound 374 was prepared according to general procedure (XIII) in THE starting from Compound 368 (200 mg) and iodomethane (107 μL, 3 eq) to obtain without further purification Compound 374 (210 mg) as a yellow oil.

M/Z (M+H)⁺: 364.2

### Compound 375: methyl (R)-2,4-dimethyl-6-(methyl(pyrrolidin-3-yl)amino)nicotinate hydrochloride

Compound 375 was prepared according to general procedure (XVIIIb) starting from Compound 374 (572 μmol). The reaction mixture was concentrated under reduced pressure and triturated in Et₂O (20 mL) to obtain Compound 375 (200 mg) as a yellow hygroscopic solid.

M/Z (M+H)⁺: 264.2

### Compound 376: methyl (R)-6-((1-(1-(tert-butyl)-3-(4-chloro-3-fluorophenyl)-1H-pyrrolo[2,3-b]pyridine-6-carbonyl)pyrrolidin-3-yl)(methyl)amino)-2,4-dimethylnicotinate

Compound 376 was prepared according to general procedure (Xa) starting from Compound 78 (70 mg) and Compound 375 (60 mg, 1.1 equiv), and using N,N-diisopropylethylamine (4.0 equiv). The crude was purified by flash chromatography (CyHex 100% to CyHex/EtOAc 50:50) to obtain Compound 376 (85 mg, 83%) as a yellow solid.

M/Z (M[³⁵Cl]+H)⁺: 592.5

### Example 155: (R)-6-((1-(1-(tert-butyl)-3-(4-chloro-3-fluorophenyl)-1H-pyrrolo[2,3-b]pyridine-6-carbonyl)pyrrolidin-3-yl)(methyl)amino)-2,4-dimethylnicotinic acid

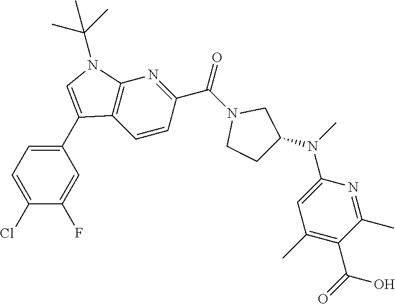 [SMILES: Cc1cc(N(C)[C@@H]2CCN(C(=O)c3ccc4c(-c5ccc(Cl)c(F)c5)cn(C(C)(C)C)c4n3)C2)nc(C)c1C(=O)O]

Example 155 was prepared according to general procedure (XIb) starting from Compound 376 (82 mg). The crude was purified by flash chromatography (DCM 100% to DCM/MeOH 82:18) and freeze-dried from MeOH/water to obtain Example 155 (34 mg, 42%) as a white solid.

¹H-NMR (DMSO-d₆, 300 MHz) δ: 1.74 (s, 5.4H, one rotamer of C(CH₃)₃); 1.83 (s, 3.6H, other rotamer of C(CH₃)₃); 2.05-2.20 (m, 2H, CH₂); 2.21 (s, 1.8H, one rotamer of CH₃); 2.26 (s, 1.2H, other rotamer of CH₃); 2.31 (s, 1.8H, one rotamer of CH₃); 2.38 (s, 1.2H, other rotamer of CH₃); 2.93 (s, 1.8H, one rotamer of N—CH₃); 2.94 (s, 1.2H, other rotamer of N—CH₃); 3.49-3.67 (m, 1H, one rotamer of N—CH₂); 3.75-3.88 (m, 1H, other rotamer of N—CH₂); 3.88-3.97 (m, 1H, one rotamer of N—CH₂); 4.00-4.11 (m, 1H, other rotamer of N—CH₂); 5.21-5.43 (m, 1H, N—CH); 6.38 (s, 0.6H, one rotamer of Ar); 6.44 (s, 0.4H, other rotamer of Ar); 7.57-7.68 (m, 2H, 2 Ar); 7.68-7.74 (m, 1H, Ar); 7.77-7.86 (m, 1H, Ar); 8.20 (s, 0.6H, one rotamer of Ar); 8.24 (s, 0.4H, other rotamer of Ar); 8.43 (d, J 8.3 Hz, 0.6H, one rotamer of Ar); 8.45 (d, J 8.3 Hz, 0.4H, other rotamer of Ar). 1 proton not observed.

M/Z (M[³⁵Cl]+H)⁺: 578.4.

Compound 377: methyl (S)-6-((1-(tert-butoxycarbonyl)pyrrolidin-3-yl)(methyl)amino)-2,4-dimethylnicotinate

Compound 377 was prepared according to general procedure (XIII) in THE starting from Compound 371 (200 mg) and iodomethane (107 μL, 3 eq). The crude was purified by flash chromatography (CyHex 100% to CyHex/EtOAc 75:25) to obtain with Compound 377 (250 mg) as a yellow oil.

M/Z (M+H)⁺: 364.2

### Compound 378: methyl (S)-2,4-dimethyl-6-(methyl(pyrrolidin-3-yl)amino)nicotinate hydrochloride

Compound 378 was prepared according to general procedure (XVIIIb) starting from Compound 377 (572 μmol). The reaction mixture was concentrated under reduced pressure and triturated in Et₂O (20 mL) to obtain Compound 378 (230 mg) as a yellow hygroscopic solid.

M/Z (M+H)⁺: 264.2

### Compound 379: methyl (S)-6-((1-(1-(tert-butyl)-3-(4-chloro-3-fluorophenyl)-1H-pyrrolo[2,3-b]pyridine-6-carbonyl)pyrrolidin-3-yl)(methyl)amino)-2,4-dimethylnicotinate

Compound 379 was prepared according to general procedure (Xa) starting from Compound 78 (60 mg) and Compound 378 (57 mg, 1.1 equiv), and using N,N-diisopropylethylamine (4.0 equiv). The crude was purified by flash chromatography (CyHex 100% to CyHex/EtOAc 50:50) to obtain Compound 379 (85 mg, 83%) as a yellow solid.

M/Z (M[³⁵Cl]+H)⁺: 592.4

### Example 156: (S)-6-((1-(1-(tert-butyl)-3-(4-chloro-3-fluorophenyl)-1H-pyrrolo[2,3-b]pyridine-6-carbonyl)pyrrolidin-3-yl)(methyl)amino)-2,4-dimethylnicotinic acid

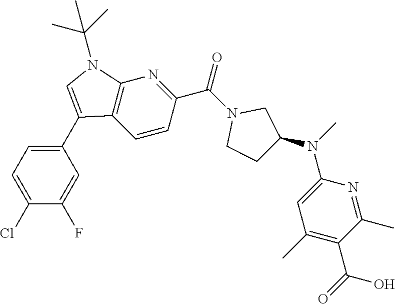 [SMILES: Cc1cc(N(C)[C@H]2CCN(C(=O)c3ccc4c(-c5ccc(Cl)c(F)c5)cn(C(C)(C)C)c4n3)C2)nc(C)c1C(=O)O]

Example 156 was prepared according to general procedure (XIb) starting from Compound 379 (82 mg). The crude was purified by flash chromatography (DCM 100% to DCM/MeOH 82:18) and freeze-dried from MeOH/water to obtain Example 156 (25 mg, 31%) as a white solid.

¹H-NMR (DMSO-d₆, 300 MHz) δ: 1.74 (s, 5.4H, one rotamer of C(CH₃)₃); 1.83 (s, 3.6H, other rotamer of C(CH₃)₃); 2.03-2.19 (m, 2H, CH₂); 2.21 (s, 1.8H, one rotamer of CH₃); 2.26 (s, 1.2H, other rotamer of CH₃); 2.31 (s, 1.8H, one rotamer of CH₃); 2.38 (s, 1.2H, other rotamer of CH₃); 2.93 (s, 1.8H, one rotamer of N—CH₃); 2.94 (s, 1.2H, other rotamer of N—CH₃); 3.48-3.68 (m, 1H, one rotamer of N—CH₂); 3.76-3.88 (m, 1H, other rotamer of N—CH₂); 3.88-3.98 (m, 1H, one rotamer of N—CH₂); 4.00-4.11 (m, 1H, other rotamer of N—CH₂); 5.22-5.42 (m, 1H, N—CH); 6.38 (s, 0.6H, one rotamer of Ar); 6.45 (s, 0.4H, other rotamer of Ar); 7.57-7.68 (m, 2H, 2 Ar); 7.68-7.74 (m, 1H, Ar); 7.77-7.86 (m, 1H, Ar); 8.20 (s, 0.6H, one rotamer of Ar); 8.24 (s, 0.4H, other rotamer of Ar); 8.43 (d, J 8.3 Hz, 0.6H, one rotamer of Ar); 8.45 (d, J 8.3 Hz, 0.4H, other rotamer of Ar). 1 proton not observed.

M/Z (M[³⁵Cl]+H)⁺: 578.5

### Compound 380: methyl 6-(2-(hydroxymethyl)piperazin-1-yl)-2,4-dimethylnicotinate hydrochloride

Compound 380 was prepared according to general procedure (XVIIIc) starting from Compound 308 (200 mg). The reaction mixture was concentrated under reduced pressure and triturated in Et₂O (3*15 mL) to obtain Compound 380 (165 mg) as an off-white solid.

M/Z (M+H)⁺: 280.1

### Compound 381: methyl 6-(4-(1-(tert-butyl)-3-(4-chloro-3-fluorophenyl)-1H-pyrrolo[2,3-b]pyridine-6-carbonyl)-2-(hydroxymethyl)piperazin-1-yl)-2,4-dimethylnicotinate

Compound 381 was prepared according to general procedure (Xa) starting from Compound 78 (175 mg) and Compound 308 (165 mg, 1.1 equiv), and using N,N-diisopropylethylamine (4.0 equiv). The crude was purified by flash chromatography (CyHex 100% to EtOAc 100%) to obtain Compound 381 (292 mg, 95%) as a white solid.

M/Z (M[³⁵Cl]+H)⁺: 608.4

### Example 157: 6-(4-(1-(tert-butyl)-3-(4-chloro-3-fluorophenyl)-1H-pyrrolo[2,3-b]pyridine-6-carbonyl)-2-(hydroxymethyl)piperazin-1-yl)-2,4-dimethylnicotinic acid

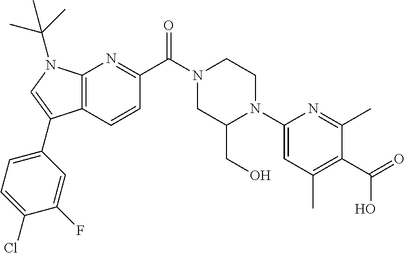 [SMILES: Cc1cc(N2CCN(C(=O)c3ccc4c(-c5ccc(Cl)c(F)c5)cn(C(C)(C)C)c4n3)CC2CO)nc(C)c1C(=O)O]

Example 157 was prepared according to general procedure (XIc) starting from Compound 381 (125 mg). The crude was purified by flash chromatography (DCM 100% to DCM/MeOH 80:20), taken up in EtOAc (100 mL), washed with Sodium Thiosulphate (10% w/w aq, 2*50 mL), the organic layer was washed with NH4Cl, dried over MgSO4, and the filtrate was concentrated under reduced pressure. The residue was freeze-dried from EtOH/water to obtain Example 157 (110 mg, 90%) as a white solid.

¹H-NMR (DMSO-d₆, 300 MHz) δ: 1.81 (s, 9H, C(CH₃)₃); 2.23 (s, 3H, CH₃); 2.34 (s, 3H, CH₃); 3.09-3.23 (m, 1H, CH₂); 3.43-3.73 (m, 3H, CH, CH₂); 4.06-4.30 (m, 3H, CH₂); 4.28-5.00 (m, 3H, CH₂); 6.44 (d, 1H, J 12.6 Hz, Ar); 7.48 (dd, 1H, J 8.1, 2.7 Hz, Ar); 7.60-7.68 (m, 2H, Ar); 7.83 (dd, 1H, J 11.4, 1.5 Hz, Ar); 8.22 (d, 1H, J 3.3 Hz, Ar); 8.46 (d, 1H, J 7.5 Hz, Ar). 1 proton not observed.

M/Z (M[³⁵Cl]+H)⁺: 594.4.

### Compound 382: pyrimidin-2-ylmethyl methanesulfonate

Compound 382 was prepared according to general procedure (XXIII) starting from pyrimidin-2-ylmethanol (200 mg) and methanesulfonyl chloride (250 mg, 1.2 equiv). The crude was purified by flash chromatography (Interchim® 20 μm, CyHex/EtOAc 50:50 to EtOAc 100%) to obtain Compound 382 (257 mg, 75%) as a yellow oil.

M/Z (M+H)⁺: 189.0

### Compound 383: 6-chloro-3-(4-chloro-3-fluorophenyl)-1-(pyrimidin-2-ylmethyl)-1H-pyrrolo[2,3-b]pyridine

Compound 383 was prepared according to general procedure (XIII) in DMF starting from Compound 9 (167 mg) and Compound 382 (200 mg, 1.5 eq). The crude was purified by flash chromatography (CyHex 100% to CyHex/EtOAc 70:30) to obtain Compound 383 (154 mg, 70%) as a white solid.

M/Z (M[³⁵Cl]₂+H)⁺: 373.1

### Compound 384: methyl 6-(4-(3-(4-chloro-3-fluorophenyl)-1-(pyrimidin-2-ylmethyl)-1H-pyrrolo[2,3-b]pyridine-6-carbonyl)-3,3-dimethylpiperazin-1-yl)-2,4-dimethylnicotinate

Compound 384 was prepared according to general procedure (XXX) starting from Compound 383 (104 mg) and Compound 95 (175 mg, 2 eq). The crude was purified by flash chromatography (CyHex 100% to CyHex/EtOAc 40:60) to obtain Compound 384 (135 mg, 51%) as a yellow solid.

M/Z (M[³⁵Cl]+H)⁺: 642.4

### Example 158: 6-(4-(3-(4-chloro-3-fluorophenyl)-1-(pyrimidin-2-ylmethyl)-1H-pyrrolo[2,3-b]pyridine-6-carbonyl)-3,3-dimethylpiperazin-1-yl)-2,4-dimethylnicotinic acid

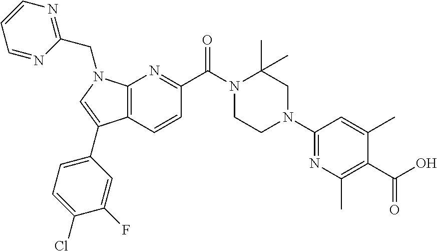 [SMILES: Cc1cc(N2CCN(C(=O)c3ccc4c(-c5ccc(Cl)c(F)c5)cn(Cc5ncccn5)c4n3)C(C)(C)C2)nc(C)c1C(=O)O]

Example 158 was prepared according to general procedure (XIc) starting from Compound 384 (102 mg). The crude was purified by flash chromatography (DCM 100% to DCM/MeOH 90:10) then freeze-dried from EtOH/water to obtain Example 158 (56 mg, 45%) as an orange solid.

¹H-NMR (DMSO-d₆, 300 MHz) δ: 1.46 (s, 6H, 2 CH₃); 2.30 (s, 3H, CH₃—Ar); 2.38 (s, 3H, CH₃—Ar); 3.16-3.23 (m, 2H, CH₂); 3.50-3.57 (m, 2H, CH₂); 3.83 (s, 2H, CH₂); 5.80 (s, 2H, CH₂—Ar); 6.20 (s, 1H, Ar); 7.42-7.46 (m, 2H, Ar); 7.65-7.66 (m, 2H, Ar); 7.77-7.80 (m, 1H, Ar); 8.36 (s, 1H, Ar); 8.50 (d, J 8.3 Hz, 1H, Ar); 8.75-8.77 (m, 2H, Ar) 1 proton not observed.

M/Z (M[³⁵Cl]+H)⁺: 628.4.

### Compound 385: 4-(chloromethyl)-2,5-dimethyloxazole

Compound 385 was prepared according to general procedure (XXIII) starting from (2,5-dimethyloxazol-4-yl)methanol (200 mg) and methanesulfonyl chloride (216 mg, 1.2 equiv). The crude was purified by flash chromatography (CyHex 100% to CyHex/EtOAc 50:50) to obtain Compound 385 (83 mg, 36%) as a clear oil.

M/Z (M+H)⁺: 145.8

### Compound 386: 4-((6-chloro-3-(4-chloro-3-fluorophenyl)-1H-pyrrolo[2,3-b]pyridin-1-yl)methyl)-2,5-dimethyloxazole

Compound 386 was prepared according to general procedure (XIII) in DMF starting from Compound 9 (167 mg) and 385 (83 mg, 1.5 eq). The crude was purified by flash chromatography (CyHex 100% to CyHex/EtOAc 80:20) to obtain Compound 386 (112 mg, 76%) as a white solid.

M/Z (M[³⁵Cl]₂+H)⁺: 390.1

### Compound 387: methyl 6-(4-(3-(4-chloro-3-fluorophenyl)-1-((2,5-dimethyloxazol-4-yl)methyl)-1H-pyrrolo[2,3-b]pyridine-6-carbonyl)-3,3-dimethylpiperazin-1-yl)-2,4-dimethylnicotinate

Compound 387 was prepared according to general procedure (XXX) starting from Compound 386 (112 mg) and Compound 95 (175 mg, 2 eq). The crude was purified by flash chromatography (CyHex 100% to CyHex/EtOAc 60:40) to obtain Compound 387 (128 mg, 68%) as a yellow oil.

M/Z (M[³⁵Cl]+H)⁺: 659.4

### Example 159: 6-(4-(3-(4-chloro-3-fluorophenyl)-1-((2,5-dimethyloxazol-4-yl)methyl)-1H-pyrrolo[2,3-b]pyridine-6-carbonyl)-3,3-dimethylpiperazin-1-yl)-2,4-dimethylnicotinic acid

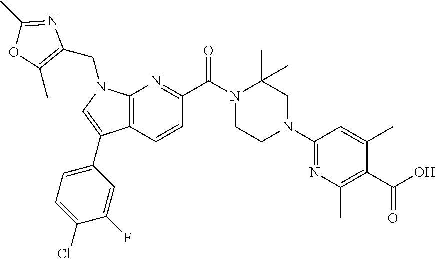 [SMILES: Cc1nc(Cn2cc(-c3ccc(Cl)c(F)c3)c3ccc(C(=O)N4CCN(c5cc(C)c(C(=O)O)c(C)n5)CC4(C)C)nc32)c(C)o1]

Example 159 was prepared according to general procedure (XIb) starting from Compound 387 (102 mg). The crude was triturated in ACN (2*2 mL) then in Et2O (2*2 mL), the solids were dissolved in DCM (10 mL) and the solution was concentrated under reduced pressure to obtain Example 159 (85 mg, 68%) as a white solid.

¹H-NMR (DMSO-d₆, 300 MHz) δ: 1.54 (s, 6H, 2 CH₃); 2.26 (s, 6H, 2 CH₃—Ar); 2.36 (s, 3H, CH₃—Ar); 2.38 (s, 3H, CH₃—Ar); 3.50-3.53 (m, 2H, CH₂); 3.74-3.78 (m, 2H, CH₂); 3.92 (s, 2H, CH₂); 5.31 (s, 2H, N—CH₂—Ar); 6.34 (s, 1H, Ar); 7.39 (d, J 8.2 Hz, 1H, Ar); 7.60-7.67 (m, 2H, Ar); 7.76-7.80 (m, 1H, Ar); 8.23 (s, 1H, Ar); 8.46 (d, J 8.2 Hz, 1H, Ar); 12.68 (s, 1H, COOH).

M/Z (M[³⁵Cl]+H)⁺: 645.4.

### Compound 388: methyl 6-(4-(3-(4-chloro-3-fluorophenyl)-1-(cyclopropylmethyl)-1H-pyrrolo[2,3-b]pyridine-6-carbonyl)-3,3-dimethylpiperazin-1-yl)-2,4-dimethylnicotinate

Compound 388 was prepared according to general procedure (XXX) starting from Compound 12 (153 mg) and Compound 95 (287 mg, 2 eq). The crude was purified by flash chromatography (CyHex 100% to CyHex/EtOAc 70:30) to obtain Compound 388 (185 mg, 67%) as a yellow solid.

M/Z (M[³⁵Cl]+H)⁺: 604.5

### Example 160: 6-(4-(3-(4-chloro-3-fluorophenyl)-1-(cyclopropylmethyl)-1H-pyrrolo[2,3-b]pyridine-6-carbonyl)-3,3-dimethylpiperazin-1-yl)-2,4-dimethylnicotinic acid

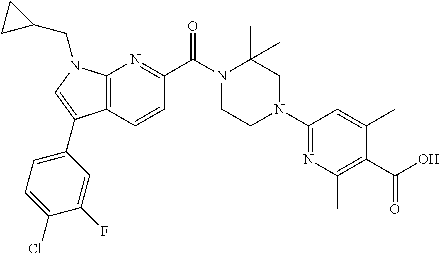 [SMILES: Cc1cc(N2CCN(C(=O)c3ccc4c(-c5ccc(Cl)c(F)c5)cn(CC5CC5)c4n3)C(C)(C)C2)nc(C)c1C(=O)O]

Example 160 was prepared according to general procedure (XIb) starting from Compound 388 (102 mg). The crude was triturated in ACN (2*2 mL) then in Et2O (2*2 mL), the solids were dissolved in DCM (10 mL) and the solution was concentrated under reduced pressure, then freeze dried in EtOH/H2O (1:5, 150 mL) to obtain Example 160 (90 mg, 50%) as a white solid.

¹H-NMR (DMSO-d₆, 300 MHz) δ: 0.46-0.59 (m, 4H, 2 CH₂ CyPr); 1.29-1.41 (m, 1H, CH CyPr); 1.54 (s, 6H, 2 CH₃); 2.27 (s, 3H, CH₃—Ar); 2.39 (s, 3H, CH₃—Ar); 3.53-3.57 (m, 2H, CH₂); 3.77-3.81 (m, 2H, CH₂); 3.92 (s, 2H, CH₂); 4.19 (d, J 7.1 Hz, 2H, CH₂—CyPr); 6.33 (s, 1H, Ar); 7.41 (d, J 8.2 Hz, 1H, Ar); 7.61-7.68 (m, 2H, Ar); 7.76-7.80 (m, 1H, Ar); 8.33 (s, 1H, Ar); 8.46 (d, J 8.2 Hz, 1H, Ar). 1 proton not observed.

M/Z (M[³⁵Cl]+H)⁺: 590.5

### Compound 389: 6-chloro-3-(4-chloro-3-fluorophenyl)-1-((6-methoxypyridin-2-yl)methyl)-1H-pyrrolo[2,3-b]pyridine

Compound 389 was prepared according to general procedure (IV) starting from Compound 3 (500 mg) and (6-methoxypyridin-2-yl)methanamine (542 mg, 2.5 equiv). The crude was purified by flash chromatography (CyHex 100% to CyHex/EtOAc 90:10) to obtain Compound 389 (240 mg, 38%) as a yellow oil.

M/Z (M[³⁵Cl]₂+H)⁺: 402.7

### Compound 390: 3-(4-chloro-3-fluorophenyl)-1-((6-methoxypyridin-2-yl)methyl)-1H-pyrrolo[2,3-b]pyridine-6-carbonitrile

Compound 390 was prepared according to general procedure (V) starting from Compound 389 (240 mg). The crude was purified by flash chromatography (CyHex 100% to CyHex/EtOAc 80:20) to obtain Compound 390 (104 mg, 44%) as a yellow solid.

M/Z (M[³⁵Cl]+H)⁺: 393.8

### Compound 391: 3-(4-chloro-3-fluorophenyl)-1-((6-methoxypyridin-2-yl)methyl)-1H-pyrrolo[2,3-b]pyridine-6-carboxamide

Compound 391 was obtained (125 mg), as a yellow solid according to general procedure (VII) starting from Compound 390 (104 mg).

M/Z (M[³⁵Cl]+H)⁺: 411.2

### Compound 392: methyl 3-(4-chloro-3-fluorophenyl)-1-((6-methoxypyridin-2-yl)methyl)-1H-pyrrolo[2,3-b]pyridine-6-carboxylate

Compound 392 prepared according to general procedure (VIII) starting from Compound 391 (265 μmol). The crude was purified by flash chromatography (CyHex 100% to CyHex/EtOAc 70:30) to obtain Compound 392 (77 mg, 71% over 2 steps) as a yellow solid.

M/Z (M[³⁵Cl]+H)⁺: 426.2

### Compound 393: 3-(4-chloro-3-fluorophenyl)-1-((6-methoxypyridin-2-yl)methyl)-1H-pyrrolo[2,3-b]pyridine-6-carboxylic acid

Compound 393 was prepared according to general procedure (IXa) starting from Compound 392 (75 mg) to obtain Compound 393 (65 mg, 90%) as a white solid.

¹H-NMR (DMSO-d₆, 300 MHz) δ: 3.76 (s, 3H, OCH₃); 5.64 (s, 2H, CH₂—Ar); 6.33 (s, 1H, Ar); 6.60 (d, J 7.5 Hz, 1H, Ar); 6.71 (d, J 7.5 Hz, 1H, Ar); 7.57-7.72 (m, 3H, Ar); 7.80 (d, J 10.5 Hz, 1H, Ar); 7.94 (d, J 8.2 Hz, 1H, Ar); 8.45 (s, 1H, Ar); 8.53 (d, J 8.2 Hz, 1H, Ar). 1 proton not observed.

### Compound 394: methyl 6-(4-(3-(4-chloro-3-fluorophenyl)-1-((6-methoxypyridin-2-yl)methyl)-1H-pyrrolo[2,3-b]pyridine-6-carbonyl)-3,3-dimethylpiperazin-1-yl)-2,4-dimethylnicotinate

Compound 394 was prepared according to general procedure (Xa) starting from Compound 393 (62 mg) and Compound 95 (52 mg, 1.1 equiv), and using N,N-diisopropylethylamine (4.0 equiv). The crude was purified by flash chromatography (Merck 60®, CyHex 100% to CyHex/EtOAc 62:38) to obtain Compound 394 (92 mg, 91%) as a white solid.

M/Z (M[³⁵Cl]+H)⁺: 671.4

### Example 161: 6-(4-(3-(4-chloro-3-fluorophenyl)-1-((6-methoxypyridin-2-yl)methyl)-1H-pyrrolo[2,3-b]pyridine-6-carbonyl)-3,3-dimethylpiperazin-1-yl)-2,4-dimethylnicotinic acid

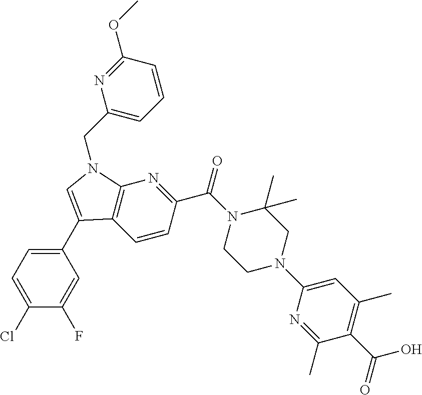 [SMILES: COc1cccc(Cn2cc(-c3ccc(Cl)c(F)c3)c3ccc(C(=O)N4CCN(c5cc(C)c(C(=O)O)c(C)n5)CC4(C)C)nc32)n1]

Example 161 was prepared according to general procedure (XIb) starting from Compound 394 (90 mg). The crude was purified by flash chromatography (DCM 100% to DCM/MeOH 80:20) and freeze dried from EtOH/water to obtain Example 161 (25 mg, 28%) as a white solid.

¹H-NMR (DMSO-d₆, 300 MHz) δ: 1.49 (s, 6H, 2 CH₃); 2.26 (s, 3H, CH₃); 2.36 (s, 3H, CH₃); 3.26 (t, J 5.2 Hz, 2H, CH₂); 3.63 (t, J 5.2 Hz, 2H, CH₂); 3.72 (s, 3H, O—CH₃); 3.85 (s, 2H, CH₂); 5.59 (s, 2H, N—CH₂); 6.16 (s, 1H, Ar); 6.59 (d, J 7.3 Hz, 1H, Ar); 6.71 (d, J 8.3 Hz, 1H, Ar); 7.43 (d, J 8.3 Hz, 1H, Ar); 7.61-7.69 (m, 3H, 3 Ar), 7.79 (d, J 11.1 Hz, 1H, Ar); 8.37 (s, 1H, Ar); 8.52 (d, J 8.3 Hz, 1H, Ar). 1 proton not observed.

M/Z (M[³⁵Cl]+H)⁺:657.5

### Compound 395: methyl 6-(1-(tert-butoxycarbonyl)-N-methylpiperidine-4-carboxamido)-2,4-dimethylnicotinate

Compound 395 was prepared according to general procedure (XIII) in DMA from Compound 281 (200 mg) and iodomethane (94 mg, 1.3 equiv). The reaction mixture was stirred for 1 hours. The crude was purified by flash chromatography (Merck 60®, CyHex 100% to CyHex/EtOAc 60:40) to obtain Compound 395 (230 mg) as a yellow solid.

M/Z (M+H)⁺: 406.2

### Compound 396: methyl 6-(2-(hydroxymethyl)piperazin-1-yl)-2,4-dimethylnicotinate hydrochloride

Compound 396 was prepared according to general procedure (XVIIIb) starting from Compound 395 (207 mg). The reaction mixture was concentrated under reduced pressure to obtain Compound 396 (190 mg) as a white solid.

M/Z (M+H)⁺: 306.3

### Compound 397: methyl 6-(1-(1-(tert-butyl)-3-(4-chloro-3-fluorophenyl)-1H-pyrrolo[2,3-b]pyridine-6-carbonyl)-N-methylpiperidine-4-carboxamido)-2,4-dimethylnicotinate

Compound 397 was prepared according to general procedure (Xa) starting from Compound 78 (60 mg) and Compound 396 (65 mg, 1.1 equiv), and using N,N-diisopropylethylamine (4.0 equiv). The crude was purified by flash chromatography (Merck 60®, CyHex 100% to CyHex/EtOAc 60:40) to obtain Compound 397 (77 mg, 70%) as a yellow solid.

M/Z (M[³⁵Cl]+H)⁺: 634.5.

### Example 162: 1-(1-(tert-butyl)-3-(4-chloro-3-fluorophenyl)-1H-pyrrolo[2,3-b]pyridine-6-carbonyl)piperidine-4-carboxylic acid

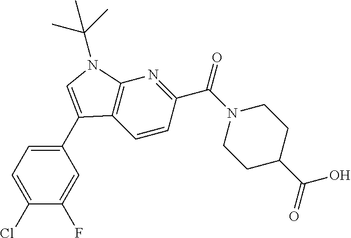 [SMILES: CC(C)(C)n1cc(-c2ccc(Cl)c(F)c2)c2ccc(C(=O)N3CCC(C(=O)O)CC3)nc21]

Example 162 was prepared according to general procedure (XIb) starting from Compound 397 (74 mg). The crude taken up in water, acidified to pH=3 with HCl (1M, aqueous), and extracted with EtOAc (3*30 mL). The organics were washed with brine, dried over MgSO₄, concentrated under reduced pressure then freeze dried from EtOH/water (1:9) to obtain Example 162 (47 mg, 88%) as a white solid.

¹H-NMR (DMSO-d₆, 300 MHz) δ: 1.50-1.68 (m, 2H, CH₂); 1.79 (s, 9H, C(CH₃)₃); 1.83-1.87 (m, 1H, CHaHb); 1.92-2.02 (m, 1H, CHₐH_{b}); 2.54-2.65 (m, 1H, CH—COOH); 2.97-3.08 (m, 1H, N—CHₐH_{b}); 3.15-3.26 (m, 1H, N—CHₐH_{b}); 3.90-4.00 (m, 1H, N—CHₐH_{b}); 4.34-4.44 (m, 1H, N—CHₐH_{b}); 7.41 (d, J 8.3 Hz, 1H, Ar); 7.59-7.67 (m, 2H, 2 Ar); 7.82 (dd, J 11.1 Hz, 1.7 Hz, 1H, Ar); 8.20 (s, 1H, Ar); 8.42 (d, J 8.3 Hz, 1H, Ar); 12.34 (s, 1H, COOH).

M/Z (M[³⁵Cl]+H)⁺: 458.4.

### Example 163: 2-(1-(1-(tert-butyl)-3-(4-chloro-3-fluorophenyl)-1H-pyrrolo[2,3-b]pyridine-6-carbonyl)piperidin-4-yl)acetamide

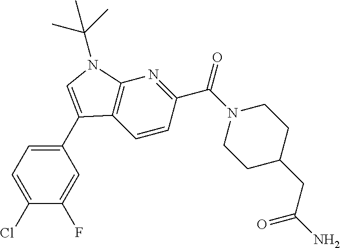 [SMILES: CC(C)(C)n1cc(-c2ccc(Cl)c(F)c2)c2ccc(C(=O)N3CCC(CC(N)=O)CC3)nc21]

Example 163 was prepared according to general procedure (Xa) starting from Example 125 (60 mg) and ammonia (0.5 M in dioxane, 5 eq). The crude was purified by preparative HPLC (Column B, 20% to 100% ACN/H₂O) and freeze-dried from EtOH/water (1:9) to obtain Example 163 (40 mg, 67%) as a white solid.

¹H-NMR (DMSO-d₆, 300 MHz) δ: 1.10-1.32 (m, 2H, CH₂); 1.58-1.69 (m, 1H, CHₐ—H_{b}); 1.71-1.78 (m, 1H, CHₐH_{b}); 1.80 (s, 9H, C(CH₃)₃); 1.92-2.10 (m, 3H, CH+CH₂CO); 2.84 (t, J 12.2 Hz, 1H, N—CHₐH_{b}); 3.1 (t, J 12.2 Hz, 1H, N—CHₐH_{b}); 3.98 (d, J 12.2 Hz, 1H, N—CHₐH_{b}); 4.50 (d, J 12.2 Hz, 1H, N—CHₐH_{b}); 6.79 (s, 1H, NHₐH_{b}); 7.28 (s, 1H, NHₐH_{b}); 7.39 (d, J 8.3 Hz, 1H, Ar); 7.59-7.67 (m, 2H, 2 Ar); 7.82 (dd, J 11.1 Hz, 1.7 Hz, 1H, Ar); 8.20 (s, 1H, Ar); 8.42 (d, J 8.3 Hz, 1H, Ar).

M/Z (M[³⁵Cl]+H)⁺: 471.3.

### Example 164: 2-(1-(1-(tert-butyl)-3-(4-chloro-3-fluorophenyl)-1H-pyrrolo[2,3-b]pyridine-6-carbonyl)piperidin-4-yl)-N,N-dimethylacetamide

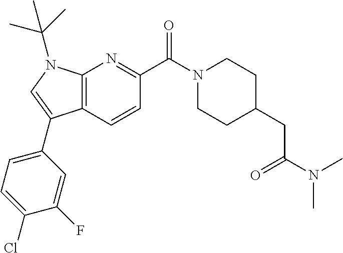 [SMILES: CN(C)C(=O)CC1CCN(C(=O)c2ccc3c(-c4ccc(Cl)c(F)c4)cn(C(C)(C)C)c3n2)CC1]

Example 164 was prepared according to general procedure (Xa) starting from Example 125 (60 mg) and dimethylamine hydrochloride (5 eq) and using N,N-diisopropylethylamine (8.0 equiv). The crude was purified by flash chromatography (CyHex 100% to EtOAc 100%) and freeze-dried from EtOH/water (1:9) to obtain Example 164 (19 mg, 28%) as a white solid.

¹H-NMR (DMSO-d₆, 300 MHz) δ: 1.14-1.31 (m, 2H, CH₂); 1.61-1.73 (m, 1H, CHₐ—H_{b}); 1.76-1.88 (m, 10H, CHₐH_{b}+C(CH₃)₃); 1.97-2.10 (m, 1H, CH); 2.25-2.33 (m, 2H, CH₂); 2.78-2.91 (m, 4H, N—CHₐH_{b}+CO—N—CH₃); 2.96 (s, 3H, CO—N—CH₃); 3.10 (t, J 12.2 Hz, 1H, N—CHₐH_{b}); 4.00 (d, J 12.2 Hz, 1H, N—CHₐH_{b}); 4.51 (d, J 12.2 Hz, 1H, N—CHₐH_{b}); 7.39 (d, J 8.3 Hz, 1H, Ar); 7.59-7.67 (m, 2H, 2 Ar); 7.81 (dd, J 11.1 Hz, 1.7 Hz, 1H, Ar); 8.19 (s, 1H, Ar); 8.42 (d, J 8.3 Hz, 1H, Ar).

M/Z (M[³⁵Cl]+H)⁺: 499.5.

### Compound 398: (R)-1-(sec-butyl)-6-chloro-3-(4-chloro-3-fluorophenyl)-1H-pyrrolo[2,3-b]pyridine

Compound 398 was prepared according to general procedure (IV) starting from Compound 3 (500 mg) and (R)-butan-2-amine (287 mg, 2.5 equiv). The crude was purified by flash chromatography (CyHex 100% to CyHex/EtOAc 90:10) to obtain Compound 398 (281 mg, 53%) as a yellow solid.

M/Z (M[³⁵Cl₂]+H)⁺: 337.6

### Compound 399: (R)-1-(sec-butyl)-3-(4-chloro-3-fluorophenyl)-1H-pyrrolo[2,3-b]pyridine-6-carbonitrile

Compound 399 was prepared according to general procedure (V) starting from Compound 398 (240 mg). The crude was purified by flash chromatography (CyHex 100% to CyHex/EtOAc 90:10) to obtain Compound 399 (169 mg, 62%) as a beige solid.

M/Z (M[³⁵Cl]+H)⁺: 328.7

### Compound 400: (R)-1-(sec-butyl)-3-(4-chloro-3-fluorophenyl)-1H-pyrrolo[2,3-b]pyridine-6-carboxamide

Compound 400 was obtained (217 mg), as a yellow solid according to general procedure (VII) starting from Compound 399 (169 mg).

M/Z (M[³⁵Cl]+H)⁺: 346.1

### Compound 401: methyl (R)-1-(sec-butyl)-3-(4-chloro-3-fluorophenyl)-1H-pyrrolo[2,3-b]pyridine-6-carboxylate

Compound 401 prepared according to general procedure (VIII) starting from Compound 400 (515 μmol). The crude was purified by flash chromatography (Interchim®20 μm, CyHex 100% to CyHex/EtOAc 80:20) to obtain Compound 401 (136 mg, 73% over 2 steps) as a white solid.

M/Z (M[³⁵Cl]+H)⁺: 361.2

### Compound 402: (R)-1-(sec-butyl)-3-(4-chloro-3-fluorophenyl)-1H-pyrrolo[2,3-b]pyridine-6-carboxylic acid

Compound 402 was prepared according to general procedure (IXa) starting from Compound 401 (136 mg) to obtain Compound 402 (119 mg, 91%) as a white solid.

M/Z (M[³⁵Cl]+H)⁺: 347.1

### Compound 403: methyl (R)-6-(4-(1-(sec-butyl)-3-(4-chloro-3-fluorophenyl)-1H-pyrrolo[2,3-b]pyridine-6-carbonyl)-3,3-dimethylpiperazin-1-yl)-2,4-dimethylnicotinate

Compound 403 was prepared according to general procedure (Xa) starting from Compound 402 (60 mg) and Compound 95 (60 mg, 1.1 equiv), and using N,N-diisopropylethylamine (4.0 equiv). The crude was purified by flash chromatography (Merck 60®, CyHex 100% to CyHex/EtOAc 75:25) to obtain Compound 403 (89 mg) as a white solid.

M/Z (M[³⁵Cl]+H)⁺: 606.4

### Example 165: (R)-6-(4-(1-(sec-butyl)-3-(4-chloro-3-fluorophenyl)-1H-pyrrolo[2,3-b]pyridine-6-carbonyl)-3,3-dimethylpiperazin-1-yl)-2,4-dimethylnicotinic acid

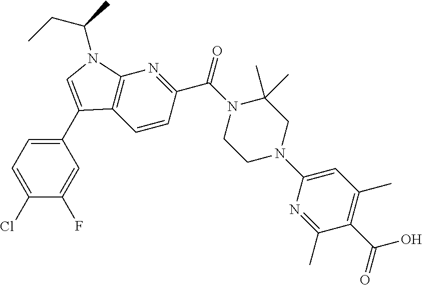 [SMILES: CC[C@@H](C)n1cc(-c2ccc(Cl)c(F)c2)c2ccc(C(=O)N3CCN(c4cc(C)c(C(=O)O)c(C)n4)CC3(C)C)nc21]

Example 165 was prepared according to general procedure (XIb) starting from Compound 403 (90 mg). The crude taken up in water, acidified to pH=3 with HCl (1M, aqueous), and extracted with EtOAc (3*30 mL). The organics were washed with brine, dried over MgSO₄, concentrated, triturated in ACN then in MeOH, and under reduced pressure then freeze dried from EtOH/water (1:9) to obtain Example 165 (47 mg, 88%) as a white solid.

¹H-NMR (DMSO-d₆, 300 MHz) δ: 0.77 (t, J 7.3 Hz, N—CH(CH₃)—CH₂—CH₃); 1.53-1.55 (m, 9H, 2*CH₃+N—CH(CH₃)—CH₂—CH₃); 1.85-2.02 (m, 2H, N—CH(CH₃)—CH₂—CH₃); 2.26 (s, 3H, CH₃); 2.38 (s, 3H, CH₃); 3.49-3.61 (m, 2H, N—CH₂); 3.75-3.83 (m, 2H, N—CH₂); 3.91 (s, 2H, N—CH₂); 4.87-4.98 (m, 1H, N—CH(CH₃)—CH₂—CH₃); 6.33 (s, 1H, Ar); 7.40 (d, J 8.3 Hz, 1H, Ar); 7.60-7.69 (m, 2H, 2 Ar); 7.82 (dd, J 11.1 Hz, 1.7 Hz, 1H, Ar); 8.39 (s, 1H, Ar); 8.46 (d, J 8.3 Hz, 1H, Ar); 12.72 (bs, 1H, COOH).

M/Z (M[³⁵Cl]+H)⁺: 592.3

### Compound 404: 6-chloro-3-(4-chloro-3-fluorophenyl)-1-(1,3-dimethoxypropan-2-yl)-1H-pyrrolo[2,3-b]pyridine

Compound 404 was prepared according to general procedure (IV) starting from Compound 3 (500 mg) and 1,3-dimethoxypropan-2-amine (468 mg, 2.5 equiv). The crude was purified by flash chromatography (CyHex 100% to CyHex/EtOAc 90:10) to obtain Compound 404 (252 mg, 42%) as a white solid.

M/Z (M[³⁵Cl₂]+H)⁺: 383.7

### Compound 405: 3-(4-chloro-3-fluorophenyl)-1-(1,3-dimethoxypropan-2-yl)-1H-pyrrolo[2,3-b]pyridine-6-carbonitrile

Compound 405 was prepared according to general procedure (V) starting from Compound 404 (252 mg). The crude was purified by flash chromatography (CyHex 100% to CyHex/EtOAc 70:30) to obtain Compound 405 (186 mg, 76%) as a beige solid.

M/Z (M[³⁵Cl]+H)⁺: 374.4

### Compound 406: 3-(4-chloro-3-fluorophenyl)-1-(1,3-dimethoxypropan-2-yl)-1H-pyrrolo[2,3-b]pyridine-6-carboxamide

Compound 406 was obtained (252 mg), as a yellow solid according to general procedure (VII) starting from Compound 405 (186 mg).

M/Z (M[³⁵Cl]+H)⁺: 392.6

### Compound 407: methyl 3-(4-chloro-3-fluorophenyl)-1-(1,3-dimethoxypropan-2-yl)-1H-pyrrolo[2,3-b]pyridine-6-carboxylate

Compound 407 prepared according to general procedure (VIII) starting from Compound 406 (498 μmol). The crude was purified by flash chromatography (CyHex 100% to CyHex/EtOAc 50:50) to obtain Compound 407 (153 mg, 76% over 2 steps) as a white solid.

M/Z (M[³⁵Cl]+H)⁺: 407.2

### Compound 408: (3-(4-chloro-3-fluorophenyl)-1-(1,3-dimethoxypropan-2-yl)-1H-pyrrolo[2,3-b]pyridine-6-carboxylic acid

Compound 408 was prepared according to general procedure (IXa) starting from Compound 407 (136 mg) to obtain Compound 408 (153 mg, 94%) as a white solid.

M/Z (M[³⁵Cl]+H)⁺: 393.1

### Compound 409: methyl 6-(4-(3-(4-chloro-3-fluorophenyl)-1-(1,3-dimethoxypropan-2-yl)-1H-pyrrolo[2,3-b]pyridine-6-carbonyl)-3,3-dimethylpiperazin-1-yl)-2,4-dimethylnicotinate

Compound 409 was prepared according to general procedure (Xa) starting from Compound 408 (70 mg) and Compound 95 (62 mg, 1.1 equiv), and using N,N-diisopropylethylamine (4.0 equiv). The crude was purified by flash chromatography (Merck 60®, CyHex 100% to CyHex/EtOAc 70:30) to obtain Compound 409 (78 mg, 67%) as a white solid.

M/Z (M[³⁵Cl]+H)⁺: 652.5

### Example 166: 6-(4-(3-(4-chloro-3-fluorophenyl)-1-(1,3-dimethoxypropan-2-yl)-1H-pyrrolo[2,3-b]pyridine-6-carbonyl)-3,3-dimethylpiperazin-1-yl)-2,4-dimethylnicotinic acid

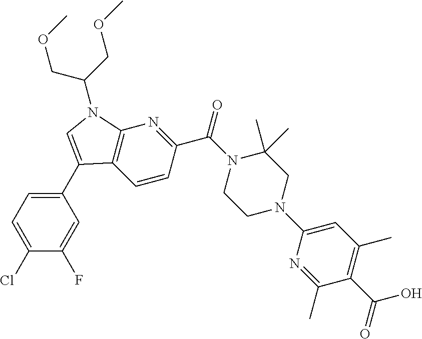 [SMILES: COCC(COC)n1cc(-c2ccc(Cl)c(F)c2)c2ccc(C(=O)N3CCN(c4cc(C)c(C(=O)O)c(C)n4)CC3(C)C)nc21]

Example 166 was prepared according to general procedure (XIb) starting from Compound 409 (78 mg). The crude was purified by flash chromatography (Merck, DCM 100% to DCM/MeOH 90:10) then freeze dried from EtOH/water (1:9) to obtain Example 166 (37 mg, 48%) as a white solid.

¹H-NMR (DMSO-d₆, 300 MHz) δ: 1.54 (s, 6H, 2*CH₃); 2.26 (s, 3H, CH₃); 2.38 (s, 3H, CH₃); 3.25 (s, 6H, 2 O—CH₃); 3.51-3.57 (m, 2H, N—CH₂) 3.76-3.81 (m, 4H, 2 O—CH₂); 3.88-3.94 (m, 4H, 2*N—CH₂); 5.26-5.35 (m, 1H, N—CH—(CH₂—O—CH₃)₂; 6.31 (s, 1H, Ar); 7.41 (d, J 8.3 Hz, 1H, Ar); 7.61-7.69 (m, 2H, 2 Ar); 7.77 (dd, J 11.1 Hz, 1.7 Hz, 1H, Ar); 8.35 (s, 1H, Ar); 8.46 (d, J 8.3 Hz, 1H, Ar). 1 proton not observed.

M/Z (M[³⁵Cl]+H)⁺: 638.5.

### Compound 410: 1′-(tert-butyl) 5-methyl 4,6-dimethyl-3′,6′-dihydro-[2,4′-bipyridine]-1′,5(2′H)-dicarboxylate

To a solution of tert-butyl 4-(4,4,5,5-tetramethyl-1,3,2-dioxaborolan-2-yl)-3,6-dihydropyridine-1(2H)-carboxylate (511 mg, 1.5 equiv) and methyl 6-chloro-2,4-dimethylnicotinate (220 mg) in 1,4-Dioxane (10.0 mL) was added potassium carbonate (1 M in water) (3 equiv). The reaction was sparged with argon for 10 minutes before addition of PdCl2 (dppf) (0.1 equiv). The reaction mixture was heated at 80° C. for 7 hours. The mixture was filtered through a pad of Celite, washed with EtOAc (20 mL). The organic layer was washed with NH4Cl sat. (20 mL), brine (20 mL), dried over magnesium sulfate then concentrated to dryness. The crude was purified by flash chromatography (CyHex 100% to CyHex/EtOAc 60:40) to obtain Compound 410 (368 mg, 96%) as a clear oil.

M/Z (M+H)⁺: 347.3.

### Compound 411: methyl 4,6-dimethyl-1′,2′,3′,6′-tetrahydro-[2,4′-bipyridine]-5-carboxylate hydrochloride

Compound 411 was prepared according to general procedure (XVIIIb) starting from Compound 410 (173 mg). The reaction mixture was concentrated under reduced pressure to obtain Compound 411 (124 mg) as a yellow solid.

M/Z (M+H)⁺: 247.2

### Compound 412: methyl 1′-(1-(tert-butyl)-3-(4-chloro-3-fluorophenyl)-1H-pyrrolo[2,3-b]pyridine-6-carbonyl)-4,6-dimethyl-1′,2′,3′,6′-tetrahydro-[2,4′-bipyridine]-5-carboxylate

Compound 412 was prepared according to general procedure (Xa) starting from Compound 78 (60 mg) and Compound 411 (54 mg, 1.1 equiv), and using N,N-diisopropylethylamine (4.0 equiv). The crude was purified by flash chromatography (Merck 60®, CyHex 100% to CyHex/EtOAc 73:27) to obtain Compound 412 (85 mg) as a white solid.

M/Z (M[³⁵Cl]+H)⁺: 575.3

### Example 167: 1′-(1-(tert-butyl)-3-(4-chloro-3-fluorophenyl)-1H-pyrrolo[2,3-b]pyridine-6-carbonyl)-4,6-dimethyl-1′,2′,3′,6′-tetrahydro-[2,4′-bipyridine]-5-carboxylic acid

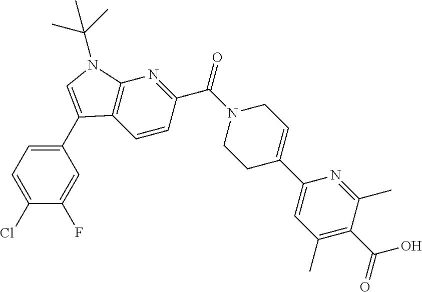 [SMILES: Cc1cc(C2=CCN(C(=O)c3ccc4c(-c5ccc(Cl)c(F)c5)cn(C(C)(C)C)c4n3)CC2)nc(C)c1C(=O)O]

Example 167 was prepared according to general procedure (XIc) starting from Compound 412 (82 mg). The crude was purified by flash chromatography (DCM 100% to DCM/MeOH 80:20) and freeze-dried from EtOH/water to obtain Example 167 (14 mg, 18%) as a yellow solid.

¹H-NMR (DMSO-d₆, 300 MHz) δ: 1.83 (s, 9H, C(CH₃)₃); 2.24 (s, 1.05H, one rotamer of CH₃); 2.26 (s, 1.95H, other rotamer of CH₃); 2.40 (s, 1.05H, one rotamer of CH₃); 2.42 (s, 1.95H, other rotamer of CH₃); 2.66-2.71 (m, 2H, CH₂); 3.79 (t, J 5.2 Hz, 1.3H, other rotamer of N—CH₂); 3.92 (t, J 5.2 Hz, 1.3H, one rotamer of N—CH₂); 4.37 (s, 2H, N—CH₂); 6.54 (s, 0.35H, one rotamer of Ar); 6.72 (s, 0.65H, other rotamer of Ar); 7.15 (s, 0.35H, one rotamer of Ar); 7.19 (s, 0.65H, other rotamer of Ar); 7.48 (d, J 8.3 Hz, 1H, Ar); 7.60-7.69 (m, 2H, 2 Ar); 7.83 (dd, J 11.1 Hz, 1.7 Hz, 1H, Ar); 8.23 (s, 1H, Ar); 8.44 (d, J 8.3 Hz, 0.35H, one rotamer of Ar); 8.46 (d, J 8.3 Hz, 0.65H, other rotamer of Ar). 1 proton not observed.

M/Z (M[³⁵Cl]+H)⁺: 561.4.

### Compound 413: methyl 6-(1-(tert-butoxycarbonyl)piperidin-4-yl)-2,4-dimethylnicotinate

To a solution of Compound 410 (173 mg) in MeOH (8 mL) was added Pd/C (10% w/w, 26 mg, 0.05 equiv). The reaction mixture was sparged with Hydrogen for 5 min and then stirred at 25° C. for 2 hours under a Hydrogen atmosphere (1 bar). The mixture was filtered through celite and the filtrate was concentrated to dryness. The crude was purified by Flash chromatography (Merck 60®, CyHex 100% to CyHex/EtOAc 84:16) to obtain Compound 413 (115 mg, 66%) as a white solid.

M/Z (M+H)⁺: 349.2.

### Compound 414: methyl 2,4-dimethyl-6-(piperidin-4-yl)nicotinate hydrochloride

Compound 414 was prepared according to general procedure (XVIIIb) starting from Compound 413 (115 mg). The reaction mixture was concentrated under reduced pressure to obtain Compound 414 (110 mg) as a white solid.

M/Z (M+H)⁺: 249.2

### Compound 415: methyl 6-(1-(1-(tert-butyl)-3-(4-chloro-3-fluorophenyl)-1H-pyrrolo[2,3-b]pyridine-6-carbonyl)piperidin-4-yl)-2,4-dimethylnicotinate

Compound 415 was prepared according to general procedure (Xa) starting from Compound 78 (60 mg) and Compound 414 (54 mg, 1.1 equiv), and using N,N-diisopropylethylamine (4.0 equiv). The crude was purified by flash chromatography (Merck 60®, CyHex 100% to CyHex/EtOAc 60:40) to obtain Compound 415 (71 mg) as a white solid.

M/Z (M[³⁵Cl]+H)⁺: 577.4

### Example 168: 6-(1-(1-(tert-butyl)-3-(4-chloro-3-fluorophenyl)-1H-pyrrolo[2,3-b]pyridine-6-carbonyl)piperidin-4-yl)-2,4-dimethylnicotinic acid

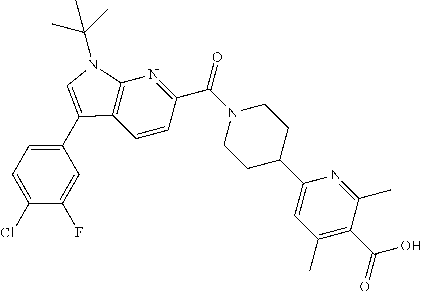 [SMILES: Cc1cc(C2CCN(C(=O)c3ccc4c(-c5ccc(Cl)c(F)c5)cn(C(C)(C)C)c4n3)CC2)nc(C)c1C(=O)O]

Example 168 was prepared according to general procedure (XIc) starting from Compound 415 (170 μmol). The crude was purified by flash chromatography (DCM 100% to DCM/MeOH 80:20) and freeze-dried from EtOH/water to obtain Example 168 (50 mg, 73% over 2 steps) as a white solid.

¹H-NMR (DMSO-d₆, 300 MHz) δ: 1.68-1.75 (m, 2H, CH₂); 1.80 (s, 9H, C(CH₃)₃); 1.83-1.86 (m, 1H, CHₐH_{b}); 1.91-1.99 (m, 1H, CHₐH_{b}); 2.22 (s, 3H, CH₃); 2.39 (s, 3H, CH₃); 2.88-3.00 (m, 2H, CH+N—CHₐH_{b}); 3.15-3.27 (m, 1H, N—CHₐH_{b}); 4.11 (d, J 12.2 Hz, 1H, N—CHₐH_{b}); 4.68 (d, J 12.2 Hz, 1H, N—CHₐH_{b}); 6.96 (s, 1H, Ar); 7.41 (d, J 8.3 Hz, 1H, Ar); 7.59-7.67 (m, 2H, 2 Ar); 7.82 (dd, J 11.1 Hz, 1.7 Hz, 1H, Ar); 8.19 (s, 1H, Ar); 8.43 (d, J 8.3 Hz, 1H, Ar). 1 proton not observed.

M/Z (M[³⁵Cl]+H)⁺: 563.4.

### Compound 416: tert-butyl 4-(5-(methoxycarbonyl)picolinoyl)-2,2-dimethylpiperazine-1-carboxylate

Compound 416 was prepared according to general procedure (Xb) starting from 5-(methoxycarbonyl)picolinic acid (140 mg) and tert-butyl 2,2-dimethylpiperazine-1-carboxylate (182 mg, 1.1 equiv), and using triethylamine (2.0 equiv). The crude was purified by flash chromatography (Merck 60®, CyHex 100% to EtOAc 100%) to obtain Compound 416 (220 mg, 75%) as a white solid.

M/Z (M[³⁵Cl]+H)⁺: 378.3

### Compound 417: methyl 6-(3,3-dimethylpiperazine-1-carbonyl)nicotinate hydrochloride

Compound 417 was prepared according to general procedure (XVIIIc) starting from Compound 416 (220 mg). The reaction mixture was concentrated under reduced pressure to obtain Compound 417 (153 mg, 84%) as a white solid.

M/Z (M+H)⁺: 278.3

### Compound 418: methyl 6-(4-(1-(tert-butyl)-3-(4-chloro-3-fluorophenyl)-1H-pyrrolo[2,3-b]pyridine-6-carbonyl)-3,3-dimethylpiperazine-1-carbonyl)nicotinate

Compound 418 was prepared according to general procedure (Xa) starting from Compound 78 (60 mg) and Compound 417 (60 mg, 1.1 equiv), and using triethylamine (2.0 equiv). The crude was purified by flash chromatography (CyHex 100% to CyHex/EtOAc 50:50) to obtain Compound 418 (67 mg) as a yellow solid.

M/Z (M[³⁵Cl]+H)⁺: 606.5

### Example 169: 6-(4-(1-(tert-butyl)-3-(4-chloro-3-fluorophenyl)-1H-pyrrolo[2,3-b]pyridine-6-carbonyl)-3,3-dimethylpiperazine-1-carbonyl)nicotinic acid

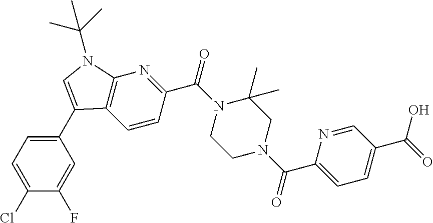 [SMILES: CC1(C)CN(C(=O)c2ccc(C(=O)O)cn2)CCN1C(=O)c1ccc2c(-c3ccc(Cl)c(F)c3)cn(C(C)(C)C)c2n1]

To a solution of Compound 418 (67 mg) in THF (3 mL) and water (15 mL) was added Lithium hydroxide monohydrate (1.5 equiv). The reaction was stirred at 25° C. for 3 hours. The solvent was concentrated to dryness and the residue was solubilized in water (100 mL). 1M aqueous HCl was then added until pH<3. The precipitate was filtered and the solid was washed with water (3×30 mL) to obtain Example 169 (39 mg, 39% over 2 steps) as a white solid.

¹H-NMR (DMSO-d₆, 300 MHz) δ: 1.46 (s, 3.6H, one rotamer of C(CH₃)₃); 1.63 (s, 2.4H, one rotamer of C(CH₃)₂); 1.64 (s, 3.6H, other rotamer of C(CH₃)₂); 1.82 (s, 5.4H, other rotamer of C(CH₃)₃); 3.60-3.66 (m, 2H, N—CH₂); 3.66-3.73 (m, 2H, N—CH₂); 3.82-3.89 (m, 2H, N—CH₂); 7.40 (d, J 8.3 Hz, 0.4H, one rotamer of Ar); 7.41 (d, J 8.3 Hz, 0.6H, other rotamer of Ar); 7.57-7.68 (m, 2H, 2 Ar); 7.74-7.84 (m, 2H, 2 Ar); 8.16 (s, 0.4H, one rotamer of Ar); 8.22 (s, 0.6H, other rotamer of Ar); 8.38-8.45 (m, 2H, 2 Ar); 9.06-9.08 (m, 0.4H, one rotamer of Ar); 9.08-9.10 (m, 0.6H, other rotamer of Ar). 1 proton not observed.

M/Z (M[³⁵Cl]+H)⁺: 592.5.

### Compound 419: methyl 6-(4-(1-isobutyl-3-(6-(trifluoromethyl)pyridin-3-yl)-1H-pyrrolo[2,3-b]pyridine-6-carbonyl)-3,3-dimethylpiperazin-1-yl)-2,4-dimethylnicotinate

Compound 419 was prepared according to general procedure (XVIIa) starting from Compound 340 (200 mg) and (6-(trifluoromethyl)pyridin-3-yl)boronic acid (103 mg, 1.5 equiv). The crude was purified by flash chromatography (CyHex 100% to CyHex/EtOAc 65:35) then by flash chromatography (Interchim®20 μm, CyHex 100% to CyHex/EtOAc 65:35) to obtain Compound 419 (140 mg) as a white solid.

M/Z (M+H)⁺: 623.5

### Example 170: 6-(4-(1-isobutyl-3-(6-(trifluoromethyl)pyridin-3-yl)-1H-pyrrolo[2,3-b]pyridine-6-carbonyl)-3,3-dimethylpiperazin-1-yl)-2,4-dimethylnicotinic acid

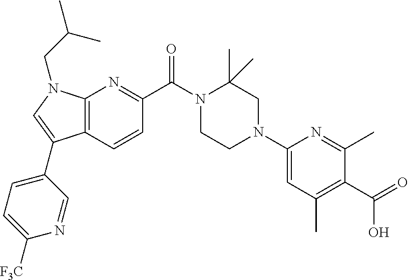 [SMILES: Cc1cc(N2CCN(C(=O)c3ccc4c(-c5ccc(C(F)(F)F)nc5)cn(CC(C)C)c4n3)C(C)(C)C2)nc(C)c1C(=O)O]

Example 170 was prepared according to general procedure (XIb) starting from Compound 419 (140 mg). The crude was purified by flash chromatography (DCM 100% to DCM/MeOH 80:20) and freeze-dried with EtOH/water to obtain Example 170 (72 mg, 52%) as a beige solid.

¹H-NMR (DMSO-d₆, 300 MHz) δ:0.93 (d, J 6.6 Hz, 6H, 2*CH₃); 1.54 (s, 6H, 2*CH₃); 2.24-2.33 (m, 4H, CH, CH₃); 2.38 (s, 3H, CH₃); 3.54-3.62 (m, 2H, CH₂); 3.78-3.85 (m, 2H, CH₂); 3.91 (s, 2H, CH₂); 4.18 (d, 2H, J 7.2, CH₂); 6.32 (s, 1H, Ar); 7.46 (d, 1H, J 8.1 Hz, Ar); 7.94 (d, 1H, J 8.1 Hz, Ar); 8.43 (dd, 1H, J 8.4, 1.5 Hz, Ar), 8.47 (s, 1H, Ar), 8.54 (d, 1H, J 8.1 Hz, Ar); 9.18 (d, 1H, J 1.5 Hz, Ar). 1 proton not observed.

M/Z (M+H)⁺: 609.5.

### Compound 420: 6-chloro-3-(4-chloro-3-fluorophenyl)-1-(cyclobutylmethyl)-1H-pyrrolo[2,3-b]pyridine

Compound 420 was prepared according to general procedure (XIII) in DMA starting from Compound 9 (150 mg) and (bromomethyl)cyclobutene (103 mg, 1.5 equiv). The crude was purified by flash chromatography (CyHex 100% to CyHex/EtOAc 90:10) to obtain Compound 420 (125 mg, 67%) as a white solid.

M/Z (M[³⁵Cl]₂+H)⁺: 349.0

### Compound 421: methyl 6-(4-(3-(4-chloro-3-fluorophenyl)-1-(cyclobutylmethyl)-1H-pyrrolo[2,3-b]pyridine-6-carbonyl)-3,3-dimethylpiperazin-1-yl)-2,4-dimethylnicotinate

Compound 421 was prepared according to general procedure (XXX) starting from Compound 420 (125 mg) and Compound 95 (225 mg, 2.0 equiv). The crude was purified by flash chromatography (CyHex 100% to CyHex/EtOAc 50:50) to obtain Compound 421 (128 mg, 58%) as a white solid.

M/Z (M[³⁵Cl]+H)⁺: 618.5

### Example 171: 6-(4-(3-(4-chloro-3-fluorophenyl)-1-(cyclobutylmethyl)-1H-pyrrolo[2,3-b]pyridine-6-carbonyl)-3,3-dimethylpiperazin-1-yl)-2,4-dimethylnicotinic acid

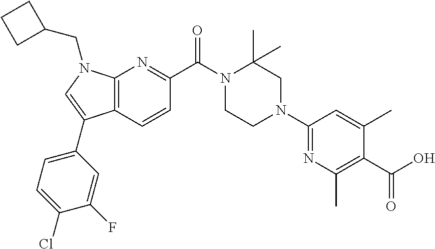 [SMILES: Cc1cc(N2CCN(C(=O)c3ccc4c(-c5ccc(Cl)c(F)c5)cn(CC5CCC5)c4n3)C(C)(C)C2)nc(C)c1C(=O)O]

Example 171 was prepared according to general procedure (XIb) starting from Compound 421 (128 mg). The crude was purified by preparative HPLC (40% to 80% ACN/H₂O), then freeze-dried with water to obtain Example 171 (28 mg, 22%) as a white solid.

¹H-NMR (DMSO-d₆, 300 MHz) δ: 1.54 (s, 6H, 2 CH₃); 1.81-1.94 (m, 4H, 2 CH₂ of CyBu); 1.98-2.05 (m, 2H, CH₂ of CyBu); 2.27 (s, 3H, CH₃—Ar); 2.39 (s, 3H, CH₃—Ar); 2.86-2.91 (m, 1H, CH of CyBu); 3.56-3.60 (m, 2H, CH₂); 3.79-3.83 (m, 2H, CH₂); 3.92 (bs, 2H, CH₂); 4.36 (d, J 7.3 Hz, 2H, CH₂—Ar); 6.34 (s, 1H, Ar); 7.41 (d, J 8.2 Hz, 1H, Ar); 7.63-7.64 (m, 2H, Ar); 7.76-7.79 (m, 1H, Ar); 8.28 (s, 1H, Ar); 8.46 (d, J 8.2 Hz, 1H, Ar); 1 proton not observed.

M/Z (M[³⁵Cl]+H)⁺: 604.5.

### Compound 422: (S)-1-(sec-butyl)-6-chloro-3-(4-chloro-3-fluorophenyl)-1H-pyrrolo[2,3-b]pyridine

Compound 422 was prepared according to general procedure (IV) starting from Compound 3 (500 mg) and (S)-butan-2-amine (287 mg, 2.5 equiv). The crude was purified by flash chromatography (CyHex 100% to CyHex/EtOAc 90:10) to obtain Compound 422 (336 mg, 63%) as a yellow solid.

M/Z (M[³⁵Cl₂]+H)⁺: 337.6

### Compound 423: (S)-1-(sec-butyl)-3-(4-chloro-3-fluorophenyl)-1H-pyrrolo[2,3-b]pyridine-6-carbonitrile

Compound 423 was prepared according to general procedure (V) starting from Compound 422 (336 mg). The crude was purified by flash chromatography (CyHex 100% to CyHex/EtOAc 90:10) to obtain Compound 423 (274 mg, 84%) as a yellow solid.

M/Z (M[³⁵Cl]+H)⁺: 328.7

### Compound 424: (S)-1-(sec-butyl)-3-(4-chloro-3-fluorophenyl)-1H-pyrrolo[2,3-b]pyridine-6-carboxamide

Compound 424 was obtained (532 mg), as a yellow solid according to general procedure (VII) starting from Compound 423 (274 mg).

M/Z (M[³⁵Cl]+H)⁺: 346.1

### Compound 425: methyl (S)-1-(sec-butyl)-3-(4-chloro-3-fluorophenyl)-1H-pyrrolo[2,3-b]pyridine-6-carboxylate

Compound 425 prepared according to general procedure (VIII) starting from Compound 424 (836 μmol). The crude was purified by flash chromatography (CyHex 100% to CyHex/EtOAc 80:20) to obtain Compound 425 (172 mg, 57% over 2 steps) as a white solid.

M/Z (M[³⁵Cl]+H)⁺: 361.2

### Compound 426: (S)-1-(sec-butyl)-3-(4-chloro-3-fluorophenyl)-1H-pyrrolo[2,3-b]pyridine-6-carboxylic acid

Compound 426 was prepared according to general procedure (IXa) starting from Compound 425 (172 mg) to obtain Compound 426 (200 mg) as a white solid.

M/Z (M[³⁵Cl]+H)⁺: 347.1

### Compound 427: methyl (S)-6-(4-(1-(sec-butyl)-3-(4-chloro-3-fluorophenyl)-1H-pyrrolo[2,3-b]pyridine-6-carbonyl)-3,3-dimethylpiperazin-1-yl)-2,4-dimethylnicotinate

Compound 427 was prepared according to general procedure (Xa) starting from Compound 426 (100 mg) and Compound 95 (100 mg, 1.1 equiv), and using N,N-diisopropylethylamine (4.0 equiv). The crude was purified by flash chromatography (CyHex 100% to CyHex/EtOAc 60:40) to obtain Compound 427 (112 mg) as a clear oil.

M/Z (M[³⁵Cl]+H)⁺: 606.5

### Example 172: (S)-6-(4-(1-(sec-butyl)-3-(4-chloro-3-fluorophenyl)-1H-pyrrolo[2,3-b]pyridine-6-carbonyl)-3,3-dimethylpiperazin-1-yl)-2,4-dimethylnicotinic acid

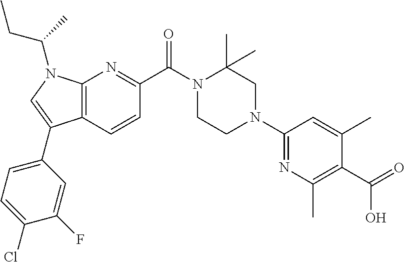 [SMILES: CC[C@H](C)n1cc(-c2ccc(Cl)c(F)c2)c2ccc(C(=O)N3CCN(c4cc(C)c(C(=O)O)c(C)n4)CC3(C)C)nc21]

Example 172 was prepared according to general procedure (XIb) starting from Compound 427 (112 mg). The crude was purified by flash chromatography (CyHex 100% to CyHex/EtOAc 60:40) then triturated in MeOH and freeze dried from EtOH/water (1:9) to obtain Example 172 (14 mg, 8% over 2 steps) as a white solid.

¹H-NMR (DMSO-d₆, 300 MHz) δ: 0.76 (t, J 7.3 Hz, N—CH(CH₃)—CH₂—CH₃); 1.53-1.55 (m, 9H, 2 CH₃+N—CH(CH₃)—CH₂—CH₃); 1.87-1.99 (m, 2H, N—CH(CH₃)—CH₂—CH₃); 2.29 (s, 3H, CH₃); 2.42 (s, 3H, CH₃); 3.56-3.60 (m, 2H, N—CH₂); 3.78-3.83 (m, 2H, N—CH₂); 3.93 (s, 2H, N—CH₂); 4.89-4.96 (m, 1H, N—CH(CH₃)—CH₂—CH₃); 6.45 (s, 1H, Ar); 7.40 (d, J 8.2 Hz, 1H, Ar); 7.61-7.69 (m, 2H, 2 Ar); 7.82 (dd, J 11.1 Hz, 1.7 Hz, 1H, Ar); 8.40 (s, 1H, Ar); 8.47 (d, J 8.2 Hz, 1H, Ar); 1 proton not observed.

M/Z (M[³⁵Cl]+H)⁺: 592.5.

### Compound 428: 2-((6-chloro-3-(4-chloro-3-fluorophenyl)-1H-pyrrolo[2,3-b]pyridin-1-yl)methyl)oxazole

Compound 428 was prepared according to general procedure (XIII) in DMA starting from Compound 9 (200 mg) and 2-(chloromethyl)oxazole (109 mg, 1.3 equiv). The crude was purified by flash chromatography (CyHex 100% to CyHex/EtOAc 50:50) to obtain Compound 428 (102 mg, 40%) as a white solid.

M/Z (M[³⁵Cl]₂+H)⁺: 362.1

### Compound 429: methyl 6-(4-(3-(4-chloro-3-fluorophenyl)-1-(oxazol-2-ylmethyl)-1H-pyrrolo[2,3-b]pyridine-6-carbonyl)-3,3-dimethylpiperazin-1-yl)-2,4-dimethylnicotinate

Compound 429 was prepared according to general procedure (XXX) starting from Compound 428 (102 mg) and Compound 95 (176 mg, 2.0 equiv). The crude was purified by flash chromatography (CyHex 100% to CyHex/EtOAc 30:70) to obtain Compound 429 (96 mg, 54%) as a yellow solid.

M/Z (M[³⁵Cl]+H)⁺: 631.4

### Example 173: 6-(4-(3-(4-chloro-3-fluorophenyl)-1-(cyclobutylmethyl)-1H-pyrrolo[2,3-b]pyridine-6-carbonyl)-3,3-dimethylpiperazin-1-yl)-2,4-dimethylnicotinic acid

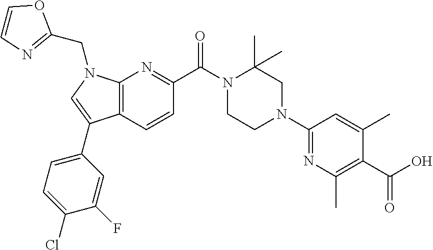 [SMILES: Cc1cc(N2CCN(C(=O)c3ccc4c(-c5ccc(Cl)c(F)c5)cn(Cc5ncco5)c4n3)C(C)(C)C2)nc(C)c1C(=O)O]

Example 173 was prepared according to general procedure (XIb) starting from Compound 429 (96 mg). The crude was purified by preparative HPLC (30% to 90% ACN/H₂O), then freeze-dried with water to obtain Example 173 (20 mg, 21%) as a white solid.

¹H-NMR (DMSO-d₆, 300 MHz) δ: 1.51 (s, 6H, 2 CH₃); 2.28 (s, 3H, CH₃—Ar); 2.39 (s, 3H, CH₃—Ar); 3.43 (t, J 5.1 Hz, 2H, CH₂); 3.69 (t, J 5.1 Hz, 2H, CH₂); 3.90 (s, 2H, CH₂); 5.74 (s, 2H, Ar—CH₂—Ar); 6.30 (s, 1H, Ar); 7.20 (d, J 0.8 Hz, 1H, Ar); 7.46 (d, J 8.2 Hz, 1H, Ar); 7.61-7.69 (m, 2H, Ar); 7.76-7.80 (m, 1H, Ar); 8.10 (d, J 0.8 Hz, 1H, Ar); 8.33 (s, 1H, Ar); 8.50 (d, J 8.2 Hz, 1H, Ar); 12.67 (s, 1H, COOH).

M/Z (M[³⁵Cl]+H)⁺: 617.4.

### Compound 430: 5-methyl-1H-pyrrolo[2,3-b]pyridine 7-oxide hydrochloride

Compound 430 was obtained (1.25 g) as a brown solid according to general procedure (XIXa) from 5-methyl-1H-pyrrolo[2,3-b]pyridine (0.98 g) and 3-chlorobenzoperoxoic acid<77% (2.99 g, 1.8 equiv).

M/Z (M+H)⁺: 149.0

### Compound 431: 5-methyl-1H-pyrrolo[2,3-b]pyridine-6-carbonitrile

Compound 431 was prepared according to general procedure (XX) from Compound 430 (7.41 mmol). The crude was purified by flash chromatography (CyHex 100% to CyHex/EtOAc 80:20) to obtain Compound 431 (560 mg, 53% over 2 steps) as a yellow solid.

M/Z (M+H)⁺: 158.5

### Compound 432: 3-bromo-5-methyl-1H-pyrrolo[2,3-b]pyridine-6-carbonitrile

Compound 432 was prepared according to general procedure (XIV) from Compound 431 (450 mg) and purified by flash chromatography (Merck 60®, CyHex 100% to EtOAc 100%) to afford Compound 432 (650 mg, 96%) as a salmon solid.

M/Z (M[⁷⁹Br]+H)⁺: 236.0

### Compound 433: 3-bromo-1-isobutyl-5-methyl-1H-pyrrolo[2,3-b]pyridine-6-carbonitrile

Compound 433 was prepared according to general procedure (XIII) in DMA from Compound 432 (650 mg) and isobutyl iodide (389 μL, 1.3 equiv). The reaction mixture was stirred for 25 hours. The crude was purified by flash chromatography (CyHex 100% to CyHex/EtOAc 80:20) to obtain Compound 433 (622 mg, 77%) as a white solid.

M/Z (M[⁸¹Br]+H)⁺: 294.2

### Compound 434: 3-bromo-1-isobutyl-5-methyl-1H-pyrrolo[2,3-b]pyridine-6-carboxamide

Compound 434 was obtained (600 mg, 82%) as a white solid according to general procedure (VII) starting from Compound 433 (690 mg).

M/Z (M[⁷⁹Br]+H)⁺: 312.2

### Compound 435: 3-(4-chloro-3-fluorophenyl)-1-isobutyl-5-methyl-1H-pyrrolo[2,3-b]pyridine-6-carboxamide

Compound 435 was prepared according to general procedure (XVIIa) starting from Compound 434 (483 mg) (4-chloro-3-fluorophenyl)boronic acid (326 mg, 1.2 equiv). The crude was purified by flash chromatography (CyHex 100% to EtOAc 100%) to obtain Compound 435 (545 mg) as a yellow solid.

M/Z (M[³⁵Cl]+H)⁺: 360.2

### Compound 436: methyl 3-(4-chloro-3-fluorophenyl)-1-isobutyl-5-methyl-1H-pyrrolo[2,3-b]pyridine-6-carboxylate

Compound 436 was prepared according to general procedure (VIII) starting from Compound 435 (1.56 mmol). The crude was purified by flash chromatography (Merck 60®, CyHex 100% to CyHex/EtOAc 60:40) to obtain Compound 436 (423 mg, 72% over 2 steps) as a white solid.

M/Z (M[³⁵Cl]+H)⁺: 375.2

### Compound 437: 3-(4-chloro-3-fluorophenyl)-1-isobutyl-5-methyl-1H-pyrrolo[2,3-b]pyridine-6-carboxylic acid

Compound 437 was obtained (352 mg, 87%) as a brown solid according to general procedure (IXa) starting from Compound 436 (420 mg) M/Z (M[³⁵Cl]+H)⁺: 361.3

### Compound 438: methyl 6-(4-(3-(4-chloro-3-fluorophenyl)-1-isobutyl-5-methyl-1H-pyrrolo[2,3-b]pyridine-6-carbonyl)-3,3-dimethylpiperazin-1-yl)-2,4-dimethylnicotinate

Compound 438 was prepared according to general procedure (Xa) starting from Compound 437 (60 mg) and Compound 95 (57 mg, 1.1 equiv), and using N,N-diisopropylethylamine (4.0 equiv). The crude was purified by flash chromatography (Merck 60®, CyHex 100% to EtOAc 100%) to obtain Compound 438 (90 mg) as a white solid.

M/Z (M[³⁵Cl]+H)⁺: 620.5

### Example 174: 6-(4-(3-(4-chloro-3-fluorophenyl)-1-isobutyl-5-methyl-1H-pyrrolo[2,3-b]pyridine-6-carbonyl)-3,3-dimethylpiperazin-1-yl)-2,4-dimethylnicotinic acid

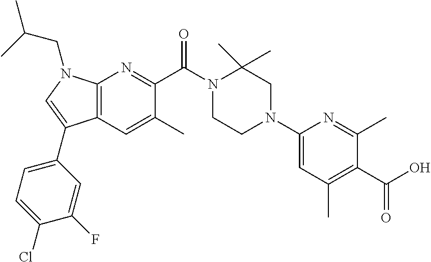 [SMILES: Cc1cc2c(-c3ccc(Cl)c(F)c3)cn(CC(C)C)c2nc1C(=O)N1CCN(c2cc(C)c(C(=O)O)c(C)n2)CC1(C)C]

Example 174 was prepared according to general procedure (XIb) starting from Compound 438 (90 mg). The crude was purified by preparative HPLC (Column B, 20% to 100% ACN/H₂O), then freeze-dried with water to obtain Example 174 (45 mg, 51%) as a white solid.

¹H-NMR (DMSO-d₆, 300 MHz) δ: 0.85 (d, J 6.6 Hz, 6H, (CH₃)₂); 1.57 (s, 6H, (CH₃)₂); 2.40-2.48 (m, 4H, CH₃+CH); 2.36 (s, 3H, CH₃); 2.38 (s, 3H, CH₃); 3.39-3.44 (m, 4H, 2×CH₂); 3.91 (s, 2H, CH₂); 4.08 (d, J 6.6 Hz, 2H, CH₂); 6.29 (s, 1H, Ar); 7.58-7.62 (m, 2H, Ar); 7.74-7.78 (m, 1H, Ar); 8.16 (s, 1H, Ar); 8.28 (s, 1H, Ar); 12.61 (bs, 1H, CO₂H).

M/Z (M[³⁵Cl]+H)⁺: 606.6.

### Compound 439: 7H-pyrrolo[2,3-d]pyrimidine-2-carbonitrile

Compound 439 was prepared according to general procedure (Vb) from 2-chloro-7H-pyrrolo[2,3-d]pyrimidine (500 mg). The crude was purified by flash chromatography (CyHex 100% to CyHex/EtOAc 50:50) to obtain Compound 439 (205 mg, 44%) as a white solid.

M/Z (M+H)⁺: 145.0

### Compound 440: 5-bromo-7H-pyrrolo[2,3-d]pyrimidine-2-carbonitrile

Compound 440 was prepared according to general procedure (XIV) from Compound 439 (205 mg) and purified by flash chromatography (CyHex 100% to CyHex/EtOAc 70:30) to afford Compound 440 (218 mg, 69%) as a white solid.

M/Z (M[⁸¹Br]+H)⁺: 224.9.

### Compound 441: 5-bromo-7-isobutyl-7H-pyrrolo[2,3-d]pyrimidine-2-carbonitrile

Compound 441 was prepared according to general procedure (XIII) in NMP from Compound 440 (218 mg) and isobutyl iodide (220 μL, 2 equiv). The reaction mixture was stirred for 17 hours at 50° C. The crude was purified by flash chromatography (CyHex 100% to CyHex/EtOAc 70:30) to obtain Compound 441 (257 mg, 94%) as a white solid.

M/Z (M[⁸¹Br]+H)⁺: 279.0

### Compound 442: 5-(4-chloro-3-fluorophenyl)-7-isobutyl-7H-pyrrolo[2,3-d]pyrimidine-2-carbonitrile

Compound 442 was prepared according to general procedure (XVIIa) starting from Compound 441 (232 mg) (4-chloro-3-fluorophenyl)boronic acid (174 mg, 1.2 equiv). The crude was purified by flash chromatography (CyHex 100% to CyHex/EtOAc 70:30) to obtain Compound 442 (241 mg, 80%) as a beige solid.

M/Z (M[³⁵Cl]+H)⁺: 329.1

### Compound 443: 5-(4-chloro-3-fluorophenyl)-7-isobutyl-7H-pyrrolo[2,3-d]pyrimidine-2-carboxamide

Compound 443 was obtained (217 mg) as a white solid according to general procedure (VII) starting from Compound 442 (241 mg).

M/Z (M[³⁵Cl]+H)⁺: 347.1

### Compound 444: methyl 5-(4-chloro-3-fluorophenyl)-7-isobutyl-7H-pyrrolo[2,3-d]pyrimidine-2-carboxylate

Compound 444 was prepared according to general procedure (VIII) starting from Compound 443 (0.73 mmol). The crude was purified by flash chromatography (CyHex 100% to CyHex/EtOAc 10:90) to obtain Compound 444 (75 mg, 28% over 2 steps) as a white solid.

M/Z (M[³⁵Cl]+H)⁺: 362.2

### Compound 445: 5-(4-chloro-3-fluorophenyl)-7-isobutyl-7H-pyrrolo[2,3-d]pyrimidine-2-carboxylic acid

Compound 445 was obtained (54 mg, 76%) as a white solid according to general procedure (IXa) starting from Compound 444 (74 mg) M/Z (M[³⁵Cl]+H)⁺: 348.1

### Compound 446: methyl 6-(4-(5-(4-chloro-3-fluorophenyl)-7-isobutyl-7H-pyrrolo[2,3-d]pyrimidine-2-carbonyl)-3,3-dimethylpiperazin-1-yl)-2,4-dimethylnicotinate

Compound 446 was prepared according to general procedure (Xa) starting from Compound 445 (54 mg) and Compound 95 (54 mg, 1.1 equiv), and using N,N-diisopropylethylamine (4.0 equiv). The crude was purified by flash chromatography (CyHex 100% to CyHex/EtOAc 40:60) to obtain Compound 446 (89 mg, 94%) as a white solid.

M/Z (M[³⁵Cl]+H)⁺: 607.5

### Example 175: 6-(4-(5-(4-chloro-3-fluorophenyl)-7-isobutyl-7H-pyrrolo[2,3-d]pyrimidine-2-carbonyl)-3,3-dimethylpiperazin-1-yl)-2,4-dimethylnicotinic acid

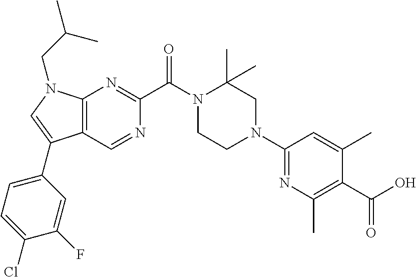 [SMILES: Cc1cc(N2CCN(C(=O)c3ncc4c(-c5ccc(Cl)c(F)c5)cn(CC(C)C)c4n3)C(C)(C)C2)nc(C)c1C(=O)O]

Example 175 was prepared according to general procedure (XIb) starting from Compound 446 (64 mg). The crude was purified by preparative HPLC (30% to 70% ACN/H₂O), then freeze-dried with water to obtain Example 175 (18 mg, 28%) as a white solid.

¹H-NMR (DMSO-d₆, 300 MHz) δ: 0.9 (d, J 6.7 Hz, 6H, CH₃—CH—CH₃); 1.56 (s, 6H, 2 CH₃); 2.22-2.32 (m, 1H, CH₃—CH—CH₃); 2.27 (s, 3H, CH₃—Ar); 2.39 (s, 3H, CH₃—Ar); 3.46-3.49 (m, 4H, 2 CH₂); 3.92 (bs, 2H, CH₂); 4.13 (d, J 7.2 Hz, 2H, CH₂—Ar); 6.35 (s, 1H, Ar); 7.64-7.69 (m, 1H, Ar); 7.72 (d, J 8.4, 2.0 Hz, 1H, Ar); 7.87 (d, J 10.9, 1.7 Hz, 1H, Ar); 8.34 (s, 1H, Ar); 9.44 (s, 1H, Ar); 12.70 (bs, 1H, CO₂H).

M/Z (M[³⁵Cl]+H)⁺: 593.4

### Compound 447: 2-(2-chloropyridin-4-yl)-2-methylpropanenitrile

At −78° C., to a solution of isobutyronitrile (394 mg, 1.5 equiv) in THF (19 mL) was added dropwise LiHMDS (4.9 mL, 1.0 molar in THF, 1.3 eq., 4.94 mmol). The reaction was stirred at −78° C. for 30 min, then a solution of 2-Chloro-4-fluoropyridine (500 mg) in THF (6 mL) was added dropwise. The reaction was stirred at −78° C. for 30 min, then let warm up at room temperature for 18 hours. The reaction mixture was quenched with NH₄Cl sat. (40 mL), extracted with EtOAc (40 mL), washed with brine (40 mL), dried over magnesium sulfate then concentrated to dryness to obtain Compound 447 (610 mg, 89%) as a yellow oil.

M/Z (M[³⁵Cl]+H)⁺: 181.1

### Compound 448: tert-butyl 4-(4-(2-cyanopropan-2-yl)pyridin-2-yl)-2,2-dimethylpiperazine-1-carboxylate

Compound 448 was prepared according to general procedure (XXI) starting from Compound 447 (130 mg) and tert-butyl 2,2-dimethylpiperazine-1-carboxylate (185 mg, 1.2 Eq). The crude was purified by flash chromatography (Merck 60®, CyHex 100% to CyHex/EtOAc 50:50) to obtain Compound 448 as a white solid (130 mg, 50%).

M/Z (M+H)⁺: 359.3

### Compound 449: 2-(2-(3,3-dimethylpiperazin-1-yl)pyridin-4-yl)-2-methylpropanenitrile hydrochloride

Compound 449 was prepared according to general procedure (XVIIIc) starting from Compound 448 (130 mg). The reaction mixture was concentrated under reduced pressure and triturated in Et₂O (20 mL) to obtain Compound 449 (83 mg, 78%) as a white solid.

M/Z (M+H)⁺: 259.3

### Example 176: 2-(2-(4-(1-(tert-butyl)-3-(4-chloro-3-fluorophenyl)-1H-pyrrolo[2,3-b]pyridine-6-carbonyl)-3,3-dimethylpiperazin-1-yl)pyridin-4-yl)-2-methylpropanenitrile

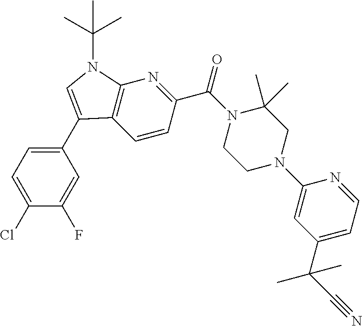 [SMILES: CC(C)(C#N)c1ccnc(N2CCN(C(=O)c3ccc4c(-c5ccc(Cl)c(F)c5)cn(C(C)(C)C)c4n3)C(C)(C)C2)c1]

Example 176 was prepared according to general procedure (Xa) starting from Compound 78 (89 mg) and Compound 449 (83 mg, 1.1 equiv), and using triethylamine (4.0 equiv). The crude was purified by flash chromatography (CyHex 100% to CyHex/EtOAc 60:40) then freeze dried from EtOH/water to obtain Example 176 (120 mg, 80%) as a white solid.

¹H-NMR (DMSO-d₆, 300 MHz) δ: 1.56 (s, 6H, C(CH₃)₂); 1.67 (s, 6H, N—C(CH₃)₂); 1.82 (s, 9H, C(CH₃)₃); 3.59 (t, 2H, J 5.2 Hz, 2H, CH₂); 3.90 (t, 2H, J 5.2 Hz, 2H, CH₂); 3.96 (s, 2H, CH₂); 6.62 (s, 1H, Ar); 6.74 (dd, J 5.2 Hz, 1.4 Hz, 1H, Ar); 7.43 (d, J 8.3 Hz, 1H, Ar); 7.60-7.68 (m, 2H, 2 Ar); 7.83 (dd, J 11.1 Hz, 1.7 Hz, 1H, Ar); 8.13 (d, J 5.2 Hz, 1H, Ar); 8.21 (s, 1H, Ar); 8.44 (d, J 8.3 Hz, 1H, Ar).

M/Z (M[³⁵Cl]+H)⁺: 587.5

### Example 177: 2-(2-(4-(1-(tert-butyl)-3-(4-chloro-3-fluorophenyl)-1H-pyrrolo[2,3-b]pyridine-6-carbonyl)-3,3-dimethylpiperazin-1-yl)pyridin-4-yl)-2-methylpropanamide

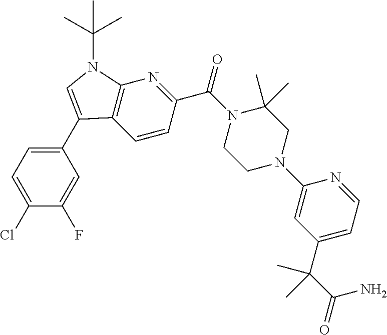 [SMILES: CC(C)(C(N)=O)c1ccnc(N2CCN(C(=O)c3ccc4c(-c5ccc(Cl)c(F)c5)cn(C(C)(C)C)c4n3)C(C)(C)C2)c1]

Example 177 was prepared according to general procedure (VII) starting from Example 176 (100 mg). The crude was purified by flash chromatography (Interchim®20 μm, CyHex 100% to EtOAc 100%) then freeze dried from EtOH/water to obtain Example 177 (53 mg, 51%) as a white solid

¹H-NMR (DMSO-d₆, 300 MHz) δ: 1.41 (s, 6H, (CH₃)₂); 1.56 (s, 6H, C(CH₃)₂); 1.83 (s, 9H, C(CH₃)₃); 3.54 (t, J 5.2 Hz, 2H, CH₂); 3.88 (t, J 5.2 Hz, 2H, CH₂); 3.92 (s, 2H, CH₂); 6.47 (s, 1H, Ar); 6.56 (d, J 5.2 Hz, 1H, Ar); 6.88-7.05 (m, 2H, NH₂); 7.42 (d, J 8.3 Hz, 1H, Ar); 7.60-7.68 (m, 2H, 2 Ar); 7.82 (dd, J 11.1 Hz, 1.7 Hz, 1H, Ar); 8.01 (d, J 5.2 Hz, 1H, Ar); 8.21 (s, 1H, Ar); 8.43 (d, J 8.3 Hz, 1H, Ar).

M/Z (M[³⁵Cl]+H)⁺: 605.6.

### Compound 450: tert-butyl 4-(5-bromopyridin-2-yl)-2,2-dimethylpiperazine-1-carboxylate

To a solution of 5-bromo-2-fluoropyridine (100 mg, 1.0 equiv) in Toluene (1 mL) was added Tert-butyl 22-dimethylpiperazine-1-carboxylate (487 mg, 4.0 equiv). The reaction was heated at 125° C. for 3 hours. The reaction mixture was concentrated to dryness. H₂O (50 mL) was added and the aqueous layer was extracted with EtOAc (3×50 mL). The combined organic layers were washed with brine (50 mL), dried over magnesium sulfate then concentrated to dryness. The crude was purified by flash chromatography (CyHex 100% to CyHex/EtOAc 70:30) to obtain compound 450 (110 mg, 52%) as a white solid.

M/Z (M[⁸¹Br]+H)⁺: 372.1.

### Compound 451: tert-butyl 4-(5-(diethoxyphosphoryl)pyridin-2-yl)-2,2-dimethylpiperazine-1-carboxylate

To a solution of Diethyl phosphite (49 mg, 1.2 equiv) in DMF (1 mL) were added Compound 450 (110 mg, 1 equiv) and DIPEA (67 μL, 1.3 equiv). The mixture was sparged with Argon for 10 minutes then Palladium diacetate (6.67 mg, 0.1 equiv) and 1,1′-Ferrocendiylbis(diphenylphosphine) (18 mg, 0.11 equiv) were added. The reaction was heated at 110° C. for 20 hours. The reaction mixture was concentrated to dryness. H₂O (50 mL) was added and the aqueous layer was extracted with EtOAc (3×50 mL). The combined organic layers (EtOAc) were washed with brine (50 mL), dried over magnesium sulfate then concentrated to dryness. The crude was purified by flash chromatography (CyHex 100% to CyHex/EtOAc 10:90) to obtain Compound 451 (90 mg) as a white solid.

M/Z (M+H)⁺: 428.3.

### Compound 452: diethyl (6-(3,3-dimethylpiperazin-1-yl)pyridin-3-yl)phosphonate hydrochloride

Compound 452 was prepared according to general procedure (XVIIIc) starting from Compound 451 (130 mg). The reaction mixture was concentrated under reduced pressure and triturated in Et₂O (20 mL) to obtain Compound 452 (80 mg) as a yellow solid.

M/Z (M+H)⁺: 328.3

### Example 178: diethyl (6-(4-(1-(tert-butyl)-3-(4-chloro-3-fluorophenyl)-1H-pyrrolo[2,3-b]pyridine-6-carbonyl)-3,3-dimethylpiperazin-1-yl)pyridin-3-yl)phosphonate

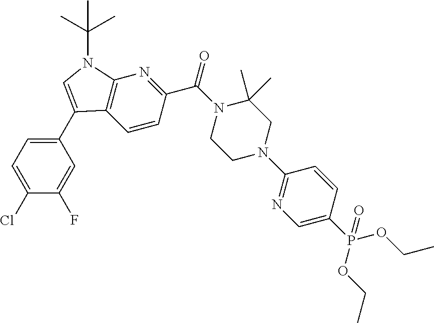 [SMILES: CCOP(=O)(OCC)c1ccc(N2CCN(C(=O)c3ccc4c(-c5ccc(Cl)c(F)c5)cn(C(C)(C)C)c4n3)C(C)(C)C2)nc1]

Example 178 was prepared according to general procedure (Xa) starting from Compound 78 (67 mg) and Compound 452 (80 mg, 1.1 equiv) and using N,N-diisopropylethylamine (4.0 equiv). The crude was purified by flash chromatography (CyHex 100% to CyHex/EtOAc 20:80) then further purified by flash chromatography (Interchim® 20 μm, CyHex 100% to CyHex/EtOAc 20:80) and freeze dried with EtOH/water to obtain Example 178 (69 mg, 54%) as a white solid.

¹H-NMR (DMSO-d₆, 300 MHz) δ: 1.22 (t, J 7.1 Hz, 6H, PO—CH₂—CH₃)₂); 1.57 (s, 6H, C(CH₃)₂); 1.82 (s, 9H, C(CH₃)₃); 3.58 (t, J 5.2 Hz, 2H, CH₂); 3.90 (t, J 5.2 Hz, 2H, CH₂); 3.93-4.06 (m, 6H, PO—CH₂—CH₃)₂)+N—CH₂); 6.71 (dd, J 8.3 Hz, 1.7 Hz, 1H, Ar); 7.41 (d, J 8.3 Hz, 1H, Ar); 7.60-7.68 (m, 2H, 2 Ar); 7.69-7.78 (m, 1H, Ar); 7.82 (dd, J 11.1 Hz, 1.7 Hz, 1H, Ar); 8.21 (s, 1H, Ar); 8.35 (dd, J 6.4 Hz, 2.1 Hz, 1H, Ar); 8.43 (d, J 8.3 Hz, 1H, Ar).

M/Z (M[³⁵Cl]+H)⁺: 656.4.

### Compound 453: tert-butyl (S)-4-(5-(methoxycarbonyl)-4,6-dimethylpyridin-2-yl)-2-methylpiperazine-1-carboxylate

Compound 453 was prepared according to general procedure (XXI) starting from methyl 6-chloro-2,4-dimethylnicotinate (272 mg) and tert-butyl (S)-2-methylpiperazine-1-carboxylate (300 mg). The crude residue was purified by flash chromatography (Merck 60®, CyHex 100% to CyHex/EtOAc 50:50) to obtain Compound 453 (400 mg) as a white solid.

M/Z (M+H)⁺: 350.2

### Compound 454: methyl (S)-2,4-dimethyl-6-(3-methylpiperazin-1-yl)nicotinate hydrochloride

Compound 454 was obtained (300 mg) as a white solid according to general procedure (XVIIIc) starting from Compound 453 (400 mg).

M/Z (M+H)⁺: 264.2

### Compound 455: methyl (S)-6-(4-(1-(tert-butyl)-3-(4-chloro-3-fluorophenyl)-1H-pyrrolo[2,3-b]pyridine-6-carbonyl)-3-methylpiperazin-1-yl)-2,4-dimethylnicotinate

Compound 455 was prepared according to general procedure (Xb) starting from Compound 78 (86 mg), Compound 454 (110 mg) and N,N-diisopropylethylamine (4.0 equiv). The crude residue was purified by flash chromatography (CyHex 100% to CyHex/EtOAc 0:100) to obtain Compound 455 (145 mg) as a white solid.

### Example 179: (S)-6-(4-(1-(tert-butyl)-3-(4-chloro-3-fluorophenyl)-1H-pyrrolo[2,3-b]pyridine-6-carbonyl)-3-methylpiperazin-1-yl)-2,4-dimethyinicotinic acid

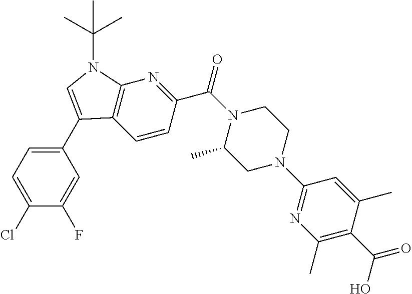 [SMILES: Cc1cc(N2CCN(C(=O)c3ccc4c(-c5ccc(Cl)c(F)c5)cn(C(C)(C)C)c4n3)[C@@H](C)C2)nc(C)c1C(=O)O]

Example 179 was prepared according to general procedure (XIb) starting from Compound 455 (145 mg). The crude was purified by flash chromatography (DCM 100% to DCM/MeOH 80:20), then freeze-dried in MeOH/water to obtain Example 179 (128 mg, 48% over 4 steps) as a beige solid.

¹H-NMR (DMSO-d₆, 300 MHz) δ: 1.24 (d, J 6.6 Hz, 3H, CH₃); 1.81 (s, 9H, C(CH₃)₃); 2.23 (s, 3H, CH₃); 2.34 (s, 3H, CH₃); 2.85-3.08 (m, 2H, CH₂); 3.94-4.86 (m, 5H, CH, 2*CH₂); 6.54 (s, 1H, Ar); 7.45 (d, 1H, J 8.1 Hz, Ar); 7.59-7.68 (m, 2H, Ar); 7.82 (dd, 1H, J 9.9, 1.8 Hz, Ar); 8.22 (s, 1H, Ar); 8.44 (d, 1H, J 8.1 Hz, Ar). 1 proton not observed.

M/Z (M[³⁵Cl]+H)⁺: 578.3

### Compound 456: methyl 2-(6-chloro-3-(4-chloro-3-fluorophenyl)-1H-pyrrolo[2,3-b]pyridin-1-yl)acetate

Compound 456 was prepared according to general procedure (XIII) in DMA starting from Compound 9 (557 mg) and methyl 2-bromoacetate (394 mg, 1.3 equiv). The reaction mixture was stirred for 2 hours. The crude was purified by flash chromatography (CyHex 100% to CyHex/EtOAc 80:20) to obtain Compound 456 (844 mg) as a yellow oil.

M/Z (M[³⁵Cl]₂+H)⁺: 353.0.

### Compound 457: 1-(6-chloro-3-(4-chloro-3-fluorophenyl)-1H-pyrrolo[2,3-b]pyridin-1-yl)-2-methylpropan-2-ol

To a solution of methylmagnesium bromide 1.4 M in THF (1.74 mL, 2.5 equiv.) in THF (10 mL) at 0° C., was added a solution of Compound 456 (345 mg, 1.0 equiv.) in THF (10 mL). The reaction was stirred at 0° C. for 3 hour before a second addition of methylmagnesium bromide 1.4 M in THF (1.74 mL, 2.5 equiv.). The reaction mixture was stirred at 25° C. for 16 hour. The reaction mixture was quenched with NH₄Cl sat. aq. (30 mL) then extracted with EtOAc (2×40 mL). The organic layer was washed with brine (20 mL), dried over magnesium sulfate then concentrated to dryness. The crude was purified by flash chromatography (Merck 60®, CyHex 100% to CyHex/EtOAc 60:40) to obtain Compound 457 (173 mg) as a colorless oil.

M/Z (M[³⁵Cl]₂+H)⁺: 353.2.

### Compound 458: methyl 3-(4-chloro-3-fluorophenyl)-1-(2-hydroxy-2-methylpropyl)-1H-pyrrolo[2,3-b]pyridine-6-carboxylate

Compound 458 was prepared according to general procedure (XXIX) starting from Compound 457 (110 mg). The crude was purified by flash chromatography (CyHex 100% to CyHex/EtOAc 50:50) to obtain Compound 458 (59 mg, 50% over 3 steps) as a yellow oil.

M/Z (M[³⁵Cl]+H)⁺: 377.1.

### Compound 459: 3-(4-chloro-3-fluorophenyl)-1-(2-hydroxy-2-methylpropyl)-1H-pyrrolo[2,3-b]pyridine-6-carboxylic acid

Compound 459 was obtained (57 mg) as a yellow solid according to general procedure (IXa) starting from Compound 458 (59 mg).

M/Z (M[³⁵Cl]+H)⁺: 363.2.

### Compound 460: methyl 6-(4-(3-(4-chloro-3-fluorophenyl)-1-(2-hydroxy-2-methylpropyl)-1H-pyrrolo[2,3-b]pyridine-6-carbonyl)-3,3-dimethylpiperazin-1-yl)-2,4-dimethylnicotinate

Compound 460 was prepared according to general procedure (Xc) starting from Compound 459 (57 mg), Compound 95 (54 mg) and N,N-diisopropylethylamine (4.0 equiv). The crude residue was purified by flash chromatography (CyHex 100% to CyHex/EtOAc 50:50) to obtain Compound 460 (78 mg, 80% over 2 steps) as a yellow oil.

M/Z (M[³⁵Cl]+H)⁺: 622.5

### Example 180: 6-(4-(3-(4-chloro-3-fluorophenyl)-1-(2-hydroxy-2-methylpropyl)-1H-pyrrolo[2,3-b]pyridine-6-carbonyl)-3,3-dimethylpiperazin-1-yl)-2,4-dimethylnicotinic acid

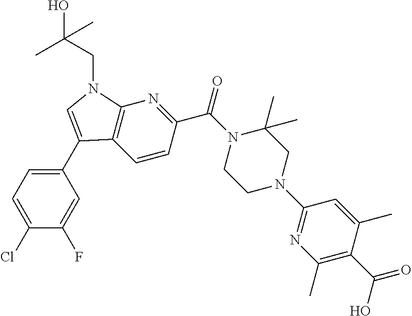 [SMILES: Cc1cc(N2CCN(C(=O)c3ccc4c(-c5ccc(Cl)c(F)c5)cn(CC(C)(C)O)c4n3)C(C)(C)C2)nc(C)c1C(=O)O]

Example 180 was prepared according to general procedure (XIb) starting from Compound 460 (78 mg). To the crude, ACN (2 mL) was added. The product was triturated and centrifuged, the supernatent was removed. Et₂O (2 mL) was added and the product was triturated and centrifuged, the supernatent was removed (action repeated twice). The product was dissolved in DCM (10 mL) then concentrated to dryness. The product was freeze-dried in EtOH/water 1:5 (75 mL) to obtain Example 180 (40 mg, 52%) as a white solid.

¹H-NMR (DMSO-d₆) δ: 1.13 (s, 6H, 2 CH₃); 1.54 (s, 6H, 2 CH₃ of piperazine); 2.27 (s, 3H, CH₃—Ar); 2.38 (s, 3H, CH₃—Ar); 3.54-3.60 (m, 2H, CH₂); 3.78-3.85 (m, 2H, CH₂); 3.92 (s, 2H, CH₂); 4.28 (s, 2H, CH₂—Ar); 4.87 (s, 1H, OH); 6.34 (s, 1H, Ar); 7.42 (d, J 8.2 Hz, 1H, Ar); 7.60-7.68 (m, 2H, Ar); 7.75-7.79 (m, 1H, Ar); 8.16 (s, 1H, Ar); 8.46 (d, J 8.2 Hz, 1H, Ar). 1 proton not observed.

M/Z (M[³⁵Cl]+H)⁺: 608.4.

### Compound 461: 6-chloro-3-(4-chloro-3-fluorophenyl)-1-((3-methyloxetan-3-yl)methyl)-1H-pyrrolo[2,3-b]pyridine

Compound 461 was prepared according to general procedure (XIII) in DMA starting from Compound 9 (175 mg) and 3-(chloromethyl)-3-methyloxetane (98 mg, 1.3 equiv). The crude was purified by flash chromatography (CyHex 100% to CyHex/EtOAc 80:20) to obtain Compound 461 (131 mg, 58%) as a white solid.

M/Z (M[³⁵Cl]₂+H)⁺: 365.1.

### Compound 462: methyl 6-(4-(3-(4-chloro-3-fluorophenyl)-1-((3-methyloxetan-3-yl)methyl)-1H-pyrrolo[2,3-b]pyridine-6-carbonyl)-3,3-dimethylpiperazin-1-yl)-2,4-dimethylnicotinate

Compound 462 was prepared according to general procedure (XXX) starting from Compound 461 (131 mg) and Compound 95 (175 mg, 2.0 equiv). The crude was purified by flash chromatography (CyHex 100% to CyHex/EtOAc 50:50) to obtain Compound 462 (58 mg, 25%) as a white solid.

### Example 181: 6-(4-(3-(4-chloro-3-fluorophenyl)-1-((3-methyloxetan-3-yl)methyl)-1H-pyrrolo[2,3-b]pyridine-6-carbonyl)-3,3-dimethylpiperazin-1-yl)-2,4-dimethylnicotinic acid

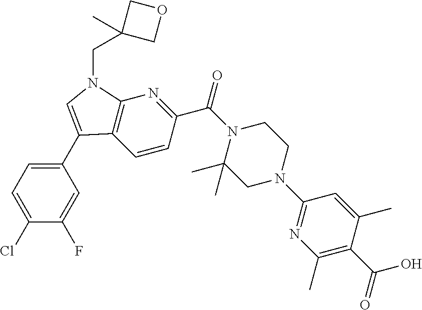 [SMILES: Cc1cc(N2CCN(C(=O)c3ccc4c(-c5ccc(Cl)c(F)c5)cn(CC5(C)COC5)c4n3)C(C)(C)C2)nc(C)c1C(=O)O]

Example 181 was prepared according to general procedure (XIb) starting from Compound 462 (29 mg). The crude was purified by preparative HPLC (30% to 70% ACN/H₂O), then further purified by flash chromatography (DCM 100% to DCM/MeOH 80:20) and triturated with diethyl ether to obtain Example 181 (6.3 mg, 22%) as a white solid.

¹H-NMR (DMSO-d₆, 300 MHz) δ: 1.25 (s, 3H, CH₃ of Oxetane); 1.53 (s, 6H, 2 CH₃); 2.24 (s, 3H, CH₃—Ar); 2.36 (s, 3H, CH₃—Ar); 3.50-3.52 (m, 2H, CH₂); 3.75-3.78 (m, 2H, CH₂); 3.91 (bs, 2H, CH₂); 4.28 (d, J 6.0 Hz, 2H, CH₂ of Oxetane); 4.56 (bs, 2H, CH₂—Ar); 4.70 (d, J 6.0 Hz, 2H, CH₂ of Oxetane); 6.30 (s, 1H, Ar); 7.42 (d, J 8.2 Hz, 1H, Ar); 7.64-7.66 (m, 2H, Ar); 7.78-7.81 (m, 1H, Ar); 8.28 (s, 1H, Ar); 8.48 (d, J 8.2 Hz, 1H, Ar). 1 proton not observed.

M/Z (M[³⁵Cl]+H)⁺: 620.4.

### Compound 463: (R)-(tetrahydrofuran-3-yl)methyl methanesulfonate

Compound 463 was obtained (125 mg, n.d.) as an orange oil according to general procedure (XXIII) from (S)-(tetrahydrofuran-3-yl)methanol (100 mg) and methanesulfonyl chloride (135 μL, 1.2 equiv).

¹H-NMR (CDCl₃, 300 MHz) δ: 1.53-1.64 (m, 1H, CHₐH_{b}—CH₂—O); 1.92-2.03 (m, 1H, CHₐH_{b}—CH₂—O); 2.56-2.63 (m, 1H, CH—CH₂—O); 3.19 (s, 3H, CH₃); 3.44-3.49 (m, 1H, CH—CHₐH_{b}—O); 3.59-3.66 (m, 1H, CH—CHₐH_{b}—O); 3.72-3.77 (m, 2H, CH₂—O); 4.08-4.19 (m, 2H, CH₂—O—SO₂).

### Compound 464: (S)-6-chloro-3-(4-chloro-3-fluorophenyl)-1-((tetrahydrofuran-3-yl)methyl)-1H-pyrrolo[2,3-b]pyridine

Compound 464 was prepared according to general procedure (XIII) in DMA starting from Compound 9 (175 mg) and Compound 463 (125 mg). The reaction was stirred at 50° C. for 17 hours. The crude was purified by flash chromatography (CyHex 100% to CyHex/EtOAc 70:30) to obtain Compound 464 (100 mg, 67%) as a white solid.

M/Z (M[³⁵Cl]₂+H)⁺: 365.1.

### Compound 465: methyl (S)-6-(4-(3-(4-chloro-3-fluorophenyl)-1-((tetrahydrofuran-3-yl)methyl)-1H-pyrrolo[2,3-b]pyridine-6-carbonyl)-3,3-dimethylpiperazin-1-yl)-2,4-dimethylnicotinate

Compound 465 was prepared according to general procedure (XXX) starting from Compound 464 (75 mg) and Compound 95 (130 mg, 2.0 equiv). The crude was purified by flash chromatography (CyHex 100% to CyHex/EtOAc 50:50) to obtain Compound 465 (73 mg, 56%) as a white solid.

M/Z (M[³⁵Cl]+H)⁺: 634.5.

### Example 182: (S)-6-(4-(3-(4-chloro-3-fluorophenyl)-1-((tetrahydrofuran-3-yl)methyl)-1H-pyrrolo[2,3-b]pyridine-6-carbonyl)-3,3-dimethylpiperazin-1-yl)-2,4-dimethylnicotinic acid

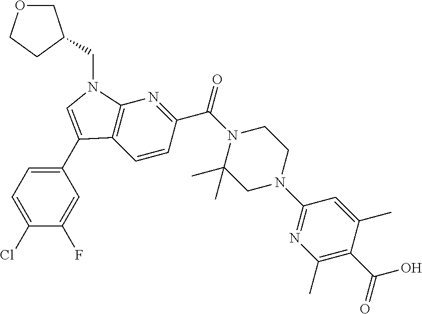 [SMILES: Cc1cc(N2CCN(C(=O)c3ccc4c(-c5ccc(Cl)c(F)c5)cn(C[C@@H]5CCOC5)c4n3)C(C)(C)C2)nc(C)c1C(=O)O]

Example 182 was prepared according to general procedure (XIb) starting from Compound 465 (73 mg). The crude was purified by preparative HPLC (30% to 70% ACN/H₂O), then freeze-dried with water to obtain Example 182 (33 mg, 46%) as a white solid.

¹H-NMR (DMSO-d₆, 300 MHz) δ: 1.54 (s, 6H, 2 CH₃); 1.64-1.74 (m, 1H, CH₂ of THF); 1.90-2.02 (m, 1H, CH₂ of THF); 2.28 (s, 3H, CH₃—Ar); 2.39 (s, 3H, CH₃—Ar); 2.85-2.94 (m, 1H, CH of THF); 3.55-3.63 (m, 3H, CH₂ of THF+CH₂); 3.66-3.71 (m, 2H, CH₂ of THF); 3.80-3.98 (m, 5H, CH₂ of THF+2 CH₂); 4.30-4.34 (m, 2H, CH₂—Ar); 6.37 (s, 1H, Ar); 7.43 (d, J 8.2 Hz, 1H, Ar); 7.64-7.65 (m, 2H, Ar); 7.76-7.80 (m, 1H, Ar); 8.34 (s, 1H, Ar); 8.48 (d, J 8.2 Hz, 1H, Ar); 12.69 (bs, 1H, COOH).

M/Z (M[³⁵Cl]+H)⁺: 620.4.

### Compound 466: (4-methyltetrahydro-2H-pyran-4-yl)methyl trifluoromethanesulfonate

Compound 466 was obtained (426 mg, n.d.) as an orange oil according to general procedure (XXIII) from (4-methyltetrahydro-2H-pyran-4-yl)methanol (200 mg) and trifluoromethanesulfonyl anhydride (477 mg, 1.1 equiv).

¹H-NMR (CDCl₃, 300 MHz) δ: 1.16 (s, 3H, CH₃); 1.35-1.42 (m, 2H, 2 CHₐH_{b}—CH₂—O); 1.57-1.67 (m, 2H, 2 CHₐH_{b}—CH₂—O); 3.60-3.68 (m, 2H, 2 CHₐH_{b}—O); 3.73-3.80 (m, 2H, 2 CHₐH_{b}—O); 4.26 (s, 2H, CH₂—OSO₂).

### Compound 467: 6-chloro-3-(4-chloro-3-fluorophenyl)-1-((4-methyltetrahydro-2H-pyran-4-yl)methyl)-1H-pyrrolo[2,3-b]pyridine

Compound 467 was prepared according to general procedure (XIII) in NMP starting from Compound 9 (175 mg) and Compound 466 (125 mg). The crude was purified by flash chromatography (CyHex 100% to CyHex/EtOAc 80:20) to obtain Compound 466 (286 mg) as a yellow oil.

M/Z (M[³⁵Cl]₂+H)⁺: 393.1.

### Compound 468: methyl 6-(4-(3-(4-chloro-3-fluorophenyl)-1-((4-methyltetrahydro-2H-pyran-4-yl)methyl)-1H-pyrrolo[2,3-b]pyridine-6-carbonyl)-3,3-dimethylpiperazin-1-yl)-2,4-dimethylnicotinate

Compound 468 was prepared according to general procedure (XXX) starting from Compound 467 (100 mg) and Compound 95 (130 mg, 2.0 equiv). The crude was purified by flash chromatography (CyHex 100% to CyHex/EtOAc 70:30) to obtain Compound 468 (166 mg, 65% over 3 steps) as an orange solid.

(M[³⁵Cl]₂+H)⁺: 662.5.

### Example 183: 6-(4-(3-(4-chloro-3-fluorophenyl)-1-((4-methyltetrahydro-2H-pyran-4-yl)methyl)-1H-pyrrolo[2,3-b]pyridine-6-carbonyl)-3,3-dimethylpiperazin-1-yl)-2,4-dimethyinicotinic acid

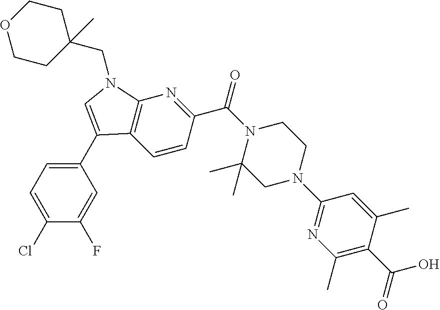 [SMILES: Cc1cc(N2CCN(C(=O)c3ccc4c(-c5ccc(Cl)c(F)c5)cn(CC5(C)CCOCC5)c4n3)C(C)(C)C2)nc(C)c1C(=O)O]

Example 183 was prepared according to general procedure (XIb) starting from Compound 468 (166 mg). The crude was purified by preparative HPLC (30% to 70% ACN/H₂O), then freeze-dried with water to obtain Example 183 (70 mg, 43%) as a white solid.

¹H-NMR (DMSO-d₆) δ: 1.03 (s, 3H, THP-CH₃); 1.26-1.34 (m, 2H, CH₂ of THP); 1.54 (s, 6H, 2 CH₃); 1.57-1.66 (m, 2H, CH₂ of THP); 2.26 (s, 3H, CH₃—Ar); 2.38 (s, 3H, CH₃—Ar); 3.48-3.59 (m, 4H, 2 CH₂ of THP); 3.72-3.81 (m, 4H, 2 CH₂); 3.92 (s, 2H, CH₂); 4.27 (s, 2H, CH₂); 6.34 (s, 1H, Ar); 7.41 (d, J 8.2 Hz, 1H, Ar); 7.61-7.67 (m, 2H, Ar); 7.78-7.82 (m, 1H, Ar); 8.21 (s, 1H, Ar); 8.47 (d, J 8.2 Hz, 1H, Ar).

M/Z (M[³⁵Cl]+H)⁺: 648.5.

### Compound 469: (S)-(tetrahydrofuran-3-yl)methyl methanesulfonate

Compound 469 was obtained (147 mg, n.d.) as an orange oil according to general procedure (XXIII) from (R)-(tetrahydrofuran-3-yl)methanol (100 mg) and methanesulfonyl chloride (135 μL, 1.2 equiv).

¹H-NMR (CDCl₃, 300 MHz) δ: 1.53-1.64 (m, 1H, CHₐH_{b}—CH₂—O); 1.92-2.03 (m, 1H, CHₐH_{b}—CH₂—O); 2.56-2.63 (m, 1H, CH—CH₂—O); 3.19 (s, 3H, CH₃); 3.44-3.49 (m, 1H, CH—CHₐH_{b}—O); 3.59-3.66 (m, 1H, CH—CHₐH_{b}—O); 3.71-3.75 (m, 2H, CH₂—O); 4.08-4.19 (m, 2H, CH₂—O—SO₂).

### Compound 470: (R)-6-chloro-3-(4-chloro-3-fluorophenyl)-1-((tetrahydrofuran-3-yl)methyl)-1H-pyrrolo[2,3-b]pyridine

Compound 469 was prepared according to general procedure (XIII) in NMP starting from Compound 9 (175 mg) and Compound 469 (147 mg). The reaction was stirred at 50° C. for 17 hours. The crude was purified by flash chromatography (CyHex 100% to CyHex/EtOAc 70:30) to obtain Compound 470 (125 mg, 74%) as a colorless oil.

M/Z (M[³⁵Cl]₂+H)⁺: 365.1.

### Compound 471: methyl (R)-6-(4-(3-(4-chloro-3-fluorophenyl)-1-((tetrahydrofuran-3-yl)methyl)-1H-pyrrolo[2,3-b]pyridine-6-carbonyl)-3,3-dimethylpiperazin-1-yl)-2,4-dimethylnicotinate

Compound 471 was prepared according to general procedure (XXX) starting from Compound 470 (125 mg) and Compound 95 (130 mg, 2.0 equiv). The crude was purified by flash chromatography (CyHex 100% to CyHex/EtOAc 50:50) to obtain Compound 471 (98 mg, 45%) as a yellow solid.

M/Z (M[³⁵Cl]+H)⁺: 634.5.

### Example 184: (R)-6-(4-(3-(4-chloro-3-fluorophenyl)-1-((tetrahydrofuran-3-yl)methyl)-1H-pyrrolo[2,3-b]pyridine-6-carbonyl)-3,3-dimethylpiperazin-1-yl)-2,4-dimethyinicotinic acid

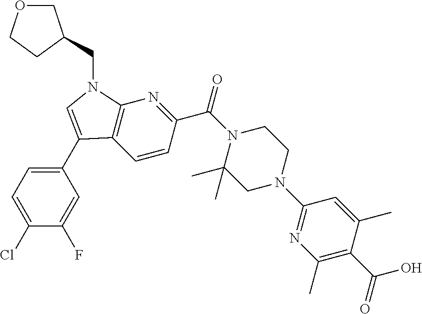 [SMILES: Cc1cc(N2CCN(C(=O)c3ccc4c(-c5ccc(Cl)c(F)c5)cn(C[C@H]5CCOC5)c4n3)C(C)(C)C2)nc(C)c1C(=O)O]

Example 184 was prepared according to general procedure (XIb) starting from Compound 471 (98 mg). The crude was purified by preparative HPLC (30% to 70% ACN/H₂O), then freeze-dried with water to obtain Example 184 (50 mg, 52%) as a white solid.

¹H-NMR (DMSO-d₆, 300 MHz) δ: 1.54 (s, 6H, 2 CH₃); 1.64-1.74 (m, 1H, CH₂ of THF); 1.90-2.02 (m, 1H, CH₂ of THF); 2.27 (s, 3H, CH₃—Ar); 2.38 (s, 3H, CH₃—Ar); 2.85-2.94 (m, 1H, CH of THF); 3.55-3.63 (m, 3H, CH₂ of THF+CH₂); 3.66-3.71 (m, 2H, CH₂ of THF); 3.80-3.98 (m, 5H, CH₂ of THF+2 CH₂); 4.25-4.37 (m, 2H, CH₂—Ar); 6.35 (s, 1H, Ar); 7.43 (d, J 8.2 Hz, 1H, Ar); 7.64-7.65 (m, 2H, Ar); 7.76-7.80 (m, 1H, Ar); 8.35 (s, 1H, Ar); 8.47 (d, J 8.2 Hz, 1H, Ar); 12.68 (bs, 1H, COOH).

M/Z (M[³⁵Cl]+H)⁺: 620.4.

### Compound 472: 3-(tert-butyl)-5-methoxy-1H-pyrrolo[3,2-b]pyridine

To a solution of 5-hydrazineyl-2-methoxypyridine hydrochloride (1.00 g, 1.0 equiv) in Water (25 mL) was added 3,3-dimethylbutanal (627 mg, 1.10 equiv) and H₂SO₄ (12 μL, 0.04 equiv). The reaction was heated at 100° C. for 6 hours. The mixture was carefully poured in a saturated aqueous solution of NaHCO₃ (150 mL) and extracted thrice with EtAOc (50 mL), dried over magnesium sulfate then concentrated to dryness.

The crude was purified by flash chromatography (Merck 60®, CyHex 100% to CyHex/CyHex/EtOAc 50/50) to obtain Compound 472 (509 mg, 44%) as a yellow gum.

M/Z (M+H)⁺: 205.1.

### Compound 473: 3-(tert-butyl)-1-(4-chloro-3-fluorophenyl)-5-methoxy-1H-pyrrolo[3,2-b]pyridine

In a sealed tube, Compound 472 (500 mg, 1.0 equiv) in toluene (15 mL) were added 4-bromo-1-chloro-2-fluorobenzene (672 μL 2.2 equiv), (1R,2R)—N1,N2-dimethylcyclohexane-1,2-diamine (154 μL, 0.4 equiv) and potassium phosphate (2.18 g, 4.2 equivl). The reaction mixture was sparged by Ar for 10 min before addition of copper(I) iodide (46 mg, 0.1 equiv). The reaction mixture was heated at 120° C. for 18 hours. After addition of water (100 mL) reaction mixture was extracted thrice with EtOAc (200 mL). The organic layer was dried over magnesium sulfate and concentrated to dryness. The crude was purified by flash chromatography (Merck 60®, CyHex 100% to CyHex/EtOAc 90:10) to Compound 473 (790 mg) yellow oil.

M/Z (M[³⁵Cl]+H)⁺: 333.2.

### Compound 474: 3-(tert-butyl)-1-(4-chloro-3-fluorophenyl)-1H-pyrrolo[3,2-b]pyridin-5-ol

To a solution of Compound 473 (790 mg, 1.0 equiv) in DCM (15 mL) at 0° C. was added BBr₃ (1M in DCM) (14.2 mL, 6.0 equiv). The reaction mixture was stirred at 25° C. for 18 hours. The reaction mixture was carefully poured onto NaHCO₃ sat. aq. (200 mL) and extracted thrice with EtOAc (200 mL). The organic layer was dried over magnesium sulfate and concentrated to dryness. The crude was purified by flash chromatography (Merck 60®, CyHex100% to CyHex/EtOAc 0:100) to obtain Compound 474 (470 mg, 60% over 2 steps) as a white solid.

M/Z (M[³⁵Cl]+H)⁺: 319.2.

### Compound 475: 3-(tert-butyl)-1-(4-chloro-3-fluorophenyl)-1H-pyrrolo[3,2-b]pyridin-5-yl trifluoromethanesulfonate

To a suspension of Compound 474 (704 mg, 1.0 equiv) in DCM (60 mL) at 0° C. was added pyridine (5.0 mL) and trifluoromethanesulfonic anhydride (821 μL, 2.2 equiv). The reaction mixture was stirred at 25° C. for 4 hours. The reaction mixture was hydrolysed with NaHCO₃ sat. Aq. (200 mL) and extracted thrice with EtOAc (200 mL). The crude was purified by flash chromatography (Merck 60®, CyHex 100% to CyHex/EtOAc 50:50) to obtain Compound 475 (930 mg, 93%) as a white solid. M/Z (M[³⁵Cl]+H)⁺: 451.2.

### Compound 476: methyl 6-(4-(3-(tert-butyl)-1-(4-chloro-3-fluorophenyl)-1H-pyrrolo[3,2-b]pyridine-5-carbonyl)-3,3-dimethylpiperazin-1-yl)-2,4-dimethylnicotinate

Compound 476 was prepared according to general procedure (XXX) starting from Compound 475 (150 mg) and Compound 95 (209 mg, 2.0 equiv). The crude was purified by flash chromatography (CyHex 100% to CyHex/EtOAc 80:20) to obtain Compound 476 (130 mg, 64%) as a yellow oil.

M/Z (M[³⁵Cl]+H)⁺: 606.5

### Example 185: 6-(4-(3-(tert-butyl)-1-(4-chloro-3-fluorophenyl)-1H-pyrrolo[3,2-b]pyridine-5-carbonyl)-3,3-dimethylpiperazin-1-yl)-2,4-dimethylnicotinic acid

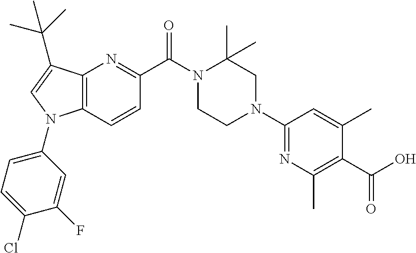 [SMILES: Cc1cc(N2CCN(C(=O)c3ccc4c(n3)c(C(C)(C)C)cn4-c3ccc(Cl)c(F)c3)C(C)(C)C2)nc(C)c1C(=O)O]

Example 185 was prepared according to general procedure (XIc) starting from Compound 476 (130 mg). The crude was purified by preparative HPLC (30% to 70% ACN/H₂O), then freeze-dried with water to obtain Example 185 (15 mg, 12%) as a white solid.

¹H-NMR (DMSO-d₆, 300 MHz) δ: 1.53 (s, 9H, tBu); 1.54 (s, 6H, 2 CH₃ of piperazine); 2.27 (s, 3H, CH₃—Ar); 2.38 (s, 3H, CH₃—Ar); 3.55-3.61 (m, 2H, CH₂); 3.87-3.94 (m, 2H, 2 CH₂); 6.31 (s, 1H, Ar); 7.46 (d, J 8.6 Hz, 1H, Ar); 7.55-7.59 (m, 1H, Ar); 7.76-7.87 (m, 3H, Ar); 8.10 (d, J 8.6 Hz, 1H, Ar).

M/Z (M[³⁵Cl]+H)⁺: 592.3.

### Compound 477: methyl 6-(4-(3-(4-chloro-3-fluorophenyl)-1-isobutyl-5-methyl-1H-pyrrolo[2,3-b]pyridine-6-carbonyl)piperazin-1-yl)-2,4-dimethylnicotinate

Compound 477 was prepared according to general procedure (Xa) starting from Compound 437 (60 mg) and Compound 243 (52 mg, 1.1 equiv), and using N,N-diisopropylethylamine (4.0 equiv). The crude was purified by flash chromatography (Merck 60®, CyHex 100% to CyHex/EtOAc: 60:40) to obtain Compound 477 (90 mg) as a white solid.

M/Z (M[³⁵Cl]+H)⁺: 592.5

### Example 186: 6-(4-(3-(4-chloro-3-fluorophenyl)-1-isobutyl-5-methyl-1H-pyrrolo[2,3-b]pyridine-6-carbonyl)piperazin-1-yl)-2,4-dimethylnicotinic acid

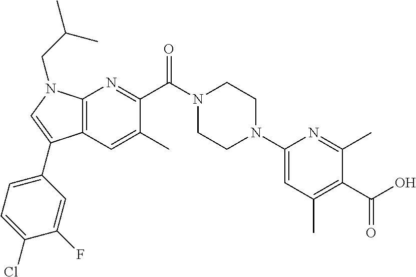 [SMILES: Cc1cc2c(-c3ccc(Cl)c(F)c3)cn(CC(C)C)c2nc1C(=O)N1CCN(c2cc(C)c(C(=O)O)c(C)n2)CC1]

Example 186 was prepared according to general procedure (XIb) starting from Compound 477 (90 mg). The crude was purified by preparative HPLC (40% to 80% ACN/H₂O), then freeze-dried with water to obtain Example 186 (45 mg, 51% over 2 steps) as a white solid.

¹H-NMR (DMSO-d₆, 300 MHz) δ: 0.85 (d, J 6.6 Hz, 6H, (CH₃)₂); 2.17-2.23 (m, 1H, CH); 2.24 (s, 3H, CH₃); 2.34 (s, 3H, CH₃); 2.38 (s, 3H, CH₃); 3.20-3.23 (m, 2H, CH₂); 3.48-3.52 (m, 2H, CH₂); 3.64-3.70 (m, 2H, CH₂); 3.76-3.81 (m, 2H, CH₂); 4.07 (d, J 6.6 Hz, 2H, CH₂); 6.55 (s, 1H, Ar); 7.58-7.63 (m, 2H, Ar); 7.75-7.77 (m, 1H, Ar); 8.18 (s, 1H, Ar); 8.31 (s, 1H, Ar); 12.77 (bs, 1H, CO₂H).

M/Z (M[³⁵Cl]+H)⁺: 578.4.

### Compound 478: tert-butyl 4-(((methylsulfonyl)oxy)methyl)piperidine-1-carboxylate

Compound 478 was obtained (748 mg), as a colorless oil according to general procedure (XXIII) starting from tert-butyl 4-(hydroxymethyl)piperidine-1-carboxylate (500 mg) and methanesulfonyl chloride (235 μL, 1.3 equiv).

M/Z (M-tBu+H)⁺: 238.1.

### Compound 479: tert-butyl 4-((4-(ethoxycarbonyl)-1H-pyrazol-1-yl)methyl)piperidine-1-carboxylate

To a solution of Compound 478 (640 mg, 1.0 equiv) in ACN (30 mL) was added potassium carbonate (422 mg, 1.4 equiv) and ethyl 4-pyrazolecarboxylate (428 mg, 1.4 equiv). The reaction was heated at 80° C. for 48 hours. The reaction mixture was quenched with water (40 mL) then extracted with EtOAc (2×40 mL). The organic layer was washed with brine (10 mL), dried over magnesium sulfate then concentrated to dryness. The crude was purified by flash chromatography Merck 60®, CyHex 100% to EtOAc 100%) to obtain Compound 479 (250 mg) as a colorless oil.

M/Z (M-tBu+H)⁺: 282.2.

### Compound 480: ethyl 1-(piperidin-4-ylmethyl)-1H-pyrazole-4-carboxylate

Compound 480 was prepared according to general procedure (XVIIIc) starting from Compound 479 (250 mg). After concentration to dryness, the obtained residue, was suspended in EtOAc (40 mL) then extracted with aqueous HCl 1N (30 mL). The aqueous acid solution was washed with EtOAc (20 mL) then was alkalinized with K₂CO₃ sat. aq. solution until pH=9. The aqueous layer was extracted with EtOAc (2×30 mL). The organic layer was washed with brine (20 mL), dried over magnesium sulfate then concentrated to dryness to obtain Compound 480 (65 mg, 12% over 3 steps) as a colorless oil.

M/Z (M+H)⁺: 238.2.

### Compound 481: ethyl 1-((1-(1-(tert-butyl)-3-(4-chloro-3-fluorophenyl)-1H-pyrrolo[2,3-b]pyridine-6-carbonyl)piperidin-4-yl)methyl)-1H-pyrazole-4-carboxylate

Compound 481 was prepared according to general procedure (Xa) starting from Compound 480 (60 mg, 1.0 equiv) and Compound 78 (87 mg, 1.0 equiv), and using N,N-diisopropylethylamine (4.0 equiv). The crude was purified by flash chromatography (Merck 60®, CyHex 100% to CyHex/EtOAc: 70:30) to obtain Compound 481 (131 mg, 91%) as a white solid.

M/Z (M[³⁵Cl]+H)⁺: 566.4.

### Example 187: 1-((1-(1-(tert-butyl)-3-(4-chloro-3-fluorophenyl)-1H-pyrrolo[2,3-b]pyridine-6-carbonyl)piperidin-4-yl)methyl)-1H-pyrazole-4-carboxylic acid

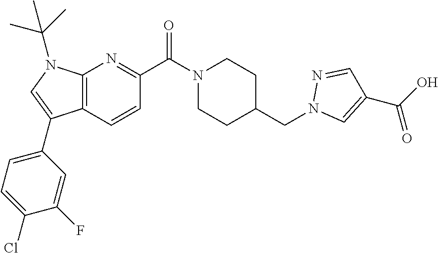 [SMILES: CC(C)(C)n1cc(-c2ccc(Cl)c(F)c2)c2ccc(C(=O)N3CCC(Cn4cc(C(=O)O)cn4)CC3)nc21]

Example 187 was prepared according to general procedure (IXa) starting from Compound 481 (130 mg). The crude was purified by preparative HPLC (40% to 80% ACN/H₂O), then freeze-dried with water to obtain Example 187 (65 mg, 53%) as a white solid.

¹H-NMR (DMSO-d₆, 300 MHz) δ: 1.20-1.29 (m, 2H, CH₂); 1.43-1.47 (m, 1H, CH); 1.54-1.63 (m, 1H, CH); 1.77 (s, 9H, (CH₃)₃); 2.12-2.19 (m, 1H, CH); 2.76-2.85 (m, 1H, CH); 3.01-3.10 (m, 1H, CH); 3.97-4.02 (m, 1H, CH); 4.11 (d, J 6.6 Hz, 2H, CH₂); 4.49-4.54 (m, 1H, CH); 7.38 (d, J 8.2 Hz, 1H, Ar); 7.58-7.66 (m, 2H, Ar); 7.78-7.83 (m, 2H, Ar); 8.19 (s, 1H, pyr), 8.23 (s, 1H, pyr); 8.41 (d, J 8.2 Hz, 1H, Ar); 12.30 (bs, 1H, CO₂H).

M/Z (M[³⁵Cl]+H)⁺:538.4.

### Compound 482: 4-methyl-1H-pyrrolo[2,3-b]pyridine 7-oxide hydrochloride

Compound 482 was obtained (1.42 g) as a brown solid according to general procedure (XIXa) from 4-methyl-1H-pyrrolo[2,3-b]pyridine (1.0 g) and 3-chlorobenzoperoxoic acid<77% (1.9 g, 1.0 equiv).

M/Z (M+H)⁺: 149.1.

### Compound 483: 4-methyl-1H-pyrrolo[2,3-b]pyridine-6-carbonitrile

Compound 483 was prepared according to general procedure (XX) starting from Compound 482 (1.42 g). The crude residue was purified by flash chromatography (CyHex 100% to CyHex/EtOAc 70:30) to obtain Compound 483 (660 mg) as a beige solid.

M/Z (M+H)⁺: 158.0.

### Compound 484: 3-bromo-4-methyl-1H-pyrrolo[2,3-b]pyridine-6-carbonitrile

To a solution of Compound 483 (640 mg, 1.0 equiv) in DMF (13 mL) was added N-bromosuccinimide (725 mg, 1.0 equiv). The reaction was stirred at 25° C. for 4 hours. The reaction mixture was hydrolyzed with water (100 mL) then extracted with EtOAc (2×50 mL). The organic layer was washed with 10% aq. Na₂S₂O₃ (50 mL), water (15 mL), dried over magnesium sulfate then concentrated to dryness. The crude was purified by flash chromatography (Merck 60®, CyHex 100% to EtOAc 100%+DCM 3% as additive) to obtain Compound 484 (760 mg, 42% over 3 steps) as an orange solid.

M/Z (M[⁸¹Br]+H)⁺: 237.9.

### Compound 485: 3-bromo-1-isobutyl-4-methyl-1H-pyrrolo[2,3-b]pyridine-6-carbonitrile

Compound 485 was prepared according to general procedure (XIIIb) from Compound 484 (755 mg) and 1-iodo-2-methylpropane (740 μL, 2.0 equiv). The reaction mixture was heated at 50° C. for 17 hours. The crude was purified by flash chromatography (Merck 60®, CyHex 100% to CyHex/EtOAc 90:10) to obtain Compound 485 (514 mg, 55%) as a white solid.

M/Z (M[⁷⁹Br]+H)⁺: 292.2.

### Compound 486: 3-(4-chloro-3-fluorophenyl)-1-isobutyl-4-methyl-1H-pyrrolo[2,3-b]pyridine-6-carbonitrile

Compound 486 was prepared according to general procedure (XVIIa) starting from Compound 485 (410 mg) and (4-chloro-3-fluorophenyl)boronic acid (294 mg, 1.2 equiv). The crude was purified by flash chromatography (Merck 60®, CyHex 100% to CyHex/EtOAc 80:20) to obtain Compound 486 (370 mg, 77%) as a white solid.

M/Z (M[³⁵Cl]+H)⁺: 342.2.

### Compound 487: 3-(4-chloro-3-fluorophenyl)-1-isobutyl-4-methyl-1H-pyrrolo[2,3-b]pyridine-6-carboxamide

Compound 487 was obtained (355 mg, 91%), as a white solid according to general procedure (VII) starting from Compound 486 (370 mg).

M/Z (M[³⁵Cl]+H)⁺: 360.2.

### Compound 488: methyl 3-(4-chloro-3-fluorophenyl)-1-isobutyl-4-methyl-1H-pyrrolo[2,3-b]pyridine-6-carboxylate

Compound 488 was prepared according to general procedure (VIII) starting from Compound 487 (350 mg). The residue was solubilized in EtOAc (100 mL), washed with water (100 mL), dried over magnesium sulfate and concentrated. The crude was purified by flash chromatography (Merck 60®, CyHex 100% to CyHex/EtOAc 80:20) to obtain Compound 488 (345 mg, 95%) as a yellow solid.

M/Z (M[³⁵Cl]+H)⁺: 375.2.

### Compound 489: 3-(4-chloro-3-fluorophenyl)-1-isobutyl-4-methyl-1H-pyrrolo[2,3-b]pyridine-6-carboxylic acid

Compound 489 was obtained (310 mg, 94%) as a yellow solid according to general procedure (IXa) starting from Compound 488 (343 mg).

M/Z (M[³⁵Cl]+H)⁺: 361.2.

### Compound 490: methyl 6-(4-(3-(4-chloro-3-fluorophenyl)-1-isobutyl-4-methyl-1H-pyrrolo[2,3-b]pyridine-6-carbonyl)-3,3-dimethylpiperazin-1-yl)-2,4-dimethylnicotinate

Compound 490 was prepared according to general procedure (Xa) starting from Compound 489 (60 mg) and Compound 95 (57 mg, 1.1 equiv), and using N,N-diisopropylethylamine (4.0 equiv). The crude was purified by flash chromatography (Merck 60®, CyHex 100% to CyHex/EtOAc 60:40) to obtain Compound 490 (100 mg, 97%) as a yellow oil.

M/Z (M[³⁵Cl]+H)⁺: 620.6.

### Example 188: 6-(4-(3-(4-chloro-3-fluorophenyl)-1-isobutyl-4-methyl-1H-pyrrolo[2,3-b]pyridine-6-carbonyl)-3,3-dimethylpiperazin-1-yl)-2,4-dimethyinicotinic acid

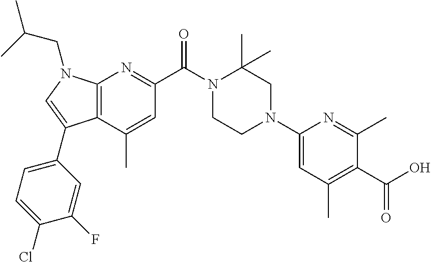 [SMILES: Cc1cc(N2CCN(C(=O)c3cc(C)c4c(-c5ccc(Cl)c(F)c5)cn(CC(C)C)c4n3)C(C)(C)C2)nc(C)c1C(=O)O]

Example 188 was prepared according to general procedure (XIb) starting from Compound 490 (100 mg). The crude was purified by flash chromatography (Interchim® 20 μm, DCM 100% to DCM/MeOH 60:40) then further purified by preparative HPLC (40% to 80% ACN/H₂O) and freeze-dried with water to obtain Example 188 (15 mg, 15%) as a white solid.

¹H-NMR (DMSO-d₆, 300 MHz) δ: 0.90 (d, J 7.2 Hz, 6H, (CH₃)₂); 1.52 (s, 6H, (CH₃)₂); 2.20-2.24 (m, 1H, CH); 2.25 (s, 3H, CH₃); 2.37 (s, 3H, CH₃); 2.38 (s, 3H, CH₃); 3.54-3.57 (m, 2H, CH₂); 3.77-3.81 (m, 2H, CH₂); 3.90 (bs, 2H, CH₂); 4.11 (d, J 7.2 Hz, 2H, CH₂); 6.32 (s, 1H, Ar); 7.14 (s, 1H, Ar); 7.33 (dd, J 2.1, 8.5 Hz, 1H, Ar); 7.54 (dd, J 2.1, 7.5 Hz, 1H, Ar); 7.62 (t, J 8.5 Hz, 1H, Ar); 7.77 (s, 1H, Ar); 12.68 (bs, 1H, COOH).

M/Z (M[³⁵Cl]+H)⁺: 606.5.

### Compound 491: methyl 6-(4-(3-(4-chloro-3-fluorophenyl)-1-isobutyl-4-methyl-1H-pyrrolo[2,3-b]pyridine-6-carbonyl)piperazin-1-yl)-2,4-dimethylnicotinate

Compound 491 was prepared according to general procedure (Xa) starting from Compound 489 (60 mg) and Compound 243 (53 mg, 1.1 equiv), and using N,N-diisopropylethylamine (4.0 equiv). The crude was purified by flash chromatography (Merck 60®, CyHex 100% to CyHex/EtOAc 60:40) to obtain Compound 491 (92 mg) as a colorless oil.

M/Z (M[³⁵Cl]+H)⁺: 592.5

### Example 189: 6-(4-(3-(4-chloro-3-fluorophenyl)-1-isobutyl-4-methyl-1H-pyrrolo[2,3-b]pyridine-6-carbonyl)piperazin-1-yl)-2,4-dimethylnicotinic acid

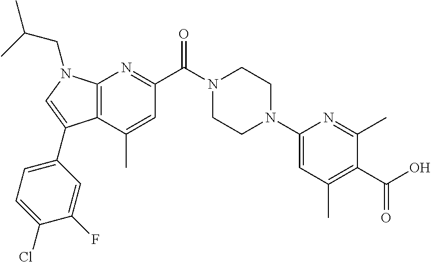 [SMILES: Cc1cc(N2CCN(C(=O)c3cc(C)c4c(-c5ccc(Cl)c(F)c5)cn(CC(C)C)c4n3)CC2)nc(C)c1C(=O)O]

Example 189 was prepared according to general procedure (XIb) starting from Compound 491 (90 mg). The crude was purified by preparative HPLC (30% to 70% ACN/H₂O) and freeze-dried with water to obtain Example 189 (50 mg, 52% over 2 steps) as a white solid.

¹H-NMR (DMSO-d₆, 300 MHz) δ: 0.89 (d, J 6.9 Hz, 6H, (CH₃)₂); 2.19-2.25 (m, 1H, CH); 2.26 (s, 3H, CH₃); 2.37 (s, 3H, CH₃); 2.39 (s, 3H, CH₃); 3.64-3.77 (m, 8H, (CH₂)₄); 4.09 (d, J 6.9 Hz, 2H, CH₂); 6.58 (s, 1H, Ar); 7.22 (s, 1H, Ar); 7.34 (dd, J 1.5, 8.2 Hz, 1H, Ar); 7.55 (dd, J 1.5, 10.5 Hz, 1H, Ar); 7.62 (t, J 8.2 Hz, 1H, Ar); 7.79 (s, 1H, Ar); 12.78 (bs, 1H, CO₂H).

M/Z (M[³⁵Cl]+H)⁺: 578.4.

### Compound 492: 5H-pyrrolo[2,3-b]pyrazine-3-carbonitrile

Compound 492 was prepared according to general procedure (V) starting from 3-chloro-5H-pyrrolo[2,3-b]pyrazine (985 mg). The reaction mixture was subjected to microwave irradiation at 150° C. for 1 hour. The crude was purified by flash chromatography (DCM 100% to EtOAc 100%) then triturated twice in CyHex (15 mL) to obtain Compound 492 (960 mg) as an orange solid.

M/Z (M+H)⁺: 144.9.

### Compound 493: 7-bromo-5H-pyrrolo[2,3-b]pyrazine-3-carbonitrile

Compound 493 was obtained (950 mg, 75%) as a brown oil according to general procedure (XIV) starting from Compound 492 (950 mg).

M/Z (M[⁸¹Br]+H)⁺: 224.9.

### Compound 494: 7-bromo-5-isobutyl-5H-pyrrolo[2,3-b]pyrazine-3-carbonitrile

Compound 494 was prepared according to general procedure (XIIIb) from Compound 493 (608 mg) and 1-iodo-2-methylpropane (630 μL, 2.0 equiv). The reaction mixture was heated at 50° C. for 17 hours. The crude was purified by flash chromatography (Merck 60®, CyHex 100% to CyHex/EtOAc 70:30) to obtain Compound 494 (321 mg, 42%) as a yellow solid.

M/Z (M[⁷⁹Br]+H)⁺: 281.2.

### Compound 495: 7-(4-chloro-3-fluorophenyl)-5-isobutyl-5H-pyrrolo[2,3-b]pyrazine-3-carbonitrile

Compound 495 was prepared according to general procedure (XVIIa) starting from Compound 494 (407 mg) and (4-chloro-3-fluorophenyl)boronic acid (305 mg, 1.2 equiv). The crude was purified by flash chromatography (CyHex 100% to CyHex/EtOAc 70:30) to obtain Compound 495 (436 mg, 91%) as a yellow solid.

M/Z (M[³⁵Cl]+H)⁺: 329.2.

### Compound 496: 7-(4-chloro-3-fluorophenyl)-5-isobutyl-5H-pyrrolo[2,3-b]pyrazine-3-carboxamide

Compound 496 was prepared according to general procedure (VII) starting from Compound 495 (425 mg). The crude was purified by flash chromatography (Merck 60®, CyHex 100% to CyHex/EtOAc 20:80) to obtain Compound 496 (110 mg, 25%) as a yellow solid.

M/Z (M[³⁵Cl]+H)⁺: 347.2

### Compound 497: methyl 7-(4-chloro-3-fluorophenyl)-5-isobutyl-5H-pyrrolo[2,3-b]pyrazine-3-carboxylate

Compound 497 was prepared according to general procedure (VIII) starting from Compound 496 (100 mg). The residue was solubilized in EtOAc (100 mL), washed with water (100 mL), dried over magnesium sulfate and concentrated. The crude was purified by flash chromatography (Merck 60®, CyHex 100% to CyHex/EtOAc 60:40) to obtain Compound 497 (70 mg, 61%) as a yellow solid.

M/Z (M[³⁵Cl]+H)⁺: 362.2.

### Compound 498: 7-(4-chloro-3-fluorophenyl)-5-isobutyl-5H-pyrrolo[2,3-b]pyrazine-3-carboxylic acid

Compound 498 was obtained (55 mg, 82%) as a yellow solid according to general procedure (IXa) starting from Compound 497 (70 mg).

M/Z (M[³⁵Cl]+H)⁺: 348.2.

### Compound 499: methyl 6-(4-(7-(4-chloro-3-fluorophenyl)-5-isobutyl-5H-pyrrolo[2,3-b]pyrazine-3-carbonyl)-3,3-dimethylpiperazin-1-yl)-2,4-dimethylnicotinate

Compound 499 was prepared according to general procedure (Xa) starting from Compound 498 (51 mg) and Compound 95 (51 mg, 1.1 equiv), and using N,N-diisopropylethylamine (4.0 equiv). The crude was purified by flash chromatography (Merck 60®, CyHex 100% to CyHex/EtOAc 70:30) to obtain Compound 499 (75 mg) as a yellow oil.

M/Z (M[³⁵Cl]+H)⁺: 607.5.

### Example 190: 6-(4-(7-(4-chloro-3-fluorophenyl)-5-isobutyl-5H-pyrrolo[2,3-b]pyrazine-3-carbonyl)-3,3-dimethylpiperazin-1-yl)-2,4-dimethylnicotinic acid

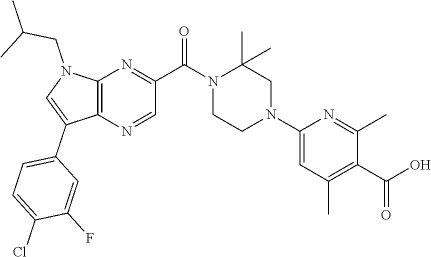 [SMILES: Cc1cc(N2CCN(C(=O)c3cnc4c(-c5ccc(Cl)c(F)c5)cn(CC(C)C)c4n3)C(C)(C)C2)nc(C)c1C(=O)O]

Example 190 was prepared according to general procedure (XIb) starting from Compound 499 (75 mg). The crude was purified by flash chromatography (Interchim® 20 μm, DCM 100% to DCM/MeOH 60:40) then further purified by preparative HPLC (30% to 70% ACN/H₂O) and freeze-dried with water to obtain Example 190 (34 mg, 39% over 2 steps) as a light-yellow solid.

¹H-NMR (DMSO-d₆, 300 MHz) δ: 0.91 (d, J 7.0 Hz, 6H, (CH₃)₂); 1.51 (s, 6H, (CH₃)₂); 2.23-2.28 (m, 4H, CH₃, CH); 2.37 (s, 3H, CH₃); 3.55-3.58 (m, 2H, CH₂); 3.81-3.83 (m, 2H, CH₂); 3.92 (bs, 2H, CH₂); 4.14 (d, J 7.0 Hz, 2H, CH₂); 6.33 (s, 1H, Ar); 7.67 (t, J 8.4 Hz, 1H, Ar); 8.13 (dd, J 1.6, 8.4 Hz, 1H, Ar); 8.27 (dd, J 1.6, 11.4 Hz, 1H, Ar); 8.75 (s, 1H, Ar); 8.81 (s, 1H, Ar); 12.76 (bs, 1H, COOH).

M/Z (M[³⁵Cl]+H)⁺: 593.5.

### Compound 500: 1-(2,6-dichloropyridin-3-yl)-1-(3,4-difluorophenyl)ethan-1-ol

Compound 500 was obtained (5.50 g, n.d.) as an orange oil according to general procedure (I) starting from 2,6-dichloropyridine (2.20 g) and 1-(3,4-difluorophenyl)ethan-1-one (2.50 g, 1.1 equiv).

Compound 500 is contaminated with 1-(2,6-dichloropyridin-4-yl)-1-(3,4-difluorophenyl)ethan-1-ol (around 20%).

M/Z (M[³⁵Cl]₂+H)⁺: 304.0

### Compound 501: 2,6-dichloro-3-(1-(3,4-difluorophenyl)vinyl)pyridine

Compound 501 was prepared according to general procedure (II) starting from Compound 500 (5.50 g). The crude was purified by flash chromatography (CyHex 100% to CyHex/EtOAc 90:10) to obtain Compound 501 (3.65 g, 88% over 2 steps) as a colorless oil. Compound 501 is contaminated with 2,6-dichloro-4-(1-(3,4-difluorophenyl)vinyl)pyridine (around 20%).

M/Z (M[³⁵Cl₂]+H)⁺: 286.0

### Compound 502: 2,6-dichloro-3-(2-(3,4-difluorophenyl)oxiran-2-yl)pyridine

Compound 502 was obtained (5.20 g, n.d) as a yellow oil according to general procedure (III) starting from Compound 501 (3.65 g). Compound 502 is contaminated with 2,6-dichloro-4-(2-(3,4-difluorophenyl)oxiran-2-yl)pyridine (around 20%).

M/Z (M[³⁵Cl]₂+H)⁺: 302.8

### Compound 503: 1-(tert-butyl)-6-chloro-3-(3,4-difluorophenyl)-1H-pyrrolo[2,3-b]pyridine

Compound 503 was prepared according to general procedure (IV) starting from Compound 502 (3.11 g) and tert-butylamine (2.70 mL, 2.5 equiv) in DMA. The crude was purified by flash chromatography (CyHex 100% to CyHex/EtOAc 98:02) to obtain Compound 503 (1.22 g, 37%) as a white solid.

M/Z (M[³⁵Cl]+H)⁺: 321.1

### Compound 504: methyl 6-(4-(1-(tert-butyl)-3-(3,4-difluorophenyl)-1H-pyrrolo[2,3-b]pyridine-6-carbonyl)-3,3-dimethylpiperazin-1-yl)-2,4-dimethylnicotinate

Compound 504 was prepared according to general procedure (XXX) starting from Compound 503 (155 mg) and Compound 95 (294 mg, 2.0 equiv). The crude was purified by flash chromatography (CyHex 100% to CyHex/EtOAc 80:20) to obtain Compound 504 (183 mg, 66%) as a white solid.

M/Z (M+H)⁺: 590.5.

### Example 191: 6-(4-(1-(tert-butyl)-3-(3,4-difluorophenyl)-1H-pyrrolo[2,3-b]pyridine-6-carbonyl)-3,3-dimethylpiperazin-1-yl)-2,4-dimethylnicotinic acid

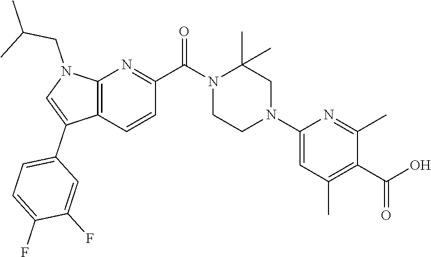 [SMILES: Cc1cc(N2CCN(C(=O)c3ccc4c(-c5ccc(F)c(F)c5)cn(CC(C)C)c4n3)C(C)(C)C2)nc(C)c1C(=O)O]

Example 191 was prepared according to general procedure (XIb) starting from Compound 504 (180 mg). The crude was purified by preparative HPLC (30% to 70% ACN/H₂O) and freeze-dried with water to obtain Example 191 (70 mg, 38%) as a white solid.

¹H-NMR (DMSO-d₆, 300 MHz) δ: 1.54 (s, 6H, 2 CH₃ of piperazine); 1.82 (s, 9H, tBu); 2.27 (s, 3H, CH₃—Ar); 2.38 (s, 3H, CH₃—Ar); 3.52-3.58 (m, 2H, CH₂); 3.83-3.88 (m, 2H, CH₂); 3.92 (s, 2H, CH₂); 6.32 (s, 1H, Ar); 7.39 (d, J 8.2 Hz, 1H, Ar); 7.45-7.54 (m, 1H, Ar); 7.58-7.63 (m, 1H, Ar); 7.78-7.86 (m, 1H, Ar); 8.13 (s, 1H, Ar); 8.40 (d, J 8.2 Hz, 1H, Ar); 12.69 (s, 1H, COOH).

M/Z (M+H)⁺: 576.3.

### Compound 505: 6-chloro-3-(4-chloro-3-fluorophenyl)-1-((6-methylpyridin-2-yl)methyl)-1H-pyrrolo[2,3-b]pyridine

Compound 505 was prepared according to general procedure (XIII) in DMF from Compound 9 (150 mg) and 2-(bromomethyl)-6-methylpyridine (149 mg, 1.5 equiv). The reaction mixture was stirred for 2 hours. The crude was purified by flash chromatography (CyHex 100% to CyHex/EtOAc 80:20) to obtain Compound 505 (157 mg, 76%) as a white solid.

M/Z (M[³⁵Cl]₂+H)⁺: 386.2.

### Compound 506: methyl 6-(4-(3-(4-chloro-3-fluorophenyl)-1-((6-methylpyridin-2-yl)methyl)-1H-pyrrolo[2,3-b]pyridine-6-carbonyl)-3,3-dimethylpiperazin-1-yl)-2,4-dimethylnicotinate

Compound 506 was prepared according to general procedure (XXX) starting from Compound 505 (157 mg) and Compound 95 (255 mg, 2.0 equiv). The crude was purified by flash chromatography (CyHex 100% to CyHex/EtOAc 60:40) to obtain Compound 506 (175 mg, 66%) as a yellow oil.

M/Z (M[³⁵Cl]+H)⁺: 655.4.

### Example 192: 6-(4-(3-(4-chloro-3-fluorophenyl)-1-((6-methylpyridin-2-yl)methyl)-1H-pyrrolo[2,3-b]pyridine-6-carbonyl)-3,3-dimethylpiperazin-1-yl)-2,4-dimethylnicotinic acid hydrochloride

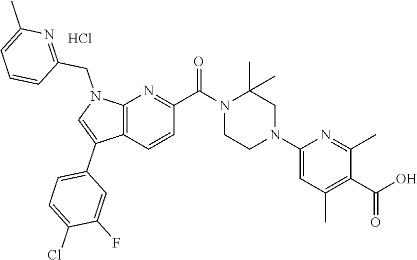 [SMILES: Cc1cccc(Cn2cc(-c3ccc(Cl)c(F)c3)c3ccc(C(=O)N4CCN(c5cc(C)c(C(=O)O)c(C)n5)CC4(C)C)nc32)n1 ; Cl]

Example 192 was prepared according to general procedure (XIb) starting from Compound 506 (175 mg). The crude was purified by preparative HPLC (20% to 60% ACN/H₂O) and freeze-dried from HCl 1M (5 mL) and water (100 mL) to obtain Example 192 (35 mg, 19%) as a beige solid.

¹H-NMR (DMSO-d₆, 300 MHz) δ: 1.52 (s, 6H, 2 CH₃ of piperazine); 2.32 (s, 3H, CH₃—Ar); 2.46 (s, 3H, CH₃—Ar); 2.57 (s, 3H, CH₃—Ar); 3.41-3.47 (m, 2H, CH₂); 3.68-3.73 (m, 4H, 2 CH₂); 5.75 (s, 2H, CH₂-Py); 6.54 (bs, 1H, Ar); 6.97-7.00 (m, 1H, Ar); 7.39-7.41 (m, 1H, Ar); 7.46 (d, J 8.3 Hz, 1H, Ar); 7.63-7.70 (m, 2H, Ar); 7.77-7.81 (m, 1H, Ar); 7.84-7.90 (m, 1H, Ar); 8.38 (s, 1H, Ar); 8.53 (d, J 8.3 Hz, 1H, Ar). 2 protons not observed.

M/Z (M[³⁵Cl]+H)⁺: 641.3.

### Compound 507: 2-(chloromethyl)-3-methylpyridine

To a solution of (3-methylpyridin-2-yl)methanol (100 mg, 1.0 equiv) in DCM (4.0 mL) was added thionyl chloride (4.0 mL). The reaction was stirred at 25° C. for 16 hours. The reaction mixture was concentrated to dryness, the residue was dissolved in water then basified with NaOH 1N aq. until pH=10. The aqueous layer was extracted with DCM (2×40 mL). The organic layer was dried over magnesium sulfate then concentrated to dryness to obtain Compound 507 (100 mg, 87%) as an orange oil.

M/Z (M[³⁵Cl]+H)⁺: 141.9.

### Compound 508: 6-chloro-3-(4-chloro-3-fluorophenyl)-1-((3-methylpyridin-2-yl)methyl)-1H-pyrrolo[2,3-b]pyridine

Compound 508 was prepared according to general procedure (XIII) in NMP from Compound 9 (182 mg) and Compound 507 (140 mg, 1.5 equiv). The reaction mixture was stirred for 45 minutes. The crude was purified by flash chromatography (CyHex 100% to CyHex/EtOAc 80:20) to obtain Compound 508 (141 mg, 56%) as a yellow oil.

M/Z (M[³⁵Cl]₂+H)⁺: 386.2.

### Compound 509: methyl 6-(4-(3-(4-chloro-3-fluorophenyl)-1-((3-methylpyridin-2-yl)methyl)-1H-pyrrolo[2,3-b]pyridine-6-carbonyl)-3,3-dimethylpiperazin-1-yl)-2,4-dimethylnicotinate

Compound 509 was prepared according to general procedure (XXX) starting from Compound 508 (140 mg) and Compound 95 (227 mg, 2.0 equiv). The crude was purified by flash chromatography (CyHex 100% to CyHex/EtOAc 50:50) to obtain Compound 509 (105 mg, 44%) as a yellow oil.

M/Z (M[³⁵Cl]+H)⁺: 655.4.

### Example 193: 6-(4-(3-(4-chloro-3-fluorophenyl)-1-((3-methylpyridin-2-yl)methyl)-1H-pyrrolo[2,3-b]pyridine-6-carbonyl)-3,3-dimethylpiperazin-1-yl)-2,4-dimethylnicotinic acid hydrochloride

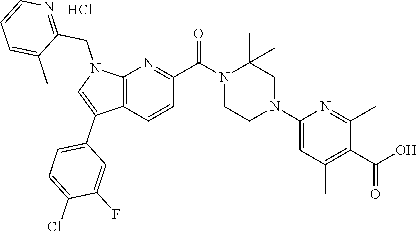 [SMILES: Cc1cccnc1Cn1cc(-c2ccc(Cl)c(F)c2)c2ccc(C(=O)N3CCN(c4cc(C)c(C(=O)O)c(C)n4)CC3(C)C)nc21 ; Cl]

Example 193 was prepared according to general procedure (XIb) starting from Compound 509 (105 mg). The crude was purified by preparative HPLC (20% to 60% ACN/H₂O) and freeze-dried from HCl 1M (5 mL) and water (100 mL) to obtain Example 193 (60 mg, 55%) as a white solid.

¹H-NMR (CD₃OD, 300 MHz) δ: 1.70 (s, 6H, 2 CH₃ of piperazine); 2.52 (s, 3H, CH₃—Ar); 2.57 (s, 3H, CH₃—Ar); 2.67 (s, 3H, CH₃—Ar); 3.70-3.74 (m, 2H, CH₂); 3.87-3.81 (m, 2H, CH₂); 4.00 (s, 2H, CH₂); 5.94 (s, 2H, CH₂—Py); 7.03 (s, 1H, Ar); 7.50-7.63 (m, 4H, Ar); 7.71-7.76 (m, 1H, Ar); 8.03 (s, 1H, Ar); 8.22-8.25 (m, 1H, Ar); 8.48-8.52 (m, 2H, Ar). 2 protons not observed.

M/Z (M[³⁵Cl]+H)⁺: 641.4.

### Example 194: (6-(4-(1-(tert-butyl)-3-(4-chloro-3-fluorophenyl)-1H-pyrrolo[2,3-b]pyridine-6-carbonyl)-3,3-dimethylpiperazin-1-yl)pyridin-3-yl)phosphonic acid

### & Example 195: ethyl hydrogen (6-(4-(1-(tert-butyl)-3-(4-chloro-3-fluorophenyl)-1H-pyrrolo[2,3-b]pyridine-6-carbonyl)-3,3-dimethylpiperazin-1-yl)pyridin-3-yl)phosphonate

To a solution of Example 178 (100 mg, 1 equiv) in DCM (3 mL) at 0° C. was added dropwise bromotrimethylsilane (520 μL, 26 equiv). The reaction was stirred at 25° C. for 5 hours. EtOH (10 mL) was added and the mixture was evaporated to dryness. The crude was purified by preparative HPLC (Column B, 45% to 100% ACN/H₂O), then freeze-dried with water to obtain Example 194 (40 mg, 34%) as a white solid and Example 195 (10 mg, 10%) as a white solid.

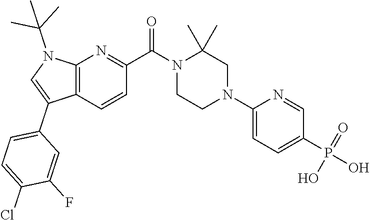 [SMILES: CC1(C)CN(c2ccc(P(=O)(O)O)cn2)CCN1C(=O)c1ccc2c(-c3ccc(Cl)c(F)c3)cn(C(C)(C)C)c2n1]

Example 194: ¹H-NMR (DMSO-d₆, 300 MHz) δ: 1.55 (s, 6H, C(CH₃)₂); 1.82 (s, 9H, C(CH₃)₃); 3.55 (t, J 5.2 Hz, 2H, N—CH₂); 3.89 (t, J 5.2 Hz, 2H, N—CH₂); 3.97 (s, 2H, N—CH₂); 6.63 (d, J 8.3 Hz, 1H, Ar); 7.41 (d, J 8.3 Hz, 1H, Ar); 7.59-7.67 (m, 2H, 2 Ar); 7.68-7.75 (m, 1H, Ar); 7.82 (dd, J 11.1 Hz, 1.7 Hz, 1H, Ar); 8.21 (s, 1H, Ar); 8.32 (dd, J 6.4 Hz, 2.1 Hz, 1H, Ar); 8.43 (d, J 8.3 Hz, 1H, Ar). 2 protons not observed.

M/Z (M[³⁵Cl]+H)⁺: 600.5.

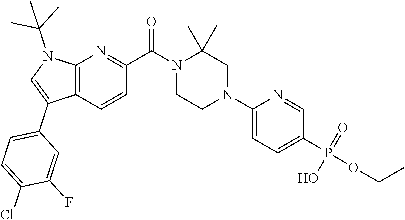 [SMILES: CCOP(=O)(O)c1ccc(N2CCN(C(=O)c3ccc4c(-c5ccc(Cl)c(F)c5)cn(C(C)(C)C)c4n3)C(C)(C)C2)nc1]

Example 195: ¹H-NMR (DMSO-d₆, 300 MHz) δ: 1.12 (t, J 7.1 Hz, 3H, O—CH₂—CH₃); 1.55 (s, 6H, C(CH₃)₂); 1.82 (s, 9H, C(CH₃)₃); 3.55 (t, J 5.2 Hz, 2H, N—CH₂); 3.76 (quint, J 7.1 Hz, 2H, O—CH₂—CH₃); 3.88 (t, J 5.2 Hz, 2H, N—CH₂); 3.96 (s, 2H, N—CH₂); 6.62 (d, J 8.3 Hz, 1H, Ar); 7.41 (d, J 8.3 Hz, 1H, Ar); 7.59-7.67 (m, 2H, 2 Ar); 7.68-7.74 (m, 1H, Ar); 7.82 (dd, J 11.1 Hz, 1.7 Hz, 1H, Ar); 8.21 (s, 1H, Ar); 8.31 (dd, J 6.4 Hz, 2.1 Hz, 1H, Ar); 8.43 (d, J 8.3 Hz, 1H, Ar). 1 proton not observed.

M/Z (M[³⁵Cl]+H)⁺: 628.4.

### Example 196: 8-(1-(tert-butyl)-3-(4-chloro-3-fluorophenyl)-1H-pyrrolo[2,3-b]pyridine-6-carbonyl)-3-methyl-1,3,8-triazaspiro[4.5]decane-2,4-dione

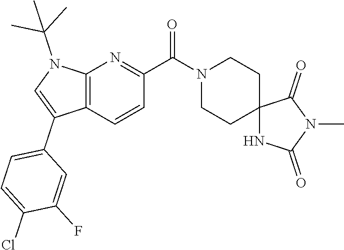 [SMILES: CN1C(=O)NC2(CCN(C(=O)c3ccc4c(-c5ccc(Cl)c(F)c5)cn(C(C)(C)C)c4n3)CC2)C1=O]

Example 196 was prepared according to general procedure (Xa) starting from Compound 78 (43 mg) and 3-methyl-1,3,8-triazaspiro[4.5]decane-2,4-dione (25 mg, 1.1 equiv), and using N,N-diisopropylethylamine (3.0 equiv). The crude was purified by flash chromatography (Interchim® 20 μm, CyHex 100% to EtOAc 100% then to EtOAc/MeOH 90:10) then triturated with MeOH to obtain Example 196 (12 mg, 19%) as a white solid.

¹H-NMR (DMSO-d₆, 300 MHz) δ: 1.58-1.75 (m, 2H, CH₂); 1.79 (s, 9H, C(CH₃)₃); 1.86-2.00 (m, 2H, CH₂); 2.84 (s, 3H, N—CH₃); 3.36-3.41 (m, 1H, N—CHₐH_{b}); 3.45-3.56 (m, 1H, N—CHₐH_{b}); 3.96-4.05 (m, 1H, N—CHₐH_{b}); 4.31-4.40 (m, 1H, N—CHₐH_{b}); 7.46 (d, J 8.3 Hz, 1H, Ar); 7.60-7.67 (m, 2H, 2 Ar); 7.82 (dd, J 11.1 Hz, 1.7 Hz, 1H, Ar); 8.22 (s, 1H, Ar); 8.45 (d, J 8.3 Hz, 1H, Ar); 8.90 (s, 1H, CO—NH).

M/Z (M[³⁵Cl]+H)⁺: 512.4.

### Example 197: 8-(1-(tert-butyl)-3-(4-chloro-3-fluorophenyl)-1H-pyrrolo[2,3-b]pyridine-6-carbonyl)-1,3-dimethyl-1,3,8-triazaspiro[4.5]decane-2,4-dione

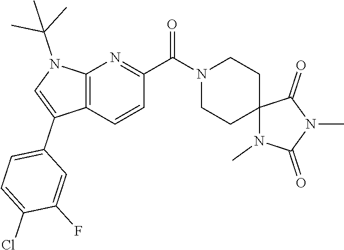 [SMILES: CN1C(=O)N(C)C2(CCN(C(=O)c3ccc4c(-c5ccc(Cl)c(F)c5)cn(C(C)(C)C)c4n3)CC2)C1=O]

Example 197 was prepared according to general procedure (Xa) starting from Compound 78 (43 mg) and 1,3-dimethyl-1,3,8-triazaspiro[4.5]decane-2,4-dione (27 mg, 1.1 equiv), and using N,N-diisopropylethylamine (3.0 equiv). The crude was purified by flash chromatography (Interchim® 20 μm, CyHex 100% to CyHex/EtOAc 30:70) to obtain Example 197 (31 mg, 48%) as a white solid.

¹H-NMR (DMSO-d₆, 300 MHz) δ: 1.68-1.89 (m, 11H, CH₂+C(CH₃)₃); 1.98-2.10 (m, 2H, CH₂); 2.80 (s, 3H, N—CH₃); 2.87 (s, 3H, N—CH₃); 3.39-3.50 (m, 1H, N—CHₐH_{b}); 3.68-3.80 (m, 1H, N—CHₐH_{b}); 4.02-4.11 (m, 1H, N—CHₐH_{b}); 4.55-4.64 (m, 1H, N—CHₐH_{b}); 7.50 (d, J 8.3 Hz, 1H, Ar); 7.60-7.68 (m, 2H, 2 Ar); 7.82 (dd, J 11.1 Hz, 1.7 Hz, 1H, Ar); 8.22 (s, 1H, Ar); 8.45 (d, J 8.3 Hz, 1H, Ar).

M/Z (M[³⁵Cl]+H)⁺: 526.4.

### Compound 510: tert-butyl 4-(benzylamino)-3-methylpiperidine-1-carboxylate

To a suspension of tert-butyl 3-methyl-4-oxopiperidine-1-carboxylate (600 mg, 1 equiv) in THF (20 mL) was added benzylamine (307 μL, 1.0 equiv) and sodium triacetoxyborohydride (1.19 g, 2.0 equiv). The reaction was stirred at 25° C. for 22 hours. The reaction mixture was quenched with NH₄Cl sat. aq. (50 mL) then extracted with EtOAc (50 mL). The organic layer was washed with brine (50 mL), dried over magnesium sulfate then concentrated to dryness. The crude was purified by flash chromatography (Merck 60®, DCM 100% to DCM/MeOH 85:15) to obtain Compound 510 (550 mg, 64%).

M/Z (M+H)⁺: 305.2

### Compound 511: tert-butyl 4-amino-3-methylpiperidine-1-carboxylate

To a solution of Compound 510 (543 mg, 1.0 equiv) in MeOH (30 mL) was added palladium on carbon (95 mg, 10% Wt, 0.05 equiv). The reaction was stirred hydrogen atmosphere (1 atm) at 25° C. for 72 hours. The reaction mixture was filtered through Celite® and the filtrate was concentrated to dryness to obtain Compound 511 (420 mg) as a colorless oil.

M/Z (M+H)⁺: 215.2.

### Compound 512: methyl 6-((1-(tert-butoxycarbonyl)-3-methylpiperidin-4-yl)amino)-2,4-dimethylnicotinate

Compound 512 was prepared according to general procedure (XXI) starting from methyl 6-chloro-2,4-dimethylnicotinate (354 mg) and Compound 511 (380 mg). The crude residue was purified by flash chromatography (CyHex 100% to EtOAc 100% then to EtOAc/MeOH 80:20) to obtain Compound 512 (296 mg) as a yellow oil.

M/Z (M+H)⁺: 378.2.

### Compound 513: methyl 2,4-dimethyl-6-((3-methylpiperidin-4-yl)amino)nicotinate hydrochloride

Compound 513 was obtained (65 mg, 98%) as a beige solid according to general procedure (XVIIIc) starting from Compound 512 (80 mg).

M/Z (M+H)⁺: 278.2.

### Compound 514: methyl 6-((1-(1-(tert-butyl)-3-(4-chloro-3-fluorophenyl)-1H-pyrrolo[2,3-b]pyridine-6-carbonyl)-3-methylpiperidin-4-yl)amino)-2,4-dimethylnicotinate

Compound 514 was prepared according to general procedure (Xa) starting from Compound 78 (49 mg) and Compound 513 (65 mg, 1.2 equiv) and using N,N-diisopropylethylamine (4.0 equiv). The crude residue was purified by flash chromatography (CyHex 100% to CyHex/EtOAc 50:50) to obtain Compound 514 (94 mg, 99%) as a white solid.

M/Z (M[³⁵Cl]+H)⁺: 606.5

### Example 198: 6-((1-(1-(tert-butyl)-3-(4-chloro-3-fluorophenyl)-1H-pyrrolo[2,3-b]pyridine-6-carbonyl)-3-methylpiperidin-4-yl)amino)-2,4-dimethylnicotinic acid

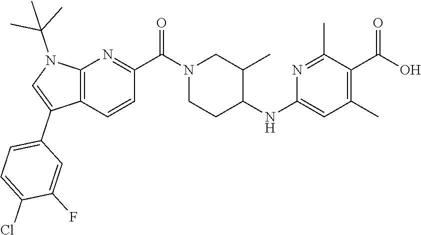 [SMILES: Cc1cc(NC2CCN(C(=O)c3ccc4c(-c5ccc(Cl)c(F)c5)cn(C(C)(C)C)c4n3)CC2C)nc(C)c1C(=O)O]

Example 198 was prepared according to general procedure (XIb) starting from Compound 514 (80 mg). The crude was purified by flash chromatography (DCM 100% to DCM/MeOH 80:20) then freeze-dried in EtOH/water to obtain Example 198 (67 mg, 73%) as a beige solid.

¹H-NMR (DMSO-d₆, 300 MHz) δ: 0.66 (d, 1.5H, J 6.9 Hz, one diastereoisomer of CH₃); 0.91 (d, 1.6H, J 6.9 Hz, other diastereoisomer of CH₃); 1.57-1.74 (m, 2H, CH₂); 1.63-1.80 (m, 10H, CH, C(CH₃)₃); 2.12 (s, 3H, CH₃); 2.25 (s, 3H, CH₃); 3.47-3.63 (m, 2H, CH₂); 3.66-3.90 (m, 2H, CH₂); 4.14-4.22 (m, 1H, CH); 6.10-6.17 (m, 2H, NH, Ar); 77.39 (t, 1H, J 7.5 Hz, Ar); 7.59-7.67 (m, 2H, Ar); 7.71 (dd, 1H, J 11.4, 2.1 Hz, Ar); 8.19 (s, 1H, Ar); 8.44 (d, 1H, J 8.4 Hz, Ar). COOH signal not observed

M/Z (M[³⁵Cl]+H)⁺: 592.5

### Compound 515: methyl 6-((1-(tert-butoxycarbonyl)-3-methylpiperidin-4-yl)(methyl)amino)-2,4-dimethylnicotinate

Compound 515 was prepared according to general procedure (XIII) in DMF from Compound 512 (296 mg) and iodomethane (171 μL, 3.5 equiv). The reaction mixture was stirred for 20 hours. The crude was purified by flash chromatography (Merck 60®, CyHex 100% to CyHex/EtOAc 70:30) to obtain Compound 515 (190 mg) as a white solid.

M/Z (M+H)⁺: 392.3.

### Compound 516: methyl 2,4-dimethyl-6-(methyl(3-methylpiperidin-4-yl)amino)nicotinate hydrochloride

Compound 516 was obtained (120 mg) as a white solid according to general procedure (XVIIIc) starting from Compound 515 (190 mg).

M/Z (M+H)⁺: 292.2.

### Compound 517: methyl 6-((1-(1-(tert-butyl)-3-(4-chloro-3-fluorophenyl)-1H-pyrrolo[2,3-b]pyridine-6-carbonyl)-3-methylpiperidin-4-yl)(methyl)amino)-2,4-dimethylnicotinate

Compound 517 was prepared according to general procedure (Xa) starting from Compound 78 (63 mg) and Compound 516 (60 mg, 1.0 equiv) and using N,N-diisopropylethylamine (4.0 equiv). The crude residue was purified by flash chromatography (Merck 60®, CyHex 100% to CyHex/EtOAc 50:50) to obtain Compound 517 (100 mg, 88%) as a white solid.

M/Z (M[³⁵Cl]+H)⁺: 620.5

### Example 199: 6-((1-(1-(tert-butyl)-3-(4-chloro-3-fluorophenyl)-1H-pyrrolo[2,3-b]pyridine-6-carbonyl)-3-methylpiperidin-4-yl)(methyl)amino)-2,4-dimethylnicotinic acid

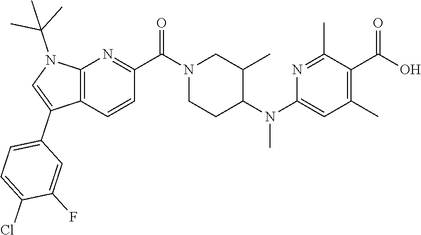 [SMILES: Cc1cc(N(C)C2CCN(C(=O)c3ccc4c(-c5ccc(Cl)c(F)c5)cn(C(C)(C)C)c4n3)CC2C)nc(C)c1C(=O)O]

Example 199 was prepared according to general procedure (XIb) starting from Compound 517 (100 mg). The crude was purified by flash chromatography (Merck 60®, CyHex 100% to EtOAc 100% then to EtOAc/MeOH: 75:25) then further purified by preparative HPLC (Column B, 20% to 100% ACN/H₂O) and freeze-dried to obtain Example 199 (60 mg, 61%) as a white solid.

Mixture of 2 couples of diastereoisomers (8:2) and 2 rotamers

¹H-NMR (DMSO-d₆, 300 MHz) δ: 0.57-0.97 (m, 3H, CH₃); 1.52-1.67 (m, 1H, CH); 1.95-2.06 (m, 1H, CH); 2.23-2.27 (m, 3H, CH₃); 2.35-2.39 (m, 3H, CH₃); 2.84-2.95 (m, 3H, CH₃); 3.08-3.23 (m, 1H, CH); 3.48-3.69 (m, 1H, CH); 4.05-4.47 (m, 1H, CH); 4.63-4.69 (m, 1H, CH); 6.32-6.40 (m, 1H, Ar); 7.34-7.47 (m, 1H, Ar); 7.59-7.64 (m, 2H, Ar); 7.79-7.84 (m, 1H, Ar); 8.18-8.22 (m, 1H, Ar); 8.40-8.46 (m, 1H, Ar); 12.64 (bs, 1H, CO₂H).

M/Z (M[³⁵Cl]+H)⁺: 606.5.

### Compound 518: tert-butyl 6,6-dimethyl-2,4-dioxo-1,3,8-triazaspiro[4.5]decane-8-carboxylate

To a suspension of tert-butyl 3,3-dimethyl-4-oxopiperidine-1-carboxylate (200 mg, 1.0 equiv) in a mixture Ethanol (28 mL) and Water (8 mL) were added sodium cyanide (86 mg, 2.0 equiv) and ammonium carbonate (1.69 g, 20 equiv). The reaction was heated at 80° C. for 12 hours. The reaction mixture was partially concentrated under reduced pressure, and the aqueous residue was extracted thrice with EtOAc (3×25 mL), dried over magnesium sulfate then concentrated to dryness to obtain Compound 518 (264 mg) as a beige solid.

M/Z (M-^{t}Bu+H)⁺: 242.1

### Compound 519: 6,6-dimethyl-1,3,8-triazaspiro[4.5]decane-2,4-dione hydrochloride

Compound 519 was obtained (220 mg) as a beige solid according to general procedure (XVIIIc) starting from Compound 518 (279 mg).

M/Z (M+H)⁺: 198.0.

### Example 200: 8-(1-(tert-butyl)-3-(4-chloro-3-fluorophenyl)-1H-pyrrolo[2,3-b]pyridine-6-carbonyl)-6,6-dimethyl-1,3,8-triazaspiro[4.5]decane-2,4-dione

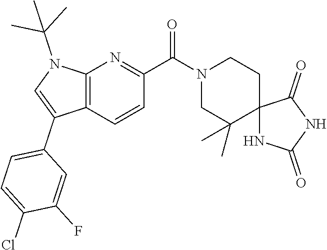 [SMILES: CC(C)(C)n1cc(-c2ccc(Cl)c(F)c2)c2ccc(C(=O)N3CCC4(NC(=O)NC4=O)C(C)(C)C3)nc21]

Example 200 was prepared according to general procedure (Xa) starting from Compound 78 (75 mg) and Compound 519 (66 mg, 1.3 equiv), and using N,N-diisopropylethylamine (4.0 equiv). The crude was purified by flash chromatography (CyHex 100% to EtOAc 100%) then further purified by preparative HPLC (40% to 80% ACN/H₂O) and freeze-dried with MeOH/water to obtain Example 200 (69 mg, 61%) as a white solid.

¹H-NMR (DMSO-d₆, 300 MHz, 60° C.) δ: 0.66-1.10 (m, 6H, 2*CH₃); 1.81-1.97 (m, 11H, CH₂, C(CH₃)₃); 3.52-4.12 (m, 4H, 2*CH₂); 7.42 (d, 1H, J 8.1 Hz, Ar); 7.57-7.65 (m, 2H, Ar); 7.77 (dd, 1H, J 10.8, 1.8 Hz, Ar); 7.99-8.14 (m, 1H, NH); 8.16 (s, 1H, Ar); 8.40 (d, 1H, J 8.1 Hz, Ar); 10.5 (s, 1H, NH).

M/Z (M[³⁵Cl]+H)⁺: 526.4

### Compound 520: tert-butyl 6,6-difluoro-2,4-dioxo-1,3,8-triazaspiro[4.5]decane-8-carboxylate

To a suspension of tert-butyl 3,3-difluoro-4-oxopiperidine-1-carboxylate (200 mg, 1.0 equiv) in a mixture Ethanol (28 mL) and Water (8 mL) were added sodium cyanide (83 mg, 2.0 equiv) and ammonium carbonate (1.63 g, 20 equiv). The reaction was heated at 80° C. for 12 hours. The reaction mixture was partially concentrated under reduced pressure, and the aqueous residue was extracted thrice with EtOAc (3×25 mL), dried over magnesium sulfate then concentrated to dryness to obtain Compound 520 (270 mg) as a beige solid.

M/Z (M-Boc+H)⁺: 206.0

### Compound 521: 6,6-difluoro-1,3,8-triazaspiro[4.5]decane-2,4-dione hydrochloride

Compound 521 was obtained (230 mg) as a beige solid according to general procedure (XVIIIc) starting from Compound 520 (286 mg).

M/Z (M+H)⁺: 206.0.

### Example 201: 8-(1-(tert-butyl)-3-(4-chloro-3-fluorophenyl)-1H-pyrrolo[2,3-b]pyridine-6-carbonyl)-6,6-difluoro-1,3,8-triazaspiro[4.5]decane-2,4-dione

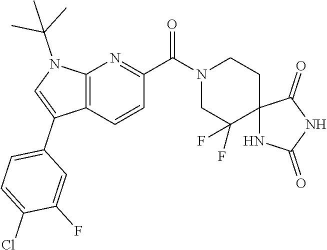 [SMILES: CC(C)(C)n1cc(-c2ccc(Cl)c(F)c2)c2ccc(C(=O)N3CCC4(NC(=O)NC4=O)C(F)(F)C3)nc21]

Example 201 was prepared according to general procedure (Xa) starting from Compound 78 (75 mg) and Compound 521 (66 mg, 1.3 equiv), and using N,N-diisopropylethylamine (4.0 equiv). The crude was purified by flash chromatography (CyHex 100% to EtOAc 100%) then further purified by preparative HPLC (40% to 80% ACN/H₂O) and freeze-dried with MeOH/water to obtain Example 201 (55 mg, 48%) as a white solid.

¹H-NMR (DMSO-d₆, 300 MHz) δ: 1.80 (s, 9H, C(CH₃)₃); 1.93-1.23 (m, 1H, CH₂); 2.13-2.25 (m, 1H, CH₂); 3.42-3.64 (m, 3H, 1.5*CH₂); 4.52-4.68 (m, 1H, CH₂); 7.52 (m, 1H, Ar); 7.60-7.68 (m, 2H, Ar); 7.79 (dd, 1H, J 11.0, 0.9 Hz, Ar); 8.26 (d, 1H, J 11.0, Ar); 8.47 (d, 1H, J 8.4 Hz, Ar); 8.69-8.76 (m, 1H, NH); 11.2 (s, 1H, NH).

M/Z (M[³⁵Cl]+H)⁺: 534.4

### Compound 522: tert-butyl 2-oxo-1,8-diazaspiro[4.5]decane-8-carboxylate

To a solution of 1-t-butoxycarbonyl-4-aminopiperidine (117 mg, 1.0 equiv) in ACN (2 mL) was added methyl acrylate (52.4 μL, 1.0 equiv), 4-CzlPN (1.37 mg, 0.003 equiv) and tetrabutylammonium azide (3.30 mg, 0.02 equiv). The reaction was irradiated at 450 nm using PennPhD at 30° C. for 18 hours. The reaction mixture was quenched with water (20 mL) then extracted with EtOAc (20 mL). The organic layer was washed with brine (20 mL), dried over magnesium sulfate then concentrated to dryness. The crude was purified by flash chromatography (DCM 100% to DCM/MeOH 90:10) to obtain Compound 522 (55 mg, 37%) as a colorless oil.

M/Z (M+H)⁺: 255.2.

### Compound 523: 1,8-diazaspiro[4.5]decan-2-one hydrochloride

Compound 523 was obtained (37 mg, 99%) as a white solid according to general procedure (XVIIIc) starting from Compound 522 (50 mg).

M/Z (M+H)⁺: 155.2.

### Example 202: 8-(1-(tert-butyl)-3-(4-chloro-3-fluorophenyl)-1H-pyrrolo[2,3-b]pyridine-6-carbonyl)-1,8-diazaspiro[4.5]decan-2-one

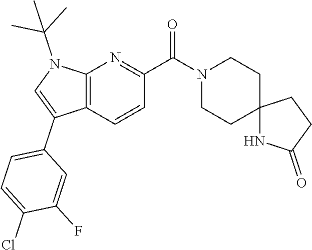 [SMILES: CC(C)(C)n1cc(-c2ccc(Cl)c(F)c2)c2ccc(C(=O)N3CCC4(CCC(=O)N4)CC3)nc21]

Example 202 was prepared according to general procedure (Xc) starting from Compound 78 (38 mg, 1.2 equiv) and Compound 523 (17 mg), and using N,N-diisopropylethylamine (4.0 equiv). The crude was purified by flash chromatography (DCM 100% to DCM/MeOH 80:20) then further purified by preparative HPLC (Column B, 20% to 100% ACN/H₂O) and freeze-dried to obtain Example 202 (12 mg, 27%) as a white solid.

¹H-NMR (DMSO-d₆, 300 MHz) δ: 1.59-1.72 (m, 4H, 2*CH₂); 1.79 (s, 9H, C(CH₃)₃); 1.86-1.95 (m, 2H, CH₂); 2.18-2.25 (m, 2H, CH₂); 3.50-3.89 (m, 4H, CH, 2*CH₂); 7.41 (d, 1H, J 8.4 Hz, Ar); 7.59-7.67 (m, 2H, Ar); 7.82 (dd, 1H, J 11.0, 1.8 Hz, Ar); 8.11 (s, 1H, NH); 8.21 (s, 1H, Ar); 8.42 (d, 1H, J 8.4 Hz, Ar).

M/Z (M[³⁵Cl]+H)⁺: 483.3

### Compound 524: methyl 2-(1-(1H-indole-6-carbonyl)piperidin-4-yl)acetate

Compound 524 was prepared according to general procedure (Xa) starting from 1H-indole-6-carboxylic acid (161 mg, 1.0 equiv) and methyl 2-(piperidin-4-yl)acetate hydrochloride (194 mg, 1.0 equiv) and using N,N-diisopropylethylamine (4.0 equiv). The crude residue was purified by flash chromatography (Merck 60®, CyHex 100% to EtOAc 100%) to obtain Compound 524 (240 mg) as a white solid.

M/Z (M+H)⁺: 301.2

### Compound 525: methyl 2-(1-(3-bromo-1H-indole-6-carbonyl)piperidin-4-yl)acetate

To a solution of Compound 524 (220 mg, 1.0 equiv) in DMF (10 mL) at 0° C. was added dropwise a solution of N-bromosuccinimide (130 mg, 1.0 equiv) in DMF (2 mL). The reaction was stirred at 0° C. for 2 hours. The reaction mixture was quenched with water (100 mL) then extracted with EtOAc (2×50 mL). The organic layer was washed with 10% aq. Na₂S₂O₃ (50 mL), water (15 mL), dried over magnesium sulfate then concentrated to dryness. The crude was purified by flash chromatography (Merck 60®, DCM 100% to DCM/MeOH 90:10) to obtain Compound 525 as a pink oil (191 mg, 50% over 2 steps). M/Z (M[⁸¹Br]+H)⁺: 381.2.

### Compound 526: methyl 2-(1-(3-bromo-1-isobutyl-1H-indole-6-carbonyl)piperidin-4-yl)acetate

Compound 526 was prepared according to general procedure (XIII) in DMF starting from Compound 525 (329 mg), isobutyl iodide (232 μL, 2.3 equiv) and sodium hydride (87 mg, 60 Wt. %, 2.5 equiv). The reaction mixture was heated at 80° C. for 18 hours. The crude was purified by flash chromatography (Merck 60®, CyHex 100% to CyHex/EtOAc 60:40) to obtain Compound 526 (253 mg, 67%) as a colorless oil.

M/Z (M[⁸¹Br]+H)⁺: 437.3

### Example 203: methyl 2-(1-(3-(4-chloro-3-fluorophenyl)-1-isobutyl-1H-indole-6-carbonyl)piperidin-4-yl)acetate

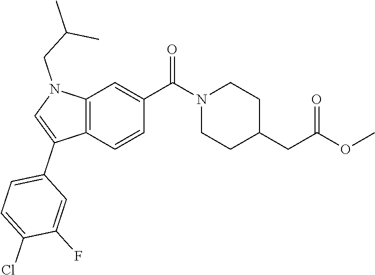 [SMILES: COC(=O)CC1CCN(C(=O)c2ccc3c(-c4ccc(Cl)c(F)c4)cn(CC(C)C)c3c2)CC1]

Example 203 was prepared according to general procedure (XVIIa) starting from Compound 526 (120 mg) and (4-chloro-3-fluorophenyl)boronic acid (58 mg, 1.2 equiv). The crude was purified by flash chromatography (Merck 60®, CyHex 100% to CyHex/EtOAc 20:80), then further purified by flash chromatography (Interchim® 20 μm, CyHex 100% to CyHex/EtOAc 20:80) and freeze-dried in ACN/water to obtain Example 203 (90 mg, 67%) as a white solid.

¹H-NMR (DMSO-d₆, 300 MHz) δ: 0.87 (d, J 6.6 Hz, 6H, (CH₃)₂); 1.11-1.26 (m, 2H, CH₂); 1.55-1.75 (m, 2H, CH₂); 1.92-2.02 (m, 1H, CH); 2.10-2.18 (m, 1H, CH); 2.29 (d, J 6.6 Hz, 2H, CH₂); 2.80-3.10 (m, 2H, CH₂); 3.28-3.32 (m, 1H, CH); 3.58 (s, 3H, CH₃); 3.60-3.70 (m, 1H, CH); 4.06 (d, J 7.2 Hz, 2H, CH₂); 4.35-4.50 (m, 1H, CH); 7.13 (dd, J 1.5, 8.4 Hz, 1H, Ar); 7.55-7.61 (m, 3H, Ar); 7.69 (dd, J 1.5, 11.4 Hz, 1H, Ar); 7.91 (d, J 8.4 Hz, 1H, Ar); 8.02 (s, 1H, Ar).

M/Z (M[³⁵Cl]+H)⁺: 485.4.

### Example 204: 2-(1-(3-(4-chloro-3-fluorophenyl)-1-isobutyl-1H-indole-6-carbonyl)piperidin-4-yl)acetic acid

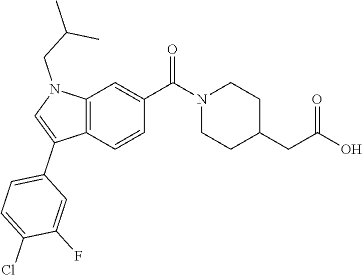 [SMILES: CC(C)Cn1cc(-c2ccc(Cl)c(F)c2)c2ccc(C(=O)N3CCC(CC(=O)O)CC3)cc21]

Example 204 was prepared according to general procedure (IXa) starting from Example 203 (80 mg). The crude was purified by preparative HPLC (50% to 90% ACN/H₂O), then freeze-dried with water to obtain Example 204 (40 mg, 51%) as a white solid.

¹H-NMR (DMSO-d₆, 300 MHz) δ: 0.87 (d, J 6.9 Hz, 6H, (CH₃)₂); 1.10-1.24 (m, 2H, CH₂); 1.56-1.86 (m, 2H, CH₂); 1.93-2.09 (m, 1H, CH); 2.11-2.16 (m, 1H, CH); 2.19 (d, J 6.9 Hz, 2H, CH₂); 2.76-3.08 (m, 2H, CH₂); 3.59-3.77 (m, 1H, CH); 4.06 (d, J 7.0 Hz, 2H, CH₂); 4.36-4.55 (m, 1H, CH); 7.13 (dd, J 1.5, 8.4 Hz, 1H, Ar); 7.55-7.64 (m, 3H, Ar); 7.68 (dd, J 1.5, 11.4 Hz, 1H, Ar); 7.91 (d, J 8.4 Hz, 1H, Ar); 7.99 (s, 1H, Ar); 12.11 (bs, 1H, CO₂H).

M/Z (M[³⁵Cl]+H)⁺: 471.2.

### Compound 527: 6-chloro-3-(4-chloro-3-fluorophenyl)-2-(trifluoromethyl)-1H-pyrrolo[2,3-b]pyridine

To a suspension of Compound 9 (170 mg, 1 equiv) in ACN (4.0 mL) was added tert-Butyl hydroperoxide in water (228 μL, 80% Wt, 3.0 equiv) and sodium trifluoromethylsulfinate (189 mg, 2.0 equiv). The reaction was heated at 130° C. for 66 hours. The reaction mixture was quenched with water (50 mL) then extracted with EtOAc (50 mL). The organic layer was washed with brine (50 mL), dried over magnesium sulfate then concentrated to dryness. The crude was purified by flash chromatography (CyHex 100% to CyHex/EtOAc 80:20) to obtain Compound 527 (60 mg, 28%) as a yellow solid.

M/Z (M[³⁵Cl]₂+H)⁺: 349.0.

### Compound 528: 6-chloro-3-(4-chloro-3-fluorophenyl)-1-isobutyl-2-(trifluoromethyl)-1H-pyrrolo[2,3-b]pyridine

Compound 528 was prepared according to general procedure (XIII) in DMF from Compound 527 (130 mg) and 1-iodo-2-methylpropane (62 μL, 1.5 equiv). The reaction mixture was stirred at 80° C. for 20 hours. The crude was purified by flash chromatography (CyHex 100% to CyHex/EtOAc 95:05) to obtain Compound 528 (90 mg, 60%) as a yellow oil.

M/Z (M[³⁵Cl]₂+H)⁺: 405.1.

### Compound 529: methyl 6-(4-(3-(4-chloro-3-fluorophenyl)-1-isobutyl-2-(trifluoromethyl)-1H-pyrrolo[2,3-b]pyridine-6-carbonyl)-3,3-dimethylpiperazin-1-yl)-2,4-dimethylnicotinate

Compound 529 was prepared according to general procedure (XXX) starting from Compound 528 (90 mg) and Compound 95 (139 mg, 2.0 equiv). The crude was purified by flash chromatography (CyHex 100% to CyHex/EtOAc 70:30) to obtain Compound 529 (95 mg, n.d.) as a beige solid.

M/Z (M[³⁵Cl]+H)⁺: 674.3.

### Example 205: 6-(4-(3-(4-chloro-3-fluorophenyl)-1-isobutyl-2-(trifluoromethyl)-1H-pyrrolo[2,3-b]pyridine-6-carbonyl)-3,3-dimethylpiperazin-1-yl)-2,4-dimethyinicotinic acid

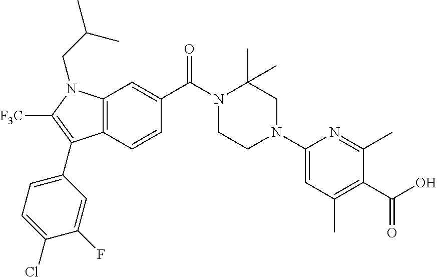 [SMILES: Cc1cc(N2CCN(C(=O)c3ccc4c(-c5ccc(Cl)c(F)c5)c(C(F)(F)F)n(CC(C)C)c4c3)C(C)(C)C2)nc(C)c1C(=O)O]

Example 205 was prepared according to general procedure (XIb) starting from Compound 529 (95 mg). The crude was purified by preparative HPLC (50% to 90% ACN/H₂O), then freeze-dried in water to obtain Example 205 (45 mg, 31% over 2 steps) as a white solid.

¹H-NMR (DMSO-d₆, 300 MHz) δ: 0.91 (d, J 6.6 Hz, 6H, 2*N—CH₂—CH—CH₃); 1.53 (s, 6H, 2*CH₃); 2.26 (s, 3H, Ar—CH₃); 2.37 (s, 4H, N—CH₂—CH—CH₃+Ar—CH₃); 3.52-3.60 (m, 2H, N—CH₂); 3.72-3.82 (m, 2H, N—CH₂); 3.88-3.96 (m, 2H, N—CH₂); 4.31 (d, J 7.5 Hz, 2H, N—CH₂—CH—CH₃); 6.34 (s, 1H, Ar); 7.28-7.35 (m, 1H, Ar); 7.46 (d, J 8.3 Hz, 1H, Ar); 7.56 (dd, J 10.1, 1.8 Hz, 1H, Ar); 7.73 (m, 1H, Ar); 8.05 (d, J 8.3 Hz, 1H, Ar); 12.71 (bs, 1H, COOH).

M/Z (M[³⁵Cl]+H)⁺: 660.3.

### Compound 530: methyl 1-(4-chloro-3-fluorophenyl)-1H-indole-5-carboxylate

To a solution of methyl 1H-indole-5-carboxylate (250 mg, 1.0 equiv) in ACN (5.0 mL) was added 1-chloro-2-fluoro-4-iodo-benzene (219 μL, 1.2 equiv), potassium carbonate (493 mg, 2.5 equiv), (1R,2R)—N1,N2-dimethylcyclohexane-1,2-diamine (23 μL, 0.1 equiv) and copper(I) iodide (54 mg, 0.2 equiv). The reaction was heated at 82° C. for 18 hours. The reaction mixture was quenched with NH₄Cl sat. aq. (30 mL) then extracted with EtOAc (30 mL). The organic layer was washed with brine (30 mL), dried over magnesium sulfate then concentrated to dryness. The crude was purified by flash chromatography (Merck 60®, CyHex 100% to CyHex/EtOAc 50:50) to obtain Compound 530 (330 mg, 76%) as a white solid.

M/Z (M[³⁵Cl]+H)⁺: 304.1.

### Compound 531: 3-acetyl-1-(4-chloro-3-fluorophenyl)-1H-indole-5-carboxylic acid

To a solution of acetyl chloride (92 μL, 1.5 equiv) in Nitromethane (10 mL) was added aluminum chloride (685 mg, 6.0 equiv). The reaction mixture was stirred at 25° C. for 1 hour and Compound 530 (260 mg, 1.0 equiv) was added portionwise. The reaction mixture was stirred at 100° C. for 3 hours. The reaction mixture was quenched with HCl 1N aqu. (200 mL) then extracted with EtOAc (3×100 mL). The organic layer was washed with brine (20 mL), dried over magnesium sulfate then concentrated to dryness. The residue was solubilized in a mixture of K₂CO₃ sat. aq. (200 mL) and water 100 mL, washed with EtOAc (2×50 mL). The alkaline solution was slowly acidified with HCl 6N aqu. until pH<3, then extracted with EtOAc (3×100 mL). The organic layer was washed with brine (50 mL), dried over magnesium sulfate then concentrated to dryness to obtain Compound 531 (250 mg) as a brown solid.

M/Z (M[³⁵Cl]+H)⁺: 332.2.

### Compound 532: methyl 2-(1-(3-acetyl-1-(4-chloro-3-fluorophenyl)-1H-indole-5-carbonyl)piperidin-4-yl)acetate

Compound 532 was prepared according to general procedure (Xa) starting from Compound 531 (120 mg) and methyl 2-(piperidin-4-yl)acetate (57 mg, 1.0 equiv), and using N,N-diisopropylethylamine (4.0 equiv). The crude was purified by flash chromatography (Merck 60®, CyHex 100% to EtOAc 100%) to obtain Compound 532 (100 mg) as a brown solid.

M/Z (M[³⁵Cl]+H)⁺: 471.3

### Example 206: 2-(1-(3-acetyl-1-(4-chloro-3-fluorophenyl)-1H-indole-5-carbonyl)piperidin-4-yl)acetic acid

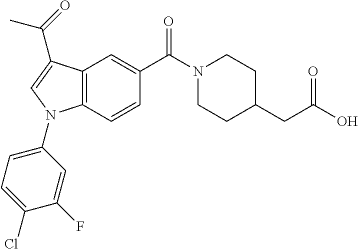 [SMILES: CC(=O)c1cn(-c2ccc(Cl)c(F)c2)c2ccc(C(=O)N3CCC(CC(=O)O)CC3)cc12]

Example 206 was prepared according to general procedure (IXa) starting from Compound 532 (120 mg). The crude was purified by preparative HPLC (30% to 70% ACN/H₂O), then freeze-dried in water to obtain Example 206 (55 mg, 11% over 3 steps) as a yellow solid.

¹H-NMR (DMSO-d₆, 300 MHz) δ: 1.02-1.30 (m, 2H, CH₂); 1.50-1.81 (m, 2H, CH₂); 1.86-2.01 (m, 1H, CH); 2.19 (d, J 6.6 Hz, 2H, CH₂); 2.53 (s, 3H, CH₃); 2.77-3.16 (m, 2H, CH₂); 3.51-3.81 (m, 1H, CH); 4.33-4.58 (m, 1H, CH); 7.32 (dd, J 8.4, 1.2 Hz, 1H, Ar); 7.62-7.65 (m, 2H, Ar); 7.87 (t, J 8.4 Hz, 1H, Ar); 7.94 (dd, J 10.2, 2.4 Hz, 1H, Ar); 8.28 (d, J 1.2 Hz, 1H, Ar); 8.75 (s, 1H, Ar); 12.10 (s, 1H, CO₂H).

M/Z (M[³⁵Cl]+H)⁺: 457.2.

### Compound 533: 1-isopropyl-1,3,8-triazaspiro[4.5]decan-4-one dihydrochloride

Compound 533 was obtained (363 mg, qt.) as a beige solid according to general procedure (XVIIIc) starting from tert-butyl 1-isopropyl-4-oxo-1,3,8-triazaspiro[4.5]decane-8-carboxylate (400 mg).

M/Z (M+H)⁺: 198.1

### Example 207: 8-(1-(tert-butyl)-3-(4-chloro-3-fluorophenyl)-1H-pyrrolo[2,3-b]pyridine-6-carbonyl)-1-isopropyl-1,3,8-triazaspiro[4.5]decan-4-one

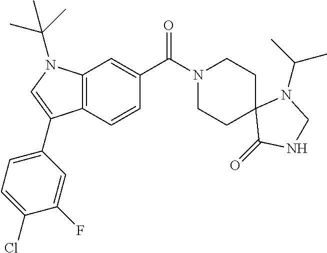 [SMILES: CC(C)N1CNC(=O)C12CCN(C(=O)c1ccc3c(-c4ccc(Cl)c(F)c4)cn(C(C)(C)C)c3c1)CC2]

Example 207 was prepared according to general procedure (Xb) starting from Compound 78 (60 mg, 1.0 equiv) and Compound 533 (47 mg), and using N,N-diisopropylethylamine (5.0 equiv). The crude was purified by flash chromatography (CyHex 100% to EtOAc 100% then to EtOAc/meOH 97/3) then freeze-dried in EtOH/water to obtain Example 207 (50 mg, 55%) as a white solid.

¹H-NMR (DMSO-d₆, 300 MHz) δ: 1.04-1.07 (m, 6H, N—CH—(CH₃)₂); 1.58-1.94 (m, 13H, 2*CH₂+C(CH₃)₃); 3.06-3.17 (m, 1H, N—CH—(CH₃)₂); 3.56-3.64 (m, 1H, N—CHₐH_{b}); 3.70-3.84 (m, 2H, N—CHₐH_{b}+N—CHₐH_{b}); 4.09-4.13 (m, 2H, CH₂); 4.21-4.32 (m, 1H, N—CHₐH_{b}); 7.43 (d, J 8.3 Hz, 1H, Ar); 7.59-7.68 (m, 2H, 2 Ar); 7.82 (dd, J 11.1, 1.7 Hz, 1H, Ar); 8.20 (s, 1H, Ar); 8.28 (s, 1H, NH), 8.43 (d, J 8.3 Hz, 1H, Ar).

M/Z (M[³⁵Cl]+H)⁺: 526.4.

### Compound 534: 4-chloro-5-(piperazin-1-yl)pyridazin-3(2H)-one

To a suspension of 4,5-dichloropyridazin-3-(2H)-one (15.0 g, 1.0 equiv) in a mixture THF (120 mL)/H₂O (30 mL) was added piperazine (13.3 g, 1.7 equiv). The reaction mixture was heated at 80° C. for 18 hours. The precipitate was filtered, washed with MeOH and dried under vacuum to obtained Compound 534 (15.84 g, 81%) as a yellow solid.

Compound 535: 5-(piperazin-1-yl)pyridazin-3(2H)-one

To solution of Compound 534 (18.5 g, 1.0 equiv) in a mixture MeOH (100 mL)/H₂O (100 mL) was added palladium hydroxide 10-20 wt. % on charcoal (500 mg). The reaction mixture was stirred under hydrogen atmosphere (4 bars) for 24 hours. The reaction mixture was filtered over Celite*. The filtrate was concentrated and triturated in MeOH to obtain Compound 535 (10.15 g, 65%) as a white solid.

### Example 208: 5-(4-(1-(tert-butyl)-3-(4-chloro-3-fluorophenyl)-1H-pyrrolo[2,3-b]pyridine-6-carbonyl)piperazin-1-yl)pyridazin-3(2H)-one

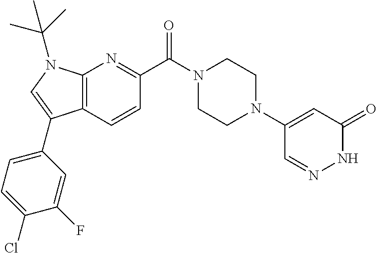 [SMILES: CC(C)(C)n1cc(-c2ccc(Cl)c(F)c2)c2ccc(C(=O)N3CCN(c4cn[nH]c(=O)c4)CC3)nc21]

Example 208 was prepared according to general procedure (Xa) starting from Compound 78 (60 mg) and Compound 535 (37 mg, 1.0 equiv), and using N,N-diisopropylethylamine (4.0 equiv). The precipitate formed was filtered, triturated with CH₂Cl₂, H₂O and Et₂O and dried under vacuum to obtain Example 208 (41 mg, 47%) as a white solid.

¹H-NMR (DMSO-d₆, 300 MHz) δ: 1.81 (m, 9H, C(CH₃)₃); 3.40-3.48 (m, 2H, N—CH₂); 3.48-3.57 (m, 2H, N—CH₂); 3.73-3.83 (m, 4H, 2*N—CH₂); 5.79 (s, 1H, Ar); 7.50 (d, J 8.3 Hz, 1H, Ar); 7.60-7.68 (m, 2H, 2 Ar); 7.83 (dd, J 11.1, 1.7 Hz, 1H, Ar); 7.95-7.97 (m, 1H, Ar); 8.24 (s, 1H, Ar); 8.46 (d, J 8.3 Hz, 1H, Ar); 12.26 (s, 1H, NH).

M/Z (M[³⁵Cl]+H)⁺: 509.4.

### Compound 536: 1-methyl-1,3,8-triazaspiro[4.5]decan-4-one dihydrochloride

Compound 536 was obtained (360 mg) as a beige solid according to general procedure (XVIIIc) starting from tert-butyl 1-methyl-4-oxo-1,3,8-triazaspiro[4.5]decane-8-carboxylate (400 mg).

M/Z (M+H)⁺: 170.0.

### Example 209: 8-(1-(tert-butyl)-3-(4-chloro-3-fluorophenyl)-1H-pyrrolo[2,3-b]pyridine-6-carbonyl)-1-methyl-1,3,8-triazaspiro[4.5]decan-4-one

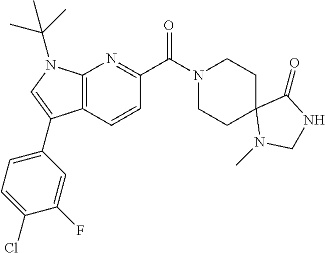 [SMILES: CN1CNC(=O)C12CCN(C(=O)c1ccc3c(-c4ccc(Cl)c(F)c4)cn(C(C)(C)C)c3n1)CC2]

Example 209 was prepared according to general procedure (Xb) starting from Compound 78 (75 mg) and Compound 536 (53 mg, 1.2 equiv), and using N,N-diisopropylethylamine (4.0 equiv). The crude was purified by flash chromatography (DCM 100% to DCM/MeOH 80:20) then further purified by preparative HPLC (30% to 70% ACN/H₂O) and freeze-dried to obtain Example 209 (69 mg, 64%) as a white solid.

¹H-NMR (DMSO-d₆, 300 MHz) δ: 1.53-1.62 (m, 1H, CH₂); 1.67-1.80 (m, 12H, 1.5*CH₂, C(CH₃)₃); 2.26 (s, 3H, CH₃); 3.49-3.61 (m, 1H, CH₂); 3.66-3.79 (m, 1H, CH₂); 3.81-3.90 (m, 2H, CH₂); 3.92-4.01 (m, 1H, CH₂); 4.28-4.38 (m, 1H, CH₂); 7.44 (d, 1H, J 8.4 Hz, Ar); 7.59-7.67 (m, 2H, Ar); 7.82 (dd, 1H, J 11.0, 1.8 Hz, Ar); 8.20 (s, 1H, Ar); 8.29 (s, 1H, NH), 8.43 (d, 1H, J 8.4 Hz, Ar).

M/Z (M[³⁵Cl]+H)⁺: 498.3

### Compound 537: tert-butyl 4-(4-(methoxycarbonyl)-3,5-dimethylphenyl)-2,2-dimethylpiperazine-1-carboxylate

Compound 537 was prepared according to general procedure (XXI) starting from methyl 4-bromo-2,6-dimethylbenzoate (206 mg) and tert-butyl 2,2-dimethylpiperazine-1-carboxylate (200 mg, 1.1 Eq). The crude was purified by flash chromatography (Merck 60®, CyHex 100% to CyHex/EtOAc 50:50) to obtain Compound 537 as a white solid (205 mg, 64%).

M/Z (M+H)⁺: 377.3

### Compound 538: methyl 4-(3,3-dimethylpiperazin-1-yl)-2,6-dimethylbenzoate hydrochloride

Compound 538 was prepared according to general procedure (XVIIIc) starting from Compound 537 (205 mg). The reaction mixture was concentrated under reduced pressure and triturated in Et₂O (15 mL) to obtain Compound 538 (166 mg, 97%) as a white solid.

M/Z (M+H)⁺: 277.3

### Compound 539: methyl 4-(4-(1-(tert-butyl)-3-(4-chloro-3-fluorophenyl)-1H-pyrrolo[2,3-b]pyridine-6-carbonyl)-3,3-dimethylpiperazin-1-yl)-2,6-dimethylbenzoate

Compound 539 was prepared according to general procedure (Xa) starting from Compound 78 (60 mg) and Compound 538 (60 mg, 1.1 equiv), and using triethylamine (4.0 equiv). The crude was purified by flash chromatography (CyHex 100% to CyHex/EtOAc 70:30) to obtain Compound 539 (90 mg, 86%) as a yellow solid.

M/Z (M[³⁵Cl]+H)⁺: 605.5

### Example 210: 4-(4-(1-(tert-butyl)-3-(4-chloro-3-fluorophenyl)-1H-pyrrolo[2,3-b]pyridine-6-carbonyl)-3,3-dimethylpiperazin-1-yl)-2,6-dimethylbenzoic acid

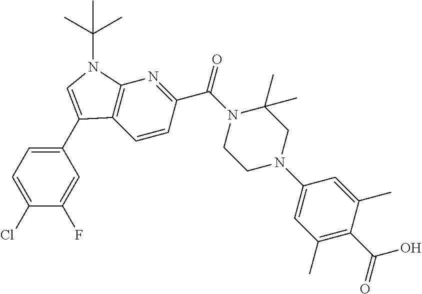 [SMILES: Cc1cc(N2CCN(C(=O)c3ccc4c(-c5ccc(Cl)c(F)c5)cn(C(C)(C)C)c4n3)C(C)(C)C2)cc(C)c1C(=O)O]

Example 210 was prepared according to general procedure (XIb) starting from Compound 539 (87 mg). The crude was purified by flash chromatography (Interchim® 20 μm, DCM 100% to DCM/MeOH 90:10), then triturated in MeOH and was purified by flash chromatography (DCM 100% to DCM/MeOH 93:7) and freeze-dried from a EtOH/water mixture to obtain Example 210 (10 mg, 12%) as a white solid.

¹H-NMR (DMSO-d₆, 300 MHz) δ: 1.56 (s, 6H, C(CH₃)₂); 1.82 (s, 9H, C(CH₃)₃); 2.26 (s, 6H, C(CH₃)₂); 3.40 (t, J 5.2 Hz, 2H, N—CH₂); 3.57 (s, 2H, N—CH₂); 3.83 (t, J 5.2 Hz, 2H, N—CH₂); 6.42 (s, 2H, 2 Ar); 7.41 (d, J 8.3 Hz, 1H, Ar); 7.60-7.69 (m, 2H, 2 Ar); 7.82 (dd, J 11.1 Hz, 1.7 Hz, 1H, Ar); 8.21 (s, 1H, Ar); 8.43 (d, J 8.3 Hz, 1H, Ar). 1 proton not observed.

M/Z (M[³⁵Cl]+H)⁺: 591.4.

### Compound 540: (S)-(tetrahydrofuran-2-yl)methyl methanesulfonate

Compound 540 was obtained (207 mg, n.d.) as a clear oil according to general procedure (XXIII) from (S)-(tetrahydrofuran-2-yl)methanol (100 mg) and methanesulfonyl chloride (135 mg, 1.2 equiv).

¹H-NMR (DMSO-d₆, 300 MHz) δ: 1.53-1.64 (m, 1H, CHₐH_{b}—CH₂—O); 1.79-2.03 (m, 3H, CHₐH_{b}—CH₂—O+CHₐH_{b}—CH—O); 3.18 (s, 3H, CH₃); 3.66-3.77 (m, 2H, CH₂—O); 4.07-4.21 (m, 3H, CH-0+CH₂—O—SO₂).

### Compound 541: (S)-6-chloro-3-(4-chloro-3-fluorophenyl)-1-((tetrahydrofuran-2-yl)methyl)-1H-pyrrolo[2,3-b]pyridine

Compound 541 was prepared according to general procedure (XIII) in NMP starting from Compound 9 (100 mg) and Compound 540 (109 mg). The reaction was stirred at 70° C. for 17 hours. The crude was purified by flash chromatography (CyHex 100% to CyHex/EtOAc 70:30) to obtain Compound 541 (100 mg, 77%) as a yellow oil.

M/Z (M[³⁵Cl]₂+H)⁺: 365.1.

### Compound 542: methyl (S)-6-(4-(3-(4-chloro-3-fluorophenyl)-1-((tetrahydrofuran-2-yl)methyl)-1H-pyrrolo[2,3-b]pyridine-6-carbonyl)-3,3-dimethylpiperazin-1-yl)-2,4-dimethylnicotinate

Compound 542 was prepared according to general procedure (XXX) starting from Compound 541 (100 mg) and Compound 95 (172 mg, 2.0 equiv). The crude was purified by flash chromatography (CyHex 100% to CyHex/EtOAc 50:50) to obtain Compound 542 (61 mg, 35%) as a yellow solid.

M/Z (M[³⁵Cl]+H)⁺: 634.5.

### Example 211: (S)-6-(4-(3-(4-chloro-3-fluorophenyl)-1-((tetrahydrofuran-2-yl)methyl)-1H-pyrrolo[2,3-b]pyridine-6-carbonyl)-3,3-dimethylpiperazin-1-yl)-2,4-dimethylnicotinic acid

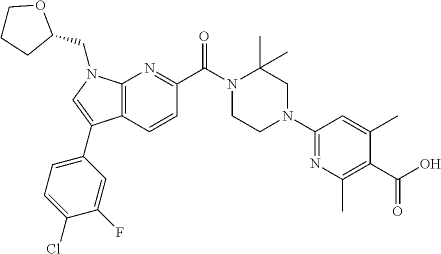 [SMILES: Cc1cc(N2CCN(C(=O)c3ccc4c(-c5ccc(Cl)c(F)c5)cn(C[C@@H]5CCCO5)c4n3)C(C)(C)C2)nc(C)c1C(=O)O]

Example 211 was prepared according to general procedure (XIb) starting from Compound 542 (61 mg). The crude was purified by preparative HPLC (30% to 70% ACN/H₂O), then freeze-dried with water to obtain Example 211 (38 mg, 63%) as a white solid.

¹H-NMR (DMSO-d₆, 300 MHz) δ: 1.54 (s, 6H, 2 CH₃); 1.63-1.71 (m, 1H, CH—(CHₐ—CH_{b}) of THF); 1.78-1.88 (m, 2H, CH₂ of THF); 1.94-2.05 (m, 1H, CH—(CHₐ—CH_{b}) of THF) of THF); 2.28 (s, 3H, CH₃—Ar); 2.40 (s, 3H, CH₃—Ar); 3.55-3.58 (m, 2H, CH₂); 3.62-3.69 (m, 1H, O—(CHₐ—CH_{b}) of THF); 3.81-3.85 (m, 3H, O—(CHₐ—CH_{b}) of THF+CH₂); 3.92 (m, 2H, CH₂); 4.24-4.32 (m, 1H, CH—O); 4.32-4.44 (m, 2H, CH₂—Ar); 6.37 (s, 1H, Ar); 7.42 (d, J 8.1 Hz, 1H, Ar); 7.60-7.68 (m, 2H, Ar); 7.75-7.78 (m, 1H, Ar); 8.24 (s, 1H, Ar); 8.47 (d, J 8.1 Hz, 1H, Ar); 12.73 (bs, 1H, COOH).

M/Z (M[³⁵Cl]+H)⁺: 620.4

### Compound 543: (R)-(tetrahydrofuran-2-yl)methyl methanesulfonate

Compound 543: was obtained (179 mg, n.d.) as a clear oil according to general procedure (XXIII) from (R)-(tetrahydrofuran-2-yl)methanol (100 mg) and methanesulfonyl chloride (135 mg, 1.2 equiv).

¹H-NMR (DMSO-d₆, 300 MHz) δ: 1.52-1.65 (m, 1H, CHₐH_{b}—CH₂—O); 1.79-2.02 (m, 3H, CHₐH_{b}—CH₂—O+CHₐH_{b}—CH—O); 3.18 (s, 3H, CH₃); 3.66-3.77 (m, 2H, CH₂—O); 4.07-4.21 (m, 3H, CH-0+CH₂—O—SO₂).

### Compound 544: (R)-6-chloro-3-(4-chloro-3-fluorophenyl)-1-((tetrahydrofuran-2-yl)methyl)-1H-pyrrolo[2,3-b]pyridine

Compound 544 was prepared according to general procedure (XIII) in NMP starting from Compound 9 (100 mg) and Compound 543 (109 mg). The reaction was stirred at 70° C. for 17 hours. The crude was purified by flash chromatography (CyHex 100% to CyHex/EtOAc 70:30) to obtain Compound 544 (53 mg, 41%) as a yellow oil.

M/Z (M[³⁵Cl]₂+H)⁺: 365.1.

### Compound 545: methyl (R)-6-(4-(3-(4-chloro-3-fluorophenyl)-1-((tetrahydrofuran-2-yl)methyl)-1H-pyrrolo[2,3-b]pyridine-6-carbonyl)-3,3-dimethylpiperazin-1-yl)-2,4-dimethylnicotinate

Compound 545 was prepared according to general procedure (XXX) starting from Compound 544 (53 mg) and Compound 95 (91 mg, 2.0 equiv). The crude was purified by flash chromatography (CyHex 100% to CyHex/EtOAc 30:70) to obtain Compound 545 (40 mg, 43%) as a yellow solid.

M/Z (M[³⁵Cl]+H)⁺: 634.4.

### Example 212: (R)-6-(4-(3-(4-chloro-3-fluorophenyl)-1-((tetrahydrofuran-2-yl)methyl)-1H-pyrrolo[2,3-b]pyridine-6-carbonyl)-3,3-dimethylpiperazin-1-yl)-2,4-dimethylnicotinic acid

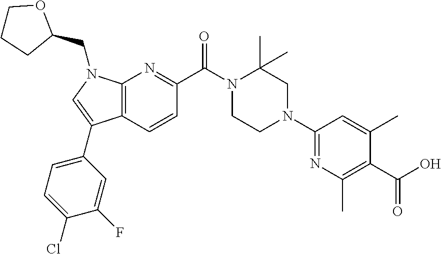 [SMILES: Cc1cc(N2CCN(C(=O)c3ccc4c(-c5ccc(Cl)c(F)c5)cn(C[C@H]5CCCO5)c4n3)C(C)(C)C2)nc(C)c1C(=O)O]

Example 212 was prepared according to general procedure (XIb) starting from Compound 545 (40 mg). The crude was purified by preparative HPLC (30% to 70% ACN/H₂O), then freeze-dried with water to obtain Example 212 (18 mg, 45%) as a white solid.

¹H-NMR (DMSO-d₆, 300 MHz) δ: 1.54 (s, 6H, 2 CH₃); 1.62-1.71 (m, 1H, CH—(CHₐ—CH_{b}) of THF); 1.78-1.87 (m, 2H, CH₂ of THF); 1.94-2.02 (m, 1H, CH—(CHₐ—CH_{b}) of THF); 2.27 (s, 3H, CH₃—Ar); 2.39 (s, 3H, CH₃—Ar); 3.55-3.58 (m, 2H, CH₂); 3.62-3.68 (m, 1H, O—(CHₐ—CH_{b}) of THF); 3.77-3.84 (m, 3H, O—(CHₐCH_{b}) of THF+CH₂); 3.92 (m, 2H, CH₂); 4.24-4.30 (m, 1H, CH—O); 4.34-4.42 (m, 2H, CH₂—Ar); 6.39 (s, 1H, Ar); 7.41 (d, J 8.1 Hz, 1H, Ar); 7.59-7.67 (m, 2H, Ar); 7.75-7.78 (m, 1H, Ar); 8.24 (s, 1H, Ar); 8.47 (d, J 8.1 Hz, 1H, Ar); COOH not observed.

M/Z (M[³⁵Cl]+H)⁺: 620.4.

### Compound 546: 2-(chloromethyl)-5-methylpyridine

To a solution of (5-methylpyridin-2-yl)methanol (100 mg, 1.0 equiv) in DCM (4.0 mL) was added thionyl chloride (4.0 mL). The reaction was stirred at 25° C. for 5 hours. The reaction mixture was concentrated to dryness to obtain Compound 546 (145 mg) as a clear oil.

M/Z (M[³⁵Cl]+H)⁺: 141.9.

### Compound 547: 6-chloro-3-(4-chloro-3-fluorophenyl)-1-((5-methylpyridin-2-yl)methyl)-1H-pyrrolo[2,3-b]pyridine

Compound 547 was prepared according to general procedure (XIII) in DMF from Compound 9 (150 mg) and Compound 546 (113 mg, 1.5 equiv). The reaction mixture was heated at 70° C. for 17 hours. The crude was purified by flash chromatography (CyHex 100% to CyHex/EtOAc 30:70) to obtain Compound 547 (58 mg, 28%) as a yellow oil.

M/Z (M[³⁵Cl]₂+H)⁺: 386.2.

### Compound 548: methyl 6-(4-(3-(4-chloro-3-fluorophenyl)-1-((5-methylpyridin-2-yl)methyl)-1H-pyrrolo[2,3-b]pyridine-6-carbonyl)-3,3-dimethylpiperazin-1-yl)-2,4-dimethylnicotinate

Compound 548 was prepared according to general procedure (XXX) starting from Compound 547 (58 mg) and Compound 95 (94 mg, 2.0 equiv). The crude was purified by flash chromatography (DCM 100% to DCM/MeOH 98:02) to obtain Compound 548 (34 mg, 35%) as a yellow solid.

M/Z (M[³⁵Cl]+H)⁺: 655.4.

### Example 213: 6-(4-(3-(4-chloro-3-fluorophenyl)-1-((5-methylpyridin-2-yl)methyl)-1H-pyrrolo[2,3-b]pyridine-6-carbonyl)-3,3-dimethylpiperazin-1-yl)-2,4-dimethylnicotinic acid

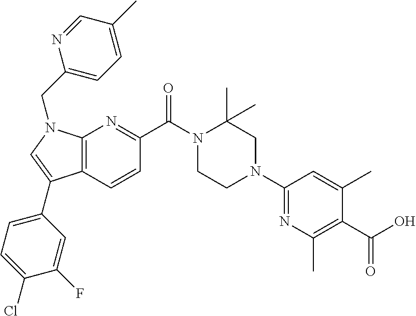 [SMILES: Cc1ccc(Cn2cc(-c3ccc(Cl)c(F)c3)c3ccc(C(=O)N4CCN(c5cc(C)c(C(=O)O)c(C)n5)CC4(C)C)nc32)nc1]

Example 213 was prepared according to general procedure (XIb) starting from Compound 548 (34 mg). The crude was purified by preparative HPLC (30% to 70% ACN/H₂O) and freeze-dried from water to obtain Example 213 (12 mg, 37%) as a white solid.

¹H-NMR (DMSO-d₆, 300 MHz) δ: 1.51 (s, 6H, 2 CH₃); 2.27 (s, 3H, CH₃—Ar); 2.37 (s, 3H, CH₃—Ar); 2.41 (s, 3H, CH₃—Ar); 3.64-3.67 (m, 2H, N—CH₂); 3.89 (m, 2H, N—CH₂); 5.55 (s, 2H, CH₂—Ar); 6.29 (s, 1H, Ar); 7.23 (d, J 8.2 Hz, 1H, Ar); 7.41 (d, J 8.2 Hz, 1H, Ar); 7.59 (dd, J 8.2, 2.0 Hz, 1H, Ar); 7.63-7.67 (m, 2H, Ar); 7.75-7.79 (m, 1H, Ar); 8.39 (s, 1H, Ar); 8.48 (d, J 8.2 Hz, 1H, Ar); 8.53 (d, J 2.0 Hz, 1H, Ar); 12.69 (bs, 1H, COOH); 2 protons not observed.

M/Z (M[³⁵Cl]+H)⁺: 641.4.

### Example 214: (1-(tert-butyl)-3-(4-chloro-3-fluorophenyl)-1H-pyrrolo[2,3-b]pyridin-6-yl)(1,1-dioxidothiomorpholino)methanone

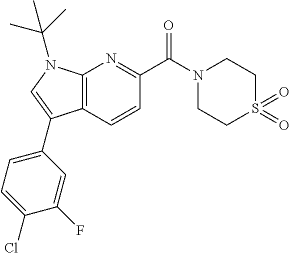 [SMILES: CC(C)(C)n1cc(-c2ccc(Cl)c(F)c2)c2ccc(C(=O)N3CCS(=O)(=O)CC3)nc21]

To a mixture of Compound 78 (60 mg, 1.0 equiv) in MeTFH (3 mL) was added (1-Cyano-2-ethoxy-2-oxoethylidenaminooxy)dimethylamino-morpholino-carbenium hexafluorophosphate (200 mg, 2.7 equiv), triethylamine (150 μL, 6.2 equiv) and the thiomorpholine 1,1-dioxide (35 mg, 1.5 equiv). The reaction mixture was stirred at 25° C. for 42 hours. The reaction mixture was concentrated under reduced pressure, the residue was purified by flash chromatography (CyHex 100% to EtOAc 100%), triturated in ACN and the solid was freeze dried from a EtOH/Water mixture from to obtain Example 214 (21 mg, 26%) as a white solid.

¹H-NMR (DMSO-d₆, 300 MHz) δ: 1.79 (s, 9H, C(CH₃)₃); 3.23-3.31 (m, 2H, CH₂); 3.34-3.40 (m, 2H, CH₂); 4.02-4.15 (m, 4H, 2*N—CH₂); 7.56 (d, J 8.3 Hz, 1H, Ar); 7.60-7.68 (m, 2H, 2 Ar); 7.83 (dd, J 11.1, 1.7 Hz, 1H, Ar); 8.24 (s, 1H, Ar); 8.47 (d, J 8.3 Hz, 1H, Ar).

M/Z (M[³⁵Cl]+H)⁺: 464.2.

### Compound 549: tert-butyl 4-(1-(tert-butyl)-3-(4-chloro-3-fluorophenyl)-1H-pyrrolo[2,3-b]pyridine-6-carbonyl)-3,3-dimethylpiperazine-1-carboxylate

Compound 549 was prepared according to general procedure (Xa) starting from Compound 78 (200 mg) and tert-butyl 3,3-dimethylpiperazine-1-carboxylate (136 mg, 1.1 equiv), and using N-ethyl-N-isopropylpropan-2-amine (3.0 equiv). Crude Compound 549 was obtained as a yellow solid (585 mg, n.d.).

M/Z (M[³⁵Cl]+H)⁺: 543.3

### Compound 550: (1-(tert-butyl)-3-(4-chloro-3-fluorophenyl)-1H-pyrrolo[2,3-b]pyridin-6-yl)(2,2-dimethylpiperazin-1-yl)methanone hydrochloride

Compound 550 was prepared according to general procedure (XVIIIb) starting from Compound 549 (585 mg). The reaction mixture was concentrated under reduced pressure and triturated in Et₂O (30 mL) to obtain Compound 550 (213 mg, 77%) as a yellow solid.

M/Z (M[³⁵Cl]+H)⁺: 443.2

### Compound 551: methyl 8-(4-(1-(tert-butyl)-3-(4-chloro-3-fluorophenyl)-1H-pyrrolo[2,3-b]pyridine-6-carbonyl)-3,3-dimethylpiperazin-1-yl)-8-oxooctanoate

Compound 551 was prepared according to general procedure (Xa) starting from Compound 550 (213 mg) and 8-methoxy-8-oxooctanoic acid (92 mg, 1.1 equiv), and using N-ethyl-N-isopropylpropan-2-amine (4.0 equiv). The crude was purified by flash chromatography (CyHex 100% to CyHex/EtOAc 50:50) to obtain Compound 551 (177 mg, 65%) as a yellow oil.

M/Z (M[³⁵Cl]+H)⁺: 613.5

### Example 215: 8-(4-(1-(tert-butyl)-3-(4-chloro-3-fluorophenyl)-1H-pyrrolo[2,3-b]pyridine-6-carbonyl)-3,3-dimethylpiperazin-1-yl)-8-oxooctanoic acid

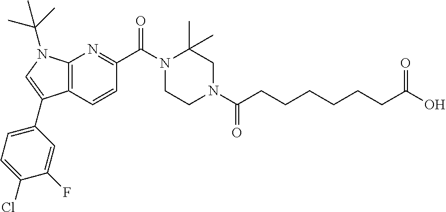 [SMILES: CC1(C)CN(C(=O)CCCCCCC(=O)O)CCN1C(=O)c1ccc2c(-c3ccc(Cl)c(F)c3)cn(C(C)(C)C)c2n1]

To a solution of Compound 551 (177 mg, 1.0 equiv) in THF (0.1 M) was added lithium hydroxide 1M aq. (1.0 equiv).

The reaction was stirred at 25° C. for 16 hours. The reaction mixture was acidified with HCl (1M aq., 40 mL) and extracted with EtOAc (2*40 mL). The organic layer was dried over magnesium sulfate and concentrated to dryness to afford Example 215 (166 mg, 96%) as a white solid.

¹H-NMR (DMSO-d₆, 300 MHz) δ: 1.25-1.31 (m, 4H, 2 CH₂); 1.45-1.51 (m, 7H, 2 CH₂+CH₃); 1.55 (s, 3H, CH₃); 1.78- 1.81 (m, 9H, tBu); 2.16-2.28 (m, 3H, CH₂+CHₐH_{b}); 2.31-2.37 (m, 1H, CHₐH_{b}); 3.42-3.46 (m, 1H, CHₐH_{b}); 3.62-3.65 (m, 3H, CHₐH_{b}+CH₂); 3.69-3.77 (m, 2H, CH₂); 7.39 (dd, J 8.4, 7.5 Hz, 1H, Ar); 7.59-7.68 (m, 2H, Ar); 7.79-7.84 (m, 1H, Ar); 8.21 (d, J 3.0 Hz, 1H, Ar); 8.42 (dd, J 8.4, 2.5 Hz, 2H, Ar); 11.96 (bs, 1H, COOH).

M/Z (M[³⁵Cl]+H)⁺: 599.3.

Mp: 73-83° C.

### Compound 552: 4-(tert-butyl)-2-(4-chlorophenyl)-1H-imidazole

To a solution of 4-chlorobenzimidamide hydrochloride (1.12 g, 1.05 equiv) in ACN (28 mL) was added potassium carbonate (2.32 g, 3.0 equiv) and 1-bromopinacolone (1.00 g, 1.0 equiv). The reaction was heated at 80° C. for 17 hours. The reaction mixture was hydrolyzed with water (100 mL) then extracted with EtOAc (2*100 mL). The combined organic layers were washed with brine (100 mL), dried over magnesium sulfate then concentrated to dryness. The crude was purified by flash chromatography (CyHex 100% to CyHex/EtOAc 90:10) to 552 (1.10 g, 84%) as a white solid.

M/Z (M[³⁵Cl]+H)⁺: 235.1

### Compound 553: 4-(tert-butyl)-2-(4-chlorophenyl)-5-nitro-1H-imidazole

To a solution of 552 (1.06 g, 1.0 equiv) in H₂SO₄ (22 mL) at 0° C. was added a solution of potassium nitrate (479 mg, 1.05 equiv) in H₂SO₄ (22 mL). The reaction was stirred at 0° C. for 30 min. The reaction mixture was hydrolyzed at 0° C. with water (100 mL) then extracted with EtOAc (100 mL). The organic layer was washed with brine (100 mL), dried over magnesium sulfate then concentrated to dryness to obtain the crude 553 (1.27 g, n.d) as a yellow solid.

M/Z (M[³⁵Cl]+H)⁺: 280.1

### Compound 554: methyl (Z/E)-3-(4-(tert-butyl)-2-(4-chlorophenyl)-5-nitro-1H-imidazol-1-yl)acrylate

To a solution of 553 (1.26 g, 1.0 equiv) in ACN (20 mL) was added methyl propiolate (603 μL, 1.5 equiv) and potassium carbonate (1.87 g, 3 equiv). The reaction was heated at 80° C. for 20 hours. Methyl propiolate (603 μL, 1.5 equiv) was added then the mixture was heated at 80° C. for 24 hour. The reaction mixture was hydrolyzed with NH4Cl sat. (100 mL) then extracted with EtOAc (100 mL). The organic layer was washed with brine (100 mL), dried over magnesium sulfate then concentrated to dryness. The crude was purified by flash chromatography (CyHex 100% to CyHex/EtOAc 90:10) to obtain Compound 554 (1.07 g, 65% over 2 steps) as a yellow solid.

M/Z (M[³⁵Cl]+H)⁺: 364.2

### Compound 555: 8-(tert-butyl)-6-(4-chlorophenyl)imidazo[1,5-a]pyrimidin-2-ol

To a solution of methyl 554 (1.07 g, 1.0 equiv) in AcOH (29 mL) was added iron (1.64 g, 10 equiv). The reaction was heated at 120° C. for 5 hours. The mixture was filtered through a pad of Celite washed with EtOAc (150 mL). The organic layer was washed with water (150 mL), NaOH 1N (150 mL), brine (150 mL), dried over magnesium sulfate then concentrated to dryness. The crude was purified by flash chromatography (CyHex 100% to CyHex/EtOAc 50:50) to obtain Compound 555 (430 mg, 48%) as a yellow solid.

M/Z (M[³⁵Cl]+H)⁺: 302.1

### Compound 556: 8-(tert-butyl)-2-chloro-6-(4-chlorophenyl)imidazo[1,5-a]pyrimidine

A suspension of 555 (185 mg, 1.0 equiv) in POCl₃ (2.87 mL, 50 equiv) was heated at 120° C. for 36 hours. The reaction mixture was concentrated to dryness then the brown oil was dissolved in POCl₃ (2.87 mL, 50 equiv). The reaction mixture was heated at 120° C. for 24 hours. The reaction mixture was quenched at 0° C. with NaOH 1N (200 mL) then extracted with EtOAc (200 mL). The organic layer was washed with brine (200 mL), dried over magnesium sulfate then concentrated to dryness. The crude was purified by flash chromatography (CyHex 100% to CyHex/EtOAc 90:10) to obtain Compound 556 (113 mg, 58%) as a yellow solid.

M/Z (M[³⁵Cl]₂+H)⁺: 320.0

### Example 216: ethyl 6-(4-(8-(tert-butyl)-6-(4-chlorophenyl)imidazo[1,5-a]pyrimidine-2-carbonyl)-3,3-dimethylpiperazin-1-yl)nicotinate

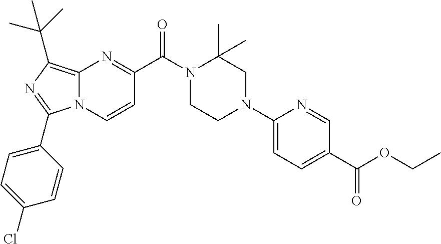 [SMILES: CCOC(=O)c1ccc(N2CCN(C(=O)c3ccn4c(-c5ccc(Cl)cc5)nc(C(C)(C)C)c4n3)C(C)(C)C2)nc1]

Example 216 was prepared according to general procedure (XXX) starting from Compound 556 (110 mg) and Compound 332 (154 mg, 1.5 equiv). The crude was purified by flash chromatography (Interchim® 20 μm, CyHex 100% to CyHex/EtOAc 70:30) to obtain Example 216 (100 mg, 51%) as a beige solid.

¹H-NMR (DMSO-d₆, 300 MHz) δ: 1.29 (t, J 7.1 Hz, 3H, O—CH₂—CH₃); 1.45-1.56 (m, 15H, 5*CH₃); 3.60-3.70 (m, 2H, N—CH₂); 3.98-4.10 (m, 4H, 2*N—CH₂); 4.20-4.30 (q, J 7.1 Hz, 2H, O—CH₂—CH₃); 6.67 (d, J 8.9 Hz, 1H, Ar); 6.90 (d, J 7.5 Hz, 1H, Ar); 7.62 (d, J 8.6 Hz, 2H, Ar); 7.89 (d, J 8.6 Hz, 2H, Ar); 7.99 (dd, J 8.9, 2.4 Hz, 1H, Ar); 8.67 (d, J 2.4 Hz, 1H, Ar); 8.82 (d, J 7.6 Hz, 1H, Ar).

M/Z (M[³⁵Cl]+H)⁺: 575.4

### Example 217: 6-(4-(8-(tert-butyl)-6-(4-chlorophenyl)imidazo[1,5-a]pyrimidine-2-carbonyl)-3,3-dimethylpiperazin-1-yl)nicotinic acid

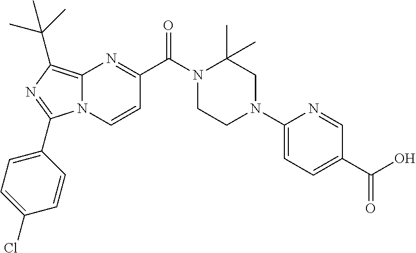 [SMILES: CC(C)(C)c1nc(-c2ccc(Cl)cc2)n2ccc(C(=O)N3CCN(c4ccc(C(=O)O)cn4)CC3(C)C)nc12]

To a solution of Example 216 (90 mg, 1.0 equiv) in THF (1 mL) and MeOH (1 mL) was added lithium hydroxide in water (0.23 mL, 1 molar, 1.5 equiv). The reaction was heated at 60° C. for 2 hours. The reaction mixture was diluted with water (20 mL). The aqueous layer was washed with Et₂O (20 mL) then acidified with HCl 1N until pH=4. The aqueous layer was extracted with DCM (2*20 mL). The combined organic layers were dried over magnesium sulfate then concentrated to dryness to obtain Example 217 (40 mg, 47%) as an orange solid.

¹H-NMR (DMSO-d₆, 300 MHz) δ: 1.53 (s, 15H, 5*CH₃); 3.58-3.68 (m, 2H, N—CH₂); 3.98-4.10 (m, 4H, 2*N—CH₂); 6.66 (d, J 8.9 Hz, 1H, Ar); 6.90 (d, J 7.5 Hz, 1H, Ar); 7.62 (d, J 8.6 Hz, 2H, Ar); 7.90 (d, J 8.6 Hz, 2H, Ar); 7.98 (dd, J 8.9, 2.4 Hz, 1H, Ar); 8.65 (d, J 2.4 Hz, 1H, Ar); 8.82 (d, J 7.6 Hz, 1H, Ar); 12.48 (bs, 1H, COOH).

M/Z (M[³⁵Cl]+H)⁺: 547.3.

### Compound 557: 4-bromo-N-methoxy-N-methylpicolinamide

At 0° C., To a suspension of 4-bromopicolinic acid (2.0 g, 1 equiv) in DCM (40 mL) were added HOBt (1.82 g, 1.2 equiv), 3-(((ethylimino)methylene)amino)-N,N-dimethylpropan-1-amine hydrochloride (2.28 g, 1.2 equiv), N,O-dimethylhydroxylamine hydrochloride (1.16 g, 1.2 equiv) and N-ethyl-N-isopropylpropan-2-amine (5.2 mL, 3.0 equiv). The reaction was stirred at 25° C. for 18 hours then concentrated under reduced pressure. The residue was diluted with EtOAc (50 mL), washed with NaHCO₃ sat., the aqueous phase was then extracted twice with EtOAc (2*50 mL), the organic extracts were dried over magnesium sulfate then concentrated to dryness. The crude was purified by flash chromatography (CyHex 100% to CyHex/EtOAc 50:50). The residue was triturated with Et₂O, the filtrate was evaporated to obtain Compound 557 (1.23 g, 51%) as a beige solid.

M/Z (M [⁸¹Br]+H)⁺: 247.0

### Compound 558: 1-(4-bromopyridin-2-yl)-3-methylbutan-1-one

At −78° C., to solution of Compound 557 (888 mg, 1.0 equiv) in THF (25 mL) was added dropwise isobutylmagnesium bromide (4.53 mL, 2.0 molar, 2.5 equiv). The reaction was stirred at −78° C. for 1 hour. The reaction mixture was quenched with NH₄Cl sat. (10 mL) then extracted with EtOAc (2*30 mL). The organic layer was dried over magnesium sulfate then concentrated to dryness. The crude was purified by flash chromatography (CyHex 100% to CyHex/Et₂O 50:50) to obtain Compound 558 (220 mg, 25%) as a clear oil.

M/Z (M[¹Br]+H)⁺: 244.0

### Compound 559: 7-bromo-3-(4-chlorophenyl)-1-isobutylimidazo[1,5-a]pyridine

To a solution of Compound 558 (220 mg, 1.0 equiv) in MeOH (3 mL) was added (4-chlorophenyl)methanamine (222 μL, 2 equiv) then titanium(IV) isopropoxide (1.10 mL, 4 equiv). The reaction was stirred at 25° C. for 6 hours. The reaction mixture was filtered on celite pad, and the filtrate was concentrated under reduced pressure. The crude material was taken up in DCE (6 mL), sodium acetate (112 mg, 1.5 equiv) and I₂ (277 mg, 1.2 equiv) were added. The reaction was heated at 80° C. for 18 hours then quenched with saturated Na₂S₂O₃ (20 mL) and diluted with EtOAc (20 mL), the mixture was filtered on celite pad. The filtrate was extracted with EtOAc (3*50 mL). Combined organic layers was dried over magnesium sulfate then concentrated to dryness. The crude was purified by flash chromatography (Interchim® 20 μm, CyHex 100% to CyHex/EtOAc 85:15) to obtain Compound 559 (68 mg) as a yellow solid.

M/Z (M[³⁵Cl] [⁸¹Br]+H) 365.1

### Example 218: ethyl 6-(4-(3-(4-chlorophenyl)-1-isobutylimidazo[1,5-a]pyridine-7-carbonyl)-3,3-dimethylpiperazin-1-yl)nicotinate

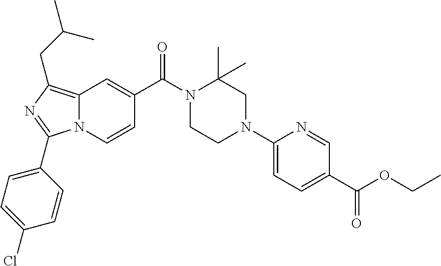 [SMILES: CCOC(=O)c1ccc(N2CCN(C(=O)c3ccn4c(-c5ccc(Cl)cc5)nc(CC(C)C)c4c3)C(C)(C)C2)nc1]

Example 218 was prepared according to general procedure (XXX) starting from Compound 559 (68 mg) and Compound 332 (84 mg, 1.5 equiv). The crude was purified by flash chromatography (Interchim® 20 μm, CyHex 100% to CyHex/EtOAc 70:30) to obtain Example 218 (50 mg, 47% over 2 steps) as a beige solid.

¹H-NMR (DMSO-d₆, 300 MHz) δ: 0.93 (d, J 6.7 Hz, 6H, 2*N—CH₂—CH—CH₃); 1.29 (t, J 7.1 Hz, 3H, O—CH₂—CH₃); 1.52 (s, 6H, 2*CH₃); 1.99-2.10 (m, 1H, N—CH₂—CH—CH₃); 2.78 (d, J 6.9 Hz, 2H, N—CH₂—CH—CH₃); 3.55-3.65 (m, 2H, N—CH₂); 3.83-3.90 (m, 2H, N—CH₂); 3.95-4.02 (m, 2H, N—CH₂); 4.26 (q, J 7.1 Hz, 2H, O—CH₂—CH₃); 6.63-6.75 (m, 2H, Ar); 7.62 (d, J 8.6 Hz, 2H, Ar); 7.75-7.79 (m, 1H, Ar); 7.88 (d, J 8.6 Hz, 2H, Ar); 7.97 (dd, J 8.9, 2.4 Hz, 1H, Ar); 8.40 (d, J 7.3 Hz, 1H, Ar); 9.64 (d, J 2.4 Hz, 1H, Ar).

M/Z (M[³⁵Cl]+H)⁺: 574.3.

### Example 219: 6-(4-(3-(4-chlorophenyl)-1-isobutylimidazo[1,5-a]pyridine-7-carbonyl)-3,3-dimethylpiperazin-1-yl)nicotinic acid

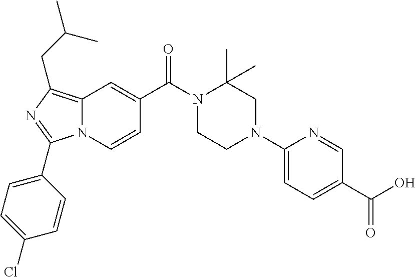 [SMILES: CC(C)Cc1nc(-c2ccc(Cl)cc2)n2ccc(C(=O)N3CCN(c4ccc(C(=O)O)cn4)CC3(C)C)cc12]

To a solution of Example 218 (45 mg, 1.0 equiv) in THF (0.5 mL) and MeOH (0.5 mL) was added lithium hydroxide in water (0.12 mL, 1 molar, 1.5 equiv). The reaction was heated at 60° C. for 2 hours. The reaction mixture was diluted with water (20 mL). The aqueous layer was washed with Et₂O (20 mL) then acidified with HCl 1N until pH=4. The aqueous layer was extracted with DCM (2*20 mL). The combined organic layers were dried over magnesium sulfate then concentrated to dryness to obtain Example 219 (18 mg, 42%) as a beige solid.

¹H-NMR (DMSO-d₆, 300 MHz) δ: 0.94 (d, J 6.7 Hz, 6H, 2*N—CH₂—CH—CH₃); 1.52 (s, 6H, 2*CH₃); 1.99-2.10 (m, 1H, N—CH₂—CH—CH₃); 2.78 (d, J 6.9 Hz, 2H, N—CH₂—CH—CH₃); 3.55-3.65 (m, 2H, N—CH₂); 3.83-3.90 (m, 2H, N—CH₂); 3.95-4.02 (m, 2H, N—CH₂); 6.63-6.75 (m, 2H, Ar); 7.62 (d, J 8.6 Hz, 2H, Ar); 7.75-7.79 (m, 1H, Ar); 7.88 (d, J 8.6 Hz, 2H, Ar); 7.97 (dd, J 8.9, 2.4 Hz, 1H, Ar); 8.40 (d, J 7.3 Hz, 1H, Ar); 9.64 (d, J 2.4 Hz, 1H, Ar); 12.44 (bs, 1H, COOH).

M/Z (M[³⁵Cl]+H)⁺: 546.3.

### Compound 560: 1-(2,6-dichloropyridin-3-yl)-1-(3-fluoro-4-(trifluoromethyl)phenyl)ethan-1-ol

Compound 560 was prepared according to general procedure (I) starting from 2,6-dichloropyridine (3.00 g) and 1-(3-fluoro-4-(trifluoromethyl)phenyl)ethan-1-one (5.00 g). The crude was purified by flash chromatography (CyHex 100% to CyHex/EtOAc 50:50) to obtain Compound 560 (4.17 g, 50%) as a yellow oil. Compound 560 is contaminated with 1-(2,6-dichloropyridin-4-yl)-1-(3-fluoro-4-(trifluoromethyl)phenyl)ethan-1-ol (around 20%).

M/Z (M[³⁵Cl]₂+H)⁺: 354.0

### Compound 561: 2,6-dichloro-3-(1-(3-fluoro-4-(trifluoromethyl)phenyl)vinyl)pyridine

Compound 561 was obtained (3.70 g, n.d.) as a brown oil according to general procedure (II) starting from Compound 560 (4.17 g). Compound 561 is contaminated with 2,6-dichloro-4-(1-(3-fluoro-4-(trifluoromethyl)phenyl)vinyl)pyridine (around 20%).

M/Z (M[³⁵Cl₂]+H)⁺: 336.1

### Compound 562: 2,6-dichloro-3-(2-(3-fluoro-4-(trifluoromethyl)phenyl)oxiran-2-yl)pyridine

Compound 562 was prepared according to general procedure (III) starting from Compound 561 (3.70 g). Compound 562 (3.60 g, n.d.) was obtained after the optional purification step and purification by flash chromatography (CyHex 100% to CyHex/EtOAc 75:25), as a colourless oil. Compound 562 is contaminated with 2,6-dichloro-3-(2-(4-chloro-3-fluorophenyl)oxiran-2-yl)pyridine (around 20%).

M/Z (M[³⁵Cl₂]+H)⁺: 352.0

### Compound 563: 1-(tert-butyl)-6-chloro-3-(3-fluoro-4-(trifluoromethyl)phenyl)-1H-pyrrolo[2,3-b]pyridine

Compound 563 was prepared according to general procedure (IV) starting from Compound 562 (3.60 g) and 2-methylpropan-2-amine (2.7 mL, 2.5 equiv) in DMA. The crude was purified by flash chromatography (CyHex 100% to CyHex/EtOAc 97:03) to obtain Compound 563 (1.74 g, 45%) as a white solid.

M/Z (M[³⁵Cl]+H)⁺: 371.1

### Compound 564: methyl 6-(4-(1-(tert-butyl)-3-(3-fluoro-4-(trifluoromethyl)phenyl)-1H-pyrrolo[2,3-b]pyridine-6-carbonyl)-3,3-dimethylpiperazin-1-yl)-2,4-dimethylnicotinate

Compound 564 was prepared according to general procedure (XXX) starting from Compound 563 (150 mg) and Compound 95 (254 mg, 2.0 equiv). The crude was purified by flash chromatography (CyHex 100% to CyHex/EtOAc 80:20) to obtain Compound 564 (203 mg, 78%) as a yellow oil.

M/Z (M+H)⁺: 640.5.

### Example 220: 6-(4-(1-(tert-butyl)-3-(3-fluoro-4-(trifluoromethyl)phenyl)-1H-pyrrolo[2,3-b]pyridine-6-carbonyl)-3,3-dimethylpiperazin-1-yl)-2,4-dimethylnicotinic acid

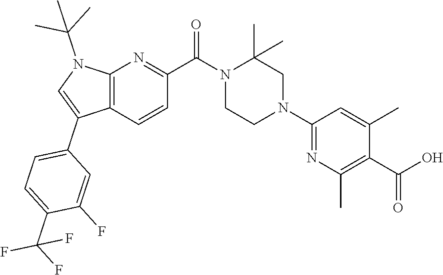 [SMILES: Cc1cc(N2CCN(C(=O)c3ccc4c(-c5ccc(C(F)(F)F)c(F)c5)cn(C(C)(C)C)c4n3)C(C)(C)C2)nc(C)c1C(=O)O]

Example 220 was prepared according to general procedure (XIb) starting from Compound 564 (203 mg). The crude was purified by flash chromatography (DCM 100% to DCM/MeOH 95:5), triturated in Et₂O, and freeze-dried from an Ethanol/water (1:5) mixture to obtain Example 220 (120 mg, 60%) as a white solid.

¹H-NMR (DMSO-d₆, 300 MHz) δ: 1.55 (s, 6H, 2 CH₃ of piperazine); 1.83 (s, 9H, tBu); 2.27 (s, 3H, CH₃—Ar); 2.38 (s, 3H, CH₃—Ar); 3.52-3.58 (m, 2H, CH₂); 3.83-3.87 (m, 2H, CH₂); 3.93 (s, 2H, CH₂); 6.32 (s, 1H, Ar); 7.44 (d, J 8.3 Hz, 1H, Ar); 7.77-7.85 (m, 2H, Ar); 7.93 (d, J 12.6 Hz, 1H, Ar); 8.35 (s, 1H, Ar); 8.51 (d, J 8.3 Hz, 1H, Ar). COOH not observed.

M/Z (M+H)⁺: 626.4.

### Compound 565: 3-(tert-butyl)-1-(4-chloro-3-fluorophenyl)-1H-pyrrolo[3,2-b]pyridine-5-carboxylic acid

In a bridged two-vial under inert atmosphere, in chamber 1, to a solution of Compound 475 (180 mg) in 1,4-Dioxane (1.1 mL) was added triethylamine (161 μL, 4 equiv). In chamber 2, molybdenum hexacarbonyl (114 mg, 1.5 equiv) was suspended in 1,4-Dioxane (1.1 mL). Both chambers were sparged with argon for 10 min. XantPhos Pd G4 (27 mg, 0.1 equiv) was added to chamber 1 and DBU (196 μL, 4.5 equiv) was added to chamber 2. The reaction was heated at 110° C. for 20 h. The mixture of chamber 1 was filtered through a pad of Celite which was washed with EtOAc (50 mL). The organic layer was washed with aqueous HCl (1M, 50 mL), brine (50 mL), dried over magnesium sulfate then concentrated to dryness. The crude residue was purified by flash chromatography (CyHex 100% to CyHex/EtOAc 50:50) to afford Compound 565 (60 mg, 43%) as a yellow solid.

M/Z (M[³⁵Cl]+H)⁺: 347.0.

### Example 221: 4-(3-(tert-butyl)-1-(4-chloro-3-fluorophenyl)-1H-pyrrolo[3,2-b]pyridine-5-carbonyl)-3,3-dimethylpiperazin-2-one

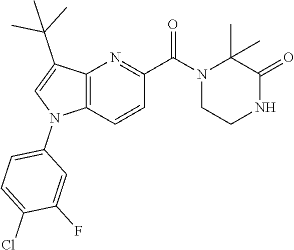 [SMILES: CC(C)(C)c1cn(-c2ccc(Cl)c(F)c2)c2ccc(C(=O)N3CCNC(=O)C3(C)C)nc12]

Example 221 was prepared according to general procedure (Xa) starting from Compound 565 (30 mg) and 3,3-dimethylpiperazin-2-one (12.2 mg, 1.1 equiv), and using N-ethyl-N-isopropylpropan-2-amine (3.0 equiv). The crude was purified by preparative HPLC (10% to 50% ACN/H₂O) and freeze-dried from water to obtain Example 221 (10 mg, 26%) as a white solid.

¹H-NMR (DMSO-d₆, 300 MHz) δ: 1.49 (s, 9H, C(CH₃)₃); 1.72 (s, 6H, (CH₃)₂); 3.35-3.37 (m, 2H, N—CH₂); 3.61-3.64 (m, 2H, N—CH₂); 7.50 (d, J 8.8 Hz, 1H, Ar); 7.56 (ddd, J 8.8, 2.4, 1.0 Hz, 1H, Ar); 7.79 (t, J 8.8 Hz, 1H, Ar); 7.83-7.87 (m, 2H, Ar); 8.10 (d, J 8.8 Hz, 1H, Ar); 8.13-8.14 (m, 1H, NH).

M/Z (M[³⁵Cl]+H)⁺: 457.2.

### Compound 566: methyl 2-(1-(3-(tert-butyl)-1-(4-chloro-3-fluorophenyl)-1H-pyrrolo[3,2-b]pyridine-5-carbonyl)piperidin-4-yl)acetate

Compound 566 was prepared according to general procedure (Xa) starting from Compound 565 (30 mg) and methyl 2-(piperidin-4-yl)acetate (15 mg, 1.1 equiv), and using N-ethyl-N-isopropylpropan-2-amine (3.0 equiv). The crude was purified by flash chromatography (CyHex 100% to CyHex/EtOAc 50:50) to obtain Compound 566 (13 mg, 26%) as a clear oil.

M/Z (M[³⁵Cl]+H)⁺: 486.3.

### Example 222: 2-(1-(3-(tert-butyl)-1-(4-chloro-3-fluorophenyl)-1H-pyrrolo[3,2-b]pyridine-5-carbonyl)piperidin-4-yl)acetic acid

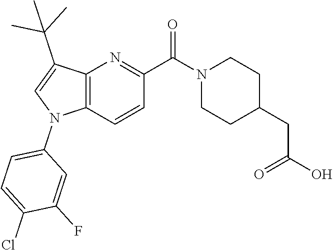 [SMILES: CC(C)(C)c1cn(-c2ccc(Cl)c(F)c2)c2ccc(C(=O)N3CCC(CC(=O)O)CC3)nc12]

To a solution of compound 566 (12 mg, 1.0 equiv) in THF (1 mL) was added lithium hydroxide 1M aq. (37 μL, 1.5 equiv). The reaction was stirred at 25° C. for 17 hours. Further lithium hydroxide 1M aq. (37 μL, 1.5 equiv) was added and the reaction was stirred at 60° C. for 4 days. Further lithium hydroxide 1M aq. (37 μL, 1.5 equiv) was added and the reaction was stirred at 60° C. for another 7 days. The reaction mixture was acidified with HCl (1M aq., 3 mL) and the precipitate was recovered by filtration and washed with water (2*3 mL) to afford Example 222 (7 mg, 61%) as a white solid.

¹H-NMR (DMSO-d₆, 300 MHz) δ: 1.18 (m, 2H, CH—CH₂); 1.50 (s, 9H, C(CH₃)₃); 1.63-1.68 (m, 1H, CH—CHₐH_{b}); 1.79-1.84 (m, 1H, CH—CHₐH_{b}); 1.93-2.05 (m, 1H, CH₂—CH—CH₂); 2.19-2.22 (m, 2H, CH₂); 2.81-2.89 (m, 1H, N—CHₐH_{b}); 3.05-3.13 (m, 1H, N—CHₐH_{b}); 4.10-4.15 (m, 1H, N—CHₐH_{b}); 4.50-4.54 (m, 1H, N—CHₐH_{b}); 7.45 (d, J 8.6 Hz, 1H, Ar); 7.56 (ddd, J 8.6, 2.4, 0.8 Hz, 1H, Ar); 7.78 (t, J 8.6 Hz, 1H, Ar); 7.80 (s, 1H, Ar); 7.85 (dd, J 10.6, 2.5 Hz, 1H, Ar); 8.09 (d, J 8.6 Hz, 1H, Ar); 12.14 (bs, 1H, COOH).

M/Z (M[³⁵Cl]+H)⁺: 472.2.

### Example 223: 4-(3-(benzo[d][1,3]dioxol-5-yl)-1-isobutyl-1H-pyrrolo[2,3-b]pyridine-6-carbonyl)-3,3-dimethylpiperazin-2-one

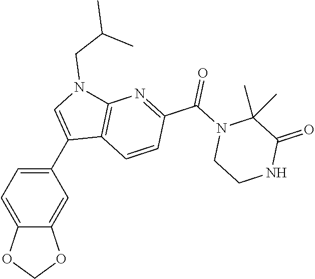 [SMILES: CC(C)Cn1cc(-c2ccc3c(c2)OCO3)c2ccc(C(=O)N3CCNC(=O)C3(C)C)nc21]

Example 223 was prepared according to general procedure (XVIIa) starting from Compound 99 (100 mg) and 2-(benzo[d][1,3]dioxol-5-yl)-4,4,5,5-tetramethyl-1,3,2-dioxaborolane (73 mg, 1.2 equiv). The crude was purified by flash chromatography (Interchim® 20 μm, CyHex/EtOAc 50:50 to EtOAc 100%) then recrystallized from hot ACN (5 mL) to obtain Example 223 (32 mg, 29%) as a white solid.

¹H-NMR (DMSO-d₆, 300 MHz) δ: 0.87 (d, J 6.6 Hz, 6H, CH(CH₃)₂); 1.72 (s, 6H, C(CH₃)₂); 2.14-2.30 (m, 1H, CH(CH₃)₂); 3.50-3.58 (m, 2H, N—CH₂); 4.09 (d, J 7.5 Hz, 2H, CH₂CH); 6.05 (s, 2H, O—CH₂—O); 7.00 (d, J 8.1 Hz, 1H, Ar); 7.19 (d, J 8.1 Hz, 1H, Ar); 7.26 (s, 1H, Ar); 7.41 (d, J 8.1 Hz, 1H, Ar); 8.02 (s, 1H, Ar); 8.11 (s, 1H, NH); 8.59 (d, J 8.1 Hz, 1H, Ar). 2 protons not observed.

M/Z (M+H)⁺: 449.2

### Example 224: 4-(3-(benzofuran-2-yl)-1-isobutyl-1H-pyrrolo[2,3-b]pyridine-6-carbonyl)-3,3-dimethylpiperazin-2-one

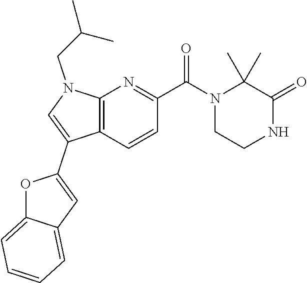 [SMILES: CC(C)Cn1cc(-c2cc3ccccc3o2)c2ccc(C(=O)N3CCNC(=O)C3(C)C)nc21]

Example 224 was prepared according to general procedure (XVIIa) starting from Compound 99 (100 mg) and benzofuran-2-ylboronic acid (72 mg, 1.8 equiv). The crude was purified by flash chromatography (Interchim® 20 μm, CyHex/EtOAc 50:50 to EtOAc 100%) then recrystallized from hot ACN (5 mL) and freeze dried from ethanol/water to obtain Example 224 (20 mg, 18%) as a white solid.

¹H-NMR (DMSO-d₆, 300 MHz) δ: 0.88 (d, J 6.7 Hz, 6H, CH(CH₃)₂); 1.73 (s, 6H, C(CH₃)₂); 2.17-2.27 (m, 1H, CH(CH₃)₂); 3.53-3.55 (m, 2H, N—CH₂); 4.17 (d, J 7.2 Hz, 2H, CH₂CH); 7.22-7.32 (m, 3H, Ar); 7.52 (d, J 8.2 Hz, 1H, Ar); 7.59-7.64 (m, 2H, Ar); 8.11 (s, 1H, NH); 8.35 (s, 1H, Ar); 8.59 (d, J 8.2 Hz, 1H, Ar). 2 protons not observed.

M/Z (M+H)⁺: 445.3

### Example 225: 4-(3-(5-fluoro-1H-indol-2-yl)-1-isobutyl-1H-pyrrolo[2,3-b]pyridine-6-carbonyl)-3,3-dimethylpiperazin-2-one

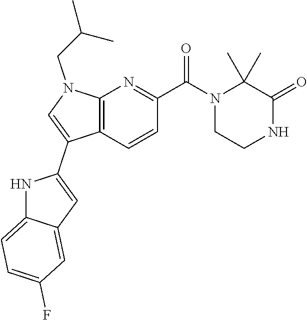 [SMILES: CC(C)Cn1cc(-c2cc3cc(F)ccc3[nH]2)c2ccc(C(=O)N3CCNC(=O)C3(C)C)nc21]

Example 225 was prepared according to general procedure (XVIIa) starting from Compound 99 (100 mg) and (1-(tert-butoxycarbonyl)-5-fluoro-1H-indol-2-yl)boronic acid (97 mg, 1.4 equiv). The crude was purified by flash chromatography (Interchim® 20 μm, CyHex/EtOAc 50:50 to EtOAc 100%) then taken up in DCM (4 mL) and treated with TFA (4 mL) for 16 hours. The reaction mixture was neutralized with NaHCO₃ (sat. aq., 50 mL) and extracted with DCM (2*10 mL). The organic layer was dried over magnesium sulfate then concentrated to dryness. The crude was recrystallized from hot ACN (5 mL) and let air dry to obtain Example 225 (40 mg, 35%) as a brown solid.

¹H-NMR (DMSO-d₆, 300 MHz) δ: 0.88 (d, J 6.7 Hz, 6H, CH(CH₃)₂); 1.73 (s, 6H, C(CH₃)₂); 2.16-2.22 (m, 1H, CH(CH₃)₂); 3.53-3.57 (m, 2H, N—CH₂); 4.15 (d, J 7.2 Hz, 2H, CH₂CH); 6.84-6.93 (m, 2H, Ar); 7.26 (dd, J 10.1 2.5 Hz, 1H, Ar); 7.36 (dd, J 8.8 4.7 Hz, 1H, Ar); 7.49 (d, J 8.2 Hz, 1H, Ar); 8.11 (s, 1H, NH); 8.18 (s, 1H, Ar); 8.53 (d, J 8.2 Hz, 1H, Ar); 11.5 (s, 1H, NH); 2 protons not observed.

M/Z (M+H)⁺: 462.5

### Example 226: 4-(1-isobutyl-3-(naphthalen-2-yl)-1H-pyrrolo[2,3-b]pyridine-6-carbonyl)-3,3-dimethylpiperazin-2-one

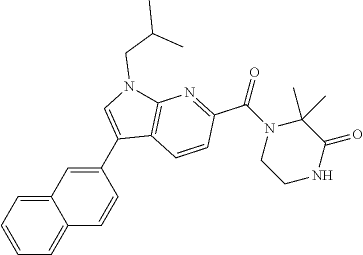 [SMILES: CC(C)Cn1cc(-c2ccc3ccccc3c2)c2ccc(C(=O)N3CCNC(=O)C3(C)C)nc21]

Example 226 was prepared according to general procedure (XVIIa) starting from Compound 99 (100 mg) and naphthalen-2-ylboronic acid (72 mg, 1.7 equiv). The crude was purified by flash chromatography (Interchim® 20 μm, CyHex/EtOAc 50:50 to EtOAc 100%) then further purified by preparative HPLC (Column B, 20% to 100% ACN/H₂O), then freeze-dried with water to obtain Example 226 (13 mg, 12%) as a white solid.

¹H-NMR (DMSO-d₆, 300 MHz) δ: 0.90 (d, J 6.7 Hz, 6H, CH(CH₃)₂); 1.73 (s, 6H, C(CH₃)₂); 2.20-2.32 (m, 1H, CH(CH₃)₂); 3.55-3.61 (m, 2H, N—CH₂); 4.15 (d, J 7.2 Hz, 2H, CH₂CH); 7.45-7.57 (m, 3H, Ar); 7.89-7.95 (m, 2H, Ar); 7.96-8.05 (m, 2H, Ar); 8.12 (s, 1H, NH); 8.27-8.31 (m, 2H, Ar); 8.61 (d, J 8.2 Hz, 1H, Ar). 2 protons not observed.

M/Z (M+H)⁺: 431.4

### Compound 567: tert-butyl methyl(2-(N-methylpalmitamido)ethyl)carbamate

To a solution of Palmitic acid (200 mg) in THF (7.8 mL), was added N-ethyl-N-isopropylpropan-2-amine (0.4 mL, 3 equiv) and ((1H-benzo[d][1,2,3]triazol-1-yl)oxy)tris(dimethylamino)phosphonium hexafluorophosphate (448 mg, 1.3 equiv). The reaction mixture was stirred at 25° C. for 5 minutes then tert-butyl methyl(2-(methylamino)ethyl)carbamate (147 mg, 1.1 equiv) was added and the mixture was stirred for 16 hours. The reaction mixture was hydrolysed with NH4Cl (sat. aq. 50 mL) and extracted with EtOAc (2*40 mL). The organic layer was washed with brine (50 mL), dried over magnesium sulfate, and concentrated to dryness to obtain Compound 567 (610 mg, n.d.) as a yellow oil.

M/Z (M+H)⁺: 427.4.

### Compound 568: N-methyl-N-(2-(methylamino)ethyl)palmitamide

To a solution of Compound 567 (780 μmol) in DCM (3.9 mL) was added HCl (2M in Et20, 3.9 mL, 10 equiv), the reaction mixture was stirred at 25° C. for 21 hours. The reaction mixture was diluted with HCl (1M aq., 50 mL), washed with DCM (40 mL), then basified with aqueous NaOH 6M to pH>12 and extracted with DCM (2*75 mL). The organic layers were dried over magnesium sulfate and to dryness to obtain Compound 568 (308 mg, n.d.) as a yellow oil.

M/Z (M+H)⁺: 327.4.

### Compound 569: tert-butyl (13,16-dimethyl-12,17-dioxo-3,6,9-trioxa-13,16-diazadotriacontyl)carbamate

To a solution of 2,2-dimethyl-4-oxo-3,8,11,14-tetraoxa-5-azaheptadecan-17-oic acid (276 mg, 1.1 equiv) in THF (7.8 mL), was added N-ethyl-N-isopropylpropan-2-amine (0.4 mL, 3 equiv) and ((1H-benzo[d][1,2,3]triazol-1-yl)oxy)tris(dimethylamino)phosphonium hexafluorophosphate (448 mg, 1.3 equiv). The reaction mixture was stirred at 25° C. for 5 minutes then Compound 568 (780 μmol) was added and the mixture was stirred for 16 hours. The reaction mixture was hydrolysed with NH4Cl (sat. aq. 50 mL) and extracted with EtOAc (2*40 mL). The organic layer was washed with brine (50 mL), dried over magnesium sulfate, and concentrated to dryness to obtain Compound 569 (665 mg, n.d.) as a yellow oil.

M/Z (M+H)⁺: 630.6

### Compound 570: N-(1-amino-13-methyl-12-oxo-3,6,9-trioxa-13-azapentadecan-15-yl)-N-methylpalmitamide

To a solution of Compound 569 (780 μmol) in DCM (3.9 mL) was added HCl (2M in Et20, 3.9 mL, 10 equiv), the reaction mixture was stirred at 25° C. for 65 hours. The reaction mixture was basified with NaOH (1M, aq. 50 mL) and extracted with EtOAc (2*40 mL). The organic layers were dried over magnesium sulfate and to dryness to obtain Compound 570 (398 mg, n.d.) as a yellow oil.

M/Z (M+H)⁺: 530.6

### Compound 571: tert-butyl (13,16-dimethyl-12,17-dioxo-3,6,9-trioxa-13,16-diazadotriacontyl)carbamate

To a solution of 2,2-dimethyl-4-oxo-3,8,11,14-tetraoxa-5-azaheptadecan-17-oic acid (266 mg, 1.1 equiv) in THF (7.5 mL), was added N-ethyl-N-isopropylpropan-2-amine (0.4 mL, 3 equiv) and ((1H-benzo[d][1,2,3]triazol-1-yl)oxy)tris(dimethylamino)phosphonium hexafluorophosphate (432 mg, 1.3 equiv). The reaction mixture was stirred at 25° C. for 5 minutes then Compound 570 (780 μmol) was added and the mixture was stirred for 16 hours. The reaction mixture was hydrolysed with NH4Cl (sat. aq. 50 mL) and extracted with EtOAc (2*40 mL). The organic layer was washed with brine (50 mL), dried over magnesium sulfate, and concentrated to dryness to obtain Compound 571 (918 mg, n.d.) as a yellow oil.

M/Z (M-Boc+H)⁺: 733.7

### Compound 572: N-(1-amino-26-methyl-12,25-dioxo-3,6,9,16,19,22-hexaoxa-13,26-diazaoctacosan-28-yl)-N-methylpalmitamide

To a solution of Compound 571 (780 μmol) in DCM (3.9 mL) was added HCl (2M in Et20, 3.9 mL, 10 equiv), the reaction mixture was stirred at 25° C. for 20 hours. The reaction mixture was basified with NaOH (1M, aq. 50 mL) and extracted with EtOAc (2*40 mL). The organic layers were dried over magnesium sulfate and to dryness to obtain Compound 572 (502 mg, n.d.) as a yellow oil.

M/Z (M+H)⁺: 733.7

### Example 227: N-(37-(4-(1-(tert-butyl)-3-(4-chloro-3-fluorophenyl)-1H-pyrrolo[2,3-b]pyridine-6-carbonyl)-3,3-dimethylpiperazin-1-yl)-3-methyl-4,17,30,37-tetraoxo-7,10,13,20,23,26-hexaoxa-3,16,29-triazaheptatriacontyl)-N-methylpalmitamide

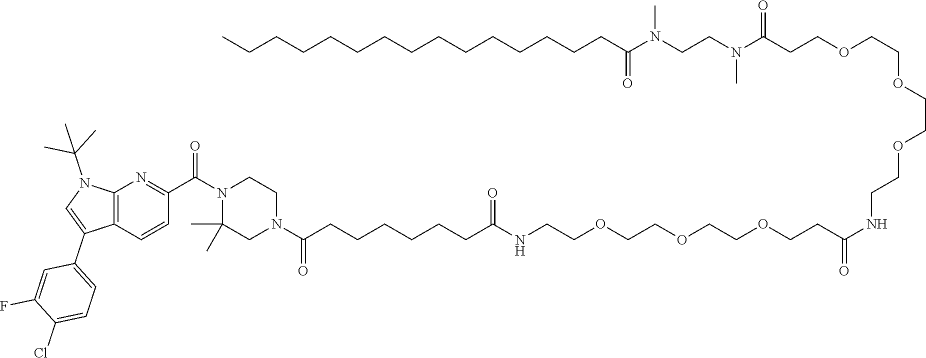 [SMILES: CCCCCCCCCCCCCCCC(=O)N(C)CCN(C)C(=O)CCOCCOCCOCCNC(=O)CCOCCOCCOCCNC(=O)CCCCCCC(=O)N1CCN(C(=O)c2ccc3c(-c4ccc(Cl)c(F)c4)cn(C(C)(C)C)c3n2)C(C)(C)C1]

To a solution of Example 215 (25 mg, 1.0 equiv) in THF (1 mL), was added N-ethyl-N-isopropylpropan-2-amine (22 μL, 3 equiv) and ((1H-benzo[d][1,2,3]triazol-1-yl)oxy)tris(dimethylamino)phosphonium hexafluorophosphate (28 mg, 1.5 equiv). The reaction mixture was stirred at 25° C. for 5 minutes then Compound 572 (40 mg, 1.3 equiv) was added and the mixture was stirred for 1 hour. The reaction mixture was hydrolysed with NH4Cl (sat. aq. 50 mL) and extracted with EtOAc (2*40 mL). The organic layer was washed with brine (50 mL), dried over magnesium sulfate, and concentrated to dryness. The crude was purified by preparative HPLC (Column B, 50% to 100% ACN/H₂O), then freeze-dried twice from a water/ACN mixture (1:1) to obtain Example 227 (38 mg, 69%) as a yellow oil.

¹H-NMR (CDCl₃, 300 MHz) δ: 0.82-0.90 (m, 5H, CH₃+CH₂); 1.35-1.41 (m, 2H, CH₂); 1.55-1.69 (m, 13H, 2 CH₃₊₃ CH₂+CHₐH_{b}); 1.83-1.85 (m, 12H, CHₐH_{b}+tBu+CH₂); 1.87-1.93 (m, 12H, 2N—CH₃₊₃ CH₂); 2.21-2.40 (m, 7H, 3 CH₂+CHₐH_{b}); 2.47-2.65 (m, 5H, CHₐH_{b}+2 CH₂); 2.95-2.97 (m, 2H, CH₂); 3.03-3.04 (m, 5H, 2 CH₂+CHₐH_{b}); 3.44-3.67 (m, 35H, CHₐH_{b}+17 CH₂); 3.73-3.81 (m, 6H, 3 CH₂); 3.85-3.94 (m, 2H, CH₂); 7.30-7.39 (m, 2H, Ar); 7.42-7.48 (m, 1H, Ar); 7.53 (dd, J 8.1, 4.7 Hz, 1H, Ar); 7.62 (d, J 2.3 Hz, 1H, Ar); 8.20 (dd, J 8.1, 2.3 Hz, 1H, Ar).

M/Z (M[³⁵Cl]+2H)²⁺/2: 657.7.

### Compound 573: methyl 3-iodo-1H-indazole-5-carboxylate

To a suspension of methyl 1H-indazole-5-carboxylate (1.0 g, 1.0 equiv) in Acetonitrile (5 mL) and DMF (5 mL) was added NIS (1.3 g, 1 equiv). The reaction was stirred at 25° C. for 24 hours. The reaction mixture was quenched with Na₂S₂O₃ (10% aqueous, 70 mL) then extracted with EtOAc (3×50 mL). The organic layer was washed with brine (50 mL), dried over magnesium sulfate then concentrated to dryness. The crude was purified by flash chromatography (CyHex 100% to EtOAc/CyHex 80:20) to obtain Compound 573 (1.2 g, 70%) as a yellow solid.

M/Z (M+H)⁺: 302.9.

### Compound 574: methyl 3-isobutyl-1H-indazole-5-carboxylate

To a solution of Compound 573 (600 mg, 1 equiv) in THF (10 mL) was added Bromo(isobutyl)zinc (0.5 M in THF, 11.9 mL, 3 equiv). The reaction mixture was sparged with argon for 10 minutes then Pd-PEPPSI-iPent (157 mg, 0.1 equiv) was added. The reaction mixture was heated at 75° C. for 5 hours. The mixture was filtered through a pad of Celite washed with EtOAc (4×10 mL). The organic layer was washed with NH4Cl (sat. aqueous, 50 mL), brine (50 mL), dried over magnesium sulfate then concentrated to dryness. The crude compound was purified by flash chromatography (CyHex 100% to CyHex/EtOAc 60:40) to obtain Compound 574 (343 mg, 74%) as a white solid.

M/Z (M+H)⁺: 233.0.

### Compound 575: methyl 1-(4-chloro-3-fluorophenyl)-3-isobutyl-1H-indazole-5-carboxylate

Compound 575 was prepared according to general procedure (XXVIII) starting from Compound 574 (150 mg) and (4-chloro-3-fluorophenyl)boronic acid (225 mg, 2 equiv). The crude residue was purified by flash chromatography (CyHex 100% to CyHex/EtOAc 70:30) to obtain Compound 575 (200 mg, 86%) as a white solid.

M/Z (M[³⁵Cl]+H)⁺: 361.1

### Compound 576: 1-(4-chloro-3-fluorophenyl)-3-isobutyl-1H-indazole-5-carboxylic acid

Compound 576 was obtained (155 mg, 81%) as a white solid according to general procedure (IXa) starting from Compound 575 (200 mg).

M/Z (M[³⁵Cl]+H)⁺: 347.2

### Example 228: 4-(1-(4-chloro-3-fluorophenyl)-3-isobutyl-1H-indazole-5-carbonyl)-3,3-dimethylpiperazin-2-one

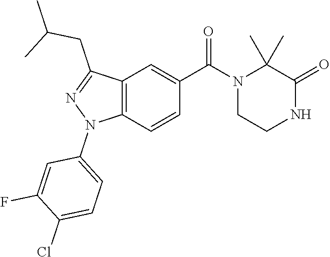 [SMILES: CC(C)Cc1nn(-c2ccc(Cl)c(F)c2)c2ccc(C(=O)N3CCNC(=O)C3(C)C)cc12]

Example 228 was prepared according to general procedure (Xa) starting from Compound 576 (65 mg) and 3,3-dimethylpiperazin-2-one (55 mg, 2.0 equiv), and using N-ethyl-N-isopropylpropan-2-amine (3.0 equiv). The crude was purified by flash chromatography (CyHex 100% to EtOAc 100%) to obtain Example 228 (40 mg, 40%) as a white solid.

¹H-NMR (DMSO-d₆, 300 MHz) δ: 0.97 (d, J 6.9 Hz, 6H, —CH—CH₂—(CH₃)₂); 1.72 (s, 6H, C(CH₃)₂); 2.06-2.19 (m, 1H, —CH—CH₂—(CH₃)₂); 2.91 (d, J 7.2 Hz, 2H, —CH—CH₂—(CH₃)₂); 3.25-3.29 (m, 2H, N—CH₂—CH₂—); 3.38-3.42 (m, 2H, N—CH₂—CH₂—); 7.55 (dd, J 8.7, 1.2 Hz, 1H, Ar); 7.69 (dd, J 8.7, 1.8 Hz, 1H, Ar); 7.76 (d, J 8.1 Hz, 1H, Ar); 7.80-7.87 (m, 1H, Ar); 7.93 (d, J 9.0 Hz, 1H, Ar); 7.99 (s, 1H, Ar); 8.10 (bs, 1H, NH).

M/Z (M[³⁵Cl]+H)+457.3.

Mp: 182-185° C.

### Biological Examples

In vitro human PAR-2 functional antagonist activity using calcium assays

Examples of the present invention were tested successively for their agonist and antagonist activities on human PAR-2 (hPAR-2) receptor expressed in HEK-293 T cells in an endogenous manner. Compounds exert agonist activity if, by themselves in absence of the 2-Furoyl-LIGRLO-NH₂ peptide (selective hPAR-2 agonist), they activate PAR-2; they exert antagonist activity if they decrease the action of the 2-Furoyl-LIGRLO-NH₂ peptide on the receptor. The assay used to determine compound activity is based on calcium measurement.

Cell Culture: HEK-293 T cells were maintained in Dulbecco's Modified Eagle's Medium supplemented with 10% Foetal Calf Serum, 1% Penicillin/Streptomycin at 37° C./5% CO₂. Flasks for tests (F75) are seeded with 6e+06 cells 24 h before the experiment at 37° C./5% CO₂.

Calcium assay: Receptor activity was detected by changes in intracellular calcium measured using the fluorescent Ca²⁺ sensitive dye, Fluo4AM (Molecular Probes).

The day of the assay, medium was replaced by assay buffer (HBSS 1× (Gibco 14175-053), Hepes 20 mM, MgSO₄-7H₂O 1 mM, Na₂CO₃ 3.3 mM, CaCl₂-2H₂O 1.3 mM, 0.5% BSA, Probenecid 2.5 mM, 0.1 mg/mL Pluronic Acid) and complemented with 1 μM Fluo4AM and cells were incubated during 1:30 h at 37° C.

Cells were then detached, resuspended in assay buffer and seeded in 384 wells, transparent-flat bottom black walled plates (2e+04 cells per well). Plates were incubated at +22° C. for 20 min. Compounds stock solutions were prepared in 100% DMSO and dilutions in test buffer were carried out in polypropylene plates with a digital dispenser (D300e, Tecan). Compounds were added to the cells and intracellular Ca²⁺ measurements were collected at the same time on FLIPR Tetra® (Molecular Devices) with specific filters (Exc: 470-495 nm/Em: 515-575 nm).

Agonist and antagonist activities of compounds were consecutively evaluated on the same cell plate. Agonist activity was first measured after 10 min incubation with the compound alone. Then, cells were stimulated by an EC₈₀ of the 2-Furoyl-LIGRLO-NH₂ peptide and fluorescence was recorded for an additional 5 min. EC₈₀ of the 2-Furoyl-LIGRLO-NH₂ peptide was the concentration giving 80% of the maximal 2-Furoyl-LIGRLO-NH₂ response. Agonist activities are evaluated in comparison to basal signals evoked by assay buffer or maximal 2-Furoyl-LIGRLO-NH₂ response. None of the compounds tested were found to exhibit PAR-2 agonist activity. Antagonist activities are evaluated in comparison to basal signals evoked by assay buffer or EC₈₀ of 2-Furoyl-LIGRLO-NH₂ alone.

For IC₅₀ determination, a concentration-response test was performed using 20 concentrations (ranging over 4.5 logs) of each compound. Dose-response curves were fitted using a sigmoidal dose-response 4 parameters (variable slope) analysis in XLfit Excel addon (IDBS) and IC₅₀ of antagonist activity was calculated. Concentration-response experiments were performed in duplicate, in two independent experiments. IC₅₀ values are categorized as following: A: IC₅₀<20 nM; B: 20 nM<IC₅₀<10 μM; C: IC₅₀>10 μM
(A)—IC₅₀<20 nM: Examples 1, 2, 3, 5, 6, 7, 8, 26, 28, 29, 35, 38, 59, 95, 96, 97, 98, 99, 104, 107, 121, 124, 126, 129, 131, 134, 136, 137, 138, 139, 140, 141, 142, 143, 144, 151, 152, 160, 161, 165, 166, 171, 172, 176, 177, 181, 183, 184, 185, 190, 191, 192, 193, 205, 207, 210, 211, 212, 215, 220, 221.
(B)—IC₅₀ between 20 nM and 10 μM: Examples 4, 13, 14, 15, 16, 17, 18, 19, 20, 21, 22, 23, 24, 27, 30, 31, 34, 39, 40, 41, 42, 43, 48, 49, 50, 51, 52, 53, 54, 55, 56, 57, 61, 62, 65, 67, 71, 72, 73, 75, 76, 77, 79, 80, 82, 83, 84, 85, 87, 100, 101, 102, 103, 105, 106, 108, 109, 110, 111, 112, 113, 114, 115, 116, 117, 118, 119, 120, 122, 123, 125, 127, 130, 132, 135, 146, 147, 148, 149, 155, 156, 157, 158, 159, 162, 163, 164, 167, 168, 169, 170, 173, 174, 175, 178, 179, 180, 182, 187, 188, 195, 197, 198, 199, 200, 201, 202, 203, 204, 214, 217, 219, 223, 224, 225, 226, 228.
(C)—IC₅₀>10 μM: Examples 9, 10, 11, 12, 32, 33, 36, 37, 44, 45, 46, 47, 58, 60, 63, 64, 66, 69, 70, 74, 78, 81, 88, 89, 90, 91, 92, 93, 94, 128, 133, 145, 153, 154, 186, 189, 194, 206, 208, 209, 216, 218, 222.

It has thus been demonstrated that the compounds of formula (I), including in particular the above-described examples, are potent antagonists of PAR-2.

1. A compound of the following formula (I) 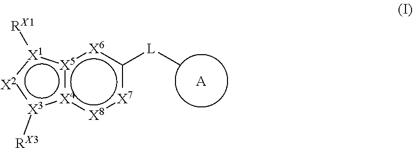
wherein:
the bicyclic ring system containing the ring atoms X¹ to X⁸ is aromatic;
one, two, three or four of the ring atoms X¹ to X⁸ are nitrogen atoms, and all remaining ring atoms are carbon atoms;
any among the ring atoms X², X⁶, X⁷ and X⁸ that is a carbon atom is optionally substituted with a group R^{X};
each R^{X} is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-NH—OH, —(C₀₋₃ alkylene)-N(C₁₋₅ alkyl)-OH, —(C₀₋₃ alkylene)-NH—O(C₁₋₅ alkyl), —(C₀₋₃ alkylene)-N(C₁₋₅ alkyl)-O(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-NH—COO(C₁₋₅ alkyl), —(C₀₋₃ alkylene)-N(C₁₋₅ alkyl)-COO(C₁₋₅ alkyl), —(C₀₋₃ alkylene)-O—CO—NH(C₁₋₅ alkyl), —(C₀₋₃ alkylene)-O—CO—N(C₁₋₅ alkyl)(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-SO—(C₁₋₅ alkyl), —(C₀₋₃ alkylene)-SO₂—(C₁₋₅ alkyl), —(C₀₋₃ alkylene)-carbocyclyl, —(C₀₋₃ alkylene)-heterocyclyl, and -L¹-R^{L1}, wherein the carbocyclyl group in said —(C₀₋₃ alkylene)-carbocyclyl and the heterocyclyl group in said —(C₀₋₃ alkylene)-heterocyclyl are each optionally substituted with one or more groups R^{Cyc};
R^{X1} is selected from C₁₋₅ alkyl, C₂₋₅ alkenyl, C₂₋₅ alkynyl, —(C₀₋₅ alkylene)-carbocyclyl, and —(C₀₋₅ alkylene)-heterocyclyl, wherein said alkyl, said alkenyl, said alkynyl, the alkylene group in said —(C₀₋₅ alkylene)-carbocyclyl, and the alkylene group in said —(C₀₋₅ alkylene)-heterocyclyl are each optionally substituted with one or more groups R^{X11}, wherein one or more —CH₂— units comprised in said alkyl, said alkenyl, said alkynyl, in the alkylene group in said —(C₀₋₅ alkylene)-carbocyclyl, or in the alkylene group in said —(C₀₋₅ alkylene)-heterocyclyl are each optionally replaced by a group independently selected from —O—, —NH—, —N(C₁₋₅ alkyl)-, —CO—, —S—, —SO—, and —SO₂—, and further wherein the carbocyclyl group in said —(C₀₋₅ alkylene)-carbocyclyl and the heterocyclyl group in said —(C₀₋₅ alkylene)-heterocyclyl are each optionally substituted with one or more groups R^{Cyc};
each R^{X11} is independently selected from —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O—(C₁₋₅ haloalkyl), —CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO—(C₁₋₅ alkyl), —SO₂—(C₁₋₅ alkyl), carbocyclyl, heterocyclyl, and -L¹-R^{L1}, wherein said carbocyclyl and said heterocyclyl are each optionally substituted with one or more groups R^{Cyc};
R^{X3} is -L^{X3}-carbocyclyl or -L^{X3}-heterocyclyl, wherein the carbocyclyl in said -L^{X3}-carbocyclyl or the heterocyclyl in said -L^{X3}-heterocyclyl is optionally substituted with one or more groups R^{X31};
L^{X3} is independently selected from a bond, —C(R^{LX3})(R^{LX3})—, —O—, —S—, —SO—, —SO₂—, —CO—, and —N(R^{LX3})—, wherein each R^{LX3} is independently hydrogen or C₁₋₅ alkyl, and further wherein two groups R^{LX3} which are attached to the same carbon atom may also be mutually joined to form, together with the carbon atom that they are attached to, a cycloalkyl or a heterocycloalkyl;
each R^{X31} is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-NH—OH, —(C₀₋₃ alkylene)-N(C₁₋₅ alkyl)-OH, —(C₀₋₃ alkylene)-NH—O(C₁₋₅ alkyl), —(C₀₋₃ alkylene)-N(C₁₋₅ alkyl)-O(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-NH—COO(C₁₋₅ alkyl), —(C₀₋₃ alkylene)-N(C₁₋₅ alkyl)-COO(C₁₋₅ alkyl), —(C₀₋₃ alkylene)-O—CO—NH(C₁₋₅ alkyl), —(C₀₋₃ alkylene)-O—CO—N(C₁₋₅ alkyl)(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-SO—(C₁₋₅ alkyl), —(C₀₋₃ alkylene)-SO₂—(C₁₋₅ alkyl), —(C₀₋₃ alkylene)-carbocyclyl, —(C₀₋₃ alkylene)-heterocyclyl, and -L¹-R^{L1}, wherein the carbocyclyl group in said —(C₀₋₃ alkylene)-carbocyclyl and the heterocyclyl group in said —(C₀₋₃ alkylene)-heterocyclyl are each optionally substituted with one or more groups R^{Cyc};
L is selected from —CO—, —SO— and —SO₂—;
ring A is a 5 to 14 membered heterocyclyl which is attached via a ring nitrogen atom to group L, wherein said heterocyclyl is optionally substituted with one or more groups R^{A};
each R^{A} is independently selected from C₁₋₅ alkyl, C₂₋₅ alkenyl, C₂₋₅ alkynyl, —(CO—3 alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-NH—OH, —(C₀₋₃ alkylene)-N(C₁₋₅ alkyl)-OH, —(C₀₋₃ alkylene)-NH—O(C₁₋₅ alkyl), —(C₀₋₃ alkylene)-N(C₁₋₅ alkyl)-O(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-NH—COO(C₁₋₅ alkyl), —(C₀₋₃ alkylene)-N(C₁₋₅ alkyl)-COO(C₁₋₅ alkyl), —(C₀₋₃ alkylene)-O—CO—NH(C₁₋₅ alkyl), —(C₀₋₃ alkylene)-O—CO—N(C₁₋₅ alkyl)(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-SO—(C₁₋₅ alkyl), —(C₀₋₃ alkylene)-SO₂—(C₁₋₅ alkyl), —(C₀₋₃ alkylene)-carbocyclyl, —(C₀₋₃ alkylene)-heterocyclyl, and -L¹-R^{L1}, wherein the carbocyclyl group in said —(C₀₋₃ alkylene)-carbocyclyl and the heterocyclyl group in said —(C₀₋₃ alkylene)-heterocyclyl are each optionally substituted with one or more groups R^{Cyc}; and further wherein any two groups R^{A}, which are attached to the same carbon ring atom of ring A, may also be mutually joined to form, together with the carbon ring atom that they are attached to, a cycloalkyl or a heterocycloalkyl, wherein said cycloalkyl or said heterocycloalkyl is optionally substituted with one or more groups R^{Cyc};
each R^{Cyc} is independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O—(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), —P(═O)(—OH)(—OH), —P(═O)(—OH)(—O—C₁₋₅ alkyl), —P(═O)(—O—C₁₋₅ alkyl)(-O—C₁₋₅ alkyl), —(C₀₋₃ alkylene)-cycloalkyl, —(C₀₋₃ alkylene)-heterocycloalkyl, and -L¹-R^{L1};
each L¹ is independently selected from a covalent bond, C₁₋₇ alkylene, C₂₋₇ alkenylene, and C₂₋₇ alkynylene, wherein said alkylene, said alkenylene and said alkynylene are each optionally substituted with one or more groups independently selected from halogen, C₁₋₅ haloalkyl, —O—(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl), and further wherein one or more —CH₂— units comprised in said alkylene, said alkenylene or said alkynylene are each optionally replaced by a group independently selected from —O—, —NH—, —N(C₁₋₅ alkyl)-, —CO—, —S—, —SO—, and —SO₂—; and
each R^{L1} is independently selected from —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O—(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), aryl, heteroaryl, cycloalkyl, and heterocycloalkyl, wherein said aryl, said heteroaryl, said cycloalkyl, and said heterocycloalkyl are each optionally substituted with one or more groups independently selected from C₁₋₅ alkyl, C₂₋₅ alkenyl, C₂₋₅ alkynyl, halogen, C₁₋₅ haloalkyl, —O—(C₁₋₅ haloalkyl), —CN, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO—(C₁₋₅ alkyl), and —SO₂—(C₁₋₅ alkyl);
or a pharmaceutically acceptable salt or solvate thereof;
wherein the following conditions apply:
if X¹, X² and X⁷ are nitrogen atoms, X³, X⁴, X⁵, X⁶ and X⁸ are carbon atoms, and L is —CO—, then R^{X3} is aryl, -L^{X3}-cycloalkyl, or monocyclic heteroaryl, wherein said aryl, the cycloalkyl in said -L^{X3}-cycloalkyl, or said heteroaryl is optionally substituted with one or more groups R^{X31};
if X², X³, X⁶ and X⁸ are nitrogen atoms, X¹, X⁴, X⁵ and X⁷ are carbon atoms, L is —CO—, and ring A is a monocyclic heterocyclyl which is attached via a ring nitrogen atom to group L and which is optionally substituted with one or more groups R^{A}, then the carbon atom in position X⁷ is not substituted with C₁₋₅ alkyl, —OH, —O(C₁₋₅ alkyl), —NH₂, —NH(C₁₋₅ alkyl), —N(C₁₋₅ alkyl)(C₁₋₅ alkyl), or phenyl;
if X¹ and X² are nitrogen atoms, X³, X⁴ and X⁵ are carbon atoms, L is —CO—, and R^{X1} is pyrimidin-2-yl which is optionally substituted with one or more groups R^{Cyc}, then R^{X3} is not cycloalkyl;
if X³ is a nitrogen atom, X¹, X⁴ and X⁵ are carbon atoms, and R^{X1} is phenyl, pyridin-2-yl or pyrimidin-2-yl wherein said phenyl, said pyridin-2-yl or said pyrimidin-2-yl is optionally substituted with one or more groups R^{Cyc}, then R^{X3} is not cyclopropyl;
if X³ and X⁸ are nitrogen atoms, X¹, X², X⁴, X⁵, X⁶ and X⁷ are carbon atoms, L is —CO—, and ring A is 2,5-dimethyl-4-(4-fluorobenzyl)-piperazin-1-yl, then R^{X3} is not 4-methoxybenzyl;
if X¹ is a nitrogen atom, X², X³, X⁴, X⁵, X⁶, X⁷ and X⁸ are carbon atoms, and L is —CO—, then ring A is not 4-(C₁₋₅ alkyl)-piperazin-1-yl or 4-(C₃₋₇ cycloalkyl)-piperazin-1-yl;
if X¹ is a nitrogen atom, X³, X⁴, X⁵, X⁶, X⁷ and X⁸ are carbon atoms, and L is —CO—, then R^{X3} is not a 9-membered bicyclic heteroaryl which is optionally substituted with one or more groups R^{X31};
if X¹ is a nitrogen atom, X², X³, X⁴, X⁵, X⁶, X⁷ and X⁸ are carbon atoms, L is —CO—, and R^{X1} is —CH₃ or —SO₂—CH₃, then R^{X3} is not —CH₂-heterocycloalkyl wherein the heterocycloalkyl in said —CH₂— heterocycloalkyl is optionally substituted with one or more groups R^{X31};
if X¹ is a nitrogen atom, X², X³, X⁴, X⁵, X⁶, X⁷ and X⁸ are carbon atoms, L is —CO—, ring A is morpholin-4-yl, and R^{X1} is 4-carboxythiazol-2-yl, then R^{X3} is not 4-sulfamoylbenzyl;
if X³ is a nitrogen atom, X¹, X², X⁴, X⁵, X⁶, X⁷ and X⁸ are carbon atoms, L is —CO—, ring A is morpholin-4-yl, and R^{X1} is 4-sulfamoylbenzyl, then R^{X3} is not 4-carboxythiazol-2-yl;
if X³ is a nitrogen atom, X¹, X², X⁴, X⁵, X⁶, X⁷ and X⁸ are carbon atoms, and L is —CO—, then L^{X3} is not —C(R^{LX3})(R^{LX3})—;
if X² and X³ are nitrogen atoms, X¹, X⁴, X⁵, X⁶, X⁷ and X⁸ are carbon atoms, and L is —SO₂—, then L^{X3} is not —CH₂—;
if X², X³ and X⁸ are nitrogen atoms, X¹, X⁴, X⁵, X⁶ and X⁷ are carbon atoms, L is —CO—, R^{X1} is methyl, and R^{X3} is pyridin-2-yl, then ring A is not piperidin-1-yl.

2. The compound of claim 1, wherein the bicyclic ring system 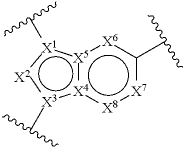
is selected from any one of the following groups: 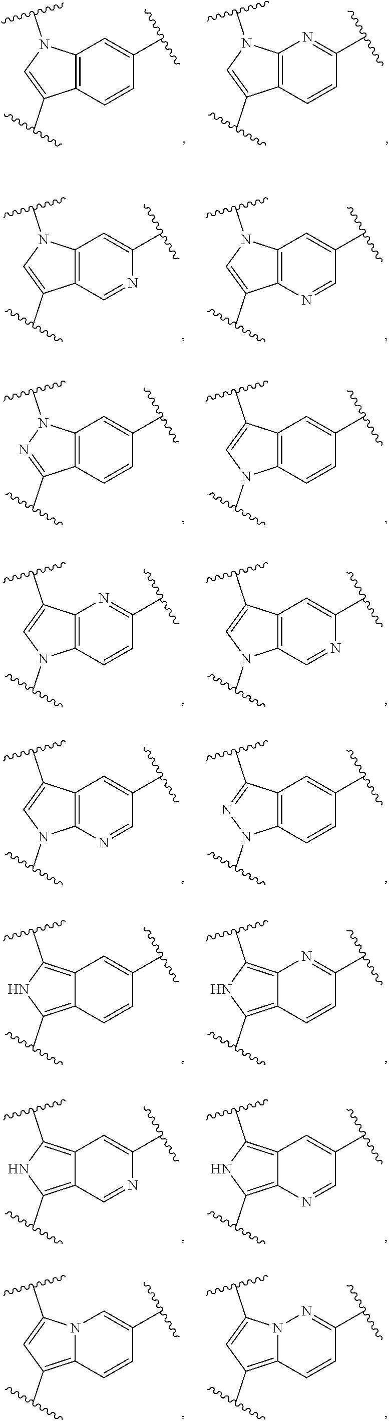 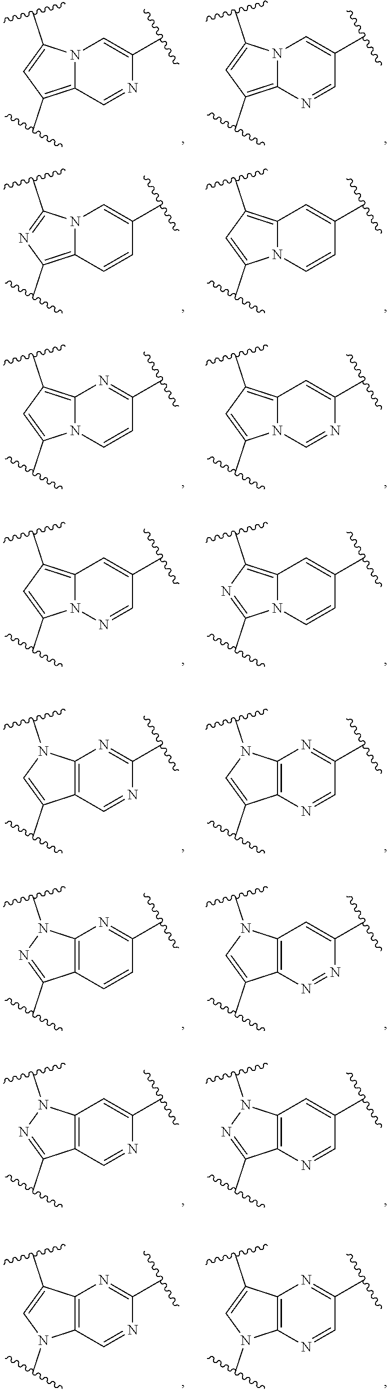 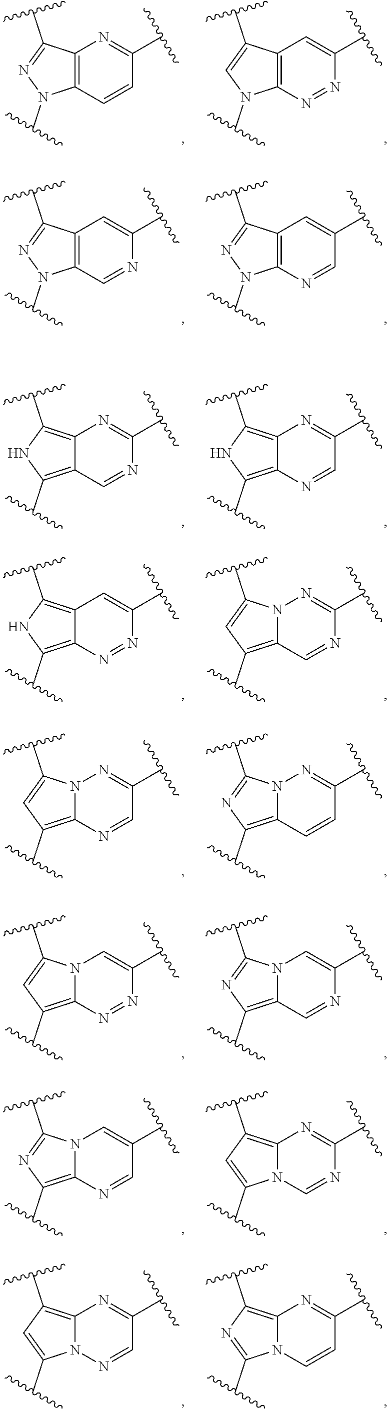 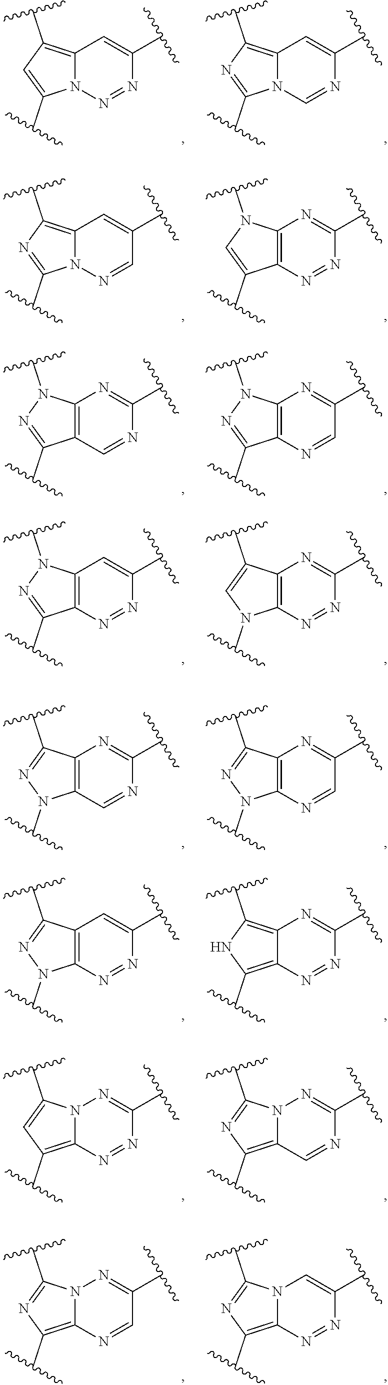 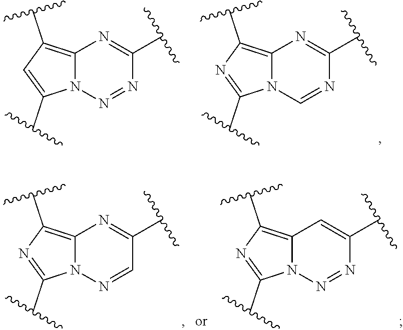 [SMILES: *c1nnn2c(*)cc(*)c2n1 ; *c1ncn2c(*)nc(*)c2n1 ; *c1cnn2c(*)nc(*)c2n1 ; *c1cc2c(*)nc(*)n2nn1]
wherein each of the above-depicted groups is optionally substituted with one or more groups R^{X}.

3. The compound of claim 1 or 2, wherein the bicyclic ring system 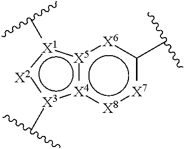
is selected from any one of the following groups: 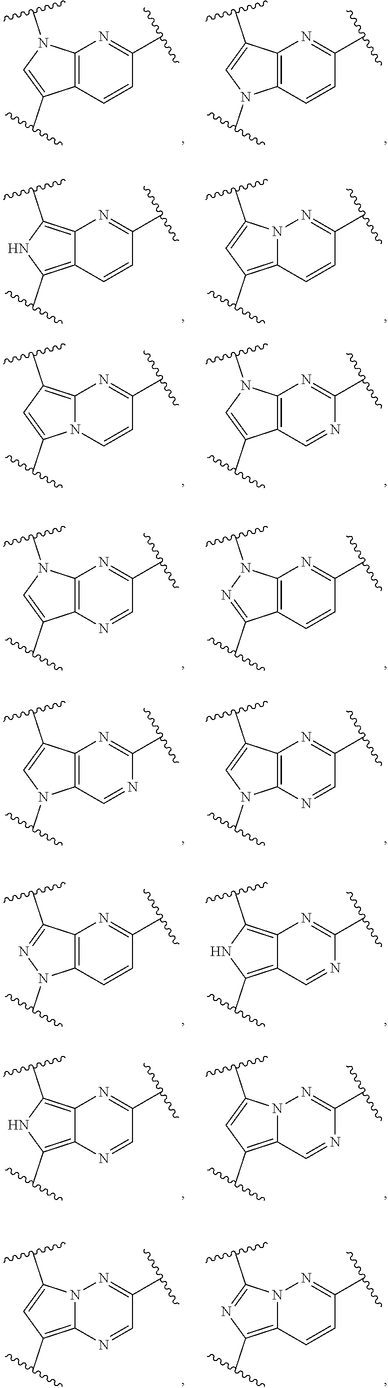 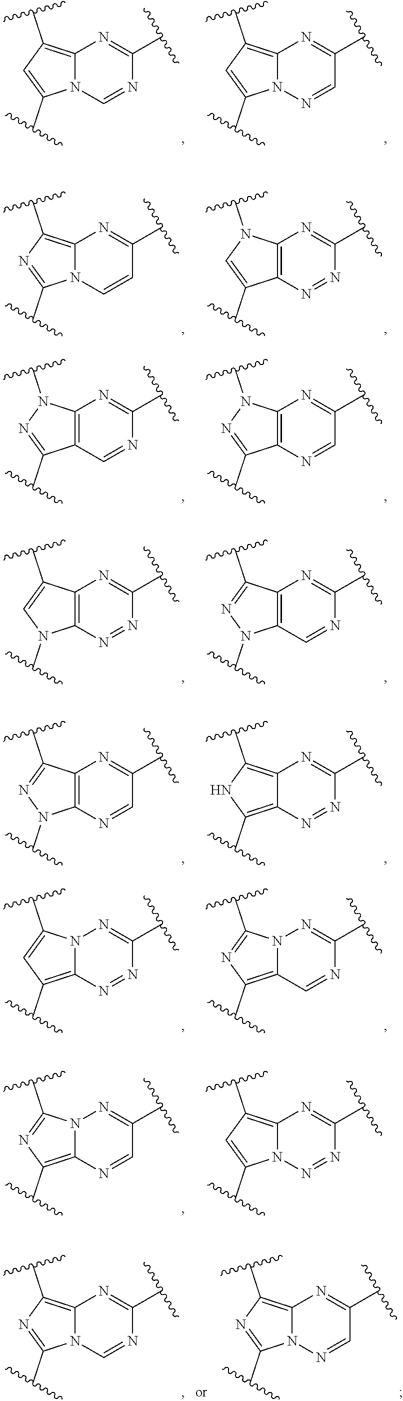
wherein each of the above-depicted groups is optionally substituted with one or more groups R^{X}.

4. The compound of claim 1 or 2, wherein the bicyclic ring system 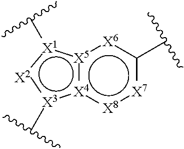
is selected from any one of the following groups: 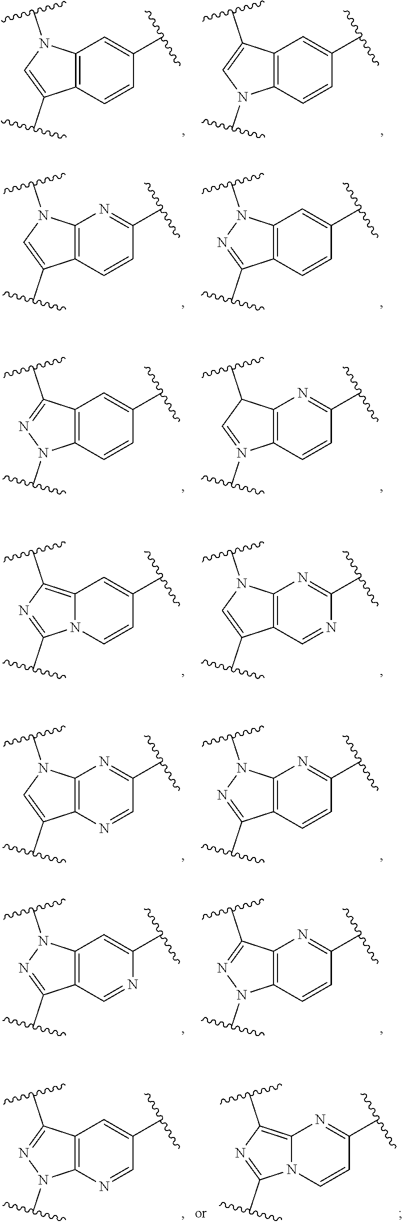
wherein each of the above-depicted groups is optionally substituted with one or more groups R^{X}.

5. The compound of any one of claims 1 to 4, wherein the bicyclic ring system 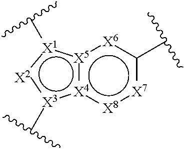
is: 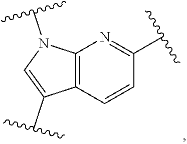 [SMILES: *c1ccc2c(*)cn(*)c2n1]
wherein the above-depicted group is optionally substituted with one or more groups R^{X}.

6. The compound of any one of claims 1 to 5, wherein R^{X1} is selected from C₁₋₅ alkyl, —(C₀₋₅ alkylene)-cycloalkyl, —(C₀₋₅ alkylene)-aryl, —(C₀₋₅ alkylene)-heterocycloalkyl, and —(C₀₋₅ alkylene)-heteroaryl, wherein said alkyl or the alkylene group in any of said —(C₀₋₅ alkylene)-cycloalkyl, said —(C₀₋₅ alkylene)-aryl, said —(C₀₋₅ alkylene)-heterocycloalkyl, or said —(C₀₋₅ alkylene)-heteroaryl is optionally substituted with one or more groups independently selected from —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), and —CN, and further wherein the cycloalkyl group in said —(C₀₋₅ alkylene)-cycloalkyl, the aryl group in said —(C₀₋₅ alkylene)-aryl, the heterocycloalkyl group in said —(C₀₋₅ alkylene)-heterocycloalkyl, and the heteroaryl group in said —(C₀₋₅ alkylene)-heteroaryl are each optionally substituted with one or more groups independently selected from C₁₋₅ alkyl, C₂₋₅ alkenyl, C₂₋₅ alkynyl, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), and —CN.

7. The compound of any one of claims 1 to 6, wherein R^{X3} is -L^{X3}-aryl, -L^{X3}-cycloalkyl or -L^{X3}-heteroaryl, wherein the aryl in said -L^{X3}-aryl, the cycloalkyl in said -L^{X3}-cycloalkyl or the heteroaryl in said -L^{X3}-heteroaryl is optionally substituted with one or more groups R^{X31}; preferably wherein R^{X3} is -L^{X3}-phenyl, wherein the phenyl in said -L^{X3}-phenyl is optionally substituted with one or more groups R^{X31}.

8. The compound of any one of claims 1 to 7, wherein R^{X3} is phenyl which is optionally substituted with one or more groups R^{X31}.

9. The compound of any one of claims 1 to 8, wherein each R^{X31} is independently selected from C₁₋₅ alkyl, halogen, C₁₋₅ haloalkyl, —O—(C₁₋₅ haloalkyl), and —CN.

10. The compound of any one of claims 1 to 9, wherein L is —CO—.

11. The compound of any one of claims 1 to 10, wherein ring A is a 5 to 14 membered heterocycloalkyl which is attached via a ring nitrogen atom to group L, wherein said heterocycloalkyl is optionally substituted with one or more groups R^{A}.

12. The compound of any one of claims 1 to 11, wherein ring A is a 5 to 14 membered heterocycloalkyl which is attached via a ring nitrogen atom to group L, wherein said heterocycloalkyl is substituted with two C₁₋₅ alkyl groups which are attached to the same ring carbon atom or is substituted with two substituents R^{A} which are attached to the same ring carbon atom and are mutually joined to form, together with the ring carbon atom that they are attached to, a C₃₋₇ cycloalkyl group, and wherein said heterocycloalkyl is optionally further substituted with one or more groups R^{A}.

13. The compound of claim 1, wherein said compound is selected from:
4-(1-(tert-butyl)-3-(4-chloro-3-fluorophenyl)-1H-pyrrolo[2,3-b]pyridine-6-carbonyl)-3,3-dimethylpiperazin-2-one;
4-(1-(tert-butyl)-3-(4-chloro-3-fluorophenyl)-1H-pyrrolo[2,3-b]pyridine-6-carbonyl)-1,3,3-trimethylpiperazin-2-one;
methyl 2-(4-(1-(tert-butyl)-3-(4-chloro-3-fluorophenyl)-1H-pyrrolo[2,3-b]pyridine-6-carbonyl)-3,3-dimethyl-2-oxopiperazin-1-yl)acetate;
2-(4-(1-(tert-butyl)-3-(4-chloro-3-fluorophenyl)-1H-pyrrolo[2,3-b]pyridine-6-carbonyl)-3,3-dimethyl-2-oxopiperazin-1-yl)acetic acid;
2-(4-(1-(tert-butyl)-3-(4-chloro-3-fluorophenyl)-1H-pyrrolo[2,3-b]pyridine-6-carbonyl)-3,3-dimethyl-2-oxopiperazin-1-yl)acetamide;
2-(4-(1-(tert-butyl)-3-(4-chloro-3-fluorophenyl)-1H-pyrrolo[2,3-b]pyridine-6-carbonyl)-3,3-dimethyl-2-oxopiperazin-1-yl)-N-methylacetamide;
2-(4-(1-(tert-butyl)-3-(4-chloro-3-fluorophenyl)-1H-pyrrolo[2,3-b]pyridine-6-carbonyl)-3,3-dimethyl-2-oxopiperazin-1-yl)-N,N-dimethylacetamide;
4-(1-(tert-butyl)-3-(4-chloro-3-fluorophenyl)-1H-pyrrolo[2,3-b]pyridine-6-carbonyl)-3,3-dimethyl-1-(2-morpholino-2-oxoethyl)piperazin-2-one;
(1-(tert-butyl)-3-(4-chloro-3-fluorophenyl)-1H-pyrrolo[2,3-b]pyridin-6-yl)((1R,3r,5S)-3-hydroxy-8-azabicyclo[3.2.1]octan-8-yl)methanone;
(1-(tert-butyl)-3-(4-chloro-3-fluorophenyl)-1H-pyrrolo[2,3-b]pyridin-6-yl)((1R,3s,5S)-3-hydroxy-8-azabicyclo[3.2.1]octan-8-yl)methanone;
8-(1-(tert-butyl)-3-(4-chloro-3-fluorophenyl)-1H-pyrrolo[2,3-b]pyridine-6-carbonyl)-2,8-diazaspiro[4.5]decan-1-one;
8-(1-(tert-butyl)-3-(4-chloro-3-fluorophenyl)-1H-pyrrolo[2,3-b]pyridine-6-carbonyl)-2,8-diazaspiro[4.5]decan-3-one;
(3aS,6aS)-1-(1-(tert-butyl)-3-(4-chloro-3-fluorophenyl)-1H-pyrrolo[2,3-b]pyridine-6-carbonyl)hexahydropyrrolo[3,4-b]pyrrol-6(1H)-one;
(1-(tert-butyl)-3-(4-chloro-3-fluorophenyl)-1H-pyrrolo[2,3-b]pyridin-6-yl)(2,2-dimethylpyrrolidin-1-yl)methanone;
4-(1-(tert-butyl)-3-(4-chloro-3-fluorophenyl)-1H-pyrrolo[2,3-b]pyridine-6-carbonyl)-1,4-diazepan-2-one;
1-(1-(tertbutyl)-3-(4-chloro-3-fluorophenyl)-1H-pyrrolo[2,3-b]pyridine-6-carbonyl)-1,4-diazepan-5-one;
((1R,5S)-3-oxa-8-azabicyclo[3.2.1]octan-8-yl)(1-(tertbutyl)-3-(4-chloro-3-fluorophenyl)-1H-pyrrolo[2,3-b]pyridin-6-yl)methanone;
4-(1-(tertbutyl)-3-(4-chloro-3-fluorophenyl)-1H-pyrrolo[2,3-b]pyridine-6-carbonyl)-1-methylpiperazin-2-one;
methyl 1-(1-(tert-butyl)-3-(4-chloro-3-fluorophenyl)-1H-pyrrolo[2,3-b]pyridine-6-carbonyl)-3,3-dimethylpiperidine-4-carboxylate;
1-(1-(tert-butyl)-3-(4-chloro-3-fluorophenyl)-1H-pyrrolo[2,3-b]pyridine-6-carbonyl)-3,3-dimethylpiperidine-4-carboxylic acid;
1-(1-(tert-butyl)-3-(4-chloro-3-fluorophenyl)-1H-pyrrolo[2,3-b]pyridine-6-carbonyl)-3,3-dimethylpiperidine-4-carboxamide;
8-(1-(tert-butyl)-3-(4-chloro-3-fluorophenyl)-1H-pyrrolo[2,3-b]pyridine-6-carbonyl)-1,3,8-triazaspiro[4.5]decane-2,4-dione;
methyl 6-(4-(1-(tert-butyl)-3-(4-chloro-3-fluorophenyl)-1H-pyrrolo[2,3-b]pyridine-6-carbonyl)-3,3-dimethylpiperazin-1-yl)-2,4-dimethylnicotinate;
6-(4-(1-(tert-butyl)-3-(4-chloro-3-fluorophenyl)-1H-pyrrolo[2,3-b]pyridine-6-carbonyl)-3,3-dimethylpiperazin-1-yl)-2,4-dimethylnicotinic acid;
4-(3-(4-chloro-3-fluorophenyl)-1-(4-methoxybenzyl)-1H-pyrrolo[2,3-b]pyridine-6-carbonyl)-3,3-dimethylpiperazin-2-one;
4-(3-(4-chloro-3-fluorophenyl)-1-(3-methoxycyclobutyl)-1H-pyrrolo[2,3-b]pyridine-6-carbonyl)-3,3-dimethylpiperazin-2-one;
4-(3-(4-chloro-3-fluorophenyl)-1-isobutyl-1H-pyrrolo[2,3-b]pyridine-6-carbonyl)-3,3-dimethylpiperazin-2-one;
4-(3-(4-chloro-3-fluorophenyl)-1-(tetrahydro-2H-pyran-4-yl)-1H-pyrrolo[2,3-b]pyridine-6-carbonyl)-3,3-dimethylpiperazin-2-one;
4-(3-(4-chloro-3-fluorophenyl)-1-(2,2,2-trifluoroethyl)-1H-pyrrolo[2,3-b]pyridine-6-carbonyl)-3,3-dimethylpiperazin-2-one;
(3-(4-chloro-3-fluorophenyl)-1-(2,2,2-trifluoroethyl)-1H-pyrrolo[2,3-b]pyridin-6-yl)(1,4-oxazepan-4-yl)methanone;
(8-oxa-3-azabicyclo[3.2.1]octan-3-yl)(3-(4-chloro-3-fluorophenyl)-1-(2,2,2-trifluoroethyl)-1H-pyrrolo[2,3-b]pyridin-6-yl)methanone;
4-(3-(4-chloro-3-fluorophenyl)-1-methyl-1H-pyrrolo[2,3-b]pyridine-6-carbonyl)-3,3-dimethylpiperazin-2-one;
4-(3-(4-chloro-3-fluorophenyl)-1-(cyclopropylmethyl)-1H-pyrrolo[2,3-b]pyridine-6-carbonyl)-3,3-dimethylpiperazin-2-one;
4-(3-(4-chloro-3-fluorophenyl)-1-isobutyl-2-methyl-1H-pyrrolo[2,3-b]pyridine-6-carbonyl)-3,3-dimethylpiperazin-2-one;
4-(3-(4-chloro-3-fluorophenyl)-1-(2-(2,2,2-trifluoroethoxy)ethyl)-1H-pyrrolo[2,3-b]pyridine-6-carbonyl)-3,3-dimethylpiperazin-2-one;
4-(1-(2-(1H-1,2,4-triazol-1-yl)ethyl)-3-(4-chloro-3-fluorophenyl)-1H-pyrrolo[2,3-b]pyridine-6-carbonyl)-3,3-dimethylpiperazin-2-one;
4-(3-(4-chloro-3-fluorophenyl)-1-(2-(2-oxopyrrolidin-1-yl)ethyl)-1H-pyrrolo[2,3-b]pyridine-6-carbonyl)-3,3-dimethylpiperazin-2-one;
4-(3-(4-chloro-3-fluorophenyl)-1-(2-ethoxyethyl)-1H-pyrrolo[2,3-b]pyridine-6-carbonyl)-3,3-dimethylpiperazin-2-one;
4-(1-isobutyl-3-(6-(trifluoromethyl)pyridin-3-yl)-1H-pyrrolo[2,3-b]pyridine-6-carbonyl)-3,3-dimethylpiperazin-2-one;
4-(1-isobutyl-3-(1-methyl-1H-pyrazol-4-yl)-1H-pyrrolo[2,3-b]pyridine-6-carbonyl)-3,3-dimethylpiperazin-2-one;
4-(3-(3,6-dihydro-2H-pyran-4-yl)-1-isobutyl-1H-pyrrolo[2,3-b]pyridine-6-carbonyl)-3,3-dimethylpiperazin-2-one;
4-(1-isobutyl-3-(tetrahydro-2H-pyran-4-yl)-1H-pyrrolo[2,3-b]pyridine-6-carbonyl)-3,3-dimethylpiperazin-2-one;
4-(3-(1,2,3,6-tetrahydropyridin-4-yl)-1-isobutyl-1H-pyrrolo[2,3-b]pyridine-6-carbonyl)-3,3-dimethylpiperazin-2-one;
4-(3-cyclohexyl-1-isobutyl-1H-pyrrolo[2,3-b]pyridine-6-carbonyl)-3,3-dimethylpiperazin-2-one;
4-(1-isobutyl-3-phenyl-pyrrolo[2,3-b]pyridine-6-carbonyl)-3,3-dimethyl-piperazin-2-one;
4-[3-(3-chlorophenyl)-1-isobutyl-pyrrolo[2,3-b]pyridine-6-carbonyl]-3,3-dimethyl-piperazin-2-one;
4-[1-isobutyl-3-(p-tolyl)pyrrolo[2,3-b]pyridine-6-carbonyl]-3,3-dimethyl-piperazin-2-one;
4-[1-isobutyl-3-(o-tolyl)pyrrolo[2,3-b]pyridine-6-carbonyl]-3,3-dimethyl-piperazin-2-one;
4-[3-(2-chlorophenyl)-1-isobutyl-pyrrolo[2,3-b]pyridine-6-carbonyl]-3,3-dimethyl-piperazin-2-one;
4-[3-(4-chlorophenyl)-1-isobutyl-pyrrolo[2,3-b]pyridine-6-carbonyl]-3,3-dimethyl-piperazin-2-one;
4-[1-isobutyl-3-[4-(trifluoromethyl)phenyl]pyrrolo[2,3-b]pyridine-6-carbonyl]-3,3-dimethyl-piperazin-2-one;
4-[1-isobutyl-3-[4-fluorophenyl]pyrrolo[2,3-b]pyridine-6-carbonyl]-3,3-dimethyl-piperazin-2-one;
4-[1-isobutyl-3-[3-(trifluoromethoxy)phenyl]pyrrolo[2,3-b]pyridine-6-carbonyl]-3,3-dimethyl-piperazin-2-one;
6-(4-(3-(tert-butyl)-1-(4-chloro-3-fluorophenyl)-1H-pyrazolo[4,3-b]pyridine-5-carbonyl)-3,3-dimethylpiperazin-1-yl)-2,4-dimethylnicotinic acid;
6-(4-(3-(tert-butyl)-1-(4-chloro-3-methoxyphenyl)-1H-pyrazolo[4,3-b]pyridine-5-carbonyl)-3,3-dimethylpiperazin-1-yl)-2,4-dimethylnicotinic acid;
4-(1-(2-(1H-pyrazol-1-yl)ethyl)-3-(4-chloro-3-fluorophenyl)-1H-pyrrolo[2,3-b]pyridine-6-carbonyl)-3,3-dimethylpiperazin-2-one;
4-(1-(4-chloro-3-fluorophenyl)-3-isobutyl-1H-pyrazolo[4,3-b]pyridine-5-carbonyl)-3,3-dimethylpiperazin-2-one;
methyl 6-(4-(1-(4-chloro-3-fluorophenyl)-3-isobutyl-1H-pyrazolo[4,3-b]pyridine-5-carbonyl)-3,3-dimethylpiperazin-1-yl)-2,4-dimethylnicotinate;
6-(4-(1-(4-chloro-3-fluorophenyl)-3-isobutyl-1H-pyrazolo[4,3-b]pyridine-5-carbonyl)-3,3-dimethylpiperazin-1-yl)-2,4-dimethylnicotinic acid;
4-(3-isobutyl-1-(6-(trifluoromethyl)pyridin-3-yl)-1H-pyrazolo[4,3-b]pyridine-5-carbonyl)-3,3-dimethylpiperazin-2-one;
4-(1-(3-chlorophenyl)-3-isobutyl-1H-pyrazolo[4,3-b]pyridine-5-carbonyl)-3,3-dimethylpiperazin-2-one;
methyl 1-(1-(4-chloro-3-fluorophenyl)-3-isobutyl-1H-pyrazolo[4,3-b]pyridine-5-carbonyl)-3,3-dimethylpiperidine-4-carboxylate;
4-(1-(4-chloro-3-fluorophenyl)-3-methyl-1H-pyrazolo[4,3-b]pyridine-5-carbonyl)-3,3-dimethylpiperazin-2-one;
4-(1-(4-chloro-3-fluorophenyl)-3-(tetrahydro-2H-pyran-4-yl)-1H-pyrazolo[4,3-b]pyridine-5-carbonyl)-3,3-dimethylpiperazin-2-one;
4-(1-(2,4-dichlorophenyl)-3-isobutyl-1H-pyrazolo[4,3-b]pyridine-5-carbonyl)-3,3-dimethylpiperazin-2-one;
4-(1-(2-chlorophenyl)-3-isobutyl-1H-pyrazolo[4,3-b]pyridine-5-carbonyl)-3,3-dimethylpiperazin-2-one;
3-(tert-butyl)-1-(4-chloro-3-fluorophenyl)-1H-pyrazolo[4,3-b]pyridine-5-carbonyl)-3,3-dimethylpiperazin-2-one;
4-(1-(4-chloro-3-fluorophenyl)-3-cyclopentyl-1H-pyrazolo[4,3-b]pyridine-5-carbonyl)-3,3-dimethylpiperazin-2-one;
4-(1-(4-chloro-3-fluorophenyl)-3-cyclopropyl-1H-pyrazolo[4,3-b]pyridine-5-carbonyl)-3,3-dimethylpiperazin-2-one;
4-(1-(4-chloro-3-fluorobenzyl)-3-isobutyl-1H-pyrazolo[4,3-b]pyridine-5-carbonyl)-3,3-dimethylpiperazin-2-one;
4-(3-isobutyl-1-(4-(trifluoromethoxy)phenyl)-1H-pyrazolo[4,3-b]pyridine-5-carbonyl)-3,3-dimethylpiperazin-2-one;
4-(3-(tert-butyl)-1-(4-chloro-3-fluorophenyl)-1H-pyrazolo[3,4-b]pyridine-5-carbonyl)-3,3-dimethylpiperazin-2-one;
1-(4-chloro-3-fluorophenyl)-3-isobutyl-6-methyl-1H-pyrazolo[4,3-b]pyridine-5-carbonyl)-3,3-dimethylpiperazin-2-one;
4-(1-isobutyl-3-(piperidin-1-yl)-1H-pyrrolo[2,3-b]pyridine-6-carbonyl)-3,3-dimethylpiperazin-2-one;
4-(3-(4-chloro-3-fluorophenyl)-1-isobutyl-1H-indazole-6-carbonyl)-3,3-dimethylpiperazin-2-one;
4-(3-(2,4-dichlorophenyl)-1-isobutyl-1H-pyrrolo[2,3-b]pyridine-6-carbonyl)-3,3-dimethylpiperazin-2-one;
4-(1-(4-chlorophenyl)-3-isobutyl-1H-pyrazolo[4,3-b]pyridine-5-carbonyl)-3,3-dimethylpiperazin-2-one;
methyl 6-(4-(1-(tert-butyl)-3-(4-chloro-3-fluorophenyl)-1H-pyrazolo[3,4-b]pyridine-6-carbonyl)-3,3-dimethylpiperazin-1-yl)-2,4-dimethylnicotinate;
6-(4-(1-(tert-butyl)-3-(4-chloro-3-fluorophenyl)-1H-pyrazolo[3,4-b]pyridine-6-carbonyl)-3,3-dimethylpiperazin-1-yl)-2,4-dimethylnicotinic acid;
4-(1-(4-chloro-3-fluorophenyl)-3-isobutyl-1H-pyrazolo[4,3-b]pyridine-5-carbonyl)piperazin-2-one;
(3-(4-chloro-3-fluorophenyl)-1-(2,2,2-trifluoroethyl)-1H-pyrazolo[4,3-c]pyridin-6-yl)(1,4-oxazepan-4-yl)methanone;
(8-oxa-3-azabicyclo[3.2.1]octan-3-yl)(3-(4-chloro-3-fluorophenyl)-1-(2,2,2-trifluoroethyl)-1H-pyrazolo[4,3-c]pyridin-6-yl)methanone;
4-(3-isobutyl-1-(6-methoxypyridin-3-yl)-1H-pyrazolo[4,3-b]pyridine-5-carbonyl)-3,3-dimethylpiperazin-2-one;
4-(3-isobutyl-1-(4-methoxyphenyl)-1H-pyrazolo[4,3-b]pyridine-5-carbonyl)-3,3-dimethylpiperazin-2-one;
8-(1-(4-chloro-3-fluorophenyl)-3-isobutyl-1H-pyrazolo[4,3-b]pyridine-5-carbonyl)-1-phenyl-1,3,8-triazaspiro[4.5]decan-4-one;
6-(4-(3-(4-chloro-3-fluorophenyl)-1-isobutyl-1H-pyrrolo[2,3-b]pyridine-6-carbonyl)-3,3-dimethylpiperazin-1-yl)-2,4-dimethylnicotinic acid;
4-(3-(4-chloro-3-fluorophenyl)-1-isopropyl-1H-pyrrolo[2,3-b]pyridine-6-carbonyl)-3,3-dimethylpiperazin-2-one;
6-(4-(3-(4-chloro-3-fluorophenyl)-1-isopropyl-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-(tetrahydro-2H-pyran-4-yl)-1H-pyrrolo[2,3-b]pyridine-6-carbonyl)-3,3-dimethylpiperazin-1-yl)-2,4-dimethylnicotinic acid;
6-(4-(1-(tert-butyl)-3-(4-chloro-3-fluorophenyl)-1H-pyrrolo[2,3-b]pyridine-6-carbonyl)piperazin-1-yl)-2,4-dimethylnicotinic acid;
6-((1-(1-(tert-butyl)-3-(4-chloro-3-fluorophenyl)-1H-pyrrolo[2,3-b]pyridine-6-carbonyl)piperidin-4-yl)amino)-2,4-dimethylnicotinic acid;
6-(6-(1-(tert-butyl)-3-(4-chloro-3-fluorophenyl)-1H-pyrrolo[2,3-b]pyridine-6-carbonyl)-3,6-diazabicyclo[3.2.0]heptan-3-yl)-2,4-dimethylnicotinic acid;
6-(4-(3-(4-chloro-3-fluorophenyl)-1-(1-hydroxy-2-methylpropan-2-yl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-(pyrazin-2-ylmethyl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-isobutyl-2-methyl-1H-pyrrolo[2,3-b]pyridine-6-carbonyl)-3,3-dimethylpiperazin-1-yl)-2,4-dimethylnicotinic acid;
4-(1-isobutyl-3-(6-methoxypyridin-3-yl)-1H-pyrrolo[2,3-b]pyridine-6-carbonyl)-3,3-dimethylpiperazin-2-one;
4-(6-(2,2-dimethyl-3-oxopiperazine-1-carbonyl)-1-isobutyl-1H-pyrrolo[2,3-b]pyridin-3-yl)benzonitrile;
4-(3-(3,4-dichlorophenyl)-1-isobutyl-1H-pyrrolo[2,3-b]pyridine-6-carbonyl)-3,3-dimethylpiperazin-2-one;
4-(3-(3,5-difluorophenyl)-1-isobutyl-1H-pyrrolo[2,3-b]pyridine-6-carbonyl)-3,3-dimethylpiperazin-2-one;
4-(3-(6-(dimethylamino)pyridin-3-yl)-1-isobutyl-1H-pyrrolo[2,3-b]pyridine-6-carbonyl)-3,3-dimethylpiperazin-2-one;
5-(6-(2,2-dimethyl-3-oxopiperazine-1-carbonyl)-1-isobutyl-1H-pyrrolo[2,3-b]pyridin-3-yl)picolinonitrile;
4-(1-isobutyl-3-(4-methoxyphenyl)-1H-pyrrolo[2,3-b]pyridine-6-carbonyl)-3,3-dimethylpiperazin-2-one;
4-(1-isobutyl-3-(2-(trifluoromethoxy)phenyl)-1H-pyrrolo[2,3-b]pyridine-6-carbonyl)-3,3-dimethylpiperazin-2-one;
4-(3-(2-fluorophenyl)-1-isobutyl-1H-pyrrolo[2,3-b]pyridine-6-carbonyl)-3,3-dimethylpiperazin-2-one;
4-(3-(3-fluorophenyl)-1-isobutyl-1H-pyrrolo[2,3-b]pyridine-6-carbonyl)-3,3-dimethylpiperazin-2-one;
4-(3-(4-chloro-2-fluorophenyl)-1-isobutyl-1H-pyrrolo[2,3-b]pyridine-6-carbonyl)-3,3-dimethylpiperazin-2-one;
4-(1-isobutyl-3-(4-(trifluoromethoxy)phenyl)-1H-pyrrolo[2,3-b]pyridine-6-carbonyl)-3,3-dimethylpiperazin-2-one;
3-(6-(2,2-dimethyl-3-oxopiperazine-1-carbonyl)-1-isobutyl-1H-pyrrolo[2,3-b]pyridin-3-yl)benzonitrile;
4-(1-isobutyl-3-(m-tolyl)-1H-pyrrolo[2,3-b]pyridine-6-carbonyl)-3,3-dimethylpiperazin-2-one;
6-(4-(3-(4-chloro-3-fluorophenyl)-1,2-dimethyl-1H-pyrrolo[2,3-b]pyridine-6-carbonyl)-3,3-dimethylpiperazin-1-yl)-2,4-dimethylnicotinic acid;
4-(3-(4-chloro-3-fluorophenyl)-1-(pyridin-4-ylmethyl)-1H-pyrrolo[2,3-b]pyridine-6-carbonyl)-3,3-dimethylpiperazin-2-one;
6-(4-(3-(4-chloro-3-fluorophenyl)-1-(1-methylcyclobutyl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-methyl-1H-pyrrolo[2,3-b]pyridine-6-carbonyl)-3,3-dimethylpiperazin-1-yl)-2,4-dimethylnicotinic acid;
6-(1-(1-(tert-butyl)-3-(4-chloro-3-fluorophenyl)-1H-pyrrolo[2,3-b]pyridine-6-carbonyl)piperidine-4-carboxamido)-2,4-dimethylnicotinic acid;
2-((1R,5S,6s)-3-(1-(tert-butyl)-3-(4-chloro-3-fluorophenyl)-1H-pyrrolo[2,3-b]pyridine-6-carbonyl)-3-azabicyclo[3.1.0]hexan-6-yl)acetic acid;
2-(1-(1-(tert-butyl)-3-(4-chloro-3-fluorophenyl)-1H-pyrrolo[2,3-b]pyridine-6-carbonyl)piperidin-4-yl)acetic acid
6-(4-(3-(4-chloro-3-fluorophenyl)-1-(1-ethoxy-2-methylpropan-2-yl)-1H-pyrrolo[2,3-b]pyridine-6-carbonyl)-3,3-dimethylpiperazin-1-yl)-2,4-dimethylnicotinic acid;
6-(4-(1-(tert-butyl)-3-(4-chloro-3-fluorophenyl)-1H-pyrrolo[2,3-b]pyridine-6-carbonyl)-1,4-diazepan-1-yl)-2,4-dimethylnicotinic acid;
6-(4-(3-(4-chloro-3-fluorophenyl)-1-ethyl-1H-pyrrolo[2,3-b]pyridine-6-carbonyl)-3,3-dimethylpiperazin-1-yl)-2,4-dimethylnicotinic acid;
(R)-6-(4-(1-(tert-butyl)-3-(4-chloro-3-fluorophenyl)-1H-pyrrolo[2,3-b]pyridine-6-carbonyl)-3-methylpiperazin-1-yl)-2,4-dimethylnicotinic acid;
6-(4-(3-(4-chloro-3-fluorophenyl)-1-(2-(pyridin-2-yl)propan-2-yl)-1H-pyrrolo[2,3-b]pyridine-6-carbonyl)-3,3-dimethylpiperazin-1-yl)-2,4-dimethylnicotinic acid;
6-(4-(1-(tert-butyl)-3-(4-chloro-3-fluorophenyl)-1H-pyrrolo[2,3-b]pyridine-6-carbonyl)-2-(methoxymethyl)piperazin-1-yl)-2,4-dimethylnicotinic acid;
6-(4-(1-(carboxymethyl)-3-(4-chloro-3-fluorophenyl)-1H-pyrrolo[2,3-b]pyridine-6-carbonyl)-3,3-dimethylpiperazin-1-yl)-2,4-dimethylnicotinic acid;
6-(4-(3-(3-chloro-4-fluorophenyl)-1-(2-methoxyethyl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-(pyridin-3-ylmethyl)-1H-pyrrolo[2,3-b]pyridine-6-carbonyl)-3,3-dimethylpiperazin-1-yl)-2,4-dimethylnicotinic acid;
(S)-6-(4-(3-(4-chloro-3-fluorophenyl)-1-(1-methoxypropan-2-yl)-1H-pyrrolo[2,3-b]pyridine-6-carbonyl)-3,3-dimethylpiperazin-1-yl)-2,4-dimethylnicotinic acid;
6-(4-(1-(tert-butyl)-3-(4-chloro-3-fluorophenyl)-1H-pyrrolo[2,3-b]pyridine-6-carbonyl)-3,3-dimethylpiperazin-1-yl)nicotinic acid;
6-(4-(1-(tert-butyl)-3-(4-chloro-3-fluorophenyl)-1H-pyrrolo[2,3-b]pyridine-6-carbonyl)-3,3-dimethylpiperazin-1-yl)nicotinamide;
6-(4-(1-(tert-butyl)-3-(4-chloro-3-fluorophenyl)-1H-pyrrolo[2,3-b]pyridine-6-carbonyl)-3,3-dimethylpiperazin-1-yl)-N,N-dimethylnicotinamide;
(S)-6-(4-(3-(4-chloro-3-fluorophenyl)-1-(1-methoxypropan-2-yl)-1H-pyrrolo[2,3-b]pyridine-6-carbonyl)-3,3-dimethylpiperazin-1-yl)-2,4-dimethylnicotinic acid;
6-(4-(3-(3,4-difluorophenyl)-1-isobutyl-1H-pyrrolo[2,3-b]pyridine-6-carbonyl)-3,3-dimethylpiperazin-1-yl)-2,4-dimethylnicotinic acid;
6-(4-(3-(3-fluoro-4-methylphenyl)-1-isobutyl-1H-pyrrolo[2,3-b]pyridine-6-carbonyl)-3,3-dimethylpiperazin-1-yl)-2,4-dimethylnicotinic acid;
6-(4-(1-isobutyl-3-(3,4,5-trifluorophenyl)-1H-pyrrolo[2,3-b]pyridine-6-carbonyl)-3,3-dimethylpiperazin-1-yl)-2,4-dimethylnicotinic acid;
6-(4-(3-(3-fluoro-4-(trifluoromethyl)phenyl)-1-isobutyl-1H-pyrrolo[2,3-b]pyridine-6-carbonyl)-3,3-dimethylpiperazin-1-yl)-2,4-dimethylnicotinic acid;
4-(3-(3,4-dimethoxyphenyl)-1-isobutyl-1H-pyrrolo[2,3-b]pyridine-6-carbonyl)-3,3-dimethylpiperazin-2-one;
6-(4-(1-(tert-butyl)-3-(4-chloro-3-fluorophenyl)-1H-pyrrolo[2,3-b]pyridine-6-carbonyl)-2-(methoxymethyl)piperazin-1-yl)-2,4-dimethylnicotinic acid;
6-((1-(1-(tert-butyl)-3-(4-chloro-3-fluorophenyl)-1H-pyrrolo[2,3-b]pyridine-6-carbonyl)piperidin-4-yl)(methyl)amino)-2,4-dimethylnicotinic acid;
6-((1S,4S)-5-(1-(tert-butyl)-3-(4-chloro-3-fluorophenyl)-1H-pyrrolo[2,3-b]pyridine-6-carbonyl)-2,5-diazabicyclo[2.2.1]heptan-2-yl)-2,4-dimethylnicotinic acid;
6-(4-(3-(4-chloro-3-fluorophenyl)-1-(pyridin-4-ylmethyl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-(pyridin-2-ylmethyl)-1H-pyrrolo[2,3-b]pyridine-6-carbonyl)-3,3-dimethylpiperazin-1-yl)-2,4-dimethylnicotinic acid;
4-(3-(4-chloro-3-fluorophenyl)-1-(1-methoxy-2-methylpropan-2-yl)-1H-pyrrolo[2,3-b]pyridine-6-carbonyl)-3,3-dimethylpiperazin-2-one;
6-(4-(3-(4-chloro-3-fluorophenyl)-1-(1-methoxy-2-methylpropan-2-yl)-1H-pyrrolo[2,3-b]pyridine-6-carbonyl)-3,3-dimethylpiperazin-1-yl)-2,4-dimethylnicotinic acid;
(R)-6-((1-(1-(tert-butyl)-3-(4-chloro-3-fluorophenyl)-1H-pyrrolo[2,3-b]pyridine-6-carbonyl)pyrrolidin-3-yl)(methyl)amino)-2,4-dimethylnicotinic acid;
(S)-6-((1-(1-(tert-butyl)-3-(4-chloro-3-fluorophenyl)-1H-pyrrolo[2,3-b]pyridine-6-carbonyl)pyrrolidin-3-yl)(methyl)amino)-2,4-dimethylnicotinic acid;
6-(4-(1-(tert-butyl)-3-(4-chloro-3-fluorophenyl)-1H-pyrrolo[2,3-b]pyridine-6-carbonyl)-2-(hydroxymethyl)piperazin-1-yl)-2,4-dimethylnicotinic acid;
6-(4-(3-(4-chloro-3-fluorophenyl)-1-(pyrimidin-2-ylmethyl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-((2,5-dimethyloxazol-4-yl)methyl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-(cyclopropylmethyl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-((6-methoxypyridin-2-yl)methyl)-1H-pyrrolo[2,3-b]pyridine-6-carbonyl)-3,3-dimethylpiperazin-1-yl)-2,4-dimethylnicotinic acid;
1-(1-(tert-butyl)-3-(4-chloro-3-fluorophenyl)-1H-pyrrolo[2,3-b]pyridine-6-carbonyl)piperidine-4-carboxylic acid;
2-(1-(1-(tert-butyl)-3-(4-chloro-3-fluorophenyl)-1H-pyrrolo[2,3-b]pyridine-6-carbonyl)piperidin-4-yl)acetamide;
2-(1-(1-(tert-butyl)-3-(4-chloro-3-fluorophenyl)-1H-pyrrolo[2,3-b]pyridine-6-carbonyl)piperidin-4-yl)-N,N-dimethylacetamide;
(R)-6-(4-(1-(sec-butyl)-3-(4-chloro-3-fluorophenyl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-(1,3-dimethoxypropan-2-yl)-1H-pyrrolo[2,3-b]pyridine-6-carbonyl)-3,3-dimethylpiperazin-1-yl)-2,4-dimethylnicotinic acid;
1′-(1-(tert-butyl)-3-(4-chloro-3-fluorophenyl)-1H-pyrrolo[2,3-b]pyridine-6-carbonyl)-4,6-dimethyl-1′,2′,3′,6′-tetrahydro-[2,4′-bipyridine]-5-carboxylic acid;
6-(1-(1-(tert-butyl)-3-(4-chloro-3-fluorophenyl)-1H-pyrrolo[2,3-b]pyridine-6-carbonyl)piperidin-4-yl)-2,4-dimethylnicotinic acid;
6-(4-(1-(tert-butyl)-3-(4-chloro-3-fluorophenyl)-1H-pyrrolo[2,3-b]pyridine-6-carbonyl)-3,3-dimethylpiperazine-1-carbonyl)nicotinic acid;
6-(4-(1-isobutyl-3-(6-(trifluoromethyl)pyridin-3-yl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-(cyclobutylmethyl)-1H-pyrrolo[2,3-b]pyridine-6-carbonyl)-3,3-dimethylpiperazin-1-yl)-2,4-dimethylnicotinic acid;
(S)-6-(4-(1-(sec-butyl)-3-(4-chloro-3-fluorophenyl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-(cyclobutylmethyl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-isobutyl-5-methyl-1H-pyrrolo[2,3-b]pyridine-6-carbonyl)-3,3-dimethylpiperazin-1-yl)-2,4-dimethylnicotinic acid;
6-(4-(5-(4-chloro-3-fluorophenyl)-7-isobutyl-7H-pyrrolo[2,3-d]pyrimidine-2-carbonyl)-3,3-dimethyl piperazin-1-yl)-2,4-dimethylnicotinic acid;
2-(2-(4-(1-(tert-butyl)-3-(4-chloro-3-fluorophenyl)-1H-pyrrolo[2,3-b]pyridine-6-carbonyl)-3,3-dimethylpiperazin-1-yl)pyridin-4-yl)-2-methylpropanenitrile;
2-(2-(4-(1-(tert-butyl)-3-(4-chloro-3-fluorophenyl)-1H-pyrrolo[2,3-b]pyridine-6-carbonyl)-3,3-dimethylpiperazin-1-yl)pyridin-4-yl)-2-methylpropanamide;
diethyl (6-(4-(1-(tert-butyl)-3-(4-chloro-3-fluorophenyl)-1H-pyrrolo[2,3-b]pyridine-6-carbonyl)-3,3-dimethylpiperazin-1-yl)pyridin-3-yl)phosphonate;
(S)-6-(4-(1-(tert-butyl)-3-(4-chloro-3-fluorophenyl)-1H-pyrrolo[2,3-b]pyridine-6-carbonyl)-3-methylpiperazin-1-yl)-2,4-dimethylnicotinic acid;
6-(4-(3-(4-chloro-3-fluorophenyl)-1-(2-hydroxy-2-methylpropyl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-((3-methyloxetan-3-yl)methyl)-1H-pyrrolo[2,3-b]pyridine-6-carbonyl)-3,3-dimethylpiperazin-1-yl)-2,4-dimethylnicotinic acid;
(S)-6-(4-(3-(4-chloro-3-fluorophenyl)-1-((tetrahydrofuran-3-yl)methyl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-((4-methyltetrahydro-2H-pyran-4-yl)methyl)-1H-pyrrolo[2,3-b]pyridine-6-carbonyl)-3,3-dimethylpiperazin-1-yl)-2,4-dimethylnicotinic acid;
(R)-6-(4-(3-(4-chloro-3-fluorophenyl)-1-((tetrahydrofuran-3-yl)methyl)-1H-pyrrolo[2,3-b]pyridine-6-carbonyl)-3,3-dimethylpiperazin-1-yl)-2,4-dimethylnicotinic acid;
6-(4-(3-(tert-butyl)-1-(4-chloro-3-fluorophenyl)-1H-pyrrolo[3,2-b]pyridine-5-carbonyl)-3,3-dimethylpiperazin-1-yl)-2,4-dimethylnicotinic acid;
1-((1-(1-(tert-butyl)-3-(4-chloro-3-fluorophenyl)-1H-pyrrolo[2,3-b]pyridine-6-carbonyl)piperidin-4-yl)methyl)-1H-pyrazole-4-carboxylic acid;
6-(4-(3-(4-chloro-3-fluorophenyl)-1-isobutyl-4-methyl-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-isobutyl-4-methyl-1H-pyrrolo[2,3-b]pyridine-6-carbonyl)piperazin-1-yl)-2,4-dimethylnicotinic acid;
6-(4-(7-(4-chloro-3-fluorophenyl)-5-isobutyl-5H-pyrrolo[2,3-b]pyrazine-3-carbonyl)-3,3-dimethylpiperazin-1-yl)-2,4-dimethylnicotinic acid;
6-(4-(1-(tert-butyl)-3-(3,4-difluorophenyl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-((6-methylpyridin-2-yl)methyl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-((3-methylpyridin-2-yl)methyl)-1H-pyrrolo[2,3-b]pyridine-6-carbonyl)-3,3-dimethylpiperazin-1-yl)-2,4-dimethylnicotinic acid;
(6-(4-(1-(tert-butyl)-3-(4-chloro-3-fluorophenyl)-1H-pyrrolo[2,3-b]pyridine-6-carbonyl)-3,3-dimethylpiperazin-1-yl)pyridin-3-yl)phosphonic acid;
ethyl hydrogen (6-(4-(1-(tert-butyl)-3-(4-chloro-3-fluorophenyl)-1H-pyrrolo[2,3-b]pyridine-6-carbonyl)-3,3-dimethylpiperazin-1-yl)pyridin-3-yl)phosphonate;
8-(1-(tert-butyl)-3-(4-chloro-3-fluorophenyl)-1H-pyrrolo[2,3-b]pyridine-6-carbonyl)-3-methyl-1,3,8-triazaspiro[4.5]decane-2,4-dione;
8-(1-(tert-butyl)-3-(4-chloro-3-fluorophenyl)-1H-pyrrolo[2,3-b]pyridine-6-carbonyl)-1,3-dimethyl-1,3,8-triazaspiro[4.5]decane-2,4-dione;
6-((1-(1-(tert-butyl)-3-(4-chloro-3-fluorophenyl)-1H-pyrrolo[2,3-b]pyridine-6-carbonyl)-3-methylpiperidin-4-yl)amino)-2,4-dimethylnicotinic acid;
6-((1-(1-(tert-butyl)-3-(4-chloro-3-fluorophenyl)-1H-pyrrolo[2,3-b]pyridine-6-carbonyl)-3-methylpiperidin-4-yl)(methyl)amino)-2,4-dimethylnicotinic acid;
8-(1-(tert-butyl)-3-(4-chloro-3-fluorophenyl)-1H-pyrrolo[2,3-b]pyridine-6-carbonyl)-6,6-dimethyl-1,3,8-triazaspiro[4.5]decane-2,4-dione;
8-(1-(tert-butyl)-3-(4-chloro-3-fluorophenyl)-1H-pyrrolo[2,3-b]pyridine-6-carbonyl)-6,6-difluoro-1,3,8-triazaspiro[4.5]decane-2,4-dione;
8-(1-(tert-butyl)-3-(4-chloro-3-fluorophenyl)-1H-pyrrolo[2,3-b]pyridine-6-carbonyl)-1,8-diazaspiro[4.5]decan-2-one;
methyl 2-(1-(3-(4-chloro-3-fluorophenyl)-1-isobutyl-1H-indole-6-carbonyl)piperidin-4-yl)acetate;
2-(1-(3-(4-chloro-3-fluorophenyl)-1-isobutyl-1H-indole-6-carbonyl)piperidin-4-yl)acetic acid;
6-(4-(3-(4-chloro-3-fluorophenyl)-1-isobutyl-2-(trifluoromethyl)-1H-pyrrolo[2,3-b]pyridine-6-carbonyl)-3,3-dimethylpiperazin-1-yl)-2,4-dimethylnicotinic acid;
2-(1-(3-acetyl-1-(4-chloro-3-fluorophenyl)-1H-indole-5-carbonyl)piperidin-4-yl)acetic acid;
8-(1-(tert-butyl)-3-(4-chloro-3-fluorophenyl)-1H-pyrrolo[2,3-b]pyridine-6-carbonyl)-1-isopropyl-1,3,8-triazaspiro[4.5]decan-4-one;
5-(4-(1-(tert-butyl)-3-(4-chloro-3-fluorophenyl)-1H-pyrrolo[2,3-b]pyridine-6-carbonyl)piperazin-1-yl)pyridazin-3(2H)-one;
8-(1-(tert-butyl)-3-(4-chloro-3-fluorophenyl)-1H-pyrrolo[2,3-b]pyridine-6-carbonyl)-1-methyl-1,3,8-triazaspiro[4.5]decan-4-one;
4-(4-(1-(tert-butyl)-3-(4-chloro-3-fluorophenyl)-1H-pyrrolo[2,3-b]pyridine-6-carbonyl)-3,3-dimethylpiperazin-1-yl)-2,6-dimethylbenzoic acid;
(S)-6-(4-(3-(4-chloro-3-fluorophenyl)-1-((tetrahydrofuran-2-yl)methyl)-1H-pyrrolo[2,3-b]pyridine-6-carbonyl)-3,3-dimethylpiperazin-1-yl)-2,4-dimethylnicotinic acid;
(R)-6-(4-(3-(4-chloro-3-fluorophenyl)-1-((tetrahydrofuran-2-yl)methyl)-1H-pyrrolo[2,3-b]pyridine-6-carbonyl)-3,3-dimethylpiperazin-1-yl)-2,4-dimethylnicotinic acid;
6-(4-(3-(4-chloro-3-fluorophenyl)-1-((5-methylpyridin-2-yl)methyl)-1H-pyrrolo[2,3-b]pyridine-6-carbonyl)-3,3-dimethylpiperazin-1-yl)-2,4-dimethylnicotinic acid;
(1-(tert-butyl)-3-(4-chloro-3-fluorophenyl)-1H-pyrrolo[2,3-b]pyridin-6-yl)(1,1-dioxidothiomorpholino)methanone;
8-(4-(1-(tert-butyl)-3-(4-chloro-3-fluorophenyl)-1H-pyrrolo[2,3-b]pyridine-6-carbonyl)-3,3-dimethylpiperazin-1-yl)-8-oxooctanoic acid;
ethyl 6-(4-(8-(tert-butyl)-6-(4-chlorophenyl)imidazo[1,5-a]pyrimidine-2-carbonyl)-3,3-dimethylpiperazin-1-yl)nicotinate;
6-(4-(8-(tert-butyl)-6-(4-chlorophenyl)imidazo[1,5-a]pyrimidine-2-carbonyl)-3,3-dimethylpiperazin-1-yl)nicotinic acid;
ethyl 6-(4-(3-(4-chlorophenyl)-1-isobutylimidazo[1,5-a]pyridine-7-carbonyl)-3,3-dimethylpiperazin-1-yl)nicotinate;
6-(4-(3-(4-chlorophenyl)-1-isobutylimidazo[1,5-a]pyridine-7-carbonyl)-3,3-dimethylpiperazin-1-yl)nicotinic acid;
6-(4-(1-(tert-butyl)-3-(3-fluoro-4-(trifluoromethyl)phenyl)-1H-pyrrolo[2,3-b]pyridine-6-carbonyl)-3,3-dimethylpiperazin-1-yl)-2,4-dimethylnicotinic acid;
4-(3-(tert-butyl)-1-(4-chloro-3-fluorophenyl)-1H-pyrrolo[3,2-b]pyridine-5-carbonyl)-3,3-dimethylpiperazin-2-one;
2-(1-(3-(tert-butyl)-1-(4-chloro-3-fluorophenyl)-1H-pyrrolo[3,2-b]pyridine-5-carbonyl)piperidin-4-yl)acetic acid;
4-(3-(benzo[d][1,3]dioxol-5-yl)-1-isobutyl-1H-pyrrolo[2,3-b]pyridine-6-carbonyl)-3,3-dimethylpiperazin-2-one;
4-(3-(benzofuran-2-yl)-1-isobutyl-1H-pyrrolo[2,3-b]pyridine-6-carbonyl)-3,3-dimethylpiperazin-2-one;
4-(3-(5-fluoro-1H-indol-2-yl)-1-isobutyl-1H-pyrrolo[2,3-b]pyridine-6-carbonyl)-3,3-dimethylpiperazin-2-one;
4-(1-isobutyl-3-(naphthalen-2-yl)-1H-pyrrolo[2,3-b]pyridine-6-carbonyl)-3,3-dimethylpiperazin-2-one;
4-(1-(4-chloro-3-fluorophenyl)-3-isobutyl-1H-indazole-5-carbonyl)-3,3-dimethylpiperazin-2-one;
or a pharmaceutically acceptable salt or solvate of any of the aforementioned compounds.

14. A compound as defined in any one of claims 1 to 13, wherein said compound is conjugated via a linker to a membrane anchor.

15. A pharmaceutical composition comprising the compound of any one of claims 1 to 14 and a pharmaceutically acceptable excipient.

16. The compound of any one of claims 1 to 14 or the pharmaceutical composition of claim 15 for use in the treatment or prevention of pain, an autoimmune disorder, an autoinflammatory disorder, an inflammatory disorder, a central nervous system disorder, spinal cord injury, a metabolic disorder, a gastrointestinal disorder, a cardiovascular disorder, a fibrotic disorder, a respiratory disorder, a skin disorder, an allergic disorder, or cancer.

17. The compound of any one of claims 1 to 14 or the pharmaceutical composition of claim 15 for use in the treatment or prevention of neuropathic pain, inflammatory pain, cancer pain, post-operative incision pain, fracture pain, osteoporotic fracture pain, gout joint pain, chronic pain, spinal cord injury, atopic dermatitis, contact dermatitis, dry skin dermatitis, seborrhoeic dermatitis, arthritis, rheumatoid arthritis, osteoarthritis, psoriasis, psoriatic arthritis, multiple sclerosis, non-alcoholic steatohepatitis, obesity, diabetes, adipose inflammation, pancreatitis, metabolic syndrome, PAR-2 associated metabolic dysfunction, periodontitis, gingivitis, inflammatory bowel disease, Crohn's disease, ulcerative colitis, peptic ulcer disease, infectious enteritis, irritable bowel syndrome, atherosclerosis, asthma, interstitial lung disease, pulmonary fibrosis, rheumatoid arthritis-associated interstitial lung disease, liver fibrosis, cystic fibrosis, renal fibrosis, peritoneal fibrosis, pancreatic fibrosis, intestinal fibrosis, cardiac fibrosis, skin fibrosis, systemic lupus erythematosus, scleroderma, skin eczema, acne, rosacea, post-inflammatory hyperpigmentation, lichen planus, pruritus, polymyositis, vasculitis, Wegener's granulomatosis, Netherton syndrome, dermatomyositis, uveitis, liver cirrhosis, Alzheimer's disease, Parkinson's disease, dust mite allergy, cockroach allergy, or allergic asthma.

18. The compound of any one of claims 1 to 14 or the pharmaceutical composition of claim 15 for use in the treatment or prevention of cancer.

19. The compound for use according to claim 18 or the pharmaceutical composition for use according to claim 18, wherein said cancer is selected from colorectal cancer, colon cancer, gastric cancer, rectal cancer, liver cancer, breast cancer, pancreatic cancer, cervical cancer, prostate cancer, ovarian cancer, endometrial cancer, uterine sarcoma, germ cell cancer, esophageal cancer, leukemia, lung cancer, adrenal gland cancer, bile duct cancer, bladder cancer, head and neck cancer, kidney cancer, lymphoma, mesothelioma, sarcoma, melanoma, thyroid carcinoma, thymus cancer, and glioblastoma.

20. The compound for use according to claim 18 or 19 or the pharmaceutical composition for use according to claim 18 or 19, wherein said compound or said pharmaceutical composition is to be administered in combination with one or more immune checkpoint inhibitors.

21. The compound for use according to claim 20 or the pharmaceutical composition for use according to claim 20, wherein said immune checkpoint inhibitors are selected from anti-CTLA-4 antibodies, anti-PD-1 antibodies, anti-PD-L1 antibodies, anti-TIGIT antibodies, and anti-LAG3 antibodies.

22. The compound for use according to claim 20 or 21 or the pharmaceutical composition for use according to claim 20 or 21, wherein said immune checkpoint inhibitors are selected from ipilimumab, tremelimumab, nivolumab, pembrolizumab, pidilizumab, cemiplimab, dostarlimab, spartalizumab, camrelizumab, sintilimab, tislelizumab, toripalimab, zimberelimab, AMP-224, AMP-514, JTX-4014, INCMGA00012, APE02058, atezolizumab, avelumab, durvalumab, KN035, CK-301, BMS-936559, MEDI4736, MPDL3280A, MDX-1105, MEDI6469, bintrafusp alfa, tiragolumab, vibostolimab, domvanalimab, etigilimab, BMS-986207, EOS-448, COM902, ASP8374, SEA-TGT, BGB-A1217, IBI-939, M6223, relatlimab, ieramilimab, encelimab, tebotelimab, REGN3767, FS118, IMP701, and IMP731.

23. In vitro use of a compound as defined in any one of claims 1 to 14 as a PAR-2 inhibitor.
